(19) United States
(12) Patent Application Publication
(10) Pub. No.: US 2025/0301900 A1
(43) Pub. Date: Sep. 25, 2025

(54) ORGANIC COMPOUND AND ELECTRONIC ELEMENT AND ELECTRONIC APPARATUS COMPRISING SAME

(71) Applicant: SHAANXI LIGHTE OPTOELECTRONICS MATERIAL CO., LTD., Xi'an, Shaanxi (CN)

(72) Inventor: Fumin YUE, Xi'an, Shaanxi (CN)

(21) Appl. No.: 18/862,227

(22) PCT Filed: Sep. 18, 2023
(86) PCT No.: PCT/CN2023/119534

§ 371 Date: Nov. 1, 2024

(30) Foreign Application Priority Data
Jan. 18, 2023 (CN) 202310074995.5

(51) Int. Cl. H10K 85/60; C07B 59/00; C07C 211/54; C07C 211/58; C07C 211/61; C07D 209/86; C07D 209/88; C07D 307/91; C07D 333/76; C07D 405/12; C07D 409/12; H10K 50/15

(52) U.S. Cl. CPC H10K 85/633; C07B 59/001; C07B 59/002; C07C 211/54; C07C 211/58; C07C 211/61; C07D 209/86; C07D 209/88; C07D 307/91; C07D 333/76; C07D 405/12; C07D 409/12; H10K 85/636; C07B 2200/05; C07C 2603/18; C07C 2603/26; C07C 2603/40; C07C 2603/94; H10K 50/156; H10K 85/615; H10K 85/626; H10K 85/6572; H10K 85/6574; H10K 85/6576

(57) ABSTRACT

The present application relates to an organic compound and an electronic element and an electronic apparatus comprising same. The structural formula of the organic compound of the present application comprises structures represented by a Formula 1 and a Formula 2, and the organic compound is applied to organic light-emitting devices, so that the performance of the devices can be significantly improved.

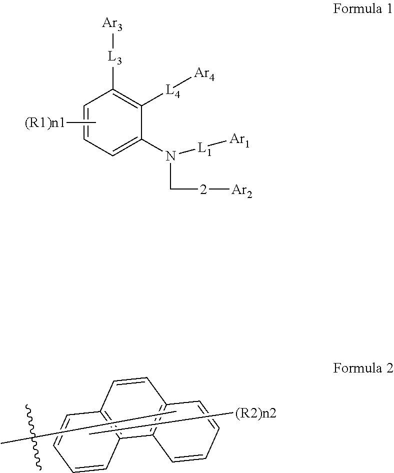

### CROSS-REFERENCE TO RELATED APPLICATIONS

The present application claims the priority of Chinese patent application No. 202310074995.5 filed on Jan. 18, 2023, which is incorporated herein by reference in its entirety as a part of the present application.

### FIELD OF THE INVENTION

The present application belongs to the technical field of organic electroluminescence, and in particular to an organic compound and an electronic element and an electronic apparatus comprising the same.

### BACKGROUND OF THE INVENTION

Organic electroluminescent devices (OLEDs) are devices fabricated by depositing organic materials between two metal electrodes through spin coating or vacuum evaporation. The classic three-layer organic electroluminescent device comprises a hole transport layer, an organic light-emitting layer, and an electron transport layer. Holes generated at the anode via the hole transport layer and electrons generated at the cathode via the electron transport layer are combined to form excitons in the organic light-emitting layer, which subsequently emit light. The light of the organic electroluminescent device can be modulated by altering the materials of the organic light-emitting layer as required. Compared with liquid crystal display technology, OLED display technology has numerous advantages such as self-luminescence, non-radiation, lightweight, thin thickness, wide viewing angle, broad color gamut, stable color rendering, rapid response speed, strong environmental adaptability, and the capability for flexible displays. Consequently, OLED display technology has been receiving increasing attention and corresponding technological investment.

Currently, it is disclosed in many existing technologies that aromatic amine compounds are utilized as hole transport materials or auxiliary hole transport layer materials in OLED devices, which can regulate the transport and injection of carriers into the organic light-emitting layer. However, it is still necessary for the continued development of novel hole transport materials to further enhance the performance of organic electroluminescent devices.

### SUMMARY OF THE INVENTION

The objective of the present application is to provide an organic compound, and an electronic element and an electronic apparatus comprising the same. Using the organic compound in organic electroluminescent devices can improve the performance of the devices.

A first aspect of the present application provides an organic compound having a structure shown in a Formula 1:

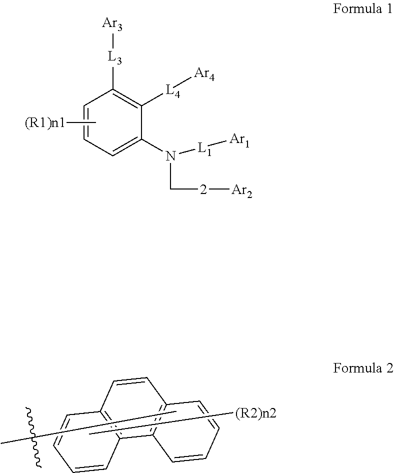

wherein, Ar₁ is selected from a substituted or unsubstituted aryl having 6 to 12 carbon atoms, or a substituted or unsubstituted heteroaryl having 6 to 18 carbon atoms;
Ar₂, Ar₃ and Ar₄ are the same or different, and are each independently selected from a substituted or unsubstituted aryl having 6 to 40 carbon atoms, or a substituted or unsubstituted heteroaryl having 3 to 40 carbon atoms, and one or two of Ar₃ and Ar₄ are selected from the group represented by a Formula 2;
L₁ is selected from a single bond, or a substituted or unsubstituted arylene having 6 to 12 carbon atoms;
L₂, L₃ and L₄ are the same or different, and are each independently selected from a single bond, a substituted or unsubstituted arylene having 6 to 30 carbon atoms, or a substituted or unsubstituted heteroarylene having 3 to 30 carbon atoms;
R₁ and R₂ are the same or different, and each independently selected from a deuterium, a halogen group, a cyano, an alkyl having 1 to 5 carbon atoms, or an aryl having 6 to 12 carbon atoms; or
any two adjacent R₁ are interconnected to form a ring, or any two adjacent R₂ are interconnected to form a ring;
n₁ is the number of R₁, n₁ is selected from 0, 1, 2, or 3; when n₁ is greater than 1, any two R₁ are the same or different;
n₂ is the number of R₂, n₂ is selected from 0, 1, 2, 3, 4, 5, 6, 7, 8, or 9; when n₂ is greater than 1, any two R₂ are the same or different;
the substituent(s) of Ar₁ and L₁ are the same or different, and are each independently selected from a deuterium, a halogen group, a cyano, an alkyl having 1 to 10 carbon atoms, or an aryl having 6 to 12 carbon atoms;
the substituent(s) of Ar₂, Ar₃, Ar₄, L₂, L₃ and L₄ are the same or different, and are each independently selected from a deuterium, a halogen group, a cyano, an alkyl having 1 to 10 carbon atoms, an aryl having 6 to 20 carbon atoms, a deuterated aryl having 6 to 20 carbon atoms, a haloaryl having 6 to 20 carbon atoms, or a heteroaryl having 3 to 20 carbon atoms;
optionally, any two adjacent substituents of Ar₁ form a saturated or unsaturated 3-membered to 15-membered ring; optionally, any two adjacent substituents of Ar₂ form a saturated or unsaturated 3-membered to 15-membered ring.

A second aspect of the present application provides an electronic element, comprising an anode and a cathode disposed opposite to each other, and a functional layer disposed between the anode and the cathode; the functional layer comprises the organic compound described in the first aspect of the present application.

A third aspect of the present application provides an electronic apparatus, comprising the electronic element described in the second aspect of the present application.

The organic compound of the present application is centered around a benzene ring, in which an aromatic amine is attached to the central benzene ring, and a phenanthrene group and another aromatic group are attached to the ortho- and meta-positions of the aromatic amine. The spatial configuration of the molecule is adjusted through the large planar conjugation characteristics of phenanthrene, elevating the glass transition temperature of the material. The indirect connection of the electron-rich phenanthryl with the arylamino through the benzene ring further effectively improves the hole mobility of the molecule and reduces the potential barrier for carriers to be injected into the organic light-emitting layer, thereby lowering the voltage of the device and enhancing the luminous efficiency of the device. Moreover, the consecutive adjacent substituents on the central benzene ring improve the spatial distortion of the molecule, which can elevate the glass transition temperature of the material, ensuring the formation of a stable amorphous thin film during vapor deposition, thereby prolonging the lifespan of the device. Additionally, in order to enhance the overall thermal stability of the molecule, the substituent attached to the aromatic amine is specifically limited to a designated aromatic group. Therefore, using the organic compound of the present application as a hole transport material can significantly enhance the luminous efficiency and the lifespan of the device, while reducing the operating voltage of the device.

The other features and advantages of the present application will be described in detail in the following Detailed Description of the Embodiments.

### BRIEF DESCRIPTION OF THE DRAWINGS

The drawings are used for a further understanding of the present application and constitute a part of the specification and are used to explain the present application together with the following specific embodiments, but do not constitute a limitation of the present application.

FIG. 1 is a schematic structural diagram of an organic electroluminescent device according to one embodiment of the present application.

FIG. 2 is a schematic diagram of a first electronic apparatus according to one embodiment of the present application.

FIG. 3 is a schematic structural diagram of a photoelectric conversion device according to one embodiment of the present application.

FIG. 4 is a schematic diagram of a second electronic apparatus according to one embodiment of the present application.

### LIST OF REFERENCE SIGNS

100: Anode 200: Cathode 300: Functional layer 310: Hole injection layer 320: Hole transport layer 321: First hole transport layer 322: Second hole transport layer 330: Organic light-emitting layer 340: Hole blocking layer 350: Electron transport layer 360: Electron injection layer 370: Photoelectric conversion layer 400: First electronic apparatus 500: Second electronic apparatus

### DETAILED DESCRIPTION OF THE EMBODIMENTS

Exemplary embodiments will now be described more comprehensively with reference to the accompanying drawings. The exemplary embodiments, however, can be implemented in a variety of forms and should not be interpreted as being limited to the examples set forth herein. On the contrary, these embodiments are provided to make the present application more comprehensive and complete, and to convey the concepts of these exemplary embodiments fully to those skill in the art. Features, structures, or characteristics described herein can be combined in one or more embodiment(s) in any suitable manner. In the following description, many specific details are provided to give a full understanding of the examples of the present application.

In a first aspect, the present application provides an organic compound having a structure shown in a Formula 1:

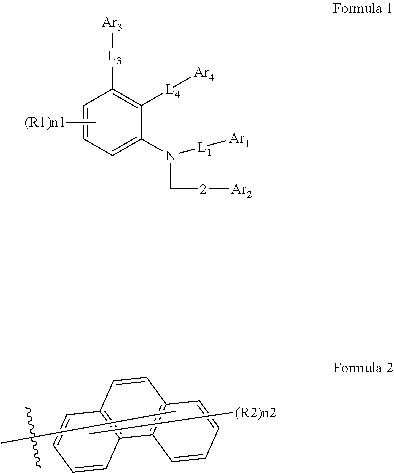

wherein, Ar₁ is selected from a substituted or unsubstituted aryl having 6 to 12 carbon atoms, or a substituted or unsubstituted heteroaryl having 6 to 18 carbon atoms;
Ar₂, Ar₃ and Ar₄ are the same or different, and are each independently selected from a substituted or unsubstituted aryl having 6 to 40 carbon atoms, or a substituted or unsubstituted heteroaryl having 3 to 40 carbon atoms, and one or two of Ar₃ and Ar₄ are selected from the group represented by a Formula 2;
L₁ is selected from a single bond, or a substituted or unsubstituted arylene having 6 to 12 carbon atoms;
L₂, L₃ and L₄ are the same or different, and are each independently selected from a single bond, a substituted or unsubstituted arylene having 6 to 30 carbon atoms, or a substituted or unsubstituted heteroarylene having 3 to 30 carbon atoms;
R₁ and R₂ are the same or different, and each independently selected from a deuterium, a halogen group, a cyano, an alkyl having 1 to 5 carbon atoms, or an aryl having 6 to 12 carbon atoms; or
any two adjacent R₁ are interconnected to form a ring, or any two adjacent R₂ are interconnected to form a ring;
n₁ is the number of R₁, n₁ is selected from 0, 1, 2, or 3; when n₁ is greater than 1, any two R₁ are the same or different;
n₂ is the number of R₂, n₂ is selected from 0, 1, 2, 3, 4, 5, 6, 7, 8, or 9; when n₂ is greater than 1, any two R₂ are the same or different;
the substituent(s) of Ar₁ and L₁ are the same or different, and are each independently selected from a deuterium, a halogen group, a cyano, an alkyl having 1 to 10 carbon atoms, or an aryl having 6 to 12 carbon atoms;
the substituent(s) of Ar₂, Ar₃, Ar₄, L₂, L₃ and L₄ are the same or different, and each independently selected from a deuterium, a halogen group, a cyano, an alkyl having 1 to 10 carbon atoms, an aryl having 6 to 20 carbon atoms, a deuterated aryl having 6 to 20 carbon atoms, a haloaryl having 6 to 20 carbon atoms, or a heteroaryl having 3 to 20 carbon atoms; optionally, any two adjacent substituents of Ar₁ form a saturated or unsaturated 3-membered to 15-membered ring;
optionally, any two adjacent substituents of Ar₂ form a saturated or unsaturated 3-membered to 15-membered ring.

In the present application, the terms “optional” and “optionally” mean that the event or circumstance described later can but do not necessarily occur, and the description includes the situations where the event or circumstance occurs or does not occur. For example, “optionally, any two adjacent substituents XX form a ring” means that these two substituents may form a ring but not necessarily, including scenarios both where two adjacent substituents form a ring and where two adjacent substituents do not form a ring. For instance, “any two adjacent substituents of Ar₂ can form a saturated or unsaturated 3-membered to 15-membered ring” means that any two adjacent substituents of Ar₂ may be interconnected to form a saturated or unsaturated 3-membered to 15-membered ring, or any two adjacent substituents of Ar₂ may exist independently of each other.

“Any two adjacent substituents” can include having two substituents on the same atom, and can also include having one substituent on each of adjacent atoms; among them, when there are two substituents on the same atom, the two substituents can form a saturated or unsaturated ring with the atom they are linked to together; when two adjacent atoms each has a substituent, these two substituents can be fused into a ring.

In the present application, fluorenyl can be substituted by one or more substituent(s). When the above-mentioned fluorenyl is substituted, the substituted fluorenyl may be:

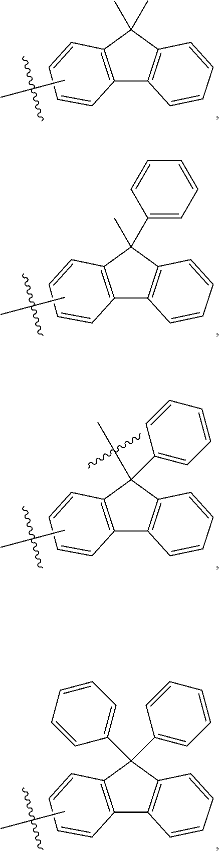

etc, but are not limited thereto.

In the present application, the descriptive expressions “ . . . each independently” and “ . . . respectively independently” and “ . . . independently selected from” can be interchanged and all these expressions should be interpreted in a broad sense. They can both refer to specific options expressed by the same symbol in different groups are mutually non-influential, and to specific options expressed by the same symbols within the same group are mutually non-influential. The groups each may be the same or different. For example,

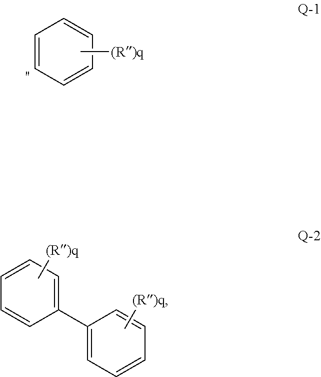

in which each q is independently 0, 1, 2, and 3, and each R″ is independently selected from a hydrogen, a deuterium, a fluorine, and a chlorine” means that Formula Q-1 represents that there are q substituents R″ on the benzene ring, and each R″ can be the same or different, with mutual non-influence between the options for each R″; Formula Q-2 represents that there are q substituents R″ on each benzene ring of biphenyl, and the number q of R″ substituents on the two benzene rings can be the same or different, with mutual non-influence between the options for each R″.

In the present application, the term “substituted or unsubstituted” means that the functional group defined by the term may or may not have a substituent (hereinafter referred to as Rc for ease of description). For example, “a substituted or unsubstituted aryl” refers to an aryl having a substituent Rc or an unsubstituted aryl. Among them, the above substituent, i.e., Rc, may be, for example, a deuterium, a halogen group, a cyano, a heteroaryl, an alkyl, a trialkylsilyl, an alkyl, a haloalkyl, and a cycloalkyl, etc.

In the present application, the number of carbon atoms of a substituted or unsubstituted functional group refers to the total number of carbon atoms. For example, if L is a substituted arylene having 12 carbon atoms, the total number of carbon atoms of the arylene and its substituents is 12.

In the present application, an aryl refers to an optional functional group or a substituent derived from an aromatic carbon ring. An aryl may be a monocyclic aryl (e.g., phenyl) or a polycyclic aryl. In other words, an aryl may be a monocyclic aryl, a fused aryl, two or more monocyclic aryls linked by carbon-carbon bond conjugation, a monocyclic aryl and a fused aryl linked by carbon-carbon bond conjugation, or two or more fused aryls linked by carbon-carbon bond conjugation. That is, unless otherwise specified, two or more aromatic groups linked by carbon-carbon bond conjugation may also be regarded as an aryl in the present application. Among them, a fused aryl may include, for example, a bicyclic fused aryl (e.g., naphthyl), a tricyclic fused aryl (e.g., phenanthryl, fluorenyl, and anthryl), etc. For example, in the present application, biphenyl, terphenyl and the like belong to an aryl. Examples of an aryl include, but are not limited to, a phenyl, a naphthyl, a fluorenyl, an anthryl, a phenanthryl, a biphenyl, a terphenyl, a benzo[9,10]phenanthryl, a spirobifluorenyl, a pyrenyl, a benzofluoranthryl, a chrysenyl, etc. In the present application, “an arylene” involved refers to a divalent group formed by further removing one hydrogen atom from an aryl.

In the present application, a substituted aryl may mean that one or more hydrogen atom(s) in the aryl are replaced by a group such as a deuterium atom, a halogen group, a cyano, an aryl, a heteroaryl, a trialkylsilyl, an alkyl, a cycloalkyl, and a haloalkyl, etc. It should be understood that the number of carbon atoms in a substituted aryl refers to the total number of carbon atoms of an aryl and the substituents on the aryl. For example, a substituted aryl having 18 carbon atoms, refers to the total number of carbon atoms of the aryl and the substituents thereof is 18.

In the present application, “a heteroaryl” refers to a monovalent aromatic ring containing at least one heteroatom or a derivative thereof. The heteroatom may be at least one of B, O, N, P, Si, Se, and S. A heteroaryl may be a monocyclic heteroaryl or a polycyclic heteroaryl. In other words, a heteroaryl may be a single aromatic ring system, or multiple aromatic ring systems linked by carbon-carbon bond conjugation, with any of the aromatic ring systems being an aromatic monocyclic ring or an aromatic fused ring. For example, a heteroaryl may include, thienyl, furyl, pyrrolyl, imidazolyl, thiazolyl, oxazolyl, oxadiazolyl, triazolyl, pyridyl, dipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, N-phenylcarbazolyl, N-pyridylcarbazolyl, N-methylcarbazolyl, etc, but are not limited thereto. Among them, the thienyl, the furyl, the phenanthrolinyl and the like are heteroaryl of a single aromatic ring system, while N-phenylcarbazolyl and N-pyridylcarbazolyl are a heteroaryl of polycyclic systems conjugately linked by carbon-carbon bond conjugation. In the present application, “a heteroarylene” involved refers to a divalent group formed by further removing one hydrogen atom from a heteroaryl.

In the present application, a substituted heteroaryl may mean that one or more hydrogen atom(s) in the heteroaryl are replaced by a group such as a deuterium atom, a halogen group, a cyano, an aryl, a heteroaryl, a trialkylsilyl, an alkyl, a cycloalkyl, or a haloalkyl. It should be understood that the number of carbon atoms of the substituted heteroaryl refers to the total number of carbon atoms of the heteroaryl and the substituents thereof.

In the present application, the number of carbon atoms of an aryl as the substituent in Ar₁, Ar₂, Ar₃, Ar₄, L₁, L₂, L₃ and L₄ may be 6 to 20, for example, the number of carbon atoms may be 6, 7, 8, 9, 10, 11, 12, 13, 14, 15, 16, 17, 18, 19, or 20, and specific examples of the aryl as the substituent include, but are not limited to a phenyl, a biphenyl, a naphthyl, a fluorenyl, a phenanthryl, an anthryl, and a chrysenyl.

In the present application, the number of carbon atoms of a heteroaryl as the substituent for Ar₁, Ar₂, Ar₃, Ar₄, L₁, L₂, L₃ and L₄ may be 12 to 20. For example, the number of carbon atoms may be 12, 13, 14, 15, 16, 17, 18, 19, or 20, and specific examples of the heteroaryl as the substituent include, but are not limited to a carbazolyl, a dibenzofuranyl, or a dibenzothienyl.

In the present application, a non-positional bond involves a single bond “A” extending from the ring system, which represents that one end of the linkage bond can link to any position in the ring system through which the bond passes, and the other end links to the rest of the compound molecule.

In the present application, the number of carbon atoms of an alkyl having 1 to 10 carbon atoms may be, for example, 1, 2, 3, 4, 5, 6, 7, 8, 9, or 10. Specific examples of an alkyl include, but are not limited to a methyl, an ethyl, a n-propyl, an isopropyl, a n-butyl, an isobutyl, a tert-butyl, a n-pentyl, an isopentyl, a neopentyl, a n-hexyl, a n-octyl, a 2-ethylhexyl, a nonyl, a decyl, or a 3,7-dimethyloctyl, etc.

In the present application, a halogen group may be for example, a fluorine, a chlorine, a bromine, or an iodine.

In the present application, specific examples of a trialkylsilyl include, but are not limited to, a trimethylsilyl or a triethylsilyl.

In the present application, specific examples of a haloalkyl include, but are not limited to, a trifluoromethyl.

For example, as shown in Formula (f) below, the naphthyl represented by Formula (f) is linked to other positions of the molecule through two non-positional bonds passing through the two rings, which indicates any of possible linkages forms shown in Formulae (f-1) to (f-10):

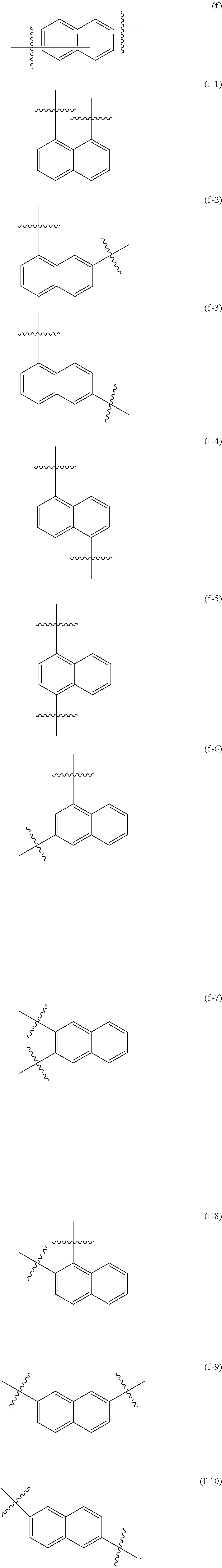

As another example, as shown in Formula (X′) below, the dibenzofuranyl group represented by Formula (X′) is linked to other positions of the molecule via a non-positional bond extending from the center of benzene ring on one side, which indicates any of possible linkages forms shown in Formulae (X′-1) to (X′-4):

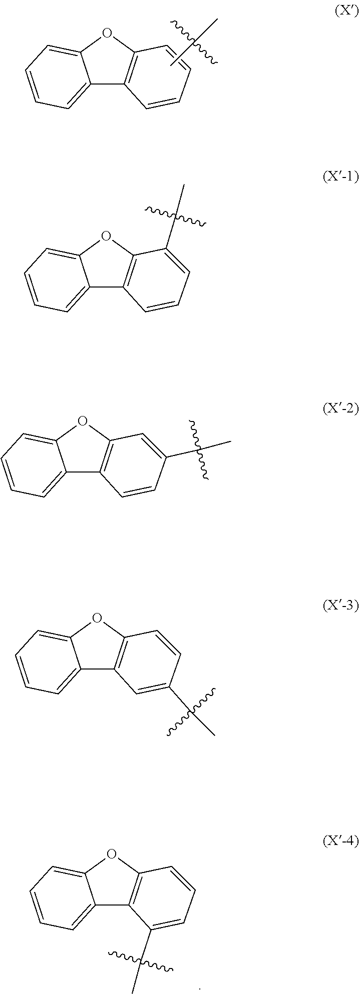

Optionally, the Formula 1 is selected from the structure represented by a Formula I-1, a Formula I-2, a Formula I-3, a Formula I-4, a Formula I-5, a Formula I-6, a Formula I-7, a Formula 1-8, or a Formula 1-9:

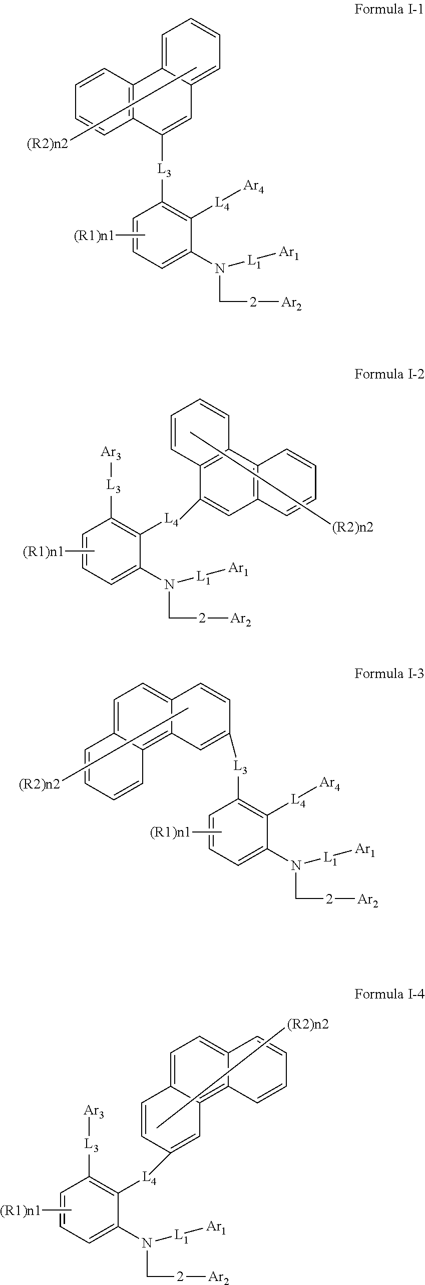

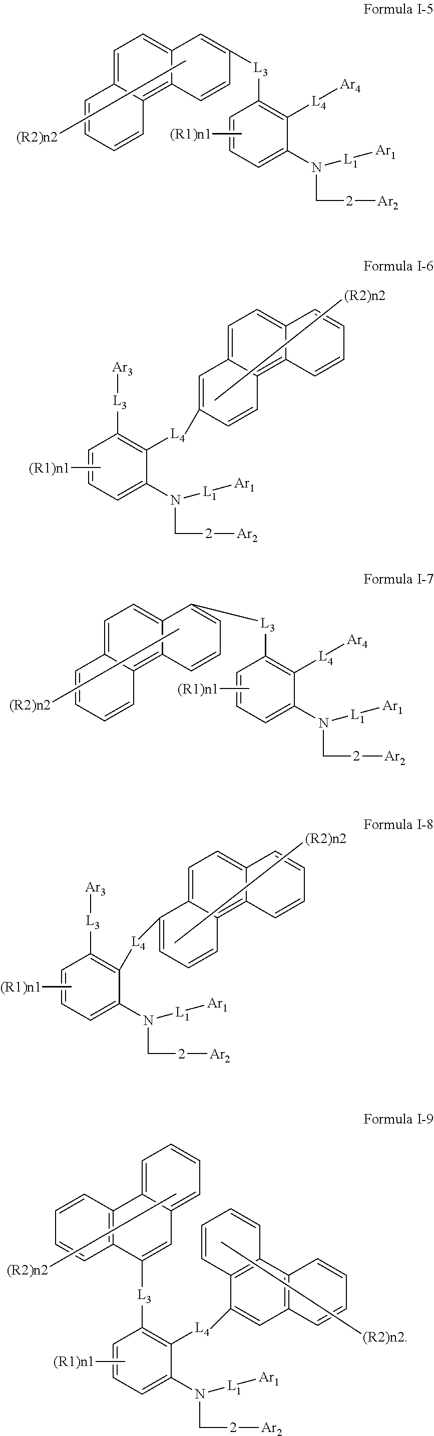

Further optionally, the Formula 1 is selected from the structure represented by a Formula I-1:

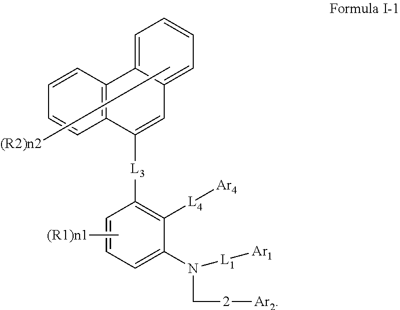

In one embodiment of the present application, Ar₁ is selected from a substituted or unsubstituted phenyl, a substituted or unsubstituted naphthyl, a substituted or unsubstituted biphenyl, a substituted or unsubstituted dibenzofuranyl, a substituted or unsubstituted dibenzothienyl, or a substituted or unsubstituted carbazolyl.

Optionally, the substituent(s) of Ar₁ are each independently selected from a deuterium, a fluorine, a cyano, a methyl, an ethyl, an isopropyl, a tert-butyl, or a phenyl.

Optionally, Ar₁ is selected from the group consisting of the following groups:

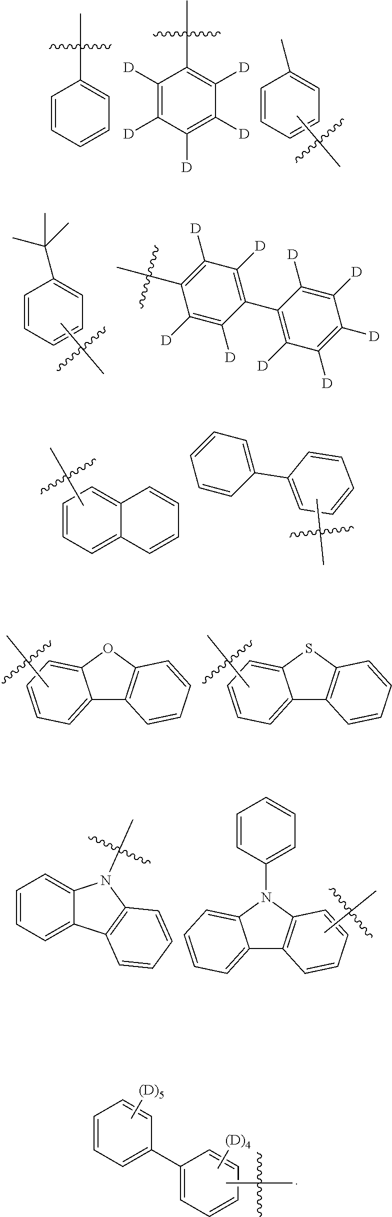

In some embodiments of the present application, Ar₁ is selected from the following groups:

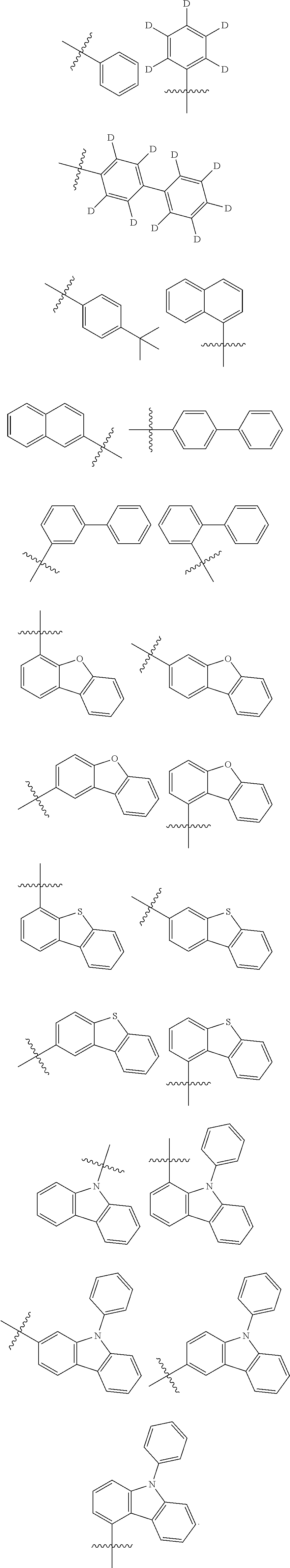

In one embodiment of the present application, Ar₂ is selected from a substituted or unsubstituted aryl having 6 to 25 carbon atoms, or a substituted or unsubstituted heteroaryl having 12 to 25 carbon atoms.

Optionally, the substituent(s) of Ar₂ are each independently selected from a deuterium, a fluorine, a cyano, an alkyl having 1 to 5 carbon atoms, a trimethylsilyl, a trifluoromethyl, an aryl having 6 to 12 carbon atoms, a deuterated aryl having 6 to 12 carbon atoms, a haloaryl having 6 to 12 carbon atoms, or a heteroaryl having 12 to 18 carbon atoms; optionally, any two adjacent substituents of Ar₂ form a fluorene ring.

In one embodiment of the present application, Ar₂ is selected from a substituted or unsubstituted phenyl, a substituted or unsubstituted naphthyl, a substituted or unsubstituted biphenyl, a substituted or unsubstituted fluorenyl, a substituted or unsubstituted benzofluorenyl, a substituted or unsubstituted phenanthryl, a substituted or unsubstituted terphenyl, a substituted or unsubstituted spirobifluorenyl, a substituted or unsubstituted dibenzofuranyl, a substituted or unsubstituted dibenzothienyl, or a substituted or unsubstituted carbazolyl.

Optionally, the substituent(s) of Ar₂ are each independently selected from a deuterium, a fluorine, a cyano, a methyl, an ethyl, an isopropyl, a tert-butyl, a trideuterated methyl, a trimethylsilyl, a trifluoromethyl, a phenyl, a naphthyl, a biphenyl, a dibenzofuranyl, or a dibenzothienyl.

Optionally, Ar₂ is selected from a substituted or unsubstituted group W, wherein the unsubstituted group W is selected from the following groups:

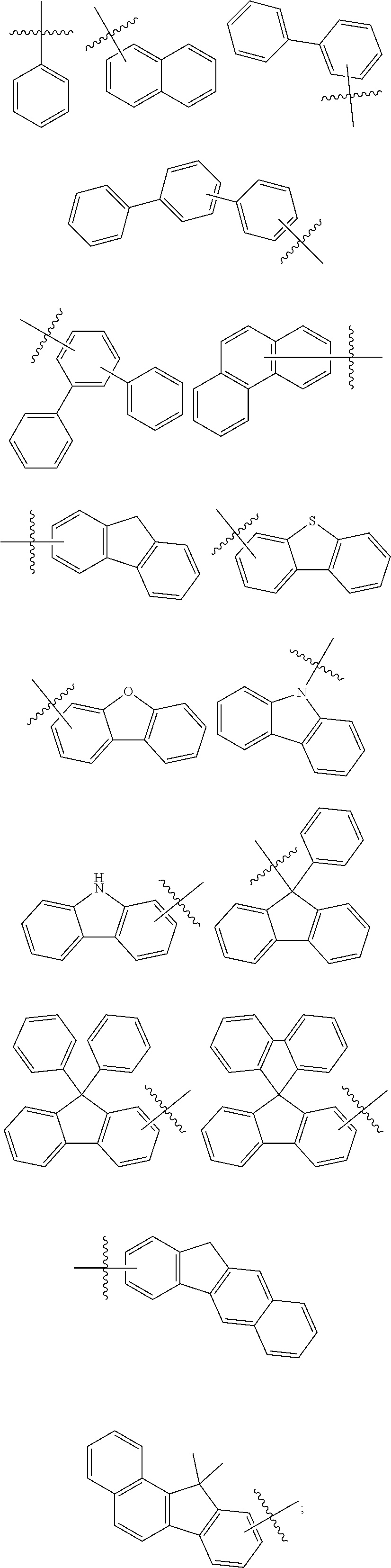

wherein, the substituted group W has one or more substituent(s), and the substituents are each independently selected from a deuterium, a fluorine, a cyano, a methyl, an ethyl, an isopropyl, a tert-butyl, a trideuterated methyl, a trimethylsilyl, a trifluoromethyl, a phenyl, a naphthyl, a biphenyl, a dibenzofuranyl, or a dibenzothienyl, and when the number of the substituents is greater than 1, the substituents are each the same or different.

Optionally, Ar₂ is selected from the group consisting of the following groups:

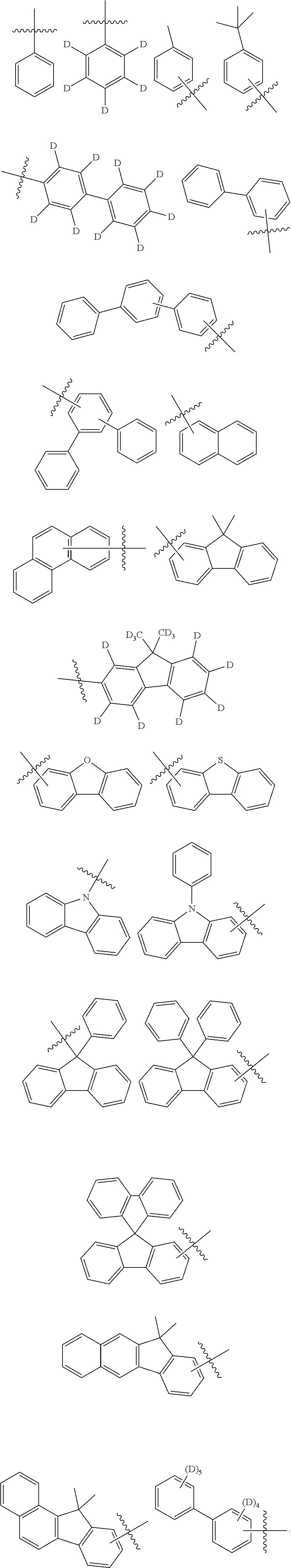

Optionally, Ar₂ is selected from the following groups:

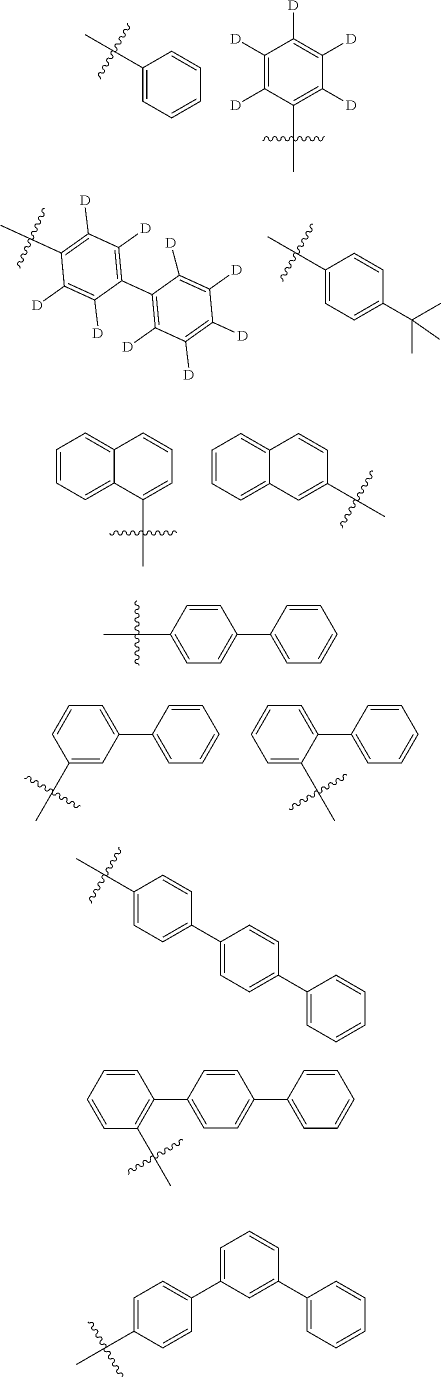

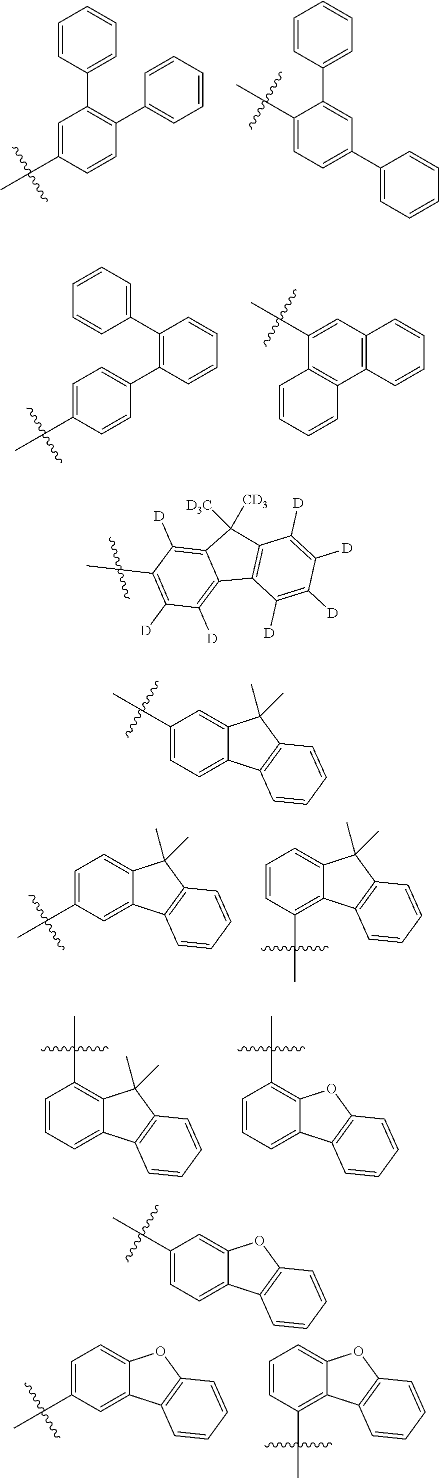

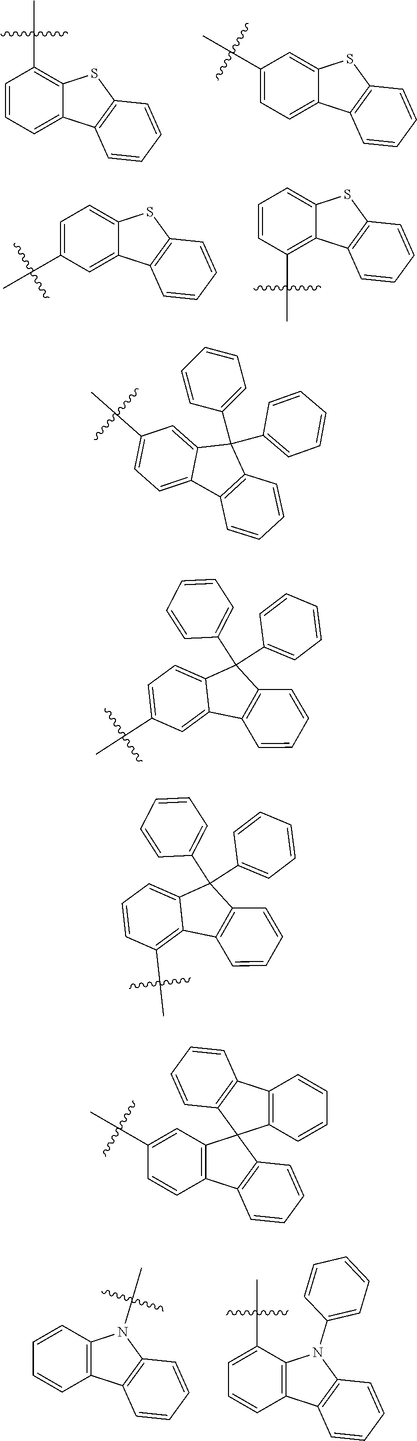

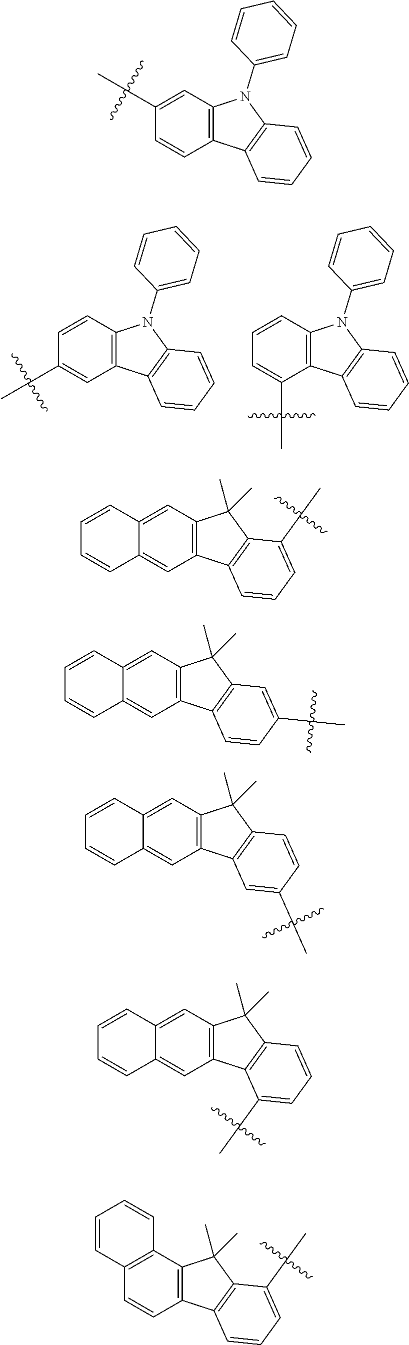

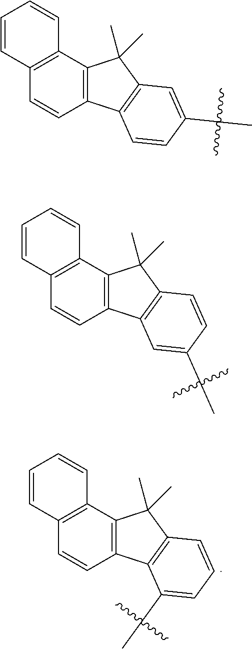 [SMILES: *c1ccc2c(c1)C(C)(C)c1c-2ccc2ccccc12 ; *c1ccc2c(c1)-c1ccc3ccccc3c1C2(C)C ; *c1cccc2c1-c1ccc3ccccc3c1C2(C)C]

In one embodiment of the present application, Ar₃ and Ar₄ are each independently selected from a substituted or unsubstituted aryl having 6 to 25 carbon atoms, or a substituted or unsubstituted heteroaryl having 12 to 25 carbon atoms. For example, Ar₃ and Ar₄ are each independently selected from a substituted or unsubstituted aryl having 6, 7, 8, 9, 10, 11, 12, 13, 14, 15, 16, 17, 18, 19, 20, 21, 22, 23, 24, or 25 carbon atoms, or a substituted or unsubstituted heteroaryl having 12, 13, 14, 15, 16, 17, 18, 19, 20, or 25, and one or two of Ar₃ and Ar₄ are selected from the group represented by the Formula 2.

In the present application, the structure represented by Formula 2 is a substituted or unsubstituted phenanthryl with 0 to 9 R₂(s), which belongs to a substituted or unsubstituted aryl.

In the organic compound of the present application, at least one of Ar₃ and Ar₄ is selected from the group represented by the Formula 2, and the substituent(s) of the group which is not selected from the Formula 2 are each independently selected from a deuterium, a fluorine, a cyano, an alkyl having 1 to 5 carbon atoms, a trimethylsilyl, a trifluoromethyl, an aryl having 6 to 12 carbon atoms, a deuterated aryl having 6 to 12 carbon atoms, a haloaryl having 6 to 12 carbon atoms, or a heteroaryl having 12 to 18 carbon atoms.

In one embodiment of the present application, Ar₃ and Ar₄ are the same or different, and are each independently selected from a substituted or unsubstituted phenyl, a substituted or unsubstituted naphthyl, a substituted or unsubstituted biphenyl, a substituted or unsubstituted fluorenyl, a substituted or unsubstituted terphenyl, a substituted or unsubstituted spirobifluorenyl, a substituted or unsubstituted dibenzofuranyl, a substituted or unsubstituted dibenzothienyl, a substituted or unsubstituted carbazolyl, or the group represented by the Formula 2, and one or two of Ar₃ and Ar₄ are selected from the group represented by the Formula 2.

Optionally, the substituent(s) of one of Ar₃ and Ar₄ which is not selected from the group represented by the Formula 2 are each independently selected from a deuterium, a fluorine, a cyano, a methyl, an ethyl, an isopropyl, a tert-butyl, a trideuterated methyl, a trimethylsilyl, a trifluoromethyl, a phenyl, a naphthyl, a biphenyl, a dibenzofuranyl, or a dibenzothienyl.

In some embodiments of the present application, Formula 2

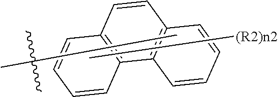

is selected from the following groups:

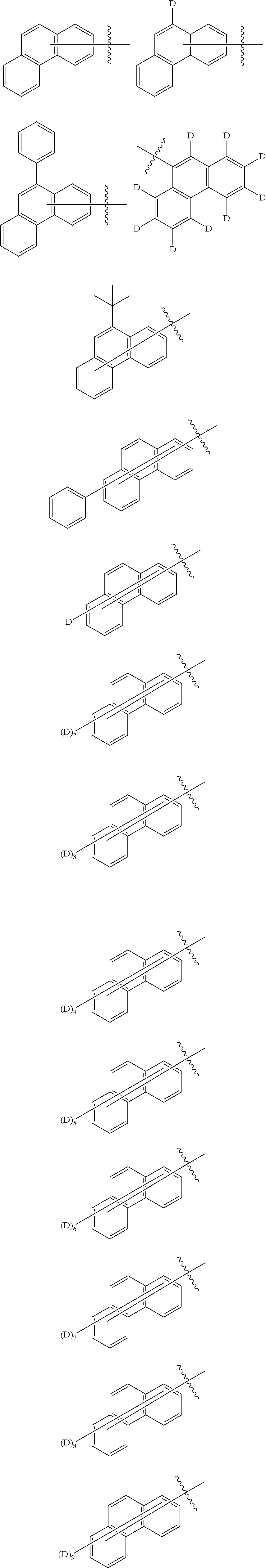

Optionally, Formula 2

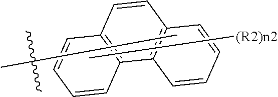

is selected from the following groups:

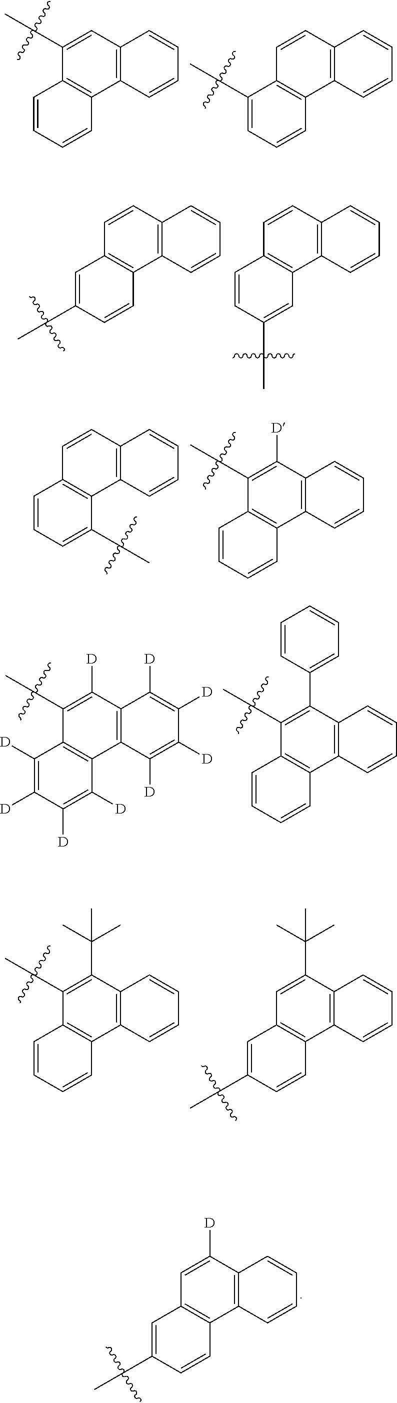

In some embodiments of the present application, Ar₄ is the group represented by Formula

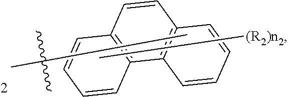

and Ar₃ is selected from the following groups:

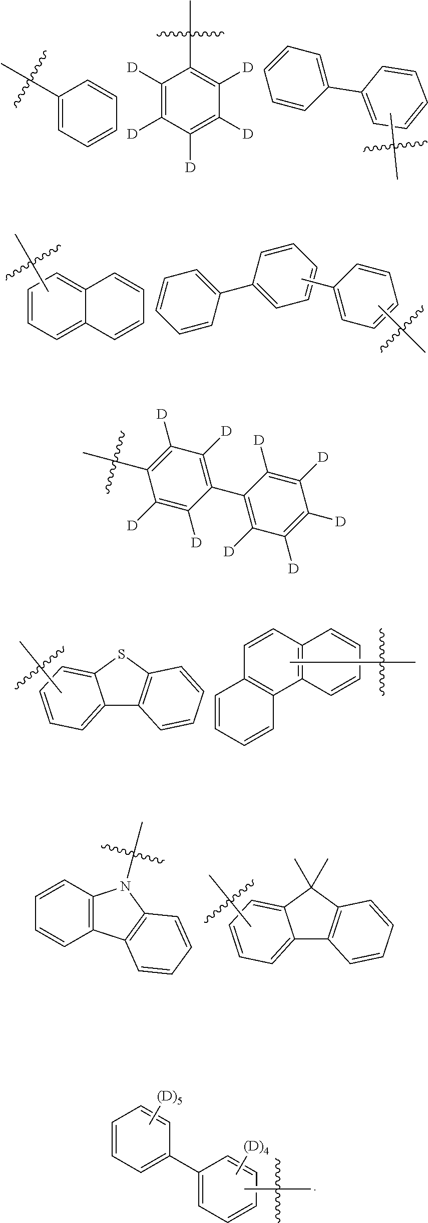

or Ar₃ is the group represented by Formula 2

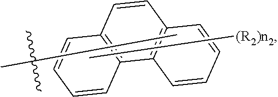

and Ar₄ is selected from the following groups:

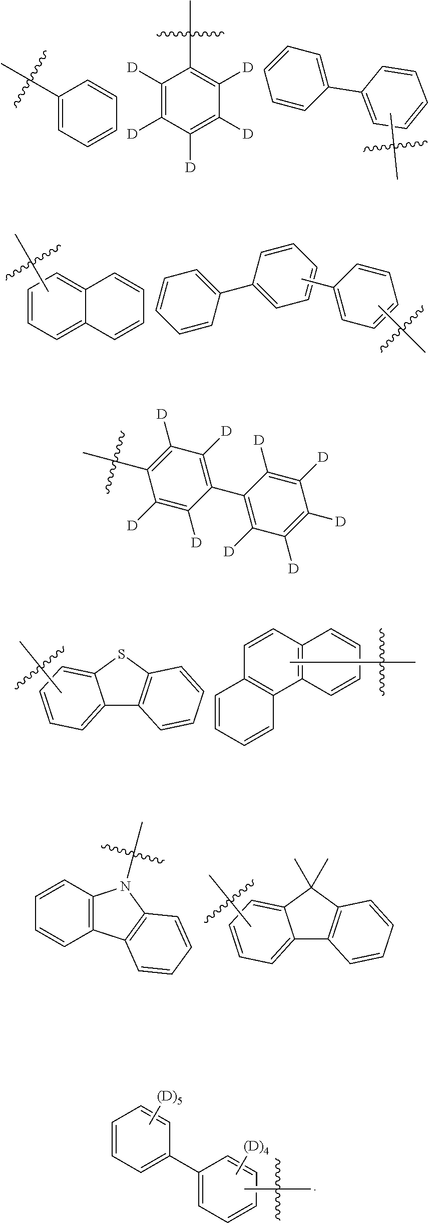

In some embodiments of the present application, Ar₄ is

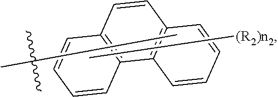

and Ar₃ is selected from the following groups:

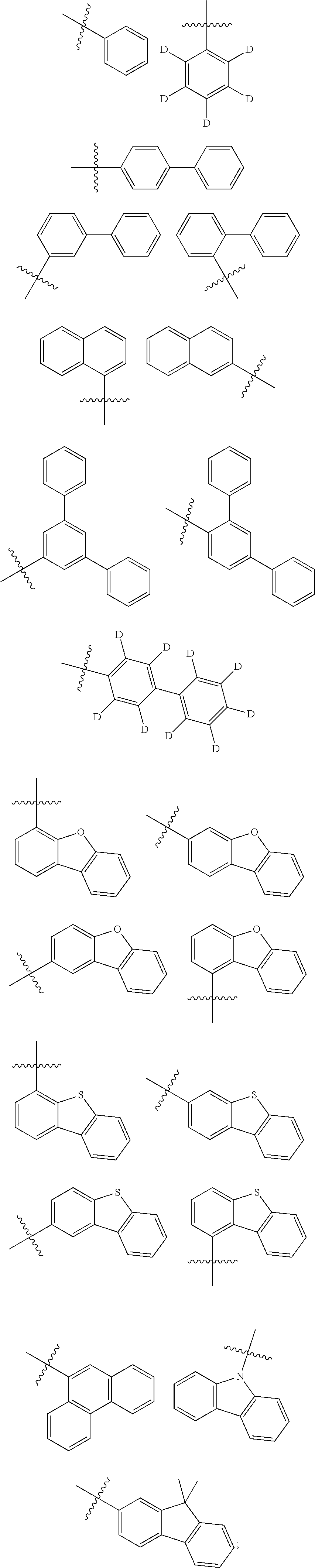

### or Ar₃ is

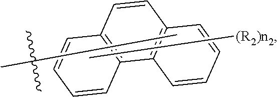

and Ar₄ is selected from the following groups:

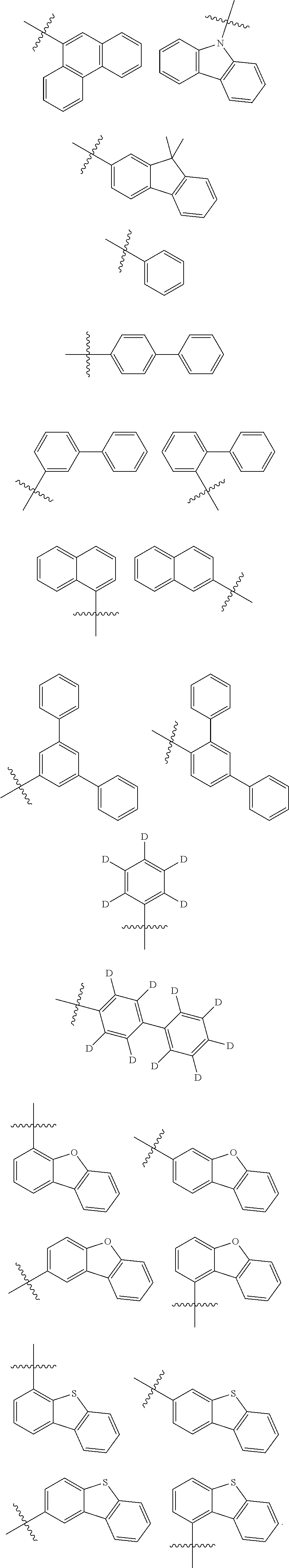

In one embodiment of the present application, L₁ is selected from a single bond, or a substituted or unsubstituted arylene having 6 to 12 carbon atoms. For example, L₁ is selected from a single bond, or a substituted or unsubstituted arylene having 6, 7, 8, 9, 10, 11, or 12 carbon atoms.

In one embodiment of the present application, L₁ is selected from a single bond, a substituted or unsubstituted phenylene, a substituted or unsubstituted naphthylene, or a substituted or unsubstituted biphenylene;

Optionally, the substituent(s) of L₁ are each independently selected from a deuterium, a fluorine, a cyano, a methyl, an ethyl, an isopropyl, a tert-butyl, or a phenyl.

In one embodiment of the present application, L₁ is selected from a single bond, or the group consisting of the following groups:

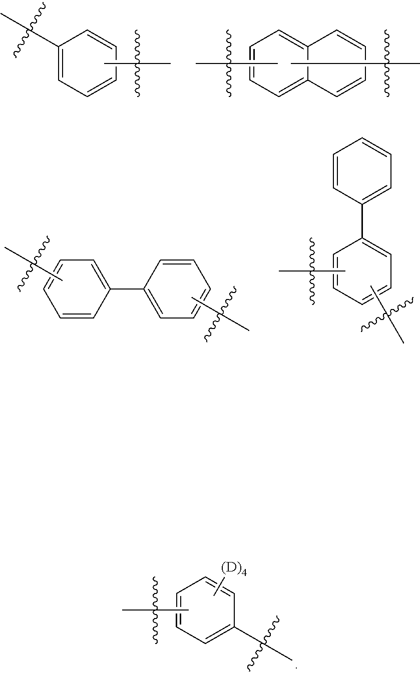

In some embodiments of the present application, L₁ is selected from a single bond, or the group consisting of the following groups:

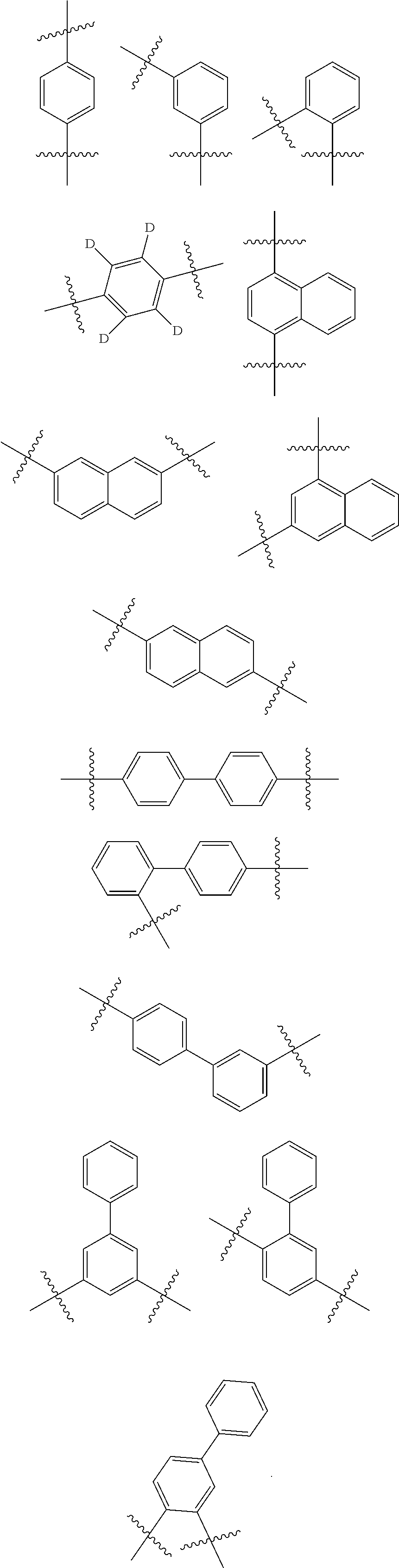

In one embodiment of the present application, L₂, L₃ and L₄ are each independently selected from a single bond, a substituted or unsubstituted arylene having 6 to 20 carbon atoms, or a substituted or unsubstituted heteroarylene having 12 to 18 carbon atoms. For example, L₂, L₃ and L₄ are each independently selected from a single bond, a substituted or unsubstituted arylene having 6, 7, 8, 9, 10, 11, 12, 13, 14, 15, 16, 17, 18, 19, or 20, or a substituted or unsubstituted heteroarylene having 12, 13, 14, 15, 16, 17, or 18 carbon atoms.

Optionally, the substituent(s) of L₂, L₃ and L₄ are each independently selected from a deuterium, a fluorine, a cyano, an alkyl having 1 to 5 carbon atoms, or an aryl having 6 to 12 carbon atoms.

Optionally, L₂, L₃ and L₄ are each independently selected from a single bond, a substituted or unsubstituted phenylene, a substituted or unsubstituted naphthylene, a substituted or unsubstituted biphenylene, a substituted or unsubstituted fluorenylene, a substituted or unsubstituted dibenzofuranylene, a substituted or unsubstituted dibenzothienylene, or a substituted or unsubstituted carbazolylene.

Optionally, the substituent(s) of L₂, L₃ and L₄ are each independently selected from a deuterium, a fluorine, a cyano, a methyl, an ethyl, an isopropyl, a tert-butyl, a phenyl, or a naphthyl.

Optionally, L₂, L₃ and L₄ are each independently selected from a single bond, or a substituted or unsubstituted group Q; wherein the unsubstituted group Q is selected from the group consisting of the following group:

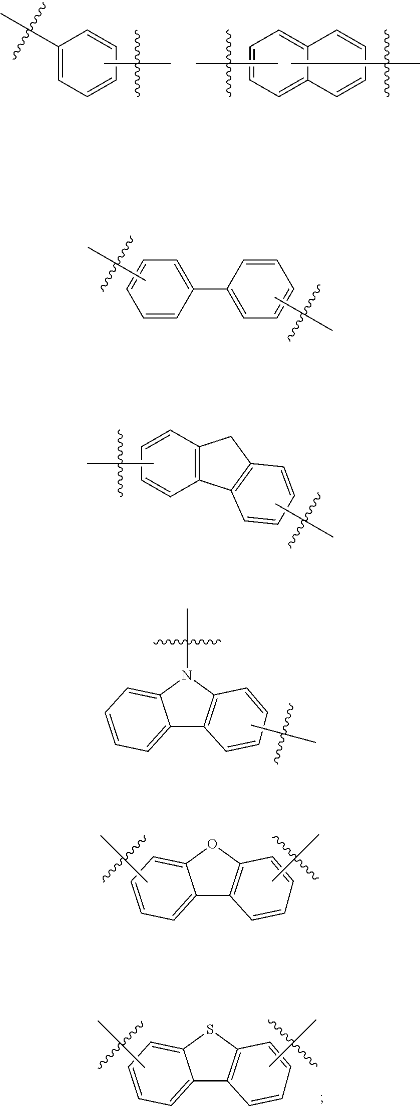

wherein, the substituted group Q has one or more substituent(s), and the substituents are each independently selected from a deuterium, a fluorine, a cyano, a methyl, an ethyl, an isopropyl, a tert-butyl, a phenyl, a naphthyl, a biphenyl, a dibenzofuranyl, a dibenzothienyl, or a carbazolyl, and when the number of the substituents is greater than 1, the substituents are each the same or different.

In some embodiments of the present application, L₂ is selected from a single bond, or the group consisting of the following groups:

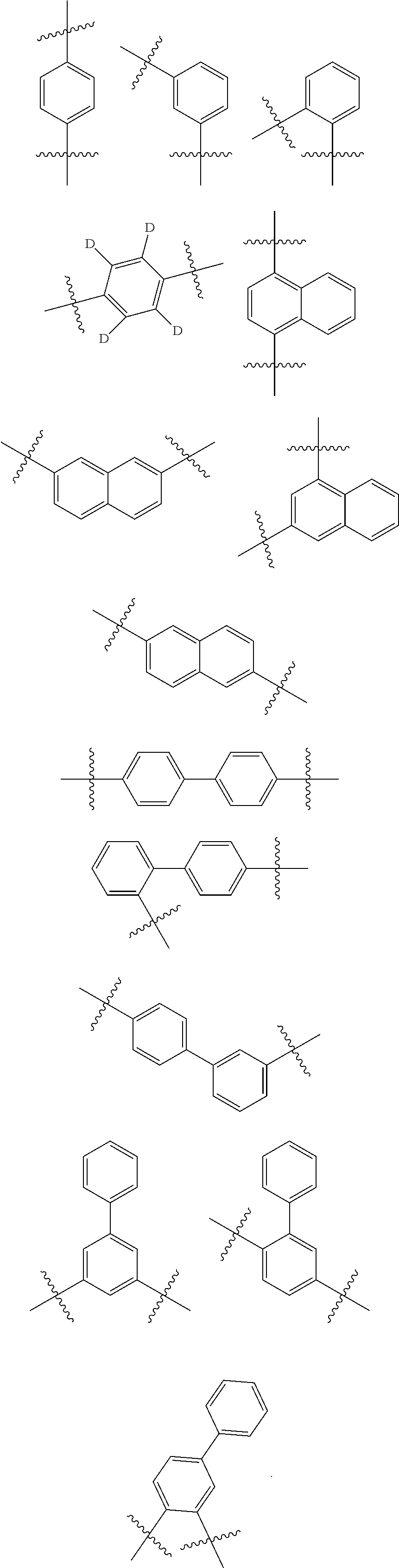

In some embodiments of the present application, L₃ and L₄ are each independently selected from a single bond, or the group consisting of the following groups:

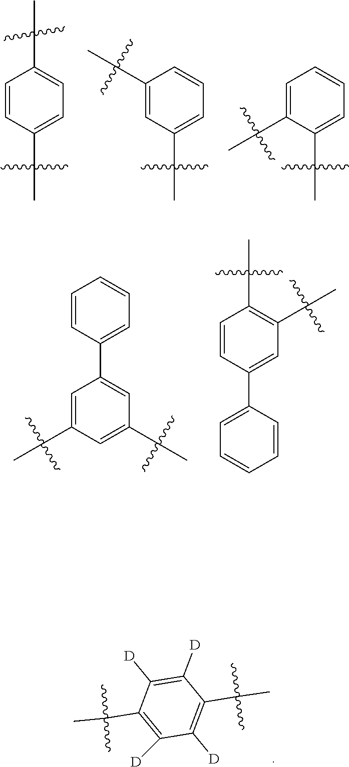 [SMILES: *c1ccc(*)cc1 ; *c1cccc(*)c1 ; *c1ccccc1* ; *c1cc(*)cc(-c2ccccc2)c1 ; *c1ccc(-c2ccccc2)cc1* ; *c1c([2H])c([2H])c(*)c([2H])c1[2H]]

In some embodiments of the present application,

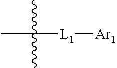

is selected from the group consisting of the following groups:

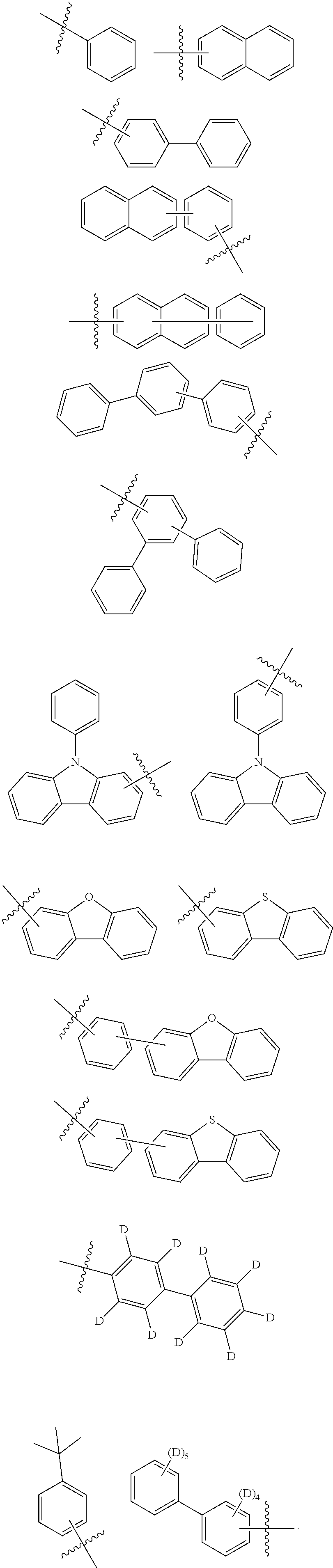

In some embodiments of the present application,

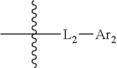

is selected from the group consisting of the following groups:

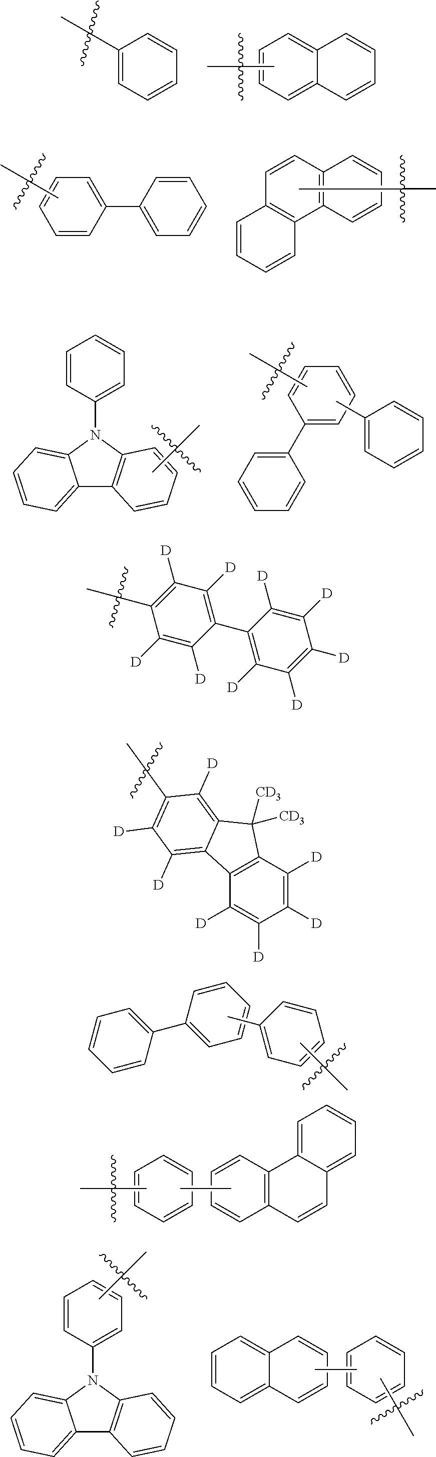

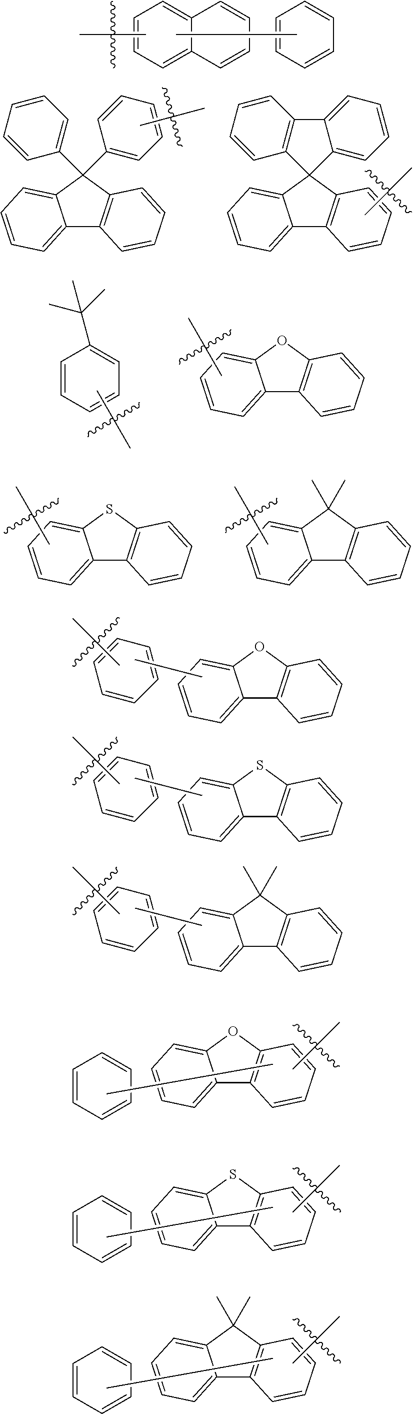

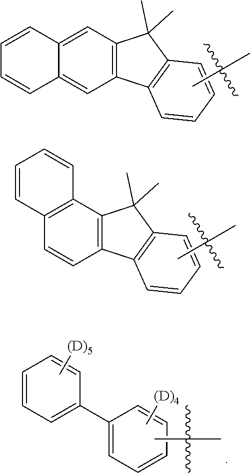

In some embodiments of the present application, R₁ and R₂ are each independently selected from a deuterium, a fluorine, a cyano, a methyl, an ethyl, an isopropyl, a tert-butyl, or a trimethylsilyl.

In some embodiments of the present application, R₁ and R₂ are each independently selected from a deuterium, a fluorine, a cyano, a methyl, an ethyl, an isopropyl, a tert-butyl, a trimethylsilyl, or a phenyl.

Optionally, the organic compound is selected from the group consisting of the following compounds:

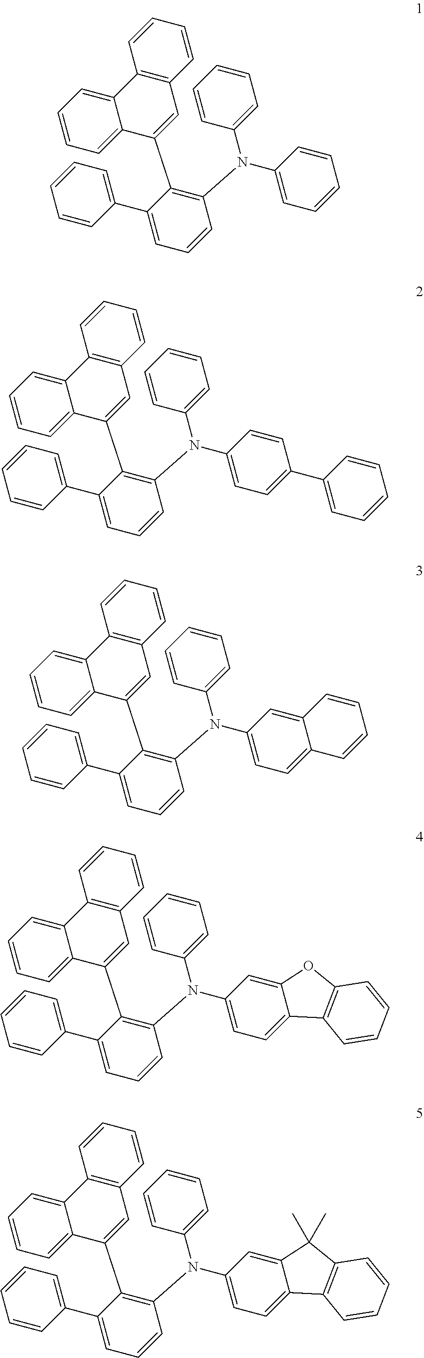 [SMILES: c1ccc(-c2cccc(N(c3ccccc3)c3ccccc3)c2-c2cc3ccccc3c3ccccc23)cc1 ; c1ccc(-c2ccc(N(c3ccccc3)c3cccc(-c4ccccc4)c3-c3cc4ccccc4c4ccccc34)cc2)cc1 ; c1ccc(-c2cccc(N(c3ccccc3)c3ccc4ccccc4c3)c2-c2cc3ccccc3c3ccccc23)cc1 ; c1ccc(-c2cccc(N(c3ccccc3)c3ccc4c(c3)oc3ccccc34)c2-c2cc3ccccc3c3ccccc23)cc1 ; CC1(C)c2ccccc2-c2ccc(N(c3ccccc3)c3cccc(-c4ccccc4)c3-c3cc4ccccc4c4ccccc34)cc21]

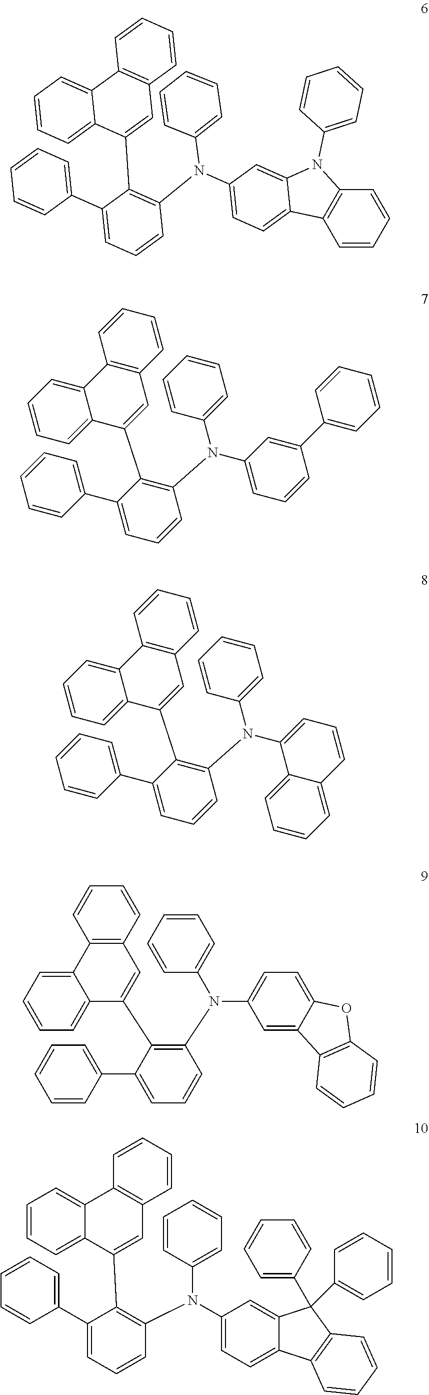

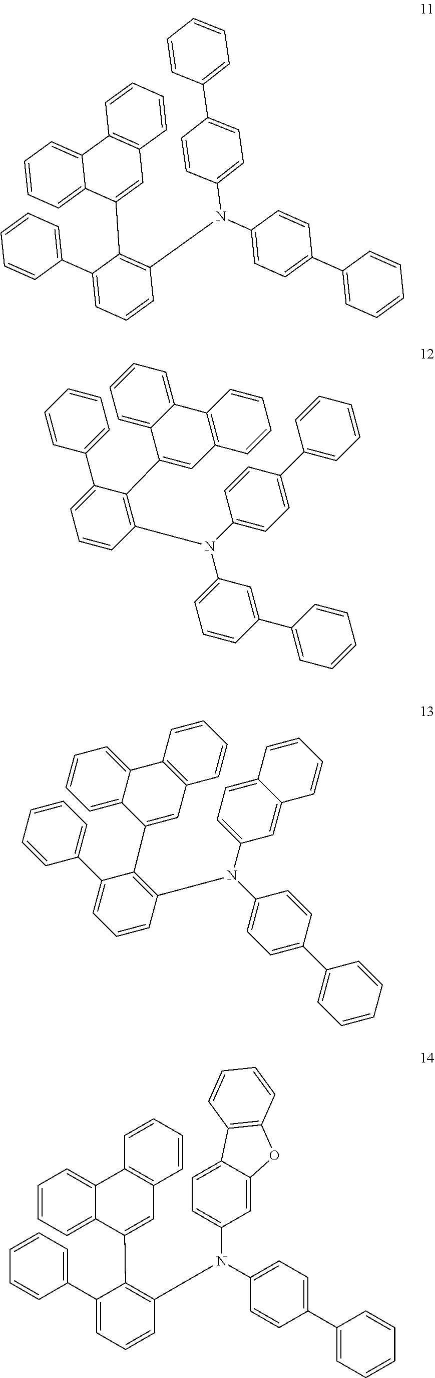

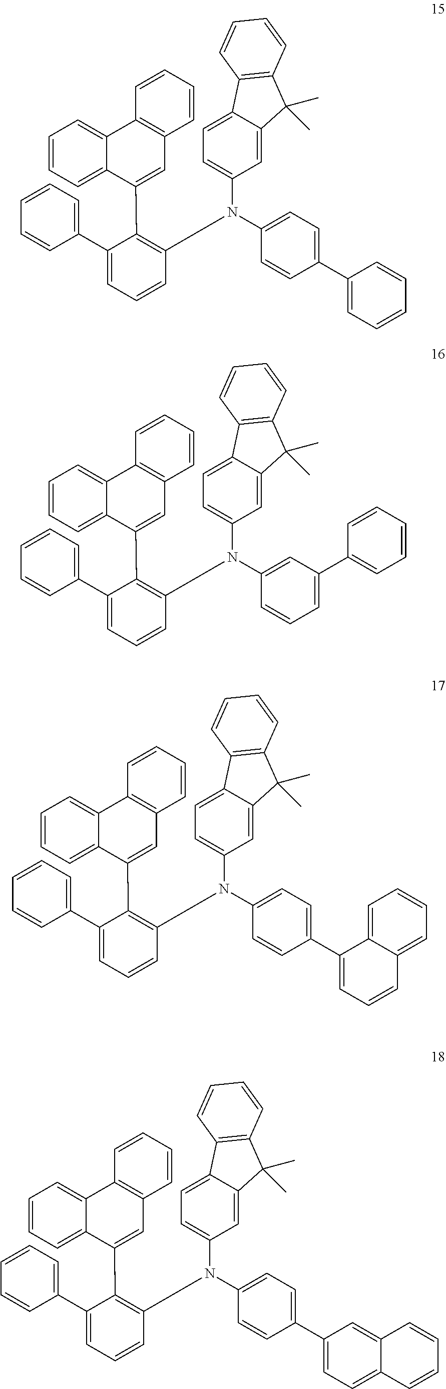 [SMILES: CC1(C)c2ccccc2-c2ccc(N(c3ccc(-c4ccccc4)cc3)c3cccc(-c4ccccc4)c3-c3cc4ccccc4c4ccccc34)cc21 ; CC1(C)c2ccccc2-c2ccc(N(c3cccc(-c4ccccc4)c3)c3cccc(-c4ccccc4)c3-c3cc4ccccc4c4ccccc34)cc21 ; CC1(C)c2ccccc2-c2ccc(N(c3ccc(-c4cccc5ccccc45)cc3)c3cccc(-c4ccccc4)c3-c3cc4ccccc4c4ccccc34)cc21 ; CC1(C)c2ccccc2-c2ccc(N(c3ccc(-c4ccc5ccccc5c4)cc3)c3cccc(-c4ccccc4)c3-c3cc4ccccc4c4ccccc34)cc21]

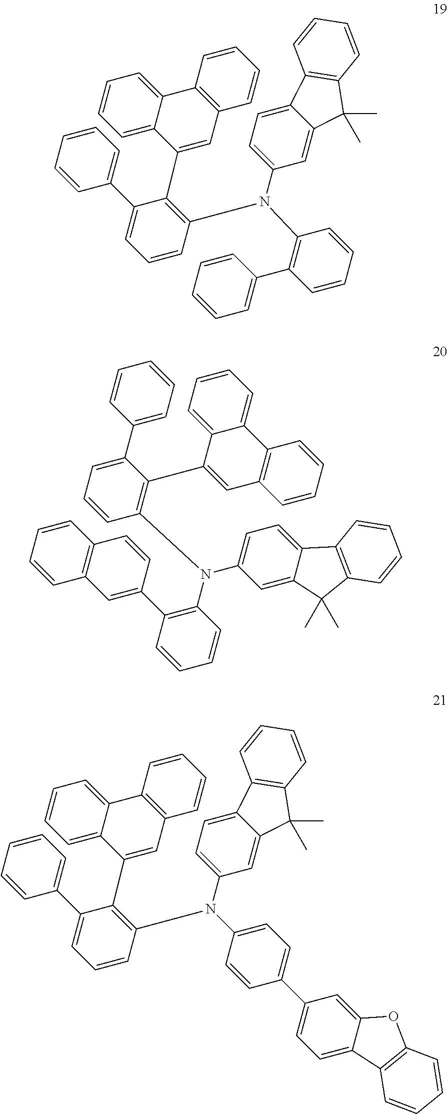 [SMILES: CC1(C)c2ccccc2-c2ccc(N(c3ccccc3-c3ccccc3)c3cccc(-c4ccccc4)c3-c3cc4ccccc4c4ccccc34)cc21 ; CC1(C)c2ccccc2-c2ccc(N(c3ccccc3-c3ccc4ccccc4c3)c3cccc(-c4ccccc4)c3-c3cc4ccccc4c4ccccc34)cc21 ; CC1(C)c2ccccc2-c2ccc(N(c3ccc(-c4ccc5c(c4)oc4ccccc45)cc3)c3cccc(-c4ccccc4)c3-c3cc4ccccc4c4ccccc34)cc21]

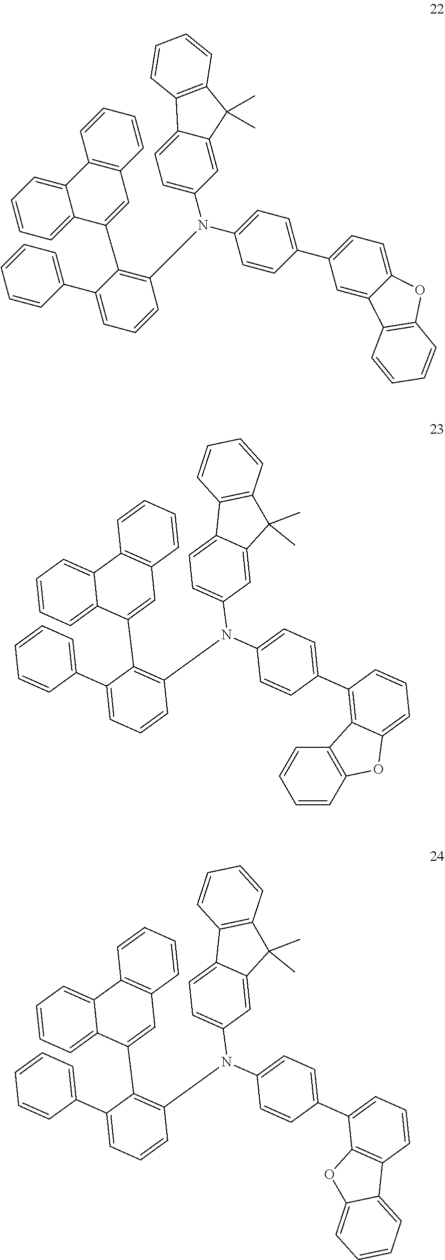 [SMILES: CC1(C)c2ccccc2-c2ccc(N(c3ccc(-c4ccc5oc6ccccc6c5c4)cc3)c3cccc(-c4ccccc4)c3-c3cc4ccccc4c4ccccc34)cc21 ; CC1(C)c2ccccc2-c2ccc(N(c3ccc(-c4cccc5oc6ccccc6c45)cc3)c3cccc(-c4ccccc4)c3-c3cc4ccccc4c4ccccc34)cc21 ; CC1(C)c2ccccc2-c2ccc(N(c3ccc(-c4cccc5c4oc4ccccc45)cc3)c3cccc(-c4ccccc4)c3-c3cc4ccccc4c4ccccc34)cc21]

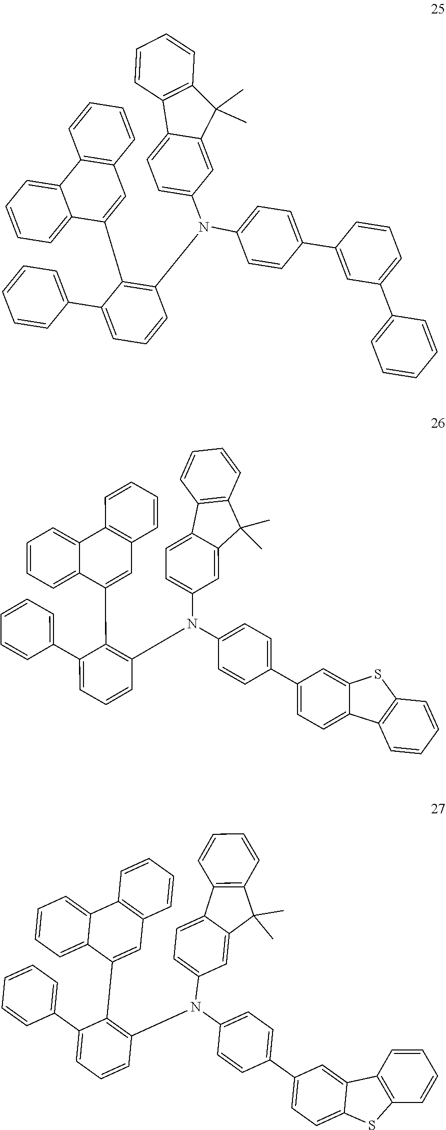 [SMILES: CC1(C)c2ccccc2-c2ccc(N(c3ccc(-c4cccc(-c5ccccc5)c4)cc3)c3cccc(-c4ccccc4)c3-c3cc4ccccc4c4ccccc34)cc21 ; CC1(C)c2ccccc2-c2ccc(N(c3ccc(-c4ccc5c(c4)sc4ccccc45)cc3)c3cccc(-c4ccccc4)c3-c3cc4ccccc4c4ccccc34)cc21 ; CC1(C)c2ccccc2-c2ccc(N(c3ccc(-c4ccc5sc6ccccc6c5c4)cc3)c3cccc(-c4ccccc4)c3-c3cc4ccccc4c4ccccc34)cc21]

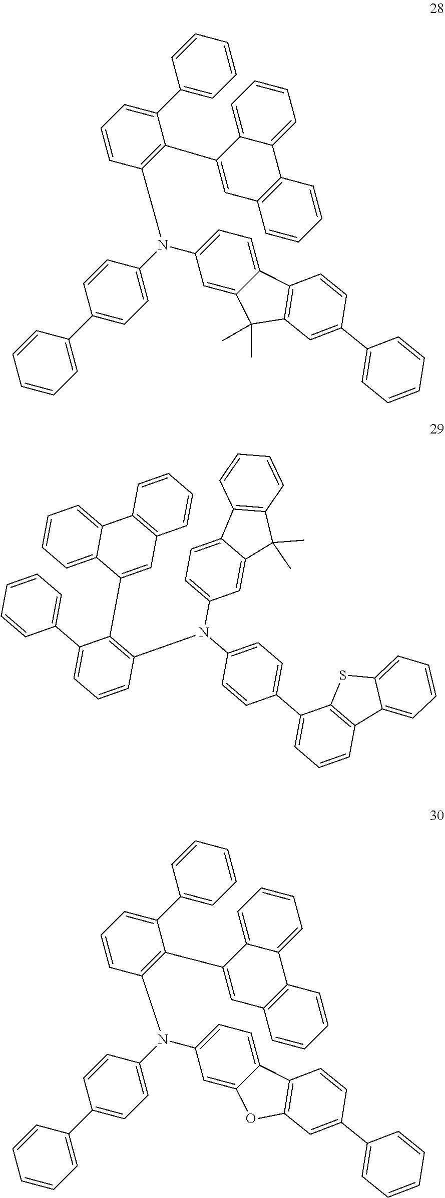 [SMILES: CC1(C)c2cc(-c3ccccc3)ccc2-c2ccc(N(c3ccc(-c4ccccc4)cc3)c3cccc(-c4ccccc4)c3-c3cc4ccccc4c4ccccc34)cc21 ; CC1(C)c2ccccc2-c2ccc(N(c3ccc(-c4cccc5c4sc4ccccc45)cc3)c3cccc(-c4ccccc4)c3-c3cc4ccccc4c4ccccc34)cc21 ; c1ccc(-c2ccc(N(c3ccc4c(c3)oc3cc(-c5ccccc5)ccc34)c3cccc(-c4ccccc4)c3-c3cc4ccccc4c4ccccc34)cc2)cc1]

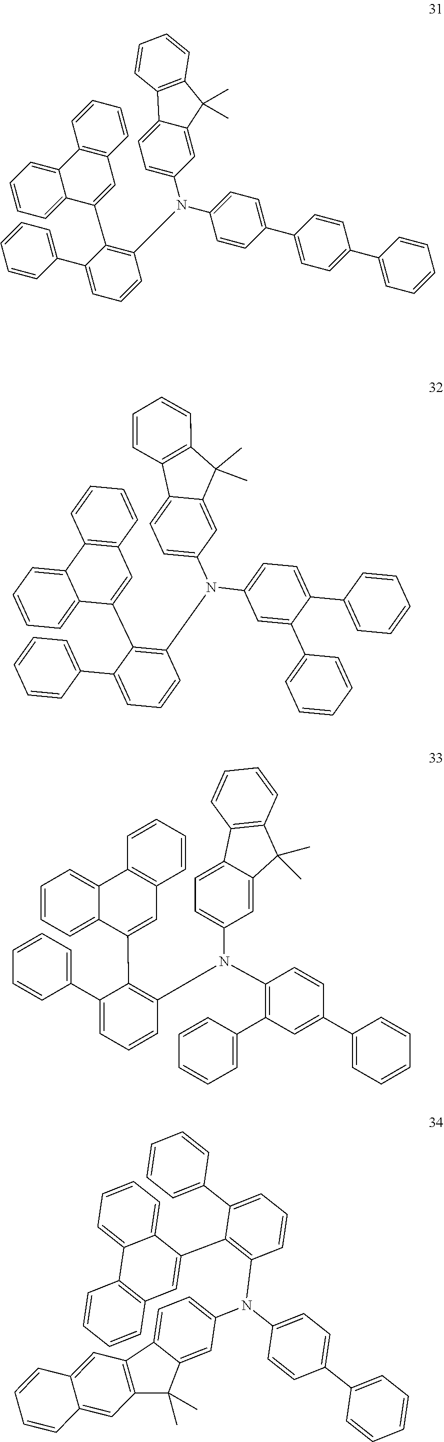 [SMILES: CC1(C)c2ccccc2-c2ccc(N(c3ccc(-c4ccc(-c5ccccc5)cc4)cc3)c3cccc(-c4ccccc4)c3-c3cc4ccccc4c4ccccc34)cc21 ; CC1(C)c2ccccc2-c2ccc(N(c3ccc(-c4ccccc4)c(-c4ccccc4)c3)c3cccc(-c4ccccc4)c3-c3cc4ccccc4c4ccccc34)cc21 ; CC1(C)c2ccccc2-c2ccc(N(c3ccc(-c4ccccc4)cc3-c3ccccc3)c3cccc(-c4ccccc4)c3-c3cc4ccccc4c4ccccc34)cc21 ; CC1(C)c2cc(N(c3ccc(-c4ccccc4)cc3)c3cccc(-c4ccccc4)c3-c3cc4ccccc4c4ccccc34)ccc2-c2cc3ccccc3cc21]

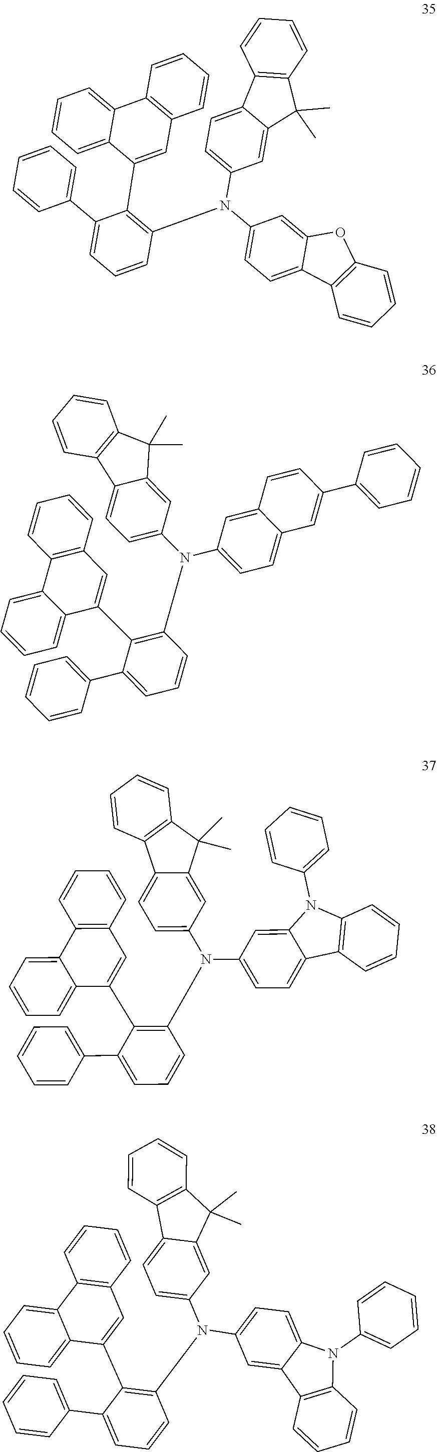 [SMILES: CC1(C)c2ccccc2-c2ccc(N(c3ccc4c(c3)oc3ccccc34)c3cccc(-c4ccccc4)c3-c3cc4ccccc4c4ccccc34)cc21 ; CC1(C)c2ccccc2-c2ccc(N(c3ccc4cc(-c5ccccc5)ccc4c3)c3cccc(-c4ccccc4)c3-c3cc4ccccc4c4ccccc34)cc21 ; CC1(C)c2ccccc2-c2ccc(N(c3ccc4c5ccccc5n(-c5ccccc5)c4c3)c3cccc(-c4ccccc4)c3-c3cc4ccccc4c4ccccc34)cc21 ; CC1(C)c2ccccc2-c2ccc(N(c3ccc4c(c3)c3ccccc3n4-c3ccccc3)c3cccc(-c4ccccc4)c3-c3cc4ccccc4c4ccccc34)cc21]

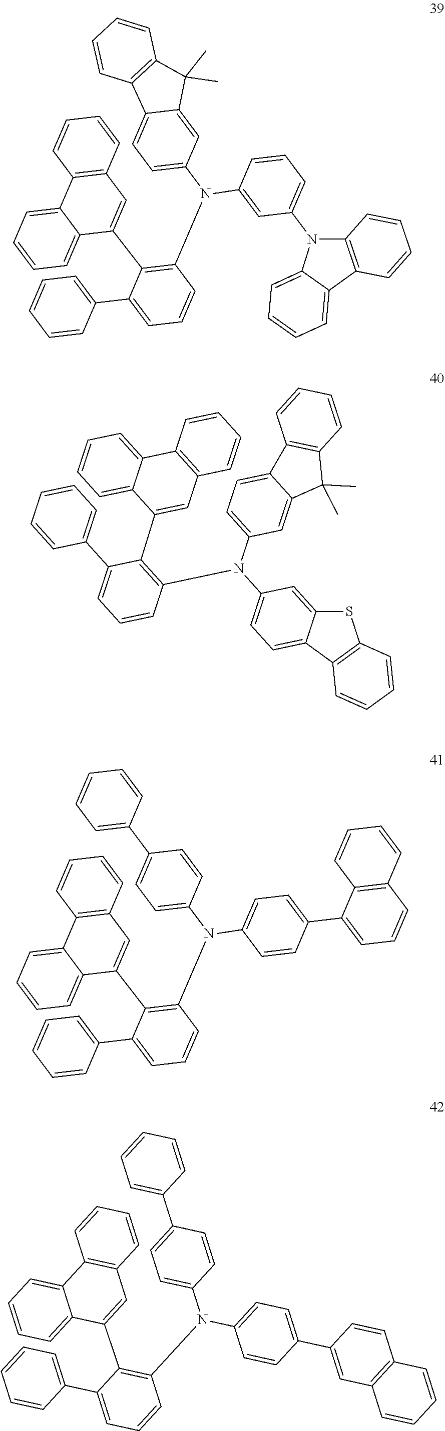 [SMILES: CC1(C)c2ccccc2-c2ccc(N(c3cccc(-n4c5ccccc5c5ccccc54)c3)c3cccc(-c4ccccc4)c3-c3cc4ccccc4c4ccccc34)cc21 ; CC1(C)c2ccccc2-c2ccc(N(c3ccc4c(c3)sc3ccccc34)c3cccc(-c4ccccc4)c3-c3cc4ccccc4c4ccccc34)cc21 ; c1ccc(-c2ccc(N(c3ccc(-c4cccc5ccccc45)cc3)c3cccc(-c4ccccc4)c3-c3cc4ccccc4c4ccccc34)cc2)cc1 ; c1ccc(-c2ccc(N(c3ccc(-c4ccc5ccccc5c4)cc3)c3cccc(-c4ccccc4)c3-c3cc4ccccc4c4ccccc34)cc2)cc1]

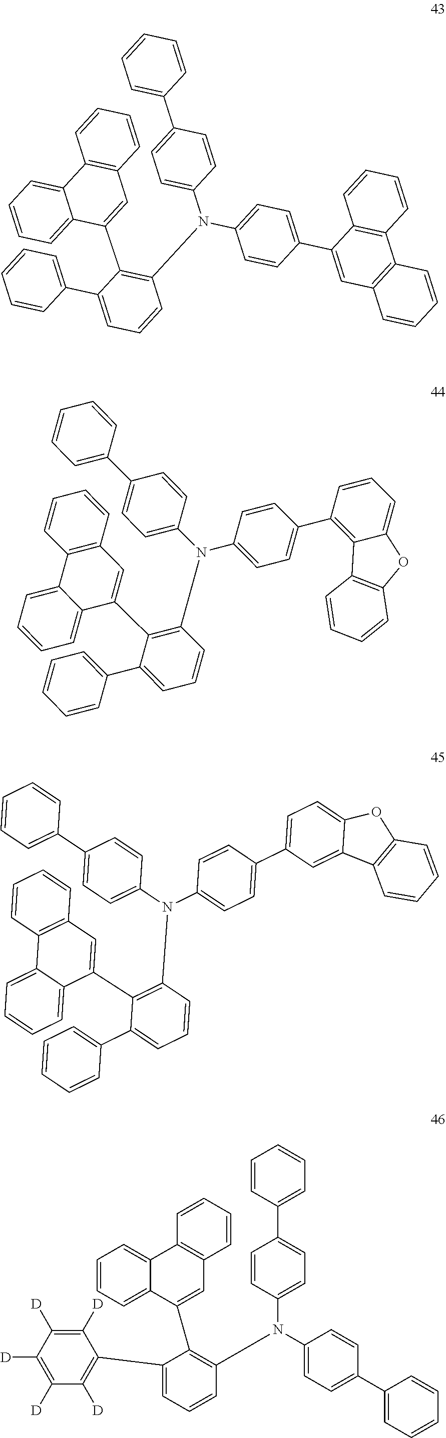 [SMILES: c1ccc(-c2ccc(N(c3ccc(-c4cc5ccccc5c5ccccc45)cc3)c3cccc(-c4ccccc4)c3-c3cc4ccccc4c4ccccc34)cc2)cc1 ; c1ccc(-c2ccc(N(c3ccc(-c4cccc5oc6ccccc6c45)cc3)c3cccc(-c4ccccc4)c3-c3cc4ccccc4c4ccccc34)cc2)cc1 ; c1ccc(-c2ccc(N(c3ccc(-c4ccc5oc6ccccc6c5c4)cc3)c3cccc(-c4ccccc4)c3-c3cc4ccccc4c4ccccc34)cc2)cc1 ; [2H]c1c([2H])c([2H])c(-c2cccc(N(c3ccc(-c4ccccc4)cc3)c3ccc(-c4ccccc4)cc3)c2-c2cc3ccccc3c3ccccc23)c([2H])c1[2H]]

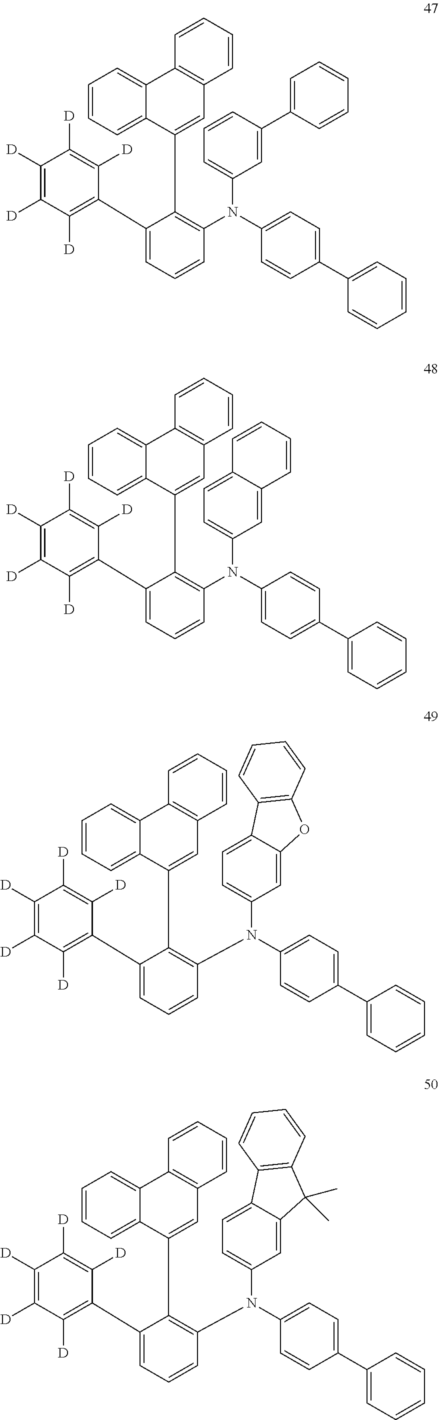 [SMILES: [2H]c1c([2H])c([2H])c(-c2cccc(N(c3ccc(-c4ccccc4)cc3)c3cccc(-c4ccccc4)c3)c2-c2cc3ccccc3c3ccccc23)c([2H])c1[2H] ; [2H]c1c([2H])c([2H])c(-c2cccc(N(c3ccc(-c4ccccc4)cc3)c3ccc4ccccc4c3)c2-c2cc3ccccc3c3ccccc23)c([2H])c1[2H] ; [2H]c1c([2H])c([2H])c(-c2cccc(N(c3ccc(-c4ccccc4)cc3)c3ccc4c(c3)oc3ccccc34)c2-c2cc3ccccc3c3ccccc23)c([2H])c1[2H] ; [2H]c1c([2H])c([2H])c(-c2cccc(N(c3ccc(-c4ccccc4)cc3)c3ccc4c(c3)C(C)(C)c3ccccc3-4)c2-c2cc3ccccc3c3ccccc23)c([2H])c1[2H]]

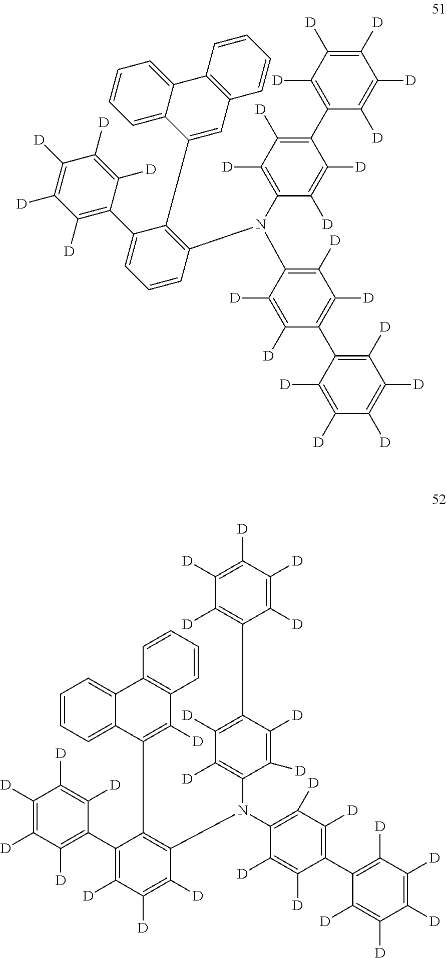 [SMILES: [2H]c1c([2H])c([2H])c(-c2cccc(N(c3c([2H])c([2H])c(-c4c([2H])c([2H])c([2H])c([2H])c4[2H])c([2H])c3[2H])c3c([2H])c([2H])c(-c4c([2H])c([2H])c([2H])c([2H])c4[2H])c([2H])c3[2H])c2-c2cc3ccccc3c3ccccc23)c([2H])c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c([2H])c(-c4c([2H])c([2H])c([2H])c([2H])c4[2H])c3-c3c([2H])c4ccccc4c4ccccc34)c([2H])c2[2H])c([2H])c1[2H]]

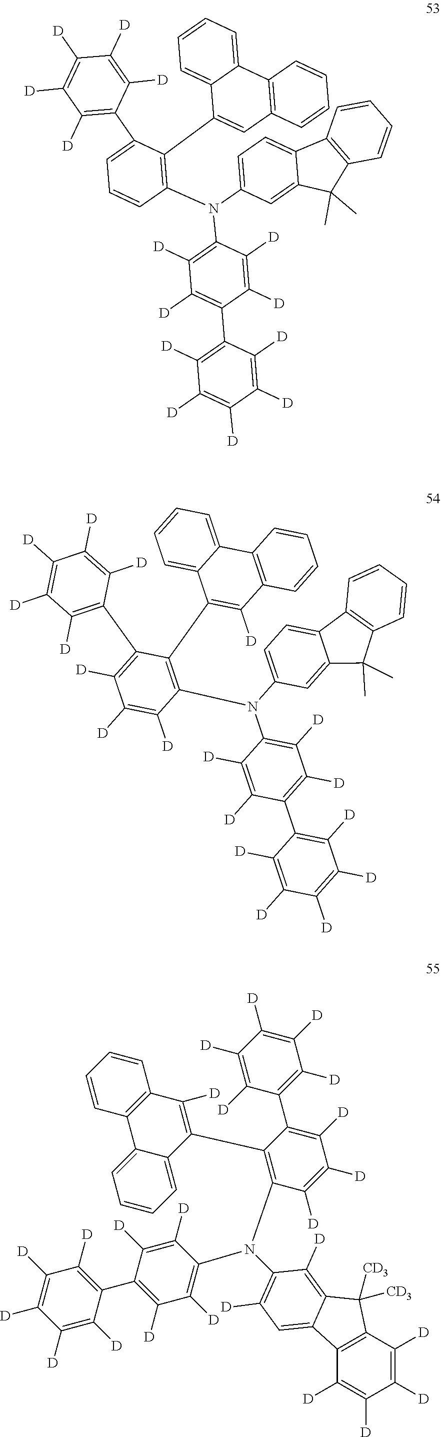

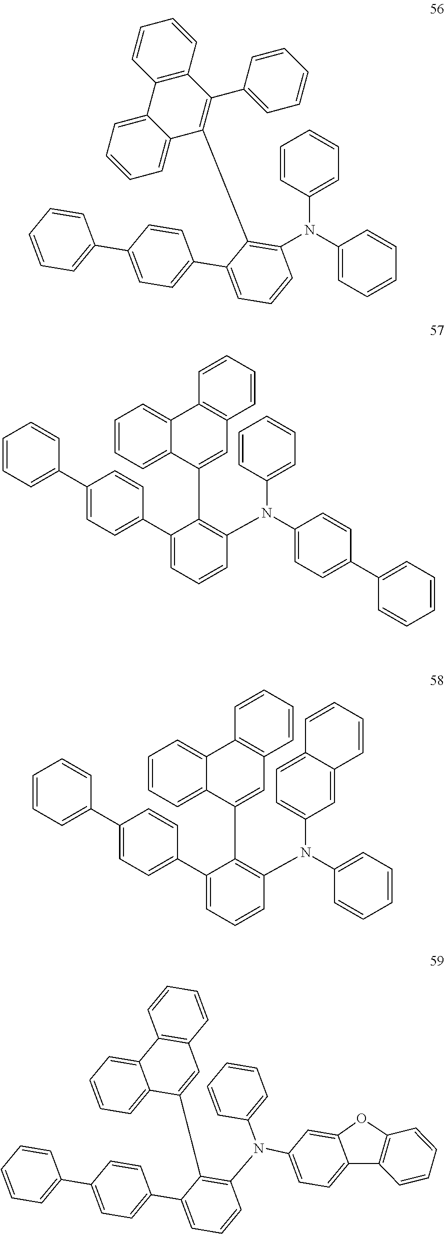 [SMILES: c1ccc(-c2ccc(-c3cccc(N(c4ccccc4)c4ccccc4)c3-c3c(-c4ccccc4)c4ccccc4c4ccccc34)cc2)cc1 ; c1ccc(-c2ccc(-c3cccc(N(c4ccccc4)c4ccc(-c5ccccc5)cc4)c3-c3cc4ccccc4c4ccccc34)cc2)cc1 ; c1ccc(-c2ccc(-c3cccc(N(c4ccccc4)c4ccc5ccccc5c4)c3-c3cc4ccccc4c4ccccc34)cc2)cc1 ; c1ccc(-c2ccc(-c3cccc(N(c4ccccc4)c4ccc5c(c4)oc4ccccc45)c3-c3cc4ccccc4c4ccccc34)cc2)cc1]

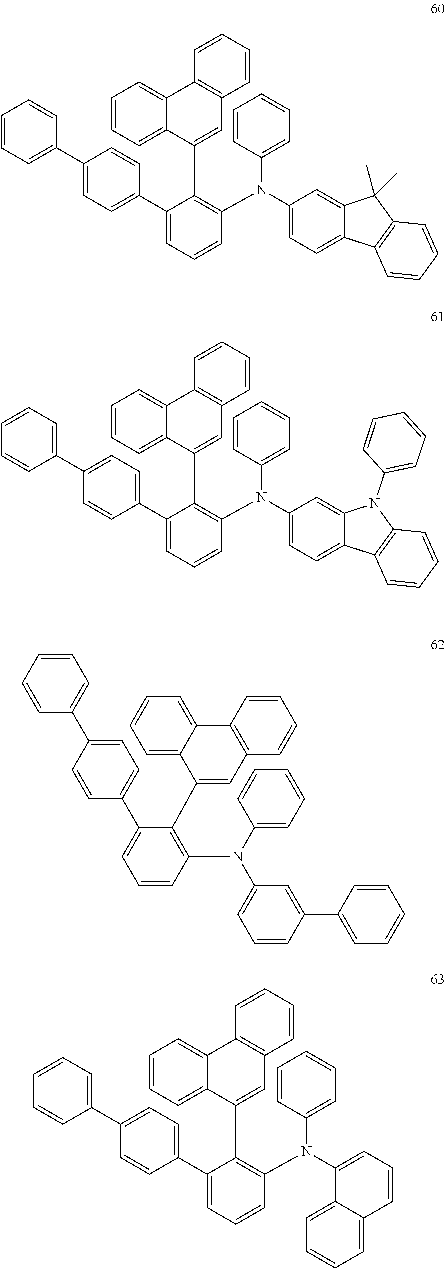 [SMILES: CC1(C)c2ccccc2-c2ccc(N(c3ccccc3)c3cccc(-c4ccc(-c5ccccc5)cc4)c3-c3cc4ccccc4c4ccccc34)cc21 ; c1ccc(-c2ccc(-c3cccc(N(c4ccccc4)c4ccc5c6ccccc6n(-c6ccccc6)c5c4)c3-c3cc4ccccc4c4ccccc34)cc2)cc1 ; c1ccc(-c2ccc(-c3cccc(N(c4ccccc4)c4cccc(-c5ccccc5)c4)c3-c3cc4ccccc4c4ccccc34)cc2)cc1 ; c1ccc(-c2ccc(-c3cccc(N(c4ccccc4)c4cccc5ccccc45)c3-c3cc4ccccc4c4ccccc34)cc2)cc1]

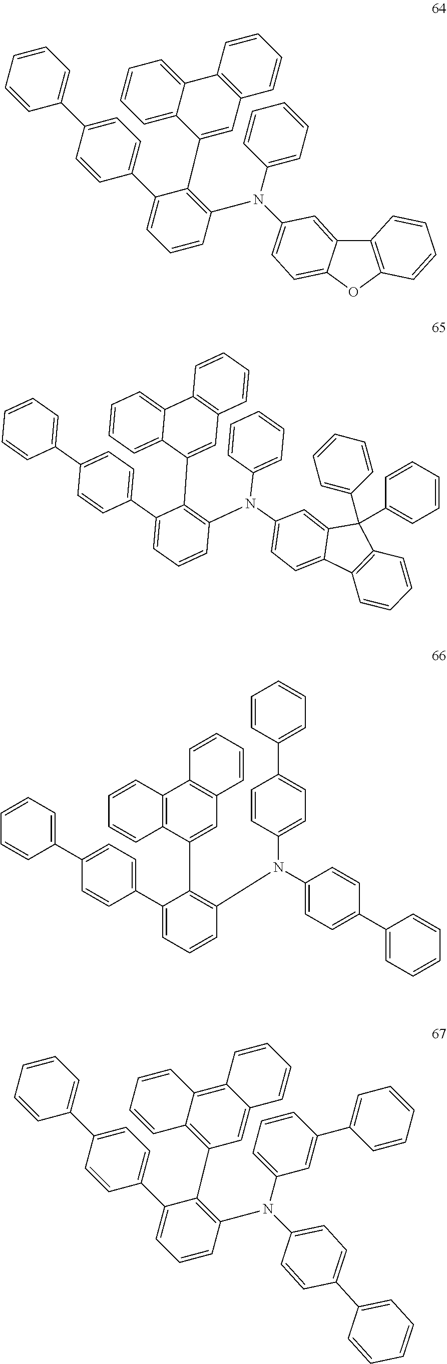 [SMILES: c1ccc(-c2ccc(-c3cccc(N(c4ccccc4)c4ccc5oc6ccccc6c5c4)c3-c3cc4ccccc4c4ccccc34)cc2)cc1 ; c1ccc(-c2ccc(-c3cccc(N(c4ccccc4)c4ccc5c(c4)C(c4ccccc4)(c4ccccc4)c4ccccc4-5)c3-c3cc4ccccc4c4ccccc34)cc2)cc1 ; c1ccc(-c2ccc(-c3cccc(N(c4ccc(-c5ccccc5)cc4)c4ccc(-c5ccccc5)cc4)c3-c3cc4ccccc4c4ccccc34)cc2)cc1 ; c1ccc(-c2ccc(-c3cccc(N(c4ccc(-c5ccccc5)cc4)c4cccc(-c5ccccc5)c4)c3-c3cc4ccccc4c4ccccc34)cc2)cc1]

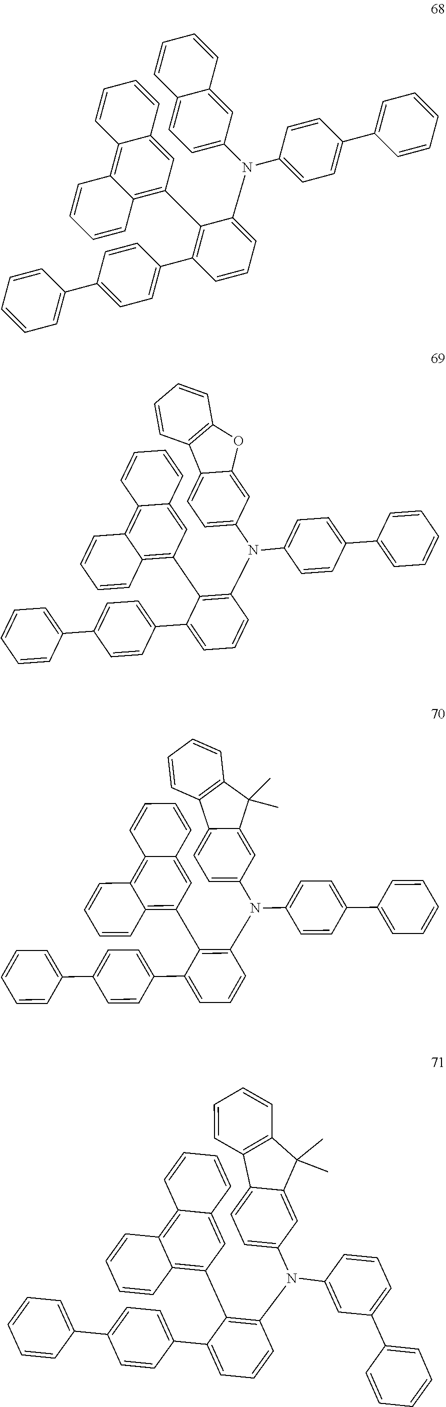 [SMILES: c1ccc(-c2ccc(-c3cccc(N(c4ccc(-c5ccccc5)cc4)c4ccc5ccccc5c4)c3-c3cc4ccccc4c4ccccc34)cc2)cc1 ; c1ccc(-c2ccc(-c3cccc(N(c4ccc(-c5ccccc5)cc4)c4ccc5c(c4)oc4ccccc45)c3-c3cc4ccccc4c4ccccc34)cc2)cc1 ; CC1(C)c2ccccc2-c2ccc(N(c3ccc(-c4ccccc4)cc3)c3cccc(-c4ccc(-c5ccccc5)cc4)c3-c3cc4ccccc4c4ccccc34)cc21 ; CC1(C)c2ccccc2-c2ccc(N(c3cccc(-c4ccccc4)c3)c3cccc(-c4ccc(-c5ccccc5)cc4)c3-c3cc4ccccc4c4ccccc34)cc21]

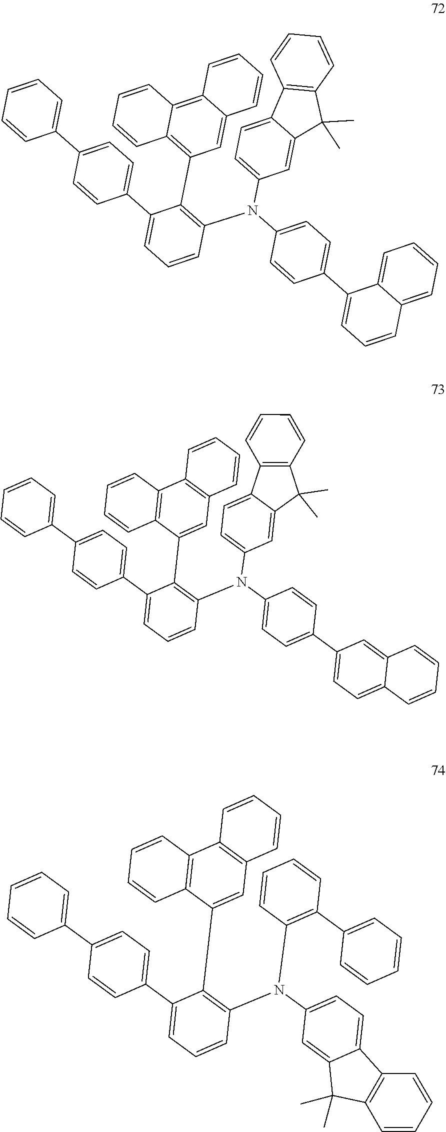 [SMILES: CC1(C)c2ccccc2-c2ccc(N(c3ccc(-c4cccc5ccccc45)cc3)c3cccc(-c4ccc(-c5ccccc5)cc4)c3-c3cc4ccccc4c4ccccc34)cc21 ; CC1(C)c2ccccc2-c2ccc(N(c3ccc(-c4ccc5ccccc5c4)cc3)c3cccc(-c4ccc(-c5ccccc5)cc4)c3-c3cc4ccccc4c4ccccc34)cc21 ; CC1(C)c2ccccc2-c2ccc(N(c3ccccc3-c3ccccc3)c3cccc(-c4ccc(-c5ccccc5)cc4)c3-c3cc4ccccc4c4ccccc34)cc21]

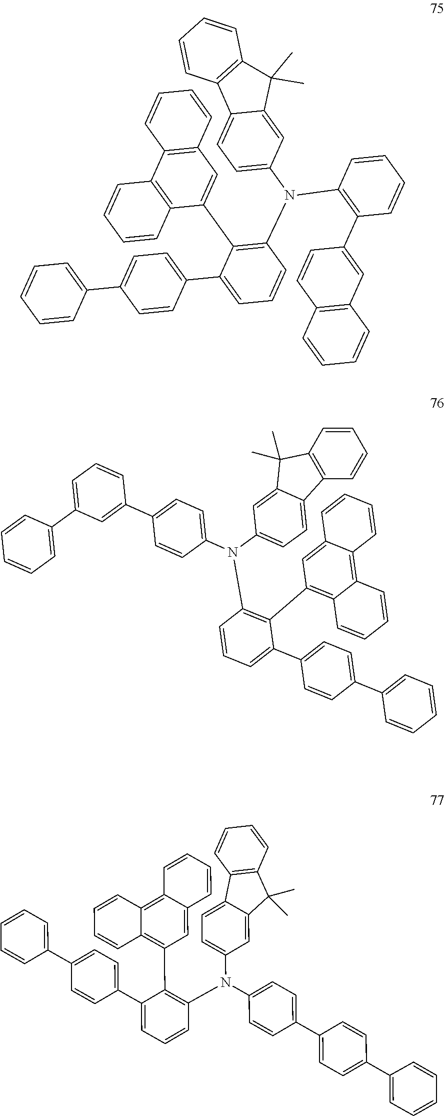 [SMILES: CC1(C)c2ccccc2-c2ccc(N(c3ccccc3-c3ccc4ccccc4c3)c3cccc(-c4ccc(-c5ccccc5)cc4)c3-c3cc4ccccc4c4ccccc34)cc21 ; CC1(C)c2ccccc2-c2ccc(N(c3ccc(-c4cccc(-c5ccccc5)c4)cc3)c3cccc(-c4ccc(-c5ccccc5)cc4)c3-c3cc4ccccc4c4ccccc34)cc21 ; CC1(C)c2ccccc2-c2ccc(N(c3ccc(-c4ccc(-c5ccccc5)cc4)cc3)c3cccc(-c4ccc(-c5ccccc5)cc4)c3-c3cc4ccccc4c4ccccc34)cc21]

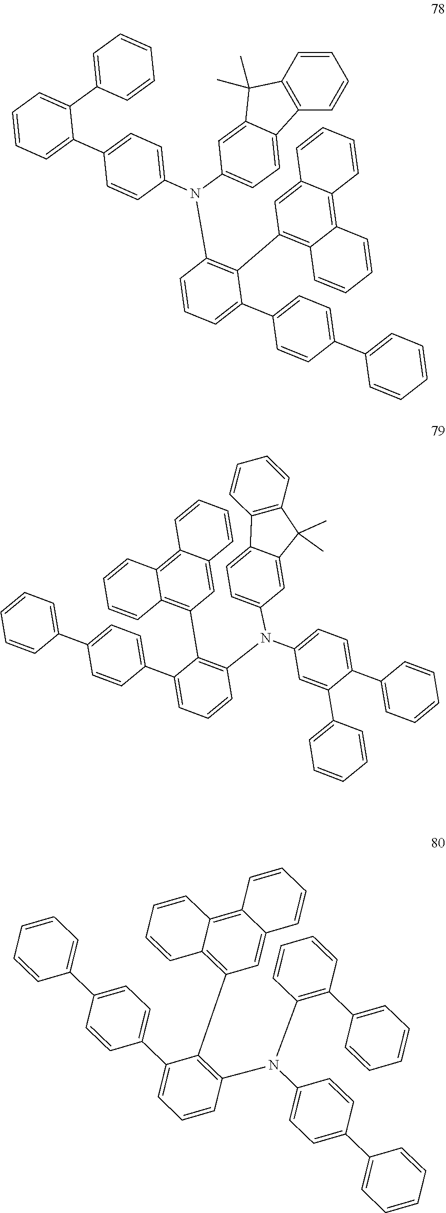 [SMILES: CC1(C)c2ccccc2-c2ccc(N(c3ccc(-c4ccccc4-c4ccccc4)cc3)c3cccc(-c4ccc(-c5ccccc5)cc4)c3-c3cc4ccccc4c4ccccc34)cc21 ; CC1(C)c2ccccc2-c2ccc(N(c3ccc(-c4ccccc4)c(-c4ccccc4)c3)c3cccc(-c4ccc(-c5ccccc5)cc4)c3-c3cc4ccccc4c4ccccc34)cc21 ; c1ccc(-c2ccc(-c3cccc(N(c4ccc(-c5ccccc5)cc4)c4ccccc4-c4ccccc4)c3-c3cc4ccccc4c4ccccc34)cc2)cc1]

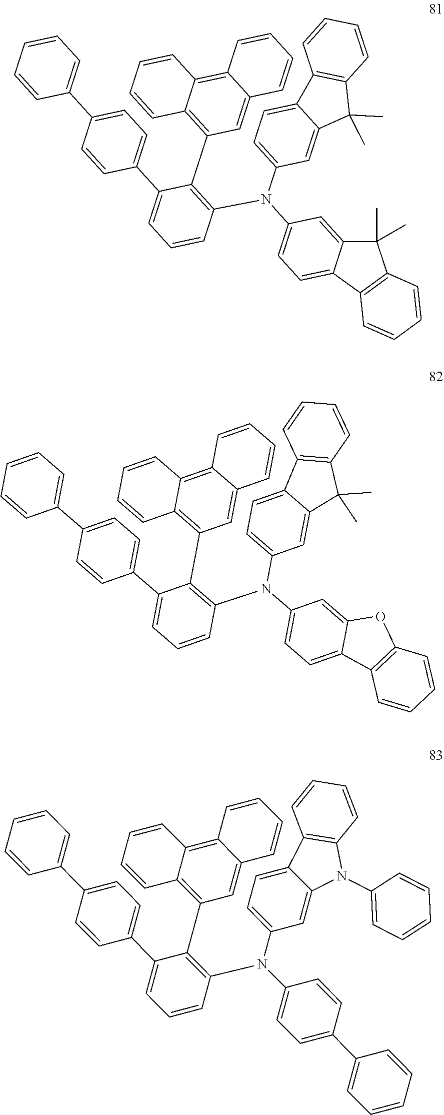 [SMILES: CC1(C)c2ccccc2-c2ccc(N(c3ccc4c(c3)C(C)(C)c3ccccc3-4)c3cccc(-c4ccc(-c5ccccc5)cc4)c3-c3cc4ccccc4c4ccccc34)cc21 ; CC1(C)c2ccccc2-c2ccc(N(c3ccc4c(c3)oc3ccccc34)c3cccc(-c4ccc(-c5ccccc5)cc4)c3-c3cc4ccccc4c4ccccc34)cc21 ; c1ccc(-c2ccc(-c3cccc(N(c4ccc(-c5ccccc5)cc4)c4ccc5c6ccccc6n(-c6ccccc6)c5c4)c3-c3cc4ccccc4c4ccccc34)cc2)cc1]

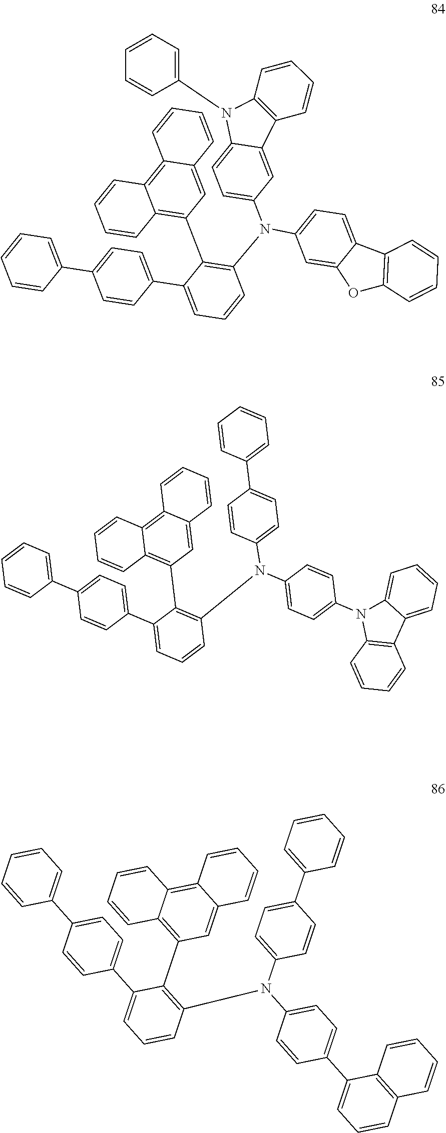 [SMILES: c1ccc(-c2ccc(-c3cccc(N(c4ccc5c(c4)oc4ccccc45)c4ccc5c(c4)c4ccccc4n5-c4ccccc4)c3-c3cc4ccccc4c4ccccc34)cc2)cc1 ; c1ccc(-c2ccc(-c3cccc(N(c4ccc(-c5ccccc5)cc4)c4ccc(-n5c6ccccc6c6ccccc65)cc4)c3-c3cc4ccccc4c4ccccc34)cc2)cc1 ; c1ccc(-c2ccc(-c3cccc(N(c4ccc(-c5ccccc5)cc4)c4ccc(-c5cccc6ccccc56)cc4)c3-c3cc4ccccc4c4ccccc34)cc2)cc1]

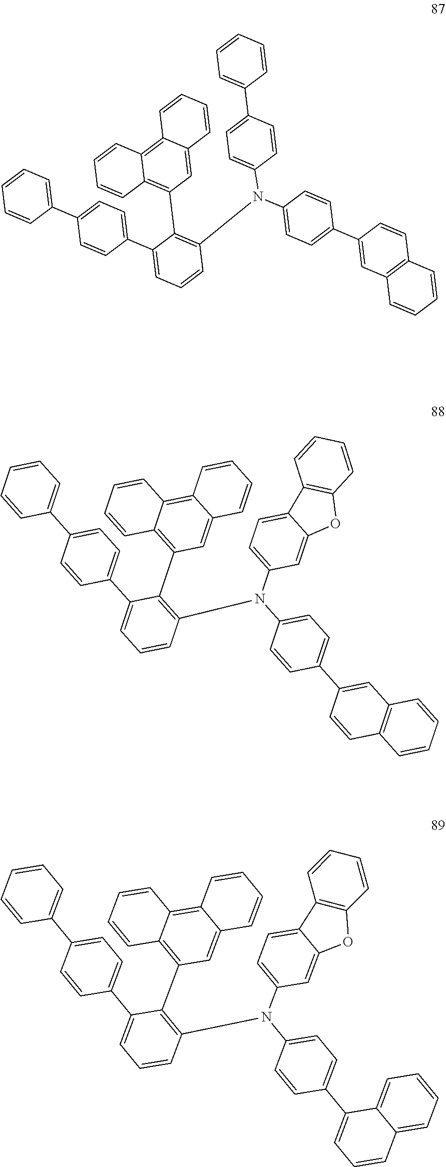 [SMILES: c1ccc(-c2ccc(-c3cccc(N(c4ccc(-c5ccccc5)cc4)c4ccc(-c5ccc6ccccc6c5)cc4)c3-c3cc4ccccc4c4ccccc34)cc2)cc1 ; c1ccc(-c2ccc(-c3cccc(N(c4ccc(-c5ccc6ccccc6c5)cc4)c4ccc5c(c4)oc4ccccc45)c3-c3cc4ccccc4c4ccccc34)cc2)cc1 ; c1ccc(-c2ccc(-c3cccc(N(c4ccc(-c5cccc6ccccc56)cc4)c4ccc5c(c4)oc4ccccc45)c3-c3cc4ccccc4c4ccccc34)cc2)cc1]

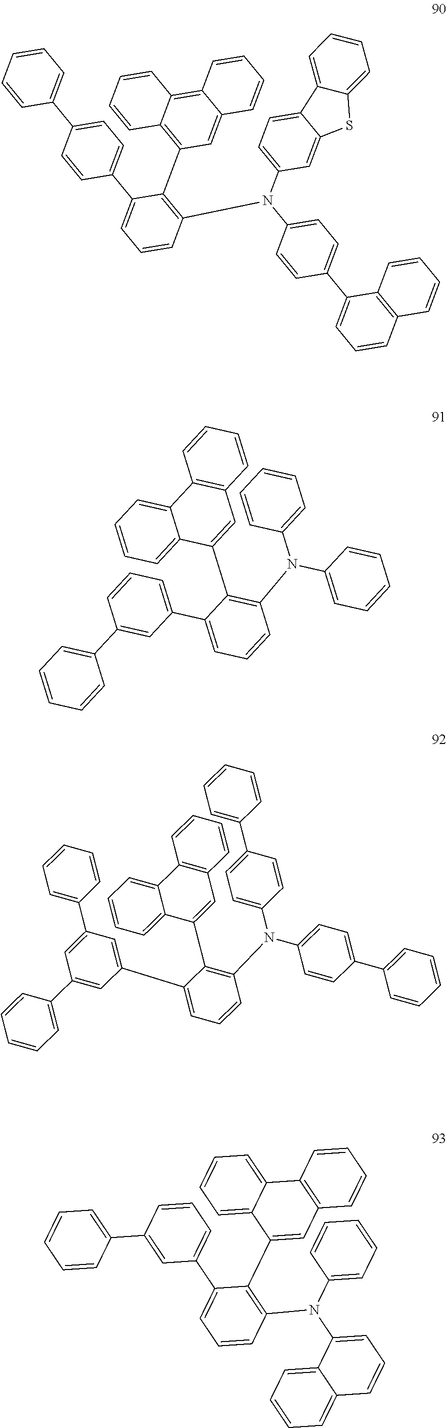 [SMILES: c1ccc(-c2ccc(-c3cccc(N(c4ccc(-c5cccc6ccccc56)cc4)c4ccc5c(c4)sc4ccccc45)c3-c3cc4ccccc4c4ccccc34)cc2)cc1 ; c1ccc(-c2cccc(-c3cccc(N(c4ccccc4)c4ccccc4)c3-c3cc4ccccc4c4ccccc34)c2)cc1 ; c1ccc(-c2ccc(N(c3ccc(-c4ccccc4)cc3)c3cccc(-c4cc(-c5ccccc5)cc(-c5ccccc5)c4)c3-c3cc4ccccc4c4ccccc34)cc2)cc1 ; c1ccc(-c2cccc(-c3cccc(N(c4ccccc4)c4cccc5ccccc45)c3-c3cc4ccccc4c4ccccc34)c2)cc1]

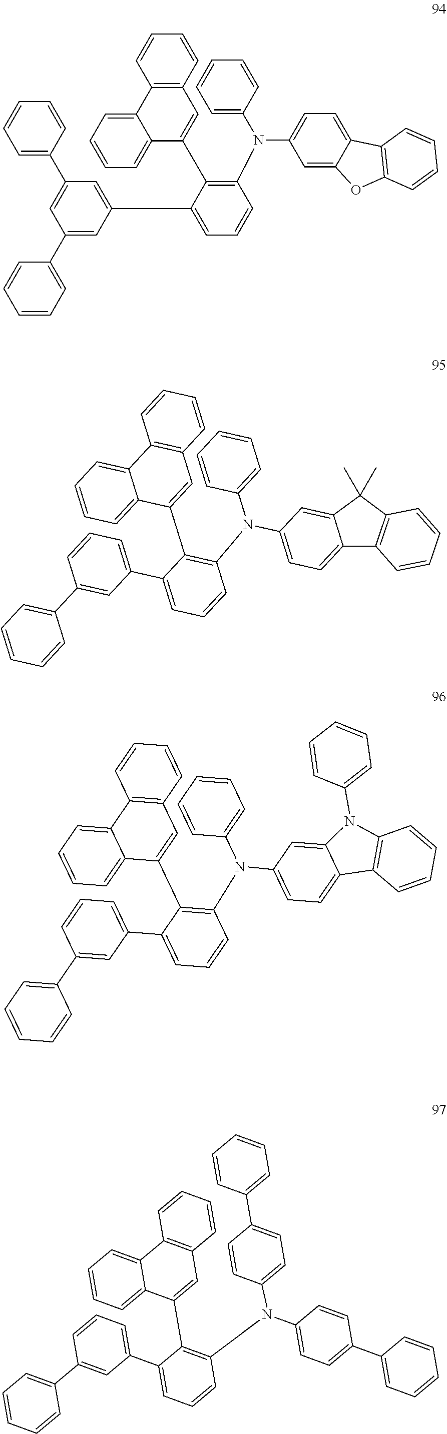 [SMILES: c1ccc(-c2cc(-c3ccccc3)cc(-c3cccc(N(c4ccccc4)c4ccc5c(c4)oc4ccccc45)c3-c3cc4ccccc4c4ccccc34)c2)cc1 ; CC1(C)c2ccccc2-c2ccc(N(c3ccccc3)c3cccc(-c4cccc(-c5ccccc5)c4)c3-c3cc4ccccc4c4ccccc34)cc21 ; c1ccc(-c2cccc(-c3cccc(N(c4ccccc4)c4ccc5c6ccccc6n(-c6ccccc6)c5c4)c3-c3cc4ccccc4c4ccccc34)c2)cc1 ; c1ccc(-c2ccc(N(c3ccc(-c4ccccc4)cc3)c3cccc(-c4cccc(-c5ccccc5)c4)c3-c3cc4ccccc4c4ccccc34)cc2)cc1]

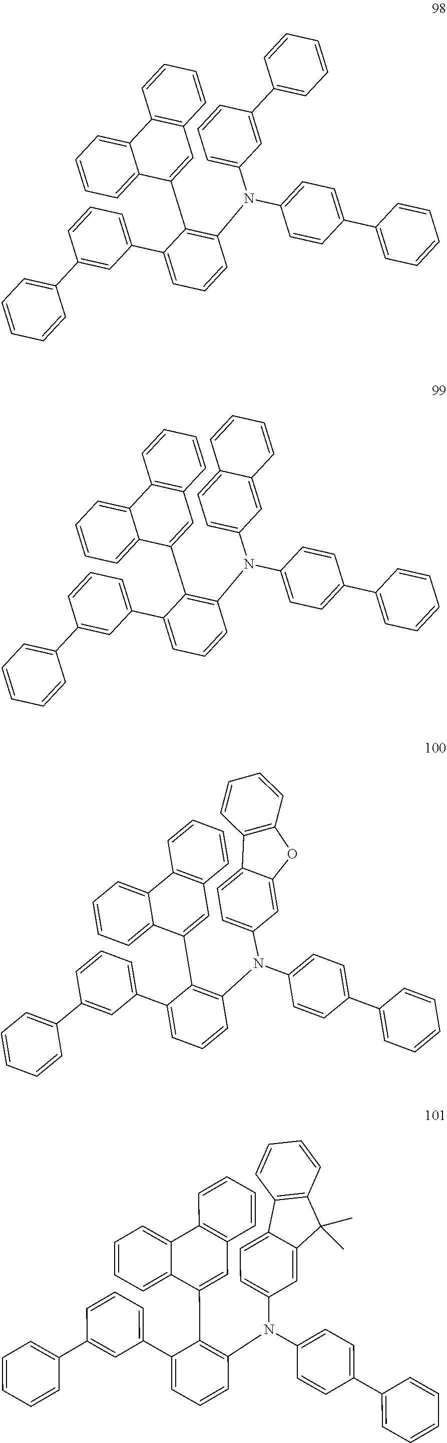 [SMILES: c1ccc(-c2ccc(N(c3cccc(-c4ccccc4)c3)c3cccc(-c4cccc(-c5ccccc5)c4)c3-c3cc4ccccc4c4ccccc34)cc2)cc1 ; c1ccc(-c2ccc(N(c3ccc4ccccc4c3)c3cccc(-c4cccc(-c5ccccc5)c4)c3-c3cc4ccccc4c4ccccc34)cc2)cc1 ; c1ccc(-c2ccc(N(c3ccc4c(c3)oc3ccccc34)c3cccc(-c4cccc(-c5ccccc5)c4)c3-c3cc4ccccc4c4ccccc34)cc2)cc1 ; CC1(C)c2ccccc2-c2ccc(N(c3ccc(-c4ccccc4)cc3)c3cccc(-c4cccc(-c5ccccc5)c4)c3-c3cc4ccccc4c4ccccc34)cc21]

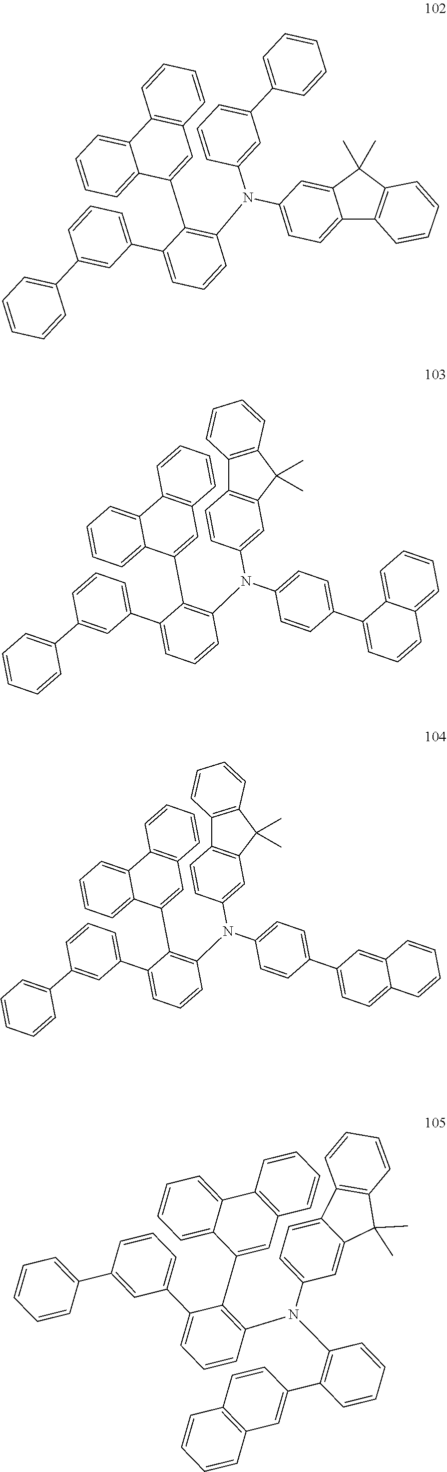 [SMILES: CC1(C)c2ccccc2-c2ccc(N(c3cccc(-c4ccccc4)c3)c3cccc(-c4cccc(-c5ccccc5)c4)c3-c3cc4ccccc4c4ccccc34)cc21 ; CC1(C)c2ccccc2-c2ccc(N(c3ccc(-c4cccc5ccccc45)cc3)c3cccc(-c4cccc(-c5ccccc5)c4)c3-c3cc4ccccc4c4ccccc34)cc21 ; CC1(C)c2ccccc2-c2ccc(N(c3ccc(-c4ccc5ccccc5c4)cc3)c3cccc(-c4cccc(-c5ccccc5)c4)c3-c3cc4ccccc4c4ccccc34)cc21 ; CC1(C)c2ccccc2-c2ccc(N(c3ccccc3-c3ccc4ccccc4c3)c3cccc(-c4cccc(-c5ccccc5)c4)c3-c3cc4ccccc4c4ccccc34)cc21]

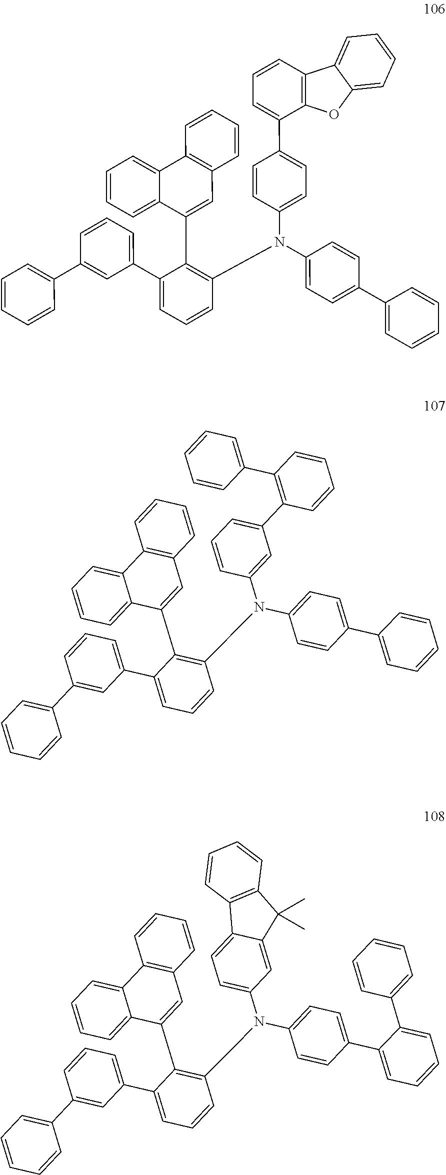 [SMILES: c1ccc(-c2ccc(N(c3ccc(-c4cccc5c4oc4ccccc45)cc3)c3cccc(-c4cccc(-c5ccccc5)c4)c3-c3cc4ccccc4c4ccccc34)cc2)cc1 ; c1ccc(-c2ccc(N(c3cccc(-c4ccccc4-c4ccccc4)c3)c3cccc(-c4cccc(-c5ccccc5)c4)c3-c3cc4ccccc4c4ccccc34)cc2)cc1 ; CC1(C)c2ccccc2-c2ccc(N(c3ccc(-c4ccccc4-c4ccccc4)cc3)c3cccc(-c4cccc(-c5ccccc5)c4)c3-c3cc4ccccc4c4ccccc34)cc21]

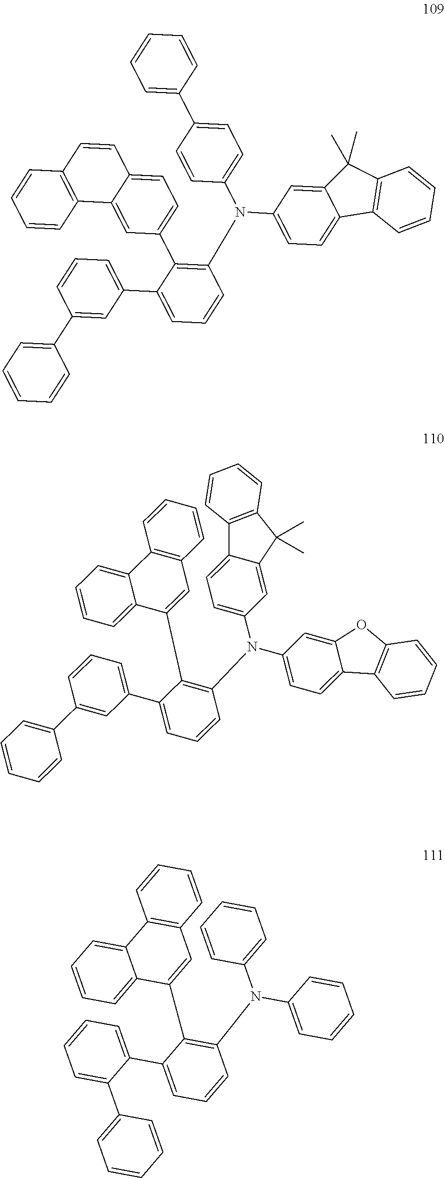 [SMILES: CC1(C)c2ccccc2-c2ccc(N(c3ccc(-c4ccccc4)cc3)c3cccc(-c4cccc(-c5ccccc5)c4)c3-c3ccc4ccc5ccccc5c4c3)cc21 ; CC1(C)c2ccccc2-c2ccc(N(c3ccc4c(c3)oc3ccccc34)c3cccc(-c4cccc(-c5ccccc5)c4)c3-c3cc4ccccc4c4ccccc34)cc21 ; c1ccc(-c2ccccc2-c2cccc(N(c3ccccc3)c3ccccc3)c2-c2cc3ccccc3c3ccccc23)cc1]

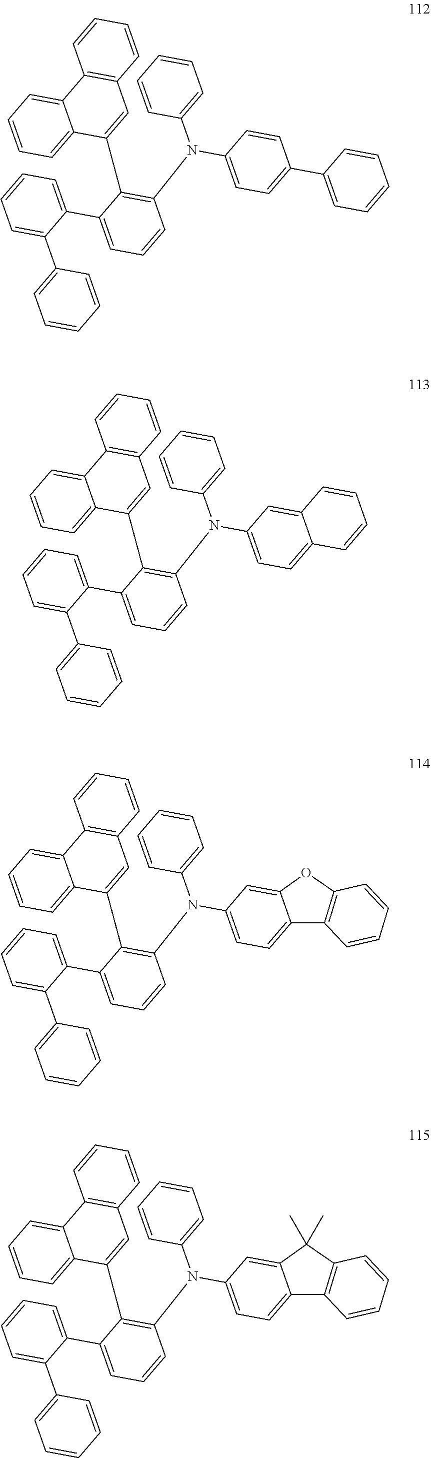 [SMILES: c1ccc(-c2ccc(N(c3ccccc3)c3cccc(-c4ccccc4-c4ccccc4)c3-c3cc4ccccc4c4ccccc34)cc2)cc1 ; c1ccc(-c2ccccc2-c2cccc(N(c3ccccc3)c3ccc4ccccc4c3)c2-c2cc3ccccc3c3ccccc23)cc1 ; c1ccc(-c2ccccc2-c2cccc(N(c3ccccc3)c3ccc4c(c3)oc3ccccc34)c2-c2cc3ccccc3c3ccccc23)cc1 ; CC1(C)c2ccccc2-c2ccc(N(c3ccccc3)c3cccc(-c4ccccc4-c4ccccc4)c3-c3cc4ccccc4c4ccccc34)cc21]

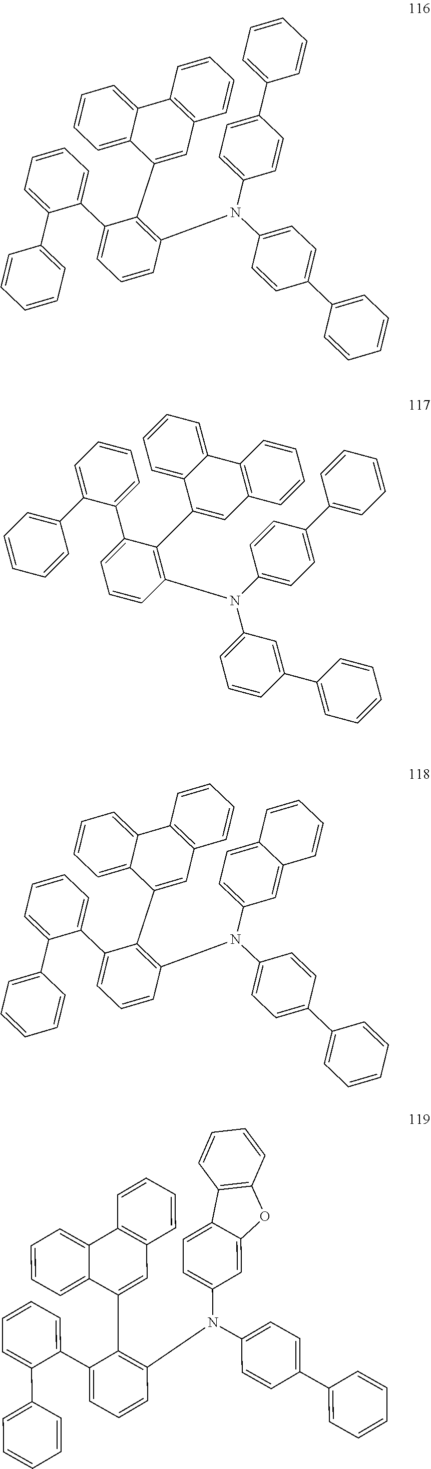 [SMILES: c1ccc(-c2ccc(N(c3ccc(-c4ccccc4)cc3)c3cccc(-c4ccccc4-c4ccccc4)c3-c3cc4ccccc4c4ccccc34)cc2)cc1 ; c1ccc(-c2ccc(N(c3cccc(-c4ccccc4)c3)c3cccc(-c4ccccc4-c4ccccc4)c3-c3cc4ccccc4c4ccccc34)cc2)cc1 ; c1ccc(-c2ccc(N(c3ccc4ccccc4c3)c3cccc(-c4ccccc4-c4ccccc4)c3-c3cc4ccccc4c4ccccc34)cc2)cc1 ; c1ccc(-c2ccc(N(c3ccc4c(c3)oc3ccccc34)c3cccc(-c4ccccc4-c4ccccc4)c3-c3cc4ccccc4c4ccccc34)cc2)cc1]

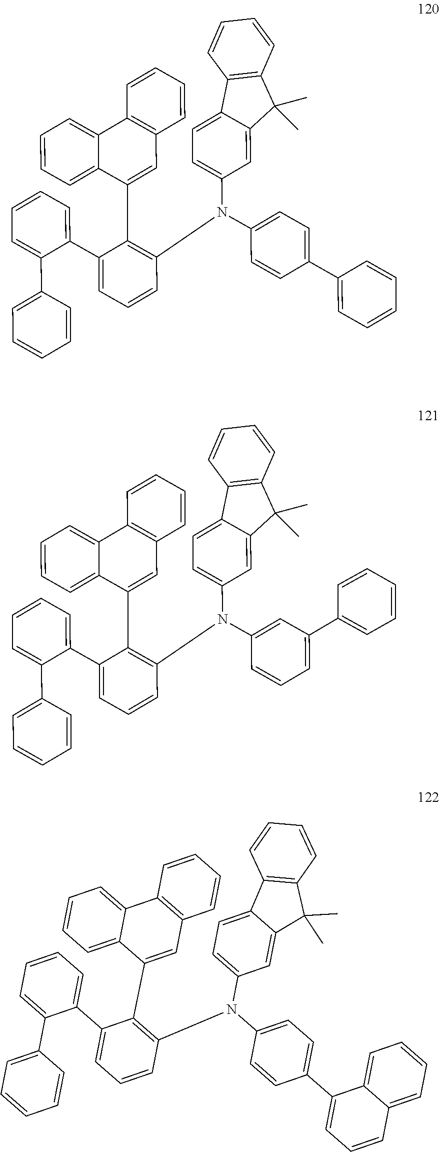 [SMILES: CC1(C)c2ccccc2-c2ccc(N(c3ccc(-c4ccccc4)cc3)c3cccc(-c4ccccc4-c4ccccc4)c3-c3cc4ccccc4c4ccccc34)cc21 ; CC1(C)c2ccccc2-c2ccc(N(c3cccc(-c4ccccc4)c3)c3cccc(-c4ccccc4-c4ccccc4)c3-c3cc4ccccc4c4ccccc34)cc21 ; CC1(C)c2ccccc2-c2ccc(N(c3ccc(-c4cccc5ccccc45)cc3)c3cccc(-c4ccccc4-c4ccccc4)c3-c3cc4ccccc4c4ccccc34)cc21]

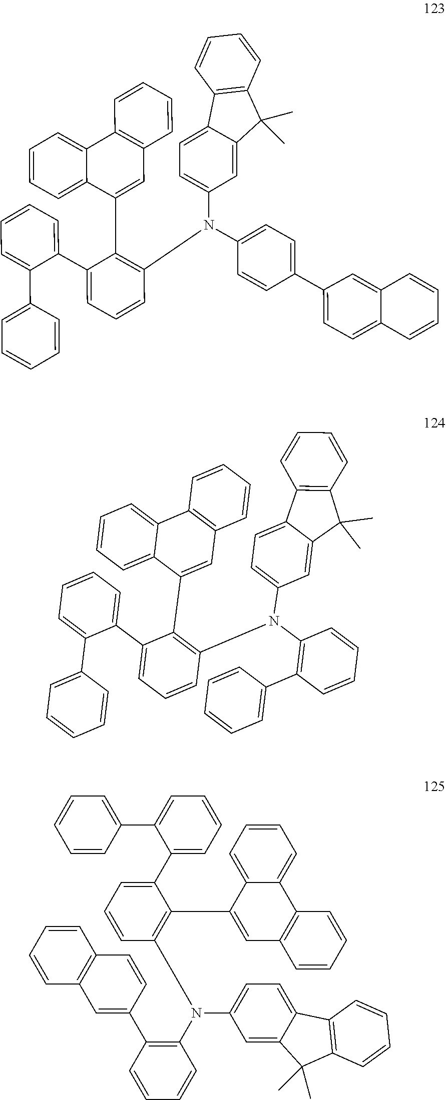 [SMILES: CC1(C)c2ccccc2-c2ccc(N(c3ccc(-c4ccc5ccccc5c4)cc3)c3cccc(-c4ccccc4-c4ccccc4)c3-c3cc4ccccc4c4ccccc34)cc21 ; CC1(C)c2ccccc2-c2ccc(N(c3ccccc3-c3ccccc3)c3cccc(-c4ccccc4-c4ccccc4)c3-c3cc4ccccc4c4ccccc34)cc21 ; CC1(C)c2ccccc2-c2ccc(N(c3ccccc3-c3ccc4ccccc4c3)c3cccc(-c4ccccc4-c4ccccc4)c3-c3cc4ccccc4c4ccccc34)cc21]

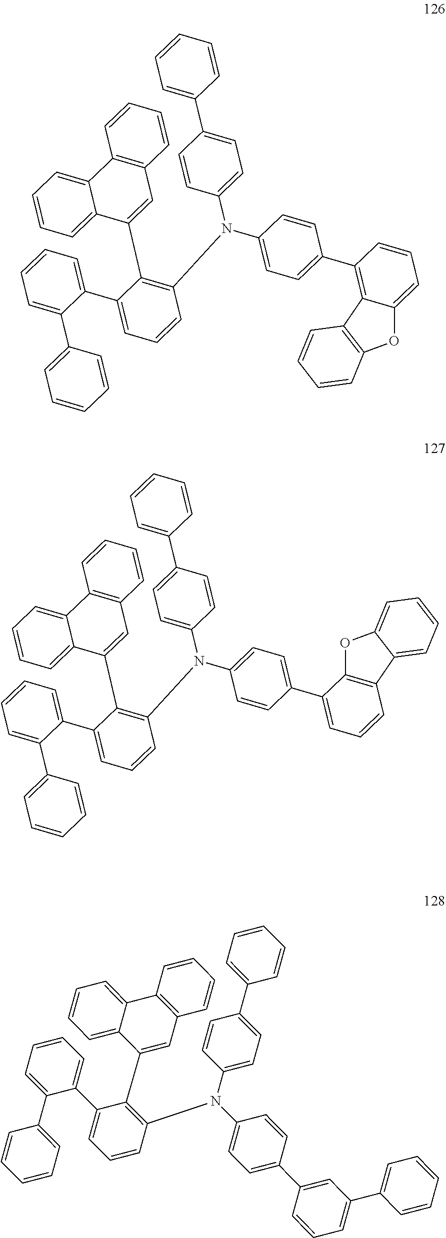 [SMILES: c1ccc(-c2ccc(N(c3ccc(-c4cccc5oc6ccccc6c45)cc3)c3cccc(-c4ccccc4-c4ccccc4)c3-c3cc4ccccc4c4ccccc34)cc2)cc1 ; c1ccc(-c2ccc(N(c3ccc(-c4cccc5c4oc4ccccc45)cc3)c3cccc(-c4ccccc4-c4ccccc4)c3-c3cc4ccccc4c4ccccc34)cc2)cc1 ; c1ccc(-c2ccc(N(c3ccc(-c4cccc(-c5ccccc5)c4)cc3)c3cccc(-c4ccccc4-c4ccccc4)c3-c3cc4ccccc4c4ccccc34)cc2)cc1]

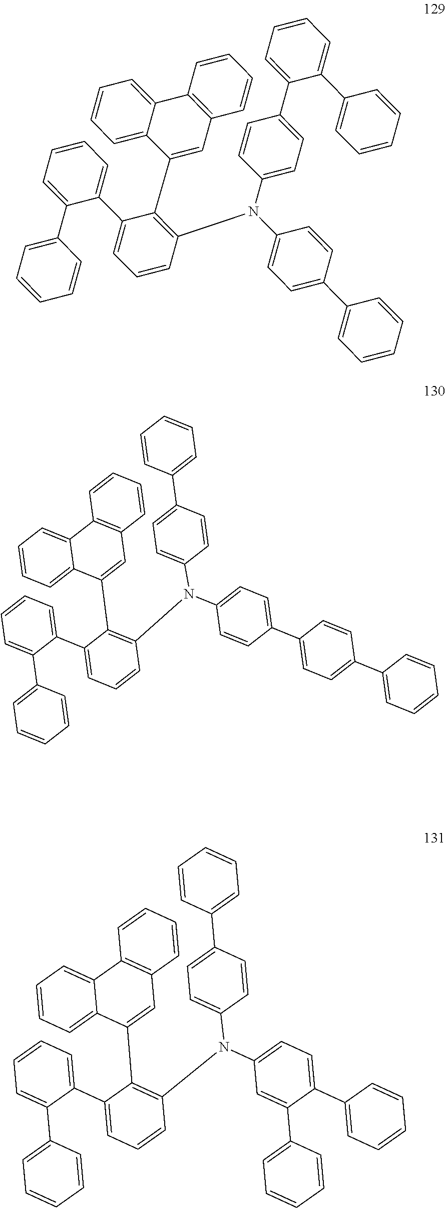 [SMILES: c1ccc(-c2ccc(N(c3ccc(-c4ccccc4-c4ccccc4)cc3)c3cccc(-c4ccccc4-c4ccccc4)c3-c3cc4ccccc4c4ccccc34)cc2)cc1 ; c1ccc(-c2ccc(-c3ccc(N(c4ccc(-c5ccccc5)cc4)c4cccc(-c5ccccc5-c5ccccc5)c4-c4cc5ccccc5c5ccccc45)cc3)cc2)cc1 ; c1ccc(-c2ccc(N(c3ccc(-c4ccccc4)c(-c4ccccc4)c3)c3cccc(-c4ccccc4-c4ccccc4)c3-c3cc4ccccc4c4ccccc34)cc2)cc1]

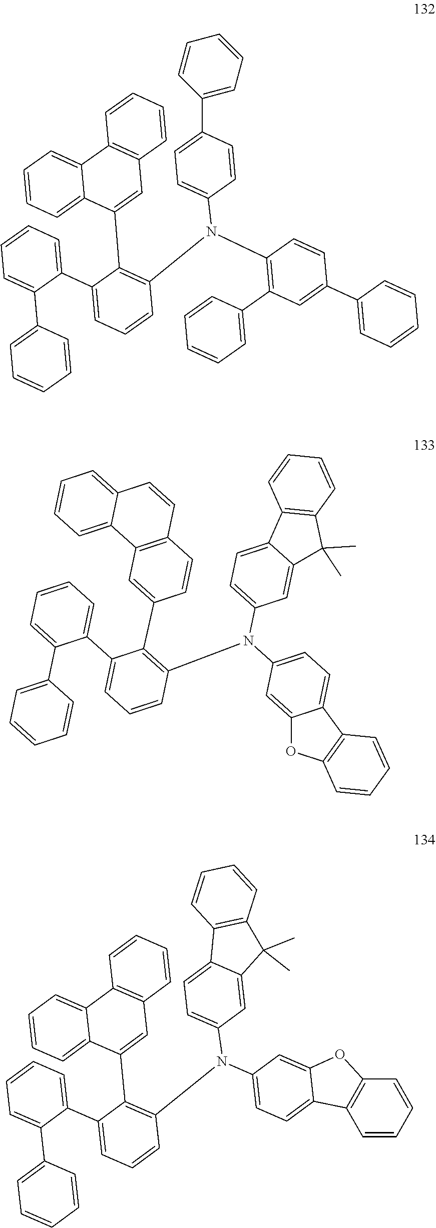 [SMILES: c1ccc(-c2ccc(N(c3ccc(-c4ccccc4)cc3-c3ccccc3)c3cccc(-c4ccccc4-c4ccccc4)c3-c3cc4ccccc4c4ccccc34)cc2)cc1 ; CC1(C)c2ccccc2-c2ccc(N(c3ccc4c(c3)oc3ccccc34)c3cccc(-c4ccccc4-c4ccccc4)c3-c3ccc4ccc5ccccc5c4c3)cc21 ; CC1(C)c2ccccc2-c2ccc(N(c3ccc4c(c3)oc3ccccc34)c3cccc(-c4ccccc4-c4ccccc4)c3-c3cc4ccccc4c4ccccc34)cc21]

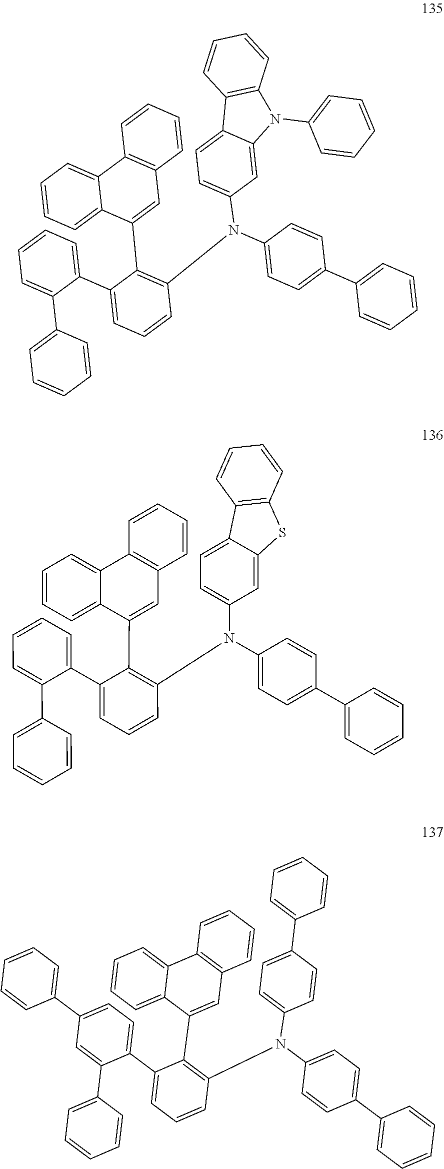 [SMILES: c1ccc(-c2ccc(N(c3ccc4c5ccccc5n(-c5ccccc5)c4c3)c3cccc(-c4ccccc4-c4ccccc4)c3-c3cc4ccccc4c4ccccc34)cc2)cc1 ; c1ccc(-c2ccc(N(c3ccc4c(c3)sc3ccccc34)c3cccc(-c4ccccc4-c4ccccc4)c3-c3cc4ccccc4c4ccccc34)cc2)cc1 ; c1ccc(-c2ccc(N(c3ccc(-c4ccccc4)cc3)c3cccc(-c4ccc(-c5ccccc5)cc4-c4ccccc4)c3-c3cc4ccccc4c4ccccc34)cc2)cc1]

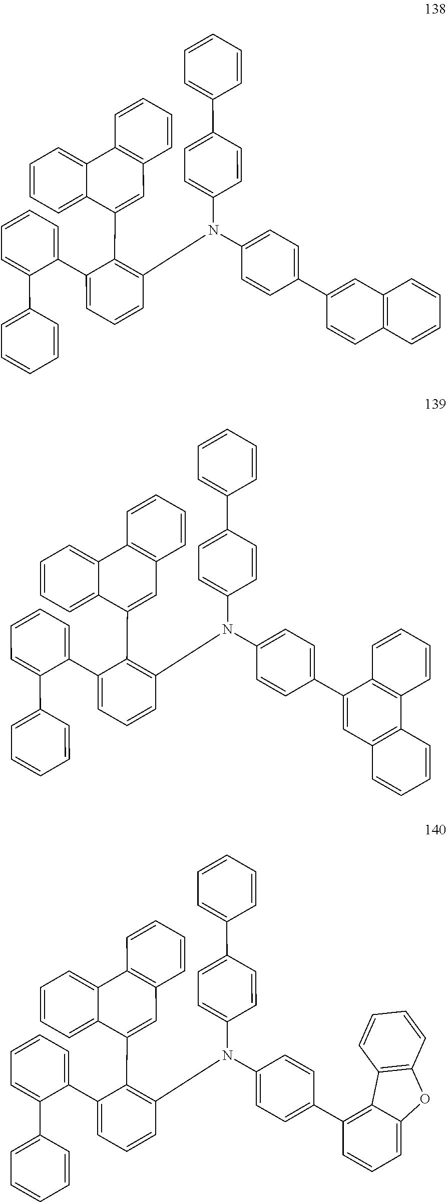 [SMILES: c1ccc(-c2ccc(N(c3ccc(-c4ccc5ccccc5c4)cc3)c3cccc(-c4ccccc4-c4ccccc4)c3-c3cc4ccccc4c4ccccc34)cc2)cc1 ; c1ccc(-c2ccc(N(c3ccc(-c4cc5ccccc5c5ccccc45)cc3)c3cccc(-c4ccccc4-c4ccccc4)c3-c3cc4ccccc4c4ccccc34)cc2)cc1 ; c1ccc(-c2ccc(N(c3ccc(-c4cccc5oc6ccccc6c45)cc3)c3cccc(-c4ccccc4-c4ccccc4)c3-c3cc4ccccc4c4ccccc34)cc2)cc1]

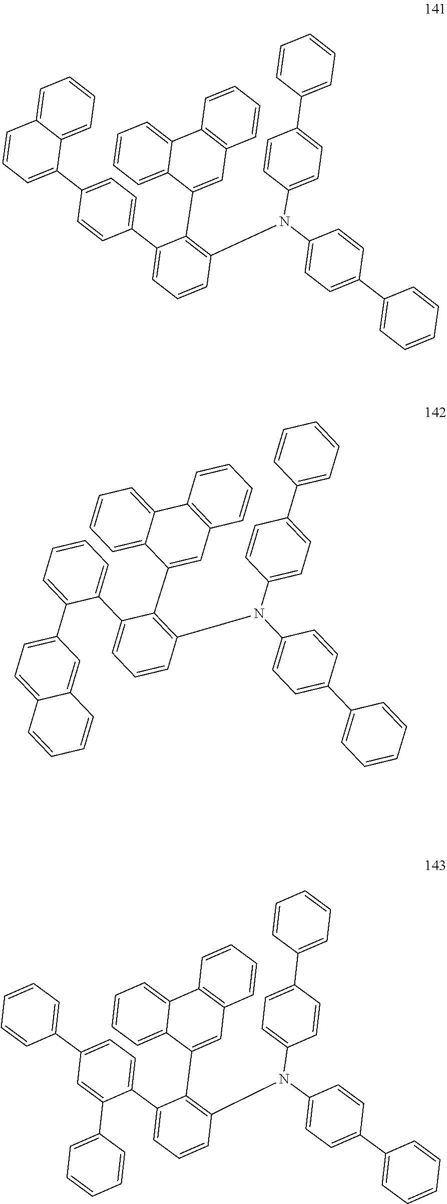 [SMILES: c1ccc(-c2ccc(N(c3ccc(-c4ccccc4)cc3)c3cccc(-c4ccc(-c5cccc6ccccc56)cc4)c3-c3cc4ccccc4c4ccccc34)cc2)cc1 ; c1ccc(-c2ccc(N(c3ccc(-c4ccccc4)cc3)c3cccc(-c4ccccc4-c4ccc5ccccc5c4)c3-c3cc4ccccc4c4ccccc34)cc2)cc1 ; c1ccc(-c2ccc(N(c3ccc(-c4ccccc4)cc3)c3cccc(-c4ccc(-c5ccccc5)cc4-c4ccccc4)c3-c3cc4ccccc4c4ccccc34)cc2)cc1]

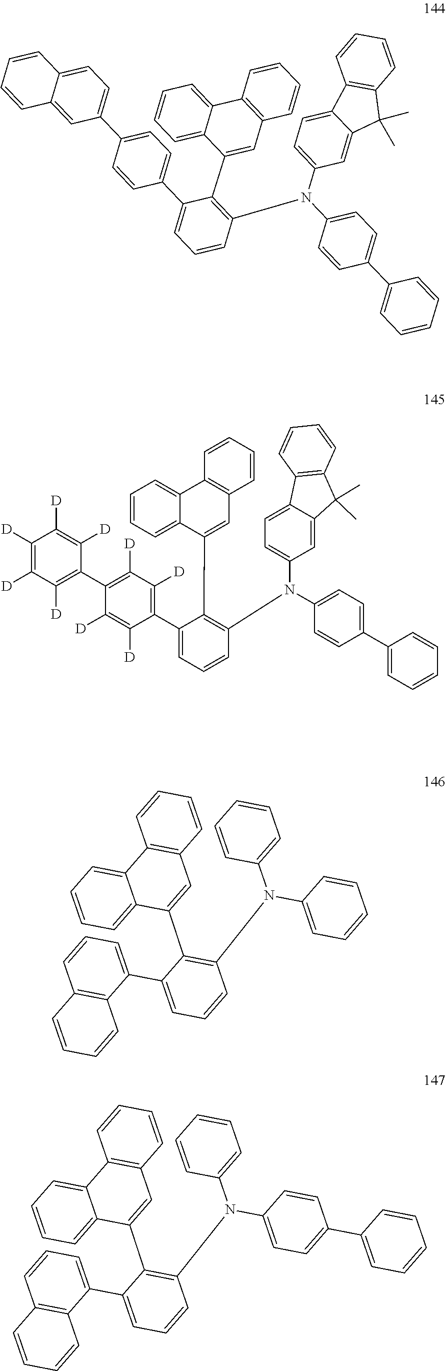 [SMILES: CC1(C)c2ccccc2-c2ccc(N(c3ccc(-c4ccccc4)cc3)c3cccc(-c4ccc(-c5ccc6ccccc6c5)cc4)c3-c3cc4ccccc4c4ccccc34)cc21 ; [2H]c1c([2H])c([2H])c(-c2c([2H])c([2H])c(-c3cccc(N(c4ccc(-c5ccccc5)cc4)c4ccc5c(c4)C(C)(C)c4ccccc4-5)c3-c3cc4ccccc4c4ccccc34)c([2H])c2[2H])c([2H])c1[2H] ; c1ccc(N(c2ccccc2)c2cccc(-c3cccc4ccccc34)c2-c2cc3ccccc3c3ccccc23)cc1 ; c1ccc(-c2ccc(N(c3ccccc3)c3cccc(-c4cccc5ccccc45)c3-c3cc4ccccc4c4ccccc34)cc2)cc1]

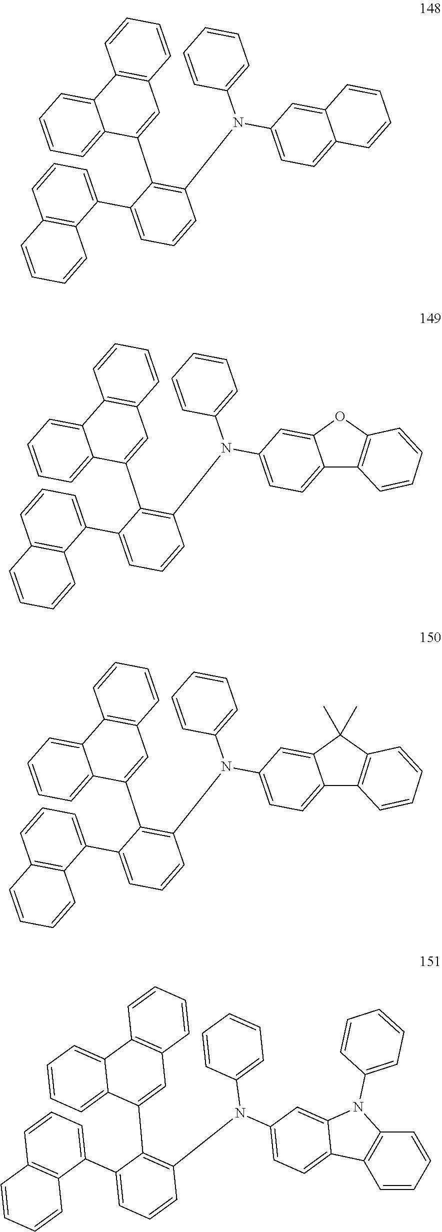 [SMILES: c1ccc(N(c2ccc3ccccc3c2)c2cccc(-c3cccc4ccccc34)c2-c2cc3ccccc3c3ccccc23)cc1 ; c1ccc(N(c2ccc3c(c2)oc2ccccc23)c2cccc(-c3cccc4ccccc34)c2-c2cc3ccccc3c3ccccc23)cc1 ; CC1(C)c2ccccc2-c2ccc(N(c3ccccc3)c3cccc(-c4cccc5ccccc45)c3-c3cc4ccccc4c4ccccc34)cc21 ; c1ccc(N(c2ccc3c4ccccc4n(-c4ccccc4)c3c2)c2cccc(-c3cccc4ccccc34)c2-c2cc3ccccc3c3ccccc23)cc1]

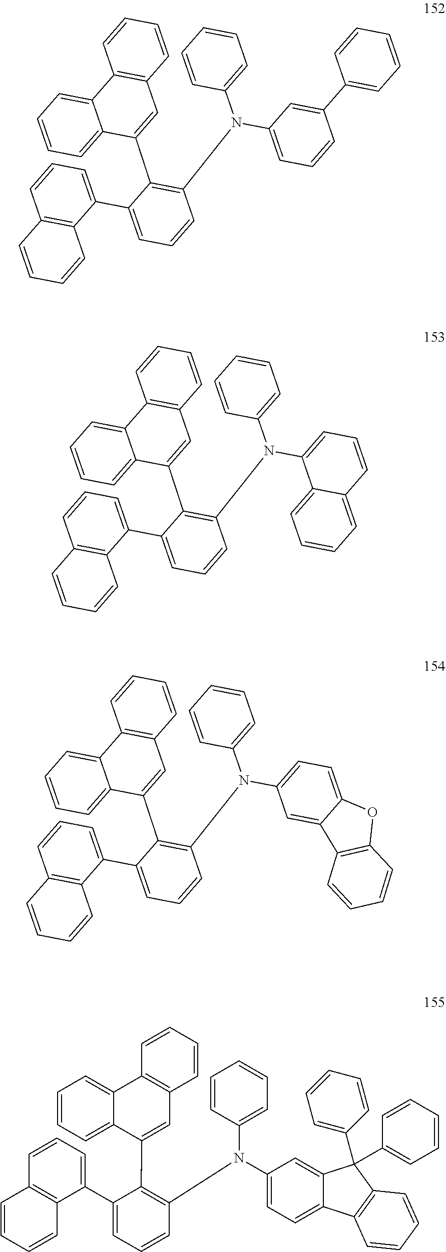 [SMILES: c1ccc(-c2cccc(N(c3ccccc3)c3cccc(-c4cccc5ccccc45)c3-c3cc4ccccc4c4ccccc34)c2)cc1 ; c1ccc(N(c2cccc(-c3cccc4ccccc34)c2-c2cc3ccccc3c3ccccc23)c2cccc3ccccc23)cc1 ; c1ccc(N(c2ccc3oc4ccccc4c3c2)c2cccc(-c3cccc4ccccc34)c2-c2cc3ccccc3c3ccccc23)cc1 ; c1ccc(N(c2ccc3c(c2)C(c2ccccc2)(c2ccccc2)c2ccccc2-3)c2cccc(-c3cccc4ccccc34)c2-c2cc3ccccc3c3ccccc23)cc1]

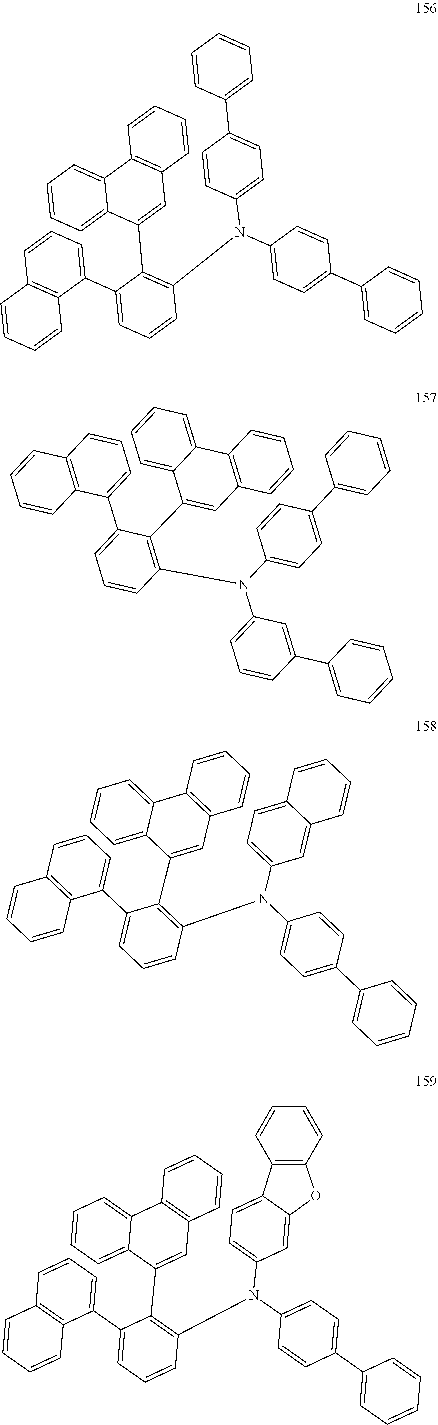 [SMILES: c1ccc(-c2ccc(N(c3ccc(-c4ccccc4)cc3)c3cccc(-c4cccc5ccccc45)c3-c3cc4ccccc4c4ccccc34)cc2)cc1 ; c1ccc(-c2ccc(N(c3cccc(-c4ccccc4)c3)c3cccc(-c4cccc5ccccc45)c3-c3cc4ccccc4c4ccccc34)cc2)cc1 ; c1ccc(-c2ccc(N(c3ccc4ccccc4c3)c3cccc(-c4cccc5ccccc45)c3-c3cc4ccccc4c4ccccc34)cc2)cc1 ; c1ccc(-c2ccc(N(c3ccc4c(c3)oc3ccccc34)c3cccc(-c4cccc5ccccc45)c3-c3cc4ccccc4c4ccccc34)cc2)cc1]

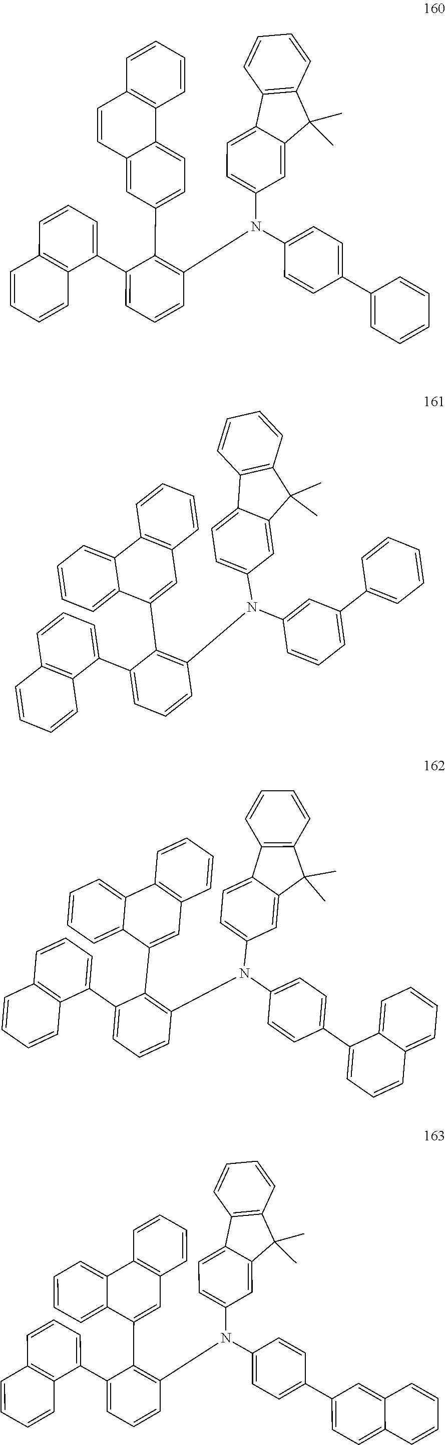 [SMILES: CC1(C)c2ccccc2-c2ccc(N(c3ccc(-c4ccccc4)cc3)c3cccc(-c4cccc5ccccc45)c3-c3ccc4c(ccc5ccccc54)c3)cc21 ; CC1(C)c2ccccc2-c2ccc(N(c3cccc(-c4ccccc4)c3)c3cccc(-c4cccc5ccccc45)c3-c3cc4ccccc4c4ccccc34)cc21 ; CC1(C)c2ccccc2-c2ccc(N(c3ccc(-c4cccc5ccccc45)cc3)c3cccc(-c4cccc5ccccc45)c3-c3cc4ccccc4c4ccccc34)cc21 ; CC1(C)c2ccccc2-c2ccc(N(c3ccc(-c4ccc5ccccc5c4)cc3)c3cccc(-c4cccc5ccccc45)c3-c3cc4ccccc4c4ccccc34)cc21]

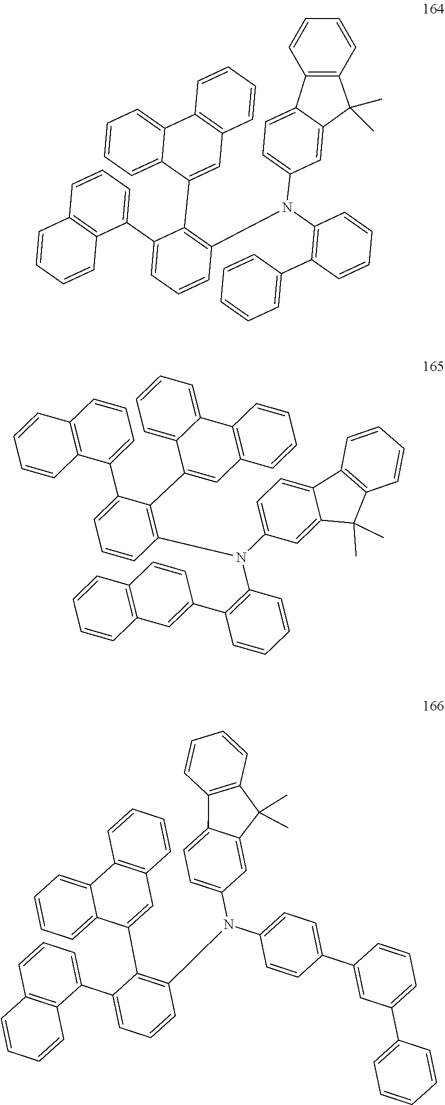 [SMILES: CC1(C)c2ccccc2-c2ccc(N(c3ccccc3-c3ccccc3)c3cccc(-c4cccc5ccccc45)c3-c3cc4ccccc4c4ccccc34)cc21 ; CC1(C)c2ccccc2-c2ccc(N(c3ccccc3-c3ccc4ccccc4c3)c3cccc(-c4cccc5ccccc45)c3-c3cc4ccccc4c4ccccc34)cc21 ; CC1(C)c2ccccc2-c2ccc(N(c3ccc(-c4cccc(-c5ccccc5)c4)cc3)c3cccc(-c4cccc5ccccc45)c3-c3cc4ccccc4c4ccccc34)cc21]

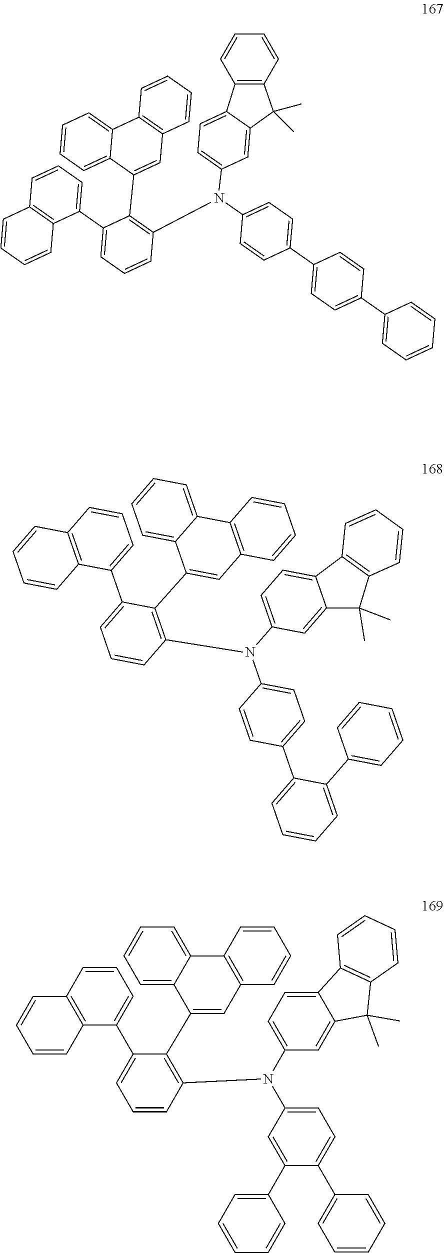 [SMILES: CC1(C)c2ccccc2-c2ccc(N(c3ccc(-c4ccc(-c5ccccc5)cc4)cc3)c3cccc(-c4cccc5ccccc45)c3-c3cc4ccccc4c4ccccc34)cc21 ; CC1(C)c2ccccc2-c2ccc(N(c3ccc(-c4ccccc4-c4ccccc4)cc3)c3cccc(-c4cccc5ccccc45)c3-c3cc4ccccc4c4ccccc34)cc21 ; CC1(C)c2ccccc2-c2ccc(N(c3ccc(-c4ccccc4)c(-c4ccccc4)c3)c3cccc(-c4cccc5ccccc45)c3-c3cc4ccccc4c4ccccc34)cc21]

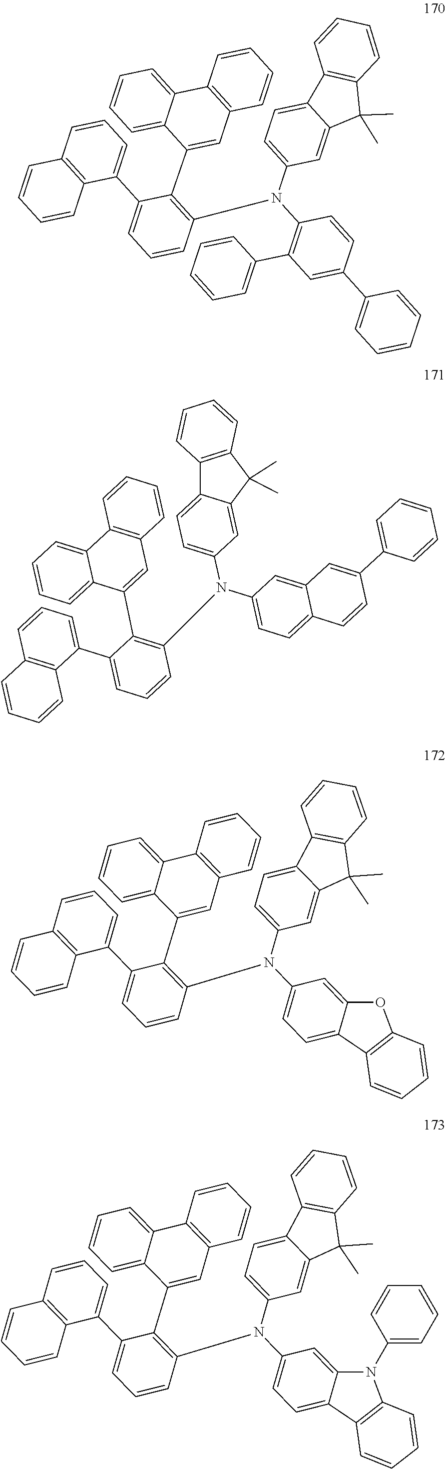 [SMILES: CC1(C)c2ccccc2-c2ccc(N(c3ccc(-c4ccccc4)cc3-c3ccccc3)c3cccc(-c4cccc5ccccc45)c3-c3cc4ccccc4c4ccccc34)cc21 ; CC1(C)c2ccccc2-c2ccc(N(c3ccc4ccc(-c5ccccc5)cc4c3)c3cccc(-c4cccc5ccccc45)c3-c3cc4ccccc4c4ccccc34)cc21 ; CC1(C)c2ccccc2-c2ccc(N(c3ccc4c(c3)oc3ccccc34)c3cccc(-c4cccc5ccccc45)c3-c3cc4ccccc4c4ccccc34)cc21 ; CC1(C)c2ccccc2-c2ccc(N(c3ccc4c5ccccc5n(-c5ccccc5)c4c3)c3cccc(-c4cccc5ccccc45)c3-c3cc4ccccc4c4ccccc34)cc21]

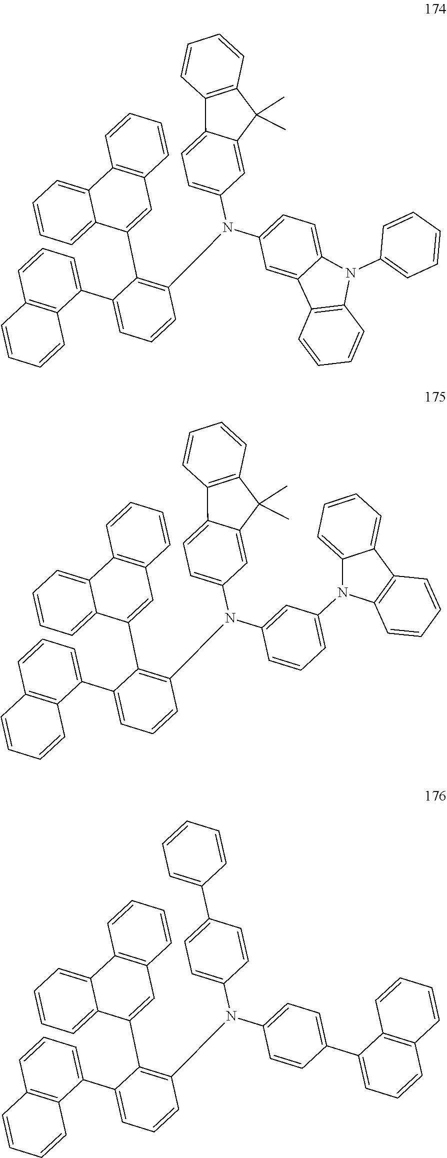 [SMILES: CC1(C)c2ccccc2-c2ccc(N(c3ccc4c(c3)c3ccccc3n4-c3ccccc3)c3cccc(-c4cccc5ccccc45)c3-c3cc4ccccc4c4ccccc34)cc21 ; CC1(C)c2ccccc2-c2ccc(N(c3cccc(-n4c5ccccc5c5ccccc54)c3)c3cccc(-c4cccc5ccccc45)c3-c3cc4ccccc4c4ccccc34)cc21 ; c1ccc(-c2ccc(N(c3ccc(-c4cccc5ccccc45)cc3)c3cccc(-c4cccc5ccccc45)c3-c3cc4ccccc4c4ccccc34)cc2)cc1]

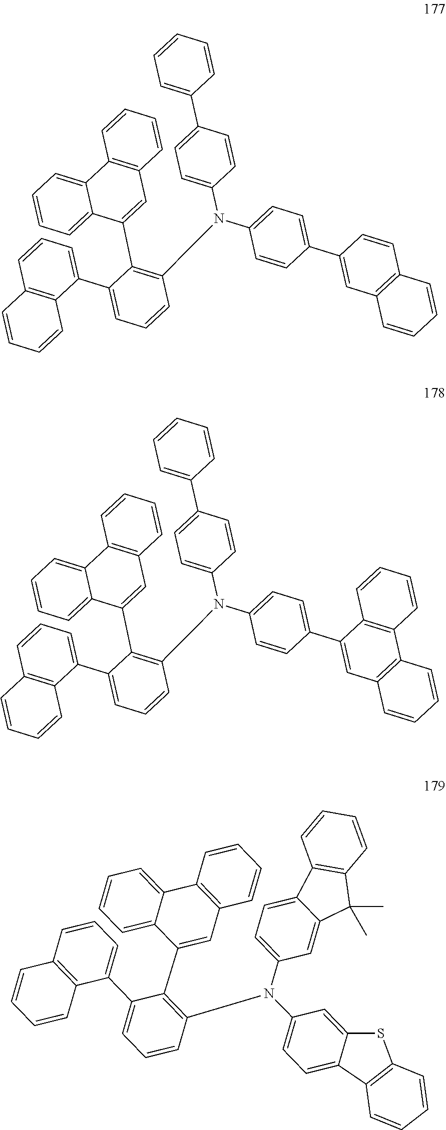 [SMILES: c1ccc(-c2ccc(N(c3ccc(-c4ccc5ccccc5c4)cc3)c3cccc(-c4cccc5ccccc45)c3-c3cc4ccccc4c4ccccc34)cc2)cc1 ; c1ccc(-c2ccc(N(c3ccc(-c4cc5ccccc5c5ccccc45)cc3)c3cccc(-c4cccc5ccccc45)c3-c3cc4ccccc4c4ccccc34)cc2)cc1 ; CC1(C)c2ccccc2-c2ccc(N(c3ccc4c(c3)sc3ccccc34)c3cccc(-c4cccc5ccccc45)c3-c3cc4ccccc4c4ccccc34)cc21]

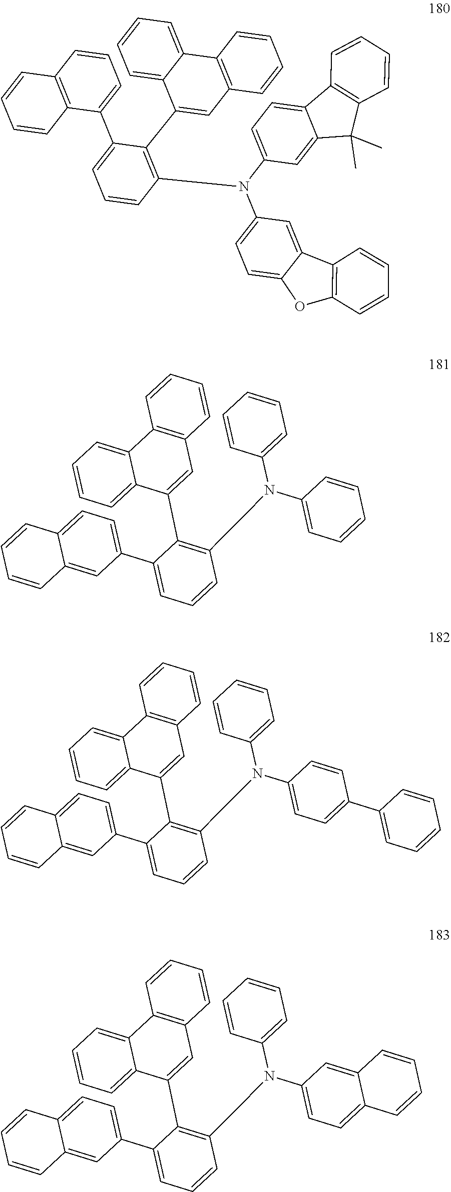 [SMILES: CC1(C)c2ccccc2-c2ccc(N(c3ccc4oc5ccccc5c4c3)c3cccc(-c4cccc5ccccc45)c3-c3cc4ccccc4c4ccccc34)cc21 ; c1ccc(N(c2ccccc2)c2cccc(-c3ccc4ccccc4c3)c2-c2cc3ccccc3c3ccccc23)cc1 ; c1ccc(-c2ccc(N(c3ccccc3)c3cccc(-c4ccc5ccccc5c4)c3-c3cc4ccccc4c4ccccc34)cc2)cc1 ; c1ccc(N(c2ccc3ccccc3c2)c2cccc(-c3ccc4ccccc4c3)c2-c2cc3ccccc3c3ccccc23)cc1]

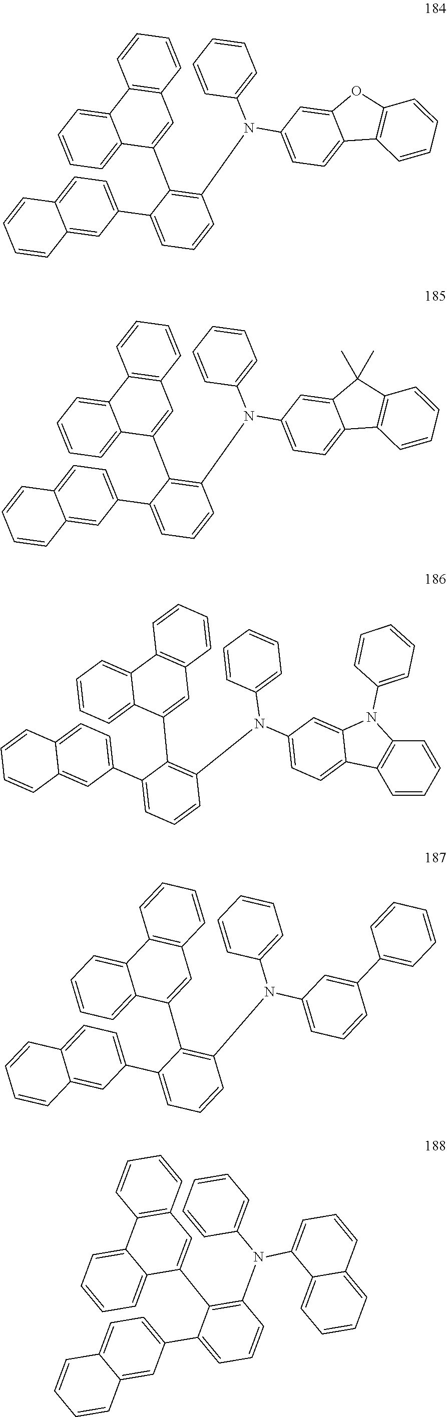 [SMILES: c1ccc(N(c2ccc3c(c2)oc2ccccc23)c2cccc(-c3ccc4ccccc4c3)c2-c2cc3ccccc3c3ccccc23)cc1 ; CC1(C)c2ccccc2-c2ccc(N(c3ccccc3)c3cccc(-c4ccc5ccccc5c4)c3-c3cc4ccccc4c4ccccc34)cc21 ; c1ccc(N(c2ccc3c4ccccc4n(-c4ccccc4)c3c2)c2cccc(-c3ccc4ccccc4c3)c2-c2cc3ccccc3c3ccccc23)cc1 ; c1ccc(-c2cccc(N(c3ccccc3)c3cccc(-c4ccc5ccccc5c4)c3-c3cc4ccccc4c4ccccc34)c2)cc1 ; c1ccc(N(c2cccc(-c3ccc4ccccc4c3)c2-c2cc3ccccc3c3ccccc23)c2cccc3ccccc23)cc1]

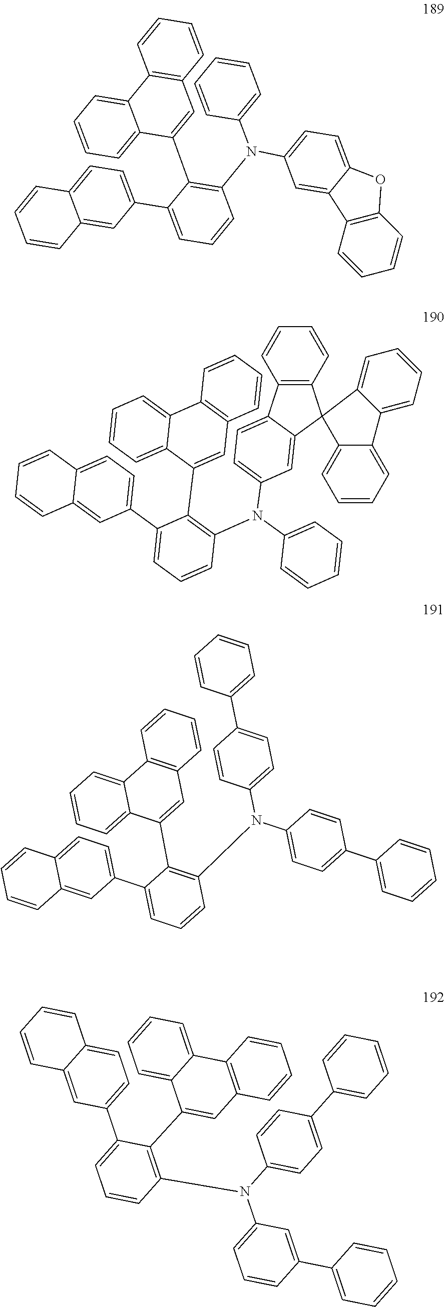 [SMILES: c1ccc(N(c2ccc3oc4ccccc4c3c2)c2cccc(-c3ccc4ccccc4c3)c2-c2cc3ccccc3c3ccccc23)cc1 ; c1ccc(N(c2ccc3c(c2)C2(c4ccccc4-c4ccccc42)c2ccccc2-3)c2cccc(-c3ccc4ccccc4c3)c2-c2cc3ccccc3c3ccccc23)cc1 ; c1ccc(-c2ccc(N(c3ccc(-c4ccccc4)cc3)c3cccc(-c4ccc5ccccc5c4)c3-c3cc4ccccc4c4ccccc34)cc2)cc1 ; c1ccc(-c2ccc(N(c3cccc(-c4ccccc4)c3)c3cccc(-c4ccc5ccccc5c4)c3-c3cc4ccccc4c4ccccc34)cc2)cc1]

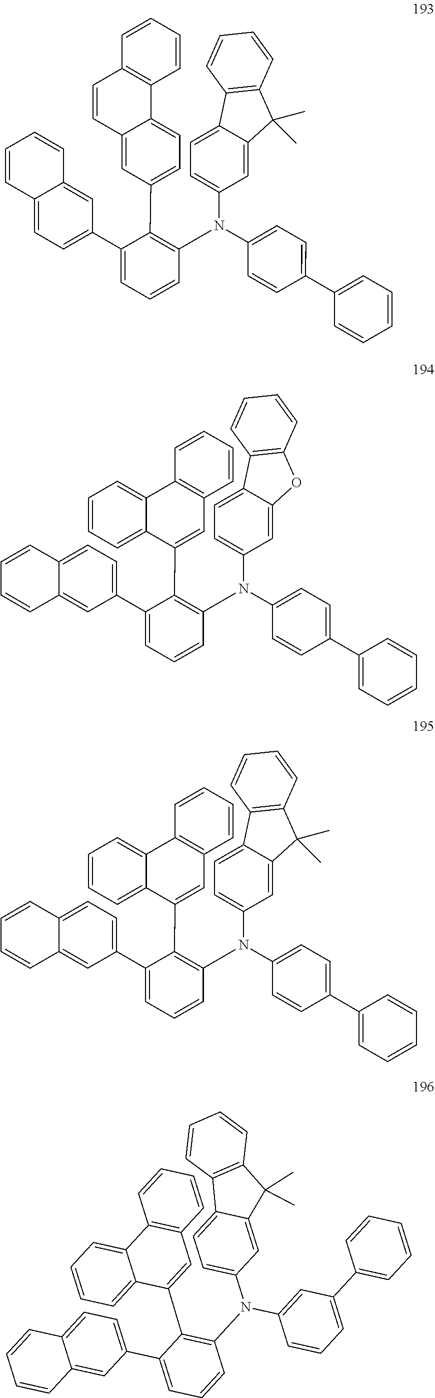 [SMILES: CC1(C)c2ccccc2-c2ccc(N(c3ccc(-c4ccccc4)cc3)c3cccc(-c4ccc5ccccc5c4)c3-c3ccc4c(ccc5ccccc54)c3)cc21 ; c1ccc(-c2ccc(N(c3ccc4c(c3)oc3ccccc34)c3cccc(-c4ccc5ccccc5c4)c3-c3cc4ccccc4c4ccccc34)cc2)cc1 ; CC1(C)c2ccccc2-c2ccc(N(c3ccc(-c4ccccc4)cc3)c3cccc(-c4ccc5ccccc5c4)c3-c3cc4ccccc4c4ccccc34)cc21 ; CC1(C)c2ccccc2-c2ccc(N(c3cccc(-c4ccccc4)c3)c3cccc(-c4ccc5ccccc5c4)c3-c3cc4ccccc4c4ccccc34)cc21]

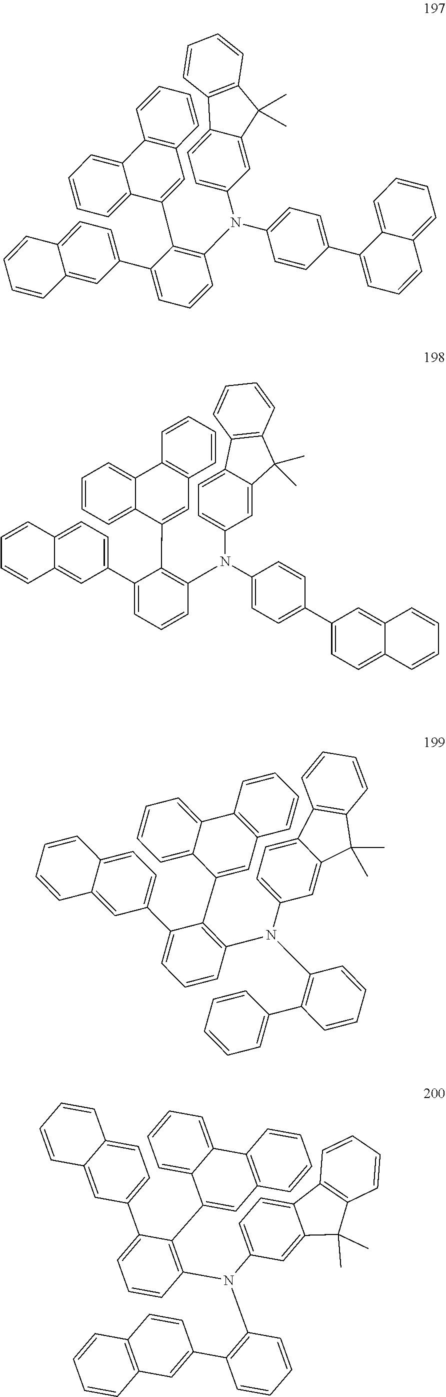 [SMILES: CC1(C)c2ccccc2-c2ccc(N(c3ccc(-c4cccc5ccccc45)cc3)c3cccc(-c4ccc5ccccc5c4)c3-c3cc4ccccc4c4ccccc34)cc21 ; CC1(C)c2ccccc2-c2ccc(N(c3ccc(-c4ccc5ccccc5c4)cc3)c3cccc(-c4ccc5ccccc5c4)c3-c3cc4ccccc4c4ccccc34)cc21 ; CC1(C)c2ccccc2-c2ccc(N(c3ccccc3-c3ccccc3)c3cccc(-c4ccc5ccccc5c4)c3-c3cc4ccccc4c4ccccc34)cc21 ; CC1(C)c2ccccc2-c2ccc(N(c3ccccc3-c3ccc4ccccc4c3)c3cccc(-c4ccc5ccccc5c4)c3-c3cc4ccccc4c4ccccc34)cc21]

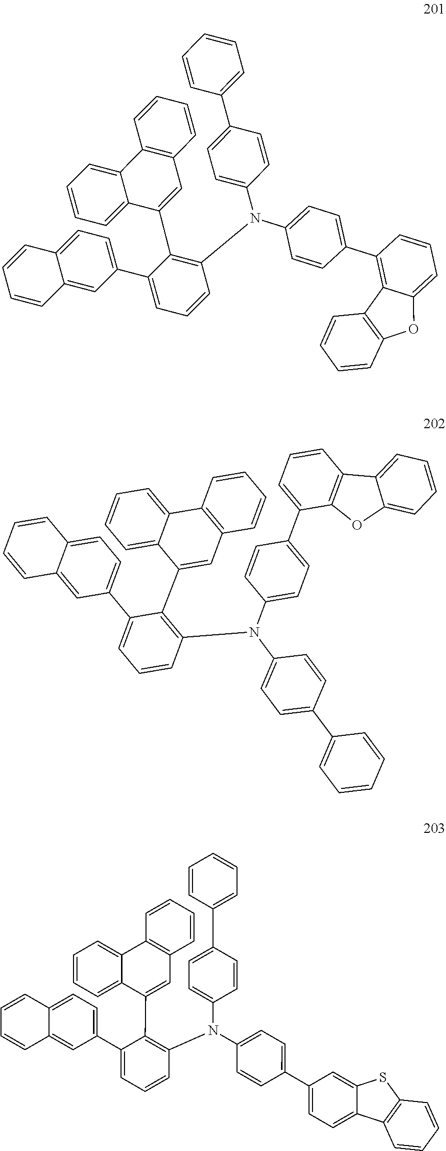 [SMILES: c1ccc(-c2ccc(N(c3ccc(-c4cccc5oc6ccccc6c45)cc3)c3cccc(-c4ccc5ccccc5c4)c3-c3cc4ccccc4c4ccccc34)cc2)cc1 ; c1ccc(-c2ccc(N(c3ccc(-c4cccc5c4oc4ccccc45)cc3)c3cccc(-c4ccc5ccccc5c4)c3-c3cc4ccccc4c4ccccc34)cc2)cc1 ; c1ccc(-c2ccc(N(c3ccc(-c4ccc5c(c4)sc4ccccc45)cc3)c3cccc(-c4ccc5ccccc5c4)c3-c3cc4ccccc4c4ccccc34)cc2)cc1]

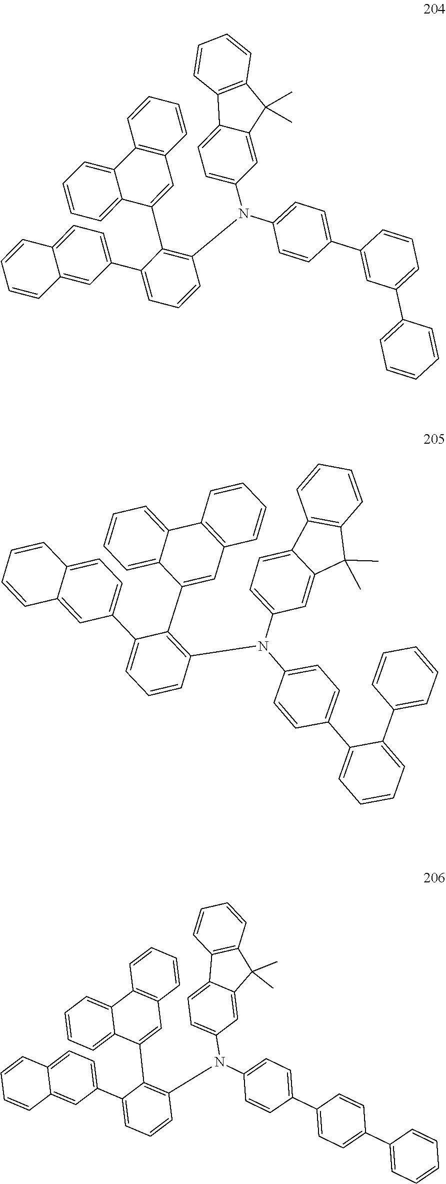 [SMILES: CC1(C)c2ccccc2-c2ccc(N(c3ccc(-c4cccc(-c5ccccc5)c4)cc3)c3cccc(-c4ccc5ccccc5c4)c3-c3cc4ccccc4c4ccccc34)cc21 ; CC1(C)c2ccccc2-c2ccc(N(c3ccc(-c4ccccc4-c4ccccc4)cc3)c3cccc(-c4ccc5ccccc5c4)c3-c3cc4ccccc4c4ccccc34)cc21 ; CC1(C)c2ccccc2-c2ccc(N(c3ccc(-c4ccc(-c5ccccc5)cc4)cc3)c3cccc(-c4ccc5ccccc5c4)c3-c3cc4ccccc4c4ccccc34)cc21]

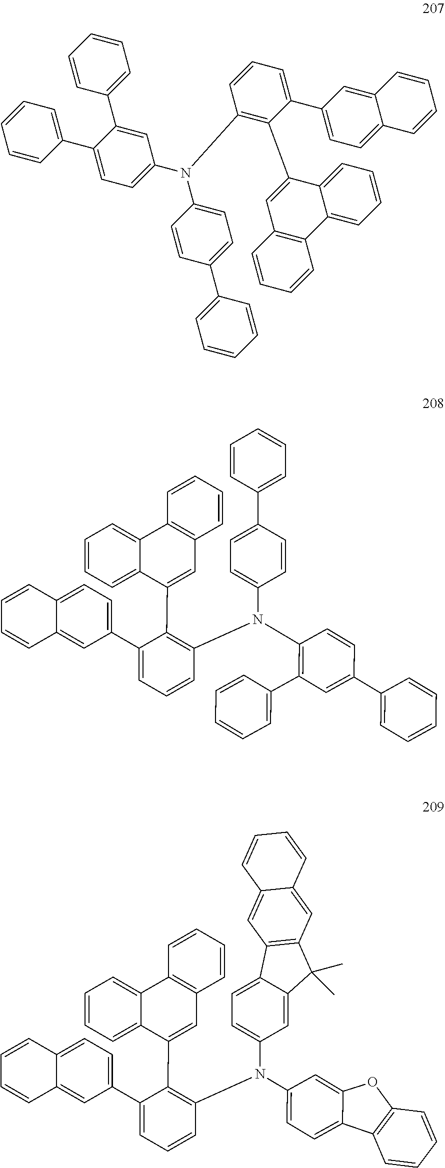 [SMILES: c1ccc(-c2ccc(N(c3ccc(-c4ccccc4)c(-c4ccccc4)c3)c3cccc(-c4ccc5ccccc5c4)c3-c3cc4ccccc4c4ccccc34)cc2)cc1 ; c1ccc(-c2ccc(N(c3ccc(-c4ccccc4)cc3-c3ccccc3)c3cccc(-c4ccc5ccccc5c4)c3-c3cc4ccccc4c4ccccc34)cc2)cc1 ; CC1(C)c2cc(N(c3ccc4c(c3)oc3ccccc34)c3cccc(-c4ccc5ccccc5c4)c3-c3cc4ccccc4c4ccccc34)ccc2-c2cc3ccccc3cc21]

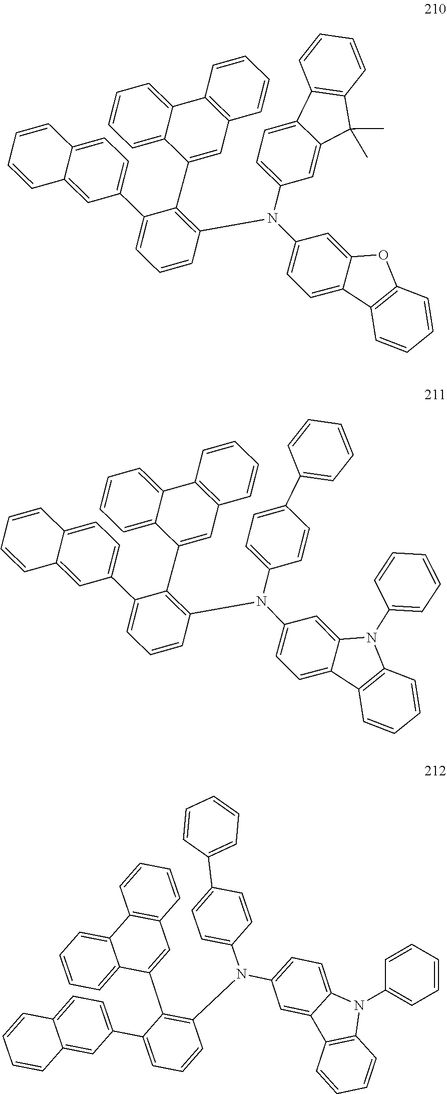 [SMILES: CC1(C)c2ccccc2-c2ccc(N(c3ccc4c(c3)oc3ccccc34)c3cccc(-c4ccc5ccccc5c4)c3-c3cc4ccccc4c4ccccc34)cc21 ; c1ccc(-c2ccc(N(c3ccc4c5ccccc5n(-c5ccccc5)c4c3)c3cccc(-c4ccc5ccccc5c4)c3-c3cc4ccccc4c4ccccc34)cc2)cc1 ; c1ccc(-c2ccc(N(c3ccc4c(c3)c3ccccc3n4-c3ccccc3)c3cccc(-c4ccc5ccccc5c4)c3-c3cc4ccccc4c4ccccc34)cc2)cc1]

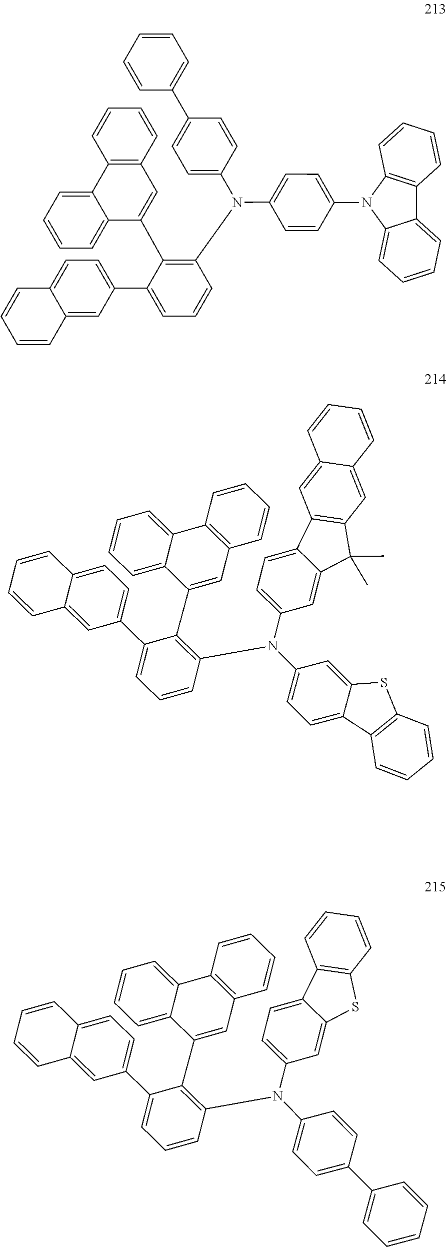 [SMILES: c1ccc(-c2ccc(N(c3ccc(-n4c5ccccc5c5ccccc54)cc3)c3cccc(-c4ccc5ccccc5c4)c3-c3cc4ccccc4c4ccccc34)cc2)cc1 ; CC1(C)c2cc(N(c3ccc4c(c3)sc3ccccc34)c3cccc(-c4ccc5ccccc5c4)c3-c3cc4ccccc4c4ccccc34)ccc2-c2cc3ccccc3cc21 ; c1ccc(-c2ccc(N(c3ccc4c(c3)sc3ccccc34)c3cccc(-c4ccc5ccccc5c4)c3-c3cc4ccccc4c4ccccc34)cc2)cc1]

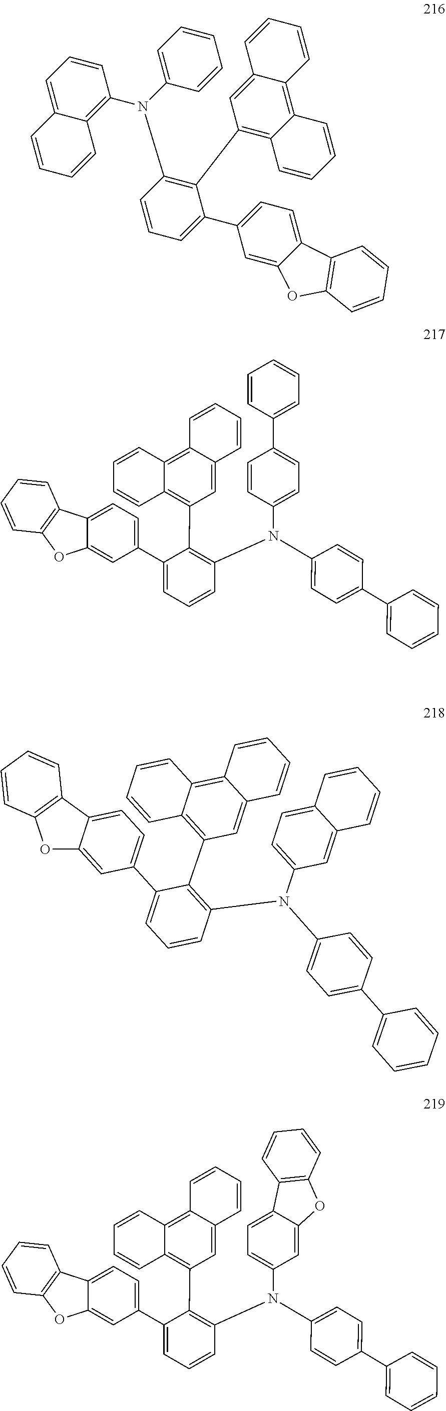 [SMILES: c1ccc(N(c2cccc(-c3ccc4c(c3)oc3ccccc34)c2-c2cc3ccccc3c3ccccc23)c2cccc3ccccc23)cc1 ; c1ccc(-c2ccc(N(c3ccc(-c4ccccc4)cc3)c3cccc(-c4ccc5c(c4)oc4ccccc45)c3-c3cc4ccccc4c4ccccc34)cc2)cc1 ; c1ccc(-c2ccc(N(c3ccc4ccccc4c3)c3cccc(-c4ccc5c(c4)oc4ccccc45)c3-c3cc4ccccc4c4ccccc34)cc2)cc1 ; c1ccc(-c2ccc(N(c3ccc4c(c3)oc3ccccc34)c3cccc(-c4ccc5c(c4)oc4ccccc45)c3-c3cc4ccccc4c4ccccc34)cc2)cc1]

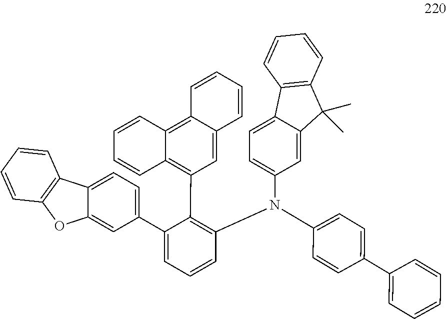 [SMILES: CC1(C)c2ccccc2-c2ccc(N(c3ccc(-c4ccccc4)cc3)c3cccc(-c4ccc5c(c4)oc4ccccc45)c3-c3cc4ccccc4c4ccccc34)cc21]

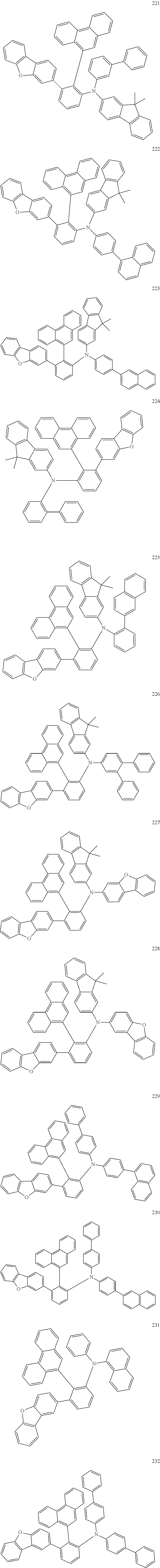

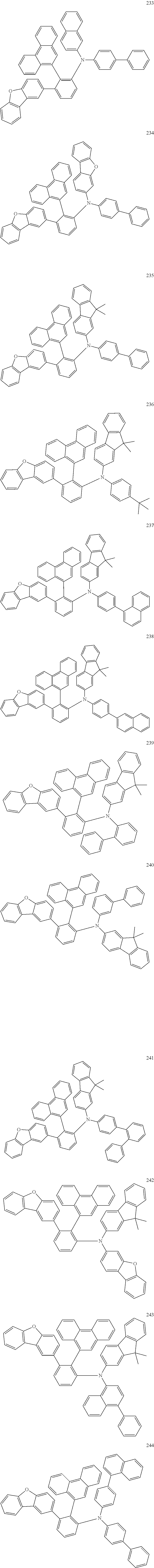

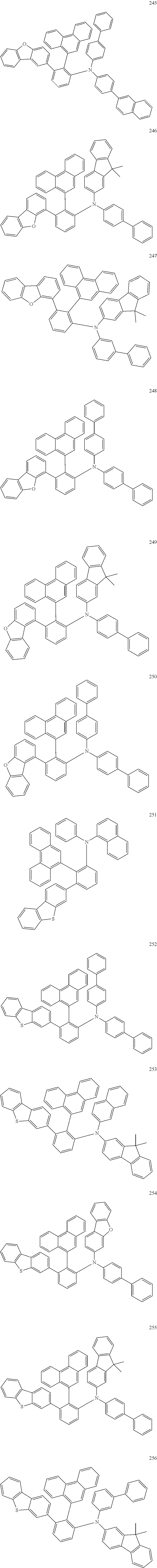

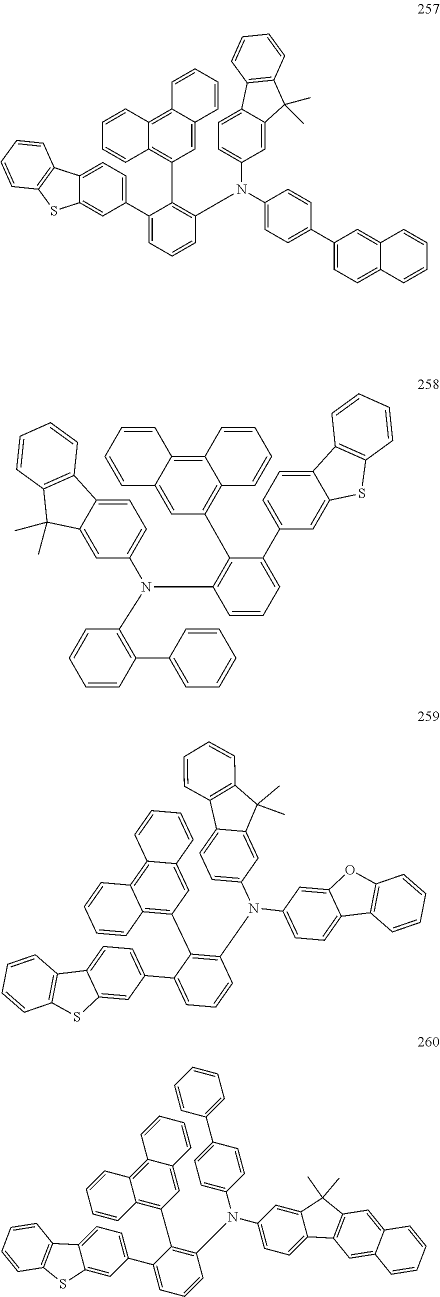 [SMILES: CC1(C)c2ccccc2-c2ccc(N(c3ccc(-c4ccc5ccccc5c4)cc3)c3cccc(-c4ccc5c(c4)sc4ccccc45)c3-c3cc4ccccc4c4ccccc34)cc21 ; CC1(C)c2ccccc2-c2ccc(N(c3ccccc3-c3ccccc3)c3cccc(-c4ccc5c(c4)sc4ccccc45)c3-c3cc4ccccc4c4ccccc34)cc21 ; CC1(C)c2ccccc2-c2ccc(N(c3ccc4c(c3)oc3ccccc34)c3cccc(-c4ccc5c(c4)sc4ccccc45)c3-c3cc4ccccc4c4ccccc34)cc21 ; CC1(C)c2cc(N(c3ccc(-c4ccccc4)cc3)c3cccc(-c4ccc5c(c4)sc4ccccc45)c3-c3cc4ccccc4c4ccccc34)ccc2-c2cc3ccccc3cc21]

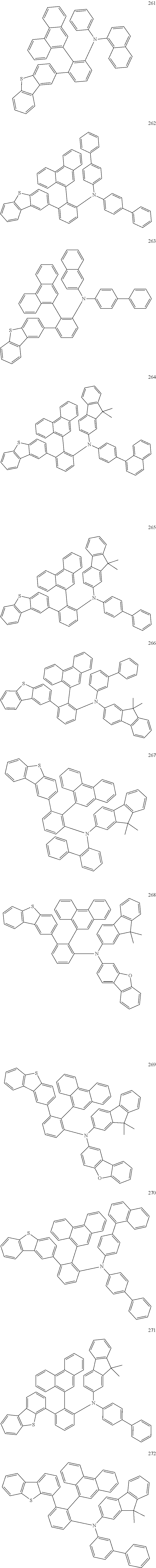

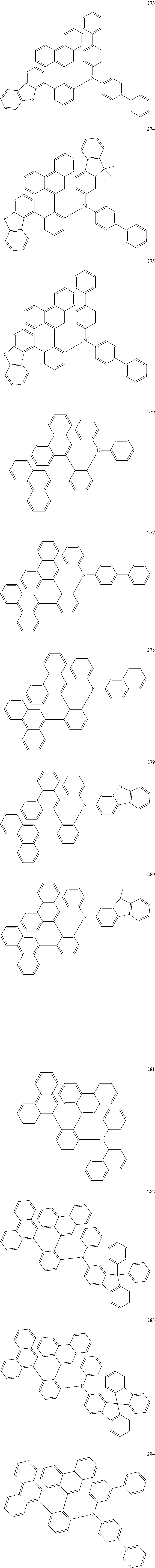

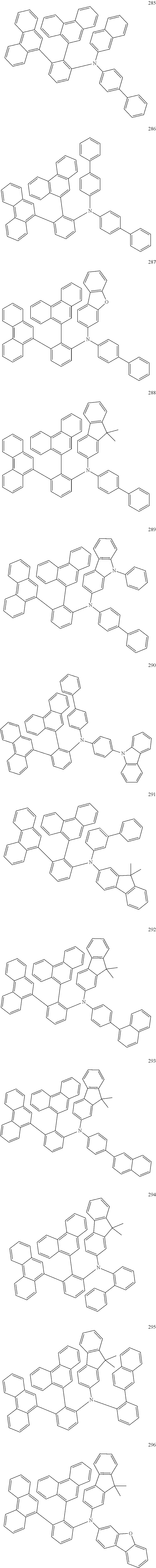

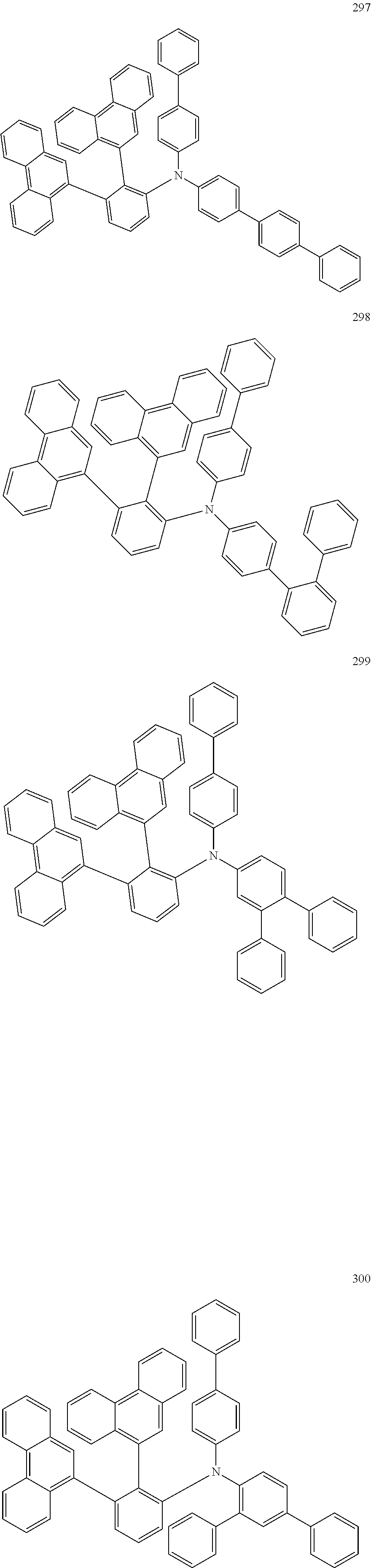 [SMILES: c1ccc(-c2ccc(-c3ccc(N(c4ccc(-c5ccccc5)cc4)c4cccc(-c5cc6ccccc6c6ccccc56)c4-c4cc5ccccc5c5ccccc45)cc3)cc2)cc1 ; c1ccc(-c2ccc(N(c3ccc(-c4ccccc4-c4ccccc4)cc3)c3cccc(-c4cc5ccccc5c5ccccc45)c3-c3cc4ccccc4c4ccccc34)cc2)cc1 ; c1ccc(-c2ccc(N(c3ccc(-c4ccccc4)c(-c4ccccc4)c3)c3cccc(-c4cc5ccccc5c5ccccc45)c3-c3cc4ccccc4c4ccccc34)cc2)cc1 ; c1ccc(-c2ccc(N(c3ccc(-c4ccccc4)cc3-c3ccccc3)c3cccc(-c4cc5ccccc5c5ccccc45)c3-c3cc4ccccc4c4ccccc34)cc2)cc1]

[chemical structure]

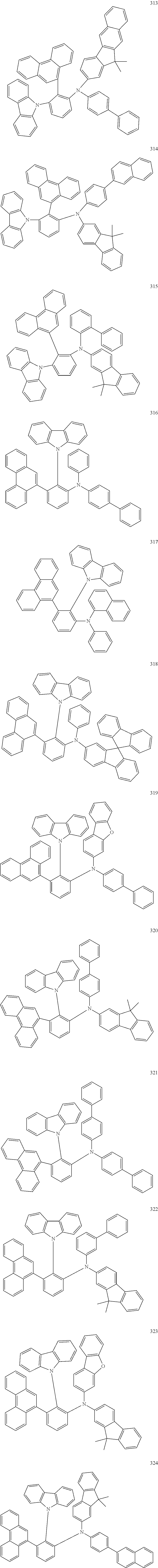

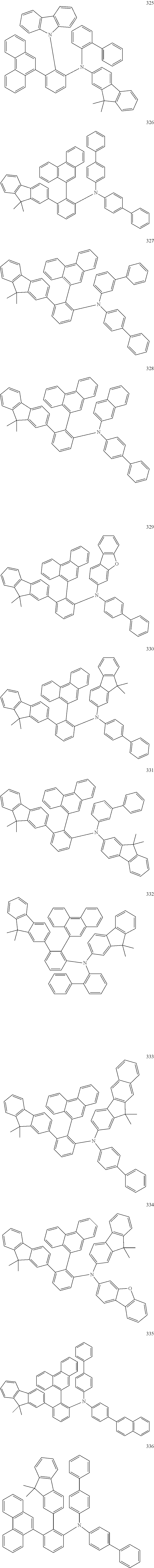

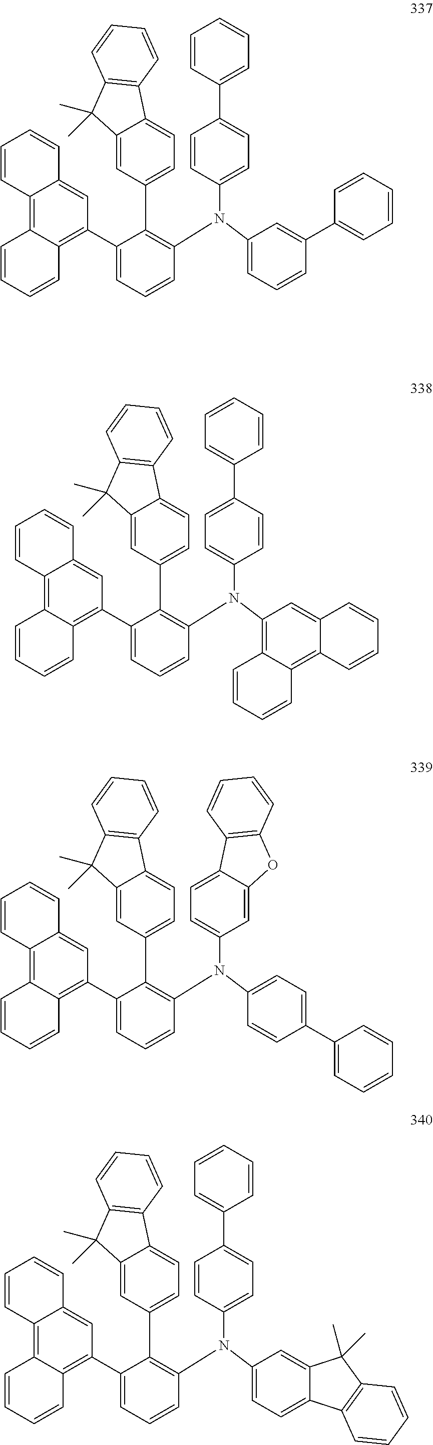 [SMILES: CC1(C)c2ccccc2-c2ccc(-c3c(-c4cc5ccccc5c5ccccc45)cccc3N(c3ccc(-c4ccccc4)cc3)c3cccc(-c4ccccc4)c3)cc21 ; CC1(C)c2ccccc2-c2ccc(-c3c(-c4cc5ccccc5c5ccccc45)cccc3N(c3ccc(-c4ccccc4)cc3)c3cc4ccccc4c4ccccc34)cc21 ; CC1(C)c2ccccc2-c2ccc(-c3c(-c4cc5ccccc5c5ccccc45)cccc3N(c3ccc(-c4ccccc4)cc3)c3ccc4c(c3)oc3ccccc34)cc21 ; CC1(C)c2ccccc2-c2ccc(-c3c(-c4cc5ccccc5c5ccccc45)cccc3N(c3ccc(-c4ccccc4)cc3)c3ccc4c(c3)C(C)(C)c3ccccc3-4)cc21]

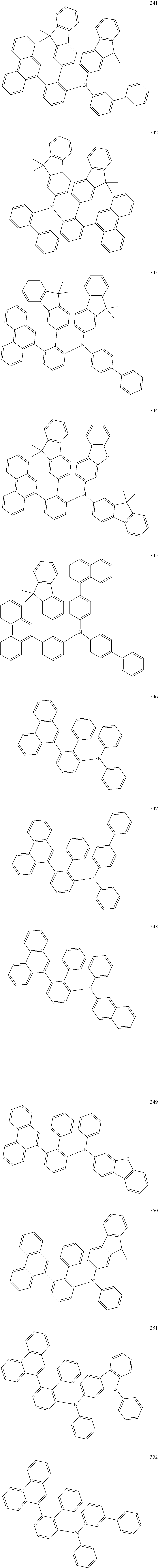

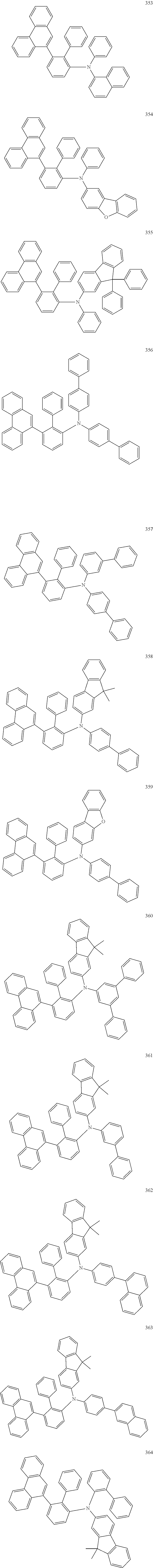

[chemical structure]

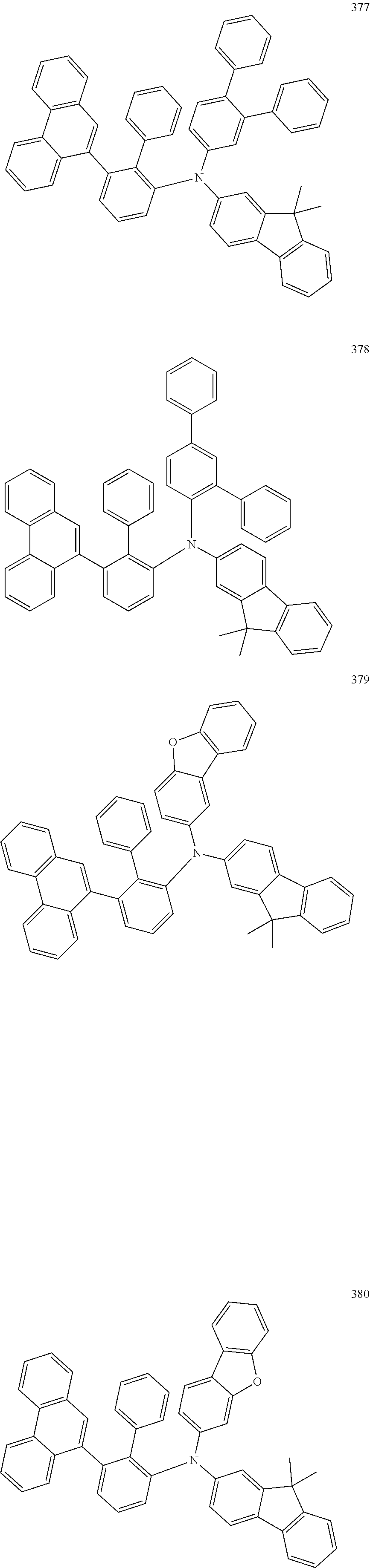 [SMILES: CC1(C)c2ccccc2-c2ccc(N(c3ccc(-c4ccccc4)c(-c4ccccc4)c3)c3cccc(-c4cc5ccccc5c5ccccc45)c3-c3ccccc3)cc21 ; CC1(C)c2ccccc2-c2ccc(N(c3ccc(-c4ccccc4)cc3-c3ccccc3)c3cccc(-c4cc5ccccc5c5ccccc45)c3-c3ccccc3)cc21 ; CC1(C)c2ccccc2-c2ccc(N(c3ccc4oc5ccccc5c4c3)c3cccc(-c4cc5ccccc5c5ccccc45)c3-c3ccccc3)cc21 ; CC1(C)c2ccccc2-c2ccc(N(c3ccc4c(c3)oc3ccccc34)c3cccc(-c4cc5ccccc5c5ccccc45)c3-c3ccccc3)cc21]

[chemical structure]

[chemical structure]

[chemical structure]

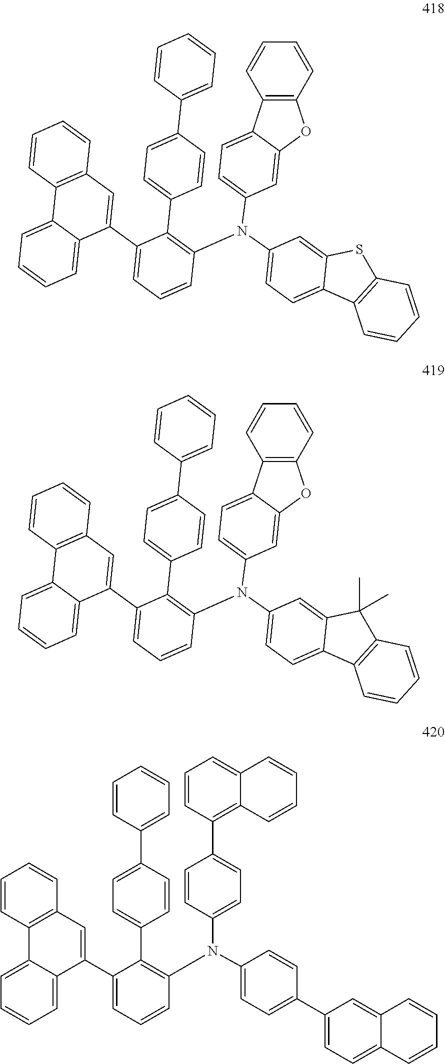 [SMILES: c1ccc(-c2ccc(-c3c(-c4cc5ccccc5c5ccccc45)cccc3N(c3ccc4c(c3)oc3ccccc34)c3ccc4c(c3)sc3ccccc34)cc2)cc1 ; CC1(C)c2ccccc2-c2ccc(N(c3ccc4c(c3)oc3ccccc34)c3cccc(-c4cc5ccccc5c5ccccc45)c3-c3ccc(-c4ccccc4)cc3)cc21 ; c1ccc(-c2ccc(-c3c(-c4cc5ccccc5c5ccccc45)cccc3N(c3ccc(-c4ccc5ccccc5c4)cc3)c3ccc(-c4cccc5ccccc45)cc3)cc2)cc1]

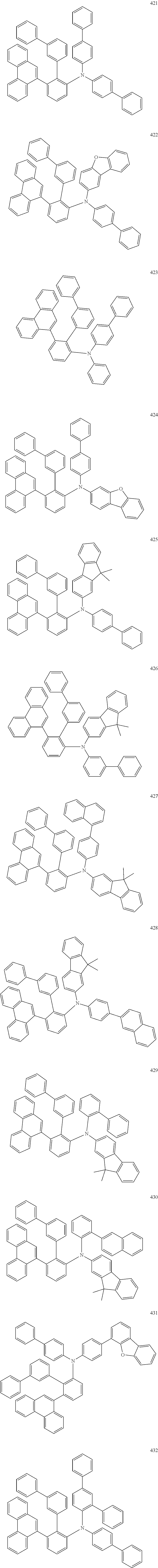

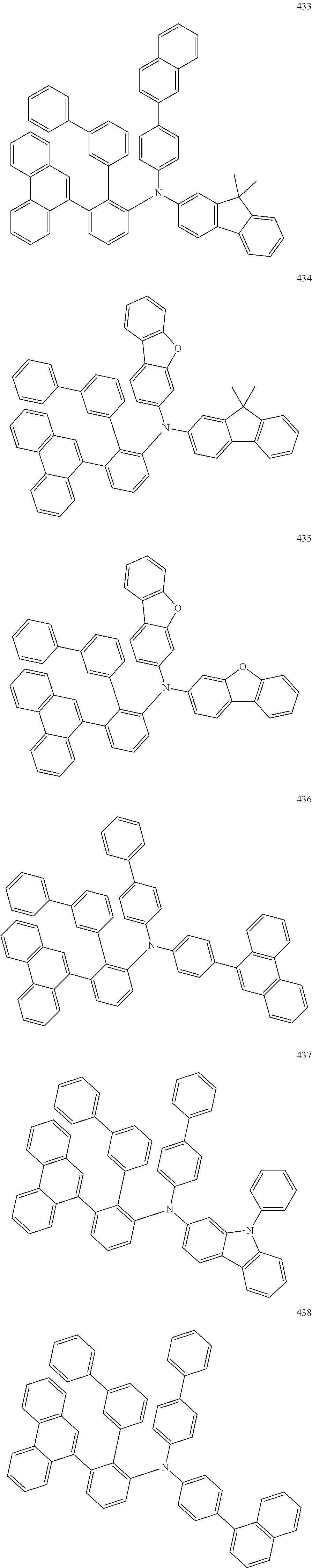

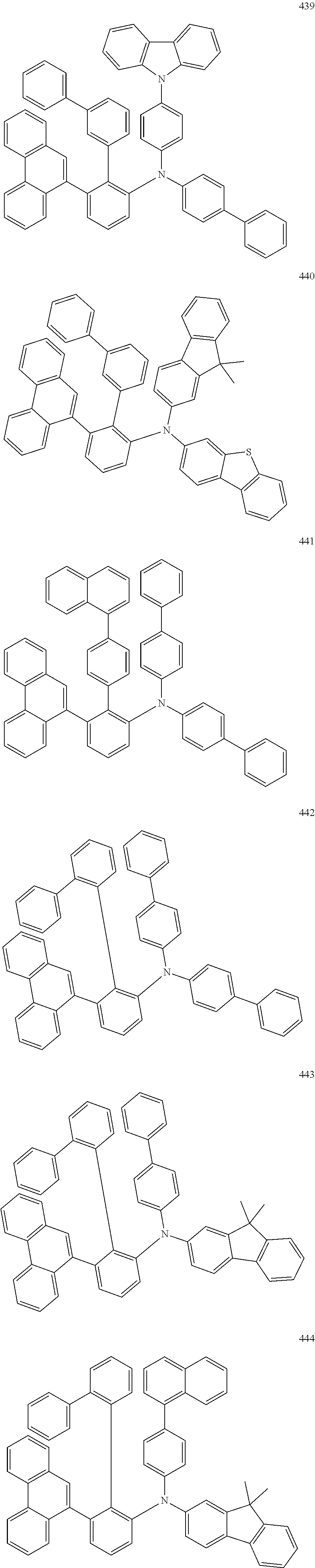

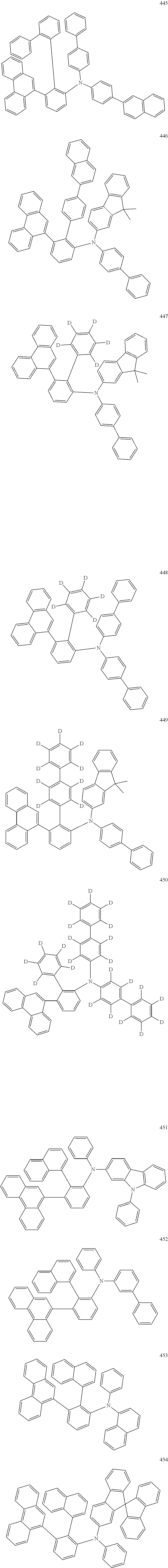

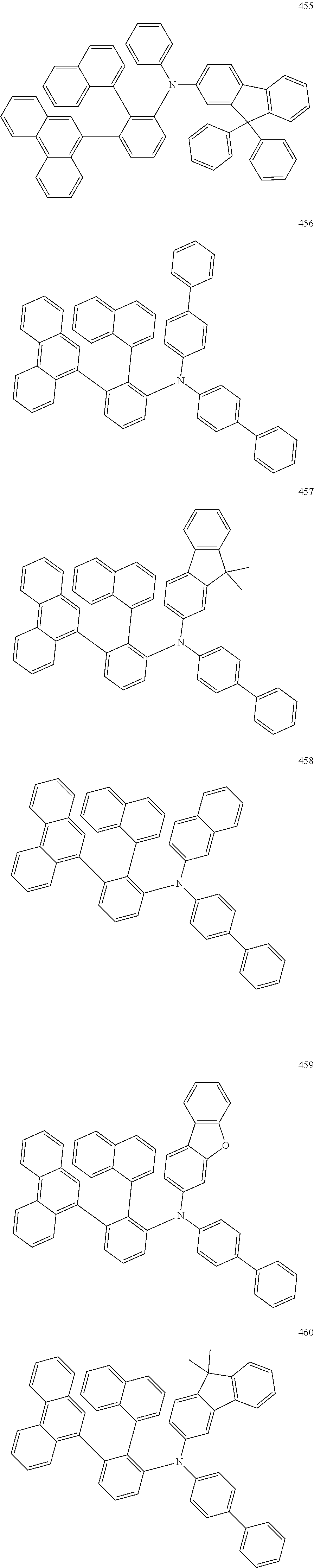

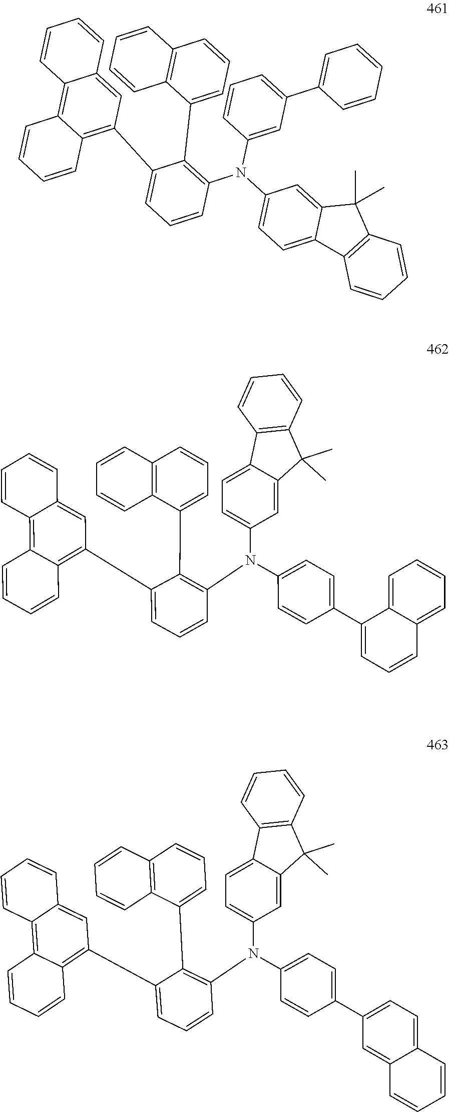 [SMILES: CC1(C)c2ccccc2-c2ccc(N(c3cccc(-c4ccccc4)c3)c3cccc(-c4cc5ccccc5c5ccccc45)c3-c3cccc4ccccc34)cc21 ; CC1(C)c2ccccc2-c2ccc(N(c3ccc(-c4cccc5ccccc45)cc3)c3cccc(-c4cc5ccccc5c5ccccc45)c3-c3cccc4ccccc34)cc21 ; CC1(C)c2ccccc2-c2ccc(N(c3ccc(-c4ccc5ccccc5c4)cc3)c3cccc(-c4cc5ccccc5c5ccccc45)c3-c3cccc4ccccc34)cc21]

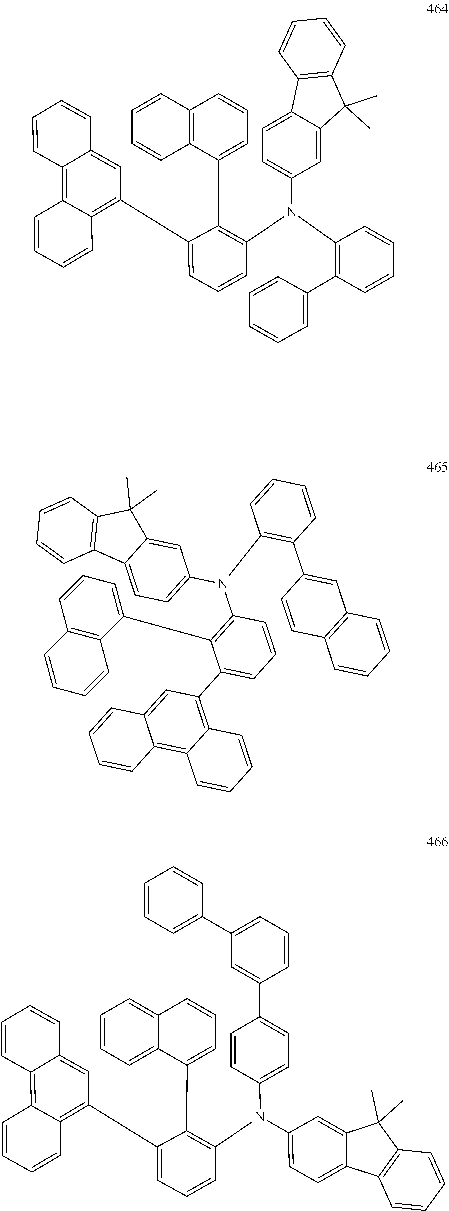 [SMILES: CC1(C)c2ccccc2-c2ccc(N(c3ccccc3-c3ccccc3)c3cccc(-c4cc5ccccc5c5ccccc45)c3-c3cccc4ccccc34)cc21 ; CC1(C)c2ccccc2-c2ccc(N(c3ccccc3-c3ccc4ccccc4c3)c3cccc(-c4cc5ccccc5c5ccccc45)c3-c3cccc4ccccc34)cc21 ; CC1(C)c2ccccc2-c2ccc(N(c3ccc(-c4cccc(-c5ccccc5)c4)cc3)c3cccc(-c4cc5ccccc5c5ccccc45)c3-c3cccc4ccccc34)cc21]

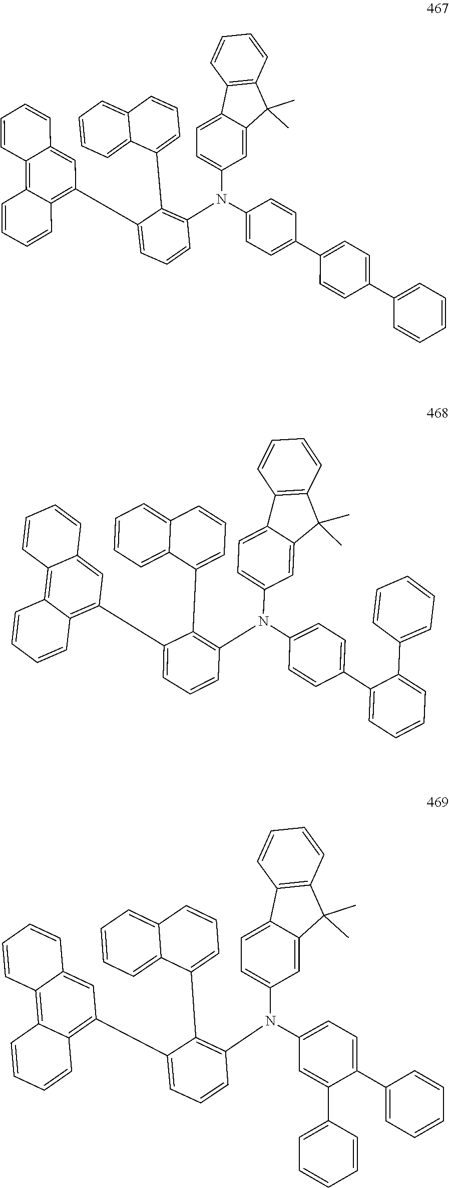 [SMILES: CC1(C)c2ccccc2-c2ccc(N(c3ccc(-c4ccc(-c5ccccc5)cc4)cc3)c3cccc(-c4cc5ccccc5c5ccccc45)c3-c3cccc4ccccc34)cc21 ; CC1(C)c2ccccc2-c2ccc(N(c3ccc(-c4ccccc4-c4ccccc4)cc3)c3cccc(-c4cc5ccccc5c5ccccc45)c3-c3cccc4ccccc34)cc21 ; CC1(C)c2ccccc2-c2ccc(N(c3ccc(-c4ccccc4)c(-c4ccccc4)c3)c3cccc(-c4cc5ccccc5c5ccccc45)c3-c3cccc4ccccc34)cc21]

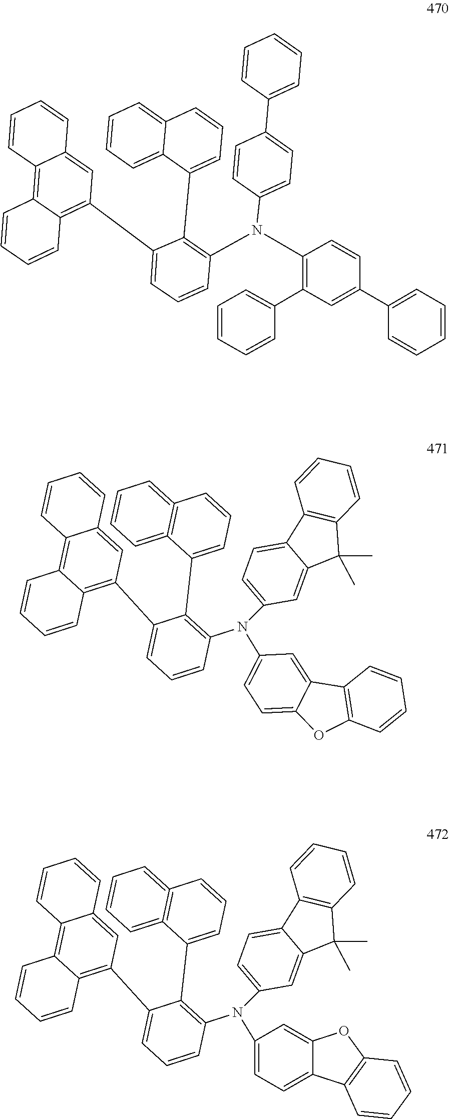

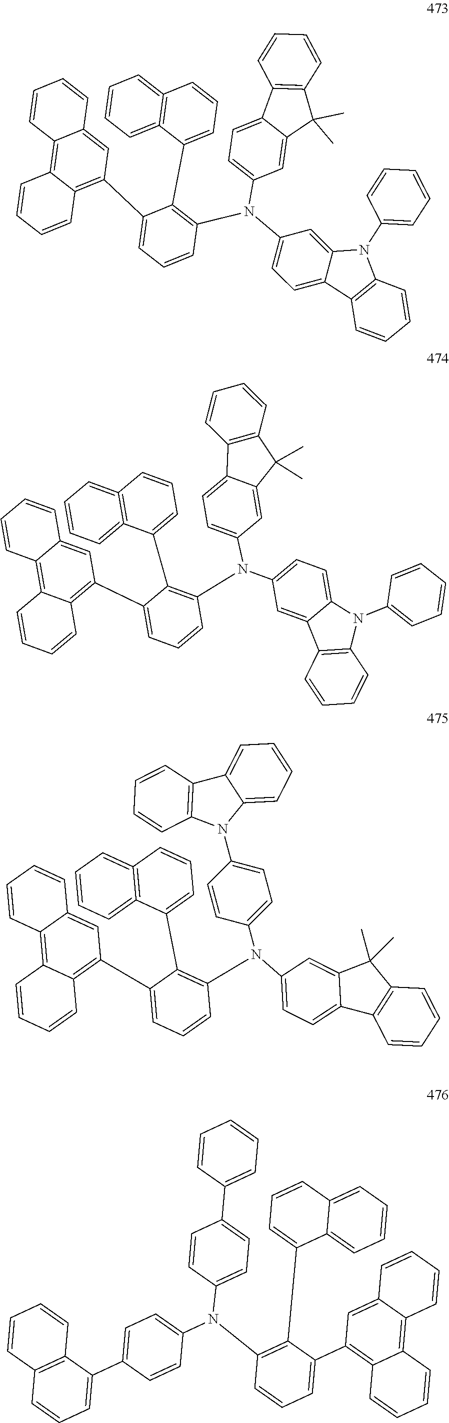

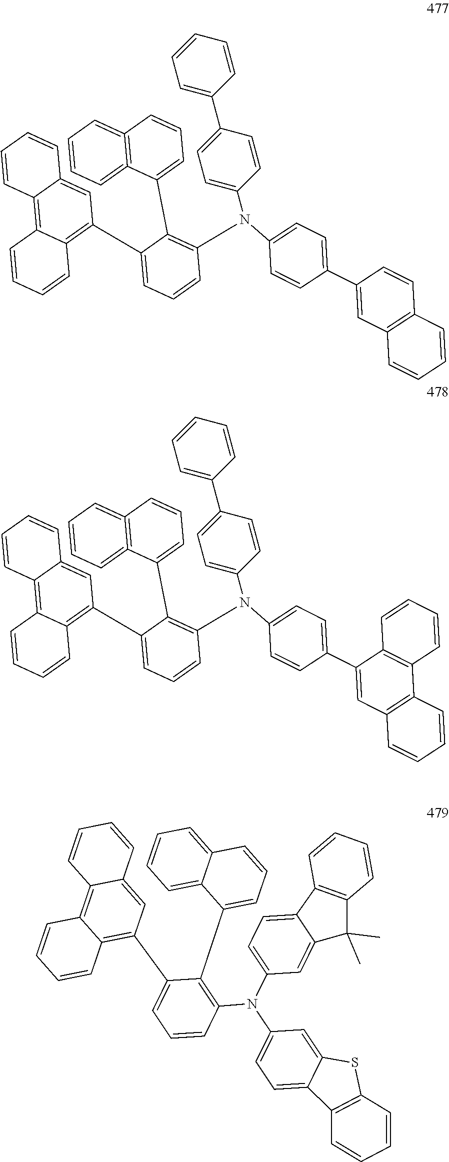

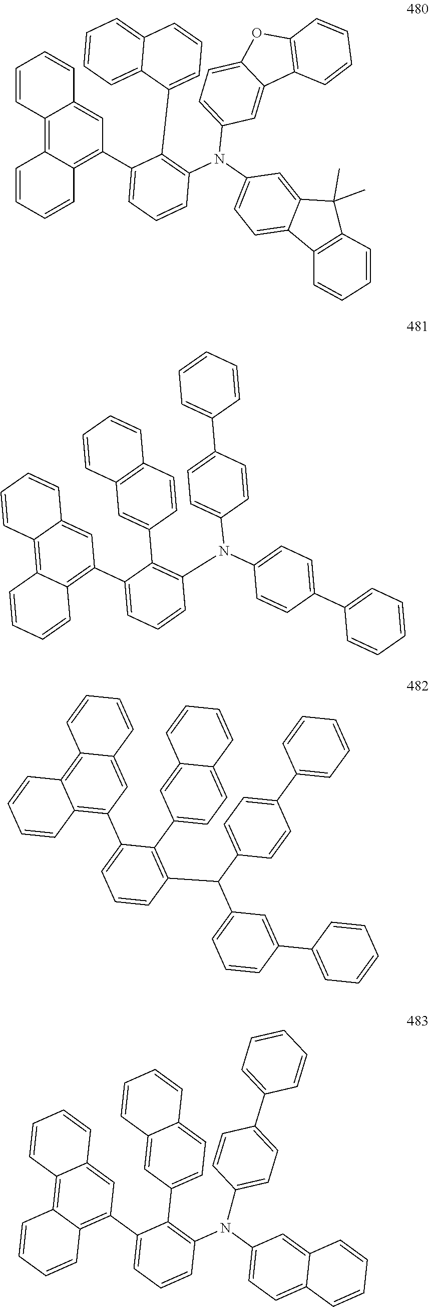

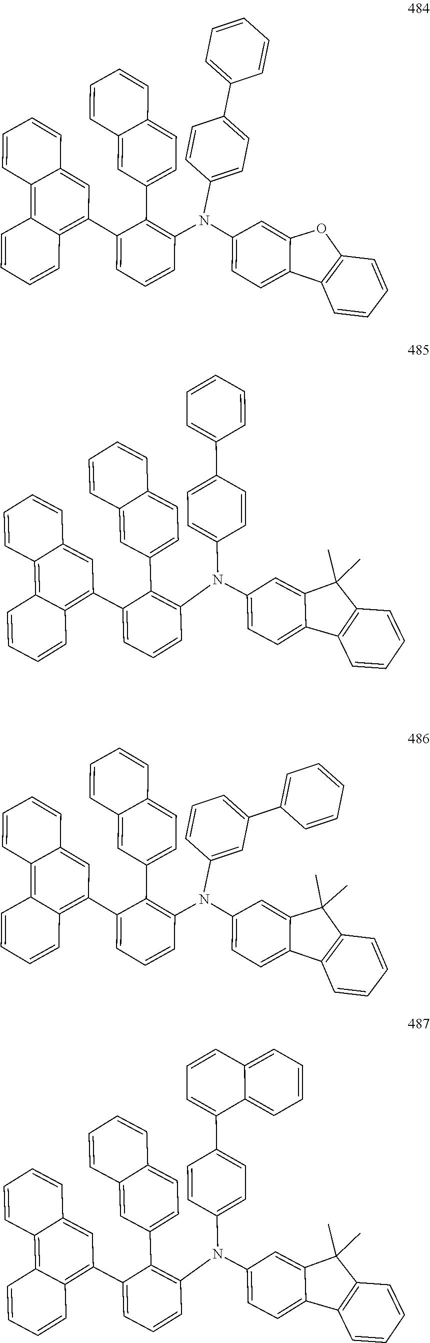 [SMILES: c1ccc(-c2ccc(N(c3ccc4c(c3)oc3ccccc34)c3cccc(-c4cc5ccccc5c5ccccc45)c3-c3ccc4ccccc4c3)cc2)cc1 ; CC1(C)c2ccccc2-c2ccc(N(c3ccc(-c4ccccc4)cc3)c3cccc(-c4cc5ccccc5c5ccccc45)c3-c3ccc4ccccc4c3)cc21 ; CC1(C)c2ccccc2-c2ccc(N(c3cccc(-c4ccccc4)c3)c3cccc(-c4cc5ccccc5c5ccccc45)c3-c3ccc4ccccc4c3)cc21 ; CC1(C)c2ccccc2-c2ccc(N(c3ccc(-c4cccc5ccccc45)cc3)c3cccc(-c4cc5ccccc5c5ccccc45)c3-c3ccc4ccccc4c3)cc21]

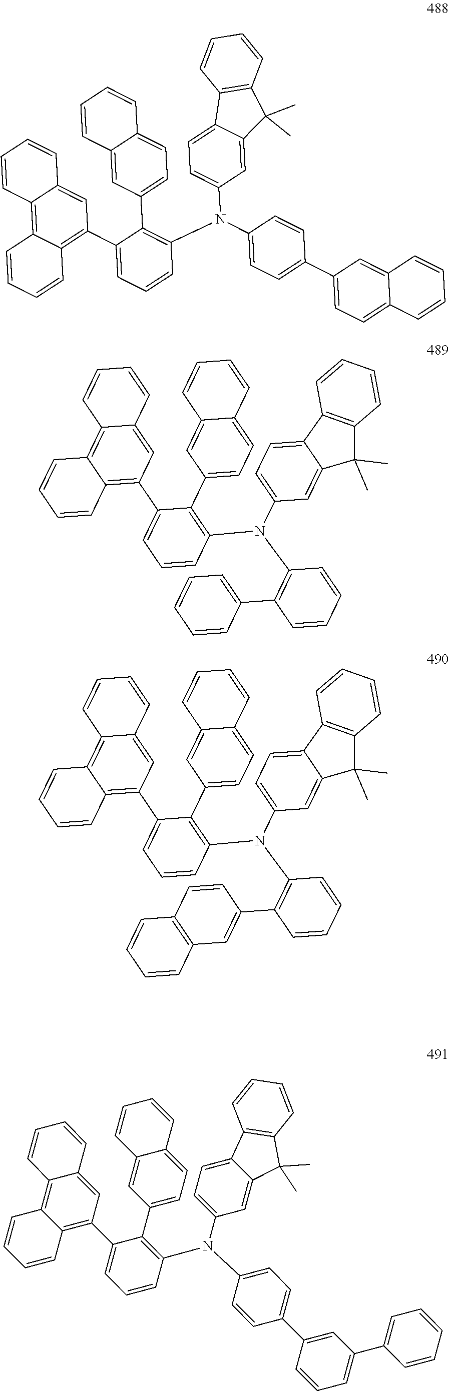

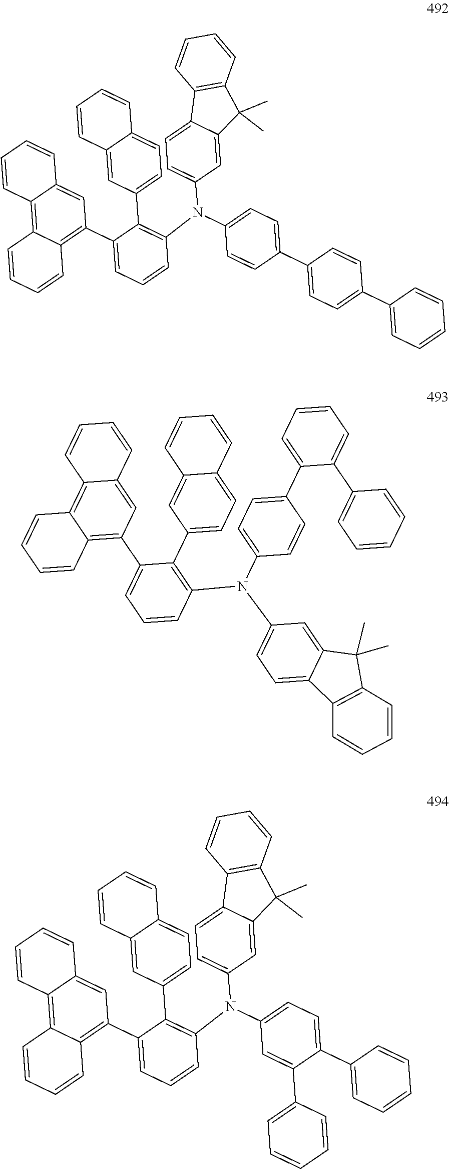 [SMILES: CC1(C)c2ccccc2-c2ccc(N(c3ccc(-c4ccc(-c5ccccc5)cc4)cc3)c3cccc(-c4cc5ccccc5c5ccccc45)c3-c3ccc4ccccc4c3)cc21 ; CC1(C)c2ccccc2-c2ccc(N(c3ccc(-c4ccccc4-c4ccccc4)cc3)c3cccc(-c4cc5ccccc5c5ccccc45)c3-c3ccc4ccccc4c3)cc21 ; CC1(C)c2ccccc2-c2ccc(N(c3ccc(-c4ccccc4)c(-c4ccccc4)c3)c3cccc(-c4cc5ccccc5c5ccccc45)c3-c3ccc4ccccc4c3)cc21]

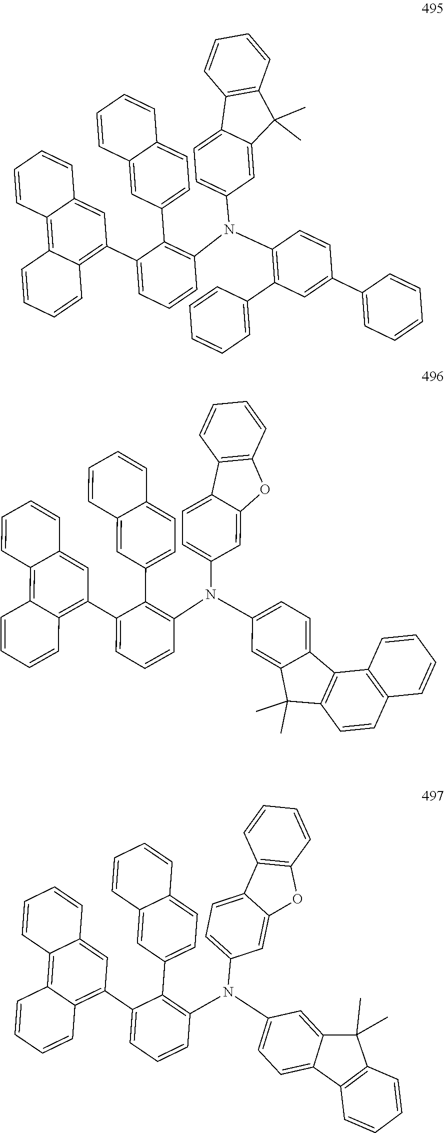 [SMILES: CC1(C)c2ccccc2-c2ccc(N(c3ccc(-c4ccccc4)cc3-c3ccccc3)c3cccc(-c4cc5ccccc5c5ccccc45)c3-c3ccc4ccccc4c3)cc21 ; CC1(C)c2cc(N(c3ccc4c(c3)oc3ccccc34)c3cccc(-c4cc5ccccc5c5ccccc45)c3-c3ccc4ccccc4c3)ccc2-c2c1ccc1ccccc21 ; CC1(C)c2ccccc2-c2ccc(N(c3ccc4c(c3)oc3ccccc34)c3cccc(-c4cc5ccccc5c5ccccc45)c3-c3ccc4ccccc4c3)cc21]

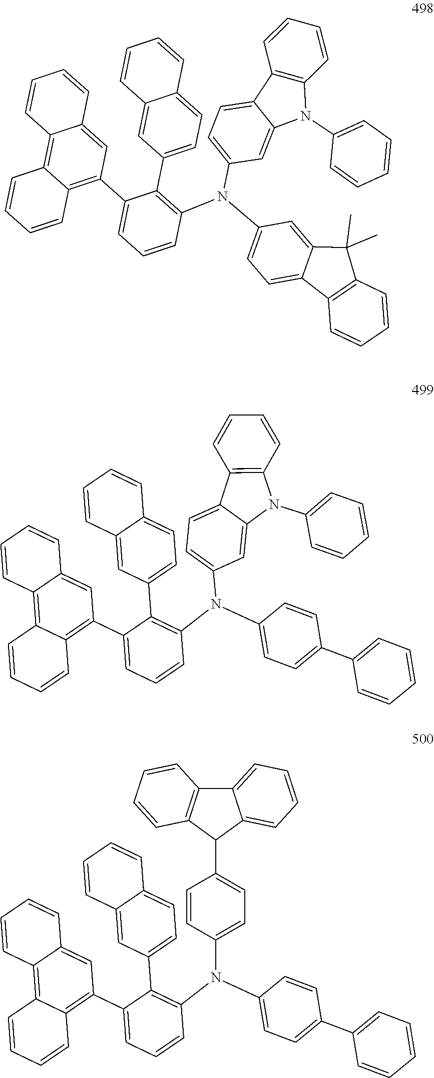 [SMILES: CC1(C)c2ccccc2-c2ccc(N(c3ccc4c5ccccc5n(-c5ccccc5)c4c3)c3cccc(-c4cc5ccccc5c5ccccc45)c3-c3ccc4ccccc4c3)cc21 ; c1ccc(-c2ccc(N(c3ccc4c5ccccc5n(-c5ccccc5)c4c3)c3cccc(-c4cc5ccccc5c5ccccc45)c3-c3ccc4ccccc4c3)cc2)cc1 ; c1ccc(-c2ccc(N(c3ccc(C4c5ccccc5-c5ccccc54)cc3)c3cccc(-c4cc5ccccc5c5ccccc45)c3-c3ccc4ccccc4c3)cc2)cc1]

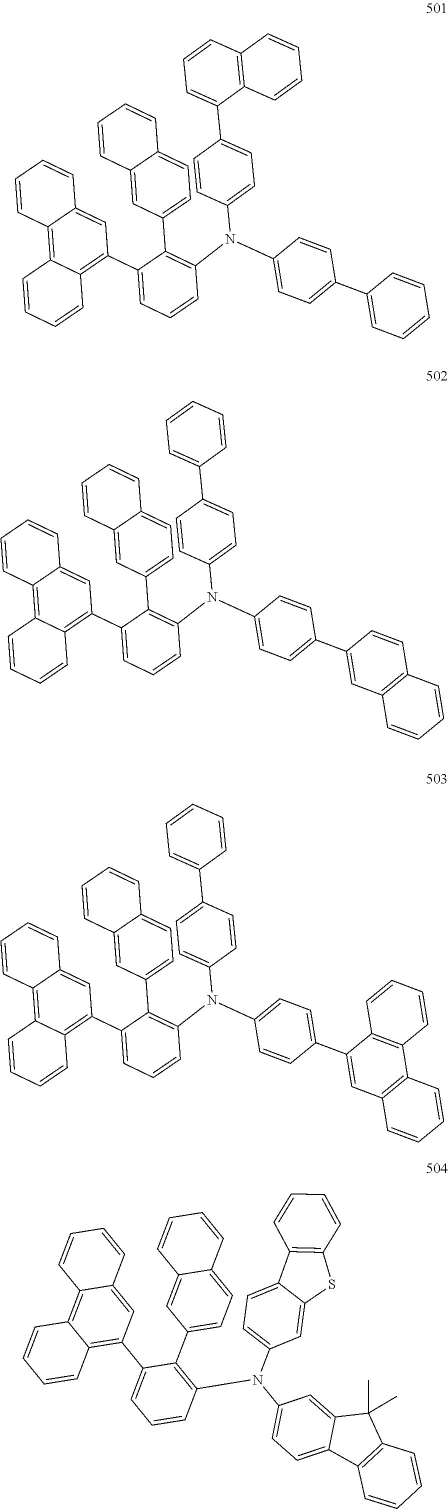 [SMILES: c1ccc(-c2ccc(N(c3ccc(-c4cccc5ccccc45)cc3)c3cccc(-c4cc5ccccc5c5ccccc45)c3-c3ccc4ccccc4c3)cc2)cc1 ; c1ccc(-c2ccc(N(c3ccc(-c4ccc5ccccc5c4)cc3)c3cccc(-c4cc5ccccc5c5ccccc45)c3-c3ccc4ccccc4c3)cc2)cc1 ; c1ccc(-c2ccc(N(c3ccc(-c4cc5ccccc5c5ccccc45)cc3)c3cccc(-c4cc5ccccc5c5ccccc45)c3-c3ccc4ccccc4c3)cc2)cc1 ; CC1(C)c2ccccc2-c2ccc(N(c3ccc4c(c3)sc3ccccc34)c3cccc(-c4cc5ccccc5c5ccccc45)c3-c3ccc4ccccc4c3)cc21]

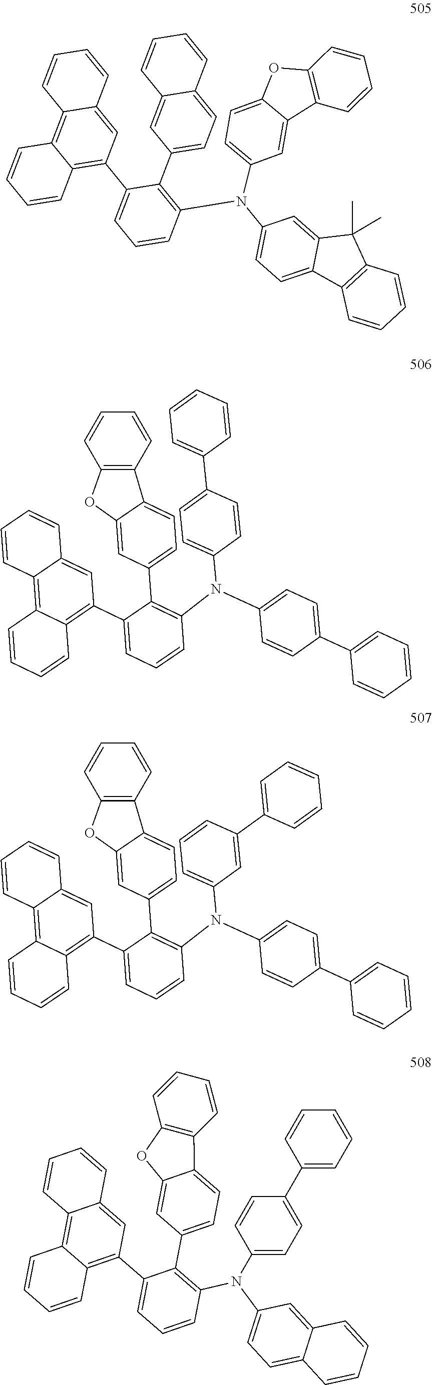 [SMILES: CC1(C)c2ccccc2-c2ccc(N(c3ccc4oc5ccccc5c4c3)c3cccc(-c4cc5ccccc5c5ccccc45)c3-c3ccc4ccccc4c3)cc21 ; c1ccc(-c2ccc(N(c3ccc(-c4ccccc4)cc3)c3cccc(-c4cc5ccccc5c5ccccc45)c3-c3ccc4c(c3)oc3ccccc34)cc2)cc1 ; c1ccc(-c2ccc(N(c3cccc(-c4ccccc4)c3)c3cccc(-c4cc5ccccc5c5ccccc45)c3-c3ccc4c(c3)oc3ccccc34)cc2)cc1 ; c1ccc(-c2ccc(N(c3ccc4ccccc4c3)c3cccc(-c4cc5ccccc5c5ccccc45)c3-c3ccc4c(c3)oc3ccccc34)cc2)cc1]

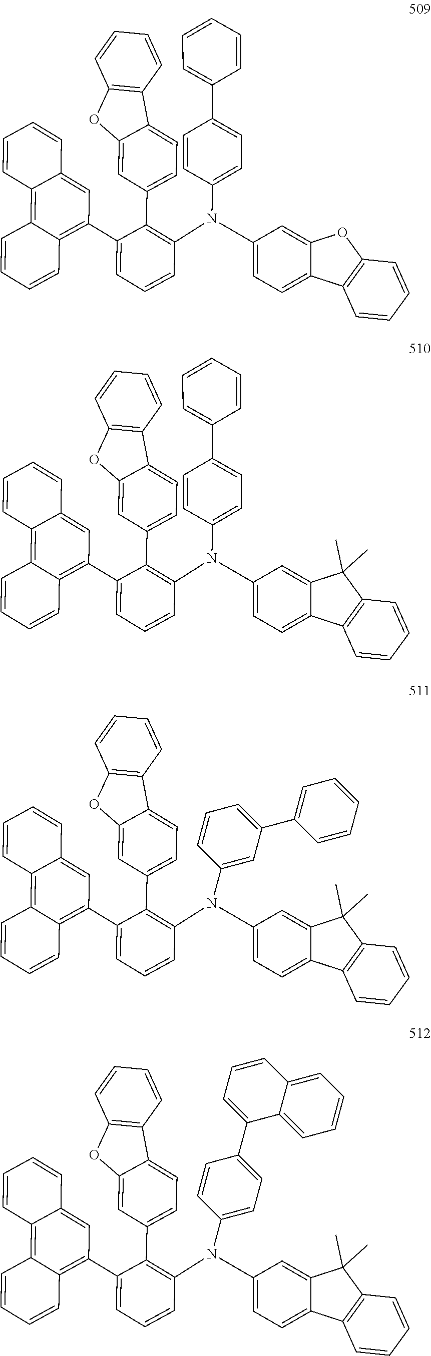 [SMILES: c1ccc(-c2ccc(N(c3ccc4c(c3)oc3ccccc34)c3cccc(-c4cc5ccccc5c5ccccc45)c3-c3ccc4c(c3)oc3ccccc34)cc2)cc1 ; CC1(C)c2ccccc2-c2ccc(N(c3ccc(-c4ccccc4)cc3)c3cccc(-c4cc5ccccc5c5ccccc45)c3-c3ccc4c(c3)oc3ccccc34)cc21 ; CC1(C)c2ccccc2-c2ccc(N(c3cccc(-c4ccccc4)c3)c3cccc(-c4cc5ccccc5c5ccccc45)c3-c3ccc4c(c3)oc3ccccc34)cc21 ; CC1(C)c2ccccc2-c2ccc(N(c3ccc(-c4cccc5ccccc45)cc3)c3cccc(-c4cc5ccccc5c5ccccc45)c3-c3ccc4c(c3)oc3ccccc34)cc21]

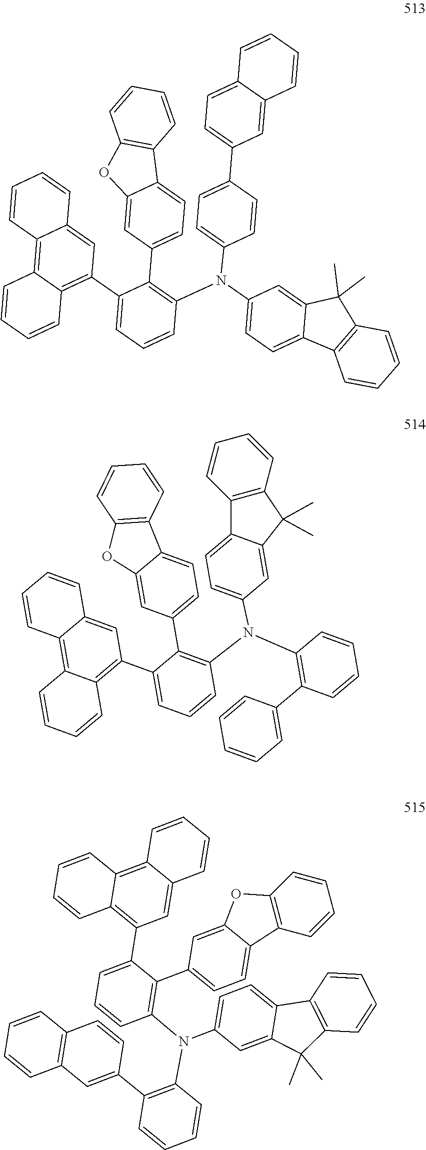 [SMILES: CC1(C)c2ccccc2-c2ccc(N(c3ccc(-c4ccc5ccccc5c4)cc3)c3cccc(-c4cc5ccccc5c5ccccc45)c3-c3ccc4c(c3)oc3ccccc34)cc21 ; CC1(C)c2ccccc2-c2ccc(N(c3ccccc3-c3ccccc3)c3cccc(-c4cc5ccccc5c5ccccc45)c3-c3ccc4c(c3)oc3ccccc34)cc21 ; CC1(C)c2ccccc2-c2ccc(N(c3ccccc3-c3ccc4ccccc4c3)c3cccc(-c4cc5ccccc5c5ccccc45)c3-c3ccc4c(c3)oc3ccccc34)cc21]

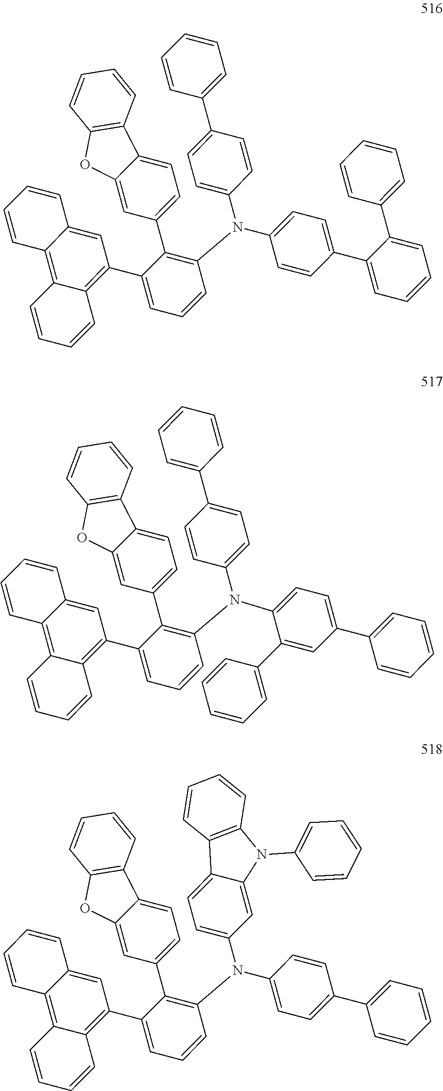 [SMILES: c1ccc(-c2ccc(N(c3ccc(-c4ccccc4-c4ccccc4)cc3)c3cccc(-c4cc5ccccc5c5ccccc45)c3-c3ccc4c(c3)oc3ccccc34)cc2)cc1 ; c1ccc(-c2ccc(N(c3ccc(-c4ccccc4)cc3-c3ccccc3)c3cccc(-c4cc5ccccc5c5ccccc45)c3-c3ccc4c(c3)oc3ccccc34)cc2)cc1 ; c1ccc(-c2ccc(N(c3ccc4c5ccccc5n(-c5ccccc5)c4c3)c3cccc(-c4cc5ccccc5c5ccccc45)c3-c3ccc4c(c3)oc3ccccc34)cc2)cc1]

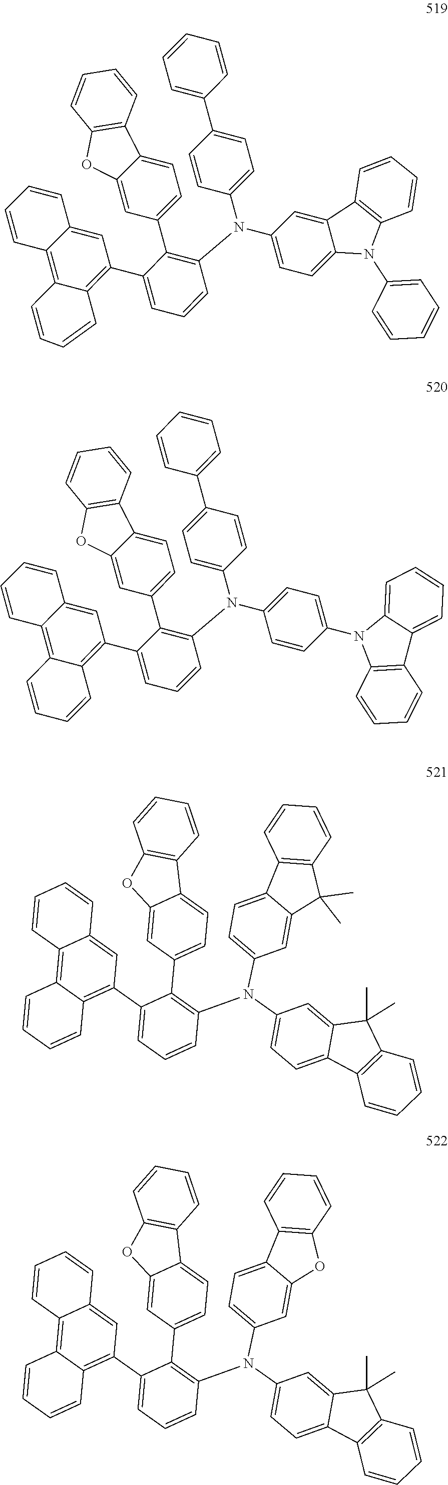 [SMILES: c1ccc(-c2ccc(N(c3ccc4c(c3)c3ccccc3n4-c3ccccc3)c3cccc(-c4cc5ccccc5c5ccccc45)c3-c3ccc4c(c3)oc3ccccc34)cc2)cc1 ; c1ccc(-c2ccc(N(c3ccc(-n4c5ccccc5c5ccccc54)cc3)c3cccc(-c4cc5ccccc5c5ccccc45)c3-c3ccc4c(c3)oc3ccccc34)cc2)cc1 ; CC1(C)c2ccccc2-c2ccc(N(c3ccc4c(c3)C(C)(C)c3ccccc3-4)c3cccc(-c4cc5ccccc5c5ccccc45)c3-c3ccc4c(c3)oc3ccccc34)cc21 ; CC1(C)c2ccccc2-c2ccc(N(c3ccc4c(c3)oc3ccccc34)c3cccc(-c4cc5ccccc5c5ccccc45)c3-c3ccc4c(c3)oc3ccccc34)cc21]

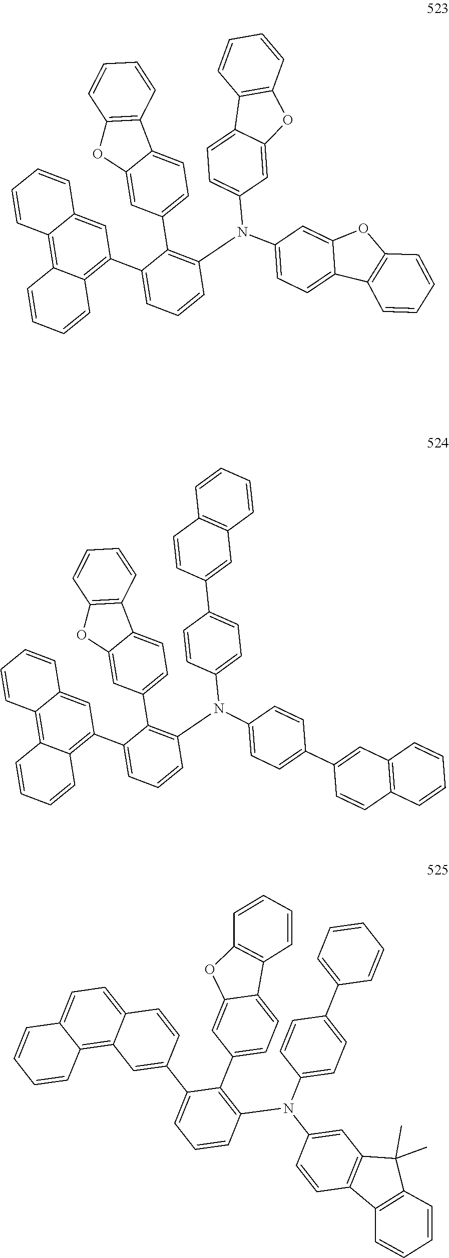 [SMILES: c1cc(-c2cc3ccccc3c3ccccc23)c(-c2ccc3c(c2)oc2ccccc23)c(N(c2ccc3c(c2)oc2ccccc23)c2ccc3c(c2)oc2ccccc23)c1 ; c1cc(-c2cc3ccccc3c3ccccc23)c(-c2ccc3c(c2)oc2ccccc23)c(N(c2ccc(-c3ccc4ccccc4c3)cc2)c2ccc(-c3ccc4ccccc4c3)cc2)c1 ; CC1(C)c2ccccc2-c2ccc(N(c3ccc(-c4ccccc4)cc3)c3cccc(-c4ccc5ccc6ccccc6c5c4)c3-c3ccc4c(c3)oc3ccccc34)cc21]

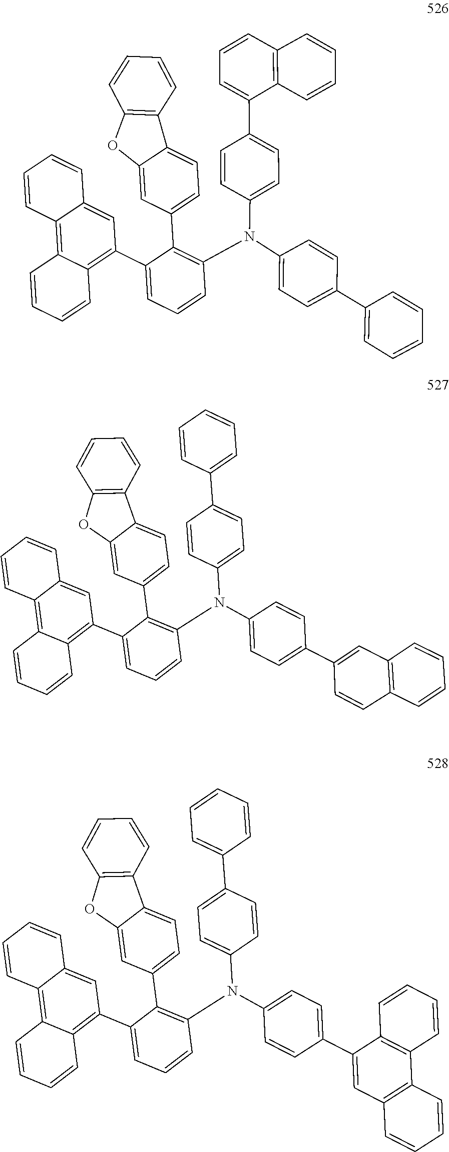 [SMILES: c1ccc(-c2ccc(N(c3ccc(-c4cccc5ccccc45)cc3)c3cccc(-c4cc5ccccc5c5ccccc45)c3-c3ccc4c(c3)oc3ccccc34)cc2)cc1 ; c1ccc(-c2ccc(N(c3ccc(-c4ccc5ccccc5c4)cc3)c3cccc(-c4cc5ccccc5c5ccccc45)c3-c3ccc4c(c3)oc3ccccc34)cc2)cc1 ; c1ccc(-c2ccc(N(c3ccc(-c4cc5ccccc5c5ccccc45)cc3)c3cccc(-c4cc5ccccc5c5ccccc45)c3-c3ccc4c(c3)oc3ccccc34)cc2)cc1]

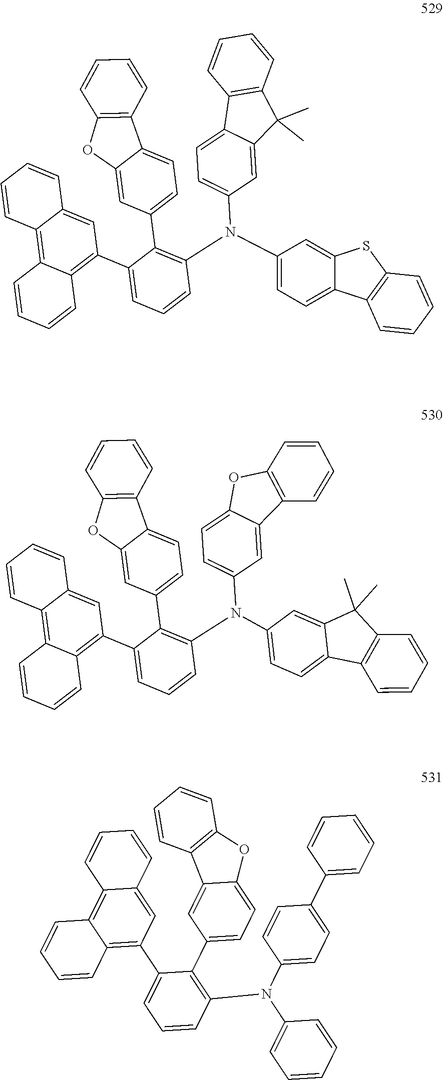 [SMILES: CC1(C)c2ccccc2-c2ccc(N(c3ccc4c(c3)sc3ccccc34)c3cccc(-c4cc5ccccc5c5ccccc45)c3-c3ccc4c(c3)oc3ccccc34)cc21 ; CC1(C)c2ccccc2-c2ccc(N(c3ccc4oc5ccccc5c4c3)c3cccc(-c4cc5ccccc5c5ccccc45)c3-c3ccc4c(c3)oc3ccccc34)cc21 ; c1ccc(-c2ccc(N(c3ccccc3)c3cccc(-c4cc5ccccc5c5ccccc45)c3-c3ccc4oc5ccccc5c4c3)cc2)cc1]

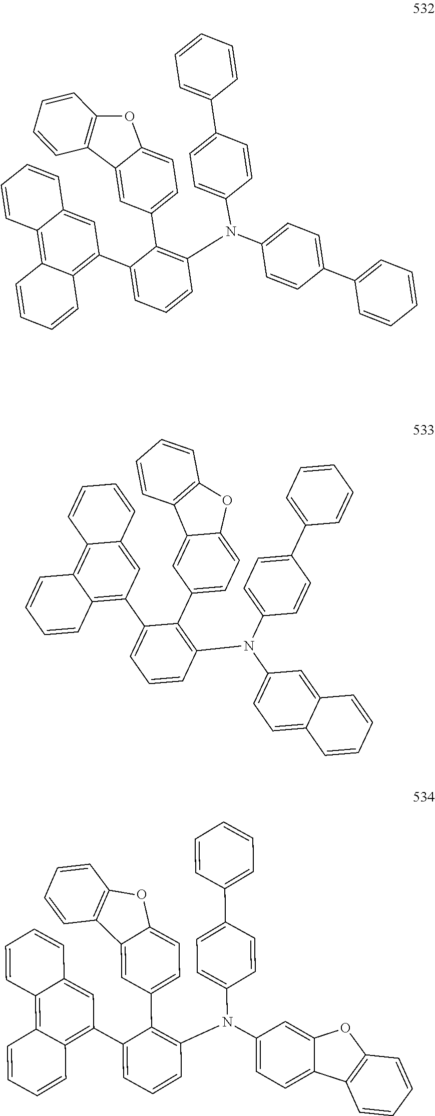 [SMILES: c1ccc(-c2ccc(N(c3ccc(-c4ccccc4)cc3)c3cccc(-c4cc5ccccc5c5ccccc45)c3-c3ccc4oc5ccccc5c4c3)cc2)cc1 ; c1ccc(-c2ccc(N(c3ccc4ccccc4c3)c3cccc(-c4cc5ccccc5c5ccccc45)c3-c3ccc4oc5ccccc5c4c3)cc2)cc1 ; c1ccc(-c2ccc(N(c3ccc4c(c3)oc3ccccc34)c3cccc(-c4cc5ccccc5c5ccccc45)c3-c3ccc4oc5ccccc5c4c3)cc2)cc1]

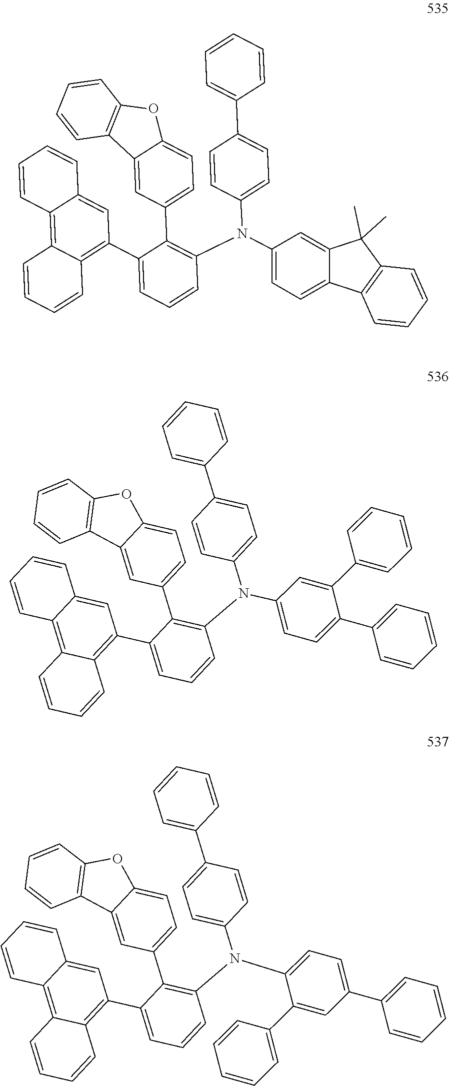 [SMILES: CC1(C)c2ccccc2-c2ccc(N(c3ccc(-c4ccccc4)cc3)c3cccc(-c4cc5ccccc5c5ccccc45)c3-c3ccc4oc5ccccc5c4c3)cc21 ; c1ccc(-c2ccc(N(c3ccc(-c4ccccc4)c(-c4ccccc4)c3)c3cccc(-c4cc5ccccc5c5ccccc45)c3-c3ccc4oc5ccccc5c4c3)cc2)cc1 ; c1ccc(-c2ccc(N(c3ccc(-c4ccccc4)cc3-c3ccccc3)c3cccc(-c4cc5ccccc5c5ccccc45)c3-c3ccc4oc5ccccc5c4c3)cc2)cc1]

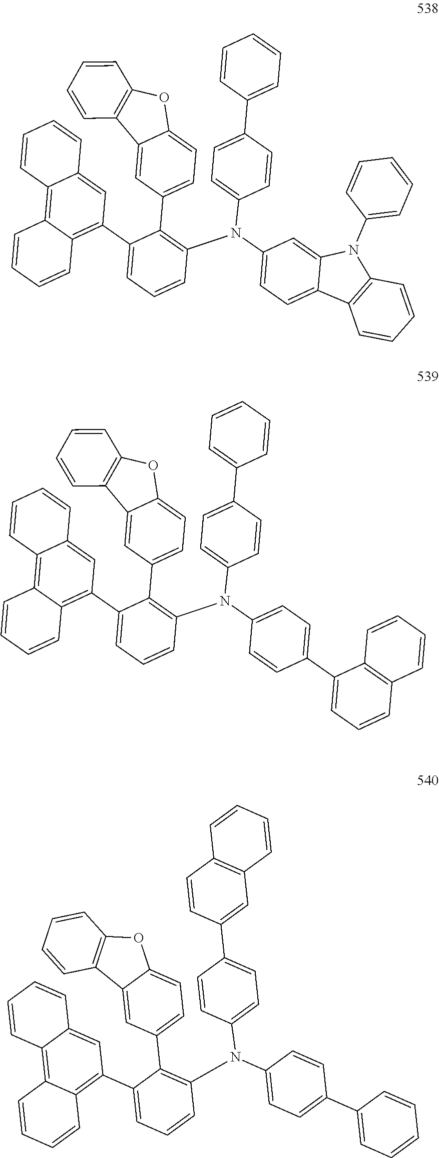 [SMILES: c1ccc(-c2ccc(N(c3ccc4c5ccccc5n(-c5ccccc5)c4c3)c3cccc(-c4cc5ccccc5c5ccccc45)c3-c3ccc4oc5ccccc5c4c3)cc2)cc1 ; c1ccc(-c2ccc(N(c3ccc(-c4cccc5ccccc45)cc3)c3cccc(-c4cc5ccccc5c5ccccc45)c3-c3ccc4oc5ccccc5c4c3)cc2)cc1 ; c1ccc(-c2ccc(N(c3ccc(-c4ccc5ccccc5c4)cc3)c3cccc(-c4cc5ccccc5c5ccccc45)c3-c3ccc4oc5ccccc5c4c3)cc2)cc1]

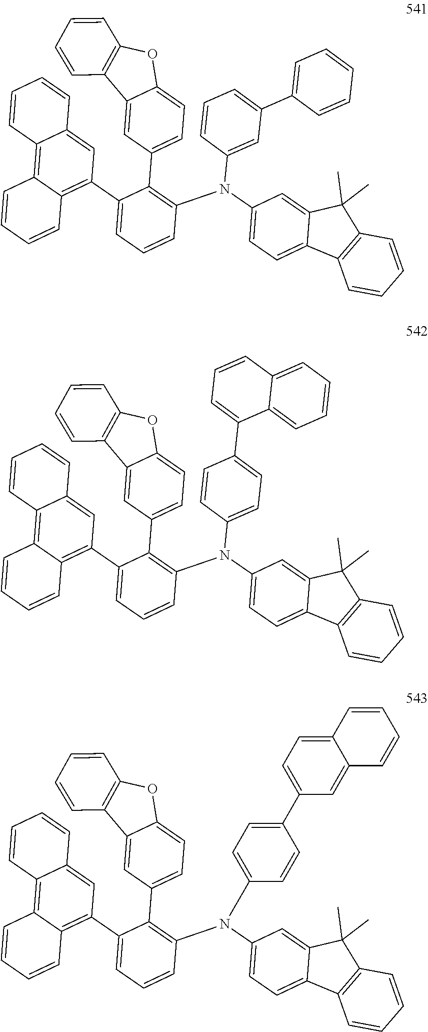

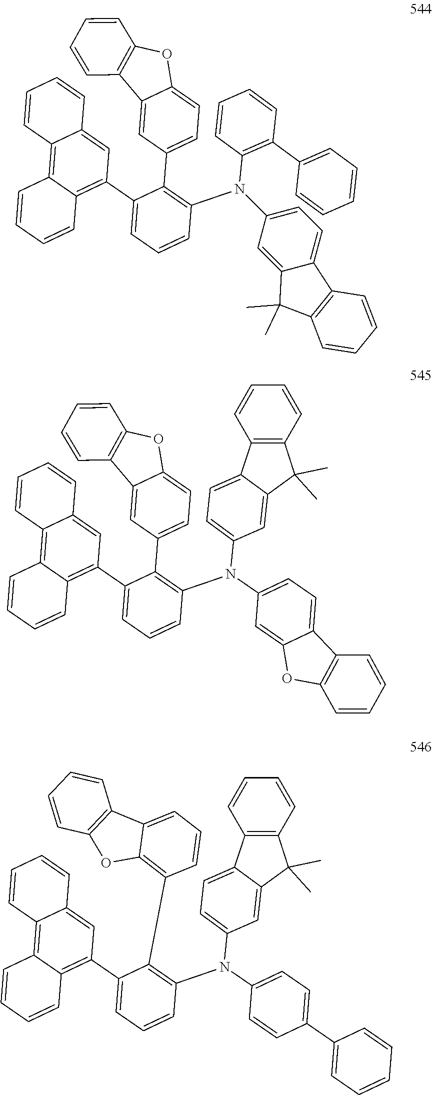 [SMILES: CC1(C)c2ccccc2-c2ccc(N(c3ccccc3-c3ccccc3)c3cccc(-c4cc5ccccc5c5ccccc45)c3-c3ccc4oc5ccccc5c4c3)cc21 ; CC1(C)c2ccccc2-c2ccc(N(c3ccc4c(c3)oc3ccccc34)c3cccc(-c4cc5ccccc5c5ccccc45)c3-c3ccc4oc5ccccc5c4c3)cc21 ; CC1(C)c2ccccc2-c2ccc(N(c3ccc(-c4ccccc4)cc3)c3cccc(-c4cc5ccccc5c5ccccc45)c3-c3cccc4c3oc3ccccc34)cc21]

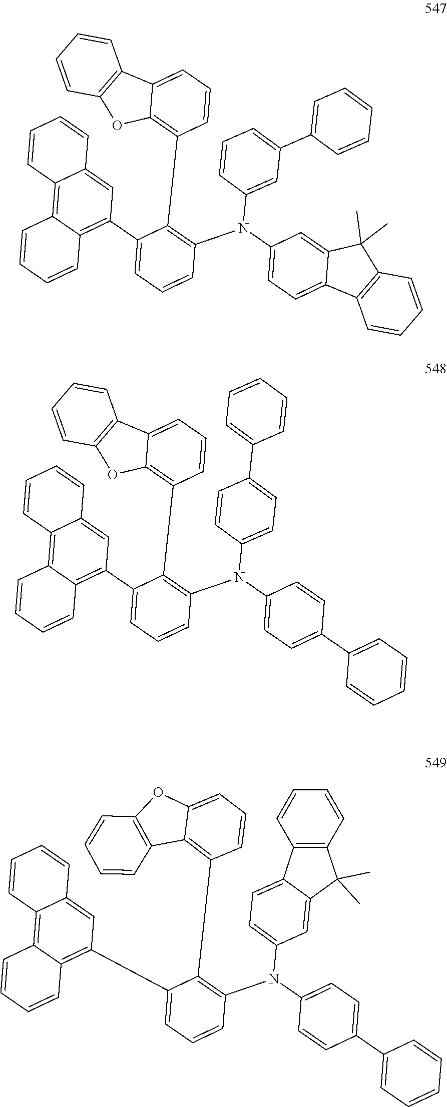 [SMILES: CC1(C)c2ccccc2-c2ccc(N(c3cccc(-c4ccccc4)c3)c3cccc(-c4cc5ccccc5c5ccccc45)c3-c3cccc4c3oc3ccccc34)cc21 ; c1ccc(-c2ccc(N(c3ccc(-c4ccccc4)cc3)c3cccc(-c4cc5ccccc5c5ccccc45)c3-c3cccc4c3oc3ccccc34)cc2)cc1 ; CC1(C)c2ccccc2-c2ccc(N(c3ccc(-c4ccccc4)cc3)c3cccc(-c4cc5ccccc5c5ccccc45)c3-c3cccc4oc5ccccc5c34)cc21]

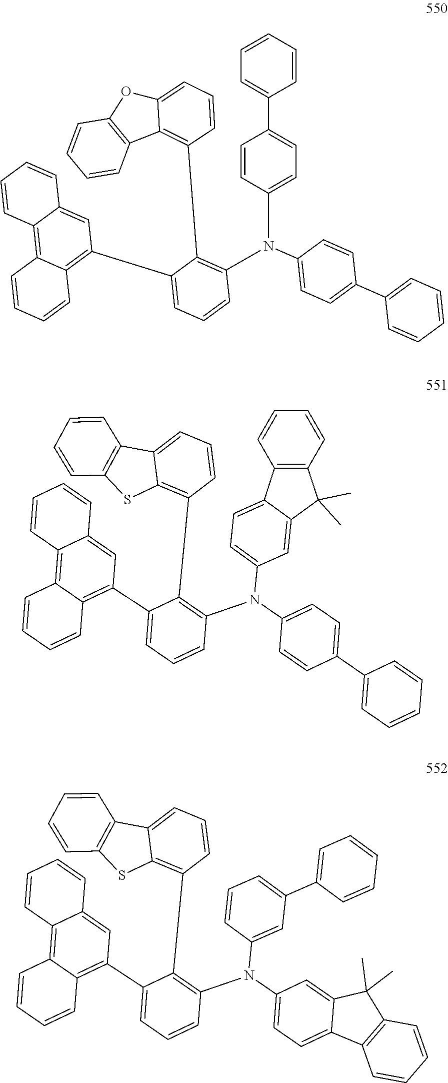 [SMILES: c1ccc(-c2ccc(N(c3ccc(-c4ccccc4)cc3)c3cccc(-c4cc5ccccc5c5ccccc45)c3-c3cccc4oc5ccccc5c34)cc2)cc1 ; CC1(C)c2ccccc2-c2ccc(N(c3ccc(-c4ccccc4)cc3)c3cccc(-c4cc5ccccc5c5ccccc45)c3-c3cccc4c3sc3ccccc34)cc21 ; CC1(C)c2ccccc2-c2ccc(N(c3cccc(-c4ccccc4)c3)c3cccc(-c4cc5ccccc5c5ccccc45)c3-c3cccc4c3sc3ccccc34)cc21]

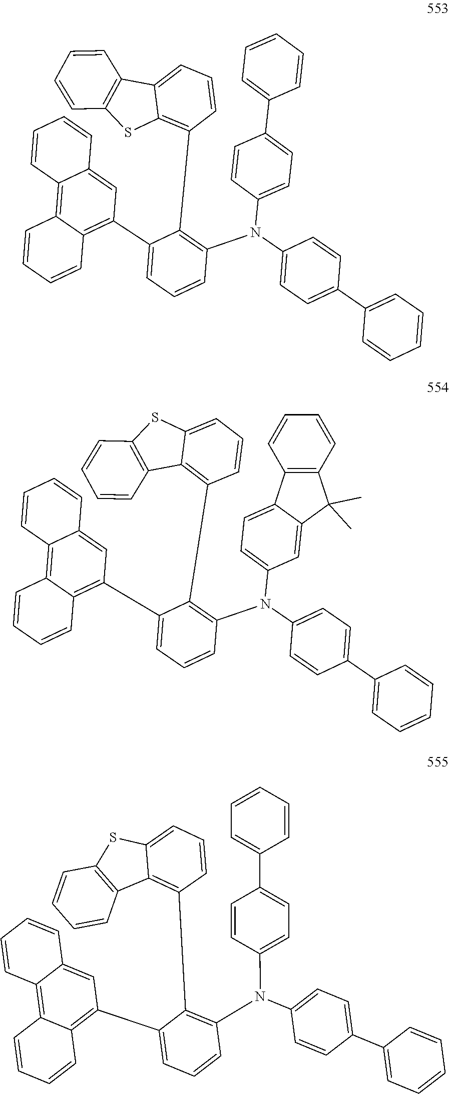 [SMILES: c1ccc(-c2ccc(N(c3ccc(-c4ccccc4)cc3)c3cccc(-c4cc5ccccc5c5ccccc45)c3-c3cccc4c3sc3ccccc34)cc2)cc1 ; CC1(C)c2ccccc2-c2ccc(N(c3ccc(-c4ccccc4)cc3)c3cccc(-c4cc5ccccc5c5ccccc45)c3-c3cccc4sc5ccccc5c34)cc21 ; c1ccc(-c2ccc(N(c3ccc(-c4ccccc4)cc3)c3cccc(-c4cc5ccccc5c5ccccc45)c3-c3cccc4sc5ccccc5c34)cc2)cc1]

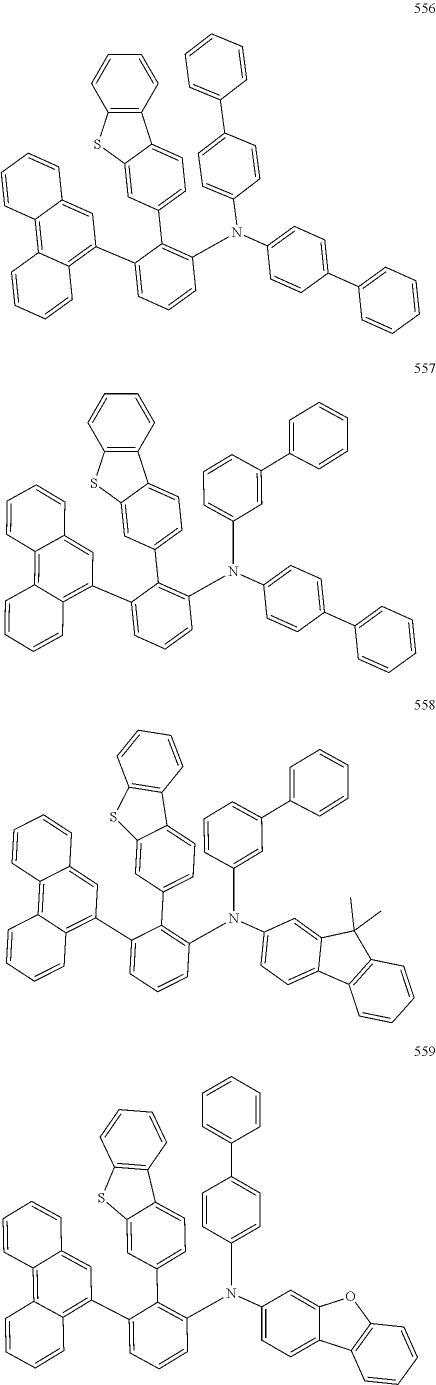 [SMILES: c1ccc(-c2ccc(N(c3ccc(-c4ccccc4)cc3)c3cccc(-c4cc5ccccc5c5ccccc45)c3-c3ccc4c(c3)sc3ccccc34)cc2)cc1 ; c1ccc(-c2ccc(N(c3cccc(-c4ccccc4)c3)c3cccc(-c4cc5ccccc5c5ccccc45)c3-c3ccc4c(c3)sc3ccccc34)cc2)cc1 ; CC1(C)c2ccccc2-c2ccc(N(c3cccc(-c4ccccc4)c3)c3cccc(-c4cc5ccccc5c5ccccc45)c3-c3ccc4c(c3)sc3ccccc34)cc21 ; c1ccc(-c2ccc(N(c3ccc4c(c3)oc3ccccc34)c3cccc(-c4cc5ccccc5c5ccccc45)c3-c3ccc4c(c3)sc3ccccc34)cc2)cc1]

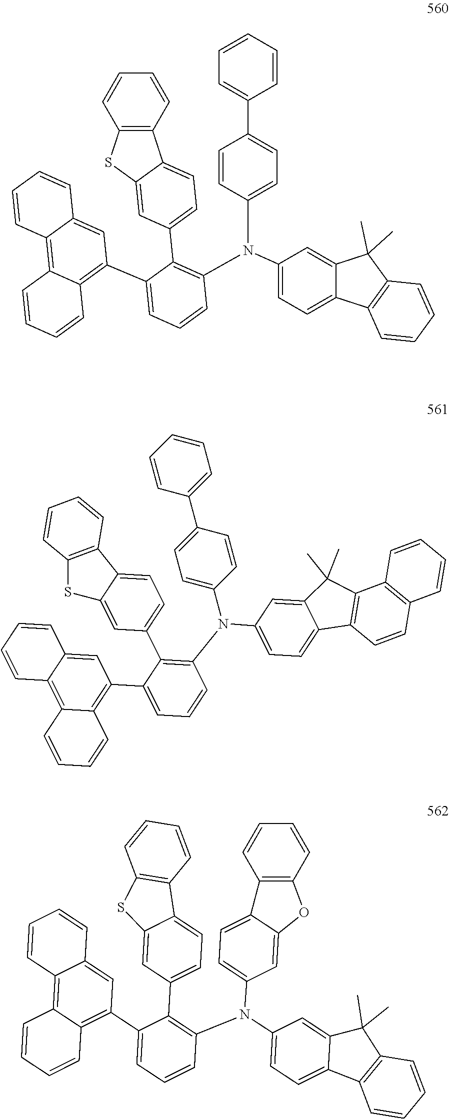 [SMILES: CC1(C)c2ccccc2-c2ccc(N(c3ccc(-c4ccccc4)cc3)c3cccc(-c4cc5ccccc5c5ccccc45)c3-c3ccc4c(c3)sc3ccccc34)cc21 ; CC1(C)c2cc(N(c3ccc(-c4ccccc4)cc3)c3cccc(-c4cc5ccccc5c5ccccc45)c3-c3ccc4c(c3)sc3ccccc34)ccc2-c2ccc3ccccc3c21 ; CC1(C)c2ccccc2-c2ccc(N(c3ccc4c(c3)oc3ccccc34)c3cccc(-c4cc5ccccc5c5ccccc45)c3-c3ccc4c(c3)sc3ccccc34)cc21]

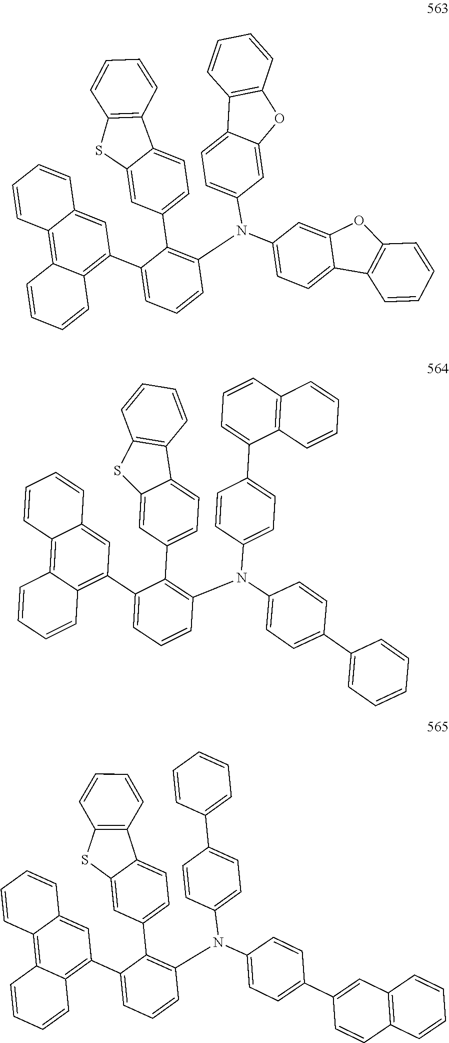 [SMILES: c1cc(-c2cc3ccccc3c3ccccc23)c(-c2ccc3c(c2)sc2ccccc23)c(N(c2ccc3c(c2)oc2ccccc23)c2ccc3c(c2)oc2ccccc23)c1 ; c1ccc(-c2ccc(N(c3ccc(-c4cccc5ccccc45)cc3)c3cccc(-c4cc5ccccc5c5ccccc45)c3-c3ccc4c(c3)sc3ccccc34)cc2)cc1 ; c1ccc(-c2ccc(N(c3ccc(-c4ccc5ccccc5c4)cc3)c3cccc(-c4cc5ccccc5c5ccccc45)c3-c3ccc4c(c3)sc3ccccc34)cc2)cc1]

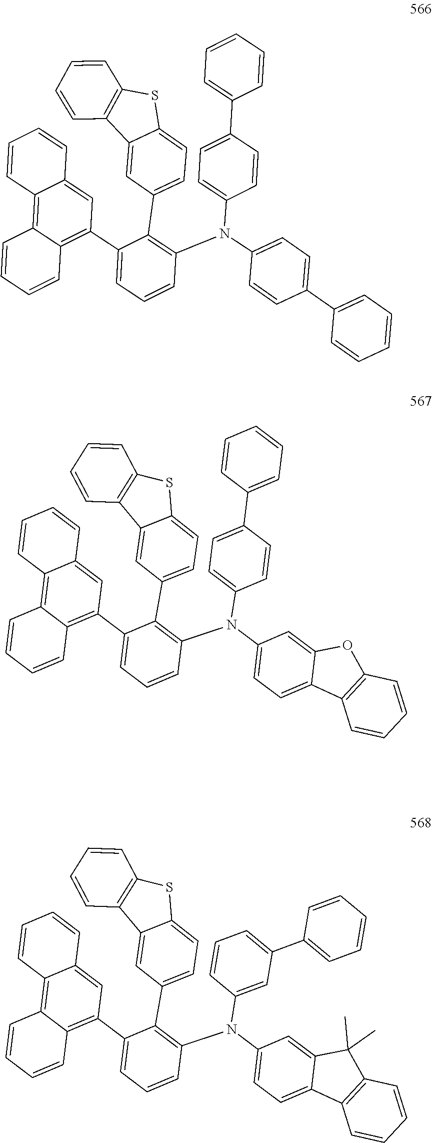 [SMILES: c1ccc(-c2ccc(N(c3ccc(-c4ccccc4)cc3)c3cccc(-c4cc5ccccc5c5ccccc45)c3-c3ccc4sc5ccccc5c4c3)cc2)cc1 ; c1ccc(-c2ccc(N(c3ccc4c(c3)oc3ccccc34)c3cccc(-c4cc5ccccc5c5ccccc45)c3-c3ccc4sc5ccccc5c4c3)cc2)cc1 ; CC1(C)c2ccccc2-c2ccc(N(c3cccc(-c4ccccc4)c3)c3cccc(-c4cc5ccccc5c5ccccc45)c3-c3ccc4sc5ccccc5c4c3)cc21]

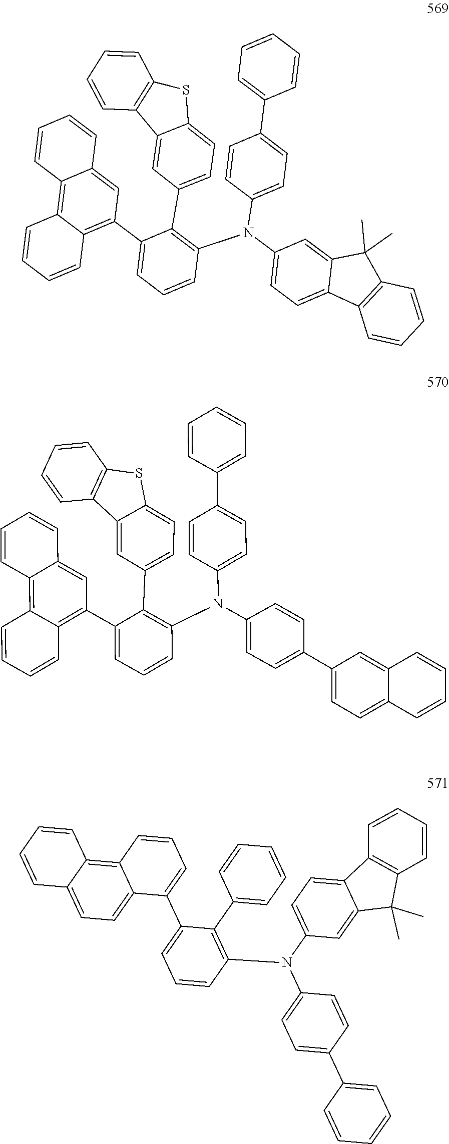 [SMILES: CC1(C)c2ccccc2-c2ccc(N(c3ccc(-c4ccccc4)cc3)c3cccc(-c4cc5ccccc5c5ccccc45)c3-c3ccc4sc5ccccc5c4c3)cc21 ; c1ccc(-c2ccc(N(c3ccc(-c4ccc5ccccc5c4)cc3)c3cccc(-c4cc5ccccc5c5ccccc45)c3-c3ccc4sc5ccccc5c4c3)cc2)cc1 ; CC1(C)c2ccccc2-c2ccc(N(c3ccc(-c4ccccc4)cc3)c3cccc(-c4cccc5c4ccc4ccccc45)c3-c3ccccc3)cc21]

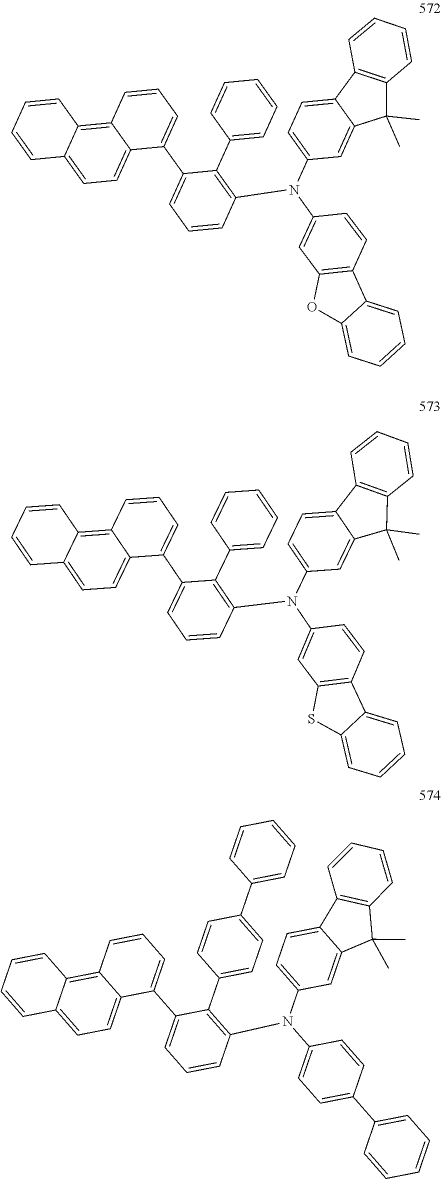 [SMILES: CC1(C)c2ccccc2-c2ccc(N(c3ccc4c(c3)oc3ccccc34)c3cccc(-c4cccc5c4ccc4ccccc45)c3-c3ccccc3)cc21 ; CC1(C)c2ccccc2-c2ccc(N(c3ccc4c(c3)sc3ccccc34)c3cccc(-c4cccc5c4ccc4ccccc45)c3-c3ccccc3)cc21 ; CC1(C)c2ccccc2-c2ccc(N(c3ccc(-c4ccccc4)cc3)c3cccc(-c4cccc5c4ccc4ccccc45)c3-c3ccc(-c4ccccc4)cc3)cc21]

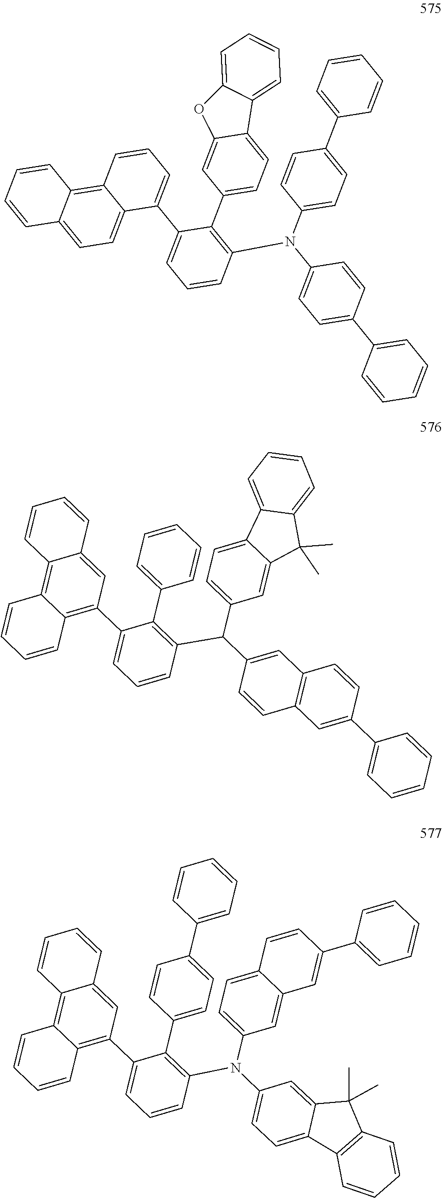

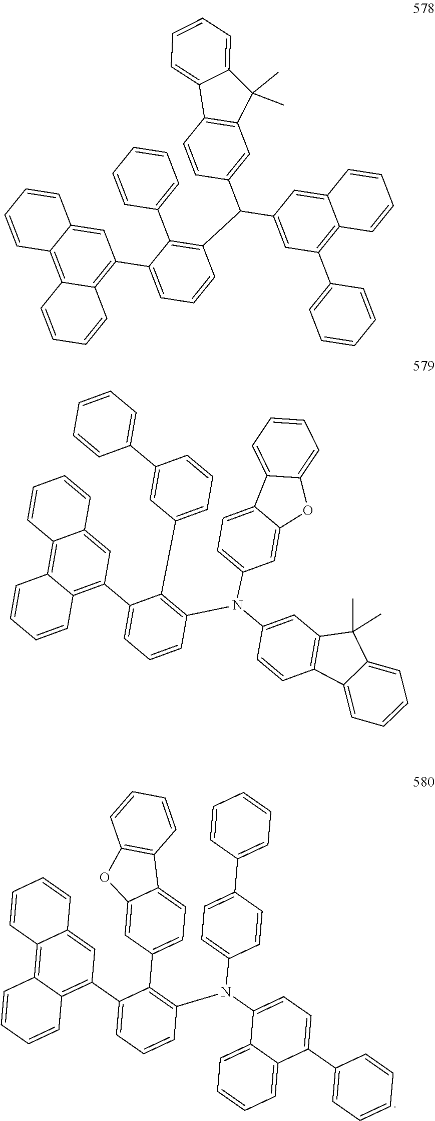

In a second aspect, the present application provides an electronic element, comprising an anode and a cathode disposed opposite to each other, and a functional layer disposed between the anode and the cathode; the functional layer comprises the organic compound of the present application.

Optionally, the functional layer comprises a second hole transport layer, which comprises the organic compound of the present application.

Optionally, the electronic element is an organic electroluminescent device or a photoelectric conversion device.

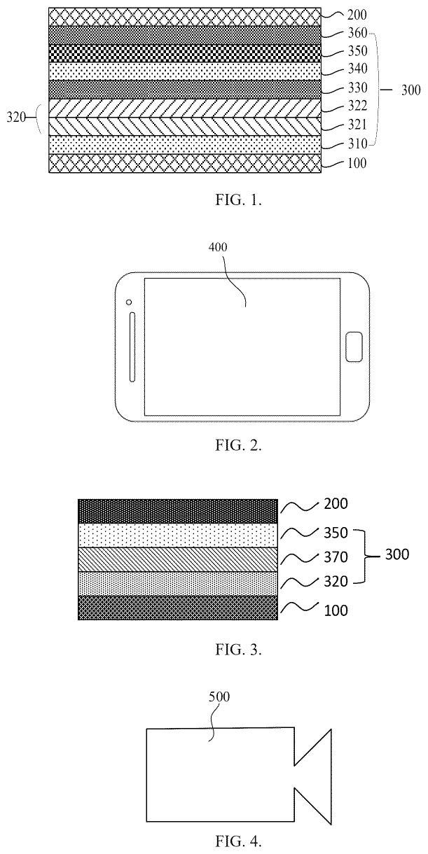

In one embodiment, the electronic element is an organic electroluminescent device. As shown in FIG. 1, the organic electroluminescent device may comprise an anode 100, a hole transport layer 320, an organic light-emitting layer 330, a hole blocking layer 340, an electron transport layer 350, and a cathode 200 that are stacked sequentially.

In one specific embodiment, the organic electroluminescent device is a red organic electroluminescent device.

Optionally, the anode 100 comprises the following anode materials, which are optionally a high work function material contributing to injection of holes into the functional layer. Specific examples of the anode material include: metals such as nickel, platinum, vanadium, chromium, copper, zinc and gold, or alloys thereof; metal oxides such as zinc oxide, indium oxide, indium tin oxide (ITO), and indium zinc oxide (IZO); combinations of metals and oxides, such as ZnO:Al or SnO₂:Sb; or conductive polymers such as poly(3-methylthiophene), poly[3,4-(ethylene-1,2-dioxy)thiophene](PEDT), polypyrrole, and polyaniline, but are not limited thereto. Preferably, a transparent electrode comprising indium tin oxide (ITO) as the anode is included.

Optionally, the hole transport layer 320 comprises the organic compound of the present application.

Optionally, the hole transport layer 320 comprises a first hole transport layer 321 and a second hole transport layer 322 arranged in stack, and the first hole transport layer 321 is closer to the anode than the second hole transport layer 322.

Optionally, the first hole transport layer 321 comprises one or more hole transport materials. The hole transport materials may be selected from carbazole polymers, carbazole-linked triarylamine based compounds, and other types of compounds. Those skilled in the art may make a selection with reference to the prior art. For example, the material of the first hole transport layer 321 is selected from the group consisting of the following compounds:

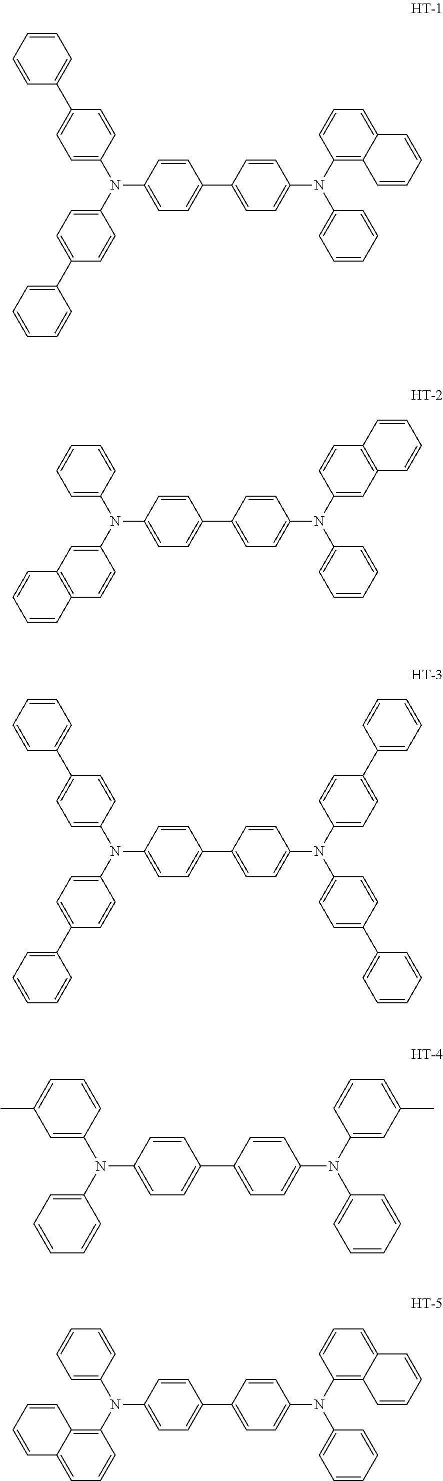

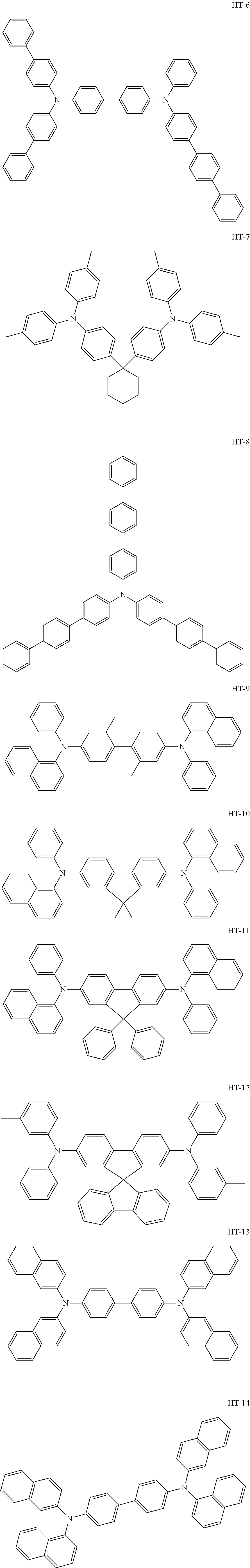

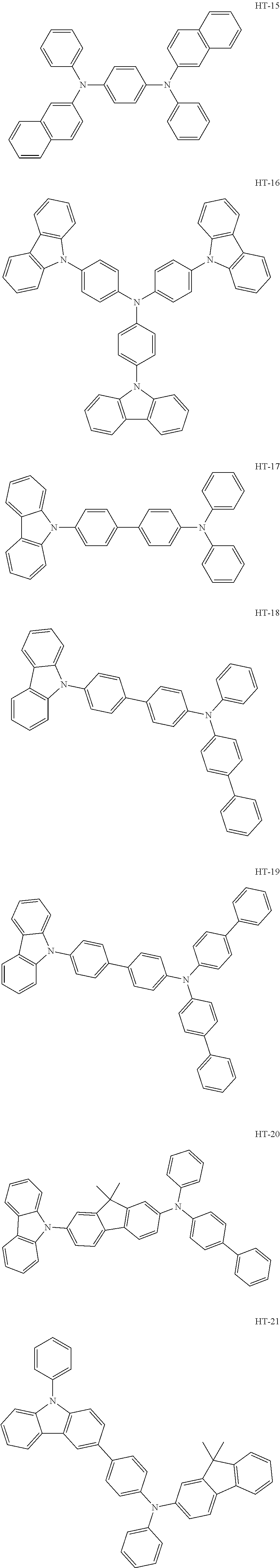

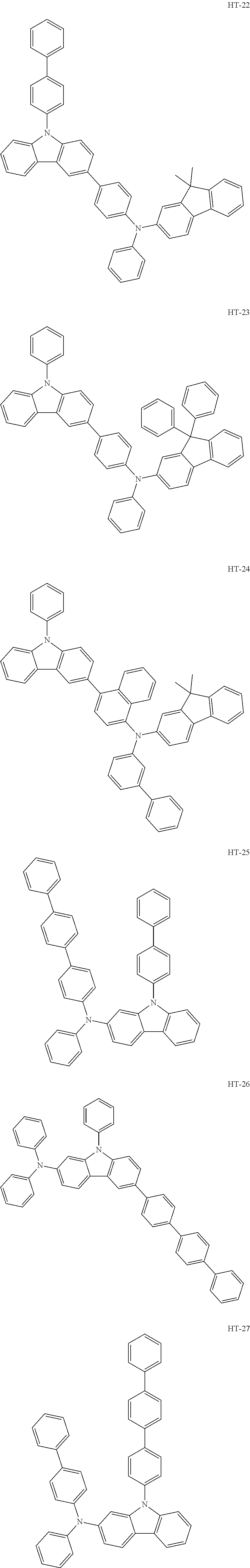

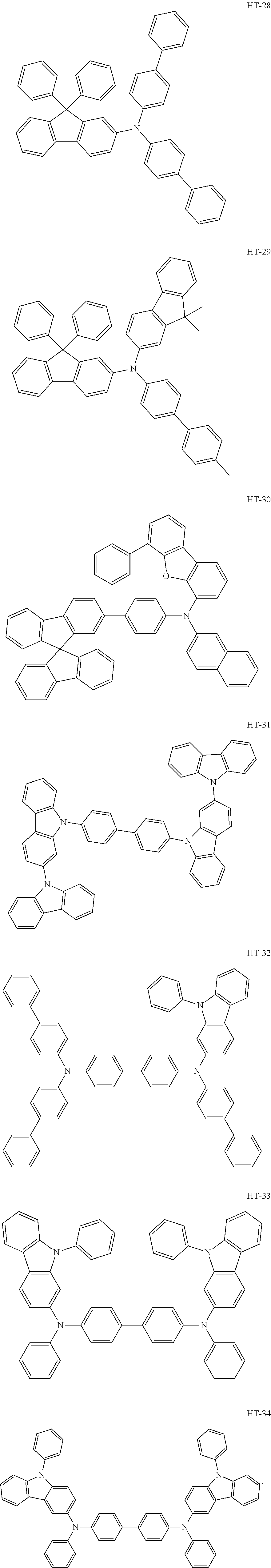

In one specific embodiment, the first hole transport layer 321 is a Compound HT-1.

Optionally, the second hole transport layer 322 comprises the organic compound of the present application.

Optionally, the organic light-emitting layer 330 may be composed of a single luminescent layer material or may comprise a host material and a doping material. Optionally, the organic light-emitting layer 330 is composed of a host material and a doping material. The holes injected into the organic light-emitting layer 330 and the electrons injected into the organic light-emitting layer 330 can recombine in the organic light-emitting layer 330 to form excitons. The excitons transmit energy to the host material, and the host material transmits the energy to the doping material, thereby enabling the doping material to emit light.

The host material of the organic light-emitting layer 330 may be a metal chelating compound, a stilbene-based derivative, an aromatic amine derivative, a dibenzofuran derivative, or other types of materials. It is not particularly limited in the present application. The host material may be a single host material, or a mixed host material.

In one embodiment of the present application, the host material of the organic light-emitting layer 330 is RH-1

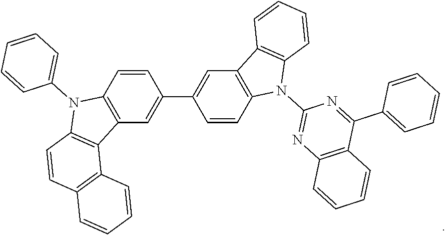 [SMILES: c1ccc(-c2nc(-n3c4ccccc4c4cc(-c5ccc6c(c5)c5c7ccccc7ccc5n6-c5ccccc5)ccc43)nc3ccccc23)cc1]

The doping material of the organic light-emitting layer 330 can be selected with reference to the prior art, and for example, may be selected from an iridium (III) organometallic complex, a platinum (II) organometallic complex, and a ruthenium (II) complex, etc. The specific examples of the doping material include but are not limited to,

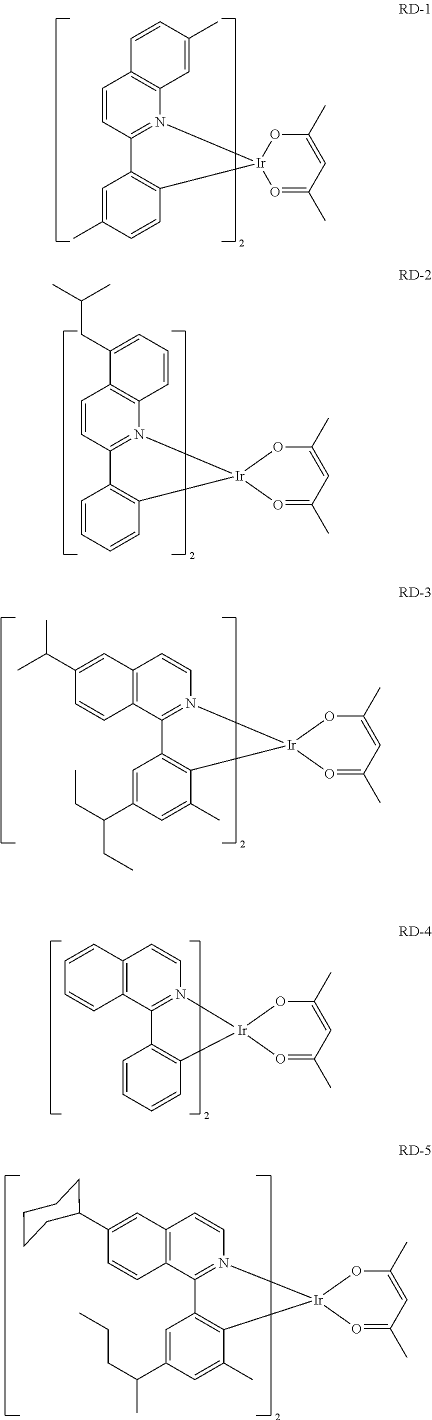

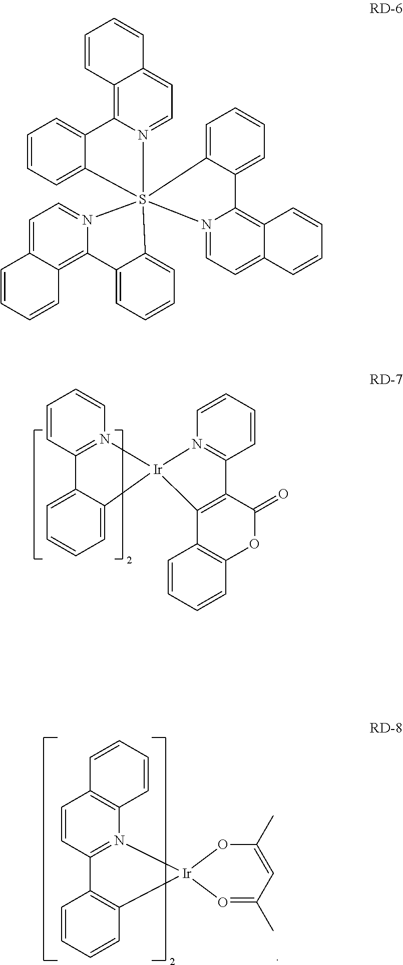

In one embodiment of the present application, the doping material of the organic light-emitting layer 340 is RD-1.

Optionally, the hole blocking layer 340 comprises one or more hole blocking material(s), which may be selected from carbazole polymers or other types of compounds. It is not particularly limited in the present application. In some embodiments of the present application, the hole blocking layer 340 is HB-1.

Optionally, the electron transport layer 350 may be either a single-layer structure or a multi-layer structure and may comprise one or more electron transport material(s). The electron transport material may typically comprise a metal complex or a nitrogen-containing heterocyclic derivative, in which the metal complex material may be, for example, selected from LiQ, Alq₃, and Bepq₂, etc; the nitrogen-containing heterocyclic derivative may be an aromatic ring compound with a nitrogen-containing 5-membered or 6-membered skeleton, a fused aromatic ring compound with a nitrogen-containing 5-membered or 6-membered skeleton, etc. Specific examples include but are not limited to 1,10-phenanthroline based compounds such as ET-1, Bphen, Nbphen, DBimiBphen, and BimiBphen, and anthracene-based compounds, triazine based compounds, or pyrimidine-based compounds with nitrogen-containing heteroaryl as shown below. In one embodiment of the present application, the electron transport layer 350 is composed of ET-1 and LiQ.

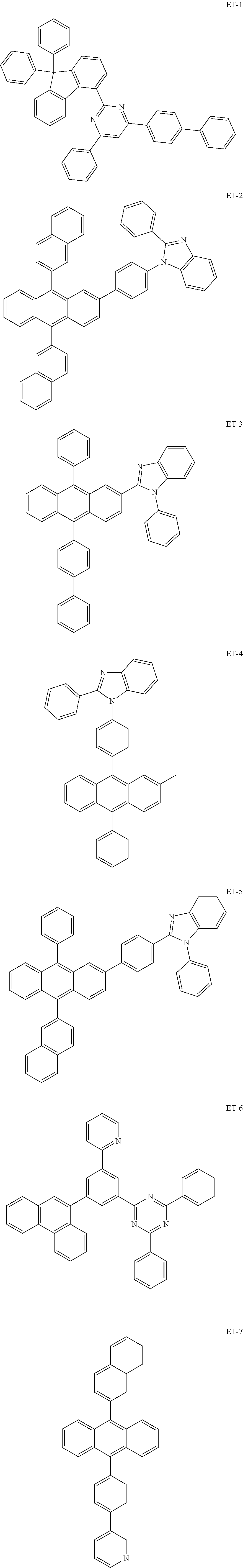

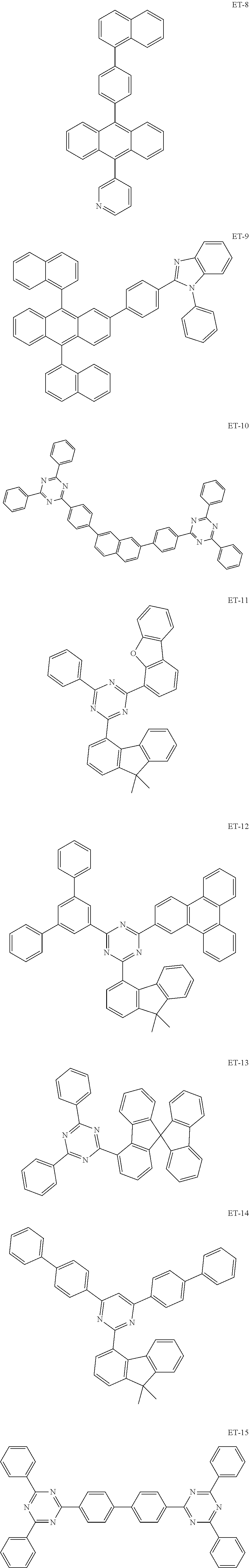

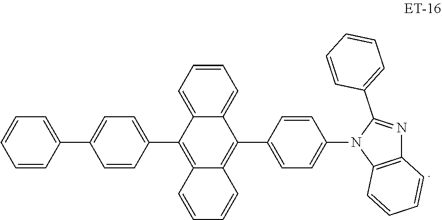 [SMILES: c1ccc(-c2ccc(-c3c4ccccc4c(-c4ccc(-n5c(-c6ccccc6)nc6ccccc65)cc4)c4ccccc34)cc2)cc1]

In the present application, the cathode 200 may comprise a cathode material, which is a low work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, and lead, or alloys thereof; or multilayer materials such as LiF/Al, Liq/Al, LiO₂/Al, LiF/Ca, LiF/Al, and BaF₂/Ca. Preferably, a metal electrode comprising magnesium and silver as the cathode is included.

Optionally, as shown in FIG. 1, a hole injection layer 310 may be further provided between the anode 100 and the first hole transport layer 321 to enhance the ability to inject holes into the first hole transport layer 321. The hole injection layer 310 may choose to use a benzidine derivative, a starburst arylamine-based compound, a phthalocyanine derivative or other materials. It is not particularly limited in the present application. For example, the compound contained in the hole injection layer 310 is selected from the group consisting of the following compounds:

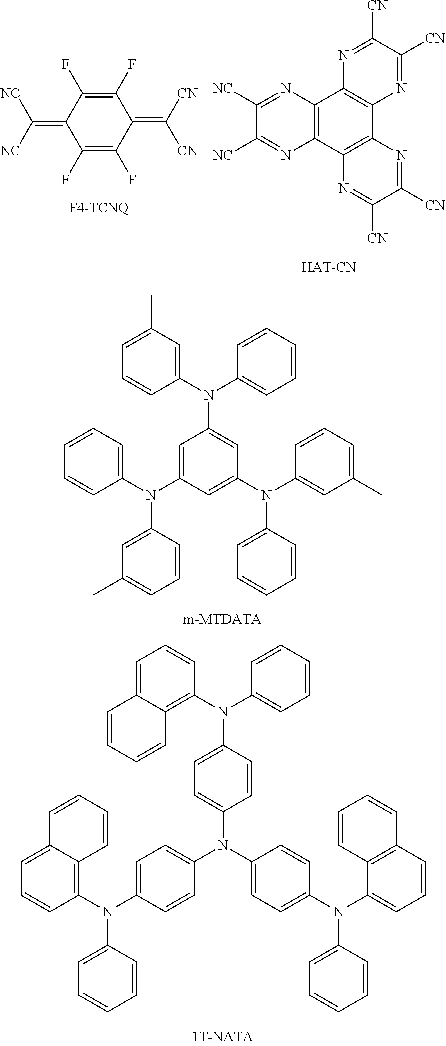

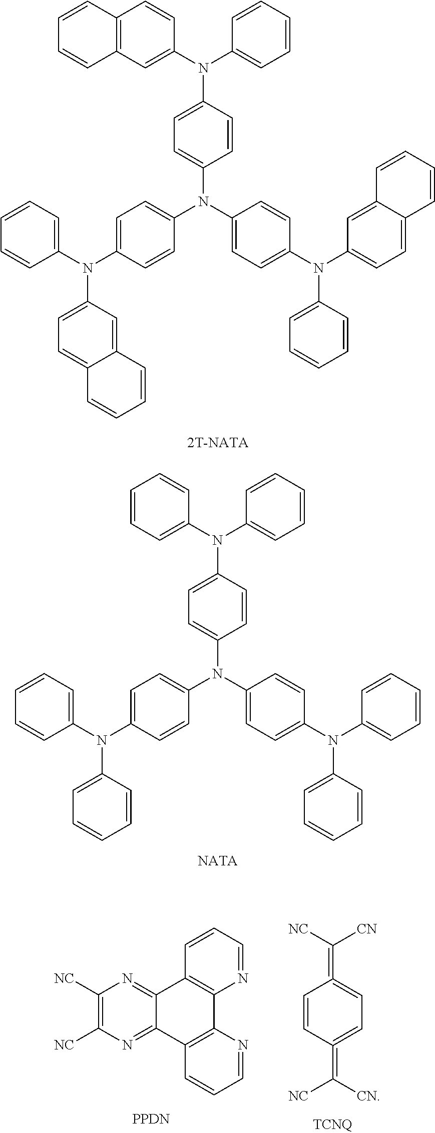

In one specific embodiment of the present application, the hole injection layer 310 is HAT-CN.

Optionally, as shown in FIG. 1, an electron injection layer 360 is further provided between the cathode 200 and the electron transport layer 350 to enhance the ability to inject electrons into the electron transport layer 350. The electron injection layer 360 may comprise an inorganic material such as an alkali metal sulfide and an alkali metal halide or may comprise a complex of an alkali metal and an organic compound. In one specific embodiment of the present application, the electron injection layer 360 comprises is Yb.

According to another embodiment, the electronic component is a photoelectric conversion device. As shown in FIG. 3, the photoelectric conversion device may include an anode 100 and a cathode 200 disposed opposite to each other, and a functional layer 300 disposed between the anode 100 and the cathode 200; the functional layer 300 comprises the organic compound provided in the present application.

According to a specific embodiment, as shown in FIG. 3, the photoelectric conversion device comprises an anode 100, a hole transport layer 320, a photoelectric conversion layer 370, an electron transport layer 350, and a cathode 200 stacked in sequence. Optionally, the hole transport layer 320 comprises the organic compound of the present application.

Optionally, the photoelectric conversion device is a solar cell, and in particular an organic thin film solar cell. For example, in one embodiment of the present application, the solar cell comprises an anode, a hole transport layer, a photoelectric conversion layer, an electron transport layer, and a cathode sequentially stacked, wherein the hole transport layer comprises the organic compound of the present application.

In a third aspect, the present application provides an electronic apparatus, comprising the electronic element described in the second aspect of the present application.

According to one embodiment, as shown in FIG. 2, the electronic apparatus is a first electronic apparatus 400 comprising the above-described organic electroluminescent device. The first electronic apparatus 400 may be a display apparatus, a lighting apparatus, an optical communication apparatus, or other type of electronic apparatus, examples of which may include, but are not limited to, a computer screen, a mobile phone screen, a television, an electronic paper, an emergency lamp, and an optical module, etc.

According to another embodiment, as shown in FIG. 4, the electronic apparatus is a second electronic apparatus 500 comprising the above-described organic electroluminescent device. The second electronic apparatus 500 can be, for example, a solar power generation equipment, a photodetector, a fingerprint recognition equipment, an optical module, a CCD camera, and other types of electronic apparatus.

The synthesis method of the organic compound in the present application will be demonstrated in detail with the following synthesis examples, but the present application is not limited in any way by this.

Compounds for which synthesis methods are not mentioned in the present application may be obtained through commercial sources.

### Synthesis Example

### 1. Synthesis of IM-1

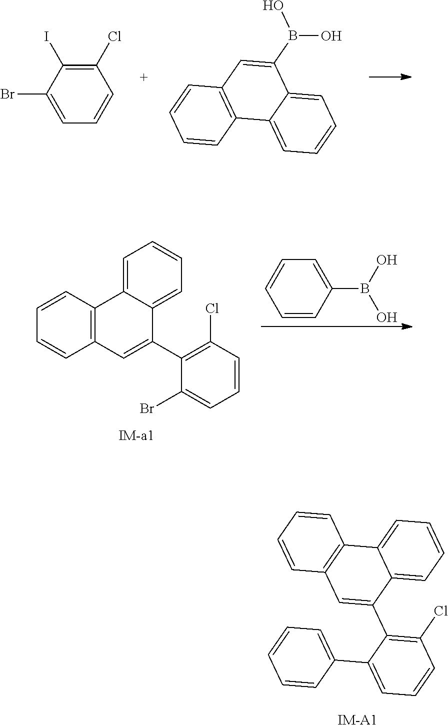

(1) Under a nitrogen atmosphere, 1-bromo-3-chloro-2-iodobenzene (120.0 g, 378.13 mmol), 9-phenanthreneboronic acid (83.8 g, 377.38 mmol), potassium carbonate (114.9 g, 831.87 mmol), tetrakis(triphenylphosphine)palladium (8.74 g, 7.56 mmol), tetrabutylammonium bromide (TBAB, 24.4 g, 75.62 mmol), toluene (1.2 L), ethanol (600 mL), and water (240 mL) were added to a three necked flask. The resulting mixture was stirred and heated to 70° C. to 75° C., followed by refluxing for 12 hours of reaction. After the reaction was completed, the reaction solution was cooled to the room temperature, and was extracted with dichloromethane. After separation, the organic phase was washed with water until neutral, followed by drying, filtering, and concentrating, to yield a crude product. The crude product was recrystallized from ethyl acetate and n-hexane to LC >98%. After drying, a IM-a1 as a white solid was obtained (101.9 g, yield 73.4%).
(2) Under a nitrogen atmosphere, IM-a1 (20.0 g, 54.40 mmol), phenylboronic acid (6.64 g, 54.50 mmol), potassium carbonate (16.5 g, 119.50 mmol), tetrakis(triphenylphosphine) palladium (1.3 g, 1.14 mmol), tetrabutylammonium bromide (TBAB, 3.5 g, 10.88 mmol), toluene (200 mL), ethanol (100 mL), and water (40 mL) were added to a reaction flask. The resulting mixture was stirred and heated to 70° C. to 75° C., followed by refluxing for 24 hours of reaction. After the reaction was completed, the reaction solution was cooled to the room temperature, and was extracted with dichloromethane. After separation, the organic phase was washed with water until neutral, followed by drying, filtering, and concentrating, to yield a crude product. The crude product was recrystallized from dichloromethane and n-hexane to LC >98%, and after drying, IM-A1 as a white solid was obtained (15.0 g, yield 75.6%).

The intermediates listed in Table 1 were prepared using the same method as for IM-a1, except that Raw material 1 was used instead of 1-bromo-3-chloro-2-iodobenzene, and Raw material 2 was used instead of 9-phenanthreneboronic acid. The main raw materials utilized, the intermediates synthesized, and yields thereof are shown in Table 1.

TABLE 1

| Raw material 1 | Raw material 2 | Intermediate | Yield/% |
|---|---|---|---|
| 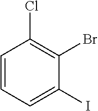 | 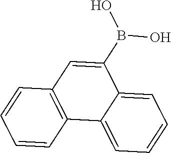 [SMILES: OB(O)c1cc2ccccc2c2ccccc12] | 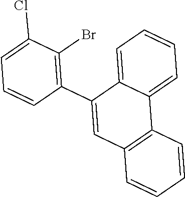 [SMILES: Clc1cccc(-c2cc3ccccc3c3ccccc23)c1Br] IM-b1 | 72.8 |
| 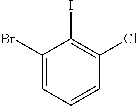 | 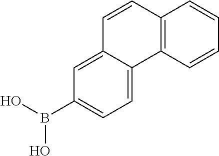 [SMILES: OB(O)c1ccc2c(ccc3ccccc32)c1] | 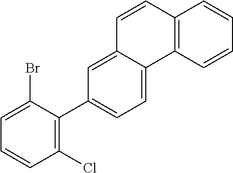 [SMILES: Clc1cccc(Br)c1-c1ccc2c(ccc3ccccc32)c1] IM-a2 | 73.5 |
| 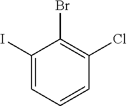 | 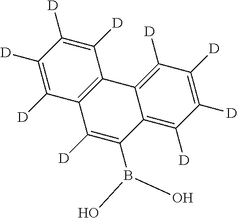 [SMILES: [2H]c1c([2H])c([2H])c2c(c1[2H])c([2H])c(B(O)O)c1c([2H])c([2H])c([2H])c([2H])c12] | 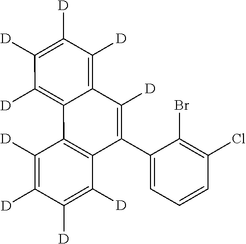 [SMILES: [2H]c1c([2H])c([2H])c2c(c1[2H])c([2H])c(-c1cccc(Cl)c1Br)c1c([2H])c([2H])c([2H])c([2H])c12] IM-b2 | 65.1 |
| 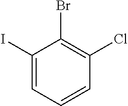 | 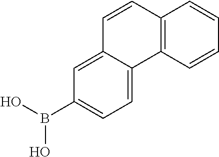 [SMILES: OB(O)c1ccc2c(ccc3ccccc32)c1] | 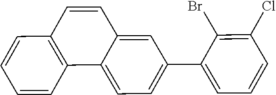 [SMILES: Clc1cccc(-c2ccc3c(ccc4ccccc43)c2)c1Br] IM-b3 | 72.8 |
| 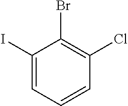 | 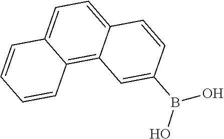 [SMILES: OB(O)c1ccc2ccc3ccccc3c2c1] | 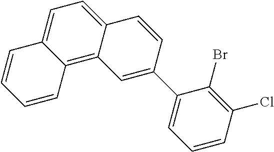 [SMILES: Clc1cccc(-c2ccc3ccc4ccccc4c3c2)c1Br] IM-b4 | 68.7 |
| 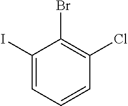 | 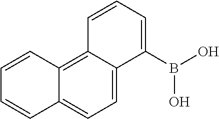 [SMILES: OB(O)c1cccc2c1ccc1ccccc12] | 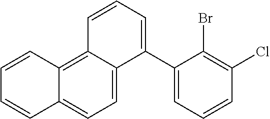 [SMILES: Clc1cccc(-c2cccc3c2ccc2ccccc23)c1Br] IM-b5 | 62.4 |

The intermediates listed in Table 2 were prepared using the same method as for IM-A1, except that Raw material 3 was used instead of IM-a1 and Raw material 4 was used instead of phenylboronic acid. The main raw materials utilized, the intermediates synthesized, and yields thereof are shown in Table 2.

TABLE 2

| | | | Yield/ |
|---|---|---|---|
| Raw material 3 | Raw material 4 | Intermediate | % |
| 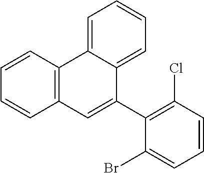 [SMILES: Clc1cccc(Br)c1-c1cc2ccccc2c2ccccc12] IM-a1 | 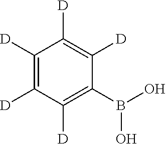 [SMILES: [2H]c1c([2H])c([2H])c(B(O)O)c([2H])c1[2H]] | 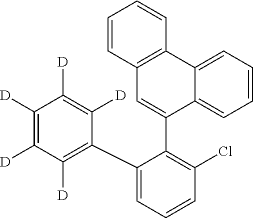 [SMILES: [2H]c1c([2H])c([2H])c(-c2cccc(Cl)c2-c2cc3ccccc3c3ccccc23)c([2H])c1[2H]] IM-A2 | 68.9 |
| 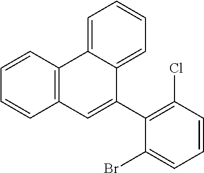 [SMILES: Clc1cccc(Br)c1-c1cc2ccccc2c2ccccc12] IM-a1 | 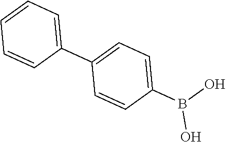 [SMILES: OB(O)c1ccc(-c2ccccc2)cc1] | 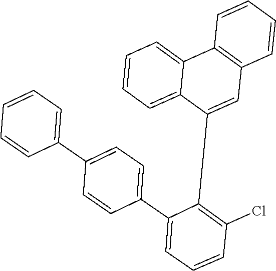 [SMILES: Clc1cccc(-c2ccc(-c3ccccc3)cc2)c1-c1cc2ccccc2c2ccccc12] IM-A3 | 70.4 |
| 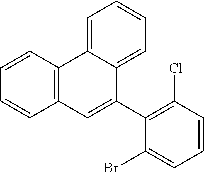 [SMILES: Clc1cccc(Br)c1-c1cc2ccccc2c2ccccc12] IM-a1 | 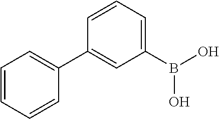 [SMILES: OB(O)c1cccc(-c2ccccc2)c1] | 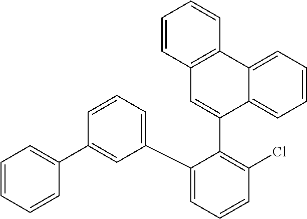 [SMILES: Clc1cccc(-c2cccc(-c3ccccc3)c2)c1-c1cc2ccccc2c2ccccc12] IM-A4 | 64.2 |
| 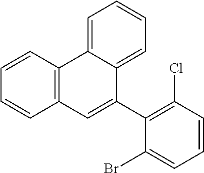 [SMILES: Clc1cccc(Br)c1-c1cc2ccccc2c2ccccc12] IM-a1 | 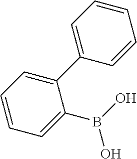 [SMILES: OB(O)c1ccccc1-c1ccccc1] | 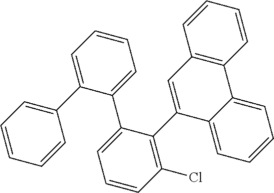 [SMILES: Clc1cccc(-c2ccccc2-c2ccccc2)c1-c1cc2ccccc2c2ccccc12] IM-A5 | 50.5 |
| 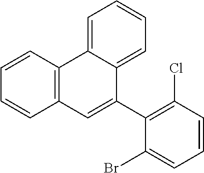 [SMILES: Clc1cccc(Br)c1-c1cc2ccccc2c2ccccc12] IM-a1 | 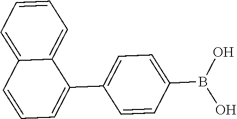 [SMILES: OB(O)c1ccc(-c2cccc3ccccc23)cc1] | 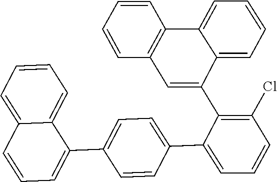 [SMILES: Clc1cccc(-c2ccc(-c3cccc4ccccc34)cc2)c1-c1cc2ccccc2c2ccccc12] IM-A6 | 68.4 |
| 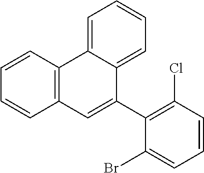 [SMILES: Clc1cccc(Br)c1-c1cc2ccccc2c2ccccc12] IM-a1 | 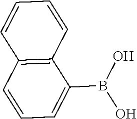 [SMILES: OB(O)c1cccc2ccccc12] | 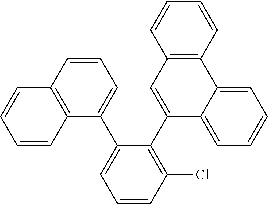 [SMILES: Clc1cccc(-c2cccc3ccccc23)c1-c1cc2ccccc2c2ccccc12] IM-A7 | 58.8 |
| 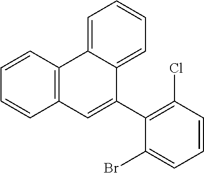 [SMILES: Clc1cccc(Br)c1-c1cc2ccccc2c2ccccc12] IM-a1 | 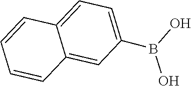 [SMILES: OB(O)c1ccc2ccccc2c1] | 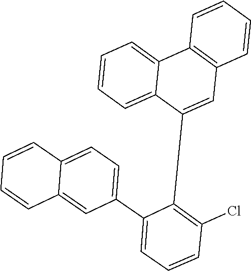 [SMILES: Clc1cccc(-c2ccc3ccccc3c2)c1-c1cc2ccccc2c2ccccc12] IM-A8 | 60.7 |
| 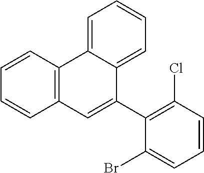 [SMILES: Clc1cccc(Br)c1-c1cc2ccccc2c2ccccc12] IM-a1 | 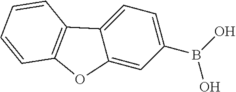 [SMILES: OB(O)c1ccc2c(c1)oc1ccccc12] | 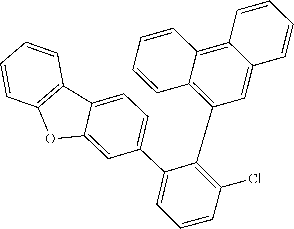 [SMILES: Clc1cccc(-c2ccc3c(c2)oc2ccccc23)c1-c1cc2ccccc2c2ccccc12] IM-A9 | 63.2 |
| 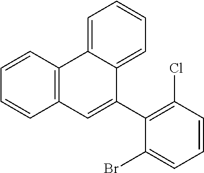 [SMILES: Clc1cccc(Br)c1-c1cc2ccccc2c2ccccc12] IM-a1 | 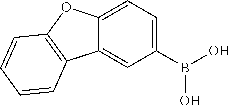 [SMILES: OB(O)c1ccc2oc3ccccc3c2c1] | 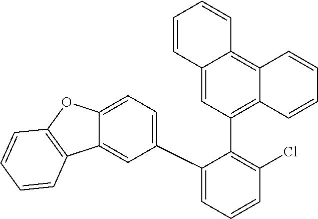 [SMILES: Clc1cccc(-c2ccc3oc4ccccc4c3c2)c1-c1cc2ccccc2c2ccccc12] IM-A10 | 62.5 |
| 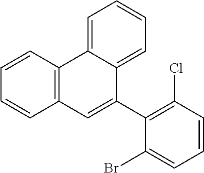 [SMILES: Clc1cccc(Br)c1-c1cc2ccccc2c2ccccc12] IM-a1 | 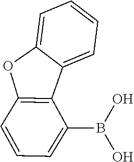 [SMILES: OB(O)c1cccc2oc3ccccc3c12] | 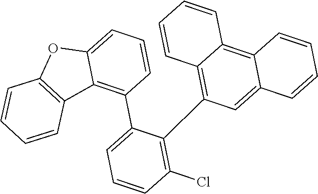 [SMILES: Clc1cccc(-c2cccc3oc4ccccc4c23)c1-c1cc2ccccc2c2ccccc12] IM-A11 | 48.4 |
| 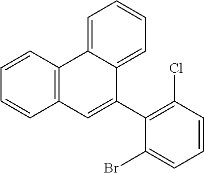 [SMILES: Clc1cccc(Br)c1-c1cc2ccccc2c2ccccc12] IM-a1 | 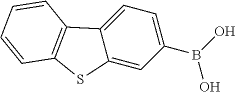 [SMILES: OB(O)c1ccc2c(c1)sc1ccccc12] | 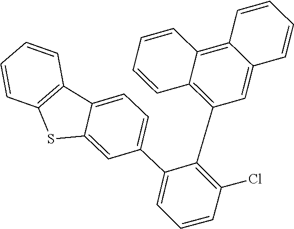 [SMILES: Clc1cccc(-c2ccc3c(c2)sc2ccccc23)c1-c1cc2ccccc2c2ccccc12] IM-A12 | 58.2 |
| 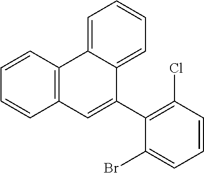 [SMILES: Clc1cccc(Br)c1-c1cc2ccccc2c2ccccc12] IM-a1 | 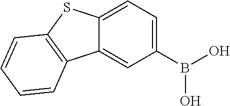 [SMILES: OB(O)c1ccc2sc3ccccc3c2c1] | 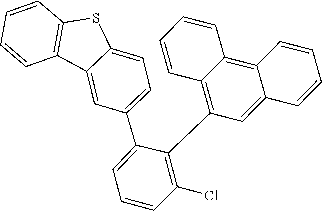 IM-A13 | 60.3 |
| 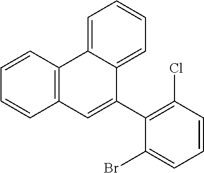 [SMILES: Clc1cccc(Br)c1-c1cc2ccccc2c2ccccc12] IM-a1 | 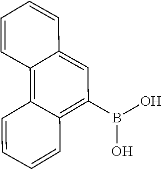 [SMILES: OB(O)c1cc2ccccc2c2ccccc12] | 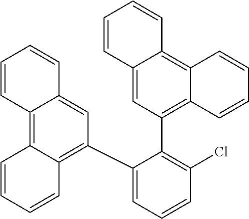 [SMILES: Clc1cccc(-c2cc3ccccc3c3ccccc23)c1-c1cc2ccccc2c2ccccc12] IM-A14 | 57.7 |
| 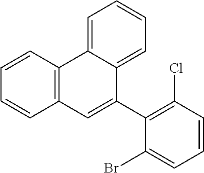 [SMILES: Clc1cccc(Br)c1-c1cc2ccccc2c2ccccc12] IM-a1 | 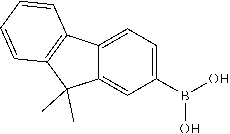 [SMILES: CC1(C)c2ccccc2-c2ccc(B(O)O)cc21] | 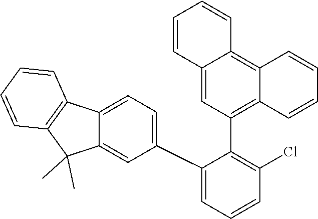 [SMILES: CC1(C)c2ccccc2-c2ccc(-c3cccc(Cl)c3-c3cc4ccccc4c4ccccc34)cc21] IM-A15 | 60.5 |
| 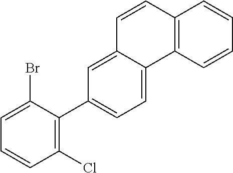 [SMILES: Clc1cccc(Br)c1-c1ccc2c(ccc3ccccc32)c1] IM-a2 | 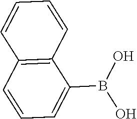 [SMILES: OB(O)c1cccc2ccccc12] | 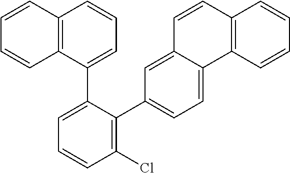 [SMILES: Clc1cccc(-c2cccc3ccccc23)c1-c1ccc2c(ccc3ccccc32)c1] IM-A16 | |
| 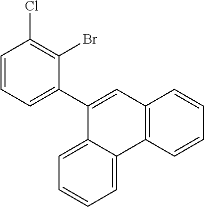 [SMILES: Clc1cccc(-c2cc3ccccc3c3ccccc23)c1Br] IM-b1 | 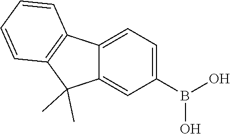 [SMILES: CC1(C)c2ccccc2-c2ccc(B(O)O)cc21] | 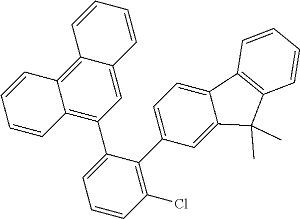 [SMILES: CC1(C)c2ccccc2-c2ccc(-c3c(Cl)cccc3-c3cc4ccccc4c4ccccc34)cc21] IM-B1 | 58.6 |
| 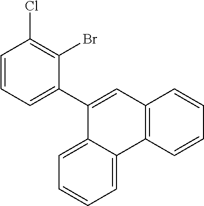 [SMILES: Clc1cccc(-c2cc3ccccc3c3ccccc23)c1Br] IM-b1 | 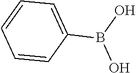 | 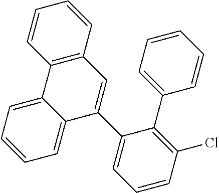 [SMILES: Clc1cccc(-c2cc3ccccc3c3ccccc23)c1-c1ccccc1] IM-B2 | 72.5 |
| 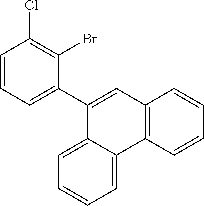 [SMILES: Clc1cccc(-c2cc3ccccc3c3ccccc23)c1Br] IM-b1 | 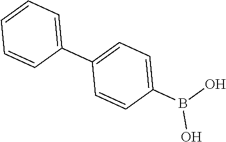 [SMILES: OB(O)c1ccc(-c2ccccc2)cc1] | 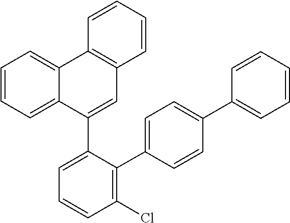 [SMILES: Clc1cccc(-c2cc3ccccc3c3ccccc23)c1-c1ccc(-c2ccccc2)cc1] IM-B3 | 69.9 |
| 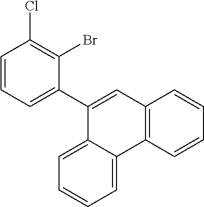 [SMILES: Clc1cccc(-c2cc3ccccc3c3ccccc23)c1Br] IM-b1 | 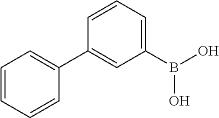 [SMILES: OB(O)c1cccc(-c2ccccc2)c1] | 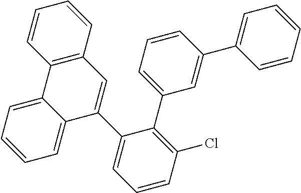 [SMILES: Clc1cccc(-c2cc3ccccc3c3ccccc23)c1-c1cccc(-c2ccccc2)c1] IM-B4 | 66.8 |
| 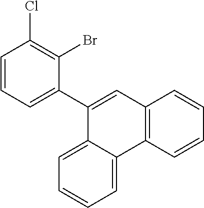 [SMILES: Clc1cccc(-c2cc3ccccc3c3ccccc23)c1Br] IM-b1 | 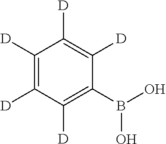 [SMILES: [2H]c1c([2H])c([2H])c(B(O)O)c([2H])c1[2H]] | 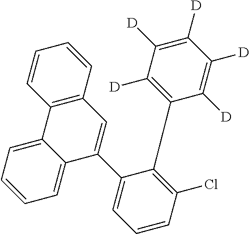 [SMILES: [2H]c1c([2H])c([2H])c(-c2c(Cl)cccc2-c2cc3ccccc3c3ccccc23)c([2H])c1[2H]] IM-B5 | 69.9 |
| 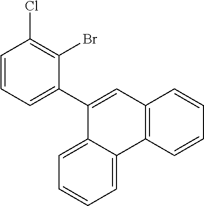 [SMILES: Clc1cccc(-c2cc3ccccc3c3ccccc23)c1Br] IM-b1 | 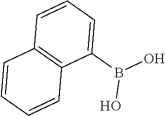 [SMILES: OB(O)c1cccc2ccccc12] | 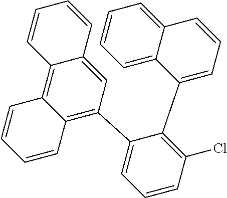 [SMILES: Clc1cccc(-c2cc3ccccc3c3ccccc23)c1-c1cccc2ccccc12] IM-B6 | 57.5 |
| 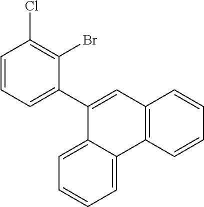 [SMILES: Clc1cccc(-c2cc3ccccc3c3ccccc23)c1Br] IM-b1 | 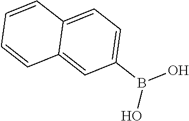 [SMILES: OB(O)c1ccc2ccccc2c1] | 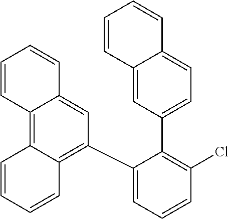 [SMILES: Clc1cccc(-c2cc3ccccc3c3ccccc23)c1-c1ccc2ccccc2c1] IM-B7 | 59.0 |
| 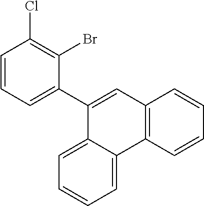 [SMILES: Clc1cccc(-c2cc3ccccc3c3ccccc23)c1Br] IM-b1 | 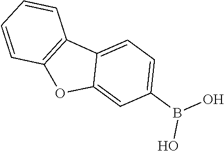 [SMILES: OB(O)c1ccc2c(c1)oc1ccccc12] | 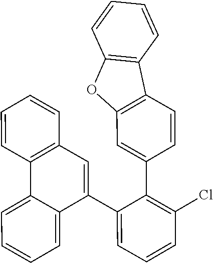 [SMILES: Clc1cccc(-c2cc3ccccc3c3ccccc23)c1-c1ccc2c(c1)oc1ccccc12] IM-B8 | 60.3 |
| 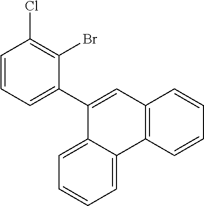 [SMILES: Clc1cccc(-c2cc3ccccc3c3ccccc23)c1Br] IM-b1 | 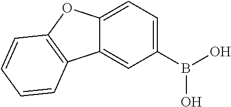 [SMILES: OB(O)c1ccc2oc3ccccc3c2c1] | 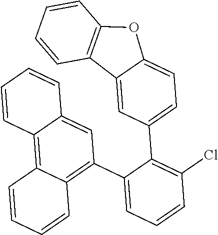 [SMILES: Clc1cccc(-c2cc3ccccc3c3ccccc23)c1-c1ccc2oc3ccccc3c2c1] IM-B9 | 58.1 |
| 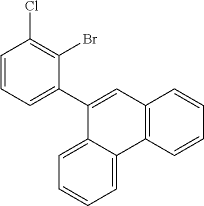 [SMILES: Clc1cccc(-c2cc3ccccc3c3ccccc23)c1Br] IM-b1 | 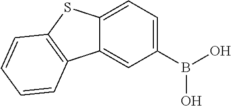 [SMILES: OB(O)c1ccc2sc3ccccc3c2c1] | 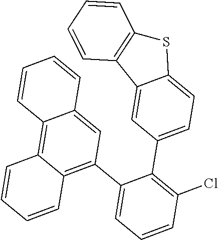 [SMILES: Clc1cccc(-c2cc3ccccc3c3ccccc23)c1-c1ccc2sc3ccccc3c2c1] IM-B10 | 57.4 |
| 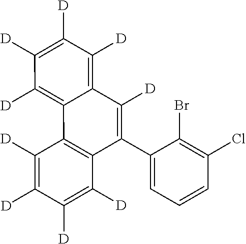 [SMILES: [2H]c1c([2H])c([2H])c2c(c1[2H])c([2H])c(-c1cccc(Cl)c1Br)c1c([2H])c([2H])c([2H])c([2H])c12] IM-b2 | 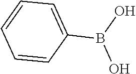 | 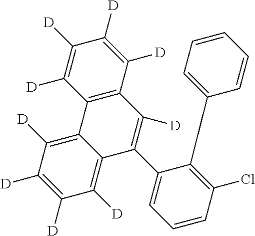 [SMILES: [2H]c1c([2H])c([2H])c2c(c1[2H])c([2H])c(-c1cccc(Cl)c1-c1ccccc1)c1c([2H])c([2H])c([2H])c([2H])c12] IM-B11 | 54.3 |
| 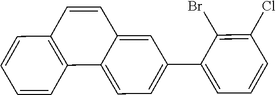 [SMILES: Clc1cccc(-c2ccc3c(ccc4ccccc43)c2)c1Br] IM-b3 | 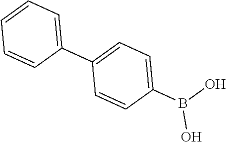 [SMILES: OB(O)c1ccc(-c2ccccc2)cc1] | 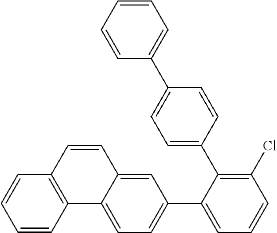 [SMILES: Clc1cccc(-c2ccc3c(ccc4ccccc43)c2)c1-c1ccc(-c2ccccc2)cc1] IM-B12 | 60.5 |
| 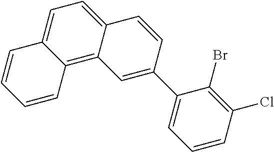 [SMILES: Clc1cccc(-c2ccc3ccc4ccccc4c3c2)c1Br] IM-b4 | 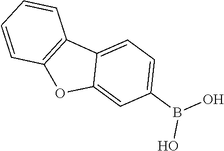 [SMILES: OB(O)c1ccc2c(c1)oc1ccccc12] | 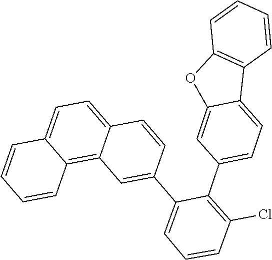 [SMILES: Clc1cccc(-c2ccc3ccc4ccccc4c3c2)c1-c1ccc2c(c1)oc1ccccc12] IM-B13 | 47.3 |
| 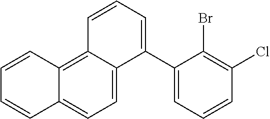 [SMILES: Clc1cccc(-c2cccc3c2ccc2ccccc23)c1Br] IM-b5 | 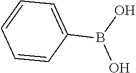 | 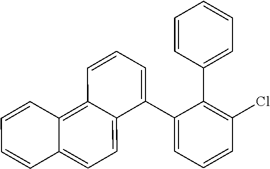 [SMILES: Clc1cccc(-c2cccc3c2ccc2ccccc23)c1-c1ccccc1] IM-B14 | 45.2 |

### 2. Synthesis of IM-C1 and IM-D1

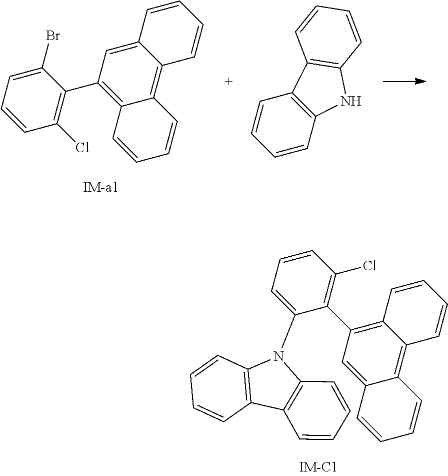 [SMILES: Clc1cccc(Br)c1-c1cc2ccccc2c2ccccc12 ; c1ccc2c(c1)[nH]c1ccccc12 ; Clc1cccc(-n2c3ccccc3c3ccccc32)c1-c1cc2ccccc2c2ccccc12]

Under a nitrogen atmosphere, 9H-carbazole (3.64 g, 21.76 mmol), IM-a1 (8.0 g, 21.67 mmol), cuprous iodide (0.83 g, 4.35 mmol), potassium carbonate (6.62 g, 47.88 mmol), 1,10-phenanthroline (1.57 g, 8.70 mmol), and 18-crown-6 (0.58 g, 2.18 mmol) were sequentially added to a 1 L three-necked flask, followed by the addition of 80 mL of DMF. The resulting mixture was maintained under a nitrogen atmosphere for 20 minutes, and then was slowly heated to reflux with stirring for 24 hours of reaction. The reaction solution was cooled to room temperature and added into 300 mL water to remove the DMF. Subsequently, the reaction solution was extracted with dichloromethane and then dried over anhydrous magnesium sulfate for 30 minutes. The solvent was then removed under reduced pressure, and the residue was chromatographed through a silica gel column using dichloromethane/petroleum ether (v/v=1:4) to yield IM-C1 as a grayish-white solid (6.80 g, yield 69.1%).

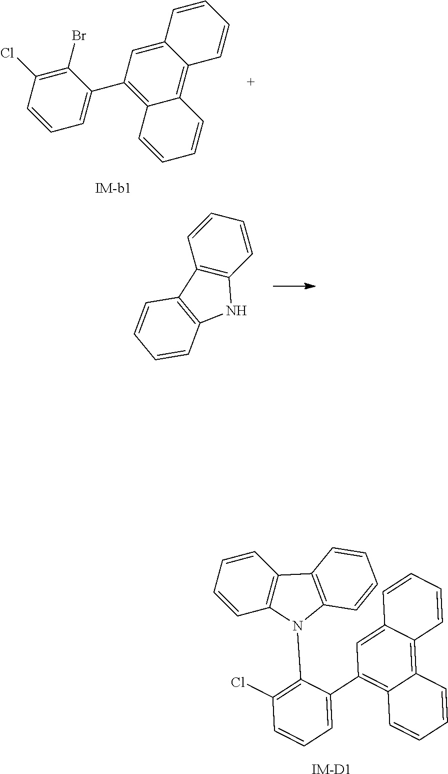

IM-D1 was synthesized using the same method as that for IM-C1, except that IM-bl was used instead of IM-a1, while the other raw materials and conditions remained unchanged, to yield IM-D1 (yield: 66.8%).

### 3. Synthesis of IM-N1:

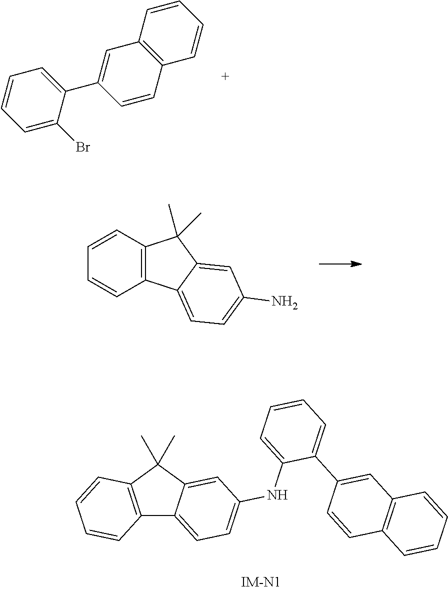 [SMILES: Brc1ccccc1-c1ccc2ccccc2c1 ; CC1(C)c2ccccc2-c2ccc(N)cc21 ; CC1(C)c2ccccc2-c2ccc(Nc3ccccc3-c3ccc4ccccc4c3)cc21]

Under a nitrogen atmosphere, 2-(2-bromophenyl)naphthalene (6.0 g, 21.19 mmol), 2-amino-9,9-dimethylfluorene (4.6 g, 21.97 mmol), tris(dibenzylidene acetone) dipalladium (0.2 g, 0.21 mmol), 2-dicyclohexylphosphino-2′,4′,6′-triisopropylbiphenyl (0.2 g, 0.42 mmol), sodium tert-butoxide (3.1 g, 32.31 mmol), and 60 mL toluene were added to a reaction flask. The resulting mixture was heated to 108° C. under a nitrogen atmosphere and refluxed with stirring for 3 hours; then the reaction solution was cooled to room temperature. The reaction solution was washed with water and dried over magnesium sulfate. After filtration, the filtrate was subjected to solvent removal under reduced pressure to yield a crude product as a yellow solid; the crude product was then purified by recrystallization from toluene/n-hexane to yield IM-N1 (6.5 g, yield 74.5%).

IM-Nx listed in Table 3 were synthesized using the same method as that for IM-N1, except that Raw material 5 was used instead of 2-(2-bromophenyl) naphthalene, and Raw material 6 was used instead of 2-amino-9,9-dimethylfluorene. The main raw materials utilized, the intermediates synthesized, and yields thereof are shown in Table 3.

TABLE 3

| Raw material 5 | Raw material 6 | IM-Nx | Yield/% |
|---|---|---|---|
| 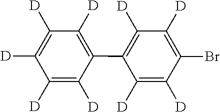 [SMILES: [2H]c1c([2H])c([2H])c(-c2c([2H])c([2H])c(Br)c([2H])c2[2H])c([2H])c1[2H]] | 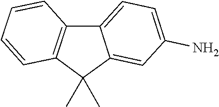 [SMILES: CC1(C)c2ccccc2-c2ccc(N)cc21] | 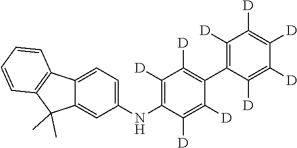 IM-N2 | 77.4 |
| 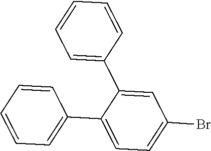 [SMILES: Brc1ccc(-c2ccccc2)c(-c2ccccc2)c1] | 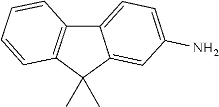 [SMILES: CC1(C)c2ccccc2-c2ccc(N)cc21] | 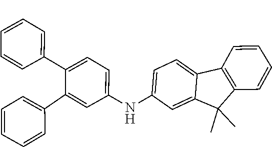 IM-N3 | 68.5 |
| 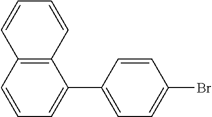 [SMILES: Brc1ccc(-c2cccc3ccccc23)cc1] | 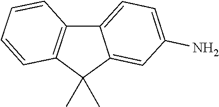 [SMILES: CC1(C)c2ccccc2-c2ccc(N)cc21] | 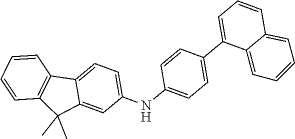 IM-N4 | 72.6 |
| 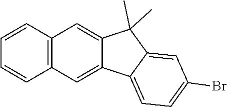 [SMILES: CC1(C)c2cc(Br)ccc2-c2cc3ccccc3cc21] | 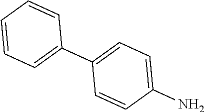 [SMILES: Nc1ccc(-c2ccccc2)cc1] | 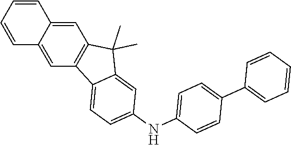 IM-N5 | 70.8 |
| 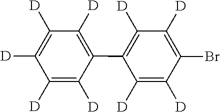 [SMILES: [2H]c1c([2H])c([2H])c(-c2c([2H])c([2H])c(Br)c([2H])c2[2H])c([2H])c1[2H]] | 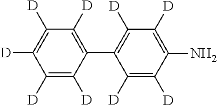 [SMILES: [2H]c1c([2H])c([2H])c(-c2c([2H])c([2H])c(N)c([2H])c2[2H])c([2H])c1[2H]] | 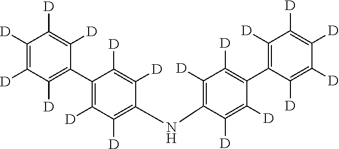 IM-N6 | 72.0 |
| 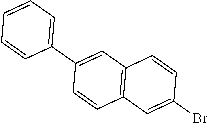 [SMILES: Brc1ccc2cc(-c3ccccc3)ccc2c1] | 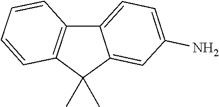 [SMILES: CC1(C)c2ccccc2-c2ccc(N)cc21] | 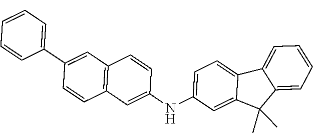 IM-N7 | 71.1 |
| 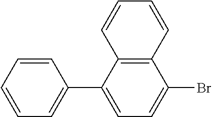 [SMILES: Brc1ccc(-c2ccccc2)c2ccccc12] | 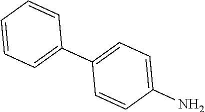 [SMILES: Nc1ccc(-c2ccccc2)cc1] | 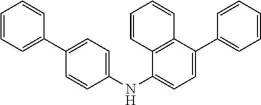 IM-N8 | 66.5 |
| 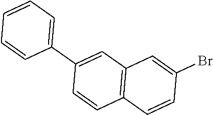 [SMILES: Brc1ccc2ccc(-c3ccccc3)cc2c1] | 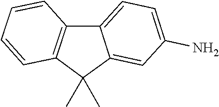 [SMILES: CC1(C)c2ccccc2-c2ccc(N)cc21] | 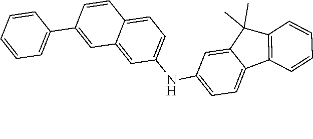 IM-N9 | 69.4 |

### 4. Synthesis of Compound 11

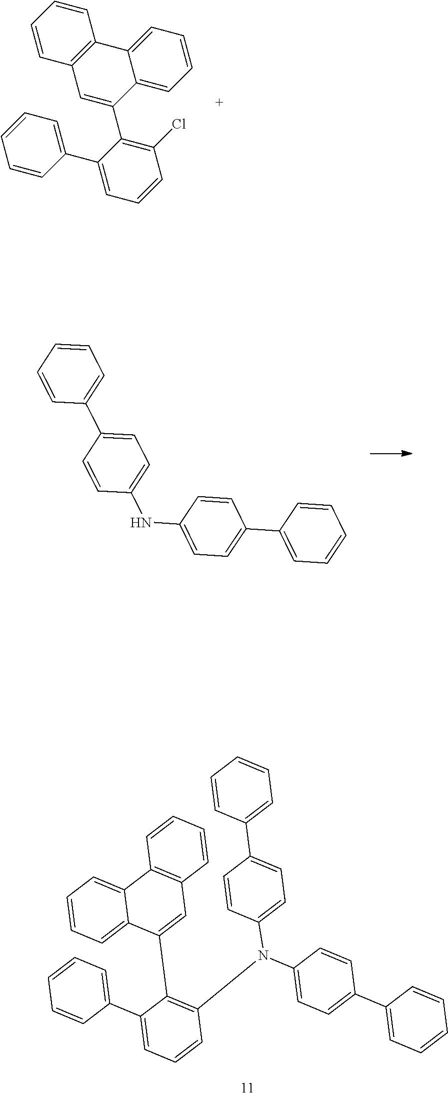 [SMILES: Clc1cccc(-c2ccccc2)c1-c1cc2ccccc2c2ccccc12 ; c1ccc(-c2ccc(Nc3ccc(-c4ccccc4)cc3)cc2)cc1 ; c1ccc(-c2ccc(N(c3ccc(-c4ccccc4)cc3)c3cccc(-c4ccccc4)c3-c3cc4ccccc4c4ccccc34)cc2)cc1]

Under a nitrogen atmosphere, IM-A1 (5.0 g, 13.70 mmol), bis(4-biphenyl)amine (4.4 g, 13.70 mmol), tris(dibenzylidene acetone) dipalladium (0.13 g, 0.14 mmol), 2-dicyclohexylphosphino-2′,6′-dimethoxybiphenyl (0.11 g, 0.27 mmol), and sodium tert-butoxide (1.97 g, 20.55 mmol) were sequentially added to a 100 mL three-necked flask, followed by adding into toluene (50 mL). The resulting mixture was heated to 108° C. under a nitrogen atmosphere and stirred for 4 hours; subsequently the reaction solution was cooled to room temperature. The reaction solution was washed with water and dried over magnesium sulfate. After filtration, the filtrate was subjected to solvent removal under reduced pressure to yield a crude product; the crude product was purified by recrystallization from toluene to yield Compound 11 as a white solid (4.8 g, yield 53.9%). Mass Spectra (m/z)=650.3[M+H]⁺.

The compounds listed in Table 4 were prepared using the same method as that for Compound 11, except that Raw material 7 was used instead of IM-A1, and Raw material 8 was used instead of aniline. The main raw materials utilized, the synthesized compounds, the yields, and mass spectra thereof are shown in Table 4.

TABLE 4

| | | | | | Mass |
|---|---|---|---|---|---|
| Synthesis | | | | | Spectra |
| Example | | | | | (m/z)/ |
| No. | Raw material 7 | Raw material 8 | Compound | Yield/% | [M + H]⁺ |
| 2 | 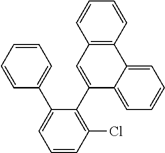 [SMILES: Clc1cccc(-c2ccccc2)c1-c1cc2ccccc2c2ccccc12] IM-A1 | 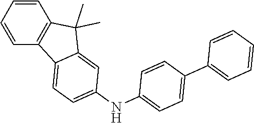 [SMILES: CC1(C)c2ccccc2-c2ccc(Nc3ccc(-c4ccccc4)cc3)cc21] CAS: 897671-69-1 | 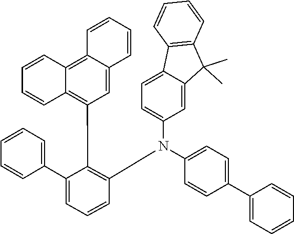 [SMILES: CC1(C)c2ccccc2-c2ccc(N(c3ccc(-c4ccccc4)cc3)c3cccc(-c4ccccc4)c3-c3cc4ccccc4c4ccccc34)cc21] 15 | 54.2 | 690.3 |
| 3 | 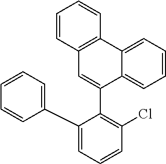 [SMILES: Clc1cccc(-c2ccccc2)c1-c1cc2ccccc2c2ccccc12] IM-A1 | 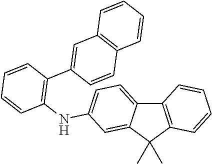 [SMILES: CC1(C)c2ccccc2-c2ccc(Nc3ccccc3-c3ccc4ccccc4c3)cc21] IM-N1 | 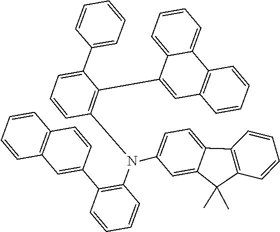 [SMILES: CC1(C)c2ccccc2-c2ccc(N(c3ccccc3-c3ccc4ccccc4c3)c3cccc(-c4ccccc4)c3-c3cc4ccccc4c4ccccc34)cc21] 20 | 39.4 | 740.3 |
| 4 | 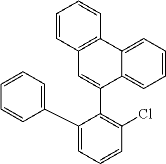 [SMILES: Clc1cccc(-c2ccccc2)c1-c1cc2ccccc2c2ccccc12] IM-A1 | 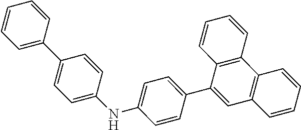 [SMILES: c1ccc(-c2ccc(Nc3ccc(-c4cc5ccccc5c5ccccc45)cc3)cc2)cc1] CAS: 1918982-76-9 | 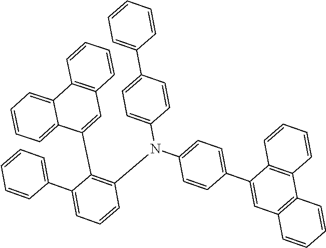 [SMILES: c1ccc(-c2ccc(N(c3ccc(-c4cc5ccccc5c5ccccc45)cc3)c3cccc(-c4ccccc4)c3-c3cc4ccccc4c4ccccc34)cc2)cc1] 43 | 50.2 | 750.3 |
| 5 | 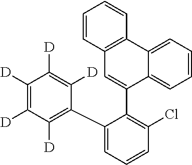 [SMILES: [2H]c1c([2H])c([2H])c(-c2cccc(Cl)c2-c2cc3ccccc3c3ccccc23)c([2H])c1[2H]] IM-A2 | 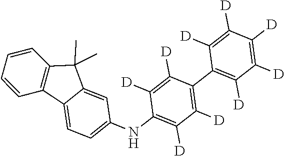 [SMILES: [2H]c1c([2H])c([2H])c(-c2c([2H])c([2H])c(Nc3ccc4c(c3)C(C)(C)c3ccccc3-4)c([2H])c2[2H])c([2H])c1[2H]] IM-N2 | 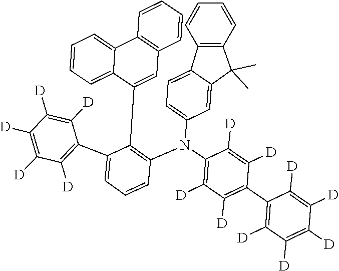 [SMILES: [2H]c1c([2H])c([2H])c(-c2cccc(N(c3ccc4c(c3)C(C)(C)c3ccccc3-4)c3c([2H])c([2H])c(-c4c([2H])c([2H])c([2H])c([2H])c4[2H])c([2H])c3[2H])c2-c2cc3ccccc3c3ccccc23)c([2H])c1[2H]] 53 | 49.0 | 704.4 |
| 6 | 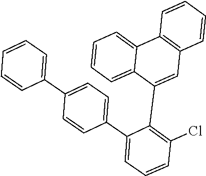 [SMILES: Clc1cccc(-c2ccc(-c3ccccc3)cc2)c1-c1cc2ccccc2c2ccccc12] IM-A3 | 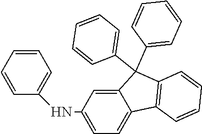 [SMILES: c1ccc(Nc2ccc3c(c2)C(c2ccccc2)(c2ccccc2)c2ccccc2-3)cc1] CAS: 860465-14-1 | 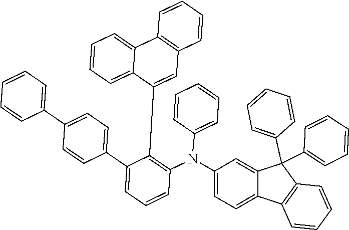 [SMILES: c1ccc(-c2ccc(-c3cccc(N(c4ccccc4)c4ccc5c(c4)C(c4ccccc4)(c4ccccc4)c4ccccc4-5)c3-c3cc4ccccc4c4ccccc34)cc2)cc1] 65 | 46.1 | 814.3 |
| 7 | 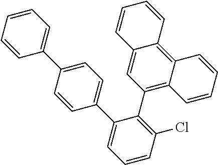 [SMILES: Clc1cccc(-c2ccc(-c3ccccc3)cc2)c1-c1cc2ccccc2c2ccccc12] IM-A3 | 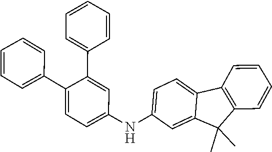 IM-N3 | 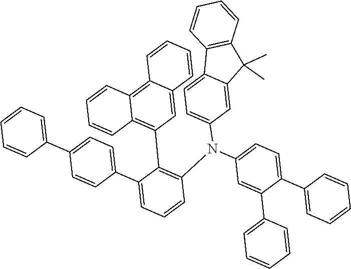 [SMILES: CC1(C)c2ccccc2-c2ccc(N(c3ccc(-c4ccccc4)c(-c4ccccc4)c3)c3cccc(-c4ccc(-c5ccccc5)cc4)c3-c3cc4ccccc4c4ccccc34)cc21] 79 | 42.2 | 842.4 |
| 8 | 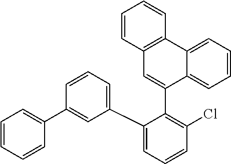 [SMILES: Clc1cccc(-c2cccc(-c3ccccc3)c2)c1-c1cc2ccccc2c2ccccc12] IM-A4 | 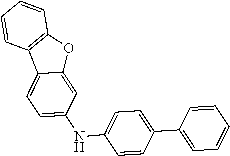 [SMILES: c1ccc(-c2ccc(Nc3ccc4c(c3)oc3ccccc34)cc2)cc1] CAS: 1290039-85-8 | 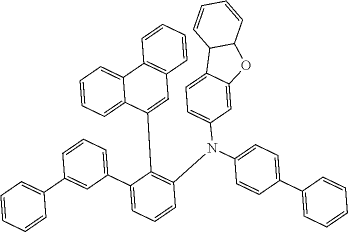 [SMILES: C1=CC2Oc3cc(N(c4ccc(-c5ccccc5)cc4)c4cccc(-c5cccc(-c6ccccc6)c5)c4-c4cc5ccccc5c5ccccc45)ccc3C2C=C1] 100 | 49.3 | 740.3 |
| 9 | 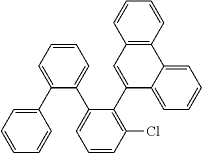 [SMILES: Clc1cccc(-c2ccccc2-c2ccccc2)c1-c1cc2ccccc2c2ccccc12] IM-A5 | 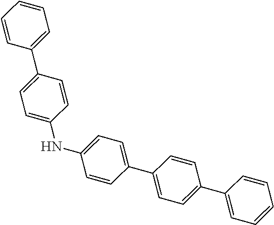 [SMILES: c1ccc(-c2ccc(Nc3ccc(-c4ccc(-c5ccccc5)cc4)cc3)cc2)cc1] CAS: 897921-63-0 | 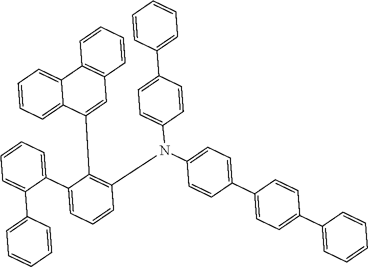 [SMILES: c1ccc(-c2ccc(-c3ccc(N(c4ccc(-c5ccccc5)cc4)c4cccc(-c5ccccc5-c5ccccc5)c4-c4cc5ccccc5c5ccccc45)cc3)cc2)cc1] 130 | 53.5 | 802.3 |
| 10 | 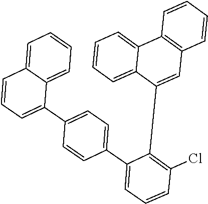 [SMILES: Clc1cccc(-c2ccc(-c3cccc4ccccc34)cc2)c1-c1cc2ccccc2c2ccccc12] IM-A6 | 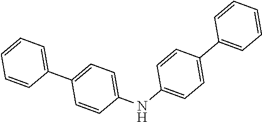 [SMILES: c1ccc(-c2ccc(Nc3ccc(-c4ccccc4)cc3)cc2)cc1] CAS: 102113-98-4 | 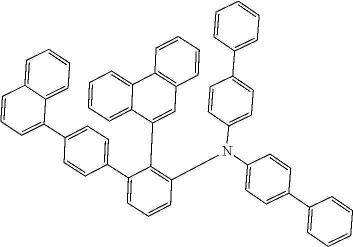 [SMILES: c1ccc(-c2ccc(N(c3ccc(-c4ccccc4)cc3)c3cccc(-c4ccc(-c5cccc6ccccc56)cc4)c3-c3cc4ccccc4c4ccccc34)cc2)cc1] 141 | 51.4 | 776.3 |
| 11 | 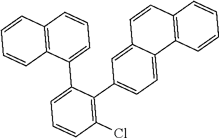 [SMILES: Clc1cccc(-c2cccc3ccccc23)c1-c1ccc2c(ccc3ccccc32)c1] IM-A16 | 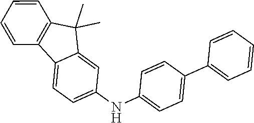 [SMILES: CC1(C)c2ccccc2-c2ccc(Nc3ccc(-c4ccccc4)cc3)cc21] CAS: 897671-69-1 | 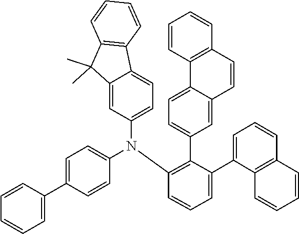 [SMILES: CC1(C)c2ccccc2-c2ccc(N(c3ccc(-c4ccccc4)cc3)c3cccc(-c4cccc5ccccc45)c3-c3ccc4c(ccc5ccccc54)c3)cc21] 160 | 50.3 | 740.3 |
| 12 | 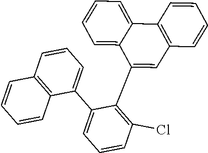 [SMILES: Clc1cccc(-c2cccc3ccccc23)c1-c1cc2ccccc2c2ccccc12] IM-A7 | 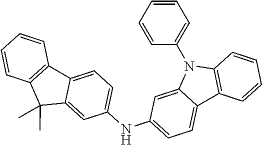 CAS: 1456702-57-0 | 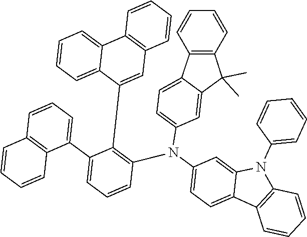 [SMILES: CC1(C)c2ccccc2-c2ccc(N(c3ccc4c5ccccc5n(-c5ccccc5)c4c3)c3cccc(-c4cccc5ccccc45)c3-c3cc4ccccc4c4ccccc34)cc21] 173 | 47.4 | 829.4 |
| 13 | 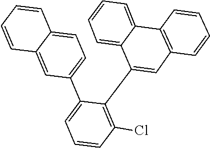 [SMILES: Clc1cccc(-c2ccc3ccccc3c2)c1-c1cc2ccccc2c2ccccc12] IM-A8 | 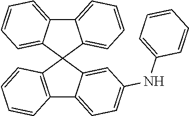 CAS: 1257247-93-0 | 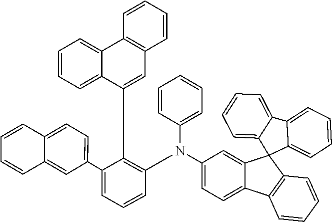 [SMILES: c1ccc(N(c2ccc3c(c2)C2(c4ccccc4-c4ccccc42)c2ccccc2-3)c2cccc(-c3ccc4ccccc4c3)c2-c2cc3ccccc3c3ccccc23)cc1] 190 | 46.6 | 786.3 |
| 14 | 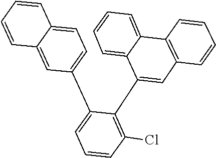 [SMILES: Clc1cccc(-c2ccc3ccccc3c2)c1-c1cc2ccccc2c2ccccc12] IM-A8 | 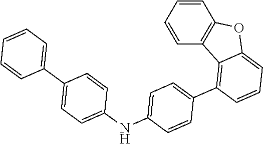 [SMILES: c1ccc(-c2ccc(Nc3ccc(-c4cccc5oc6ccccc6c45)cc3)cc2)cc1] CAS: 1922919-50-3 | 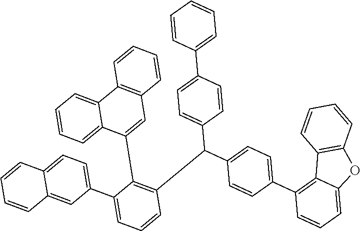 [SMILES: c1ccc(-c2ccc(C(c3ccc(-c4cccc5oc6ccccc6c45)cc3)c3cccc(-c4ccc5ccccc5c4)c3-c3cc4ccccc4c4ccccc34)cc2)cc1] 201 | 49.2 | 790.3 |
| 15 | 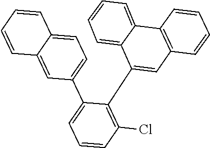 [SMILES: Clc1cccc(-c2ccc3ccccc3c2)c1-c1cc2ccccc2c2ccccc12] IM-A8 | 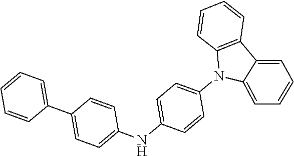 [SMILES: c1ccc(-c2ccc(Nc3ccc(-n4c5ccccc5c5ccccc54)cc3)cc2)cc1] CAS: 1210470-43-1 | 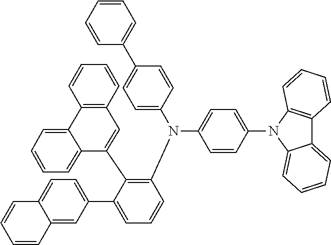 [SMILES: c1ccc(-c2ccc(N(c3ccc(-n4c5ccccc5c5ccccc54)cc3)c3cccc(-c4ccc5ccccc5c4)c3-c3cc4ccccc4c4ccccc34)cc2)cc1] 213 | 51.7 | 789.3 |
| 16 | 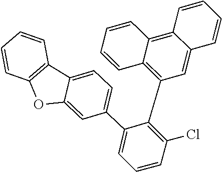 [SMILES: Clc1cccc(-c2ccc3c(c2)oc2ccccc23)c1-c1cc2ccccc2c2ccccc12] IM-A9 | 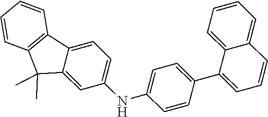 IM-N4 | 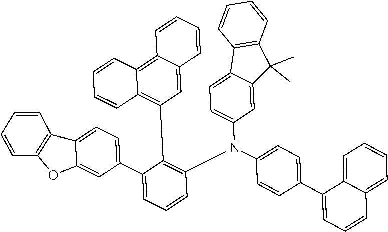 [SMILES: CC1(C)c2ccccc2-c2ccc(N(c3ccc(-c4cccc5ccccc45)cc3)c3cccc(-c4ccc5c(c4)oc4ccccc45)c3-c3cc4ccccc4c4ccccc34)cc21] 222 | 48.3 | 830.3 |
| 17 | 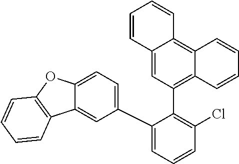 [SMILES: Clc1cccc(-c2ccc3oc4ccccc4c3c2)c1-c1cc2ccccc2c2ccccc12] IM-A10 | 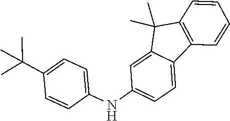 [SMILES: CC(C)(C)c1ccc(Nc2ccc3c(c2)C(C)(C)c2ccccc2-3)cc1] CAS: 944418-46-6 | 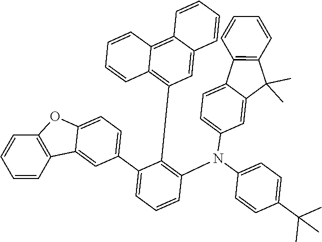 236 | 52.6 | 760.4 |
| 18 | 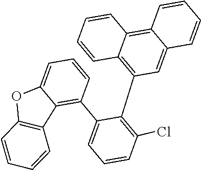 [SMILES: Clc1cccc(-c2cccc3oc4ccccc4c23)c1-c1cc2ccccc2c2ccccc12] IM-A11 | 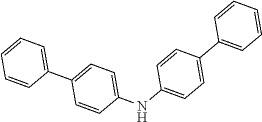 [SMILES: c1ccc(-c2ccc(Nc3ccc(-c4ccccc4)cc3)cc2)cc1] CAS: 102113-98-4 | 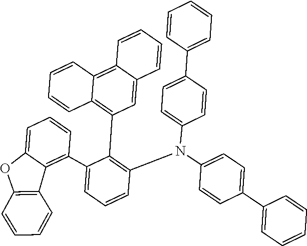 [SMILES: c1ccc(-c2ccc(N(c3ccc(-c4ccccc4)cc3)c3cccc(-c4cccc5oc6ccccc6c45)c3-c3cc4ccccc4c4ccccc34)cc2)cc1] 250 | 54.0 | 740.3 |
| 19 | 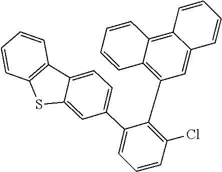 [SMILES: Clc1cccc(-c2ccc3c(c2)sc2ccccc23)c1-c1cc2ccccc2c2ccccc12] IM-A12 | 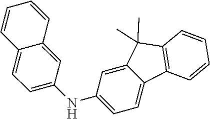 [SMILES: CC1(C)c2ccccc2-c2ccc(Nc3ccc4ccccc4c3)cc21] CAS: 1263001-82-6 | 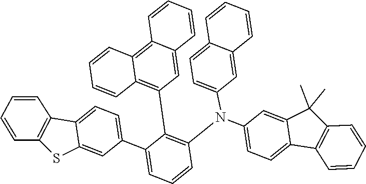 [SMILES: CC1(C)c2ccccc2-c2ccc(N(c3ccc4ccccc4c3)c3cccc(-c4ccc5c(c4)sc4ccccc45)c3-c3cc4ccccc4c4ccccc34)cc21] 253 | 45.8 | 770.3 |
| 20 | 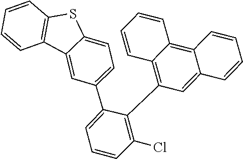 IM-A13 | 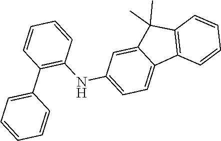 [SMILES: CC1(C)c2ccccc2-c2ccc(Nc3ccccc3-c3ccccc3)cc21] CAS: 1198395-24-2 | 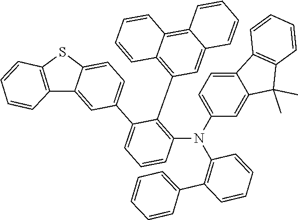 [SMILES: CC1(C)c2ccccc2-c2ccc(N(c3ccccc3-c3ccccc3)c3cccc(-c4ccc5sc6ccccc6c5c4)c3-c3cc4ccccc4c4ccccc34)cc21] 267 | 39.5 | 796.3 |
| 21 | 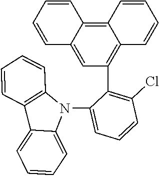 IM-C1 | 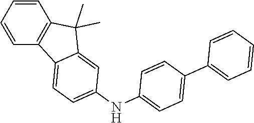 [SMILES: CC1(C)c2ccccc2-c2ccc(Nc3ccc(-c4ccccc4)cc3)cc21] CAS: 897671-69-1 | 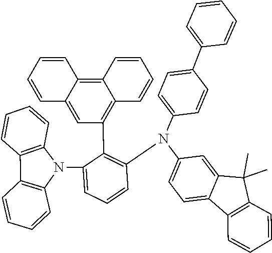 310 | 46.0 | 779.3 |
| 22 | 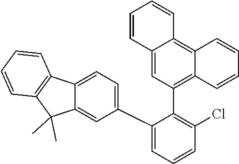 [SMILES: CC1(C)c2ccccc2-c2ccc(-c3cccc(Cl)c3-c3cc4ccccc4c4ccccc34)cc21] IM-A15 | 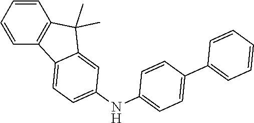 [SMILES: CC1(C)c2ccccc2-c2ccc(Nc3ccc(-c4ccccc4)cc3)cc21] CAS: 897671-69-1 | 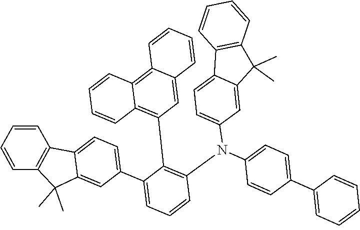 330 | 46.7 | 860.4 |
| 23 | 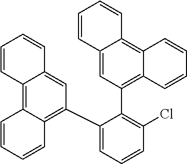 [SMILES: Clc1cccc(-c2cc3ccccc3c3ccccc23)c1-c1cc2ccccc2c2ccccc12] IM-A14 | 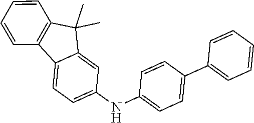 [SMILES: CC1(C)c2ccccc2-c2ccc(Nc3ccc(-c4ccccc4)cc3)cc21] CAS: 897671-69-1 | 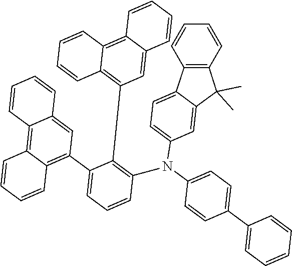 [SMILES: CC1(C)c2ccccc2-c2ccc(N(c3ccc(-c4ccccc4)cc3)c3cccc(-c4cc5ccccc5c5ccccc45)c3-c3cc4ccccc4c4ccccc34)cc21] 288 | 47.8 | 790.3 |
| 24 | 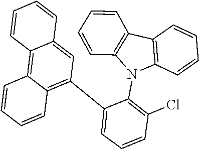 IM-D1 | 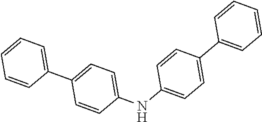 [SMILES: c1ccc(-c2ccc(Nc3ccc(-c4ccccc4)cc3)cc2)cc1] CAS: 102113-98-4 | 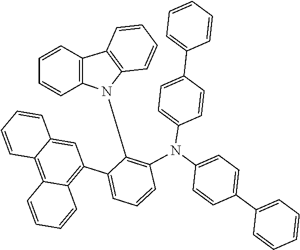 321 | 50.5 | 739.3 |
| 25 | 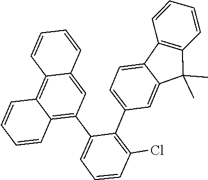 [SMILES: CC1(C)c2ccccc2-c2ccc(-c3c(Cl)cccc3-c3cc4ccccc4c4ccccc34)cc21] IM-B1 | 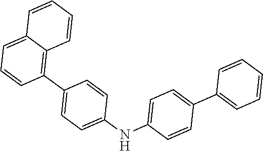 [SMILES: c1ccc(-c2ccc(Nc3ccc(-c4cccc5ccccc45)cc3)cc2)cc1] CAS: 897921-59-4 | 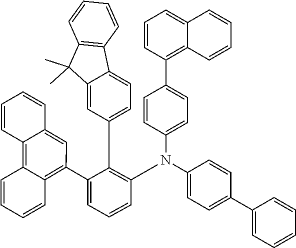 [SMILES: CC1(C)c2ccccc2-c2ccc(-c3c(-c4cc5ccccc5c5ccccc45)cccc3N(c3ccc(-c4ccccc4)cc3)c3ccc(-c4cccc5ccccc45)cc3)cc21] 345 | 47.4 | 816.4 |
| 26 | 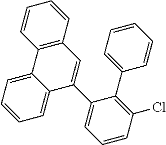 [SMILES: Clc1cccc(-c2cc3ccccc3c3ccccc23)c1-c1ccccc1] IM-B2 | 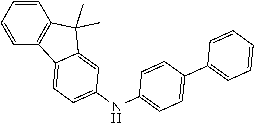 [SMILES: CC1(C)c2ccccc2-c2ccc(Nc3ccc(-c4ccccc4)cc3)cc21] CAS: 897671-69-1 | 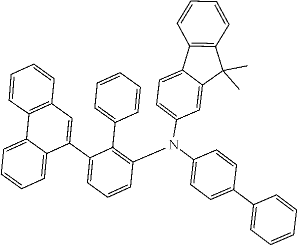 [SMILES: CC1(C)c2ccccc2-c2ccc(N(c3ccc(-c4ccccc4)cc3)c3cccc(-c4cc5ccccc5c5ccccc45)c3-c3ccccc3)cc21] 358 | 49.4 | 690.3 |
| 27 | 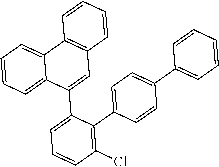 [SMILES: Clc1cccc(-c2cc3ccccc3c3ccccc23)c1-c1ccc(-c2ccccc2)cc1] IM-B3 | 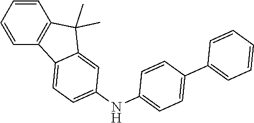 [SMILES: CC1(C)c2ccccc2-c2ccc(Nc3ccc(-c4ccccc4)cc3)cc21] CAS: 897671-69-1 | 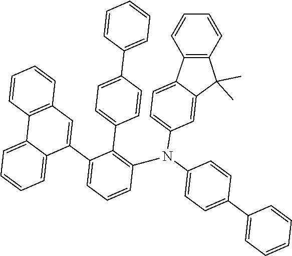 [SMILES: CC1(C)c2ccccc2-c2ccc(N(c3ccc(-c4ccccc4)cc3)c3cccc(-c4cc5ccccc5c5ccccc45)c3-c3ccc(-c4ccccc4)cc3)cc21] 395 | 47.9 | 766.3 |
| 28 | 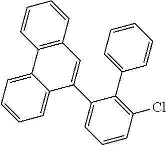 [SMILES: Clc1cccc(-c2cc3ccccc3c3ccccc23)c1-c1ccccc1] IM-B2 | 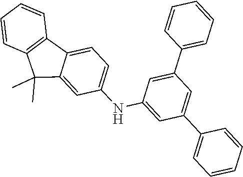 [SMILES: CC1(C)c2ccccc2-c2ccc(Nc3cc(-c4ccccc4)cc(-c4ccccc4)c3)cc21] CAS: 1372778-68-1 | 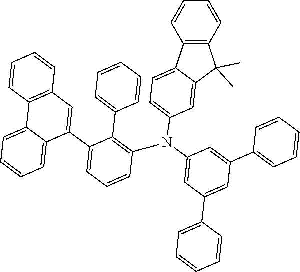 [SMILES: CC1(C)c2ccccc2-c2ccc(N(c3cc(-c4ccccc4)cc(-c4ccccc4)c3)c3cccc(-c4cc5ccccc5c5ccccc45)c3-c3ccccc3)cc21] 360 | 46.9 | 766.3 |
| 29 | 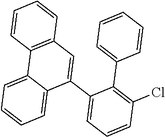 [SMILES: Clc1cccc(-c2cc3ccccc3c3ccccc23)c1-c1ccccc1] IM-B2 | 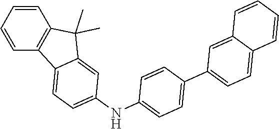 [SMILES: CC1(C)c2ccccc2-c2ccc(Nc3ccc(-c4ccc5ccccc5c4)cc3)cc21] CAS: 1456702-54-7 | 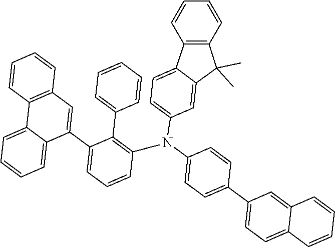 [SMILES: CC1(C)c2ccccc2-c2ccc(N(c3ccc(-c4ccc5ccccc5c4)cc3)c3cccc(-c4cc5ccccc5c5ccccc45)c3-c3ccccc3)cc21] 363 | 43.5 | 740.3 |
| 30 | 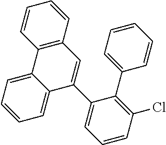 [SMILES: Clc1cccc(-c2cc3ccccc3c3ccccc23)c1-c1ccccc1] IM-B2 | 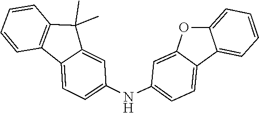 [SMILES: CC1(C)c2ccccc2-c2ccc(Nc3ccc4c(c3)oc3ccccc34)cc21] CAS: 1427556-50-0 | 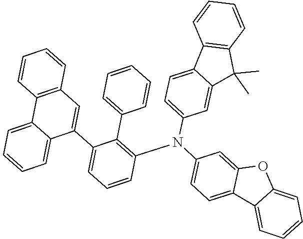 380 | 45.2 | 704.3 |
| 31 | 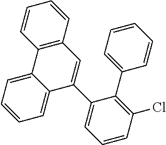 [SMILES: Clc1cccc(-c2cc3ccccc3c3ccccc23)c1-c1ccccc1] IM-B2 | 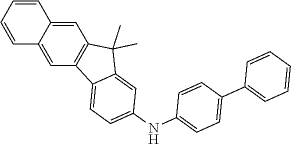 IM-N5 | 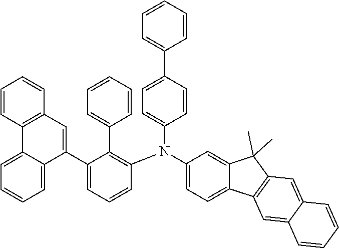 [SMILES: CC1(C)c2cc(N(c3ccc(-c4ccccc4)cc3)c3cccc(-c4cc5ccccc5c5ccccc45)c3-c3ccccc3)ccc2-c2cc3ccccc3cc21] 381 | 43.6 | 740.3 |
| 32 | 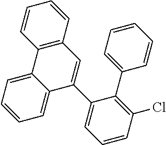 [SMILES: Clc1cccc(-c2cc3ccccc3c3ccccc23)c1-c1ccccc1] IM-B2 | 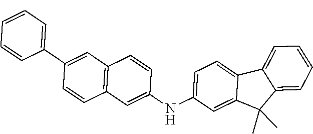 IM-N7 | 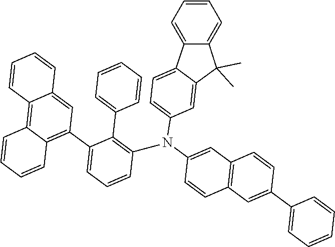 [SMILES: CC1(C)c2ccccc2-c2ccc(N(c3ccc4cc(-c5ccccc5)ccc4c3)c3cccc(-c4cc5ccccc5c5ccccc45)c3-c3ccccc3)cc21] 576 | 41.3 | 740.3 |
| 33 | 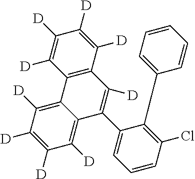 [SMILES: [2H]c1c([2H])c([2H])c2c(c1[2H])c([2H])c(-c1cccc(Cl)c1-c1ccccc1)c1c([2H])c([2H])c([2H])c([2H])c12] IM-B11 | 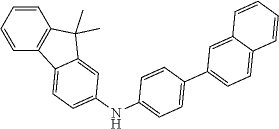 [SMILES: CC1(C)c2ccccc2-c2ccc(Nc3ccc(-c4ccc5ccccc5c4)cc3)cc21] CAS: 1456702-54-7 | 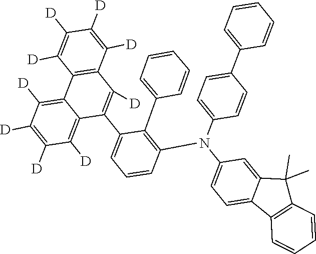 [SMILES: [2H]c1c([2H])c([2H])c2c(c1[2H])c([2H])c(-c1cccc(N(c3ccc(-c4ccccc4)cc3)c3ccc4c(c3)C(C)(C)c3ccccc3-4)c1-c1ccccc1)c1c([2H])c([2H])c([2H])c([2H])c12] 386 | 44.3 | 699.4 |
| 34 | 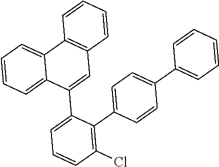 [SMILES: Clc1cccc(-c2cc3ccccc3c3ccccc23)c1-c1ccc(-c2ccccc2)cc1] IM-B3 | 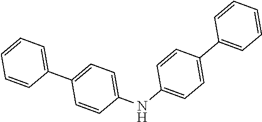 [SMILES: c1ccc(-c2ccc(Nc3ccc(-c4ccccc4)cc3)cc2)cc1] CAS: 102113-98-4 | 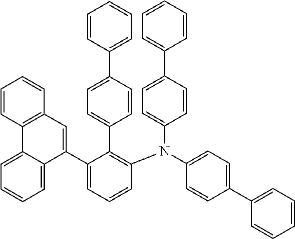 [SMILES: c1ccc(-c2ccc(-c3c(-c4cc5ccccc5c5ccccc45)cccc3N(c3ccc(-c4ccccc4)cc3)c3ccc(-c4ccccc4)cc3)cc2)cc1] 391 | 51.4 | 726.3 |
| 35 | 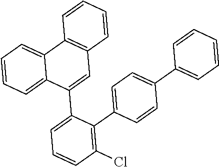 [SMILES: Clc1cccc(-c2cc3ccccc3c3ccccc23)c1-c1ccc(-c2ccccc2)cc1] IM-B3 | 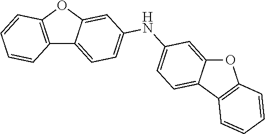 [SMILES: c1ccc2c(c1)oc1cc(Nc3ccc4c(c3)oc3ccccc34)ccc12] CAS: 1705595-86-3 | 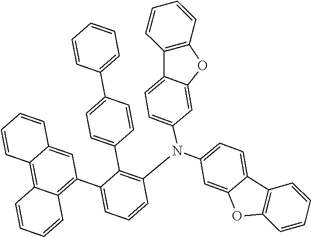 [SMILES: c1ccc(-c2ccc(-c3c(-c4cc5ccccc5c5ccccc45)cccc3N(c3ccc4c(c3)oc3ccccc34)c3ccc4c(c3)oc3ccccc34)cc2)cc1] 417 | 47.3 | 754.3 |
| 36 | 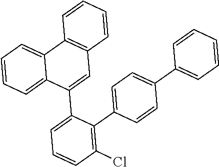 [SMILES: Clc1cccc(-c2cc3ccccc3c3ccccc23)c1-c1ccc(-c2ccccc2)cc1] IM-B3 | 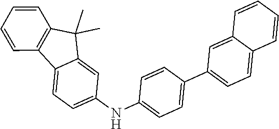 [SMILES: CC1(C)c2ccccc2-c2ccc(Nc3ccc(-c4ccc5ccccc5c4)cc3)cc21] CAS: 1456702-54-7 | 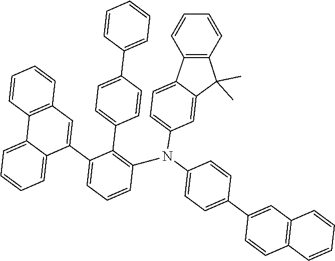 [SMILES: CC1(C)c2ccccc2-c2ccc(N(c3ccc(-c4ccc5ccccc5c4)cc3)c3cccc(-c4cc5ccccc5c5ccccc45)c3-c3ccc(-c4ccccc4)cc3)cc21] 398 | 43.8 | 816.4 |
| 37 | 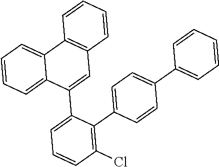 [SMILES: Clc1cccc(-c2cc3ccccc3c3ccccc23)c1-c1ccc(-c2ccccc2)cc1] IM-B3 | 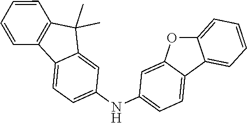 [SMILES: CC1(C)c2ccccc2-c2ccc(Nc3ccc4c(c3)oc3ccccc34)cc21] CAS: 1427556-50-0 | 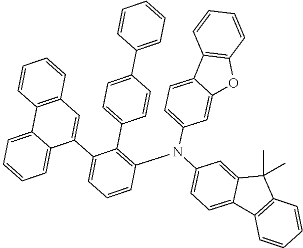 408 | 43.3 | 780.3 |
| 38 | 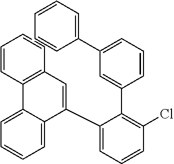 [SMILES: Clc1cccc(-c2cc3ccccc3c3ccccc23)c1-c1cccc(-c2ccccc2)c1] IM-B4 | 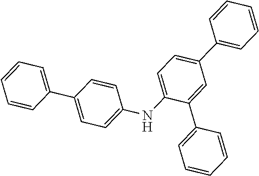 [SMILES: c1ccc(-c2ccc(Nc3ccc(-c4ccccc4)cc3-c3ccccc3)cc2)cc1] CAS: 1547491-85-9 | 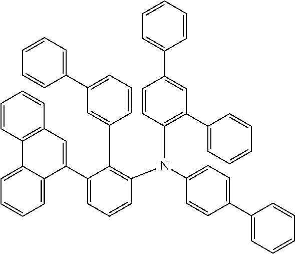 [SMILES: c1ccc(-c2ccc(N(c3ccc(-c4ccccc4)cc3-c3ccccc3)c3cccc(-c4cc5ccccc5c5ccccc45)c3-c3cccc(-c4ccccc4)c3)cc2)cc1] 432 | 43.6 | 802.3 |
| 39 | 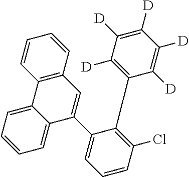 [SMILES: [2H]c1c([2H])c([2H])c(-c2c(Cl)cccc2-c2cc3ccccc3c3ccccc23)c([2H])c1[2H]] IM-B5 | 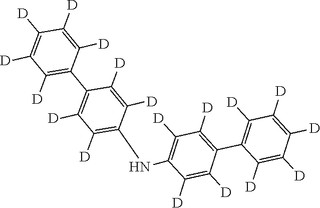 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] IM-N6 | 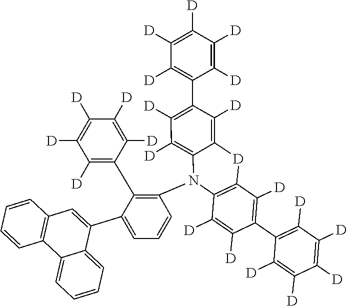 450 | 45.3 | 673.4 |
| 40 | 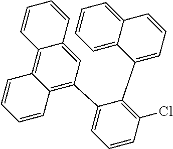 [SMILES: Clc1cccc(-c2cc3ccccc3c3ccccc23)c1-c1cccc2ccccc12] IM-B6 | 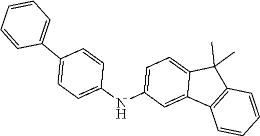 [SMILES: CC1(C)c2ccccc2-c2cc(Nc3ccc(-c4ccccc4)cc3)ccc21] CAS: 1326137-97-6 | 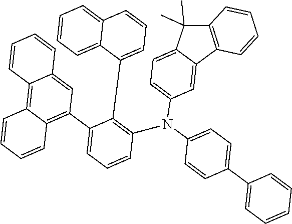 [SMILES: CC1(C)c2ccccc2-c2cc(N(c3ccc(-c4ccccc4)cc3)c3cccc(-c4cc5ccccc5c5ccccc45)c3-c3cccc4ccccc34)ccc21] 460 | 46.5 | 740.3 |
| 41 | 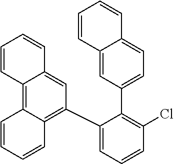 [SMILES: Clc1cccc(-c2cc3ccccc3c3ccccc23)c1-c1ccc2ccccc2c1] IM-B7 | 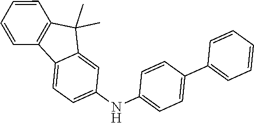 [SMILES: CC1(C)c2ccccc2-c2ccc(Nc3ccc(-c4ccccc4)cc3)cc21] CAS: 897671-69-1 | 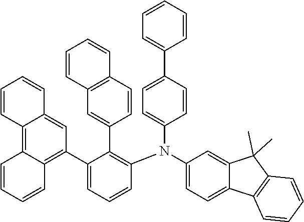 [SMILES: CC1(C)c2ccccc2-c2ccc(N(c3ccc(-c4ccccc4)cc3)c3cccc(-c4cc5ccccc5c5ccccc45)c3-c3ccc4ccccc4c3)cc21] 485 | 48.3 | 740.3 |
| 42 | 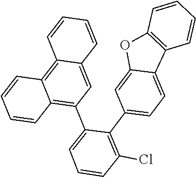 [SMILES: Clc1cccc(-c2cc3ccccc3c3ccccc23)c1-c1ccc2c(c1)oc1ccccc12] IM-B8 | 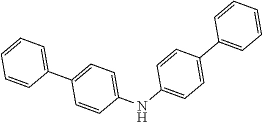 [SMILES: c1ccc(-c2ccc(Nc3ccc(-c4ccccc4)cc3)cc2)cc1] CAS: 102113-98-4 | 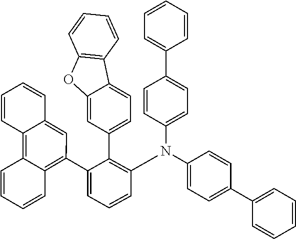 [SMILES: c1ccc(-c2ccc(N(c3ccc(-c4ccccc4)cc3)c3cccc(-c4cc5ccccc5c5ccccc45)c3-c3ccc4c(c3)oc3ccccc34)cc2)cc1] 506 | 51.2 | 740.3 |
| 43 | 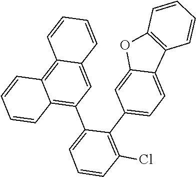 [SMILES: Clc1cccc(-c2cc3ccccc3c3ccccc23)c1-c1ccc2c(c1)oc1ccccc12] IM-B8 | 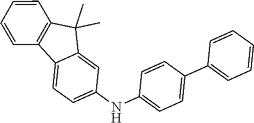 [SMILES: CC1(C)c2ccccc2-c2ccc(Nc3ccc(-c4ccccc4)cc3)cc21] CAS: 897671-69-1 | 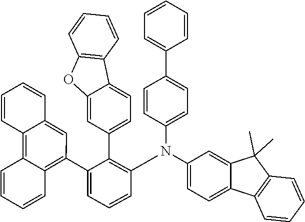 [SMILES: CC1(C)c2ccccc2-c2ccc(N(c3ccc(-c4ccccc4)cc3)c3cccc(-c4cc5ccccc5c5ccccc45)c3-c3ccc4c(c3)oc3ccccc34)cc21] 510 | 46.4 | 780.3 |
| 44 | 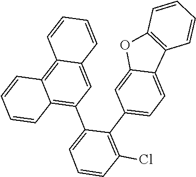 [SMILES: Clc1cccc(-c2cc3ccccc3c3ccccc23)c1-c1ccc2c(c1)oc1ccccc12] IM-B8 | 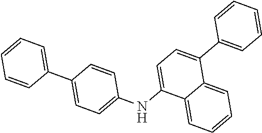 [SMILES: c1ccc(-c2ccc(Nc3ccc(-c4ccccc4)c4ccccc34)cc2)cc1] IM-N8 | 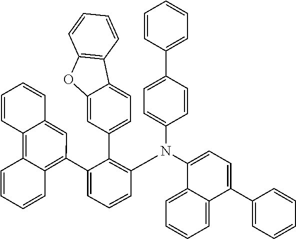 [SMILES: c1ccc(-c2ccc(N(c3cccc(-c4cc5ccccc5c5ccccc45)c3-c3ccc4c(c3)oc3ccccc34)c3ccc(-c4ccccc4)c4ccccc34)cc2)cc1] 580 | 42.5 | 790.3 |
| 45 | 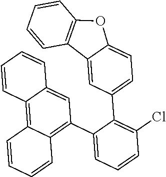 [SMILES: Clc1cccc(-c2cc3ccccc3c3ccccc23)c1-c1ccc2oc3ccccc3c2c1] IM-B9 | 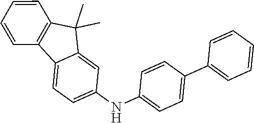 [SMILES: CC1(C)c2ccccc2-c2ccc(Nc3ccc(-c4ccccc4)cc3)cc21] CAS: 897671-69-1 | 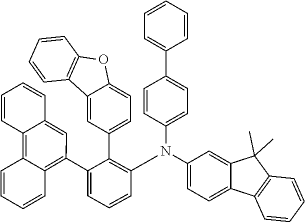 [SMILES: CC1(C)c2ccccc2-c2ccc(N(c3ccc(-c4ccccc4)cc3)c3cccc(-c4cc5ccccc5c5ccccc45)c3-c3ccc4oc5ccccc5c4c3)cc21] 535 | 49.0 | 780.3 |
| 46 | 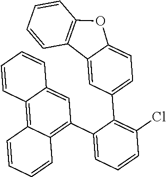 [SMILES: Clc1cccc(-c2cc3ccccc3c3ccccc23)c1-c1ccc2oc3ccccc3c2c1] IM-B9 | 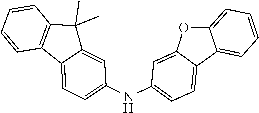 [SMILES: CC1(C)c2ccccc2-c2ccc(Nc3ccc4c(c3)oc3ccccc34)cc21] CAS: 1427556-50-0 | 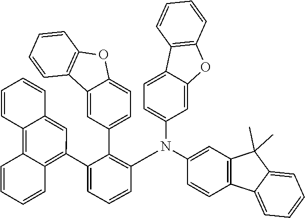 [SMILES: CC1(C)c2ccccc2-c2ccc(N(c3ccc4c(c3)oc3ccccc34)c3cccc(-c4cc5ccccc5c5ccccc45)c3-c3ccc4oc5ccccc5c4c3)cc21] 545 | 44.3 | 794.3 |
| 47 | 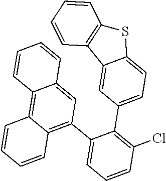 [SMILES: Clc1cccc(-c2cc3ccccc3c3ccccc23)c1-c1ccc2sc3ccccc3c2c1] IM-B10 | 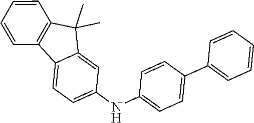 [SMILES: CC1(C)c2ccccc2-c2ccc(Nc3ccc(-c4ccccc4)cc3)cc21] CAS: 897671-69-1 | 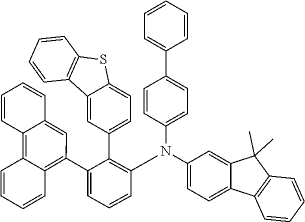 [SMILES: CC1(C)c2ccccc2-c2ccc(N(c3ccc(-c4ccccc4)cc3)c3cccc(-c4cc5ccccc5c5ccccc45)c3-c3ccc4sc5ccccc5c4c3)cc21] 569 | 47.2 | 796.3 |
| 48 | 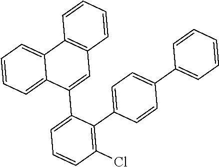 [SMILES: Clc1cccc(-c2cc3ccccc3c3ccccc23)c1-c1ccc(-c2ccccc2)cc1] IM-B3 | 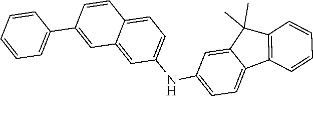 IM-N9 | 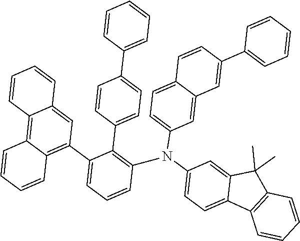 [SMILES: CC1(C)c2ccccc2-c2ccc(N(c3ccc4ccc(-c5ccccc5)cc4c3)c3cccc(-c4cc5ccccc5c5ccccc45)c3-c3ccc(-c4ccccc4)cc3)cc21] 577 | 36.2 | 816.4 |
| 49 | 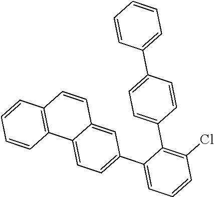 [SMILES: Clc1cccc(-c2ccc3c(ccc4ccccc43)c2)c1-c1ccc(-c2ccccc2)cc1] IM-B12 | 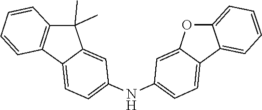 [SMILES: CC1(C)c2ccccc2-c2ccc(Nc3ccc4c(c3)oc3ccccc34)cc21] CAS: 1427556-50-0 | 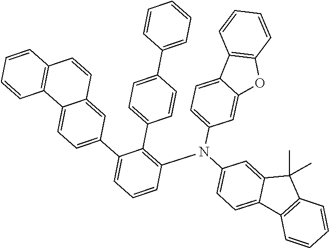 419 | 41.7 | 780.3 |
| 50 | 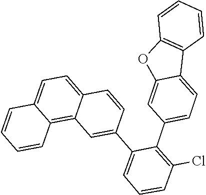 [SMILES: Clc1cccc(-c2ccc3ccc4ccccc4c3c2)c1-c1ccc2c(c1)oc1ccccc12] IM-B13 | 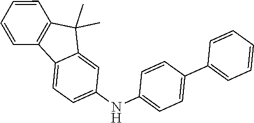 [SMILES: CC1(C)c2ccccc2-c2ccc(Nc3ccc(-c4ccccc4)cc3)cc21] CAS: 897671-69-1 | 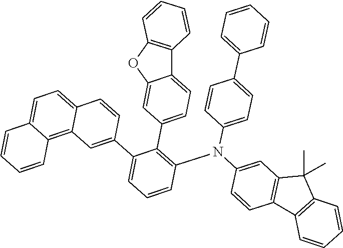 525 | 45.0 | 780.3 |
| 51 | 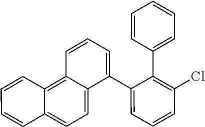 [SMILES: Clc1cccc(-c2cccc3c2ccc2ccccc23)c1-c1ccccc1] IM-B14 | 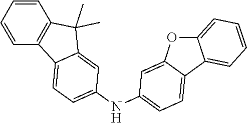 [SMILES: CC1(C)c2ccccc2-c2ccc(Nc3ccc4c(c3)oc3ccccc34)cc21] CAS: 1427556-50-0 | 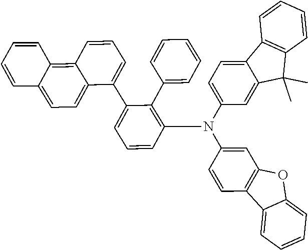 572 | 45.1 | 704.3 |

NMR data of some Compounds are shown in Table 5:

TABLE 5

| Compound | ¹H-NMR (400 MHz, CD₂Cl₂) δ: ppm |
|---|---|
| Compound 15 | 8.47-8.38 (m, 2H), 7.63-7.54 (m, 3H), 7.48-7.41 (m, 3H), 7.39-7.30 (m, 6H), |
| | 7.30-7.13 (m, 10H), 7.10 (d, 2H), 6.95-6.89 (m, 3H), 6.71 (d, 2H), 6.65-6.61 (m, |
| | 2H), 1.11 (s, 3H), 1.06 (s, 3H). |
| Compound 358 | 8.57 (t, 2H), 7.73-7.68 (m, 2H), 7.60-7.45 (m, 12H), 7.44-7.36 (m, 6H), 7.36-7.32 |
| | (m, 2H), 7.30-7.17 (m, 3H), 7.06 (d, 2H), 6.83 (s, 1H), 6.77 (d, 1H), 6.69-6.55 (m, |
| | 2H), 1.29 (s, 3H), 1.26 (s, 3H). |
| Compound 408 | 8.65-8.56 (m, 2H), 7.87 (d, 1H), 7.82-7.67 (m, 3H), 7.67-7.23 (m, 16H), 7.22-7.08 |
| | (m, 7H), 7.04 (d, 1H), 6.96 (s, 1H), 6.94-6.68 (m, 4H), 1.30 (s, 3H), 1.24 (s, 3H). |
| Compound 510 | 8.58-8.51 (m, 2H), 7.72 (d, 2H), 7.63-7.44 (m, 12H), 7.41-7.32 (m, 6H), 7.28-7.13 |
| | (m, 7H), 7.12-7.02 (m, 4H), 6.84 (d, 2H), 1.06 (s, 6H). |

### Fabrication and Evaluation of Organic Electroluminescent Device

### Example 1: Red Organic Electroluminescent Device

The anode was prepared through the following process: an ITO substrate with an ITO thickness of 1300 Å was cut into dimensions of 40 mm (length)×40 mm (width)×0.7 mm (thickness) and prepared into an experimental substrate having anode and insulation layer patterns with photolithography process. The surface treatment was performed using ultraviolet ozone and O₂:N₂ plasma to increase the work function of the anode, and the surface of the ITO substrate was cleaned using organic solvents to remove impurities and oil stains.

On the experimental substrate (anode), Compound HAT-CN was deposited by vacuum evaporation to form a hole injection layer with a thickness of 100 Å.

On the hole injection layer, Compound HT-1 was deposited by vacuum evaporation to form a first hole transport layer with a thickness of 1200 Å.

On the first hole transport, Compound 11 was deposited by vacuum evaporation to form a second hole transport layer with a thickness of 800 Å.

On the second hole transport layer, Compound RH-1 and Compound RD-1 were deposited at a deposition rate ratio of 95%:5% to form an organic light-emitting layer with a thickness of 300 Å.

On the organic light-emitting layer, Compound HB-1 was deposited by vacuum evaporation to form a hole blocking layer with a thickness of 50 Å.

On the hole blocking layer, Compound ET-1 and LiQ were deposited at a deposition rate ratio of 1:1 to form an electron transport layer with a thickness of 300 Å. Yb was deposited on the electron transport layer to form an electron injection layer (EIL) with a thickness of 15 Å. Then, magnesium (Mg) and silver (Ag) were deposited on the electron injection layer at a deposition rate of 1:9 to form a cathode with a thickness of 130 Å.

In addition, Compound CP-1 was deposited by vacuum evaporation on the above cathode to form an organic protection layer (CPL) with a thickness of 650 Å, thus completing the fabrication of the red organic electroluminescent device.

### Example 2 to Example 51

Organic electroluminescent devices were fabricated using the same method as in Example 1, except that the compounds listed in Table 7 were respectively used in the formation of the second hole transport layer.

### Comparative Example 1 to Comparative Example 5

In Comparative Example 1 to Comparative Example 5, organic electroluminescent devices were fabricated using the same method as in Example 1, except that the Compound A to Compound E were respectively used instead of Compound 11.

The main material structures used in the above Example and Comparative Example are shown in Table 6 below.

TABLE 6 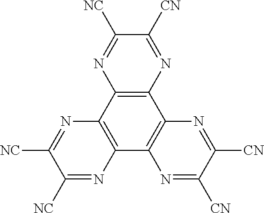 [SMILES: N#Cc1nc2c3nc(C#N)c(C#N)nc3c3nc(C#N)c(C#N)nc3c2nc1C#N]
HAT-CN 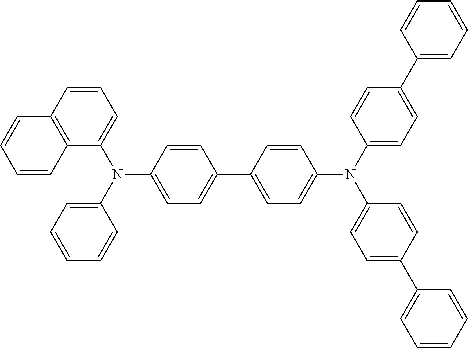 [SMILES: c1ccc(-c2ccc(N(c3ccc(-c4ccccc4)cc3)c3ccc(-c4ccc(N(c5ccccc5)c5cccc6ccccc56)cc4)cc3)cc2)cc1]
HT-1 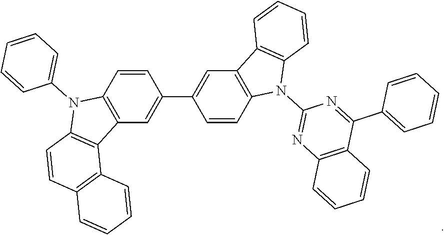 [SMILES: c1ccc(-c2nc(-n3c4ccccc4c4cc(-c5ccc6c(c5)c5c7ccccc7ccc5n6-c5ccccc5)ccc43)nc3ccccc23)cc1]
RH-1 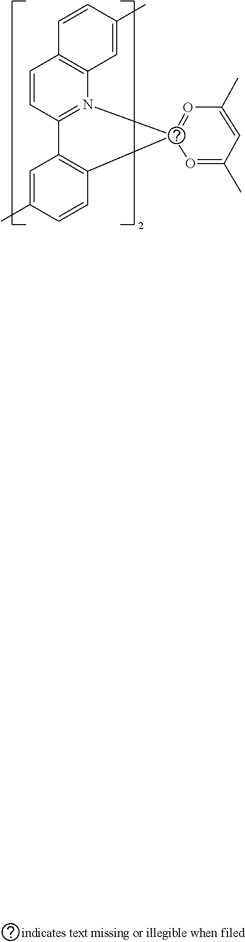
RD-1 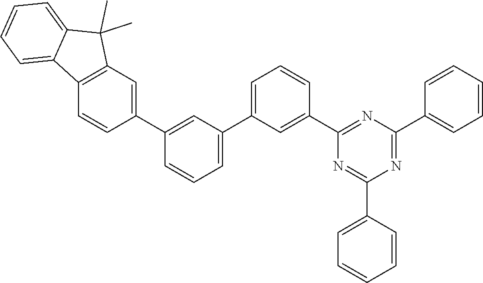 [SMILES: CC1(C)c2ccccc2-c2ccc(-c3cccc(-c4cccc(-c5nc(-c6ccccc6)nc(-c6ccccc6)n5)c4)c3)cc21]
HB-1 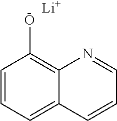 [SMILES: [Li+] ; Oc1cccc2cccnc12]
LiQ 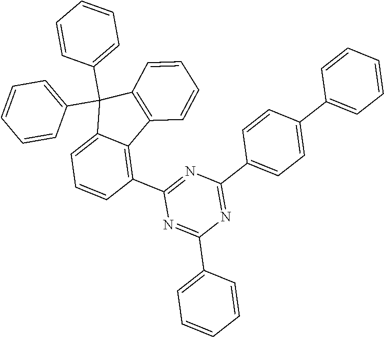
ET-1 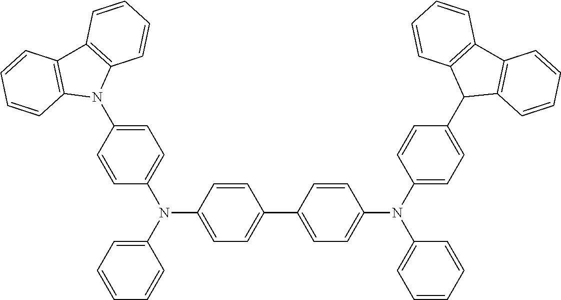 [SMILES: c1ccc(N(c2ccc(-c3ccc(N(c4ccccc4)c4ccc(-n5c6ccccc6c6ccccc65)cc4)cc3)cc2)c2ccc(C3c4ccccc4-c4ccccc43)cc2)cc1]
CP-1 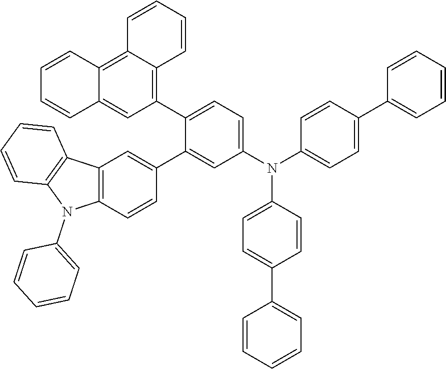 [SMILES: c1ccc(-c2ccc(N(c3ccc(-c4ccccc4)cc3)c3ccc(-c4cc5ccccc5c5ccccc45)c(-c4ccc5c(c4)c4ccccc4n5-c4ccccc4)c3)cc2)cc1]
Compound A 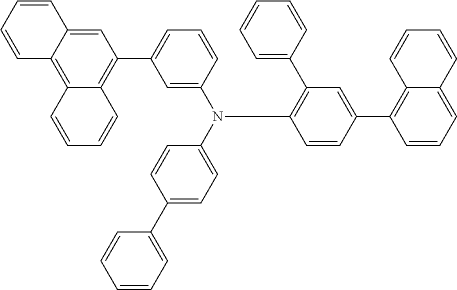 [SMILES: c1ccc(-c2ccc(N(c3cccc(-c4cc5ccccc5c5ccccc45)c3)c3ccc(-c4cccc5ccccc45)cc3-c3ccccc3)cc2)cc1]
Compound B 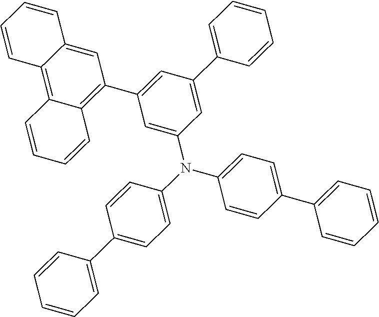 [SMILES: c1ccc(-c2ccc(N(c3ccc(-c4ccccc4)cc3)c3cc(-c4ccccc4)cc(-c4cc5ccccc5c5ccccc45)c3)cc2)cc1]
Compound C 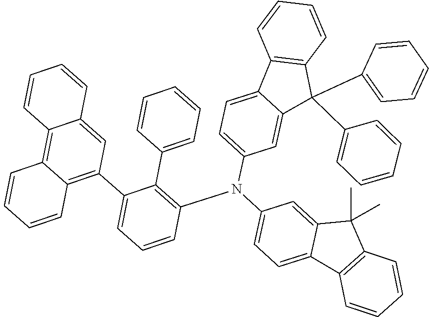
Compound D 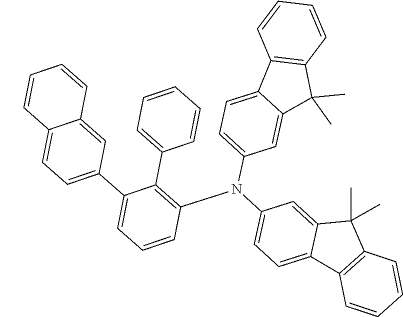
Compound E

Performances of the devices prepared according to each of Examples and Comparative examples were tested, and the IVL (Operating voltage, Luminous efficiency, and Color coordinates) data were measured at a current density of 10 mA/cm². The T₉₅ lifespan of the devices was tested under a constant current density of 20 mA/cm². Performances of the devices are shown in Table 7.

TABLE 7
Performance test results of red organic electroluminescent device

| | | Operating | Luminous | T₉₅ device | Color |
|---|---|---|---|---|---|
| | Second Hole | voltage | efficiency | lifespan | coordinates |
| Example | Transport Layer | (V) | (Cd/A) | (h) | CIEx, CIEy |
| Example 1 | Compound 11 | 3.92 | 44.5 | 333 | 0.682, 0.321 |
| Example 2 | Compound 15 | 3.89 | 44.6 | 337 | 0.683, 0.322 |
| Example 3 | Compound 20 | 3.87 | 43.2 | 325 | 0.682, 0.321 |
| Example 4 | Compound 43 | 3.89 | 44.5 | 327 | 0.682, 0.321 |
| Example 5 | Compound 53 | 3.93 | 44.4 | 329 | 0.683, 0.322 |
| Example 6 | Compound 65 | 3.82 | 44.2 | 326 | 0.682, 0.321 |
| Example 7 | Compound 79 | 3.90 | 43.5 | 327 | 0.682, 0.321 |
| Example 8 | Compound 100 | 3.85 | 45.2 | 336 | 0.681, 0.320 |
| Example 9 | Compound 130 | 3.85 | 44.8 | 334 | 0.681, 0.320 |
| Example 10 | Compound 141 | 3.92 | 45.2 | 321 | 0.682, 0.322 |
| Example 11 | Compound 160 | 3.87 | 43.5 | 326 | 0.683, 0.321 |
| Example 12 | Compound 173 | 3.91 | 43.3 | 324 | 0.681, 0.320 |
| Example 13 | Compound 190 | 3.88 | 43.7 | 341 | 0.683, 0.322 |
| Example 14 | Compound 201 | 3.93 | 44.4 | 328 | 0.683, 0.323 |
| Example 15 | Compound 213 | 3.91 | 44.9 | 337 | 0.683, 0.322 |
| Example 16 | Compound 222 | 3.84 | 43.7 | 324 | 0.682, 0.323 |
| Example 17 | Compound 236 | 3.82 | 45.5 | 339 | 0.681, 0.320 |
| Example 18 | Compound 250 | 3.94 | 45.4 | 331 | 0.683, 0.323 |
| Example 19 | Compound 253 | 3.85 | 44.3 | 332 | 0.683, 0.321 |
| Example 20 | Compound 267 | 3.83 | 44.7 | 322 | 0.682, 0.321 |
| Example 21 | Compound 310 | 3.92 | 44.4 | 337 | 0.683, 0.323 |
| Example 22 | Compound 330 | 3.91 | 45.3 | 340 | 0.681, 0.322 |
| Example 23 | Compound 321 | 3.94 | 46.8 | 385 | 0.682, 0.322 |
| Example 24 | Compound 345 | 3.93 | 47.1 | 380 | 0.681, 0.322 |
| Example 25 | Compound 358 | 3.89 | 47.0 | 398 | 0.683, 0.322 |
| Example 26 | Compound 395 | 3.84 | 49.7 | 411 | 0.681, 0.322 |
| Example 27 | Compound 360 | 3.80 | 48.4 | 409 | 0.681, 0.322 |
| Example 28 | Compound 363 | 3.86 | 46.3 | 385 | 0.681, 0.320 |
| Example 29 | Compound 380 | 3.78 | 48.5 | 412 | 0.682, 0.323 |
| Example 30 | Compound 381 | 3.89 | 46.9 | 388 | 0.682, 0.321 |
| Example 31 | Compound 576 | 3.87 | 46.6 | 390 | 0.682, 0.323 |
| Example 32 | Compound 386 | 3.85 | 47.6 | 379 | 0.682, 0.322 |
| Example 33 | Compound 391 | 3.90 | 46.3 | 378 | 0.681, 0.322 |
| Example 34 | Compound 417 | 3.88 | 46.6 | 395 | 0.683, 0.322 |
| Example 35 | Compound 398 | 3.82 | 47.1 | 387 | 0.683, 0.322 |
| Example 36 | Compound 408 | 3.90 | 46.8 | 382 | 0.682, 0.323 |
| Example 37 | Compound 432 | 3.91 | 46.5 | 381 | 0.682, 0.321 |
| Example 38 | Compound 450 | 3.88 | 46.2 | 380 | 0.683, 0.323 |
| Example 39 | Compound 460 | 3.92 | 47.5 | 385 | 0.683, 0.321 |
| Example 40 | Compound 485 | 3.95 | 47.1 | 389 | 0.682, 0.321 |
| Example 41 | Compound 506 | 3.91 | 45.4 | 384 | 0.683, 0.323 |
| Example 42 | Compound 510 | 3.86 | 47.4 | 383 | 0.681, 0.322 |
| Example 43 | Compound 535 | 3.93 | 46.5 | 389 | 0.683, 0.322 |
| Example 44 | Compound 545 | 3.90 | 46.2 | 395 | 0.683, 0.322 |
| Example 45 | Compound 288 | 3.81 | 47.2 | 410 | 0.683, 0.323 |
| Example 46 | Compound 569 | 3.94 | 46.8 | 379 | 0.681, 0.322 |
| Example 47 | Compound 577 | 3.95 | 46.3 | 379 | 0.683, 0.322 |
| Example 48 | Compound 580 | 3.79 | 48.3 | 396 | 0.682, 0.321 |
| Example 49 | Compound 419 | 4.22 | 46.0 | 377 | 0.682, 0.323 |
| Example 50 | Compound 525 | 4.18 | 46.2 | 375 | 0.682, 0.321 |
| Example 51 | Compound 572 | 4.21 | 46.3 | 374 | 0.682, 0.321 |
| Comparative | Compound A | 4.34 | 35.9 | 240 | 0.683, 0.322 |
| example 1 | | | | | |
| Comparative | Compound B | 4.49 | 35.3 | 258 | 0.683, 0.323 |
| example 2 | | | | | |
| Comparative | Compound C | 4.42 | 36.7 | 265 | 0.681, 0.320 |
| example 3 | | | | | |
| Comparative | Compound D | 4.38 | 38.0 | 278 | 0.683, 0.322 |
| example 4 | | | | | |
| Comparative | Compound E | 4.36 | 38.7 | 271 | 0.683, 0.323 |
| example 5 | | | | | |

As can be seen from Table 7, the red organic electroluminescent devices fabricated in Example 1 to Example 51 in which the organic compounds of the present application were used as the second hole transport layer, have characteristics of low operating voltages, high efficiency, and long lifespans. Compared to Comparative Example 1 to Comparative Example 5, the voltage was reduced by at least 0.12V, the efficiency was increased by at least 11.6%, and the lifespan was prolonged by at least 15.5%. It can be seen that the utilization of the organic compound of the present application as the second hole transport layer in organic electroluminescent devices can significantly enhance the luminous efficiency and T₉₅ lifespan of the organic electroluminescent devices, while significantly reducing the driving voltage and improving the luminous efficiency.

The organic compound of the present application is centered around a benzene ring, in which an aromatic amine is attached to the central benzene ring, and a phenanthrene group and other aromatic groups are attached to the ortho- and meta-positions of the benzene ring. The consecutive adjacent substituents on the central benzene ring improve the spatial distortion of the molecule, which can elevate the glass transition temperature of the material, ensuring the formation of a stable amorphous thin film during vapor deposition, thereby prolonging the lifespan of the device. The indirect linkage of the phenanthryl with the aromatic amino through the benzene ring further effectively improves the hole mobility of the molecule and reduces the potential barrier for carriers to be injected into the organic light-emitting layer, thereby lowering the voltage of the device, and enhancing the luminous efficiency of the device. Furthermore, in the compounds of the present application, one of the substituents on the aromatic amine is defined as a specific group, which enhances the thermal stability of the molecule. The compounds of the present application can avoid thermal stability problem during the fabrication and production of devices, rendering them more suitable for industrial applications.

The preferred embodiments of the present application are described in detail above. However, the present application is not limited to the specific details of the above embodiments. Within the scope of the technical concept of the present application, various simple modifications can be made to the technical solutions of the present application, and all of these simple modifications fall within the protection scope of the present application.

1. An organic compound, having a structure shown in a Formula 1: 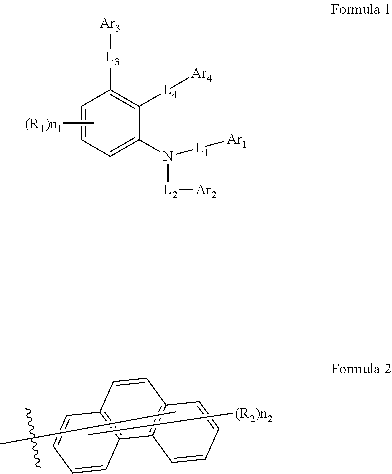
wherein, Ar₁ is selected from a substituted or unsubstituted aryl having 6 to 12 carbon atoms, or a substituted or unsubstituted heteroaryl having 6 to 18 carbon atoms;
Ar₂, Ar₃ and Ar₄ are the same or different, and are each independently selected from a substituted or unsubstituted aryl having 6 to 40 carbon atoms, or a substituted or unsubstituted heteroaryl having 3 to 40 carbon atoms, and one or two of Ar₃ and Ar₄ are selected from the group represented by a Formula 2;
L₁ is selected from a single bond, or a substituted or unsubstituted arylene having 6 to 12 carbon atoms;
L₂, L₃ and L₄ are the same or different, and are each independently selected from a single bond, a substituted or unsubstituted arylene having 6 to 30 carbon atoms, or a substituted or unsubstituted heteroarylene having 3 to 30 carbon atoms;
R₁ and R₂ are the same or different, and each independently selected from a deuterium, a halogen group, a cyano, an alkyl having 1 to 5 carbon atoms, or an aryl having 6 to 12 carbon atoms; or any two adjacent R₁ are interconnected to form a ring, or any two adjacent R₂ are interconnected to form a ring;
n₁ is the number of R₁, n₁ is selected from 0, 1, 2, or 3; when n₁ is greater than 1, any two R₁ are the same or different;
n₂ is the number of R₂, n₂ is selected from 0, 1, 2, 3, 4, 5, 6, 7, 8, or 9; when n₂ is greater than 1, any two R₂ are the same or different;
the substituent(s) of Ar₁ and L₁ are the same or different, and are each independently selected from a deuterium, a halogen group, a cyano, an alkyl having 1 to 10 carbon atoms, or an aryl having 6 to 12 carbon atoms;
the substituent(s) of Ar₂, Ar₃, Ar₄, L₂, L₃ and L₄ are the same or different, and each independently selected from a deuterium, a halogen group, a cyano, an alkyl having 1 to 10 carbon atoms, an aryl having 6 to 20 carbon atoms, a deuterated aryl having 6 to 20 carbon atoms, a haloaryl having 6 to 20 carbon atoms, or a heteroaryl having 3 to 20 carbon atoms; optionally, any two adjacent substituents of Ar₁ form a saturated or unsaturated 3-membered to 15-membered ring; optionally, any two adjacent substituents of Ar₂ form a saturated or unsaturated 3-membered to 15-membered ring.

2. The organic compound according to claim 1, wherein the Formula 1 is selected from structure represented by a Formula I-1, a Formula I-2, a Formula I-3, a Formula I-4, a Formula I-5, a Formula I-6, a Formula I-7, a Formula I-8, or a Formula I-9:

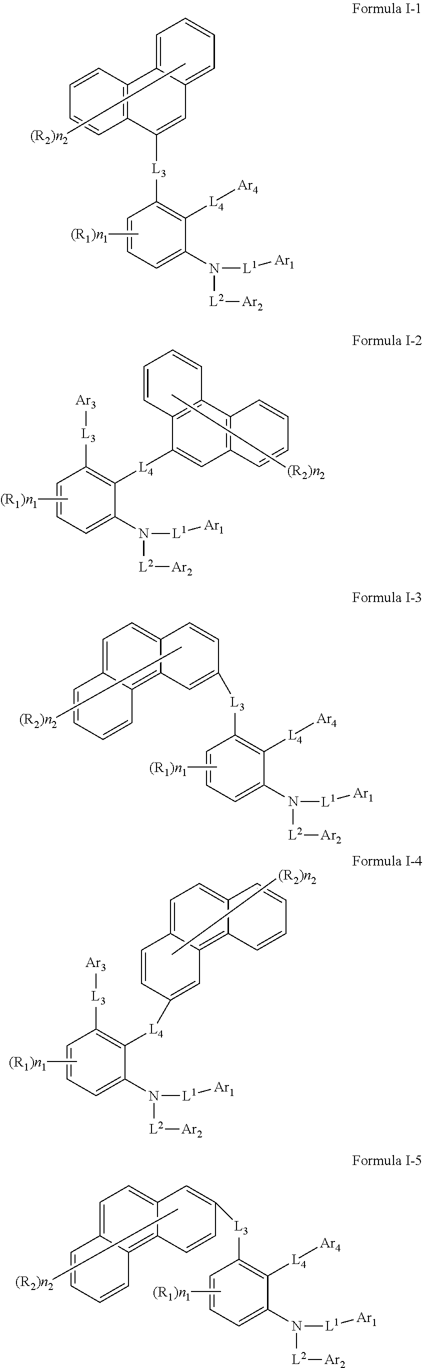

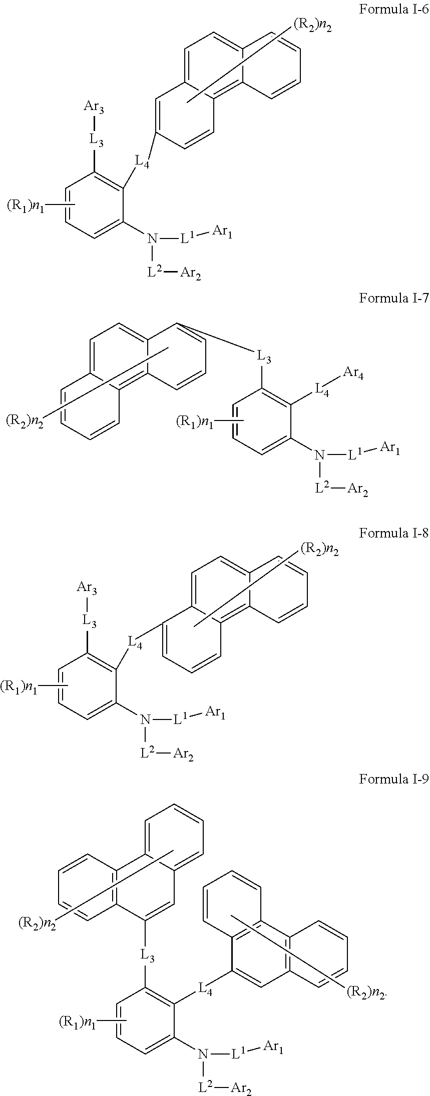

3. The organic compound according to claim 1, wherein Ar₁ is selected from a substituted or unsubstituted phenyl, a substituted or unsubstituted naphthyl, a substituted or unsubstituted biphenyl, a substituted or unsubstituted dibenzofuranyl, a substituted or unsubstituted dibenzothienyl, or a substituted or unsubstituted carbazolyl;
optionally, the substituent(s) of Ar₁ are each independently selected from a deuterium, a fluorine, a cyano, a methyl, an ethyl, an isopropyl, a tert-butyl, or a phenyl.

4. The organic compound according to claim 1, wherein Ar₃ and Ar₄ are the same or different, and are each independently selected from a substituted or unsubstituted aryl having 6 to 25 carbon atoms, or a substituted or unsubstituted heteroaryl having 12 to 25 carbon atoms, and one or two of Ar₃ and Ar₄ are selected from the group represented by the Formula 2;
optionally, the substituent(s) of Ar₃ and Ar₄ are each independently selected from a deuterium, a fluorine, a cyano, an alkyl having 1 to 5 carbon atoms, a trimethylsilyl, a trifluoromethyl, an aryl having 6 to 12 carbon atoms, a deuterated aryl having 6 to 12 carbon atoms, a haloaryl having 6 to 12 carbon atoms, or a heteroaryl having 12 to 18 carbon atoms.

5. The organic compound according to claim 1, wherein Ar₂ is selected from a substituted or unsubstituted group W, wherein the unsubstituted group W is selected from the following groups: 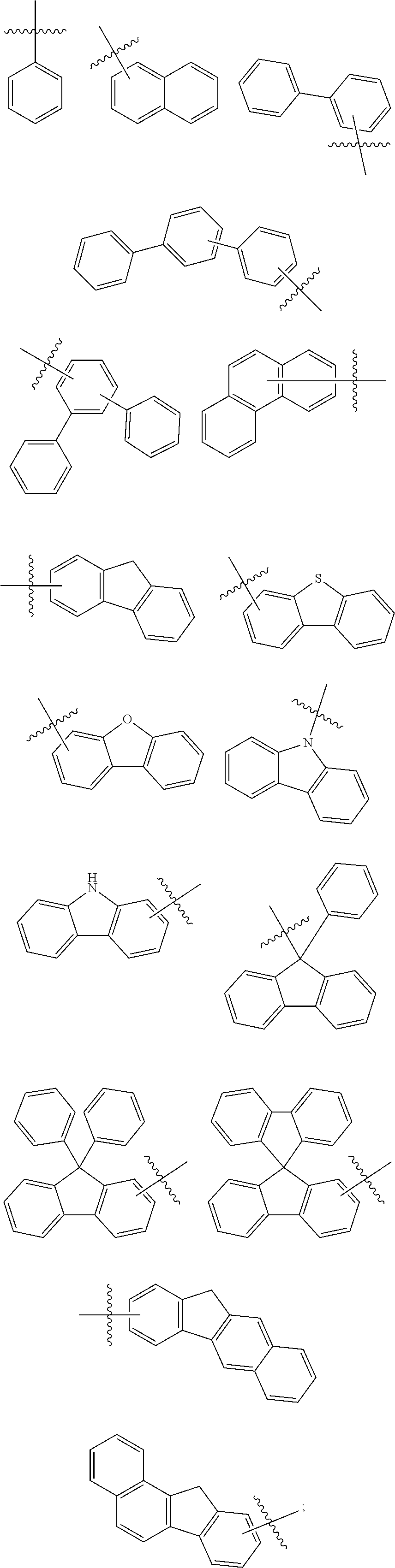
wherein, the substituted group W has one or more substituent(s), and the substituents are each independently selected from a deuterium, a fluorine, a cyano, a methyl, an ethyl, an isopropyl, a tert-butyl, a trideuterated methyl, a trimethylsilyl, a trifluoromethyl, a phenyl, a naphthyl, a biphenyl, a dibenzofuranyl, or a dibenzothienyl, and when the number of the substituents is greater than 1, the substituents are each the same or different.

6. The organic compound according to claim 1, wherein Ar₃ and Ar₄ are the same or different, and are each independently selected from a substituted or unsubstituted phenyl, a substituted or unsubstituted naphthyl, a substituted or unsubstituted biphenyl, a substituted or unsubstituted fluorenyl, a substituted or unsubstituted terphenyl, a substituted or unsubstituted spirobifluorenyl, a substituted or unsubstituted dibenzofuranyl, a substituted or unsubstituted dibenzothienyl, a substituted or unsubstituted carbazolyl, or the group represented by the Formula 2, and one or two of Ar₃ and Ar₄ are selected from the groups represented by the Formula 2;
optionally, the substituent(s) of Ar₃ and Ar₄ are each independently selected from a deuterium, a fluorine, a cyano, a methyl, an ethyl, an isopropyl, a tert-butyl, a trideuterated methyl, a trimethylsilyl, a trifluoromethyl, a phenyl, a naphthyl, a biphenyl, a dibenzofuranyl, or a dibenzothienyl.

7. The organic compound according to claim 1, wherein 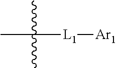
is selected from the group consisting of the following groups:

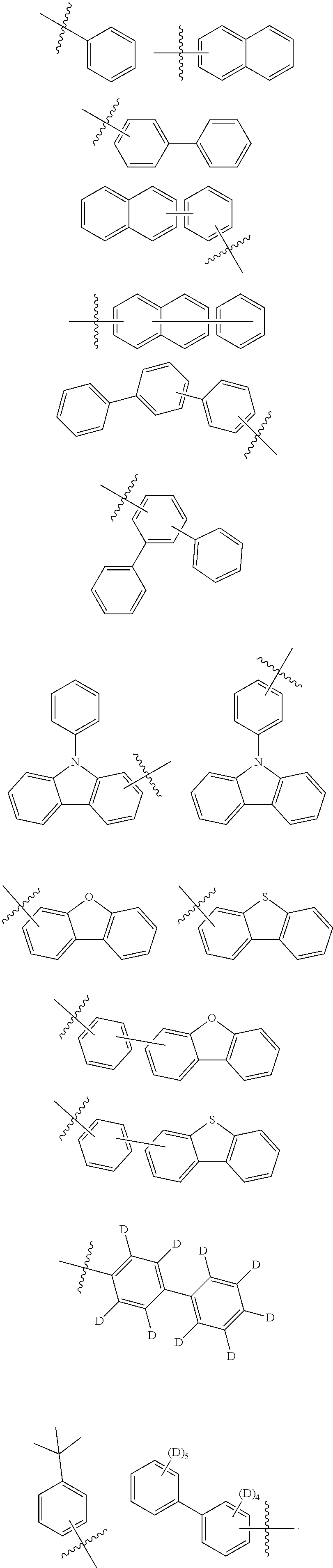

8. The organic compound according to claim 1, wherein Ar₄ is the group represented by Formula 2 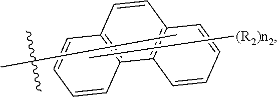
and Ar₃ is selected from the following groups: 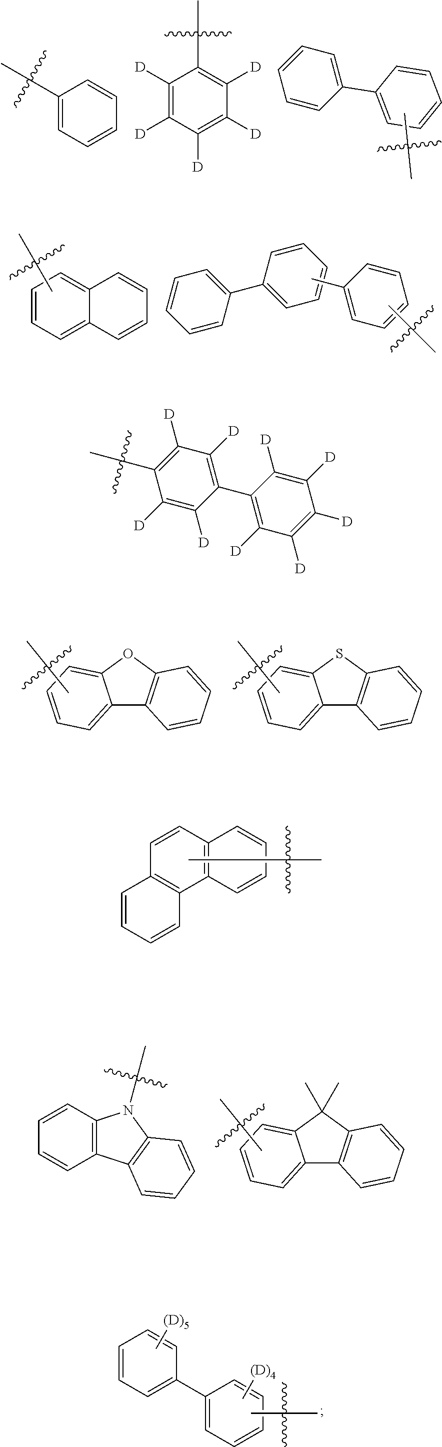
or Ar₃ is the group represented by Formula 2 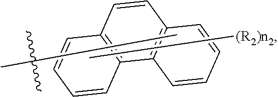
and Ar₄ is selected from the following groups:

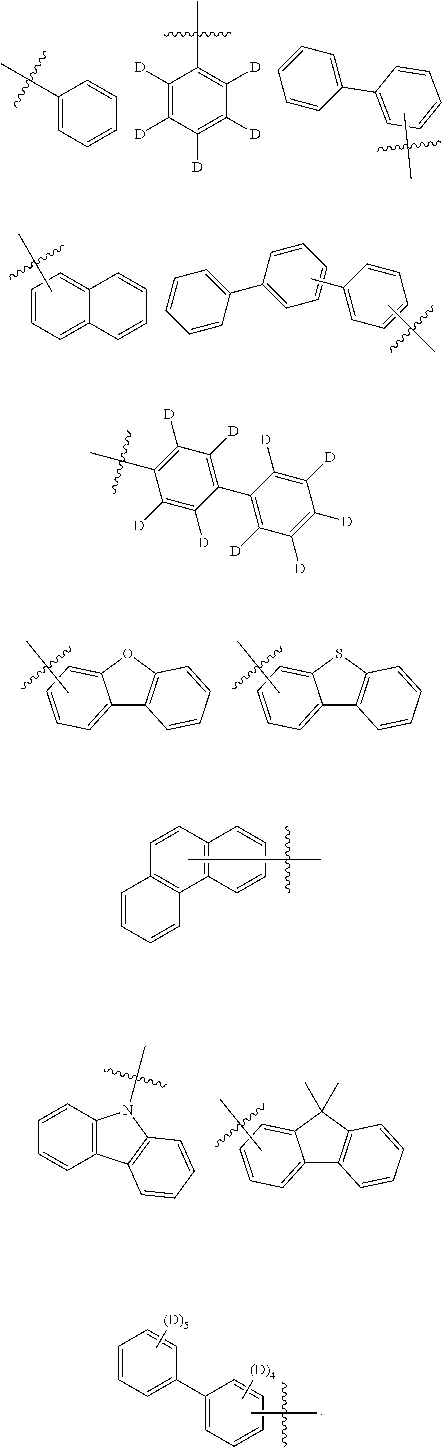

9. The organic compound according to claim 1, wherein L₁ is selected from a single bond, a substituted or unsubstituted phenylene, a substituted or unsubstituted naphthylene, or a substituted or unsubstituted biphenylene;
optionally, the substituent(s) of L₁ are each independently selected from a deuterium, a fluorine, a cyano, a methyl, an ethyl, an isopropyl, a tert-butyl, or a phenyl.

10. The organic compound according to claim 1, wherein Formula 2 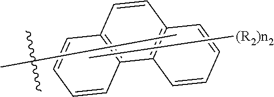
is selected from the following groups:

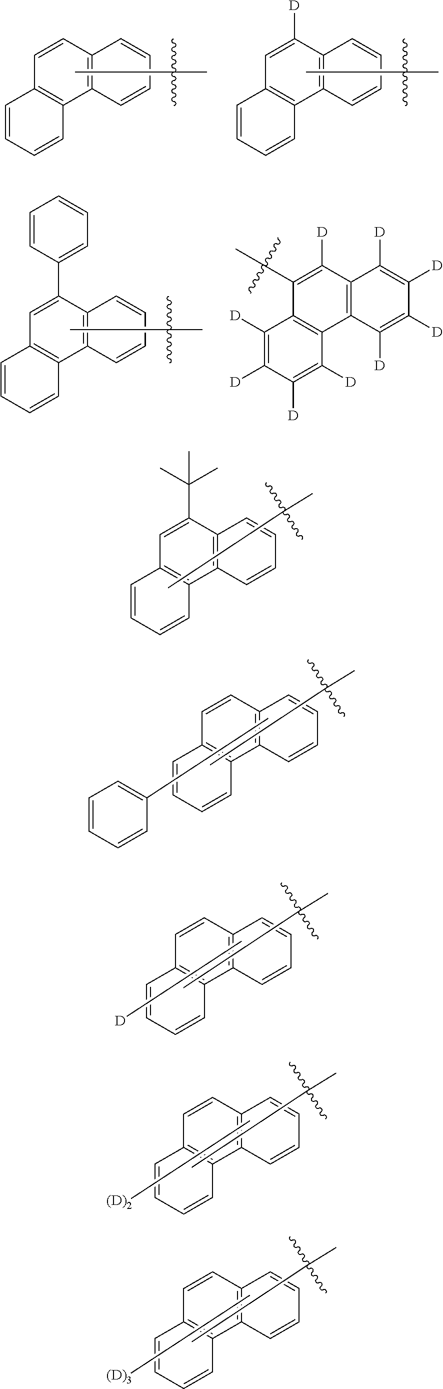

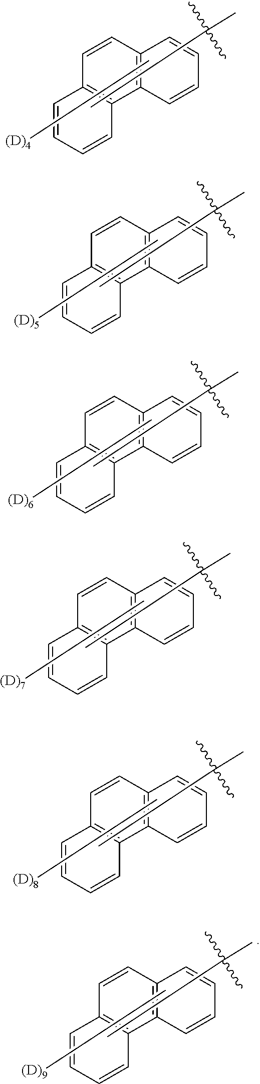

11. The organic compound according to claim 1, wherein L₂, L₃, and L₄ are the same or different, and are each independently selected from a single bond, a substituted or unsubstituted phenylene, a substituted or unsubstituted naphthylene, a substituted or unsubstituted biphenylene, a substituted or unsubstituted fluorenylene, a substituted or unsubstituted dibenzofuranylene, a substituted or unsubstituted dibenzothienylene, or a substituted or unsubstituted carbazolylene;
optionally, the substituent(s) of L₂, L₃ and L₄ are each independently selected from a deuterium, a fluorine, a cyano, a methyl, an ethyl, an isopropyl, a tert-butyl, a phenyl, or a naphthyl.

12. The organic compound according to claim 1, wherein R₁ and R₂ are the same or different, and are each independently selected from a deuterium, a fluorine, a cyano, a methyl, an ethyl, an isopropyl, a tert-butyl, a phenyl, a trimethylsilyl, or a phenyl.

13. The organic compound according to claim 1, wherein the organic compound is selected from the group consisting of the following compounds:

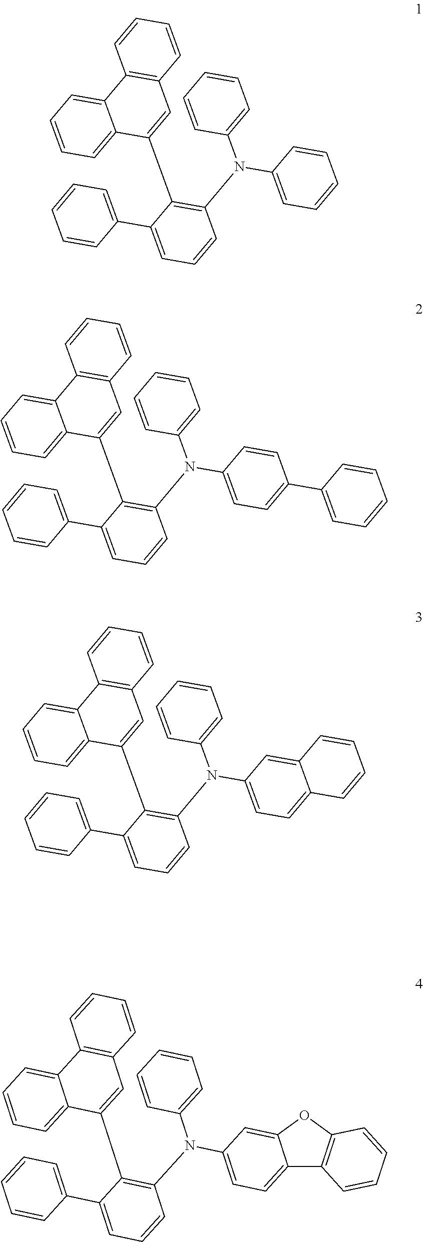 [SMILES: c1ccc(-c2cccc(N(c3ccccc3)c3ccccc3)c2-c2cc3ccccc3c3ccccc23)cc1 ; c1ccc(-c2ccc(N(c3ccccc3)c3cccc(-c4ccccc4)c3-c3cc4ccccc4c4ccccc34)cc2)cc1 ; c1ccc(-c2cccc(N(c3ccccc3)c3ccc4ccccc4c3)c2-c2cc3ccccc3c3ccccc23)cc1 ; c1ccc(-c2cccc(N(c3ccccc3)c3ccc4c(c3)oc3ccccc34)c2-c2cc3ccccc3c3ccccc23)cc1]

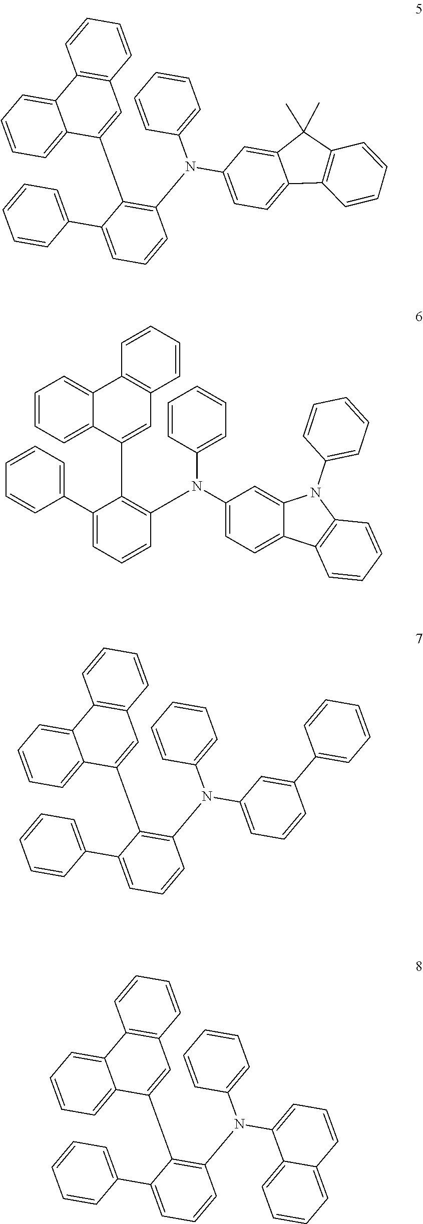 [SMILES: CC1(C)c2ccccc2-c2ccc(N(c3ccccc3)c3cccc(-c4ccccc4)c3-c3cc4ccccc4c4ccccc34)cc21 ; c1ccc(-c2cccc(N(c3ccccc3)c3ccc4c5ccccc5n(-c5ccccc5)c4c3)c2-c2cc3ccccc3c3ccccc23)cc1 ; c1ccc(-c2cccc(N(c3ccccc3)c3cccc(-c4ccccc4)c3-c3cc4ccccc4c4ccccc34)c2)cc1 ; c1ccc(-c2cccc(N(c3ccccc3)c3cccc4ccccc34)c2-c2cc3ccccc3c3ccccc23)cc1]

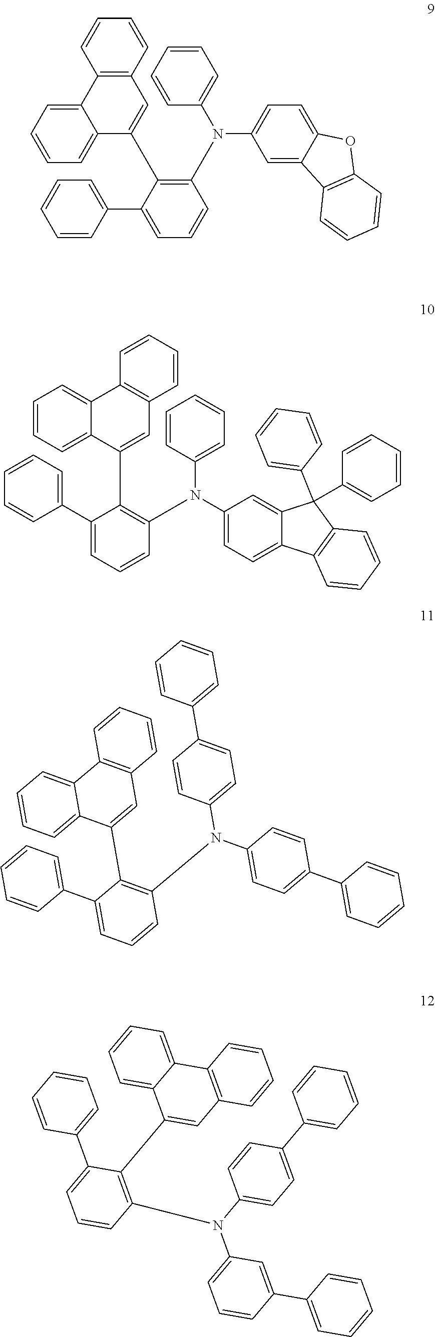 [SMILES: c1ccc(-c2cccc(N(c3ccccc3)c3ccc4oc5ccccc5c4c3)c2-c2cc3ccccc3c3ccccc23)cc1 ; c1ccc(-c2cccc(N(c3ccccc3)c3ccc4c(c3)C(c3ccccc3)(c3ccccc3)c3ccccc3-4)c2-c2cc3ccccc3c3ccccc23)cc1 ; c1ccc(-c2ccc(N(c3ccc(-c4ccccc4)cc3)c3cccc(-c4ccccc4)c3-c3cc4ccccc4c4ccccc34)cc2)cc1 ; c1ccc(-c2ccc(N(c3cccc(-c4ccccc4)c3)c3cccc(-c4ccccc4)c3-c3cc4ccccc4c4ccccc34)cc2)cc1]

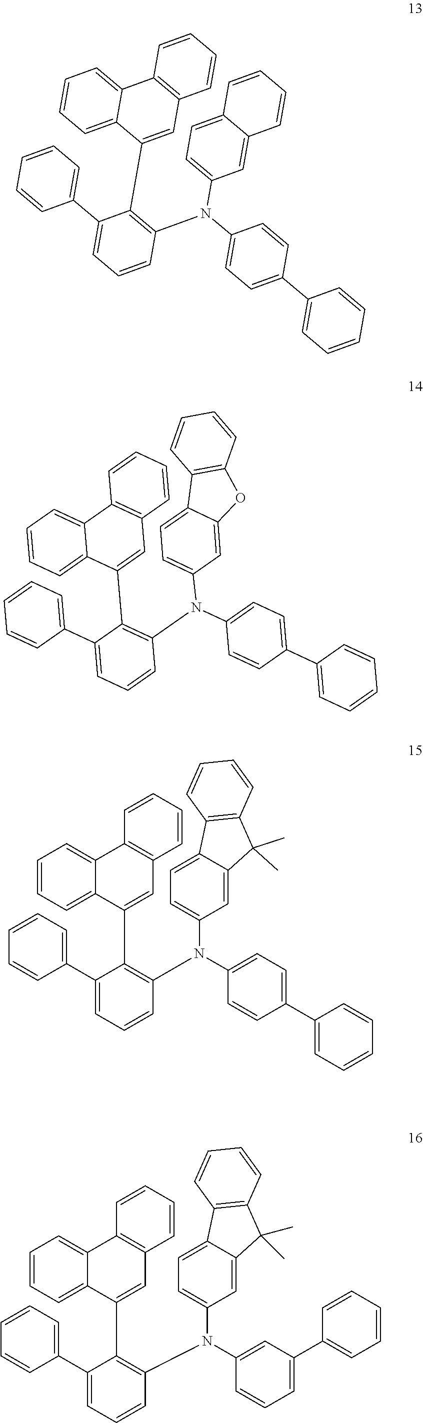 [SMILES: c1ccc(-c2ccc(N(c3ccc4ccccc4c3)c3cccc(-c4ccccc4)c3-c3cc4ccccc4c4ccccc34)cc2)cc1 ; c1ccc(-c2ccc(N(c3ccc4c(c3)oc3ccccc34)c3cccc(-c4ccccc4)c3-c3cc4ccccc4c4ccccc34)cc2)cc1 ; CC1(C)c2ccccc2-c2ccc(N(c3ccc(-c4ccccc4)cc3)c3cccc(-c4ccccc4)c3-c3cc4ccccc4c4ccccc34)cc21 ; CC1(C)c2ccccc2-c2ccc(N(c3cccc(-c4ccccc4)c3)c3cccc(-c4ccccc4)c3-c3cc4ccccc4c4ccccc34)cc21]

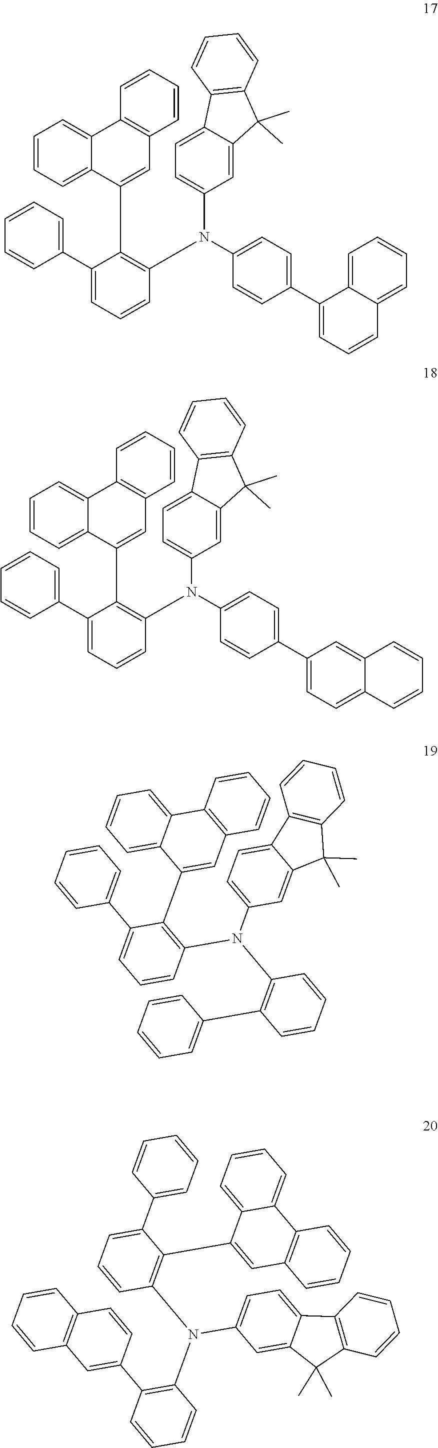 [SMILES: CC1(C)c2ccccc2-c2ccc(N(c3ccc(-c4cccc5ccccc45)cc3)c3cccc(-c4ccccc4)c3-c3cc4ccccc4c4ccccc34)cc21 ; CC1(C)c2ccccc2-c2ccc(N(c3ccc(-c4ccc5ccccc5c4)cc3)c3cccc(-c4ccccc4)c3-c3cc4ccccc4c4ccccc34)cc21 ; CC1(C)c2ccccc2-c2ccc(N(c3ccccc3-c3ccccc3)c3cccc(-c4ccccc4)c3-c3cc4ccccc4c4ccccc34)cc21 ; CC1(C)c2ccccc2-c2ccc(N(c3ccccc3-c3ccc4ccccc4c3)c3cccc(-c4ccccc4)c3-c3cc4ccccc4c4ccccc34)cc21]

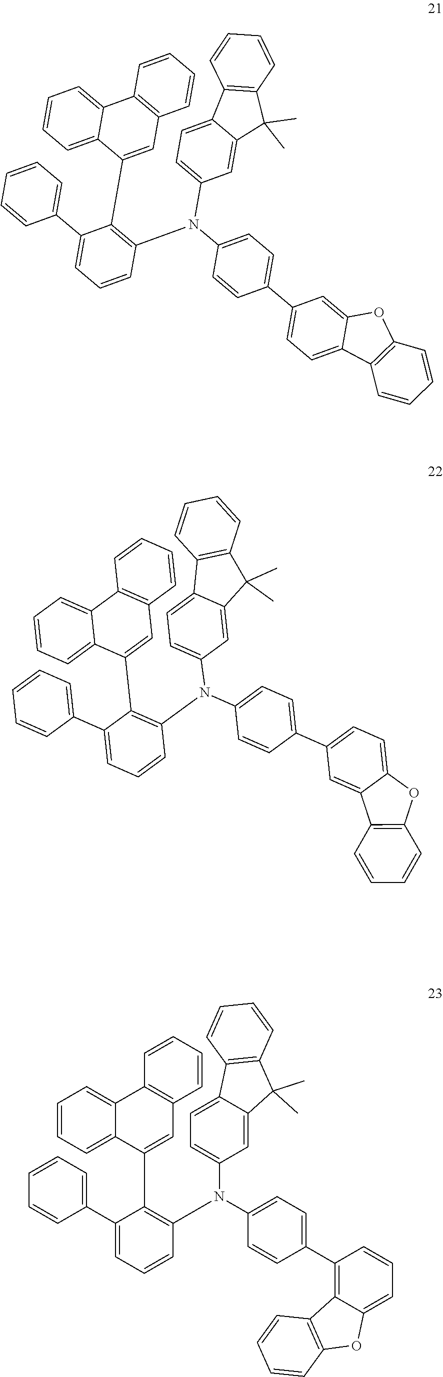 [SMILES: CC1(C)c2ccccc2-c2ccc(N(c3ccc(-c4ccc5c(c4)oc4ccccc45)cc3)c3cccc(-c4ccccc4)c3-c3cc4ccccc4c4ccccc34)cc21 ; CC1(C)c2ccccc2-c2ccc(N(c3ccc(-c4ccc5oc6ccccc6c5c4)cc3)c3cccc(-c4ccccc4)c3-c3cc4ccccc4c4ccccc34)cc21 ; CC1(C)c2ccccc2-c2ccc(N(c3ccc(-c4cccc5oc6ccccc6c45)cc3)c3cccc(-c4ccccc4)c3-c3cc4ccccc4c4ccccc34)cc21]

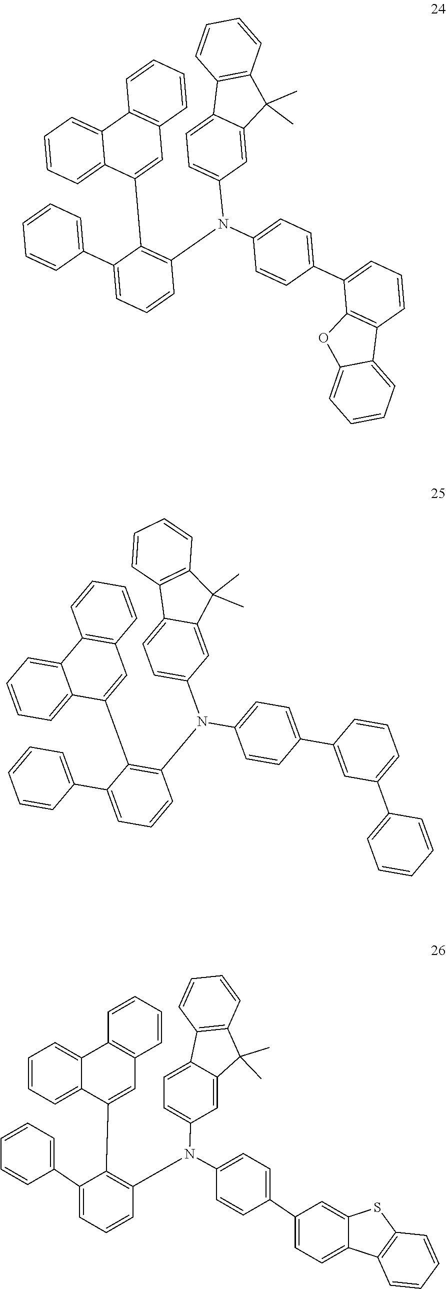 [SMILES: CC1(C)c2ccccc2-c2ccc(N(c3ccc(-c4cccc5c4oc4ccccc45)cc3)c3cccc(-c4ccccc4)c3-c3cc4ccccc4c4ccccc34)cc21 ; CC1(C)c2ccccc2-c2ccc(N(c3ccc(-c4cccc(-c5ccccc5)c4)cc3)c3cccc(-c4ccccc4)c3-c3cc4ccccc4c4ccccc34)cc21 ; CC1(C)c2ccccc2-c2ccc(N(c3ccc(-c4ccc5c(c4)sc4ccccc45)cc3)c3cccc(-c4ccccc4)c3-c3cc4ccccc4c4ccccc34)cc21]

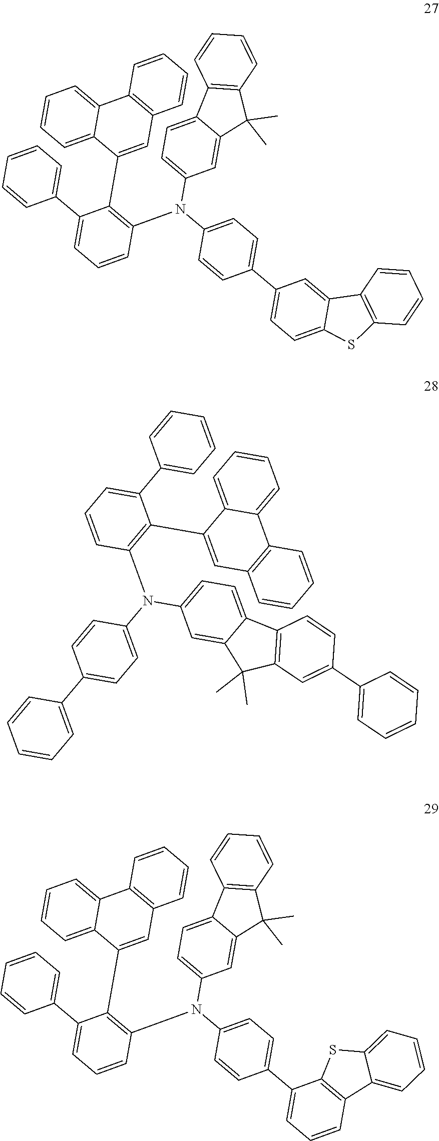 [SMILES: CC1(C)c2ccccc2-c2ccc(N(c3ccc(-c4ccc5sc6ccccc6c5c4)cc3)c3cccc(-c4ccccc4)c3-c3cc4ccccc4c4ccccc34)cc21 ; CC1(C)c2cc(-c3ccccc3)ccc2-c2ccc(N(c3ccc(-c4ccccc4)cc3)c3cccc(-c4ccccc4)c3-c3cc4ccccc4c4ccccc34)cc21 ; CC1(C)c2ccccc2-c2ccc(N(c3ccc(-c4cccc5c4sc4ccccc45)cc3)c3cccc(-c4ccccc4)c3-c3cc4ccccc4c4ccccc34)cc21]

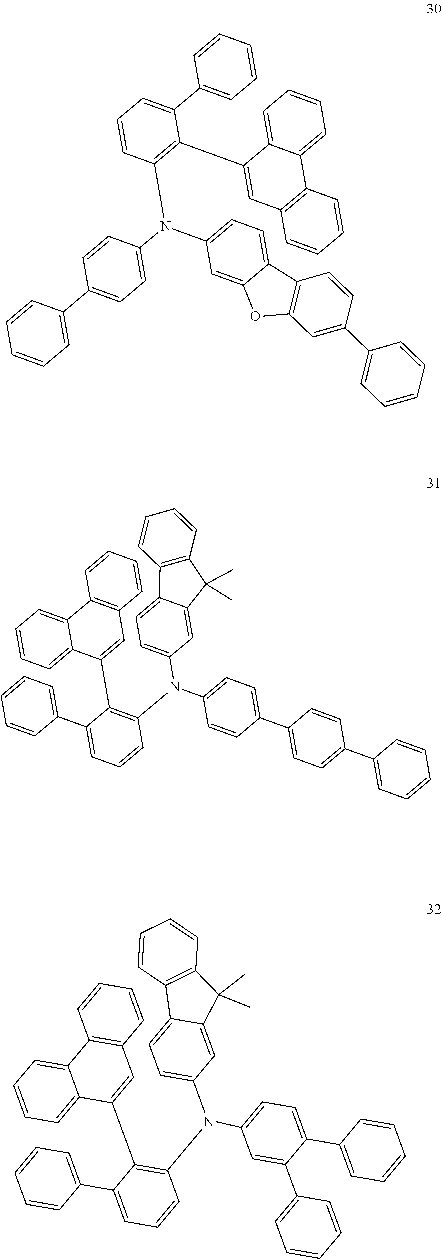 [SMILES: c1ccc(-c2ccc(N(c3ccc4c(c3)oc3cc(-c5ccccc5)ccc34)c3cccc(-c4ccccc4)c3-c3cc4ccccc4c4ccccc34)cc2)cc1 ; CC1(C)c2ccccc2-c2ccc(N(c3ccc(-c4ccc(-c5ccccc5)cc4)cc3)c3cccc(-c4ccccc4)c3-c3cc4ccccc4c4ccccc34)cc21 ; CC1(C)c2ccccc2-c2ccc(N(c3ccc(-c4ccccc4)c(-c4ccccc4)c3)c3cccc(-c4ccccc4)c3-c3cc4ccccc4c4ccccc34)cc21]

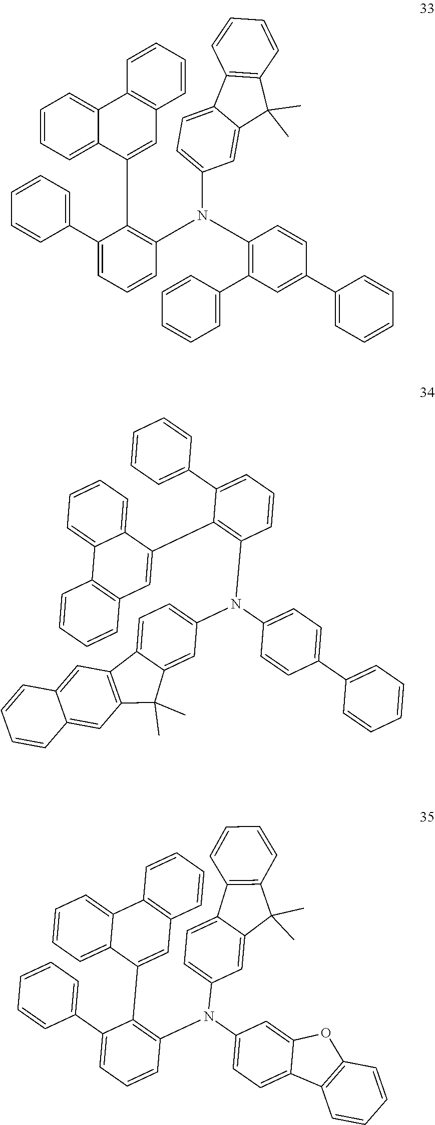 [SMILES: CC1(C)c2ccccc2-c2ccc(N(c3ccc(-c4ccccc4)cc3-c3ccccc3)c3cccc(-c4ccccc4)c3-c3cc4ccccc4c4ccccc34)cc21 ; CC1(C)c2cc(N(c3ccc(-c4ccccc4)cc3)c3cccc(-c4ccccc4)c3-c3cc4ccccc4c4ccccc34)ccc2-c2cc3ccccc3cc21 ; CC1(C)c2ccccc2-c2ccc(N(c3ccc4c(c3)oc3ccccc34)c3cccc(-c4ccccc4)c3-c3cc4ccccc4c4ccccc34)cc21]

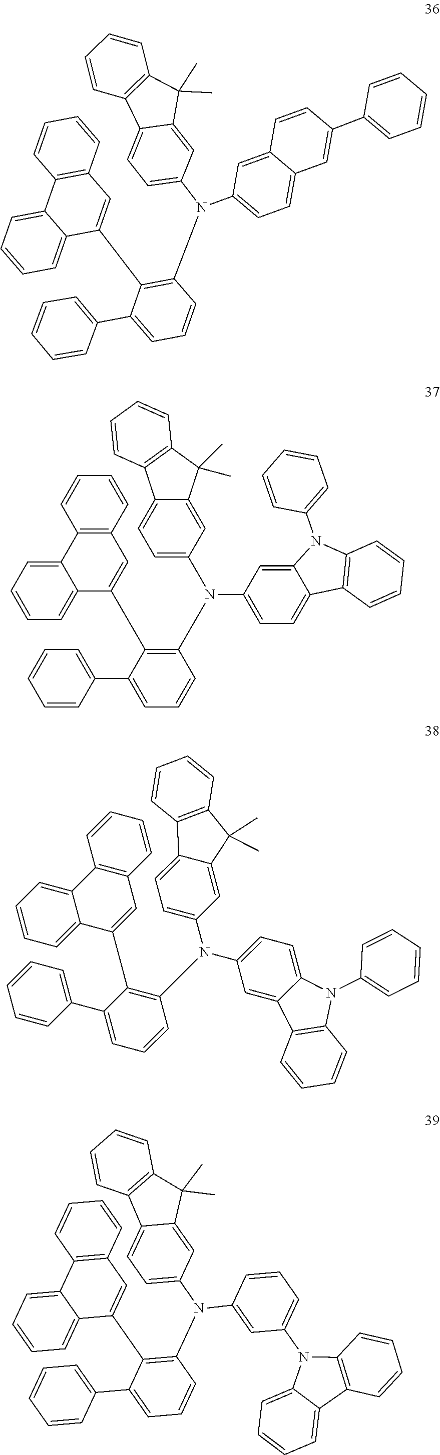 [SMILES: CC1(C)c2ccccc2-c2ccc(N(c3ccc4cc(-c5ccccc5)ccc4c3)c3cccc(-c4ccccc4)c3-c3cc4ccccc4c4ccccc34)cc21 ; CC1(C)c2ccccc2-c2ccc(N(c3ccc4c5ccccc5n(-c5ccccc5)c4c3)c3cccc(-c4ccccc4)c3-c3cc4ccccc4c4ccccc34)cc21 ; CC1(C)c2ccccc2-c2ccc(N(c3ccc4c(c3)c3ccccc3n4-c3ccccc3)c3cccc(-c4ccccc4)c3-c3cc4ccccc4c4ccccc34)cc21 ; CC1(C)c2ccccc2-c2ccc(N(c3cccc(-n4c5ccccc5c5ccccc54)c3)c3cccc(-c4ccccc4)c3-c3cc4ccccc4c4ccccc34)cc21]

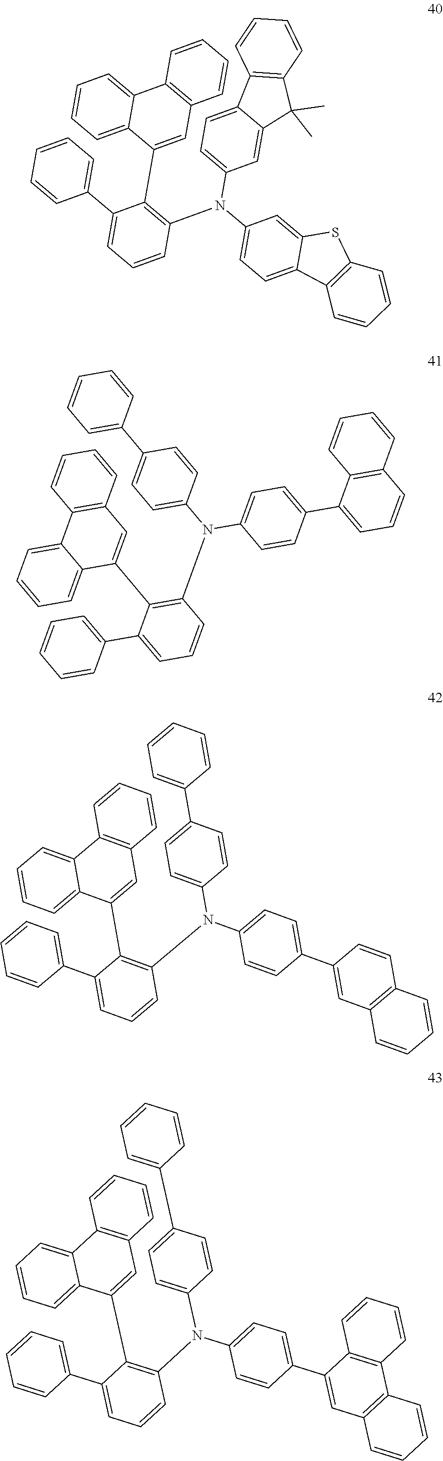 [SMILES: CC1(C)c2ccccc2-c2ccc(N(c3ccc4c(c3)sc3ccccc34)c3cccc(-c4ccccc4)c3-c3cc4ccccc4c4ccccc34)cc21 ; c1ccc(-c2ccc(N(c3ccc(-c4cccc5ccccc45)cc3)c3cccc(-c4ccccc4)c3-c3cc4ccccc4c4ccccc34)cc2)cc1 ; c1ccc(-c2ccc(N(c3ccc(-c4ccc5ccccc5c4)cc3)c3cccc(-c4ccccc4)c3-c3cc4ccccc4c4ccccc34)cc2)cc1 ; c1ccc(-c2ccc(N(c3ccc(-c4cc5ccccc5c5ccccc45)cc3)c3cccc(-c4ccccc4)c3-c3cc4ccccc4c4ccccc34)cc2)cc1]

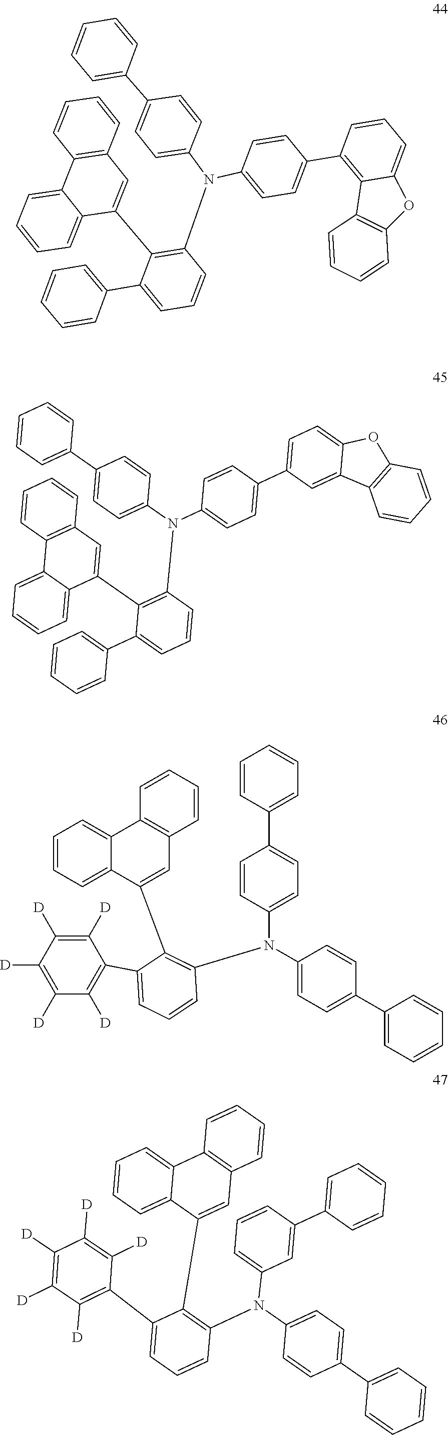 [SMILES: c1ccc(-c2ccc(N(c3ccc(-c4cccc5oc6ccccc6c45)cc3)c3cccc(-c4ccccc4)c3-c3cc4ccccc4c4ccccc34)cc2)cc1 ; c1ccc(-c2ccc(N(c3ccc(-c4ccc5oc6ccccc6c5c4)cc3)c3cccc(-c4ccccc4)c3-c3cc4ccccc4c4ccccc34)cc2)cc1 ; [2H]c1c([2H])c([2H])c(-c2cccc(N(c3ccc(-c4ccccc4)cc3)c3ccc(-c4ccccc4)cc3)c2-c2cc3ccccc3c3ccccc23)c([2H])c1[2H] ; [2H]c1c([2H])c([2H])c(-c2cccc(N(c3ccc(-c4ccccc4)cc3)c3cccc(-c4ccccc4)c3)c2-c2cc3ccccc3c3ccccc23)c([2H])c1[2H]]

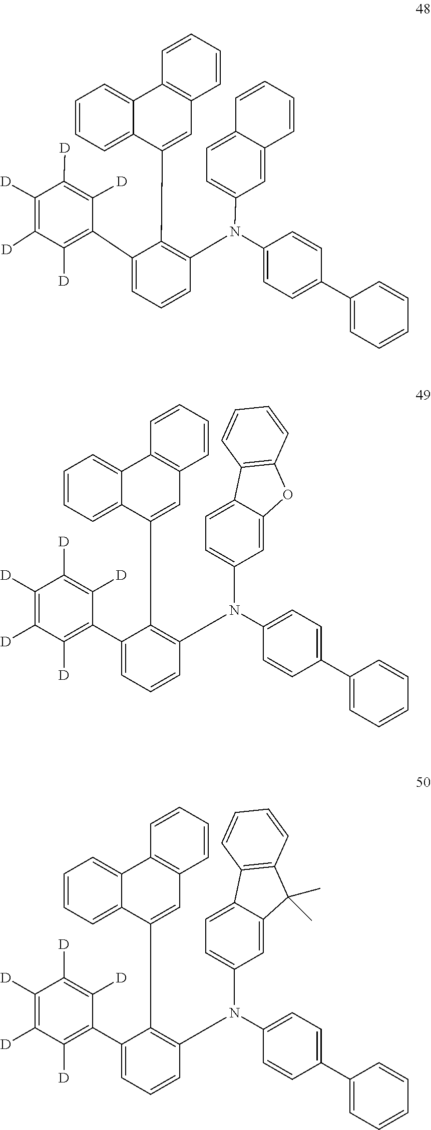 [SMILES: [2H]c1c([2H])c([2H])c(-c2cccc(N(c3ccc(-c4ccccc4)cc3)c3ccc4ccccc4c3)c2-c2cc3ccccc3c3ccccc23)c([2H])c1[2H] ; [2H]c1c([2H])c([2H])c(-c2cccc(N(c3ccc(-c4ccccc4)cc3)c3ccc4c(c3)oc3ccccc34)c2-c2cc3ccccc3c3ccccc23)c([2H])c1[2H] ; [2H]c1c([2H])c([2H])c(-c2cccc(N(c3ccc(-c4ccccc4)cc3)c3ccc4c(c3)C(C)(C)c3ccccc3-4)c2-c2cc3ccccc3c3ccccc23)c([2H])c1[2H]]

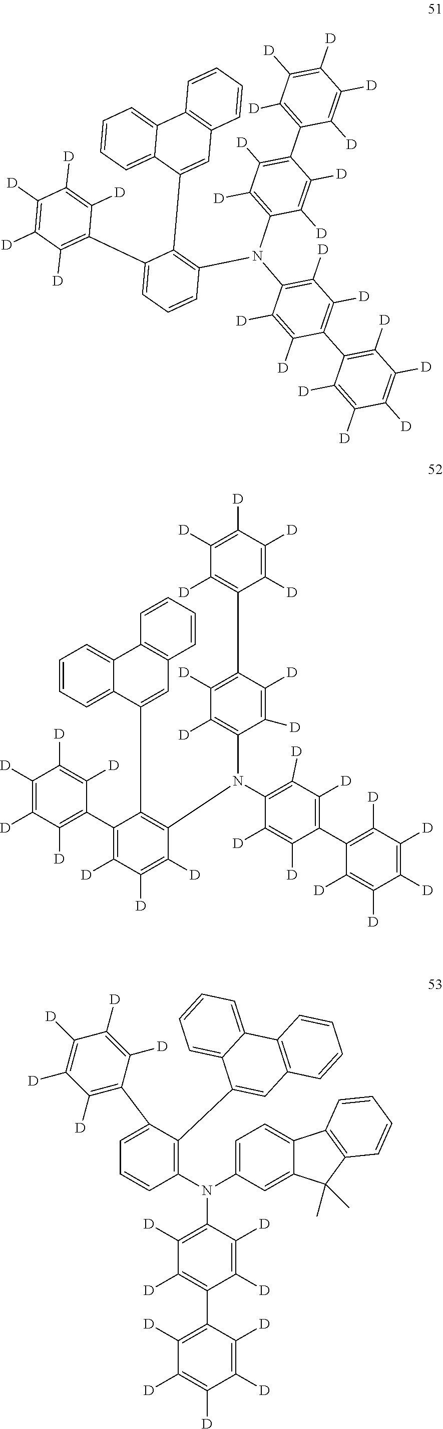

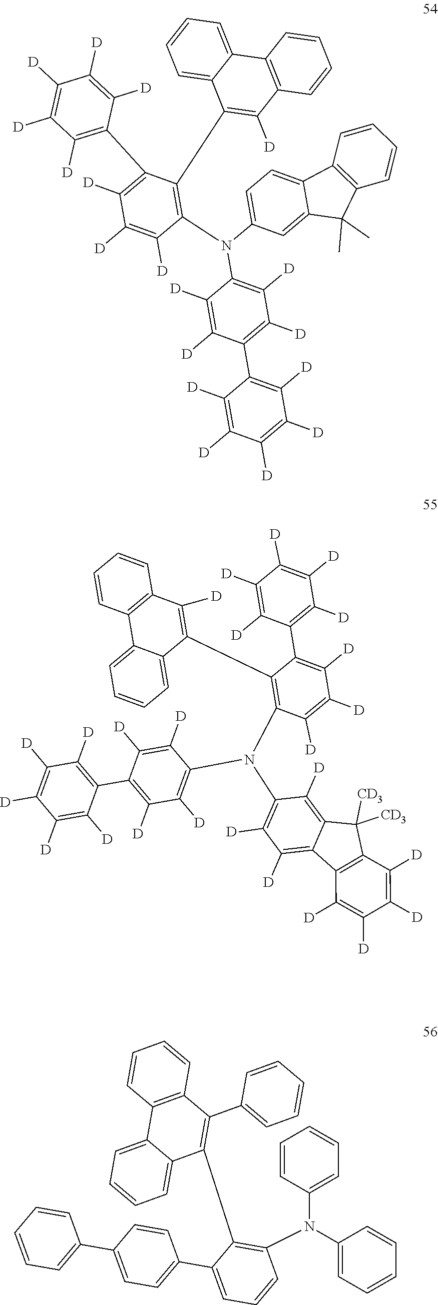

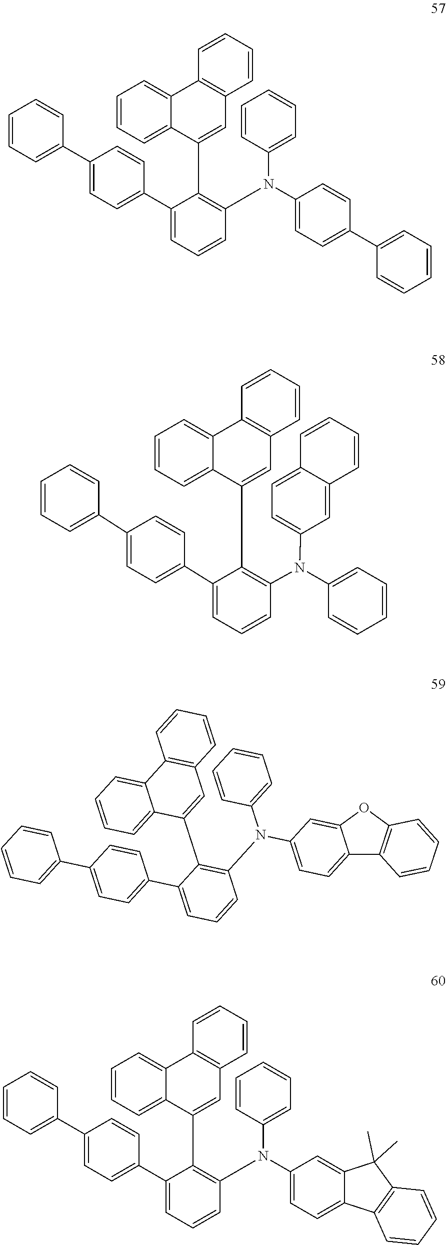 [SMILES: c1ccc(-c2ccc(-c3cccc(N(c4ccccc4)c4ccc(-c5ccccc5)cc4)c3-c3cc4ccccc4c4ccccc34)cc2)cc1 ; c1ccc(-c2ccc(-c3cccc(N(c4ccccc4)c4ccc5ccccc5c4)c3-c3cc4ccccc4c4ccccc34)cc2)cc1 ; c1ccc(-c2ccc(-c3cccc(N(c4ccccc4)c4ccc5c(c4)oc4ccccc45)c3-c3cc4ccccc4c4ccccc34)cc2)cc1 ; CC1(C)c2ccccc2-c2ccc(N(c3ccccc3)c3cccc(-c4ccc(-c5ccccc5)cc4)c3-c3cc4ccccc4c4ccccc34)cc21]

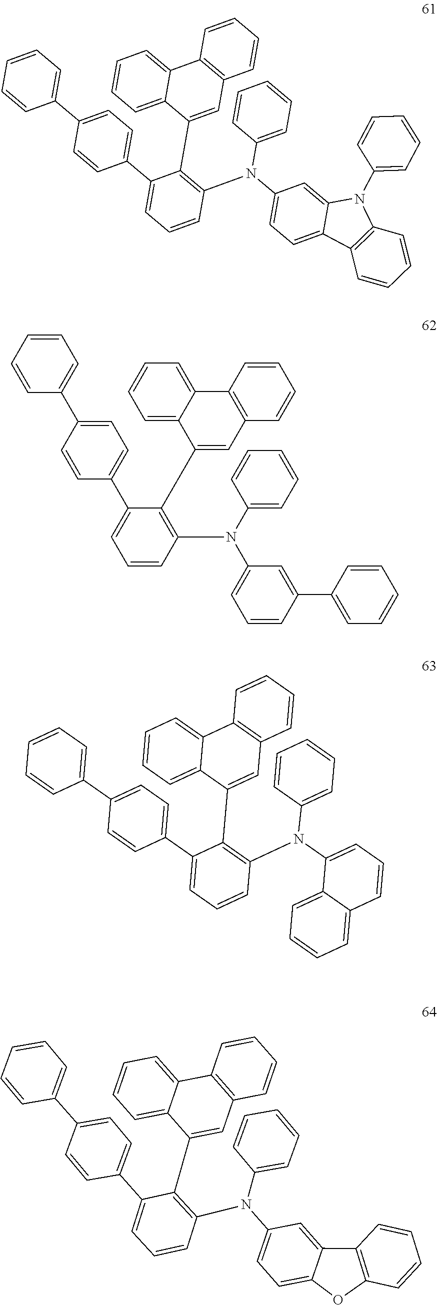 [SMILES: c1ccc(-c2ccc(-c3cccc(N(c4ccccc4)c4ccc5c6ccccc6n(-c6ccccc6)c5c4)c3-c3cc4ccccc4c4ccccc34)cc2)cc1 ; c1ccc(-c2ccc(-c3cccc(N(c4ccccc4)c4cccc(-c5ccccc5)c4)c3-c3cc4ccccc4c4ccccc34)cc2)cc1 ; c1ccc(-c2ccc(-c3cccc(N(c4ccccc4)c4cccc5ccccc45)c3-c3cc4ccccc4c4ccccc34)cc2)cc1 ; c1ccc(-c2ccc(-c3cccc(N(c4ccccc4)c4ccc5oc6ccccc6c5c4)c3-c3cc4ccccc4c4ccccc34)cc2)cc1]

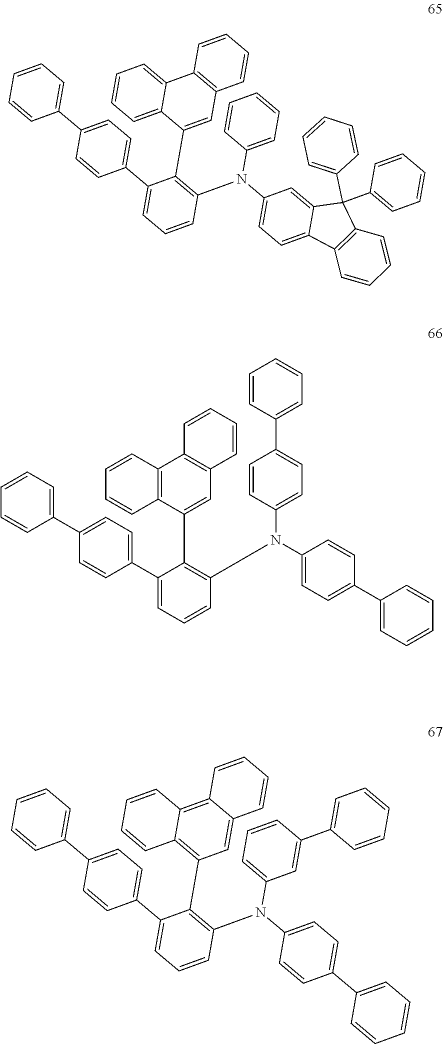 [SMILES: c1ccc(-c2ccc(-c3cccc(N(c4ccccc4)c4ccc5c(c4)C(c4ccccc4)(c4ccccc4)c4ccccc4-5)c3-c3cc4ccccc4c4ccccc34)cc2)cc1 ; c1ccc(-c2ccc(-c3cccc(N(c4ccc(-c5ccccc5)cc4)c4ccc(-c5ccccc5)cc4)c3-c3cc4ccccc4c4ccccc34)cc2)cc1 ; c1ccc(-c2ccc(-c3cccc(N(c4ccc(-c5ccccc5)cc4)c4cccc(-c5ccccc5)c4)c3-c3cc4ccccc4c4ccccc34)cc2)cc1]

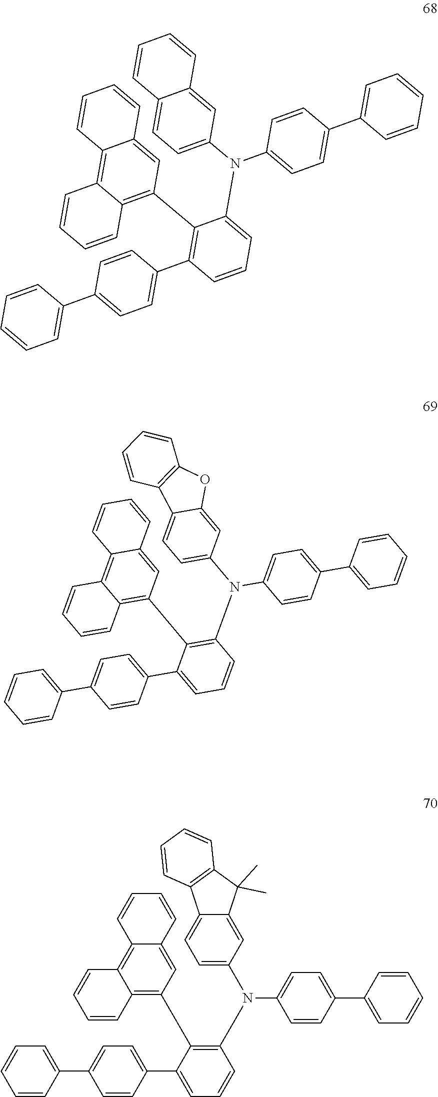 [SMILES: c1ccc(-c2ccc(-c3cccc(N(c4ccc(-c5ccccc5)cc4)c4ccc5ccccc5c4)c3-c3cc4ccccc4c4ccccc34)cc2)cc1 ; c1ccc(-c2ccc(-c3cccc(N(c4ccc(-c5ccccc5)cc4)c4ccc5c(c4)oc4ccccc45)c3-c3cc4ccccc4c4ccccc34)cc2)cc1 ; CC1(C)c2ccccc2-c2ccc(N(c3ccc(-c4ccccc4)cc3)c3cccc(-c4ccc(-c5ccccc5)cc4)c3-c3cc4ccccc4c4ccccc34)cc21]

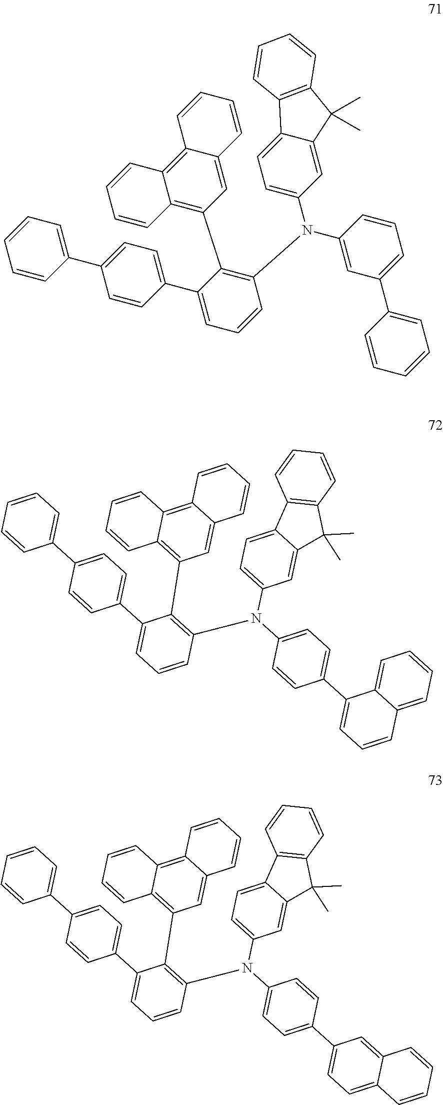 [SMILES: CC1(C)c2ccccc2-c2ccc(N(c3cccc(-c4ccccc4)c3)c3cccc(-c4ccc(-c5ccccc5)cc4)c3-c3cc4ccccc4c4ccccc34)cc21 ; CC1(C)c2ccccc2-c2ccc(N(c3ccc(-c4cccc5ccccc45)cc3)c3cccc(-c4ccc(-c5ccccc5)cc4)c3-c3cc4ccccc4c4ccccc34)cc21 ; CC1(C)c2ccccc2-c2ccc(N(c3ccc(-c4ccc5ccccc5c4)cc3)c3cccc(-c4ccc(-c5ccccc5)cc4)c3-c3cc4ccccc4c4ccccc34)cc21]

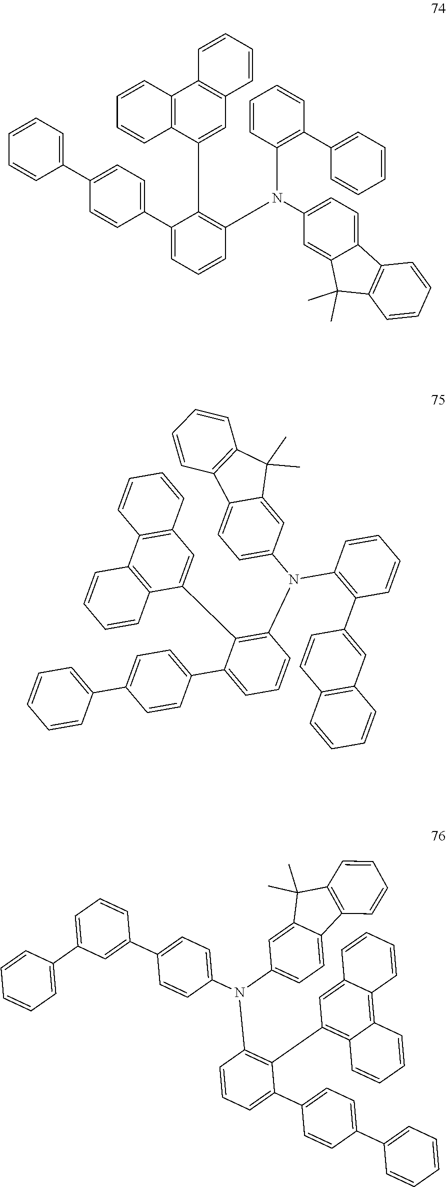 [SMILES: CC1(C)c2ccccc2-c2ccc(N(c3ccccc3-c3ccccc3)c3cccc(-c4ccc(-c5ccccc5)cc4)c3-c3cc4ccccc4c4ccccc34)cc21 ; CC1(C)c2ccccc2-c2ccc(N(c3ccccc3-c3ccc4ccccc4c3)c3cccc(-c4ccc(-c5ccccc5)cc4)c3-c3cc4ccccc4c4ccccc34)cc21 ; CC1(C)c2ccccc2-c2ccc(N(c3ccc(-c4cccc(-c5ccccc5)c4)cc3)c3cccc(-c4ccc(-c5ccccc5)cc4)c3-c3cc4ccccc4c4ccccc34)cc21]

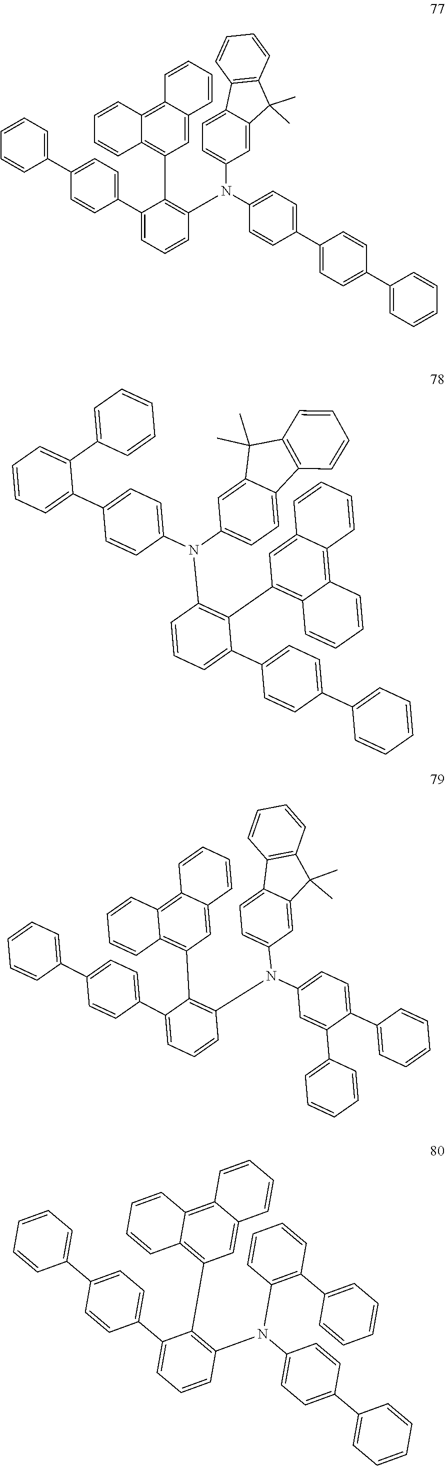 [SMILES: CC1(C)c2ccccc2-c2ccc(N(c3ccc(-c4ccc(-c5ccccc5)cc4)cc3)c3cccc(-c4ccc(-c5ccccc5)cc4)c3-c3cc4ccccc4c4ccccc34)cc21 ; CC1(C)c2ccccc2-c2ccc(N(c3ccc(-c4ccccc4-c4ccccc4)cc3)c3cccc(-c4ccc(-c5ccccc5)cc4)c3-c3cc4ccccc4c4ccccc34)cc21 ; CC1(C)c2ccccc2-c2ccc(N(c3ccc(-c4ccccc4)c(-c4ccccc4)c3)c3cccc(-c4ccc(-c5ccccc5)cc4)c3-c3cc4ccccc4c4ccccc34)cc21 ; c1ccc(-c2ccc(-c3cccc(N(c4ccc(-c5ccccc5)cc4)c4ccccc4-c4ccccc4)c3-c3cc4ccccc4c4ccccc34)cc2)cc1]

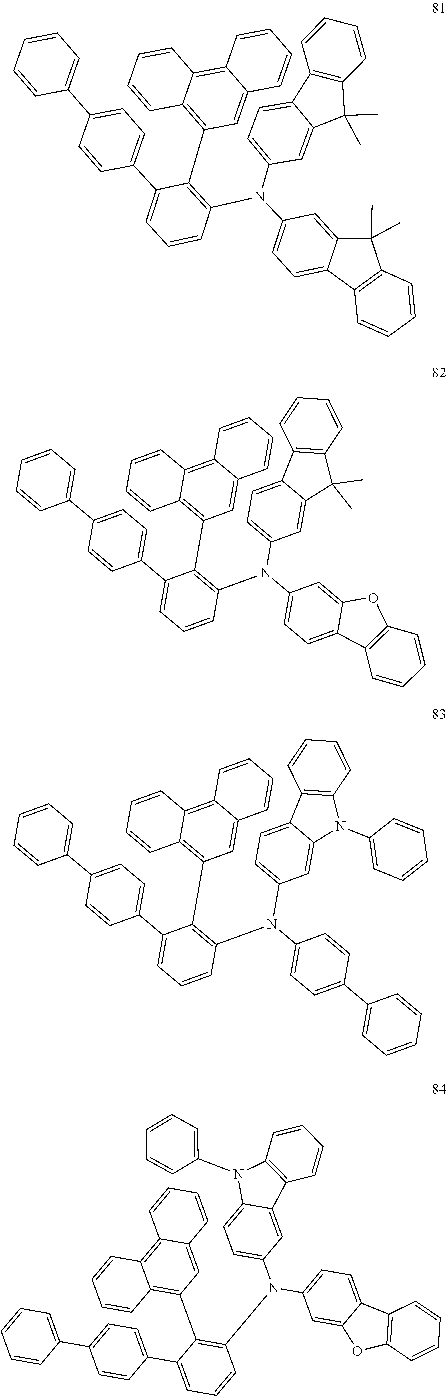 [SMILES: CC1(C)c2ccccc2-c2ccc(N(c3ccc4c(c3)C(C)(C)c3ccccc3-4)c3cccc(-c4ccc(-c5ccccc5)cc4)c3-c3cc4ccccc4c4ccccc34)cc21 ; CC1(C)c2ccccc2-c2ccc(N(c3ccc4c(c3)oc3ccccc34)c3cccc(-c4ccc(-c5ccccc5)cc4)c3-c3cc4ccccc4c4ccccc34)cc21 ; c1ccc(-c2ccc(-c3cccc(N(c4ccc(-c5ccccc5)cc4)c4ccc5c6ccccc6n(-c6ccccc6)c5c4)c3-c3cc4ccccc4c4ccccc34)cc2)cc1 ; c1ccc(-c2ccc(-c3cccc(N(c4ccc5c(c4)oc4ccccc45)c4ccc5c(c4)c4ccccc4n5-c4ccccc4)c3-c3cc4ccccc4c4ccccc34)cc2)cc1]

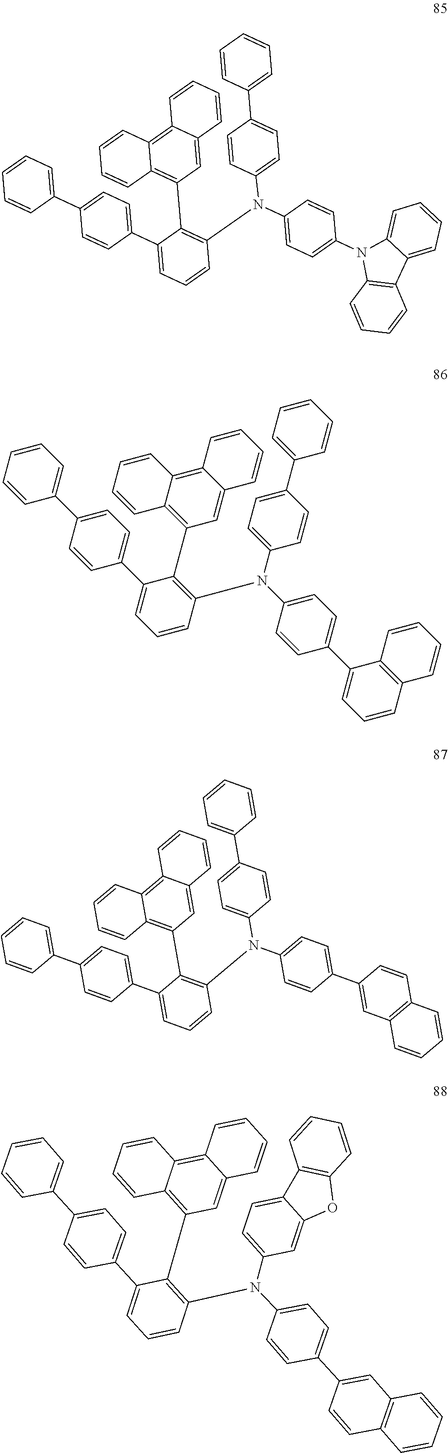 [SMILES: c1ccc(-c2ccc(-c3cccc(N(c4ccc(-c5ccccc5)cc4)c4ccc(-n5c6ccccc6c6ccccc65)cc4)c3-c3cc4ccccc4c4ccccc34)cc2)cc1 ; c1ccc(-c2ccc(-c3cccc(N(c4ccc(-c5ccccc5)cc4)c4ccc(-c5cccc6ccccc56)cc4)c3-c3cc4ccccc4c4ccccc34)cc2)cc1 ; c1ccc(-c2ccc(-c3cccc(N(c4ccc(-c5ccccc5)cc4)c4ccc(-c5ccc6ccccc6c5)cc4)c3-c3cc4ccccc4c4ccccc34)cc2)cc1 ; c1ccc(-c2ccc(-c3cccc(N(c4ccc(-c5ccc6ccccc6c5)cc4)c4ccc5c(c4)oc4ccccc45)c3-c3cc4ccccc4c4ccccc34)cc2)cc1]

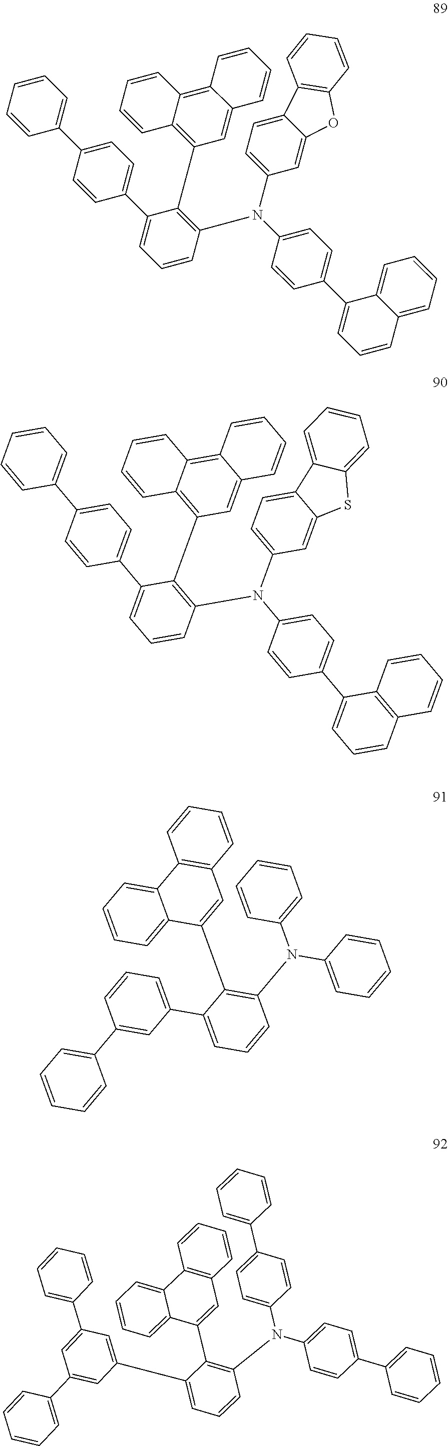 [SMILES: c1ccc(-c2ccc(-c3cccc(N(c4ccc(-c5cccc6ccccc56)cc4)c4ccc5c(c4)oc4ccccc45)c3-c3cc4ccccc4c4ccccc34)cc2)cc1 ; c1ccc(-c2ccc(-c3cccc(N(c4ccc(-c5cccc6ccccc56)cc4)c4ccc5c(c4)sc4ccccc45)c3-c3cc4ccccc4c4ccccc34)cc2)cc1 ; c1ccc(-c2cccc(-c3cccc(N(c4ccccc4)c4ccccc4)c3-c3cc4ccccc4c4ccccc34)c2)cc1 ; c1ccc(-c2ccc(N(c3ccc(-c4ccccc4)cc3)c3cccc(-c4cc(-c5ccccc5)cc(-c5ccccc5)c4)c3-c3cc4ccccc4c4ccccc34)cc2)cc1]

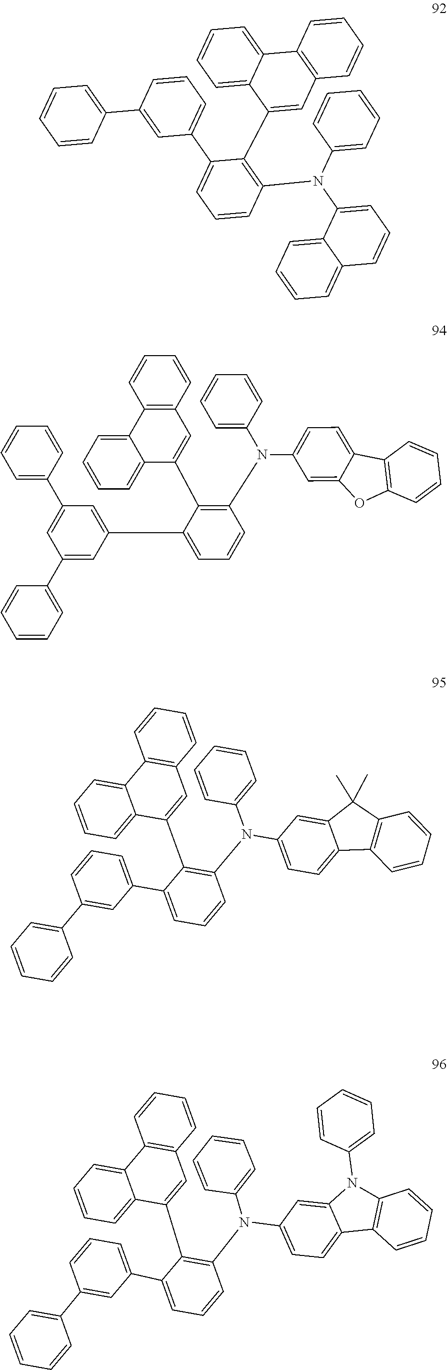 [SMILES: c1ccc(-c2cccc(-c3cccc(N(c4ccccc4)c4cccc5ccccc45)c3-c3cc4ccccc4c4ccccc34)c2)cc1 ; c1ccc(-c2cc(-c3ccccc3)cc(-c3cccc(N(c4ccccc4)c4ccc5c(c4)oc4ccccc45)c3-c3cc4ccccc4c4ccccc34)c2)cc1 ; CC1(C)c2ccccc2-c2ccc(N(c3ccccc3)c3cccc(-c4cccc(-c5ccccc5)c4)c3-c3cc4ccccc4c4ccccc34)cc21 ; c1ccc(-c2cccc(-c3cccc(N(c4ccccc4)c4ccc5c6ccccc6n(-c6ccccc6)c5c4)c3-c3cc4ccccc4c4ccccc34)c2)cc1]

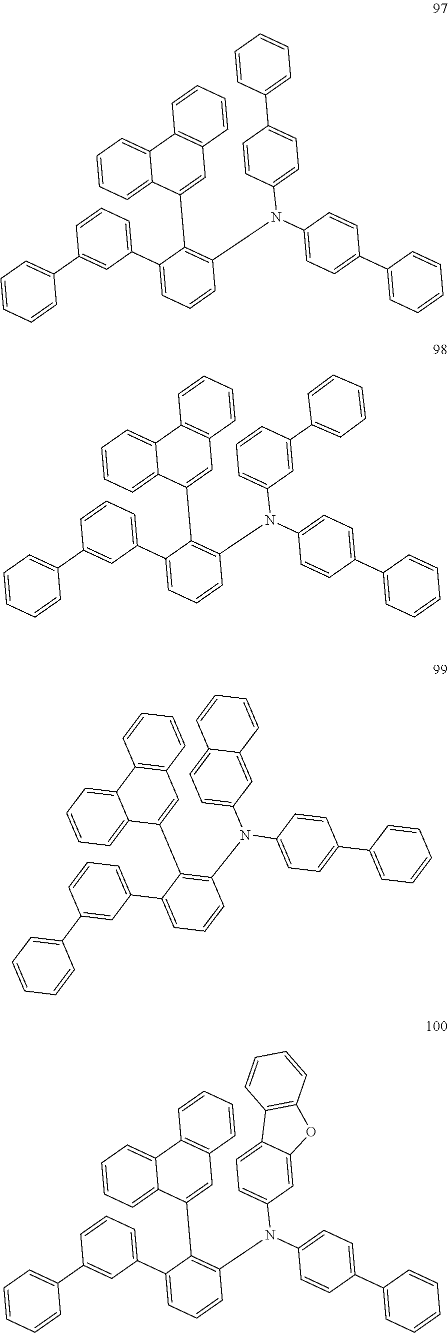 [SMILES: c1ccc(-c2ccc(N(c3ccc(-c4ccccc4)cc3)c3cccc(-c4cccc(-c5ccccc5)c4)c3-c3cc4ccccc4c4ccccc34)cc2)cc1 ; c1ccc(-c2ccc(N(c3cccc(-c4ccccc4)c3)c3cccc(-c4cccc(-c5ccccc5)c4)c3-c3cc4ccccc4c4ccccc34)cc2)cc1 ; c1ccc(-c2ccc(N(c3ccc4ccccc4c3)c3cccc(-c4cccc(-c5ccccc5)c4)c3-c3cc4ccccc4c4ccccc34)cc2)cc1 ; c1ccc(-c2ccc(N(c3ccc4c(c3)oc3ccccc34)c3cccc(-c4cccc(-c5ccccc5)c4)c3-c3cc4ccccc4c4ccccc34)cc2)cc1]

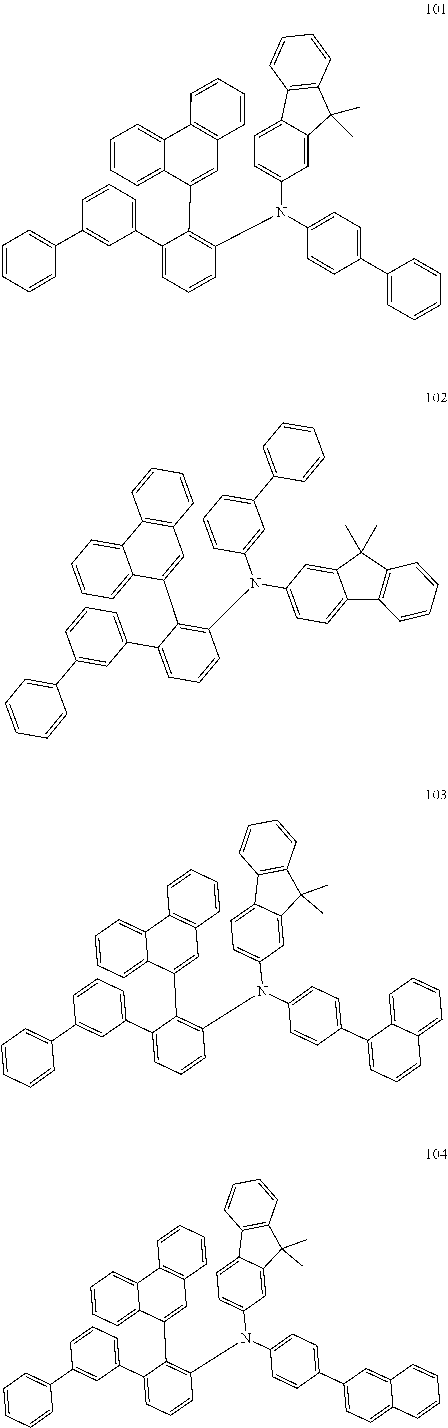 [SMILES: CC1(C)c2ccccc2-c2ccc(N(c3ccc(-c4ccccc4)cc3)c3cccc(-c4cccc(-c5ccccc5)c4)c3-c3cc4ccccc4c4ccccc34)cc21 ; CC1(C)c2ccccc2-c2ccc(N(c3cccc(-c4ccccc4)c3)c3cccc(-c4cccc(-c5ccccc5)c4)c3-c3cc4ccccc4c4ccccc34)cc21 ; CC1(C)c2ccccc2-c2ccc(N(c3ccc(-c4cccc5ccccc45)cc3)c3cccc(-c4cccc(-c5ccccc5)c4)c3-c3cc4ccccc4c4ccccc34)cc21 ; CC1(C)c2ccccc2-c2ccc(N(c3ccc(-c4ccc5ccccc5c4)cc3)c3cccc(-c4cccc(-c5ccccc5)c4)c3-c3cc4ccccc4c4ccccc34)cc21]

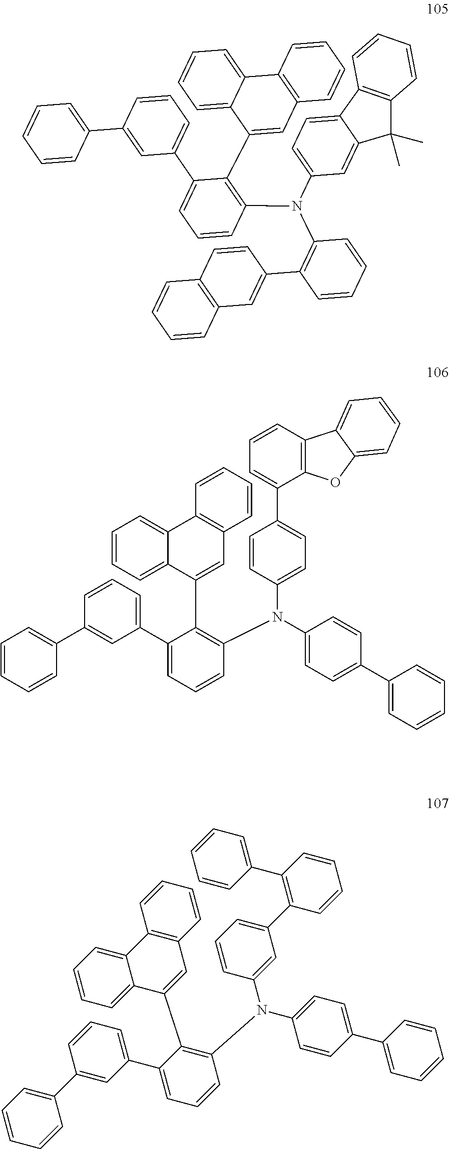 [SMILES: CC1(C)c2ccccc2-c2ccc(N(c3ccccc3-c3ccc4ccccc4c3)c3cccc(-c4cccc(-c5ccccc5)c4)c3-c3cc4ccccc4c4ccccc34)cc21 ; c1ccc(-c2ccc(N(c3ccc(-c4cccc5c4oc4ccccc45)cc3)c3cccc(-c4cccc(-c5ccccc5)c4)c3-c3cc4ccccc4c4ccccc34)cc2)cc1 ; c1ccc(-c2ccc(N(c3cccc(-c4ccccc4-c4ccccc4)c3)c3cccc(-c4cccc(-c5ccccc5)c4)c3-c3cc4ccccc4c4ccccc34)cc2)cc1]

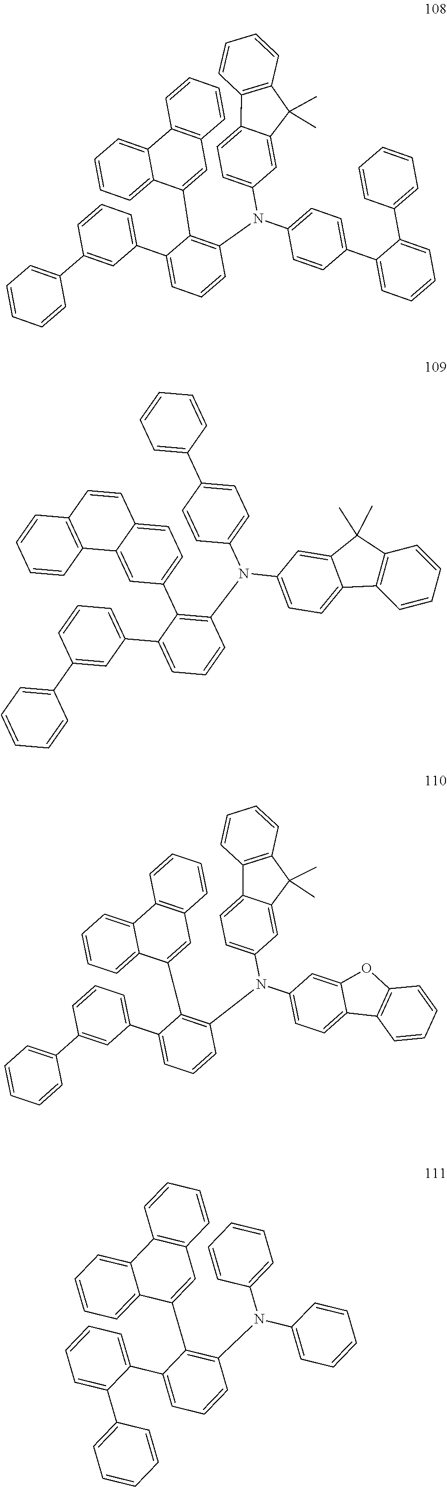 [SMILES: CC1(C)c2ccccc2-c2ccc(N(c3ccc(-c4ccccc4-c4ccccc4)cc3)c3cccc(-c4cccc(-c5ccccc5)c4)c3-c3cc4ccccc4c4ccccc34)cc21 ; CC1(C)c2ccccc2-c2ccc(N(c3ccc(-c4ccccc4)cc3)c3cccc(-c4cccc(-c5ccccc5)c4)c3-c3ccc4ccc5ccccc5c4c3)cc21 ; CC1(C)c2ccccc2-c2ccc(N(c3ccc4c(c3)oc3ccccc34)c3cccc(-c4cccc(-c5ccccc5)c4)c3-c3cc4ccccc4c4ccccc34)cc21 ; c1ccc(-c2ccccc2-c2cccc(N(c3ccccc3)c3ccccc3)c2-c2cc3ccccc3c3ccccc23)cc1]

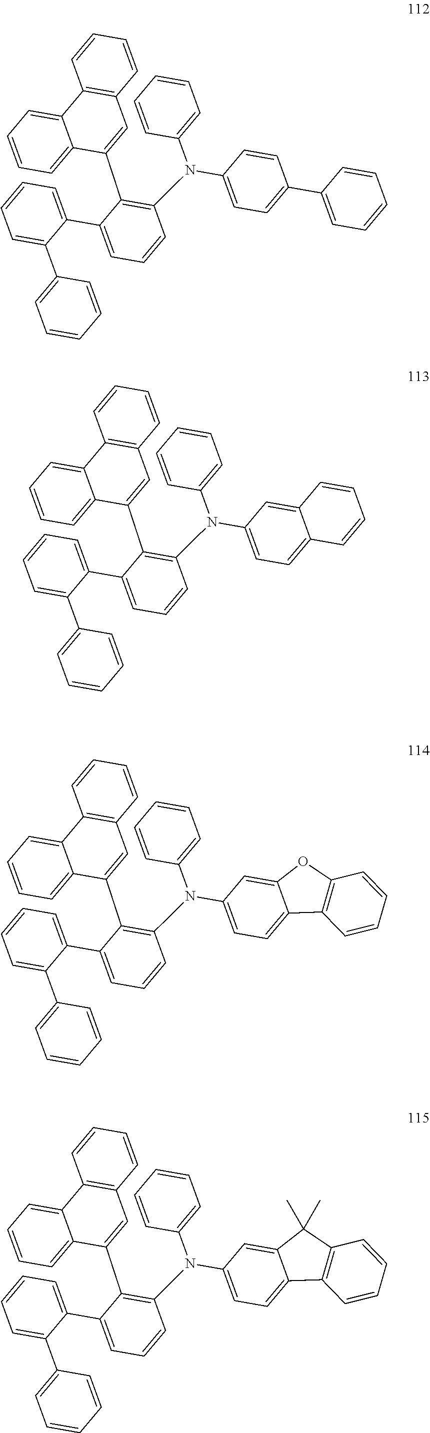 [SMILES: c1ccc(-c2ccc(N(c3ccccc3)c3cccc(-c4ccccc4-c4ccccc4)c3-c3cc4ccccc4c4ccccc34)cc2)cc1 ; c1ccc(-c2ccccc2-c2cccc(N(c3ccccc3)c3ccc4ccccc4c3)c2-c2cc3ccccc3c3ccccc23)cc1 ; c1ccc(-c2ccccc2-c2cccc(N(c3ccccc3)c3ccc4c(c3)oc3ccccc34)c2-c2cc3ccccc3c3ccccc23)cc1 ; CC1(C)c2ccccc2-c2ccc(N(c3ccccc3)c3cccc(-c4ccccc4-c4ccccc4)c3-c3cc4ccccc4c4ccccc34)cc21]

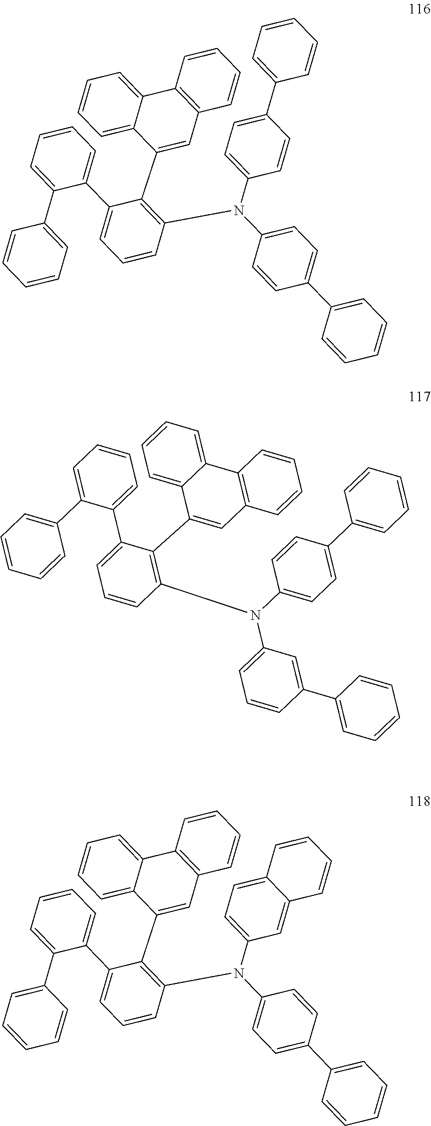 [SMILES: c1ccc(-c2ccc(N(c3ccc(-c4ccccc4)cc3)c3cccc(-c4ccccc4-c4ccccc4)c3-c3cc4ccccc4c4ccccc34)cc2)cc1 ; c1ccc(-c2ccc(N(c3cccc(-c4ccccc4)c3)c3cccc(-c4ccccc4-c4ccccc4)c3-c3cc4ccccc4c4ccccc34)cc2)cc1 ; c1ccc(-c2ccc(N(c3ccc4ccccc4c3)c3cccc(-c4ccccc4-c4ccccc4)c3-c3cc4ccccc4c4ccccc34)cc2)cc1]

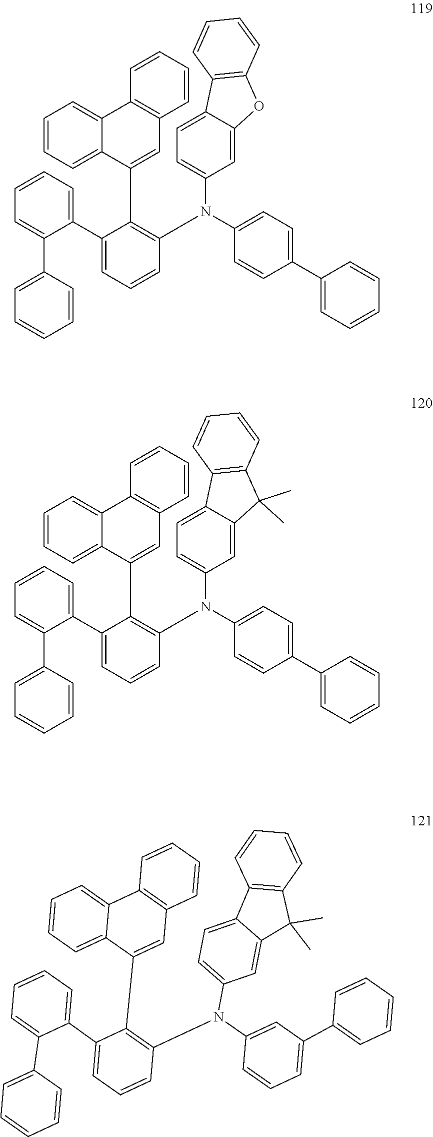 [SMILES: c1ccc(-c2ccc(N(c3ccc4c(c3)oc3ccccc34)c3cccc(-c4ccccc4-c4ccccc4)c3-c3cc4ccccc4c4ccccc34)cc2)cc1 ; CC1(C)c2ccccc2-c2ccc(N(c3ccc(-c4ccccc4)cc3)c3cccc(-c4ccccc4-c4ccccc4)c3-c3cc4ccccc4c4ccccc34)cc21 ; CC1(C)c2ccccc2-c2ccc(N(c3cccc(-c4ccccc4)c3)c3cccc(-c4ccccc4-c4ccccc4)c3-c3cc4ccccc4c4ccccc34)cc21]

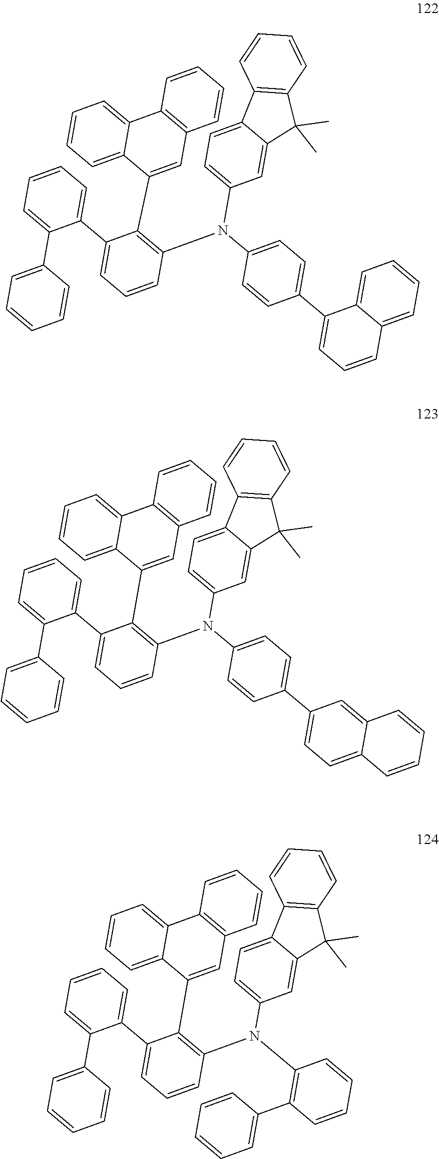 [SMILES: CC1(C)c2ccccc2-c2ccc(N(c3ccc(-c4cccc5ccccc45)cc3)c3cccc(-c4ccccc4-c4ccccc4)c3-c3cc4ccccc4c4ccccc34)cc21 ; CC1(C)c2ccccc2-c2ccc(N(c3ccc(-c4ccc5ccccc5c4)cc3)c3cccc(-c4ccccc4-c4ccccc4)c3-c3cc4ccccc4c4ccccc34)cc21 ; CC1(C)c2ccccc2-c2ccc(N(c3ccccc3-c3ccccc3)c3cccc(-c4ccccc4-c4ccccc4)c3-c3cc4ccccc4c4ccccc34)cc21]

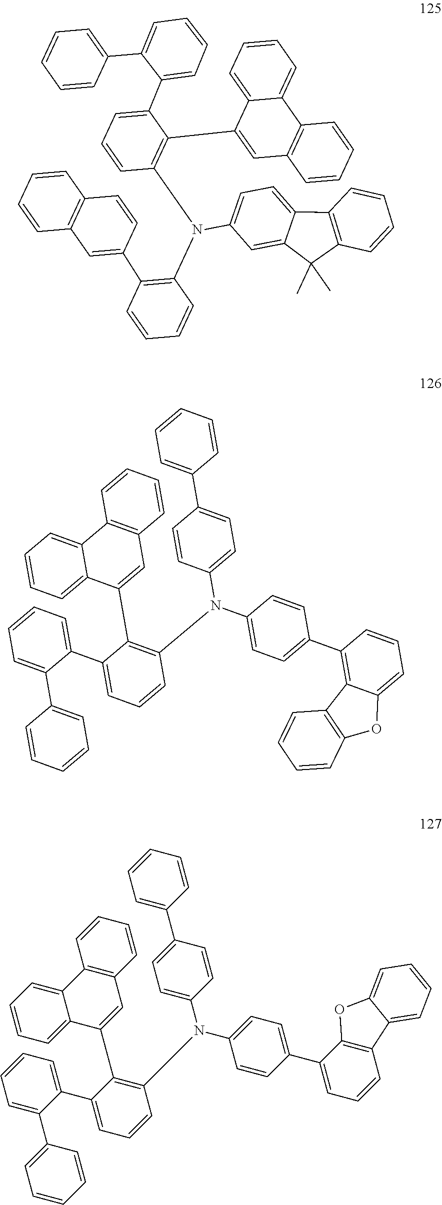 [SMILES: CC1(C)c2ccccc2-c2ccc(N(c3ccccc3-c3ccc4ccccc4c3)c3cccc(-c4ccccc4-c4ccccc4)c3-c3cc4ccccc4c4ccccc34)cc21 ; c1ccc(-c2ccc(N(c3ccc(-c4cccc5oc6ccccc6c45)cc3)c3cccc(-c4ccccc4-c4ccccc4)c3-c3cc4ccccc4c4ccccc34)cc2)cc1 ; c1ccc(-c2ccc(N(c3ccc(-c4cccc5c4oc4ccccc45)cc3)c3cccc(-c4ccccc4-c4ccccc4)c3-c3cc4ccccc4c4ccccc34)cc2)cc1]

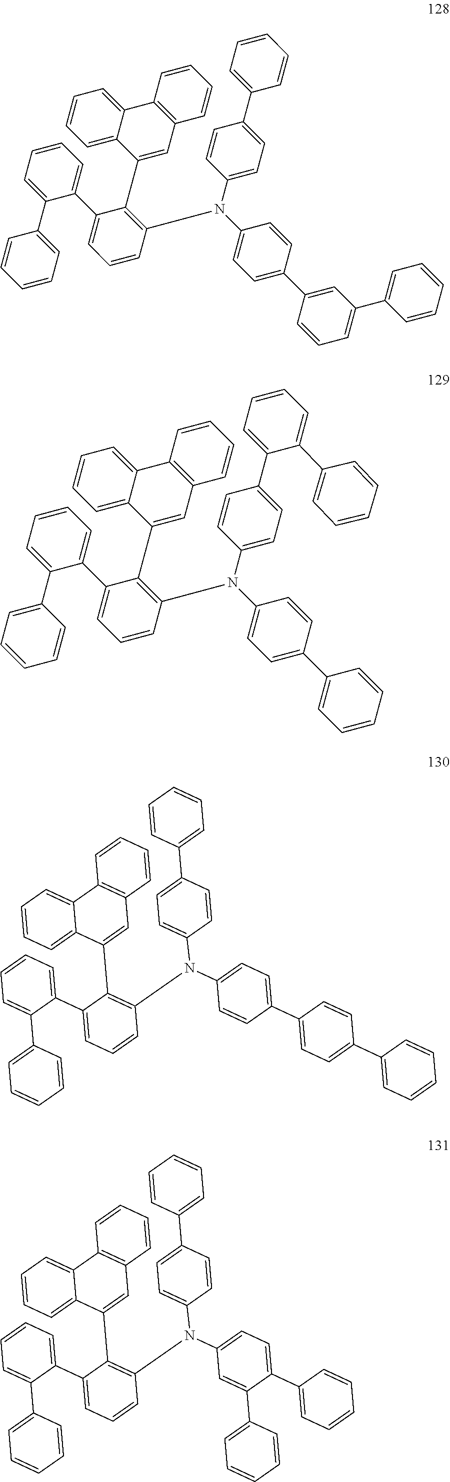 [SMILES: c1ccc(-c2ccc(N(c3ccc(-c4cccc(-c5ccccc5)c4)cc3)c3cccc(-c4ccccc4-c4ccccc4)c3-c3cc4ccccc4c4ccccc34)cc2)cc1 ; c1ccc(-c2ccc(N(c3ccc(-c4ccccc4-c4ccccc4)cc3)c3cccc(-c4ccccc4-c4ccccc4)c3-c3cc4ccccc4c4ccccc34)cc2)cc1 ; c1ccc(-c2ccc(-c3ccc(N(c4ccc(-c5ccccc5)cc4)c4cccc(-c5ccccc5-c5ccccc5)c4-c4cc5ccccc5c5ccccc45)cc3)cc2)cc1 ; c1ccc(-c2ccc(N(c3ccc(-c4ccccc4)c(-c4ccccc4)c3)c3cccc(-c4ccccc4-c4ccccc4)c3-c3cc4ccccc4c4ccccc34)cc2)cc1]

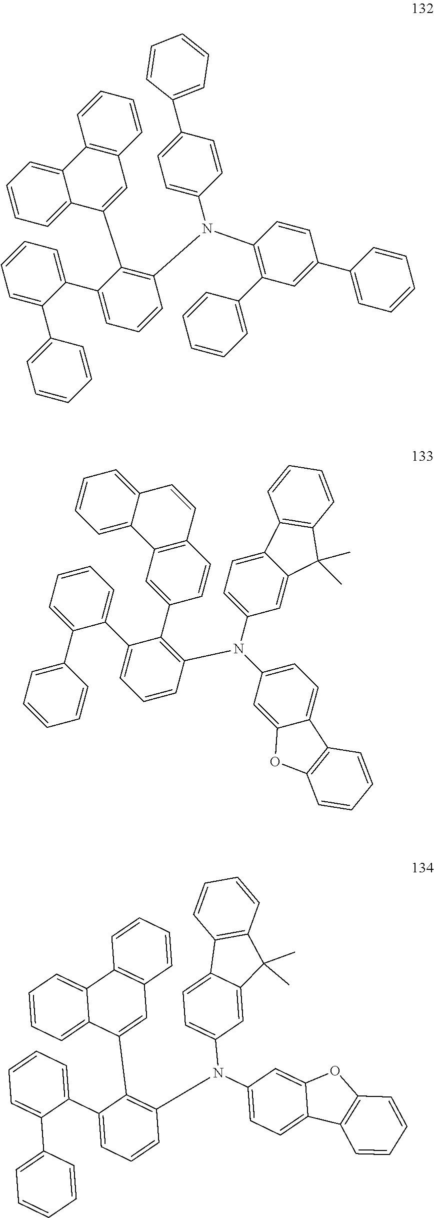 [SMILES: c1ccc(-c2ccc(N(c3ccc(-c4ccccc4)cc3-c3ccccc3)c3cccc(-c4ccccc4-c4ccccc4)c3-c3cc4ccccc4c4ccccc34)cc2)cc1 ; CC1(C)c2ccccc2-c2ccc(N(c3ccc4c(c3)oc3ccccc34)c3cccc(-c4ccccc4-c4ccccc4)c3-c3ccc4ccc5ccccc5c4c3)cc21 ; CC1(C)c2ccccc2-c2ccc(N(c3ccc4c(c3)oc3ccccc34)c3cccc(-c4ccccc4-c4ccccc4)c3-c3cc4ccccc4c4ccccc34)cc21]

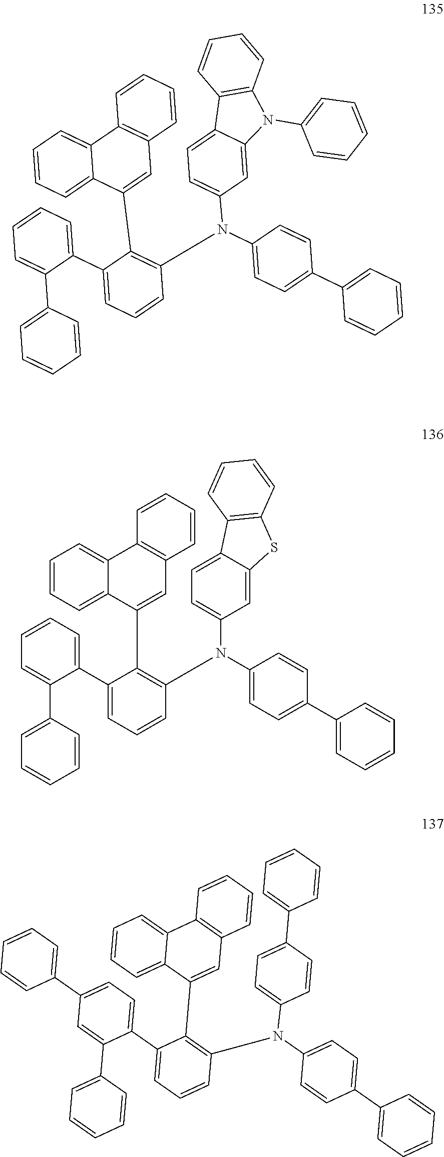 [SMILES: c1ccc(-c2ccc(N(c3ccc4c5ccccc5n(-c5ccccc5)c4c3)c3cccc(-c4ccccc4-c4ccccc4)c3-c3cc4ccccc4c4ccccc34)cc2)cc1 ; c1ccc(-c2ccc(N(c3ccc4c(c3)sc3ccccc34)c3cccc(-c4ccccc4-c4ccccc4)c3-c3cc4ccccc4c4ccccc34)cc2)cc1 ; c1ccc(-c2ccc(N(c3ccc(-c4ccccc4)cc3)c3cccc(-c4ccc(-c5ccccc5)cc4-c4ccccc4)c3-c3cc4ccccc4c4ccccc34)cc2)cc1]

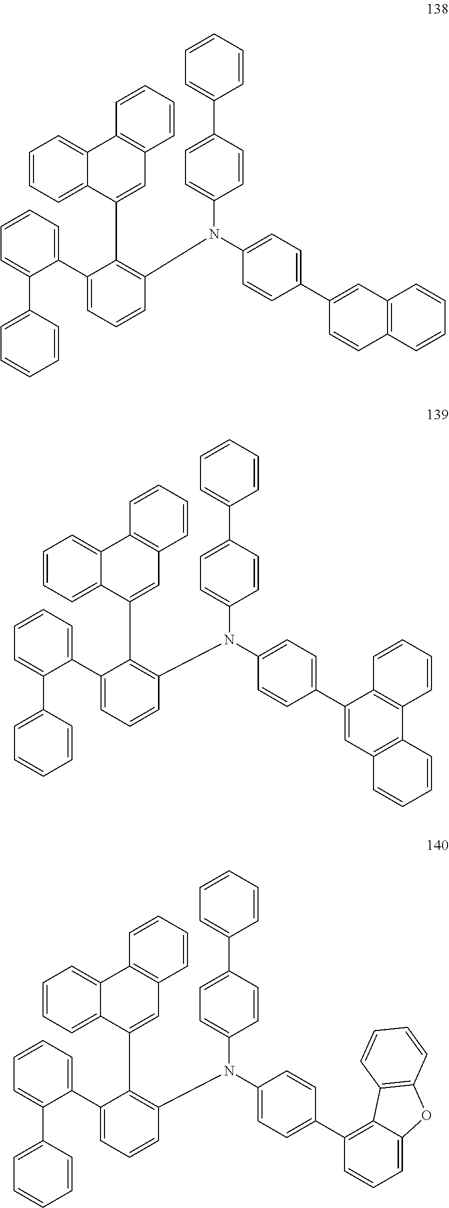 [SMILES: c1ccc(-c2ccc(N(c3ccc(-c4ccc5ccccc5c4)cc3)c3cccc(-c4ccccc4-c4ccccc4)c3-c3cc4ccccc4c4ccccc34)cc2)cc1 ; c1ccc(-c2ccc(N(c3ccc(-c4cc5ccccc5c5ccccc45)cc3)c3cccc(-c4ccccc4-c4ccccc4)c3-c3cc4ccccc4c4ccccc34)cc2)cc1 ; c1ccc(-c2ccc(N(c3ccc(-c4cccc5oc6ccccc6c45)cc3)c3cccc(-c4ccccc4-c4ccccc4)c3-c3cc4ccccc4c4ccccc34)cc2)cc1]

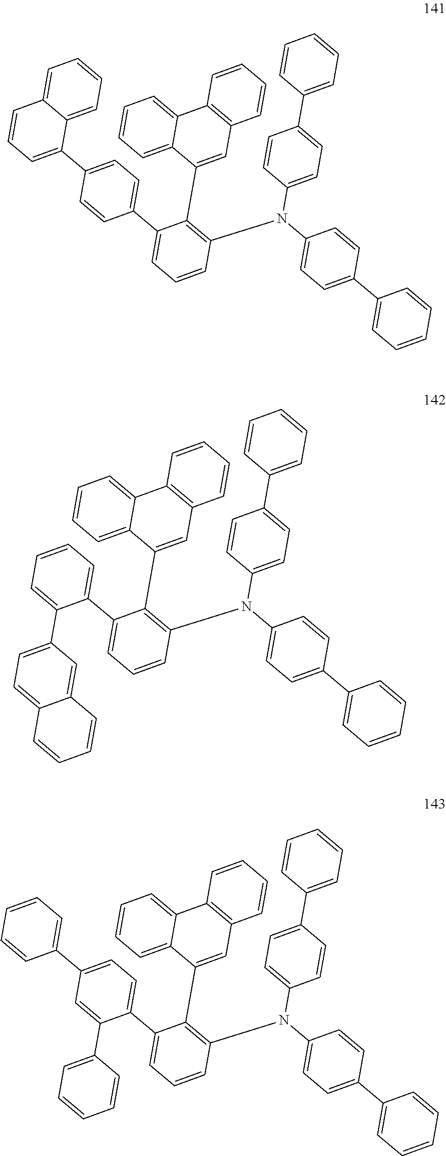 [SMILES: c1ccc(-c2ccc(N(c3ccc(-c4ccccc4)cc3)c3cccc(-c4ccc(-c5cccc6ccccc56)cc4)c3-c3cc4ccccc4c4ccccc34)cc2)cc1 ; c1ccc(-c2ccc(N(c3ccc(-c4ccccc4)cc3)c3cccc(-c4ccccc4-c4ccc5ccccc5c4)c3-c3cc4ccccc4c4ccccc34)cc2)cc1 ; c1ccc(-c2ccc(N(c3ccc(-c4ccccc4)cc3)c3cccc(-c4ccc(-c5ccccc5)cc4-c4ccccc4)c3-c3cc4ccccc4c4ccccc34)cc2)cc1]

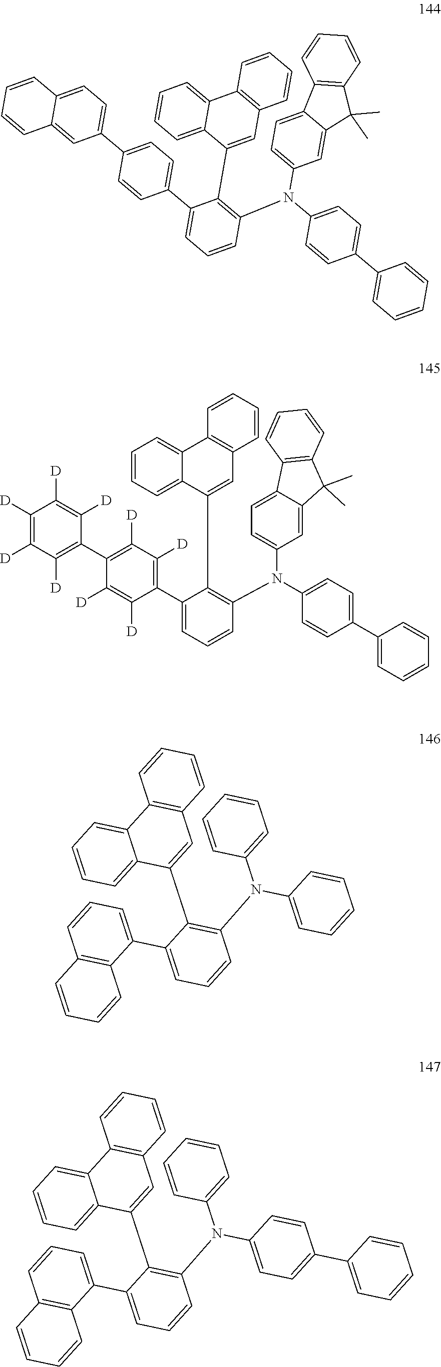 [SMILES: CC1(C)c2ccccc2-c2ccc(N(c3ccc(-c4ccccc4)cc3)c3cccc(-c4ccc(-c5ccc6ccccc6c5)cc4)c3-c3cc4ccccc4c4ccccc34)cc21 ; [2H]c1c([2H])c([2H])c(-c2c([2H])c([2H])c(-c3cccc(N(c4ccc(-c5ccccc5)cc4)c4ccc5c(c4)C(C)(C)c4ccccc4-5)c3-c3cc4ccccc4c4ccccc34)c([2H])c2[2H])c([2H])c1[2H] ; c1ccc(N(c2ccccc2)c2cccc(-c3cccc4ccccc34)c2-c2cc3ccccc3c3ccccc23)cc1 ; c1ccc(-c2ccc(N(c3ccccc3)c3cccc(-c4cccc5ccccc45)c3-c3cc4ccccc4c4ccccc34)cc2)cc1]

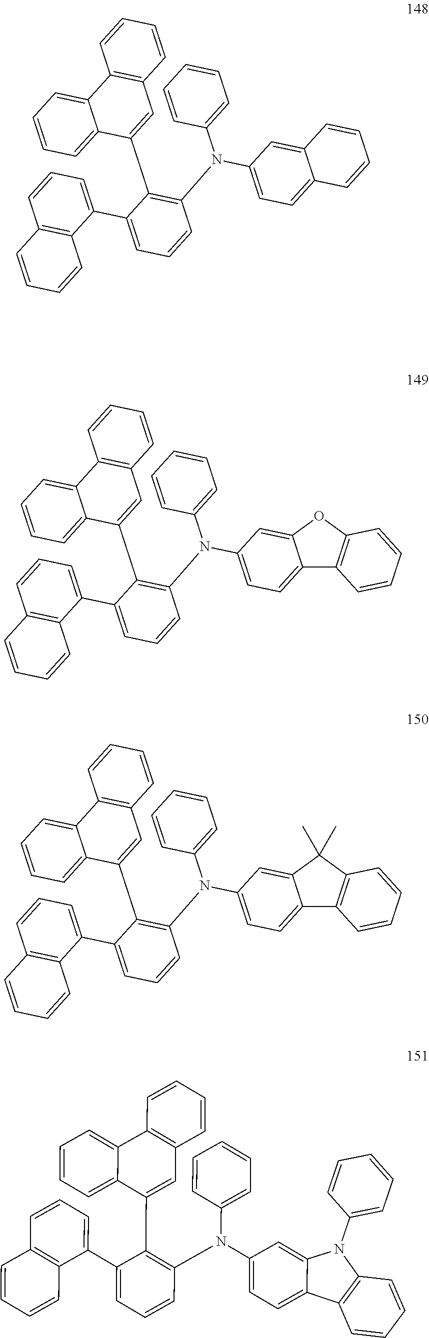 [SMILES: c1ccc(N(c2ccc3ccccc3c2)c2cccc(-c3cccc4ccccc34)c2-c2cc3ccccc3c3ccccc23)cc1 ; c1ccc(N(c2ccc3c(c2)oc2ccccc23)c2cccc(-c3cccc4ccccc34)c2-c2cc3ccccc3c3ccccc23)cc1 ; CC1(C)c2ccccc2-c2ccc(N(c3ccccc3)c3cccc(-c4cccc5ccccc45)c3-c3cc4ccccc4c4ccccc34)cc21 ; c1ccc(N(c2ccc3c4ccccc4n(-c4ccccc4)c3c2)c2cccc(-c3cccc4ccccc34)c2-c2cc3ccccc3c3ccccc23)cc1]

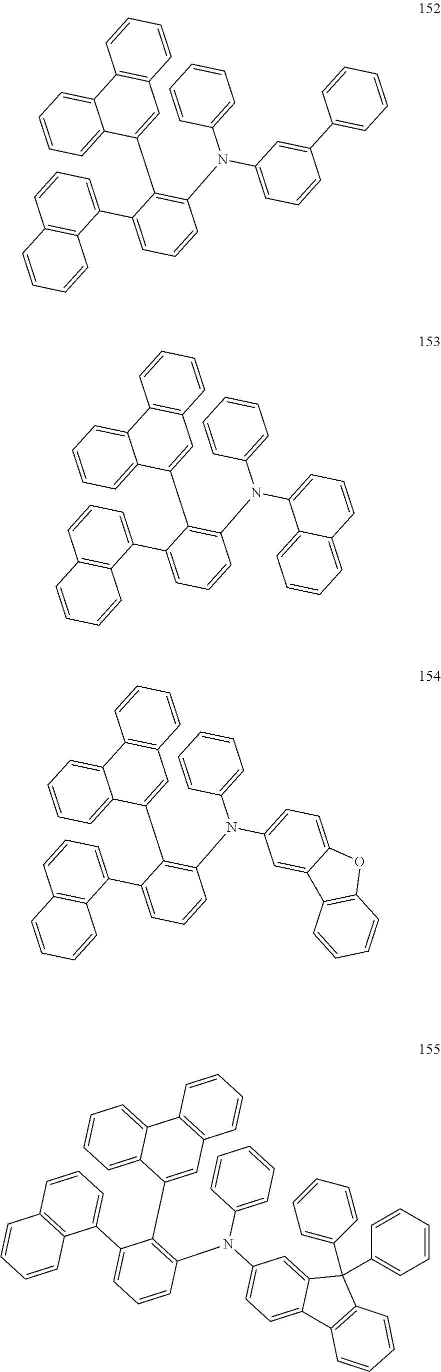 [SMILES: c1ccc(-c2cccc(N(c3ccccc3)c3cccc(-c4cccc5ccccc45)c3-c3cc4ccccc4c4ccccc34)c2)cc1 ; c1ccc(N(c2cccc(-c3cccc4ccccc34)c2-c2cc3ccccc3c3ccccc23)c2cccc3ccccc23)cc1 ; c1ccc(N(c2ccc3oc4ccccc4c3c2)c2cccc(-c3cccc4ccccc34)c2-c2cc3ccccc3c3ccccc23)cc1 ; c1ccc(N(c2ccc3c(c2)C(c2ccccc2)(c2ccccc2)c2ccccc2-3)c2cccc(-c3cccc4ccccc34)c2-c2cc3ccccc3c3ccccc23)cc1]

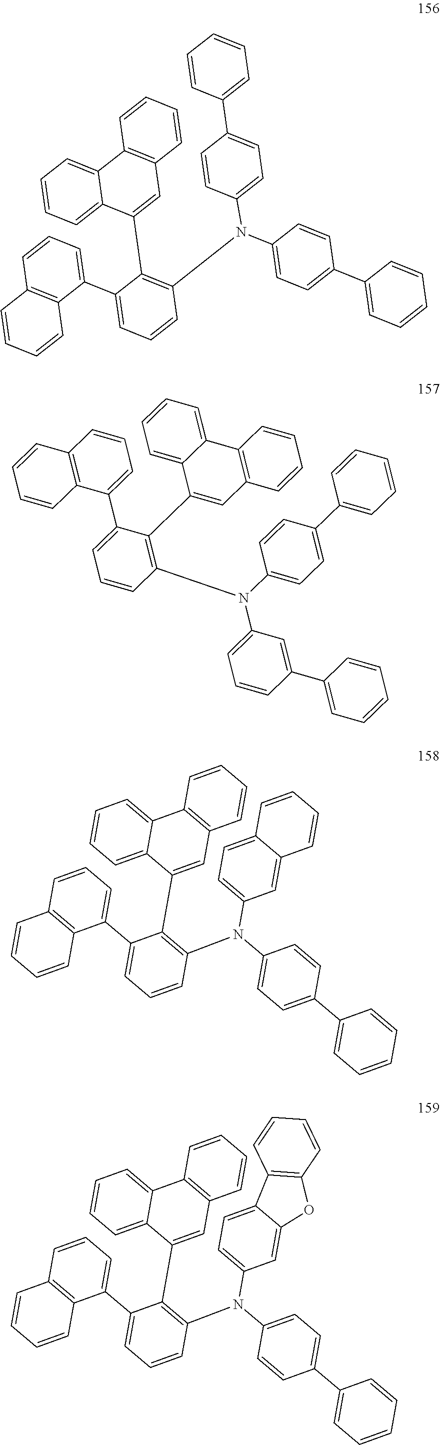 [SMILES: c1ccc(-c2ccc(N(c3ccc(-c4ccccc4)cc3)c3cccc(-c4cccc5ccccc45)c3-c3cc4ccccc4c4ccccc34)cc2)cc1 ; c1ccc(-c2ccc(N(c3cccc(-c4ccccc4)c3)c3cccc(-c4cccc5ccccc45)c3-c3cc4ccccc4c4ccccc34)cc2)cc1 ; c1ccc(-c2ccc(N(c3ccc4ccccc4c3)c3cccc(-c4cccc5ccccc45)c3-c3cc4ccccc4c4ccccc34)cc2)cc1 ; c1ccc(-c2ccc(N(c3ccc4c(c3)oc3ccccc34)c3cccc(-c4cccc5ccccc45)c3-c3cc4ccccc4c4ccccc34)cc2)cc1]

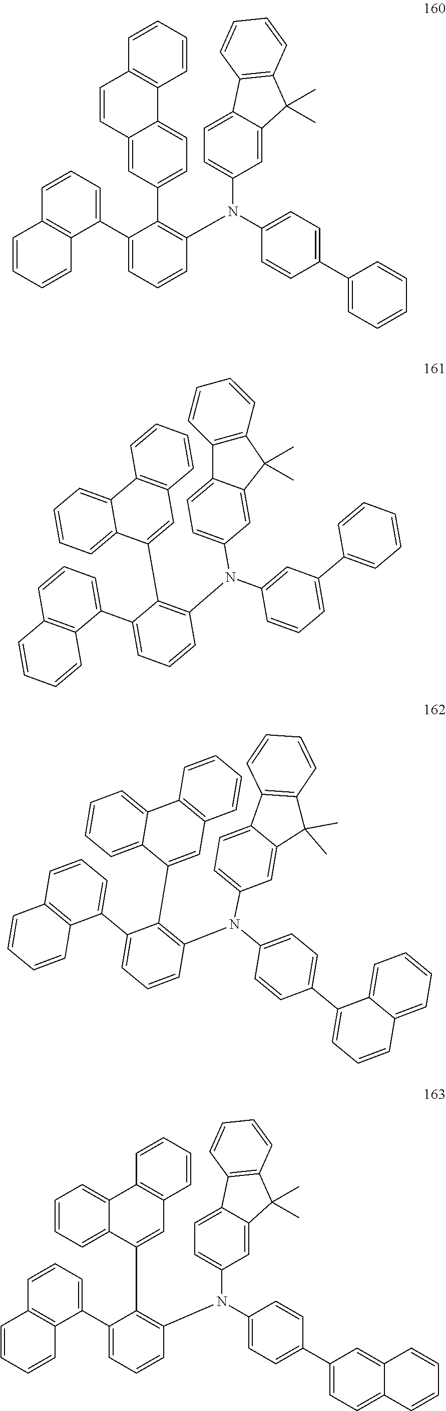 [SMILES: CC1(C)c2ccccc2-c2ccc(N(c3ccc(-c4ccccc4)cc3)c3cccc(-c4cccc5ccccc45)c3-c3ccc4c(ccc5ccccc54)c3)cc21 ; CC1(C)c2ccccc2-c2ccc(N(c3cccc(-c4ccccc4)c3)c3cccc(-c4cccc5ccccc45)c3-c3cc4ccccc4c4ccccc34)cc21 ; CC1(C)c2ccccc2-c2ccc(N(c3ccc(-c4cccc5ccccc45)cc3)c3cccc(-c4cccc5ccccc45)c3-c3cc4ccccc4c4ccccc34)cc21 ; CC1(C)c2ccccc2-c2ccc(N(c3ccc(-c4ccc5ccccc5c4)cc3)c3cccc(-c4cccc5ccccc45)c3-c3cc4ccccc4c4ccccc34)cc21]

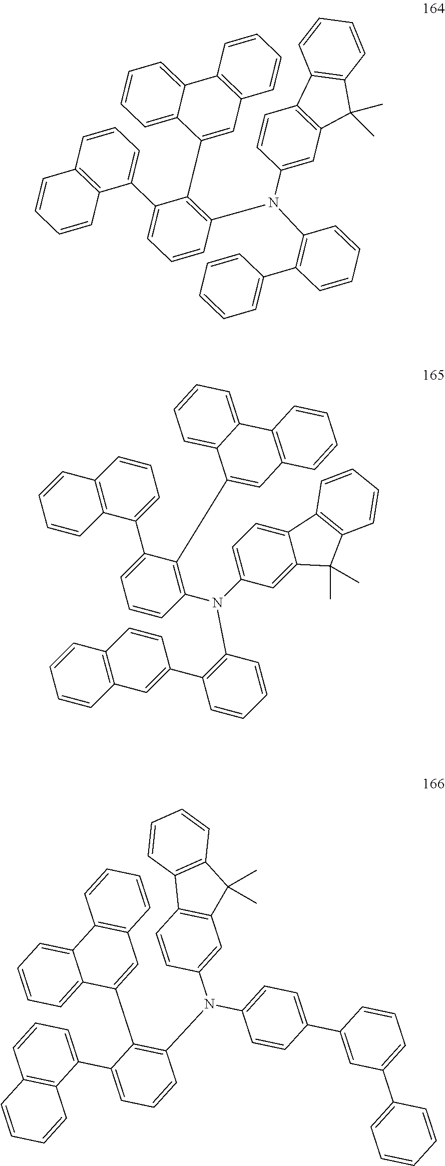 [SMILES: CC1(C)c2ccccc2-c2ccc(N(c3ccccc3-c3ccccc3)c3cccc(-c4cccc5ccccc45)c3-c3cc4ccccc4c4ccccc34)cc21 ; CC1(C)c2ccccc2-c2ccc(N(c3ccccc3-c3ccc4ccccc4c3)c3cccc(-c4cccc5ccccc45)c3-c3cc4ccccc4c4ccccc34)cc21 ; CC1(C)c2ccccc2-c2ccc(N(c3ccc(-c4cccc(-c5ccccc5)c4)cc3)c3cccc(-c4cccc5ccccc45)c3-c3cc4ccccc4c4ccccc34)cc21]

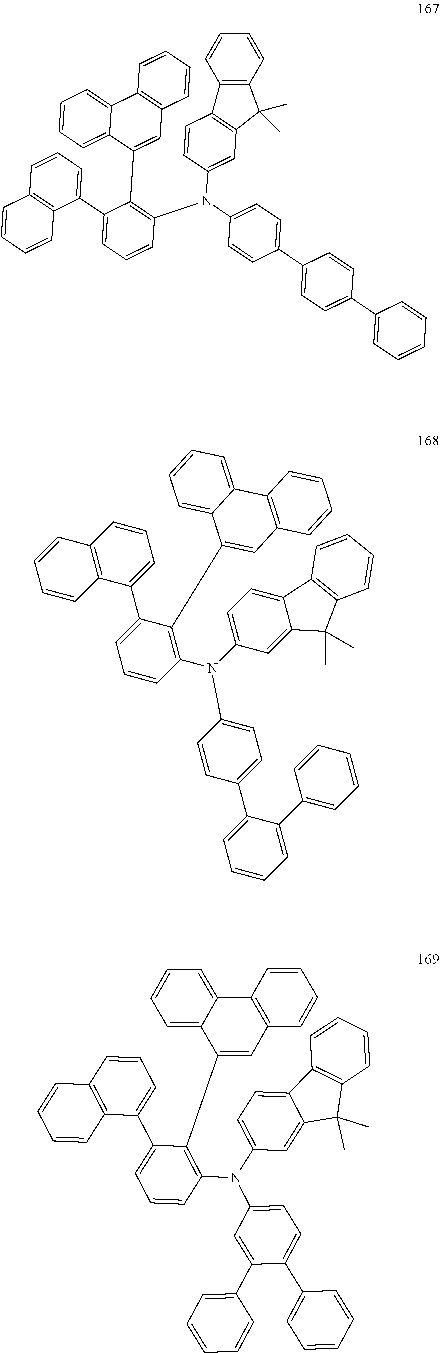 [SMILES: CC1(C)c2ccccc2-c2ccc(N(c3ccc(-c4ccc(-c5ccccc5)cc4)cc3)c3cccc(-c4cccc5ccccc45)c3-c3cc4ccccc4c4ccccc34)cc21 ; CC1(C)c2ccccc2-c2ccc(N(c3ccc(-c4ccccc4-c4ccccc4)cc3)c3cccc(-c4cccc5ccccc45)c3-c3cc4ccccc4c4ccccc34)cc21 ; CC1(C)c2ccccc2-c2ccc(N(c3ccc(-c4ccccc4)c(-c4ccccc4)c3)c3cccc(-c4cccc5ccccc45)c3-c3cc4ccccc4c4ccccc34)cc21]

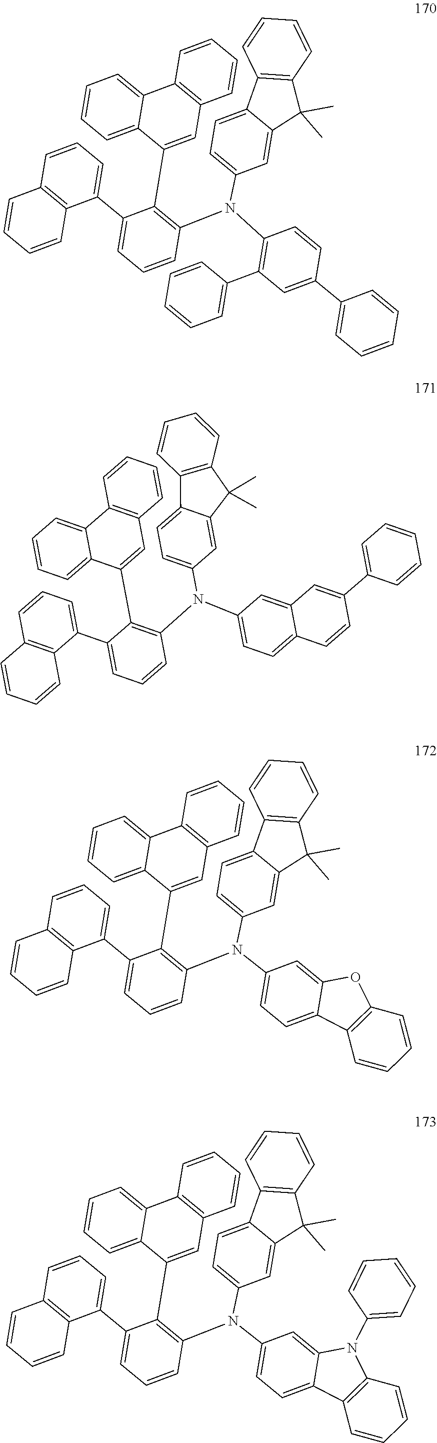 [SMILES: CC1(C)c2ccccc2-c2ccc(N(c3ccc(-c4ccccc4)cc3-c3ccccc3)c3cccc(-c4cccc5ccccc45)c3-c3cc4ccccc4c4ccccc34)cc21 ; CC1(C)c2ccccc2-c2ccc(N(c3ccc4ccc(-c5ccccc5)cc4c3)c3cccc(-c4cccc5ccccc45)c3-c3cc4ccccc4c4ccccc34)cc21 ; CC1(C)c2ccccc2-c2ccc(N(c3ccc4c(c3)oc3ccccc34)c3cccc(-c4cccc5ccccc45)c3-c3cc4ccccc4c4ccccc34)cc21 ; CC1(C)c2ccccc2-c2ccc(N(c3ccc4c5ccccc5n(-c5ccccc5)c4c3)c3cccc(-c4cccc5ccccc45)c3-c3cc4ccccc4c4ccccc34)cc21]

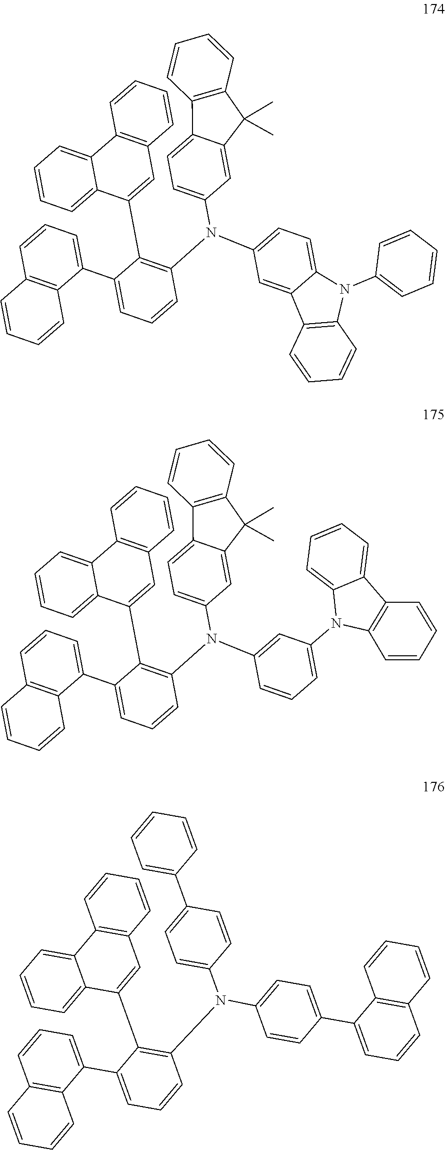 [SMILES: CC1(C)c2ccccc2-c2ccc(N(c3ccc4c(c3)c3ccccc3n4-c3ccccc3)c3cccc(-c4cccc5ccccc45)c3-c3cc4ccccc4c4ccccc34)cc21 ; CC1(C)c2ccccc2-c2ccc(N(c3cccc(-n4c5ccccc5c5ccccc54)c3)c3cccc(-c4cccc5ccccc45)c3-c3cc4ccccc4c4ccccc34)cc21 ; c1ccc(-c2ccc(N(c3ccc(-c4cccc5ccccc45)cc3)c3cccc(-c4cccc5ccccc45)c3-c3cc4ccccc4c4ccccc34)cc2)cc1]

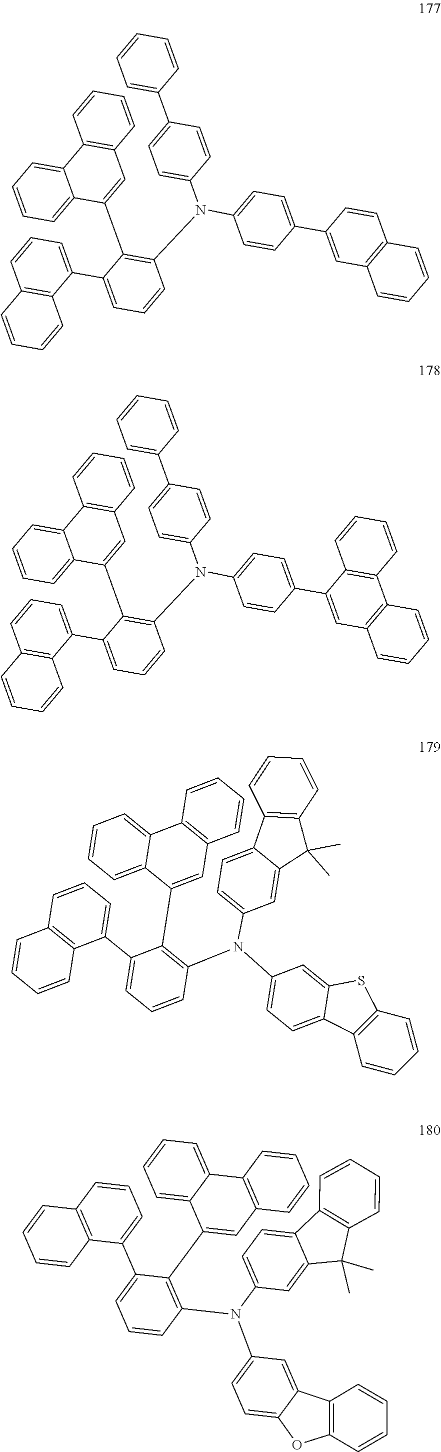 [SMILES: c1ccc(-c2ccc(N(c3ccc(-c4ccc5ccccc5c4)cc3)c3cccc(-c4cccc5ccccc45)c3-c3cc4ccccc4c4ccccc34)cc2)cc1 ; c1ccc(-c2ccc(N(c3ccc(-c4cc5ccccc5c5ccccc45)cc3)c3cccc(-c4cccc5ccccc45)c3-c3cc4ccccc4c4ccccc34)cc2)cc1 ; CC1(C)c2ccccc2-c2ccc(N(c3ccc4c(c3)sc3ccccc34)c3cccc(-c4cccc5ccccc45)c3-c3cc4ccccc4c4ccccc34)cc21 ; CC1(C)c2ccccc2-c2ccc(N(c3ccc4oc5ccccc5c4c3)c3cccc(-c4cccc5ccccc45)c3-c3cc4ccccc4c4ccccc34)cc21]

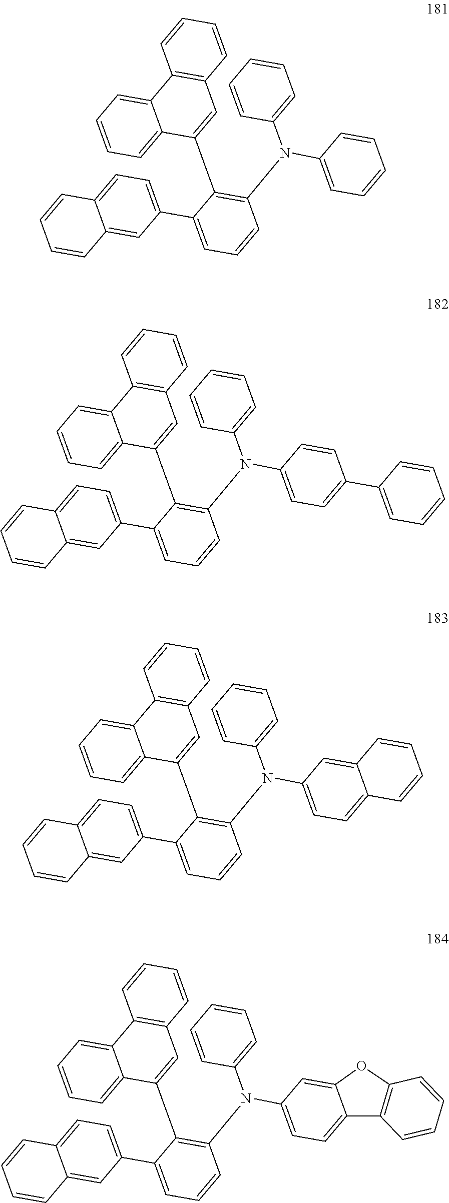 [SMILES: c1ccc(N(c2ccccc2)c2cccc(-c3ccc4ccccc4c3)c2-c2cc3ccccc3c3ccccc23)cc1 ; c1ccc(-c2ccc(N(c3ccccc3)c3cccc(-c4ccc5ccccc5c4)c3-c3cc4ccccc4c4ccccc34)cc2)cc1 ; c1ccc(N(c2ccc3ccccc3c2)c2cccc(-c3ccc4ccccc4c3)c2-c2cc3ccccc3c3ccccc23)cc1 ; c1ccc(N(c2ccc3c(c2)oc2ccccc23)c2cccc(-c3ccc4ccccc4c3)c2-c2cc3ccccc3c3ccccc23)cc1]

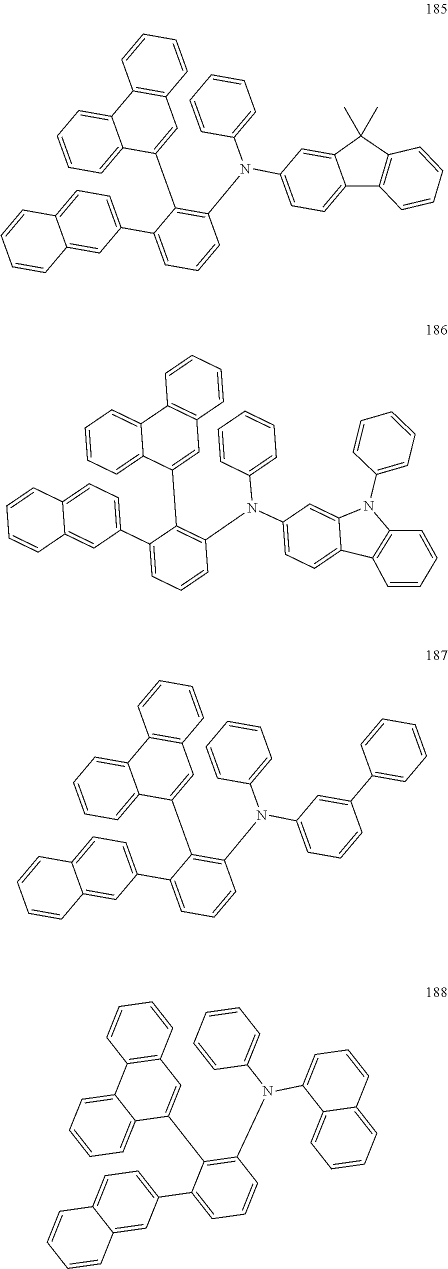 [SMILES: CC1(C)c2ccccc2-c2ccc(N(c3ccccc3)c3cccc(-c4ccc5ccccc5c4)c3-c3cc4ccccc4c4ccccc34)cc21 ; c1ccc(N(c2ccc3c4ccccc4n(-c4ccccc4)c3c2)c2cccc(-c3ccc4ccccc4c3)c2-c2cc3ccccc3c3ccccc23)cc1 ; c1ccc(-c2cccc(N(c3ccccc3)c3cccc(-c4ccc5ccccc5c4)c3-c3cc4ccccc4c4ccccc34)c2)cc1 ; c1ccc(N(c2cccc(-c3ccc4ccccc4c3)c2-c2cc3ccccc3c3ccccc23)c2cccc3ccccc23)cc1]

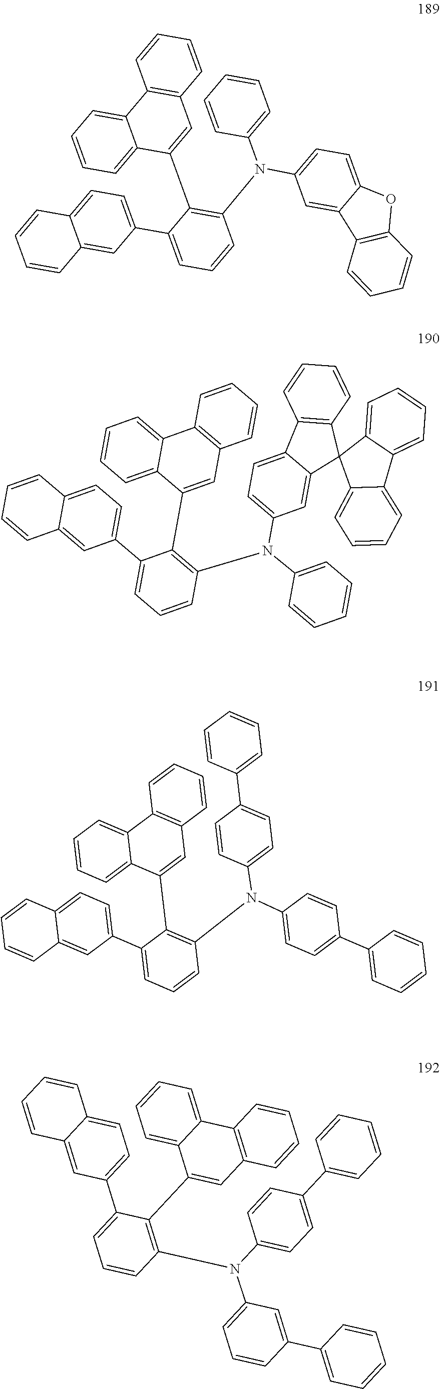 [SMILES: c1ccc(N(c2ccc3oc4ccccc4c3c2)c2cccc(-c3ccc4ccccc4c3)c2-c2cc3ccccc3c3ccccc23)cc1 ; c1ccc(N(c2ccc3c(c2)C2(c4ccccc4-c4ccccc42)c2ccccc2-3)c2cccc(-c3ccc4ccccc4c3)c2-c2cc3ccccc3c3ccccc23)cc1 ; c1ccc(-c2ccc(N(c3ccc(-c4ccccc4)cc3)c3cccc(-c4ccc5ccccc5c4)c3-c3cc4ccccc4c4ccccc34)cc2)cc1 ; c1ccc(-c2ccc(N(c3cccc(-c4ccccc4)c3)c3cccc(-c4ccc5ccccc5c4)c3-c3cc4ccccc4c4ccccc34)cc2)cc1]

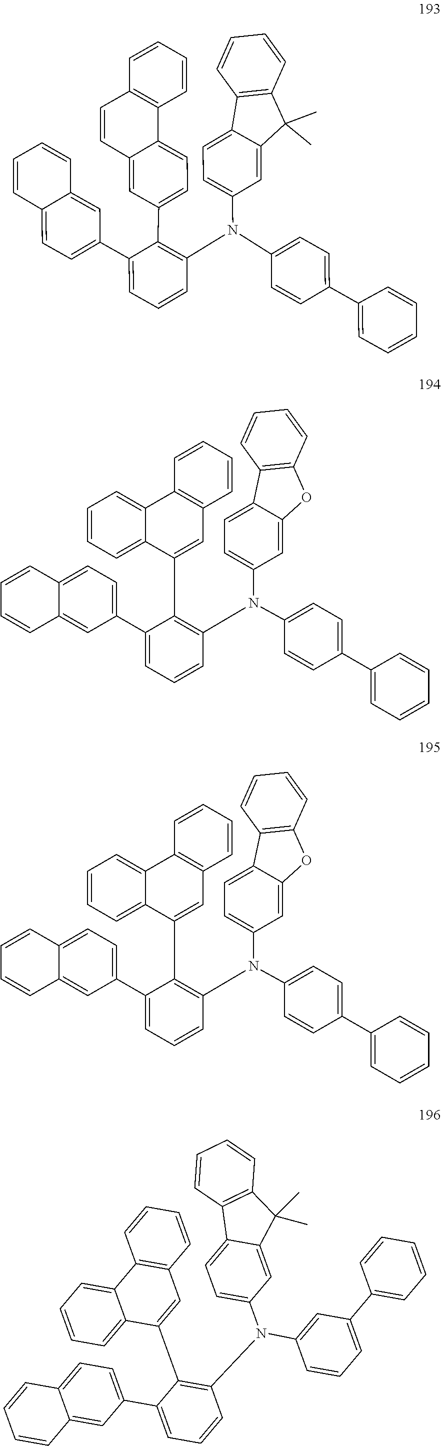 [SMILES: CC1(C)c2ccccc2-c2ccc(N(c3ccc(-c4ccccc4)cc3)c3cccc(-c4ccc5ccccc5c4)c3-c3ccc4c(ccc5ccccc54)c3)cc21 ; c1ccc(-c2ccc(N(c3ccc4c(c3)oc3ccccc34)c3cccc(-c4ccc5ccccc5c4)c3-c3cc4ccccc4c4ccccc34)cc2)cc1 ; c1ccc(-c2ccc(N(c3ccc4c(c3)oc3ccccc34)c3cccc(-c4ccc5ccccc5c4)c3-c3cc4ccccc4c4ccccc34)cc2)cc1 ; CC1(C)c2ccccc2-c2ccc(N(c3cccc(-c4ccccc4)c3)c3cccc(-c4ccc5ccccc5c4)c3-c3cc4ccccc4c4ccccc34)cc21]

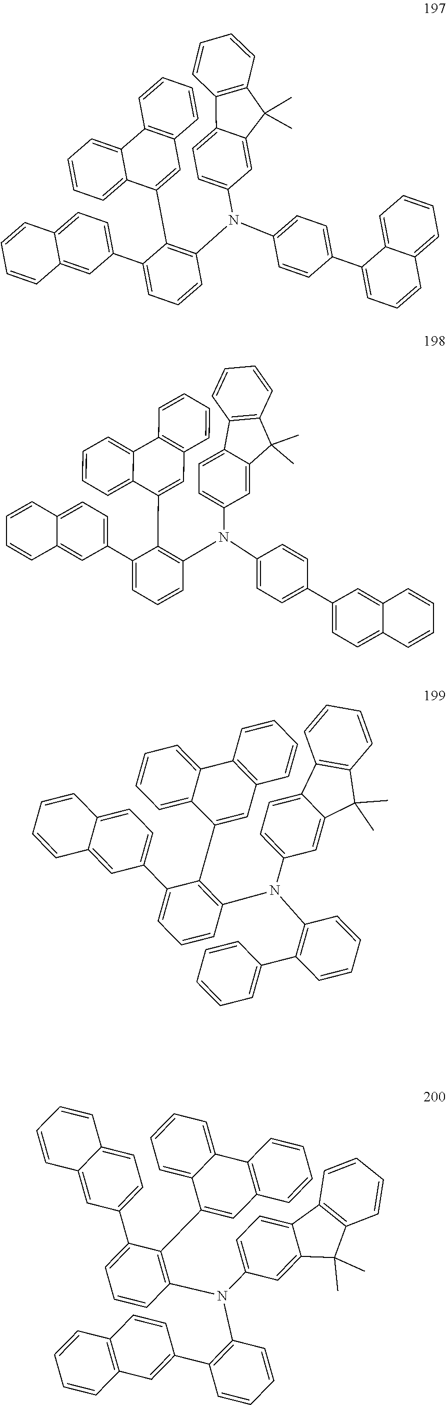 [SMILES: CC1(C)c2ccccc2-c2ccc(N(c3ccc(-c4cccc5ccccc45)cc3)c3cccc(-c4ccc5ccccc5c4)c3-c3cc4ccccc4c4ccccc34)cc21 ; CC1(C)c2ccccc2-c2ccc(N(c3ccc(-c4ccc5ccccc5c4)cc3)c3cccc(-c4ccc5ccccc5c4)c3-c3cc4ccccc4c4ccccc34)cc21 ; CC1(C)c2ccccc2-c2ccc(N(c3ccccc3-c3ccccc3)c3cccc(-c4ccc5ccccc5c4)c3-c3cc4ccccc4c4ccccc34)cc21 ; CC1(C)c2ccccc2-c2ccc(N(c3ccccc3-c3ccc4ccccc4c3)c3cccc(-c4ccc5ccccc5c4)c3-c3cc4ccccc4c4ccccc34)cc21]

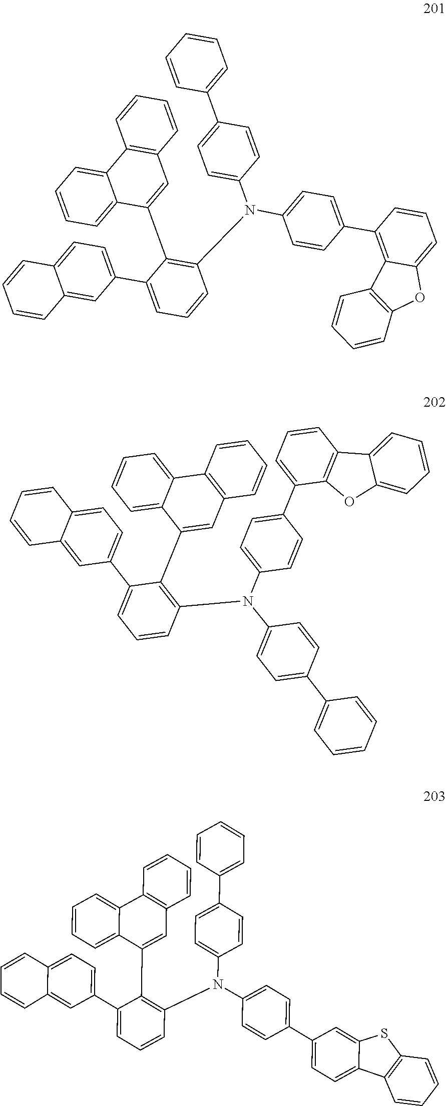 [SMILES: c1ccc(-c2ccc(N(c3ccc(-c4cccc5oc6ccccc6c45)cc3)c3cccc(-c4ccc5ccccc5c4)c3-c3cc4ccccc4c4ccccc34)cc2)cc1 ; c1ccc(-c2ccc(N(c3ccc(-c4cccc5c4oc4ccccc45)cc3)c3cccc(-c4ccc5ccccc5c4)c3-c3cc4ccccc4c4ccccc34)cc2)cc1 ; c1ccc(-c2ccc(N(c3ccc(-c4ccc5c(c4)sc4ccccc45)cc3)c3cccc(-c4ccc5ccccc5c4)c3-c3cc4ccccc4c4ccccc34)cc2)cc1]

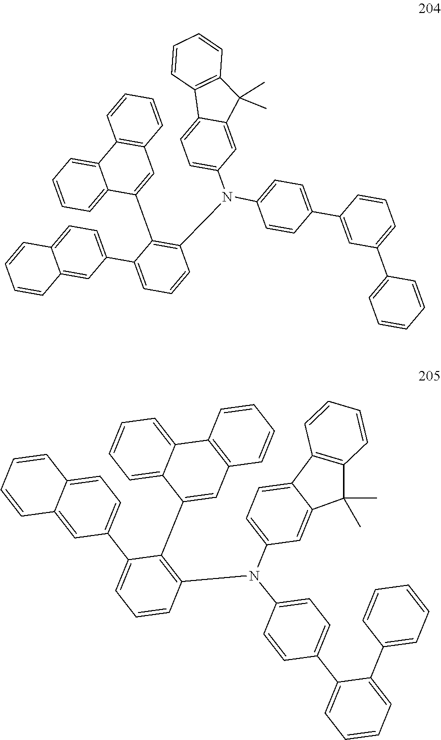 [SMILES: CC1(C)c2ccccc2-c2ccc(N(c3ccc(-c4cccc(-c5ccccc5)c4)cc3)c3cccc(-c4ccc5ccccc5c4)c3-c3cc4ccccc4c4ccccc34)cc21 ; CC1(C)c2ccccc2-c2ccc(N(c3ccc(-c4ccccc4-c4ccccc4)cc3)c3cccc(-c4ccc5ccccc5c4)c3-c3cc4ccccc4c4ccccc34)cc21]

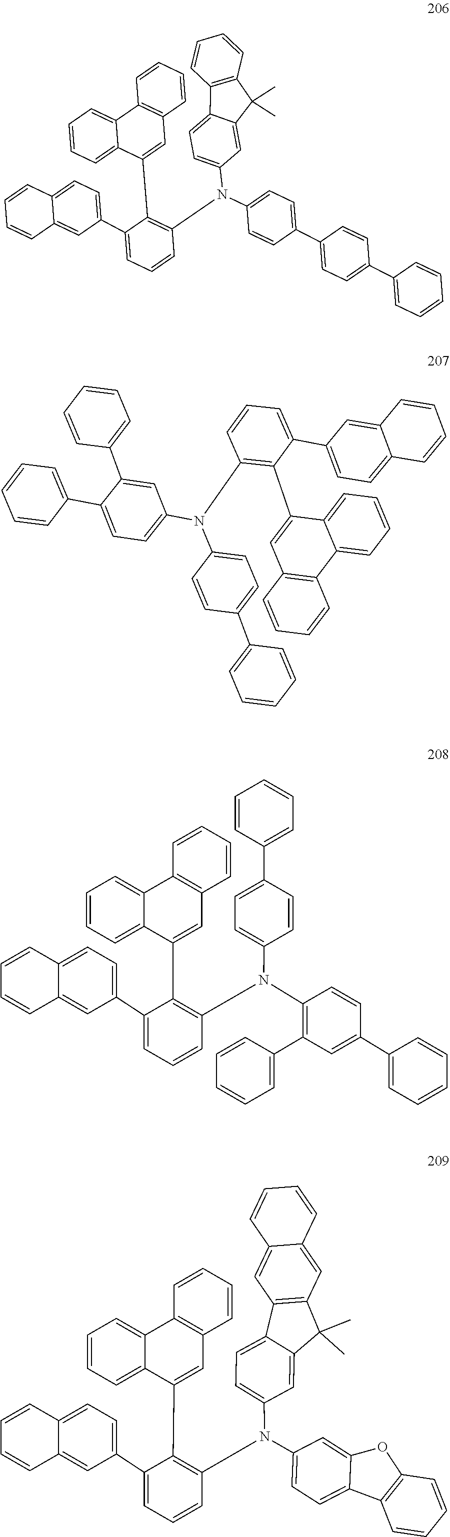 [SMILES: CC1(C)c2ccccc2-c2ccc(N(c3ccc(-c4ccc(-c5ccccc5)cc4)cc3)c3cccc(-c4ccc5ccccc5c4)c3-c3cc4ccccc4c4ccccc34)cc21 ; c1ccc(-c2ccc(N(c3ccc(-c4ccccc4)c(-c4ccccc4)c3)c3cccc(-c4ccc5ccccc5c4)c3-c3cc4ccccc4c4ccccc34)cc2)cc1 ; c1ccc(-c2ccc(N(c3ccc(-c4ccccc4)cc3-c3ccccc3)c3cccc(-c4ccc5ccccc5c4)c3-c3cc4ccccc4c4ccccc34)cc2)cc1 ; CC1(C)c2cc(N(c3ccc4c(c3)oc3ccccc34)c3cccc(-c4ccc5ccccc5c4)c3-c3cc4ccccc4c4ccccc34)ccc2-c2cc3ccccc3cc21]

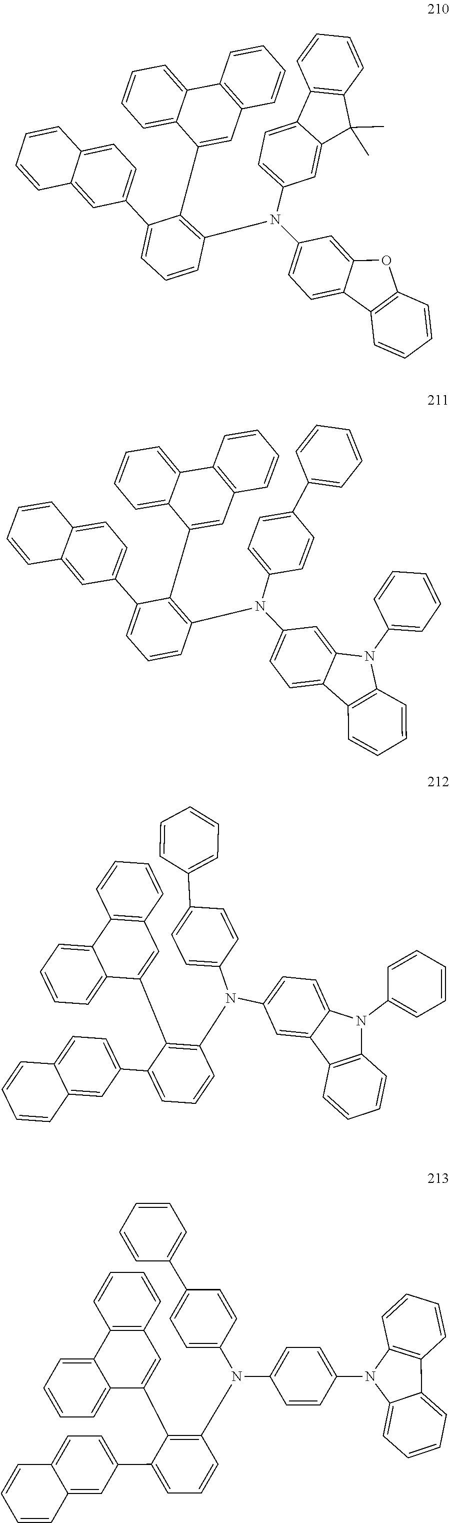 [SMILES: CC1(C)c2ccccc2-c2ccc(N(c3ccc4c(c3)oc3ccccc34)c3cccc(-c4ccc5ccccc5c4)c3-c3cc4ccccc4c4ccccc34)cc21 ; c1ccc(-c2ccc(N(c3ccc4c5ccccc5n(-c5ccccc5)c4c3)c3cccc(-c4ccc5ccccc5c4)c3-c3cc4ccccc4c4ccccc34)cc2)cc1 ; c1ccc(-c2ccc(N(c3ccc4c(c3)c3ccccc3n4-c3ccccc3)c3cccc(-c4ccc5ccccc5c4)c3-c3cc4ccccc4c4ccccc34)cc2)cc1 ; c1ccc(-c2ccc(N(c3ccc(-n4c5ccccc5c5ccccc54)cc3)c3cccc(-c4ccc5ccccc5c4)c3-c3cc4ccccc4c4ccccc34)cc2)cc1]

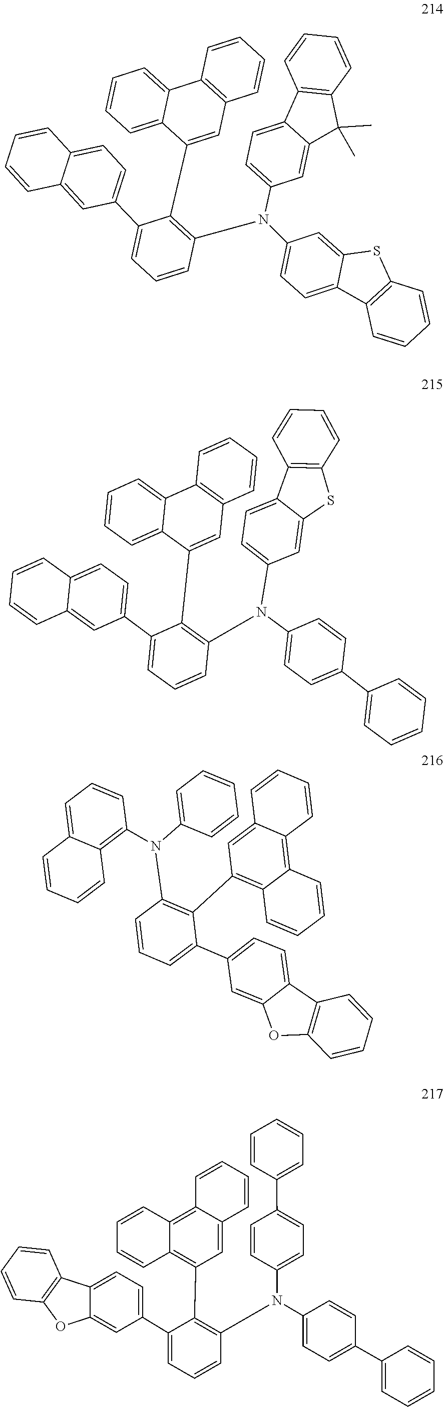 [SMILES: CC1(C)c2ccccc2-c2ccc(N(c3ccc4c(c3)sc3ccccc34)c3cccc(-c4ccc5ccccc5c4)c3-c3cc4ccccc4c4ccccc34)cc21 ; c1ccc(-c2ccc(N(c3ccc4c(c3)sc3ccccc34)c3cccc(-c4ccc5ccccc5c4)c3-c3cc4ccccc4c4ccccc34)cc2)cc1 ; c1ccc(N(c2cccc(-c3ccc4c(c3)oc3ccccc34)c2-c2cc3ccccc3c3ccccc23)c2cccc3ccccc23)cc1 ; c1ccc(-c2ccc(N(c3ccc(-c4ccccc4)cc3)c3cccc(-c4ccc5c(c4)oc4ccccc45)c3-c3cc4ccccc4c4ccccc34)cc2)cc1]

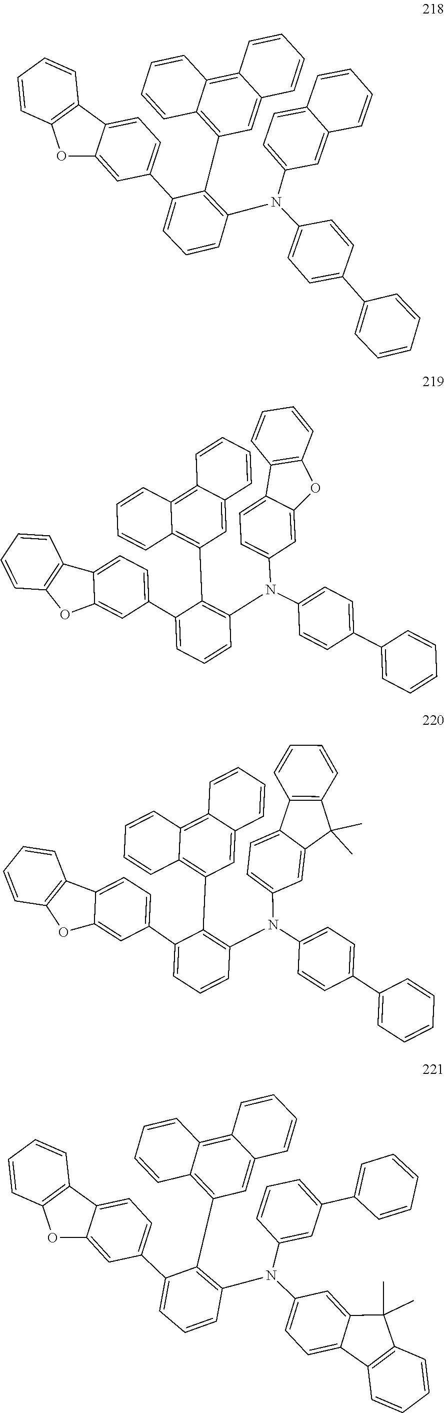 [SMILES: c1ccc(-c2ccc(N(c3ccc4ccccc4c3)c3cccc(-c4ccc5c(c4)oc4ccccc45)c3-c3cc4ccccc4c4ccccc34)cc2)cc1 ; c1ccc(-c2ccc(N(c3ccc4c(c3)oc3ccccc34)c3cccc(-c4ccc5c(c4)oc4ccccc45)c3-c3cc4ccccc4c4ccccc34)cc2)cc1 ; CC1(C)c2ccccc2-c2ccc(N(c3ccc(-c4ccccc4)cc3)c3cccc(-c4ccc5c(c4)oc4ccccc45)c3-c3cc4ccccc4c4ccccc34)cc21 ; CC1(C)c2ccccc2-c2ccc(N(c3cccc(-c4ccccc4)c3)c3cccc(-c4ccc5c(c4)oc4ccccc45)c3-c3cc4ccccc4c4ccccc34)cc21]

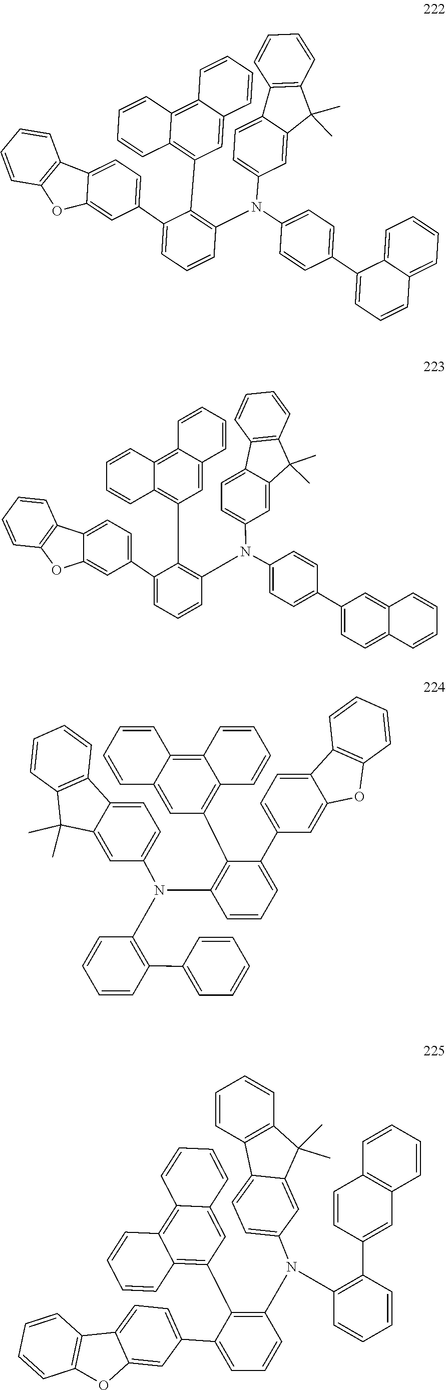 [SMILES: CC1(C)c2ccccc2-c2ccc(N(c3ccc(-c4cccc5ccccc45)cc3)c3cccc(-c4ccc5c(c4)oc4ccccc45)c3-c3cc4ccccc4c4ccccc34)cc21 ; CC1(C)c2ccccc2-c2ccc(N(c3ccc(-c4ccc5ccccc5c4)cc3)c3cccc(-c4ccc5c(c4)oc4ccccc45)c3-c3cc4ccccc4c4ccccc34)cc21 ; CC1(C)c2ccccc2-c2ccc(N(c3ccccc3-c3ccccc3)c3cccc(-c4ccc5c(c4)oc4ccccc45)c3-c3cc4ccccc4c4ccccc34)cc21 ; CC1(C)c2ccccc2-c2ccc(N(c3ccccc3-c3ccc4ccccc4c3)c3cccc(-c4ccc5c(c4)oc4ccccc45)c3-c3cc4ccccc4c4ccccc34)cc21]

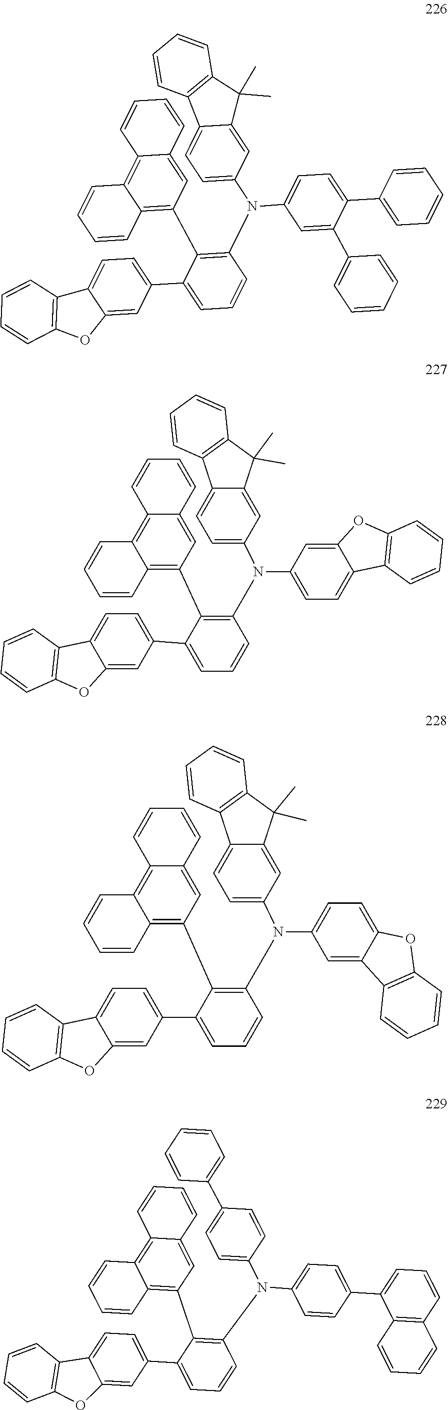 [SMILES: CC1(C)c2ccccc2-c2ccc(N(c3ccc(-c4ccccc4)c(-c4ccccc4)c3)c3cccc(-c4ccc5c(c4)oc4ccccc45)c3-c3cc4ccccc4c4ccccc34)cc21 ; CC1(C)c2ccccc2-c2ccc(N(c3ccc4c(c3)oc3ccccc34)c3cccc(-c4ccc5c(c4)oc4ccccc45)c3-c3cc4ccccc4c4ccccc34)cc21 ; CC1(C)c2ccccc2-c2ccc(N(c3ccc4oc5ccccc5c4c3)c3cccc(-c4ccc5c(c4)oc4ccccc45)c3-c3cc4ccccc4c4ccccc34)cc21 ; c1ccc(-c2ccc(N(c3ccc(-c4cccc5ccccc45)cc3)c3cccc(-c4ccc5c(c4)oc4ccccc45)c3-c3cc4ccccc4c4ccccc34)cc2)cc1]

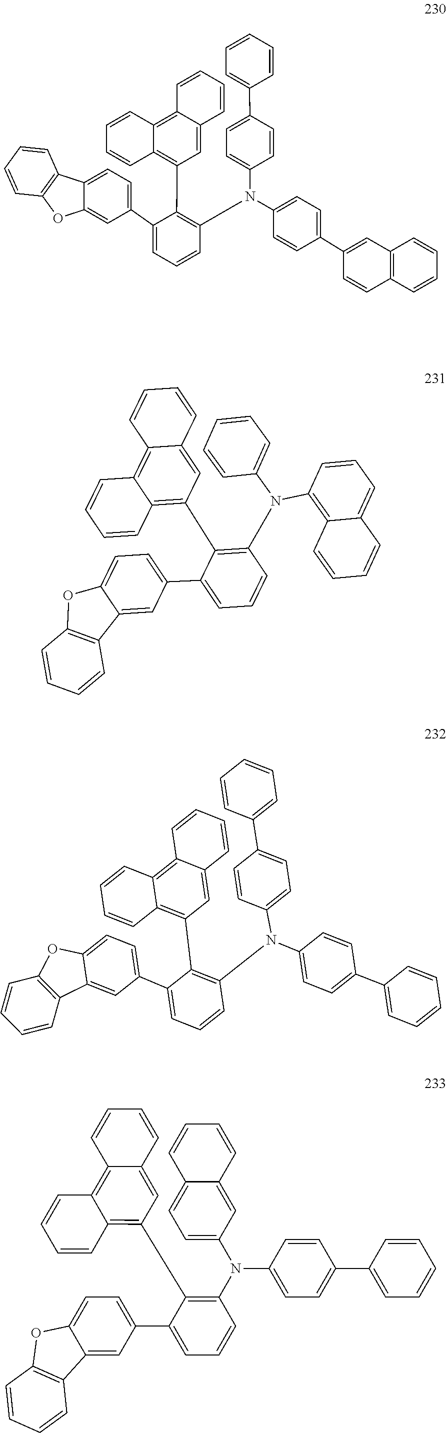 [SMILES: c1ccc(-c2ccc(N(c3ccc(-c4ccc5ccccc5c4)cc3)c3cccc(-c4ccc5c(c4)oc4ccccc45)c3-c3cc4ccccc4c4ccccc34)cc2)cc1 ; c1ccc(N(c2cccc(-c3ccc4oc5ccccc5c4c3)c2-c2cc3ccccc3c3ccccc23)c2cccc3ccccc23)cc1 ; c1ccc(-c2ccc(N(c3ccc(-c4ccccc4)cc3)c3cccc(-c4ccc5oc6ccccc6c5c4)c3-c3cc4ccccc4c4ccccc34)cc2)cc1 ; c1ccc(-c2ccc(N(c3ccc4ccccc4c3)c3cccc(-c4ccc5oc6ccccc6c5c4)c3-c3cc4ccccc4c4ccccc34)cc2)cc1]

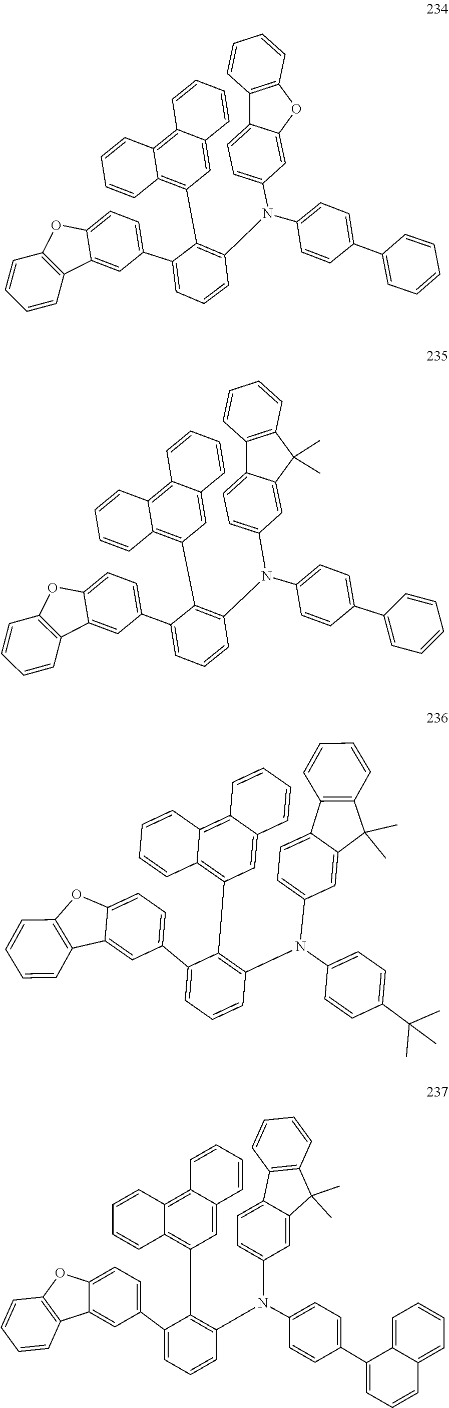 [SMILES: c1ccc(-c2ccc(N(c3ccc4c(c3)oc3ccccc34)c3cccc(-c4ccc5oc6ccccc6c5c4)c3-c3cc4ccccc4c4ccccc34)cc2)cc1 ; CC1(C)c2ccccc2-c2ccc(N(c3ccc(-c4ccccc4)cc3)c3cccc(-c4ccc5oc6ccccc6c5c4)c3-c3cc4ccccc4c4ccccc34)cc21 ; CC(C)(C)c1ccc(N(c2ccc3c(c2)C(C)(C)c2ccccc2-3)c2cccc(-c3ccc4oc5ccccc5c4c3)c2-c2cc3ccccc3c3ccccc23)cc1 ; CC1(C)c2ccccc2-c2ccc(N(c3ccc(-c4cccc5ccccc45)cc3)c3cccc(-c4ccc5oc6ccccc6c5c4)c3-c3cc4ccccc4c4ccccc34)cc21]

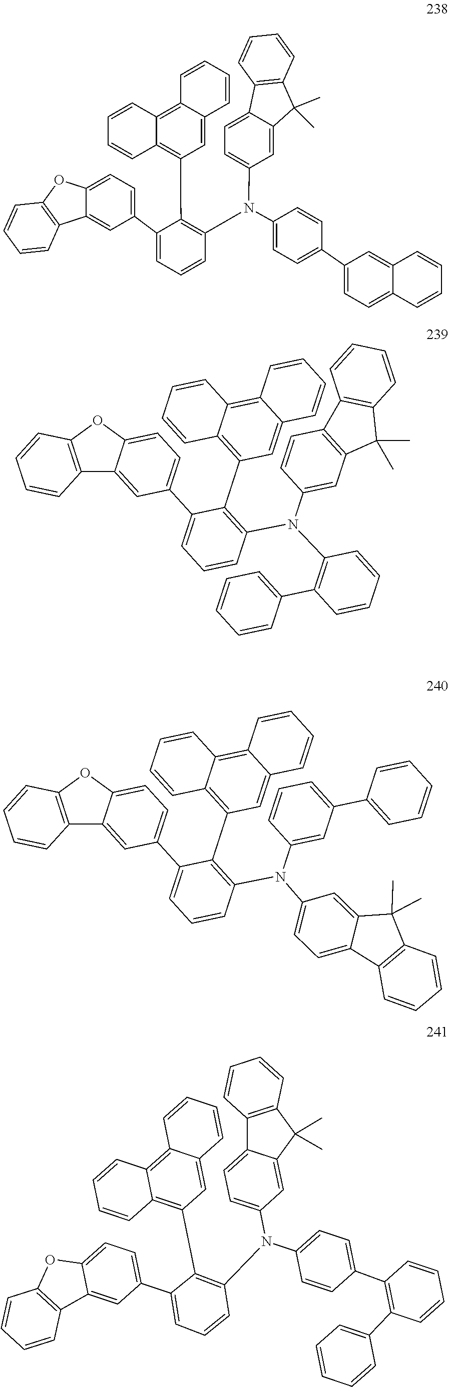

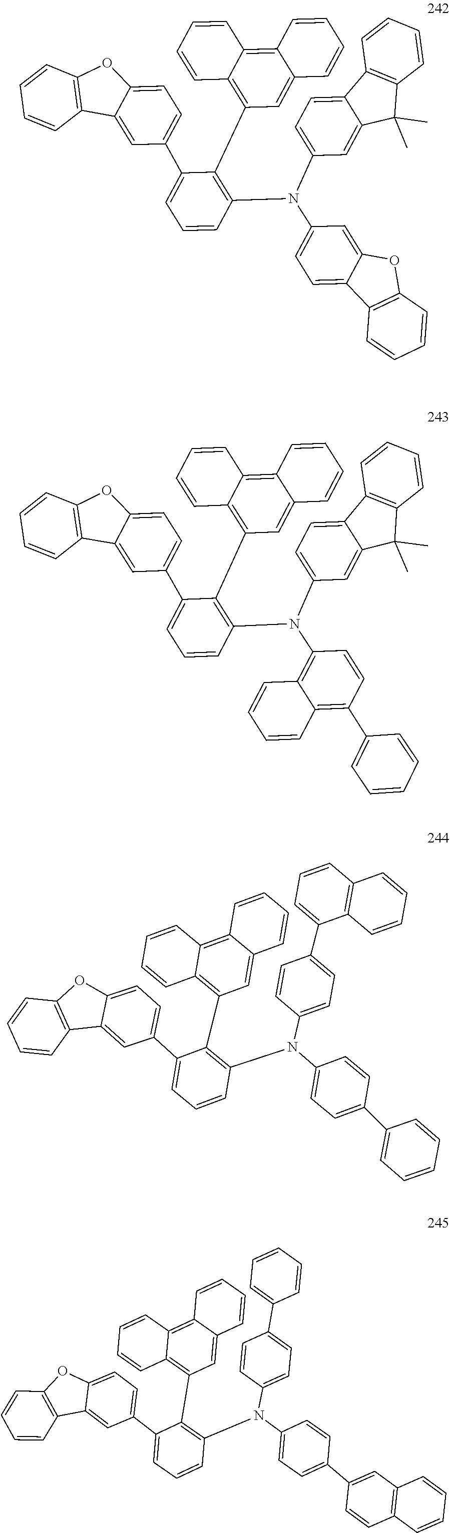 [SMILES: CC1(C)c2ccccc2-c2ccc(N(c3ccc4c(c3)oc3ccccc34)c3cccc(-c4ccc5oc6ccccc6c5c4)c3-c3cc4ccccc4c4ccccc34)cc21 ; CC1(C)c2ccccc2-c2ccc(N(c3cccc(-c4ccc5oc6ccccc6c5c4)c3-c3cc4ccccc4c4ccccc34)c3ccc(-c4ccccc4)c4ccccc34)cc21 ; c1ccc(-c2ccc(N(c3ccc(-c4cccc5ccccc45)cc3)c3cccc(-c4ccc5oc6ccccc6c5c4)c3-c3cc4ccccc4c4ccccc34)cc2)cc1 ; c1ccc(-c2ccc(N(c3ccc(-c4ccc5ccccc5c4)cc3)c3cccc(-c4ccc5oc6ccccc6c5c4)c3-c3cc4ccccc4c4ccccc34)cc2)cc1]

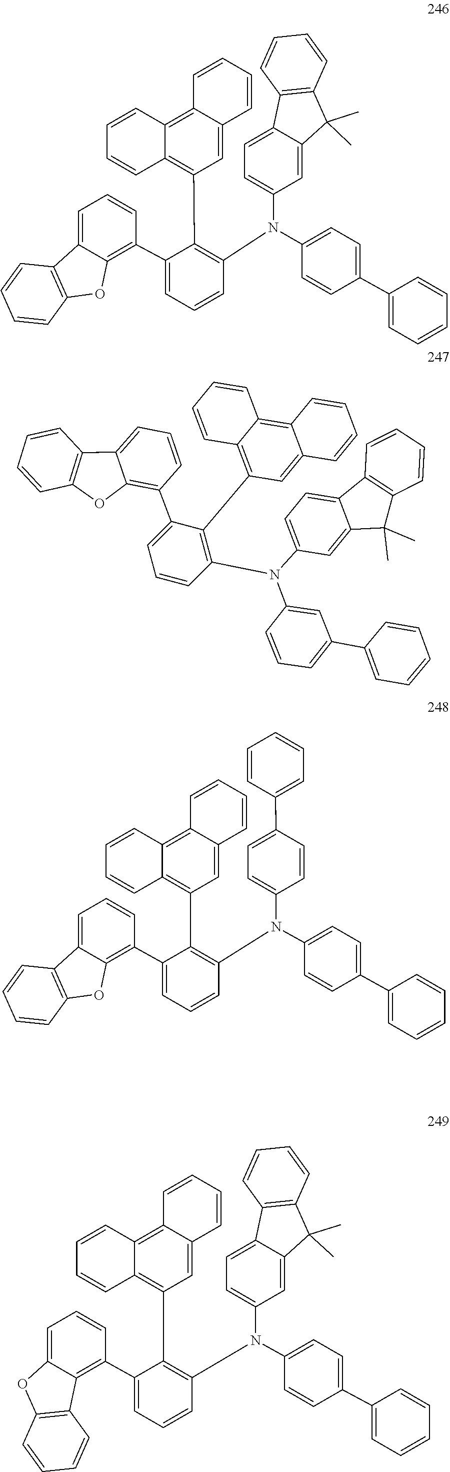 [SMILES: CC1(C)c2ccccc2-c2ccc(N(c3ccc(-c4ccccc4)cc3)c3cccc(-c4cccc5c4oc4ccccc45)c3-c3cc4ccccc4c4ccccc34)cc21 ; CC1(C)c2ccccc2-c2ccc(N(c3cccc(-c4ccccc4)c3)c3cccc(-c4cccc5c4oc4ccccc45)c3-c3cc4ccccc4c4ccccc34)cc21 ; c1ccc(-c2ccc(N(c3ccc(-c4ccccc4)cc3)c3cccc(-c4cccc5c4oc4ccccc45)c3-c3cc4ccccc4c4ccccc34)cc2)cc1 ; CC1(C)c2ccccc2-c2ccc(N(c3ccc(-c4ccccc4)cc3)c3cccc(-c4cccc5oc6ccccc6c45)c3-c3cc4ccccc4c4ccccc34)cc21]

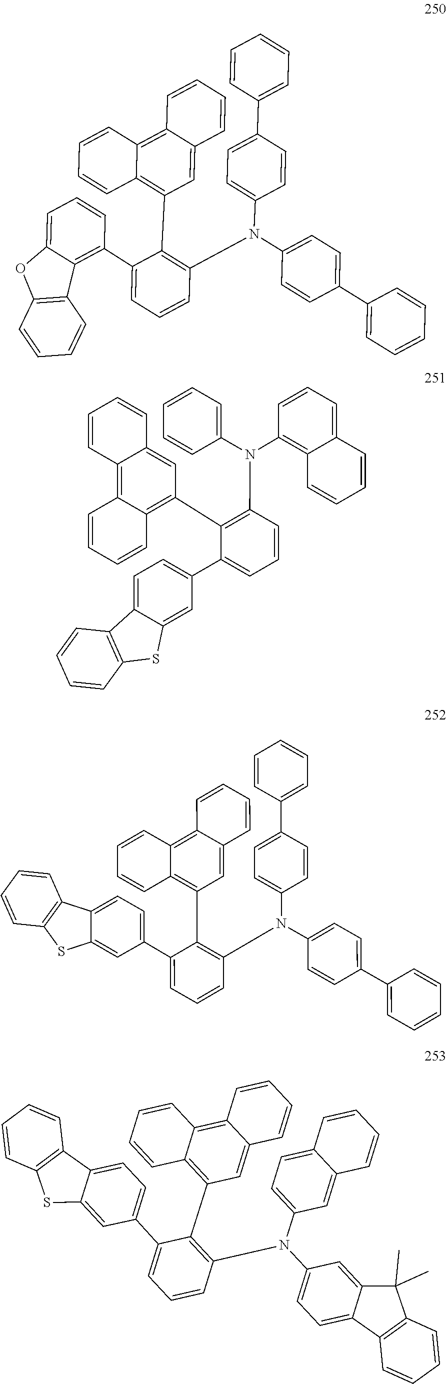 [SMILES: c1ccc(-c2ccc(N(c3ccc(-c4ccccc4)cc3)c3cccc(-c4cccc5oc6ccccc6c45)c3-c3cc4ccccc4c4ccccc34)cc2)cc1 ; c1ccc(N(c2cccc(-c3ccc4c(c3)sc3ccccc34)c2-c2cc3ccccc3c3ccccc23)c2cccc3ccccc23)cc1 ; c1ccc(-c2ccc(N(c3ccc(-c4ccccc4)cc3)c3cccc(-c4ccc5c(c4)sc4ccccc45)c3-c3cc4ccccc4c4ccccc34)cc2)cc1 ; CC1(C)c2ccccc2-c2ccc(N(c3ccc4ccccc4c3)c3cccc(-c4ccc5c(c4)sc4ccccc45)c3-c3cc4ccccc4c4ccccc34)cc21]

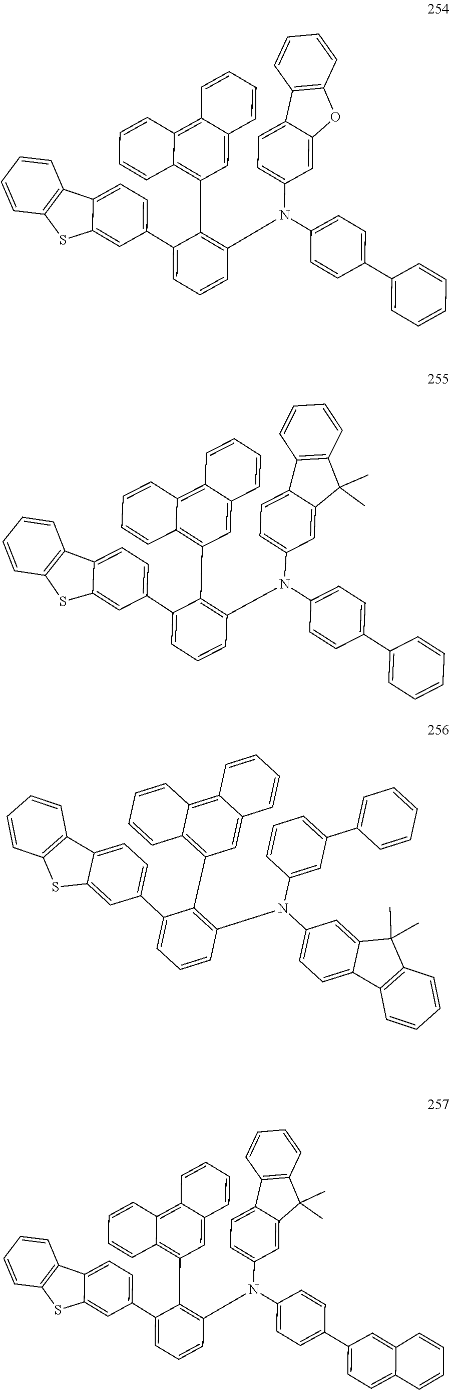 [SMILES: c1ccc(-c2ccc(N(c3ccc4c(c3)oc3ccccc34)c3cccc(-c4ccc5c(c4)sc4ccccc45)c3-c3cc4ccccc4c4ccccc34)cc2)cc1 ; CC1(C)c2ccccc2-c2ccc(N(c3ccc(-c4ccccc4)cc3)c3cccc(-c4ccc5c(c4)sc4ccccc45)c3-c3cc4ccccc4c4ccccc34)cc21 ; CC1(C)c2ccccc2-c2ccc(N(c3cccc(-c4ccccc4)c3)c3cccc(-c4ccc5c(c4)sc4ccccc45)c3-c3cc4ccccc4c4ccccc34)cc21 ; CC1(C)c2ccccc2-c2ccc(N(c3ccc(-c4ccc5ccccc5c4)cc3)c3cccc(-c4ccc5c(c4)sc4ccccc45)c3-c3cc4ccccc4c4ccccc34)cc21]

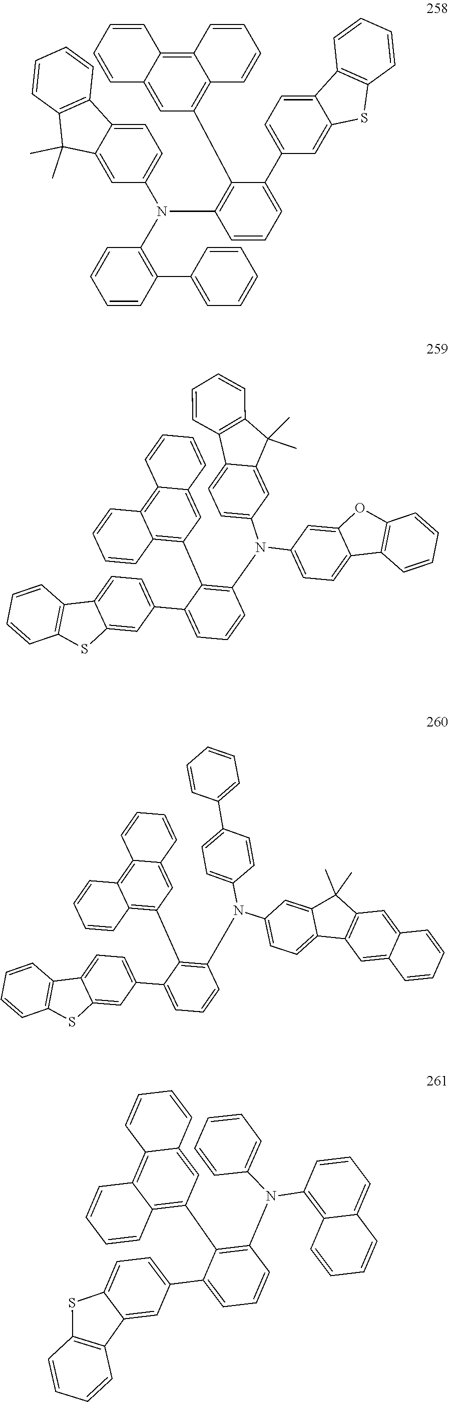 [SMILES: CC1(C)c2ccccc2-c2ccc(N(c3ccccc3-c3ccccc3)c3cccc(-c4ccc5c(c4)sc4ccccc45)c3-c3cc4ccccc4c4ccccc34)cc21 ; CC1(C)c2ccccc2-c2ccc(N(c3ccc4c(c3)oc3ccccc34)c3cccc(-c4ccc5c(c4)sc4ccccc45)c3-c3cc4ccccc4c4ccccc34)cc21 ; CC1(C)c2cc(N(c3ccc(-c4ccccc4)cc3)c3cccc(-c4ccc5c(c4)sc4ccccc45)c3-c3cc4ccccc4c4ccccc34)ccc2-c2cc3ccccc3cc21 ; c1ccc(N(c2cccc(-c3ccc4sc5ccccc5c4c3)c2-c2cc3ccccc3c3ccccc23)c2cccc3ccccc23)cc1]

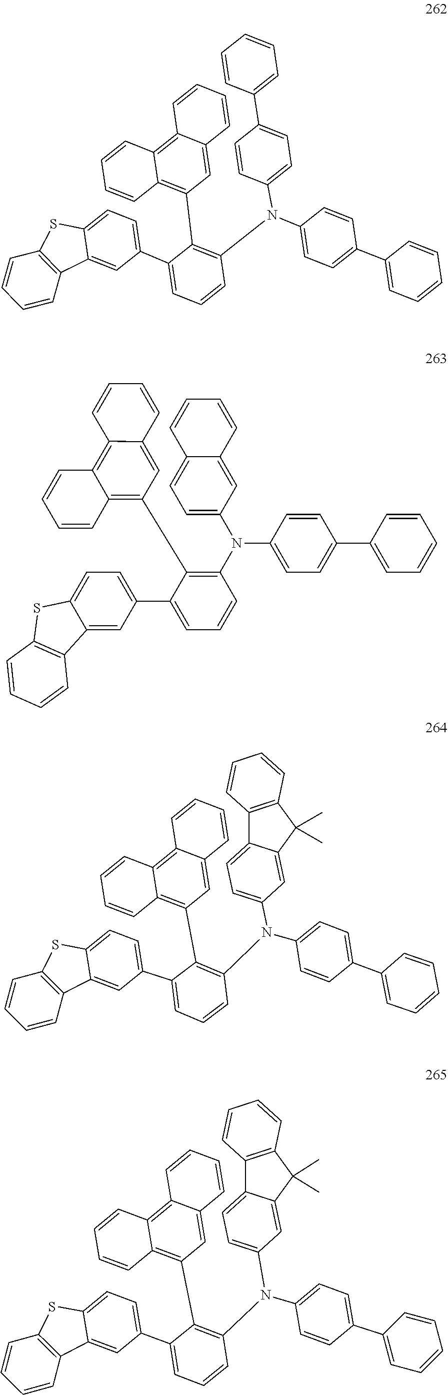 [SMILES: c1ccc(-c2ccc(N(c3ccc(-c4ccccc4)cc3)c3cccc(-c4ccc5sc6ccccc6c5c4)c3-c3cc4ccccc4c4ccccc34)cc2)cc1 ; c1ccc(-c2ccc(N(c3ccc4ccccc4c3)c3cccc(-c4ccc5sc6ccccc6c5c4)c3-c3cc4ccccc4c4ccccc34)cc2)cc1 ; CC1(C)c2ccccc2-c2ccc(N(c3ccc(-c4ccccc4)cc3)c3cccc(-c4ccc5sc6ccccc6c5c4)c3-c3cc4ccccc4c4ccccc34)cc21 ; CC1(C)c2ccccc2-c2ccc(N(c3ccc(-c4ccccc4)cc3)c3cccc(-c4ccc5sc6ccccc6c5c4)c3-c3cc4ccccc4c4ccccc34)cc21]

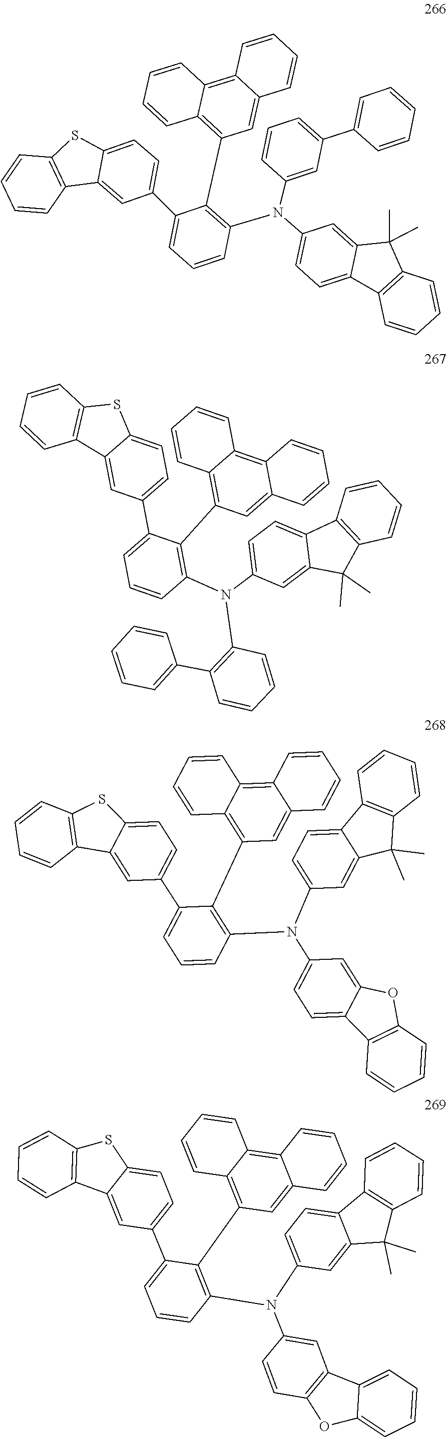 [SMILES: CC1(C)c2ccccc2-c2ccc(N(c3cccc(-c4ccccc4)c3)c3cccc(-c4ccc5sc6ccccc6c5c4)c3-c3cc4ccccc4c4ccccc34)cc21 ; CC1(C)c2ccccc2-c2ccc(N(c3ccccc3-c3ccccc3)c3cccc(-c4ccc5sc6ccccc6c5c4)c3-c3cc4ccccc4c4ccccc34)cc21 ; CC1(C)c2ccccc2-c2ccc(N(c3ccc4c(c3)oc3ccccc34)c3cccc(-c4ccc5sc6ccccc6c5c4)c3-c3cc4ccccc4c4ccccc34)cc21 ; CC1(C)c2ccccc2-c2ccc(N(c3ccc4oc5ccccc5c4c3)c3cccc(-c4ccc5sc6ccccc6c5c4)c3-c3cc4ccccc4c4ccccc34)cc21]

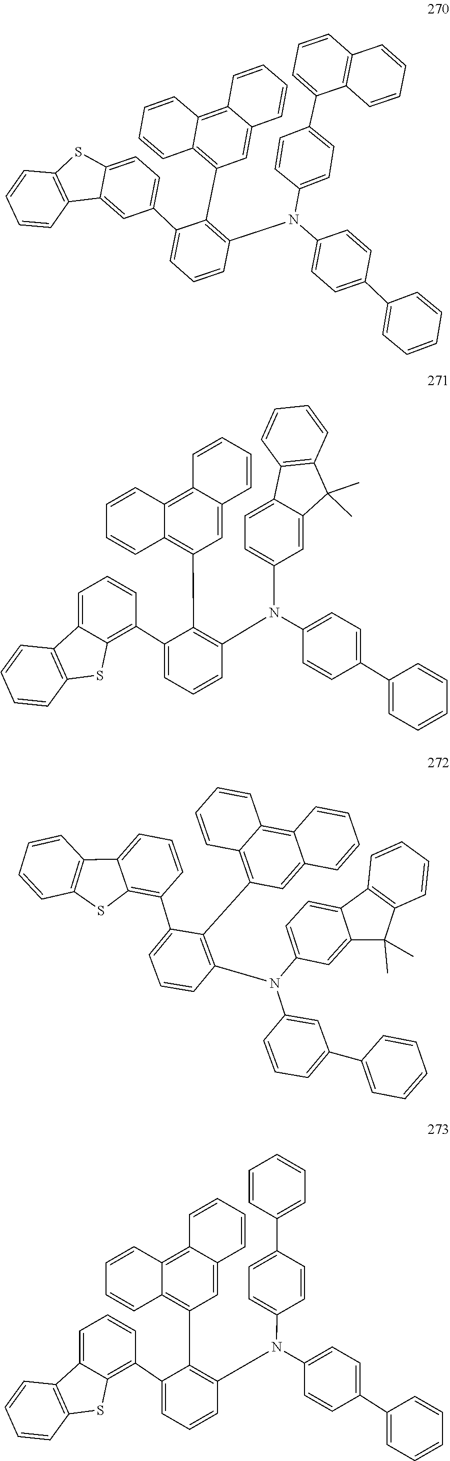 [SMILES: c1ccc(-c2ccc(N(c3ccc(-c4cccc5ccccc45)cc3)c3cccc(-c4ccc5sc6ccccc6c5c4)c3-c3cc4ccccc4c4ccccc34)cc2)cc1 ; CC1(C)c2ccccc2-c2ccc(N(c3ccc(-c4ccccc4)cc3)c3cccc(-c4cccc5c4sc4ccccc45)c3-c3cc4ccccc4c4ccccc34)cc21 ; CC1(C)c2ccccc2-c2ccc(N(c3cccc(-c4ccccc4)c3)c3cccc(-c4cccc5c4sc4ccccc45)c3-c3cc4ccccc4c4ccccc34)cc21 ; c1ccc(-c2ccc(N(c3ccc(-c4ccccc4)cc3)c3cccc(-c4cccc5c4sc4ccccc45)c3-c3cc4ccccc4c4ccccc34)cc2)cc1]

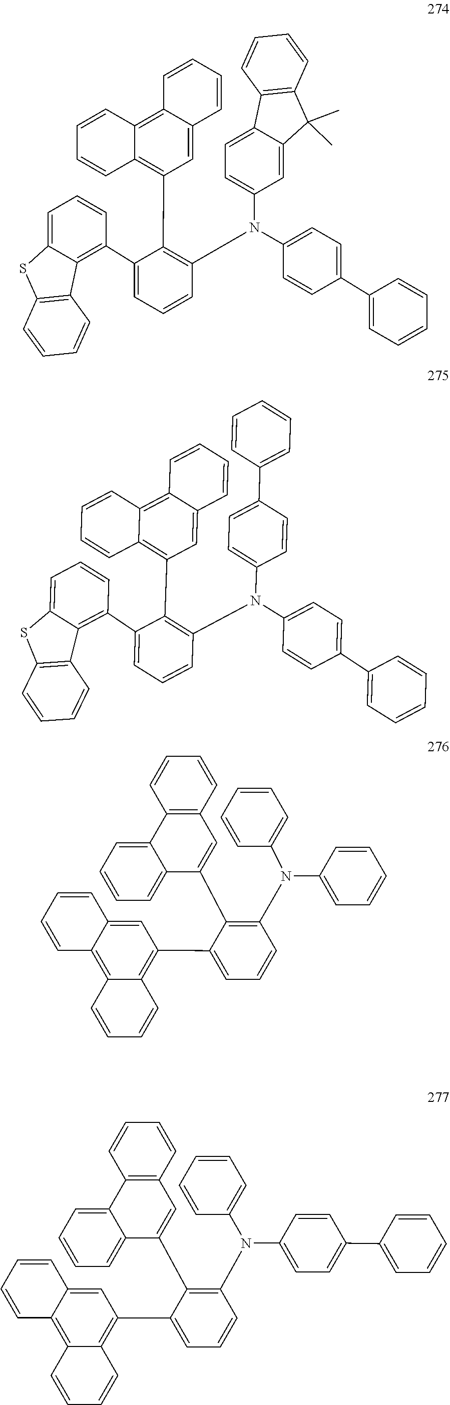 [SMILES: CC1(C)c2ccccc2-c2ccc(N(c3ccc(-c4ccccc4)cc3)c3cccc(-c4cccc5sc6ccccc6c45)c3-c3cc4ccccc4c4ccccc34)cc21 ; c1ccc(-c2ccc(N(c3ccc(-c4ccccc4)cc3)c3cccc(-c4cccc5sc6ccccc6c45)c3-c3cc4ccccc4c4ccccc34)cc2)cc1 ; c1ccc(N(c2ccccc2)c2cccc(-c3cc4ccccc4c4ccccc34)c2-c2cc3ccccc3c3ccccc23)cc1 ; c1ccc(-c2ccc(N(c3ccccc3)c3cccc(-c4cc5ccccc5c5ccccc45)c3-c3cc4ccccc4c4ccccc34)cc2)cc1]

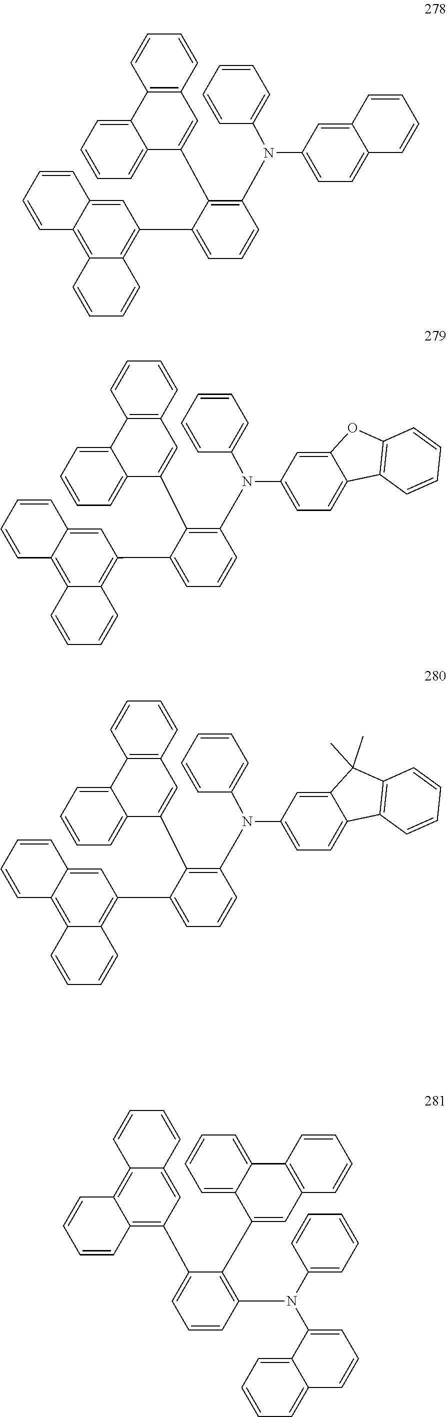

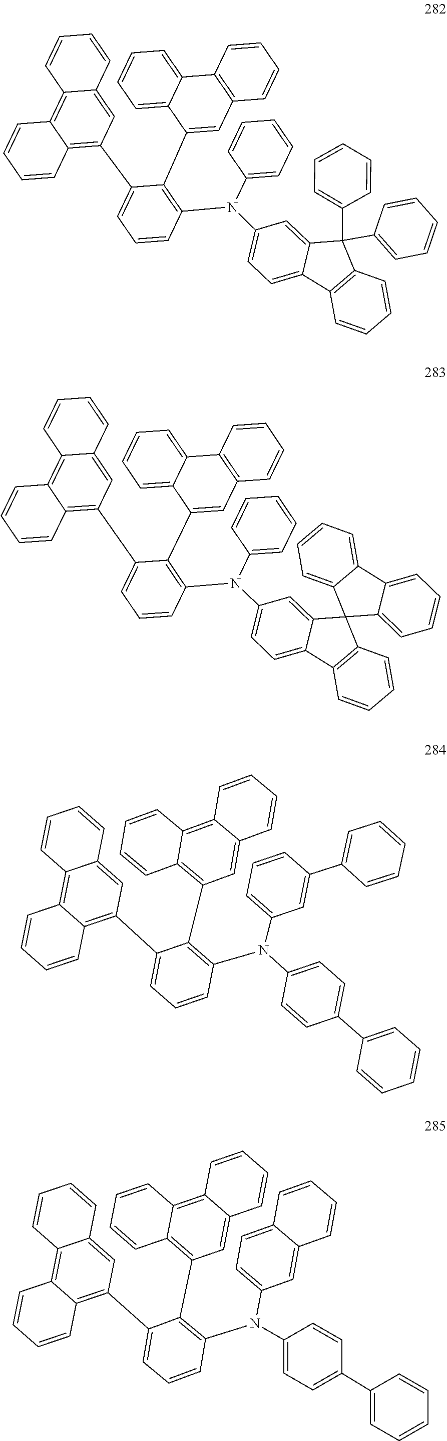 [SMILES: c1ccc(N(c2ccc3c(c2)C(c2ccccc2)(c2ccccc2)c2ccccc2-3)c2cccc(-c3cc4ccccc4c4ccccc34)c2-c2cc3ccccc3c3ccccc23)cc1 ; c1ccc(N(c2ccc3c(c2)C2(c4ccccc4-c4ccccc42)c2ccccc2-3)c2cccc(-c3cc4ccccc4c4ccccc34)c2-c2cc3ccccc3c3ccccc23)cc1 ; c1ccc(-c2ccc(N(c3cccc(-c4ccccc4)c3)c3cccc(-c4cc5ccccc5c5ccccc45)c3-c3cc4ccccc4c4ccccc34)cc2)cc1 ; c1ccc(-c2ccc(N(c3ccc4ccccc4c3)c3cccc(-c4cc5ccccc5c5ccccc45)c3-c3cc4ccccc4c4ccccc34)cc2)cc1]

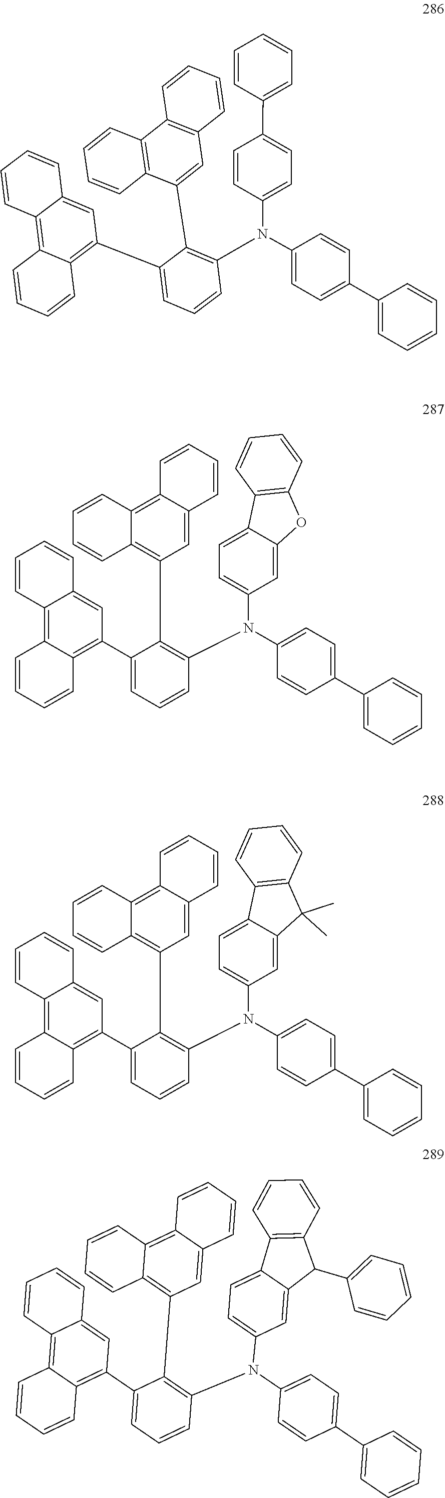 [SMILES: c1ccc(-c2ccc(N(c3ccc(-c4ccccc4)cc3)c3cccc(-c4cc5ccccc5c5ccccc45)c3-c3cc4ccccc4c4ccccc34)cc2)cc1 ; c1ccc(-c2ccc(N(c3ccc4c(c3)oc3ccccc34)c3cccc(-c4cc5ccccc5c5ccccc45)c3-c3cc4ccccc4c4ccccc34)cc2)cc1 ; CC1(C)c2ccccc2-c2ccc(N(c3ccc(-c4ccccc4)cc3)c3cccc(-c4cc5ccccc5c5ccccc45)c3-c3cc4ccccc4c4ccccc34)cc21 ; c1ccc(-c2ccc(N(c3ccc4c(c3)C(c3ccccc3)c3ccccc3-4)c3cccc(-c4cc5ccccc5c5ccccc45)c3-c3cc4ccccc4c4ccccc34)cc2)cc1]

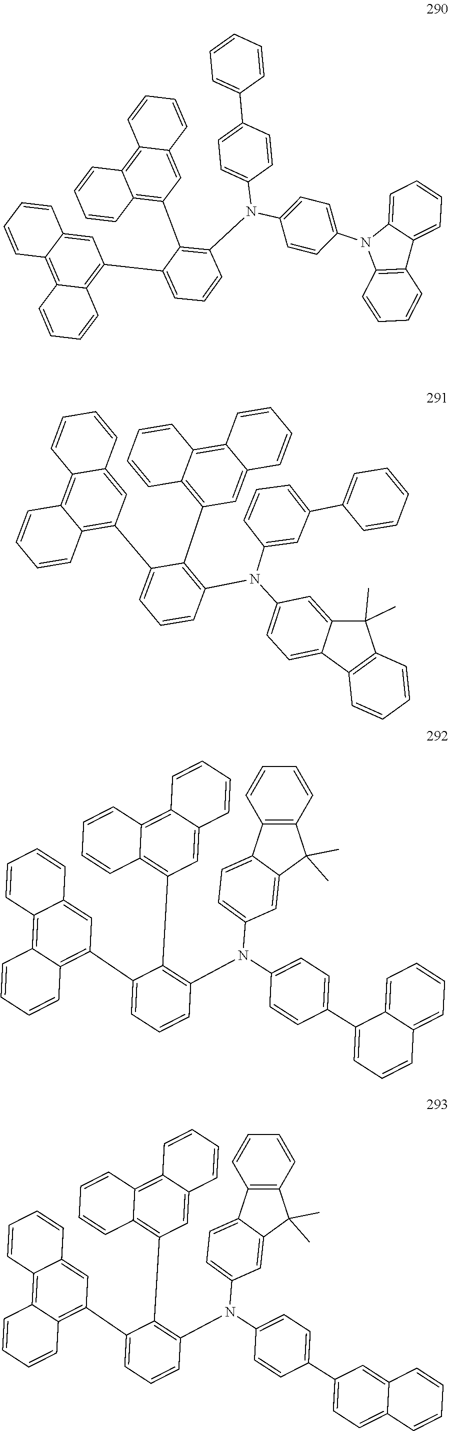 [SMILES: c1ccc(-c2ccc(N(c3ccc(-n4c5ccccc5c5ccccc54)cc3)c3cccc(-c4cc5ccccc5c5ccccc45)c3-c3cc4ccccc4c4ccccc34)cc2)cc1 ; CC1(C)c2ccccc2-c2ccc(N(c3cccc(-c4ccccc4)c3)c3cccc(-c4cc5ccccc5c5ccccc45)c3-c3cc4ccccc4c4ccccc34)cc21 ; CC1(C)c2ccccc2-c2ccc(N(c3ccc(-c4cccc5ccccc45)cc3)c3cccc(-c4cc5ccccc5c5ccccc45)c3-c3cc4ccccc4c4ccccc34)cc21 ; CC1(C)c2ccccc2-c2ccc(N(c3ccc(-c4ccc5ccccc5c4)cc3)c3cccc(-c4cc5ccccc5c5ccccc45)c3-c3cc4ccccc4c4ccccc34)cc21]

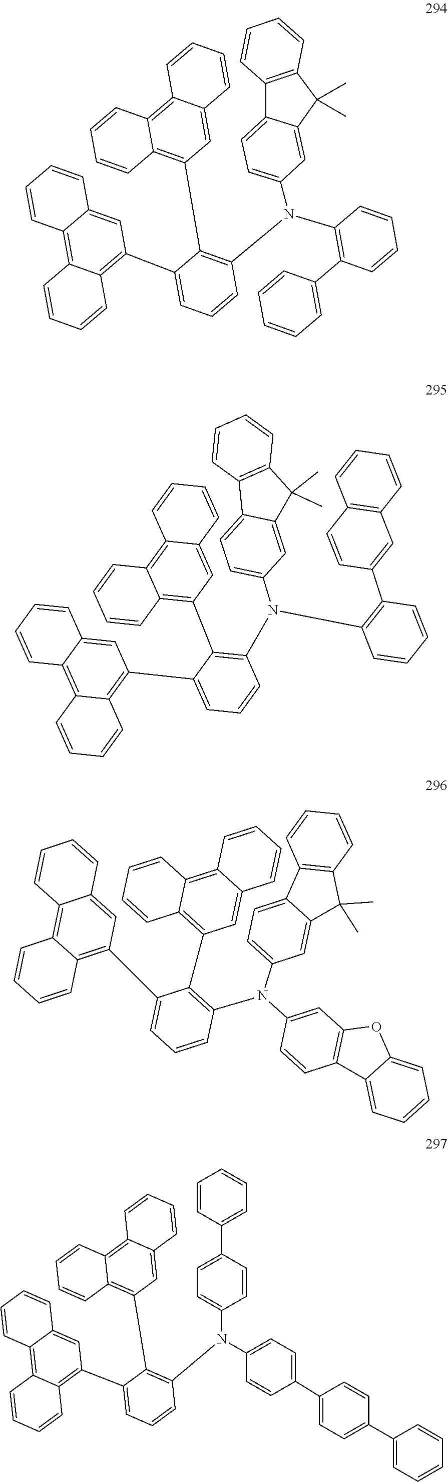 [SMILES: CC1(C)c2ccccc2-c2ccc(N(c3ccccc3-c3ccccc3)c3cccc(-c4cc5ccccc5c5ccccc45)c3-c3cc4ccccc4c4ccccc34)cc21 ; CC1(C)c2ccccc2-c2ccc(N(c3ccccc3-c3ccc4ccccc4c3)c3cccc(-c4cc5ccccc5c5ccccc45)c3-c3cc4ccccc4c4ccccc34)cc21 ; CC1(C)c2ccccc2-c2ccc(N(c3ccc4c(c3)oc3ccccc34)c3cccc(-c4cc5ccccc5c5ccccc45)c3-c3cc4ccccc4c4ccccc34)cc21 ; c1ccc(-c2ccc(-c3ccc(N(c4ccc(-c5ccccc5)cc4)c4cccc(-c5cc6ccccc6c6ccccc56)c4-c4cc5ccccc5c5ccccc45)cc3)cc2)cc1]

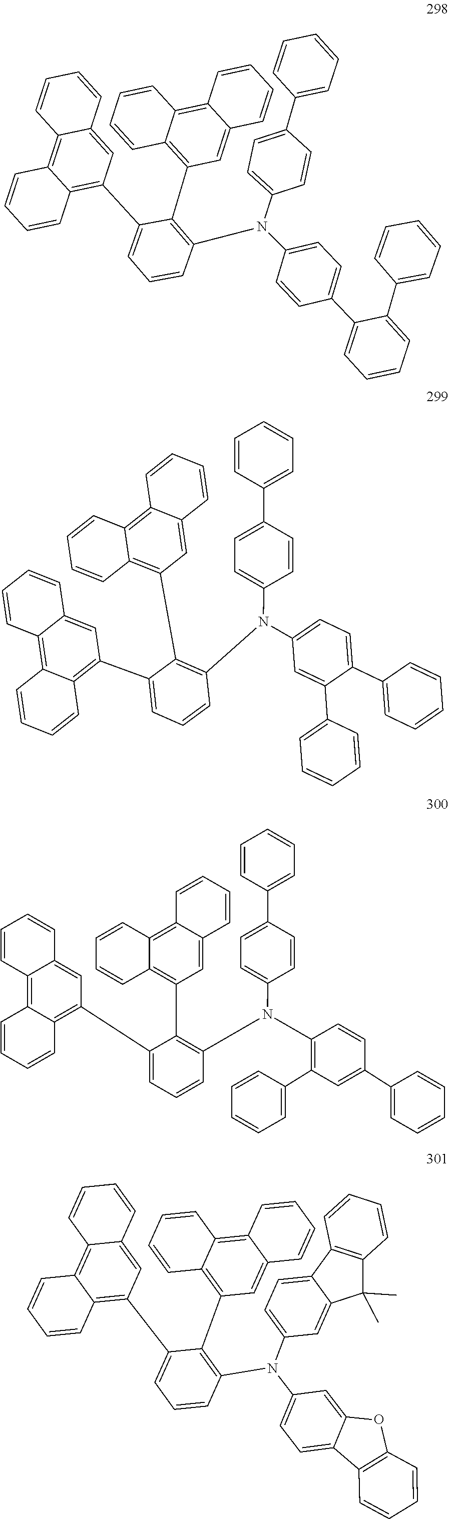 [SMILES: c1ccc(-c2ccc(N(c3ccc(-c4ccccc4-c4ccccc4)cc3)c3cccc(-c4cc5ccccc5c5ccccc45)c3-c3cc4ccccc4c4ccccc34)cc2)cc1 ; c1ccc(-c2ccc(N(c3ccc(-c4ccccc4)c(-c4ccccc4)c3)c3cccc(-c4cc5ccccc5c5ccccc45)c3-c3cc4ccccc4c4ccccc34)cc2)cc1 ; c1ccc(-c2ccc(N(c3ccc(-c4ccccc4)cc3-c3ccccc3)c3cccc(-c4cc5ccccc5c5ccccc45)c3-c3cc4ccccc4c4ccccc34)cc2)cc1 ; CC1(C)c2ccccc2-c2ccc(N(c3ccc4c(c3)oc3ccccc34)c3cccc(-c4cc5ccccc5c5ccccc45)c3-c3cc4ccccc4c4ccccc34)cc21]

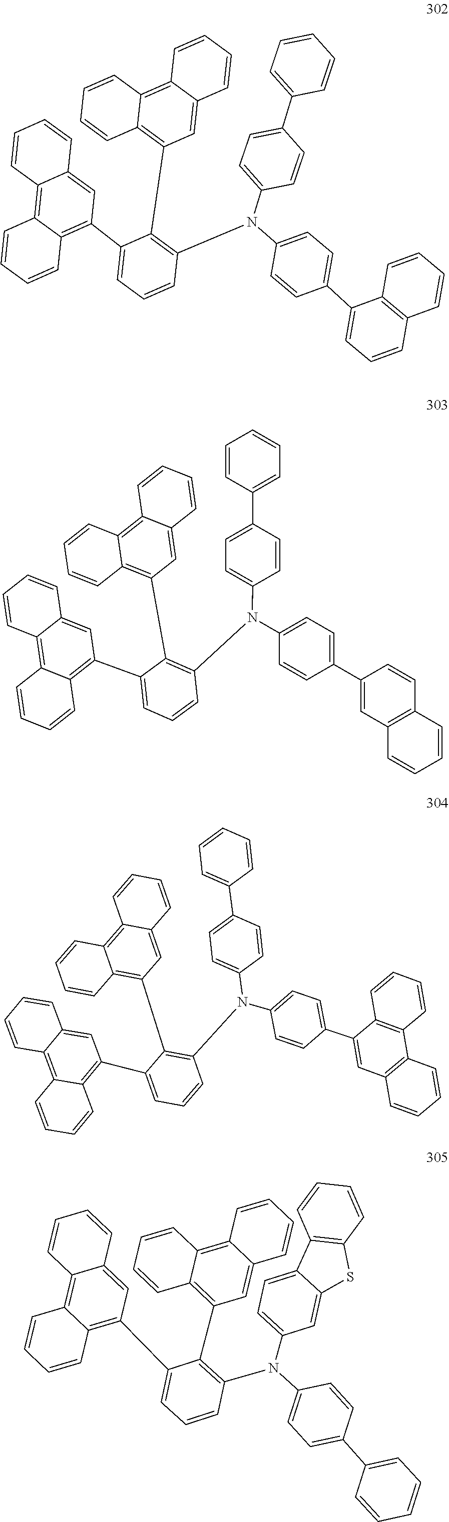 [SMILES: c1ccc(-c2ccc(N(c3ccc(-c4cccc5ccccc45)cc3)c3cccc(-c4cc5ccccc5c5ccccc45)c3-c3cc4ccccc4c4ccccc34)cc2)cc1 ; c1ccc(-c2ccc(N(c3ccc(-c4ccc5ccccc5c4)cc3)c3cccc(-c4cc5ccccc5c5ccccc45)c3-c3cc4ccccc4c4ccccc34)cc2)cc1 ; c1ccc(-c2ccc(N(c3ccc(-c4cc5ccccc5c5ccccc45)cc3)c3cccc(-c4cc5ccccc5c5ccccc45)c3-c3cc4ccccc4c4ccccc34)cc2)cc1 ; c1ccc(-c2ccc(N(c3ccc4c(c3)sc3ccccc34)c3cccc(-c4cc5ccccc5c5ccccc45)c3-c3cc4ccccc4c4ccccc34)cc2)cc1]

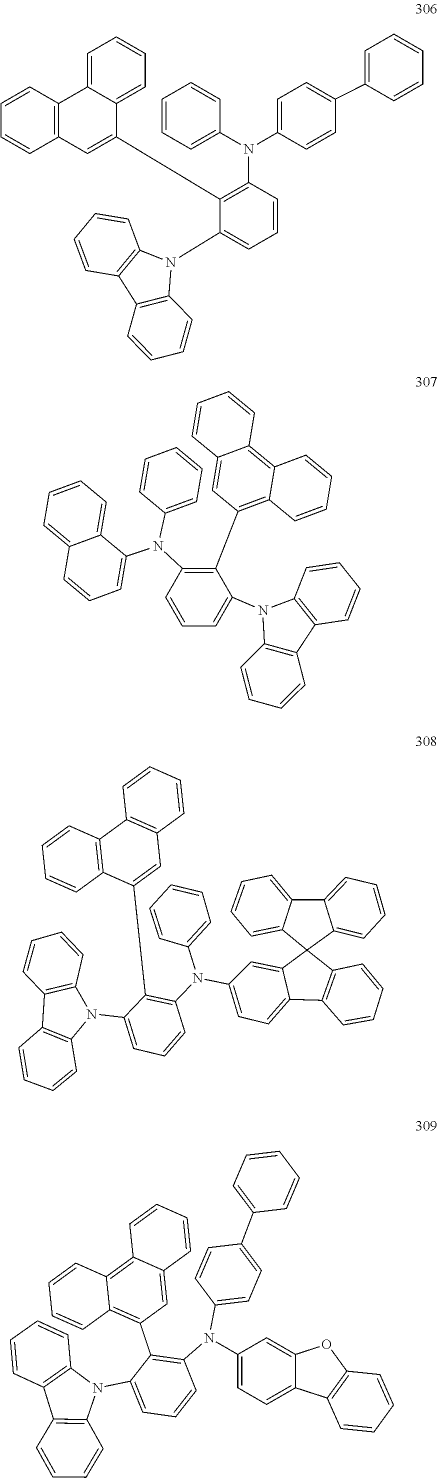 [SMILES: c1ccc(-c2ccc(N(c3ccccc3)c3cccc(-n4c5ccccc5c5ccccc54)c3-c3cc4ccccc4c4ccccc34)cc2)cc1 ; c1ccc(N(c2cccc(-n3c4ccccc4c4ccccc43)c2-c2cc3ccccc3c3ccccc23)c2cccc3ccccc23)cc1 ; c1ccc(N(c2ccc3c(c2)C2(c4ccccc4-c4ccccc42)c2ccccc2-3)c2cccc(-n3c4ccccc4c4ccccc43)c2-c2cc3ccccc3c3ccccc23)cc1 ; c1ccc(-c2ccc(N(c3ccc4c(c3)oc3ccccc34)c3cccc(-n4c5ccccc5c5ccccc54)c3-c3cc4ccccc4c4ccccc34)cc2)cc1]

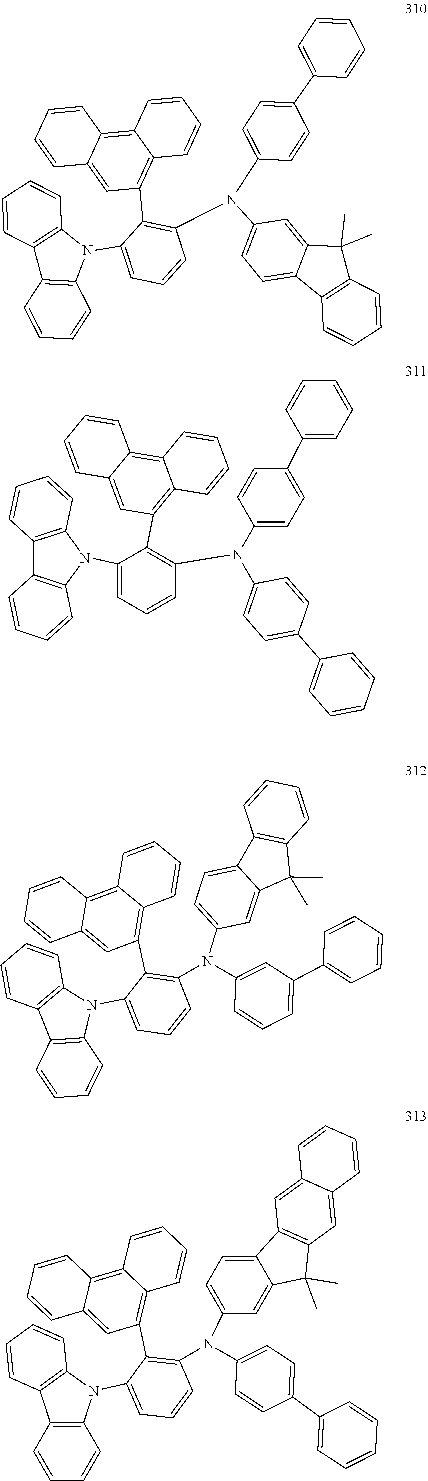

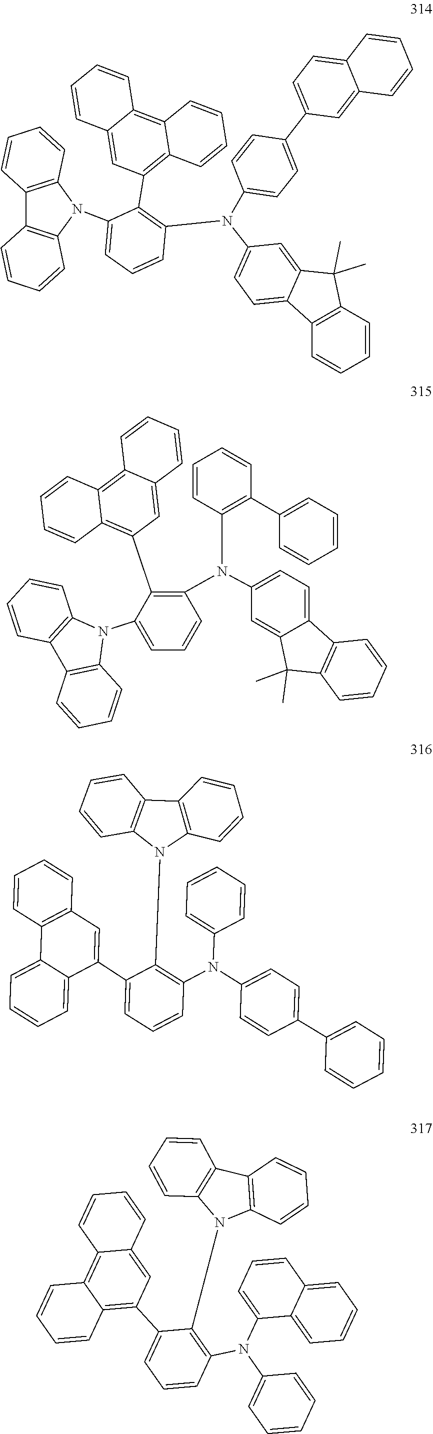 [SMILES: CC1(C)c2ccccc2-c2ccc(N(c3ccc(-c4ccc5ccccc5c4)cc3)c3cccc(-n4c5ccccc5c5ccccc54)c3-c3cc4ccccc4c4ccccc34)cc21 ; CC1(C)c2ccccc2-c2ccc(N(c3ccccc3-c3ccccc3)c3cccc(-n4c5ccccc5c5ccccc54)c3-c3cc4ccccc4c4ccccc34)cc21 ; c1ccc(-c2ccc(N(c3ccccc3)c3cccc(-c4cc5ccccc5c5ccccc45)c3-n3c4ccccc4c4ccccc43)cc2)cc1 ; c1ccc(N(c2cccc(-c3cc4ccccc4c4ccccc34)c2-n2c3ccccc3c3ccccc32)c2cccc3ccccc23)cc1]

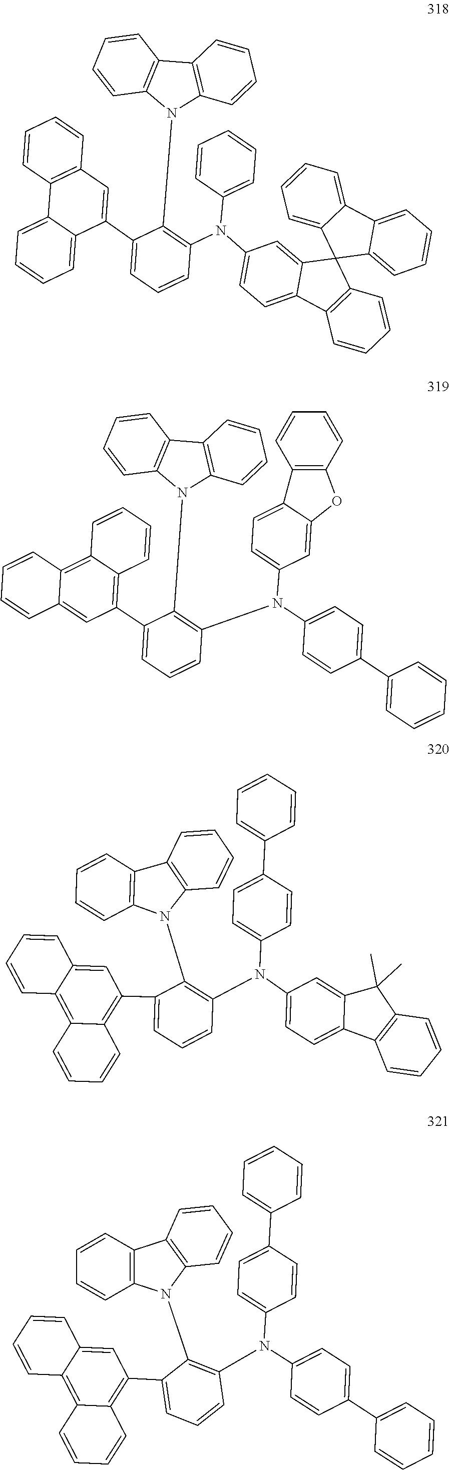 [SMILES: c1ccc(N(c2ccc3c(c2)C2(c4ccccc4-c4ccccc42)c2ccccc2-3)c2cccc(-c3cc4ccccc4c4ccccc34)c2-n2c3ccccc3c3ccccc32)cc1 ; c1ccc(-c2ccc(N(c3ccc4c(c3)oc3ccccc34)c3cccc(-c4cc5ccccc5c5ccccc45)c3-n3c4ccccc4c4ccccc43)cc2)cc1 ; CC1(C)c2ccccc2-c2ccc(N(c3ccc(-c4ccccc4)cc3)c3cccc(-c4cc5ccccc5c5ccccc45)c3-n3c4ccccc4c4ccccc43)cc21 ; c1ccc(-c2ccc(N(c3ccc(-c4ccccc4)cc3)c3cccc(-c4cc5ccccc5c5ccccc45)c3-n3c4ccccc4c4ccccc43)cc2)cc1]

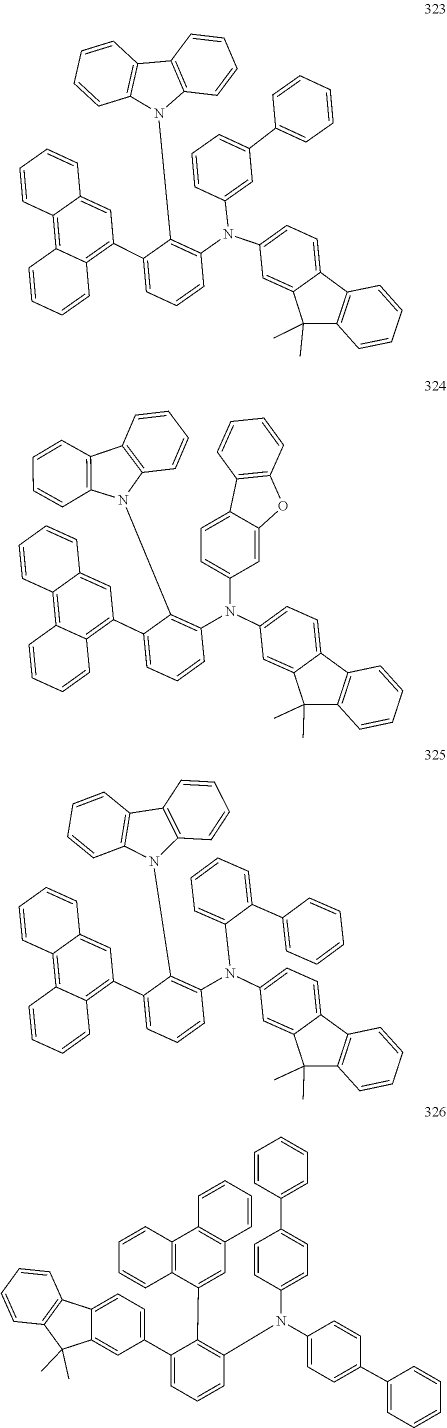

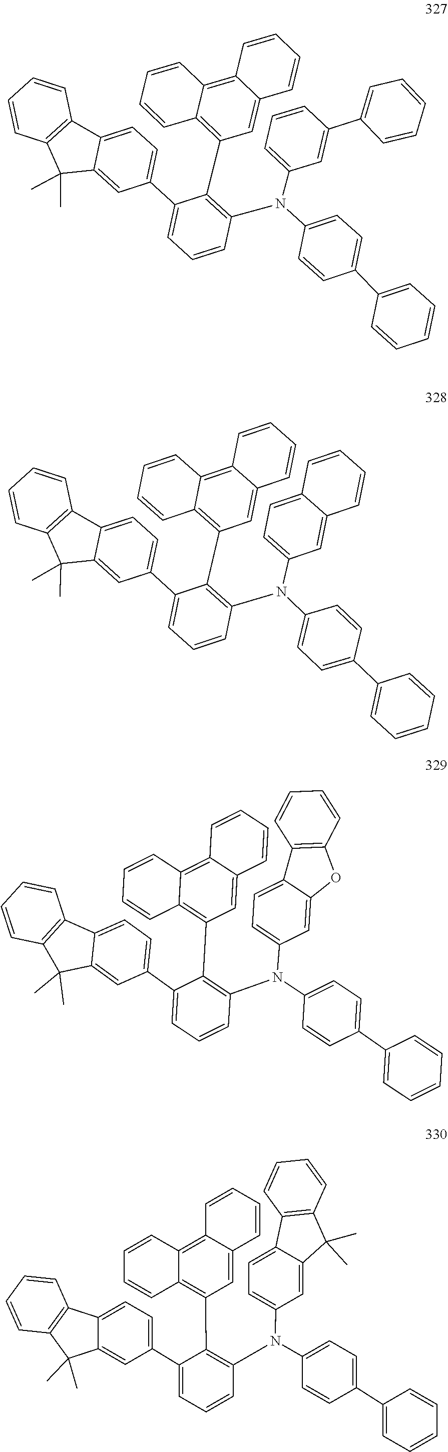 [SMILES: CC1(C)c2ccccc2-c2ccc(-c3cccc(N(c4ccc(-c5ccccc5)cc4)c4cccc(-c5ccccc5)c4)c3-c3cc4ccccc4c4ccccc34)cc21 ; CC1(C)c2ccccc2-c2ccc(-c3cccc(N(c4ccc(-c5ccccc5)cc4)c4ccc5ccccc5c4)c3-c3cc4ccccc4c4ccccc34)cc21 ; CC1(C)c2ccccc2-c2ccc(-c3cccc(N(c4ccc(-c5ccccc5)cc4)c4ccc5c(c4)oc4ccccc45)c3-c3cc4ccccc4c4ccccc34)cc21 ; CC1(C)c2ccccc2-c2ccc(-c3cccc(N(c4ccc(-c5ccccc5)cc4)c4ccc5c(c4)C(C)(C)c4ccccc4-5)c3-c3cc4ccccc4c4ccccc34)cc21]

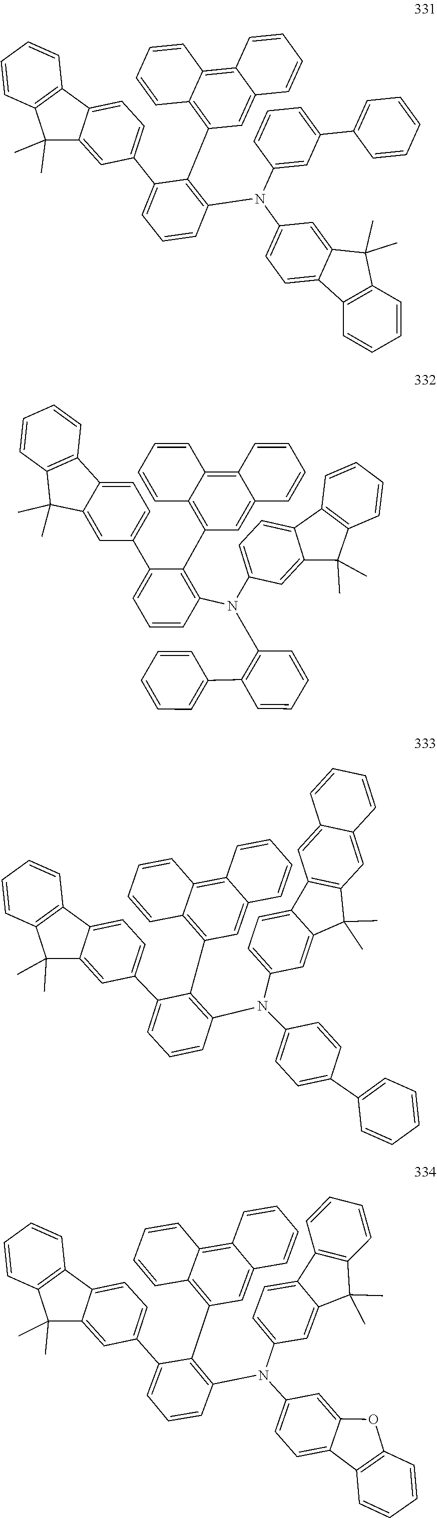 [SMILES: CC1(C)c2ccccc2-c2ccc(-c3cccc(N(c4cccc(-c5ccccc5)c4)c4ccc5c(c4)C(C)(C)c4ccccc4-5)c3-c3cc4ccccc4c4ccccc34)cc21 ; CC1(C)c2ccccc2-c2ccc(-c3cccc(N(c4ccc5c(c4)C(C)(C)c4ccccc4-5)c4ccccc4-c4ccccc4)c3-c3cc4ccccc4c4ccccc34)cc21 ; CC1(C)c2ccccc2-c2ccc(-c3cccc(N(c4ccc(-c5ccccc5)cc4)c4ccc5c(c4)C(C)(C)c4cc6ccccc6cc4-5)c3-c3cc4ccccc4c4ccccc34)cc21 ; CC1(C)c2ccccc2-c2ccc(-c3cccc(N(c4ccc5c(c4)C(C)(C)c4ccccc4-5)c4ccc5c(c4)oc4ccccc45)c3-c3cc4ccccc4c4ccccc34)cc21]

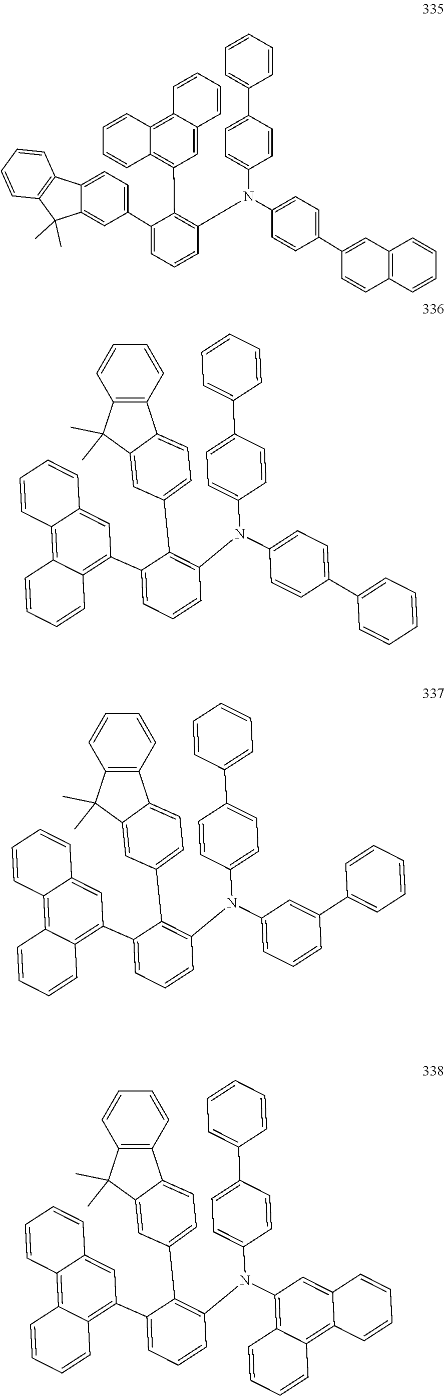 [SMILES: CC1(C)c2ccccc2-c2ccc(-c3cccc(N(c4ccc(-c5ccccc5)cc4)c4ccc(-c5ccc6ccccc6c5)cc4)c3-c3cc4ccccc4c4ccccc34)cc21 ; CC1(C)c2ccccc2-c2ccc(-c3c(-c4cc5ccccc5c5ccccc45)cccc3N(c3ccc(-c4ccccc4)cc3)c3ccc(-c4ccccc4)cc3)cc21 ; CC1(C)c2ccccc2-c2ccc(-c3c(-c4cc5ccccc5c5ccccc45)cccc3N(c3ccc(-c4ccccc4)cc3)c3cccc(-c4ccccc4)c3)cc21 ; CC1(C)c2ccccc2-c2ccc(-c3c(-c4cc5ccccc5c5ccccc45)cccc3N(c3ccc(-c4ccccc4)cc3)c3cc4ccccc4c4ccccc34)cc21]

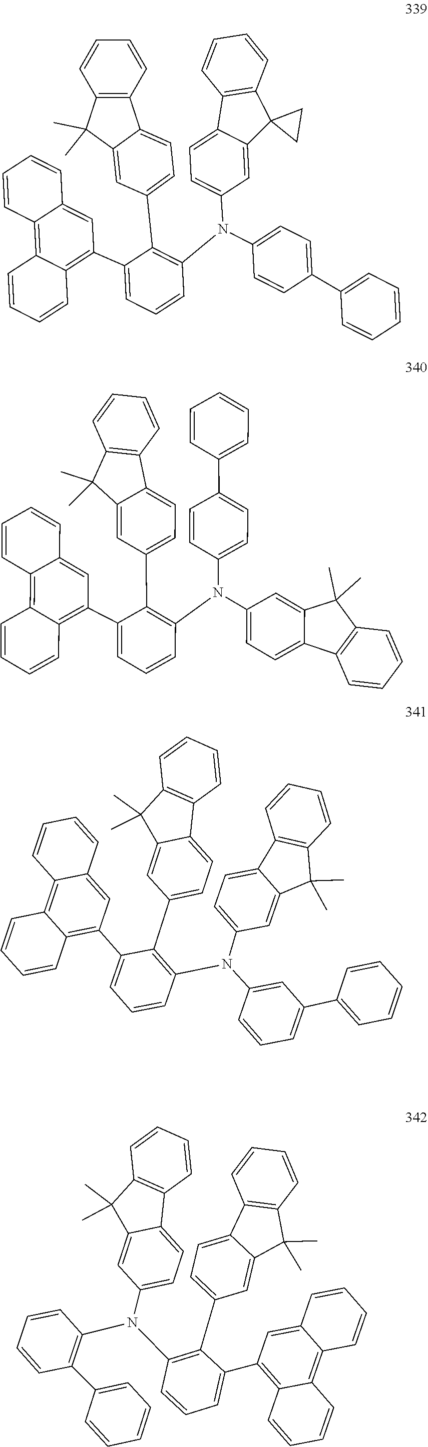 [SMILES: CC1(C)c2ccccc2-c2ccc(-c3c(-c4cc5ccccc5c5ccccc45)cccc3N(c3ccc(-c4ccccc4)cc3)c3ccc4c(c3)C3(CC3)c3ccccc3-4)cc21 ; CC1(C)c2ccccc2-c2ccc(-c3c(-c4cc5ccccc5c5ccccc45)cccc3N(c3ccc(-c4ccccc4)cc3)c3ccc4c(c3)C(C)(C)c3ccccc3-4)cc21 ; CC1(C)c2ccccc2-c2ccc(-c3c(-c4cc5ccccc5c5ccccc45)cccc3N(c3cccc(-c4ccccc4)c3)c3ccc4c(c3)C(C)(C)c3ccccc3-4)cc21 ; CC1(C)c2ccccc2-c2ccc(-c3c(-c4cc5ccccc5c5ccccc45)cccc3N(c3ccc4c(c3)C(C)(C)c3ccccc3-4)c3ccccc3-c3ccccc3)cc21]

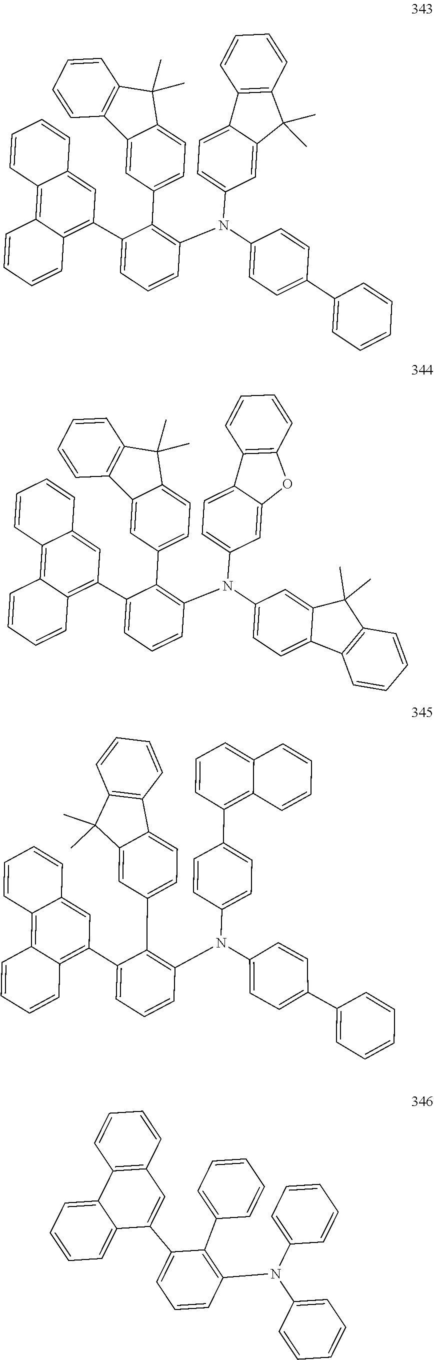 [SMILES: CC1(C)c2ccccc2-c2cc(-c3c(-c4cc5ccccc5c5ccccc45)cccc3N(c3ccc(-c4ccccc4)cc3)c3ccc4c(c3)C(C)(C)c3ccccc3-4)ccc21 ; CC1(C)c2ccccc2-c2cc(-c3c(-c4cc5ccccc5c5ccccc45)cccc3N(c3ccc4c(c3)C(C)(C)c3ccccc3-4)c3ccc4c(c3)oc3ccccc34)ccc21 ; CC1(C)c2ccccc2-c2ccc(-c3c(-c4cc5ccccc5c5ccccc45)cccc3N(c3ccc(-c4ccccc4)cc3)c3ccc(-c4cccc5ccccc45)cc3)cc21 ; c1ccc(-c2c(-c3cc4ccccc4c4ccccc34)cccc2N(c2ccccc2)c2ccccc2)cc1]

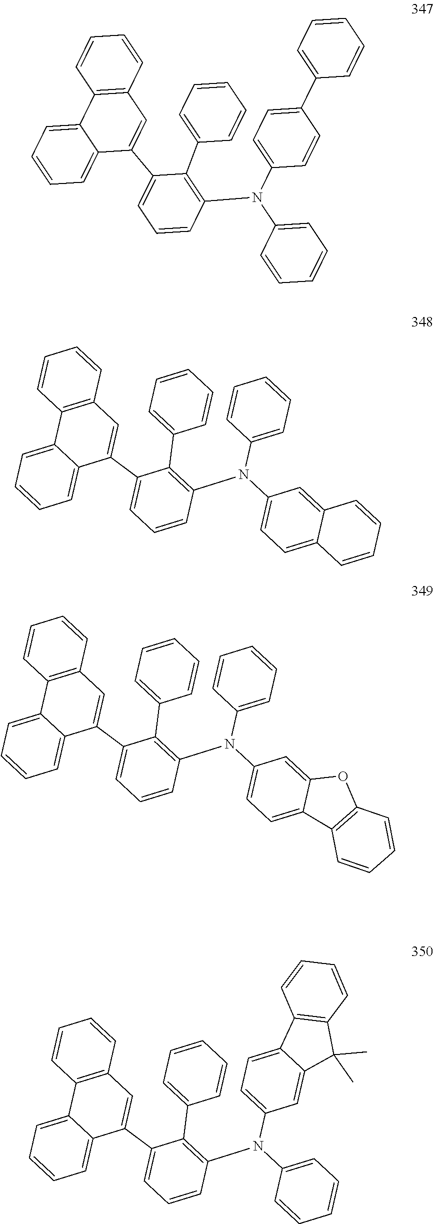 [SMILES: c1ccc(-c2ccc(N(c3ccccc3)c3cccc(-c4cc5ccccc5c5ccccc45)c3-c3ccccc3)cc2)cc1 ; c1ccc(-c2c(-c3cc4ccccc4c4ccccc34)cccc2N(c2ccccc2)c2ccc3ccccc3c2)cc1 ; c1ccc(-c2c(-c3cc4ccccc4c4ccccc34)cccc2N(c2ccccc2)c2ccc3c(c2)oc2ccccc23)cc1 ; CC1(C)c2ccccc2-c2ccc(N(c3ccccc3)c3cccc(-c4cc5ccccc5c5ccccc45)c3-c3ccccc3)cc21]

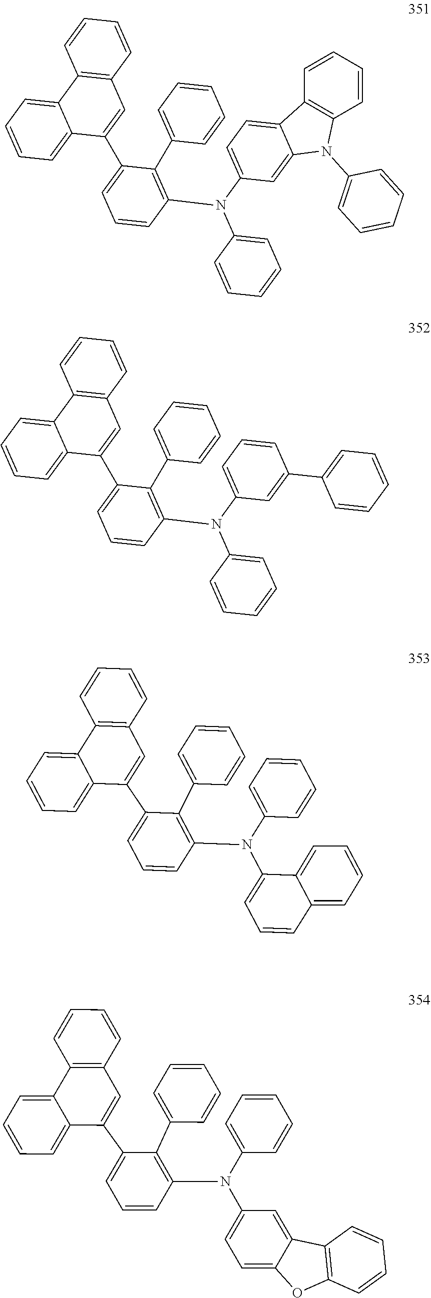 [SMILES: c1ccc(-c2c(-c3cc4ccccc4c4ccccc34)cccc2N(c2ccccc2)c2ccc3c4ccccc4n(-c4ccccc4)c3c2)cc1 ; c1ccc(-c2cccc(N(c3ccccc3)c3cccc(-c4cc5ccccc5c5ccccc45)c3-c3ccccc3)c2)cc1 ; c1ccc(-c2c(-c3cc4ccccc4c4ccccc34)cccc2N(c2ccccc2)c2cccc3ccccc23)cc1 ; c1ccc(-c2c(-c3cc4ccccc4c4ccccc34)cccc2N(c2ccccc2)c2ccc3oc4ccccc4c3c2)cc1]

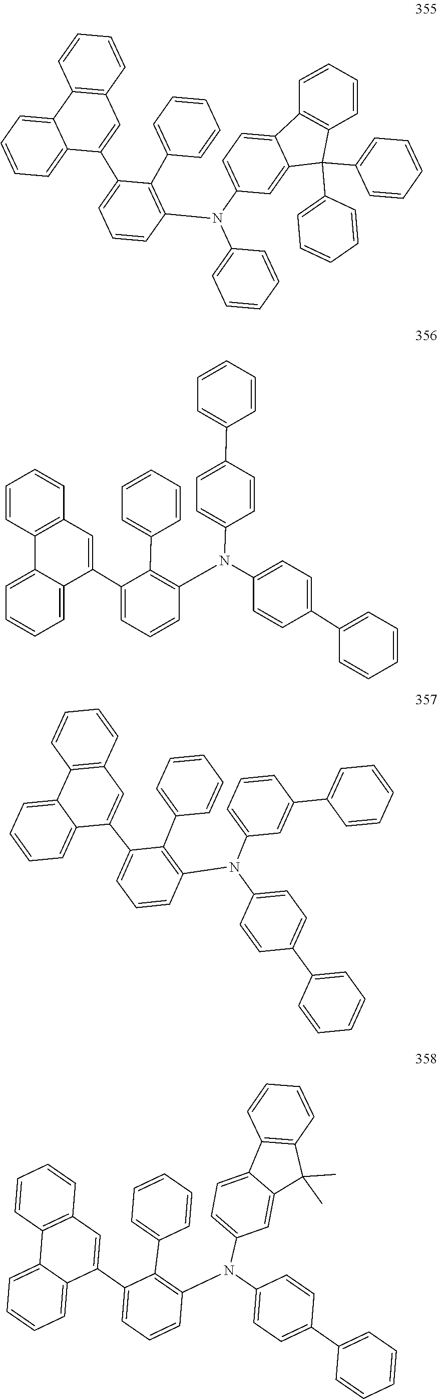 [SMILES: c1ccc(-c2c(-c3cc4ccccc4c4ccccc34)cccc2N(c2ccccc2)c2ccc3c(c2)C(c2ccccc2)(c2ccccc2)c2ccccc2-3)cc1 ; c1ccc(-c2ccc(N(c3ccc(-c4ccccc4)cc3)c3cccc(-c4cc5ccccc5c5ccccc45)c3-c3ccccc3)cc2)cc1 ; c1ccc(-c2ccc(N(c3cccc(-c4ccccc4)c3)c3cccc(-c4cc5ccccc5c5ccccc45)c3-c3ccccc3)cc2)cc1 ; CC1(C)c2ccccc2-c2ccc(N(c3ccc(-c4ccccc4)cc3)c3cccc(-c4cc5ccccc5c5ccccc45)c3-c3ccccc3)cc21]

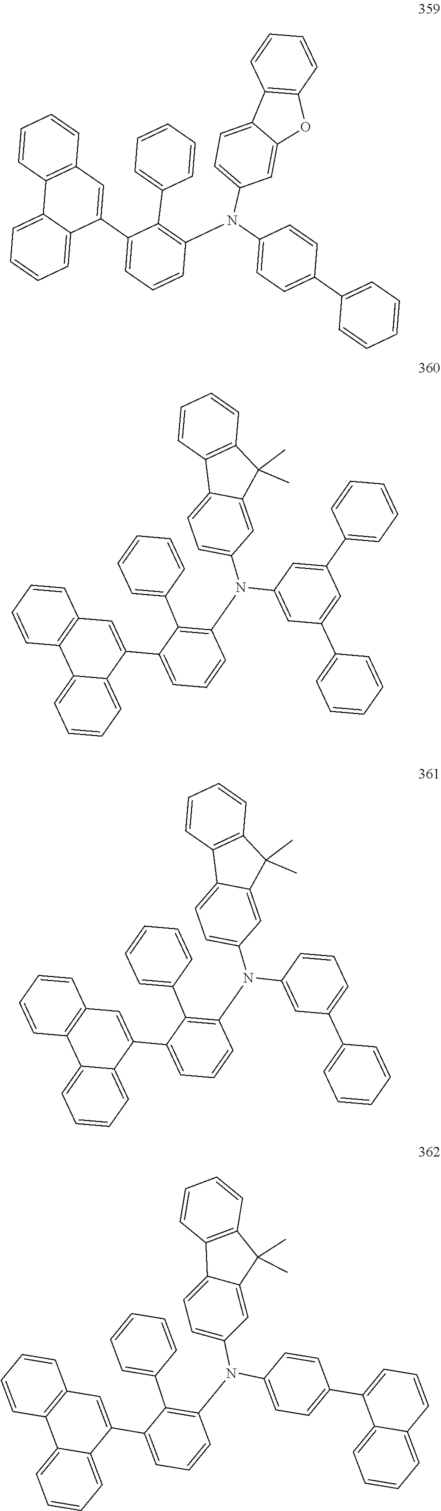 [SMILES: c1ccc(-c2ccc(N(c3ccc4c(c3)oc3ccccc34)c3cccc(-c4cc5ccccc5c5ccccc45)c3-c3ccccc3)cc2)cc1 ; CC1(C)c2ccccc2-c2ccc(N(c3cc(-c4ccccc4)cc(-c4ccccc4)c3)c3cccc(-c4cc5ccccc5c5ccccc45)c3-c3ccccc3)cc21 ; CC1(C)c2ccccc2-c2ccc(N(c3cccc(-c4ccccc4)c3)c3cccc(-c4cc5ccccc5c5ccccc45)c3-c3ccccc3)cc21 ; CC1(C)c2ccccc2-c2ccc(N(c3ccc(-c4cccc5ccccc45)cc3)c3cccc(-c4cc5ccccc5c5ccccc45)c3-c3ccccc3)cc21]

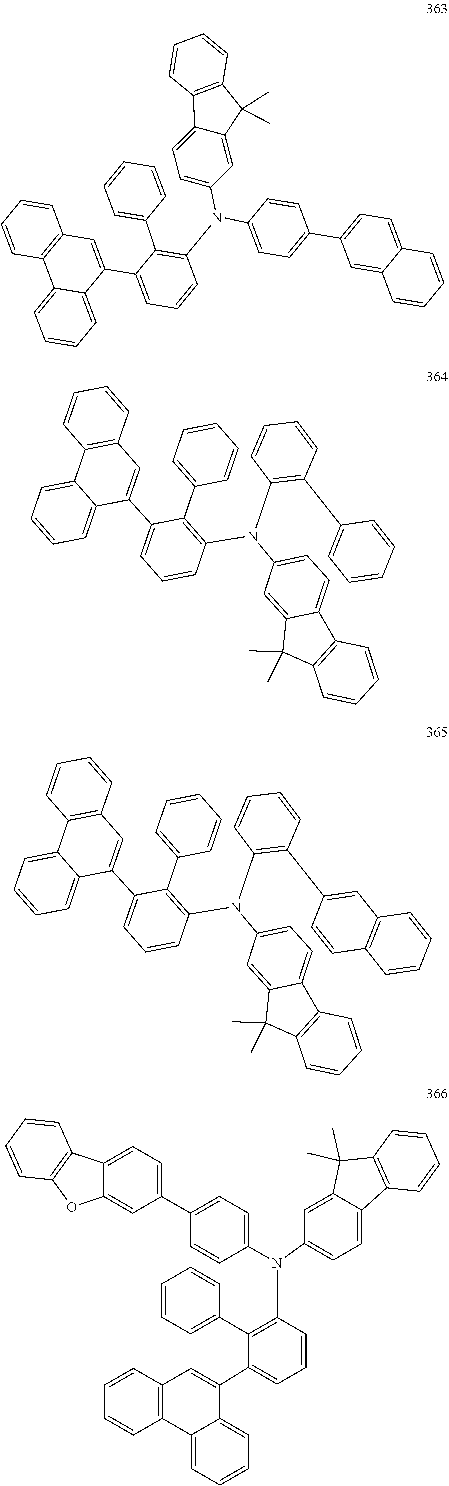

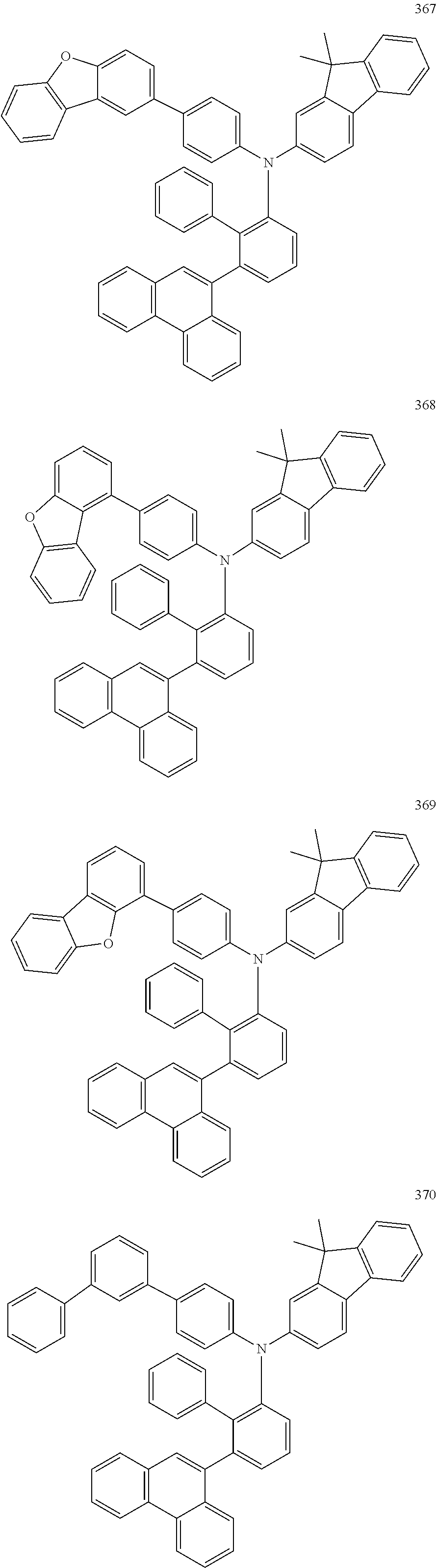 [SMILES: CC1(C)c2ccccc2-c2ccc(N(c3ccc(-c4ccc5oc6ccccc6c5c4)cc3)c3cccc(-c4cc5ccccc5c5ccccc45)c3-c3ccccc3)cc21 ; CC1(C)c2ccccc2-c2ccc(N(c3ccc(-c4cccc5oc6ccccc6c45)cc3)c3cccc(-c4cc5ccccc5c5ccccc45)c3-c3ccccc3)cc21 ; CC1(C)c2ccccc2-c2ccc(N(c3ccc(-c4cccc5c4oc4ccccc45)cc3)c3cccc(-c4cc5ccccc5c5ccccc45)c3-c3ccccc3)cc21 ; CC1(C)c2ccccc2-c2ccc(N(c3ccc(-c4cccc(-c5ccccc5)c4)cc3)c3cccc(-c4cc5ccccc5c5ccccc45)c3-c3ccccc3)cc21]

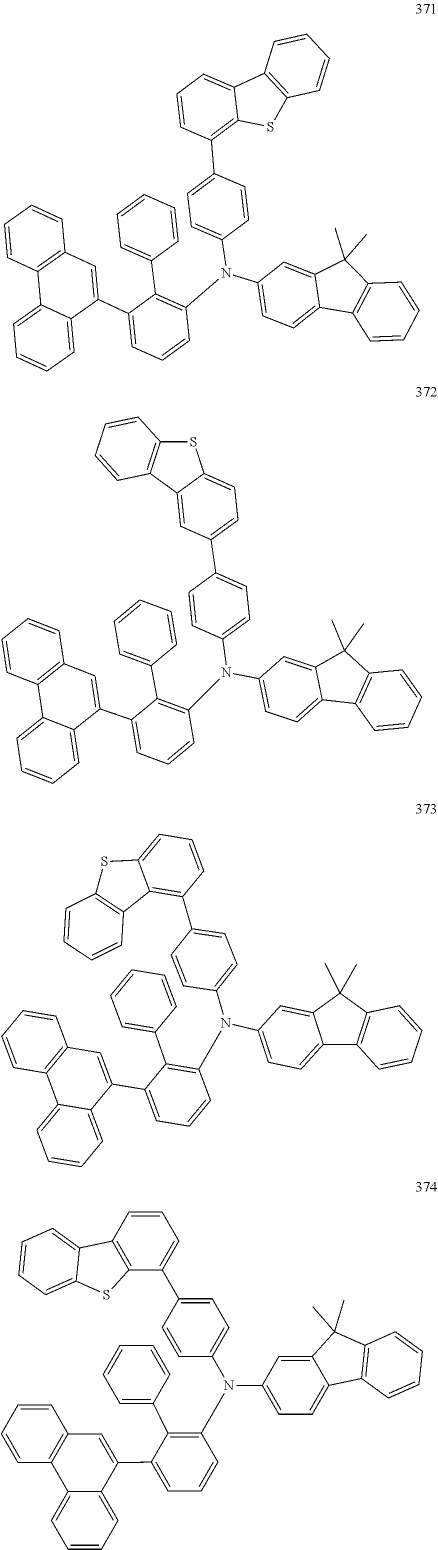 [SMILES: CC1(C)c2ccccc2-c2ccc(N(c3ccc(-c4cccc5c4sc4ccccc45)cc3)c3cccc(-c4cc5ccccc5c5ccccc45)c3-c3ccccc3)cc21 ; CC1(C)c2ccccc2-c2ccc(N(c3ccc(-c4ccc5sc6ccccc6c5c4)cc3)c3cccc(-c4cc5ccccc5c5ccccc45)c3-c3ccccc3)cc21 ; CC1(C)c2ccccc2-c2ccc(N(c3ccc(-c4cccc5sc6ccccc6c45)cc3)c3cccc(-c4cc5ccccc5c5ccccc45)c3-c3ccccc3)cc21 ; CC1(C)c2ccccc2-c2ccc(N(c3ccc(-c4cccc5c4sc4ccccc45)cc3)c3cccc(-c4cc5ccccc5c5ccccc45)c3-c3ccccc3)cc21]

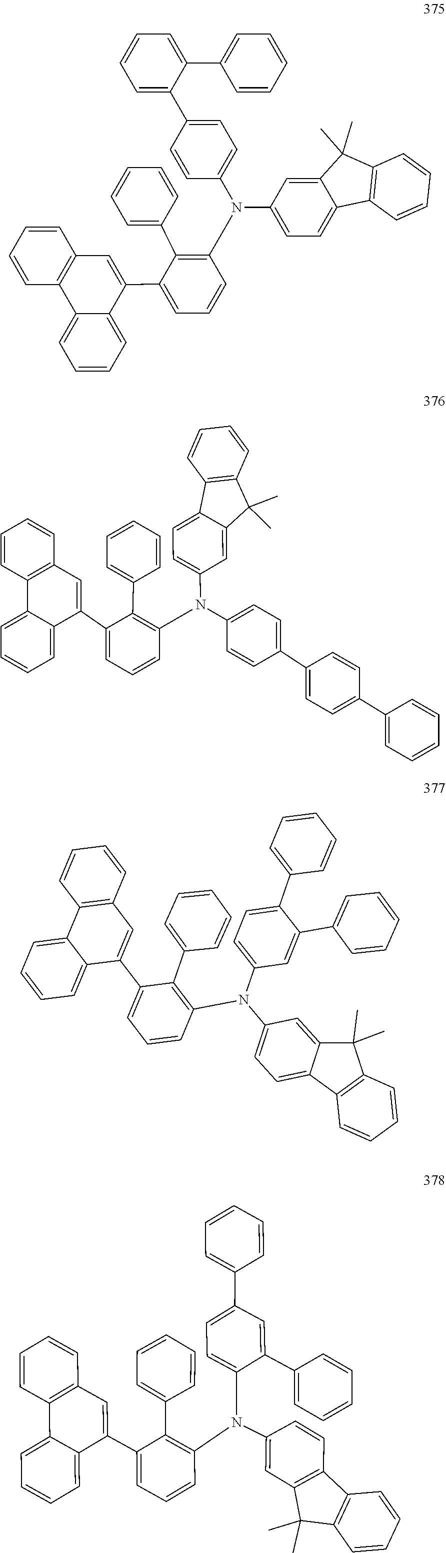 [SMILES: CC1(C)c2ccccc2-c2ccc(N(c3ccc(-c4ccccc4-c4ccccc4)cc3)c3cccc(-c4cc5ccccc5c5ccccc45)c3-c3ccccc3)cc21 ; CC1(C)c2ccccc2-c2ccc(N(c3ccc(-c4ccc(-c5ccccc5)cc4)cc3)c3cccc(-c4cc5ccccc5c5ccccc45)c3-c3ccccc3)cc21 ; CC1(C)c2ccccc2-c2ccc(N(c3ccc(-c4ccccc4)c(-c4ccccc4)c3)c3cccc(-c4cc5ccccc5c5ccccc45)c3-c3ccccc3)cc21 ; CC1(C)c2ccccc2-c2ccc(N(c3ccc(-c4ccccc4)cc3-c3ccccc3)c3cccc(-c4cc5ccccc5c5ccccc45)c3-c3ccccc3)cc21]

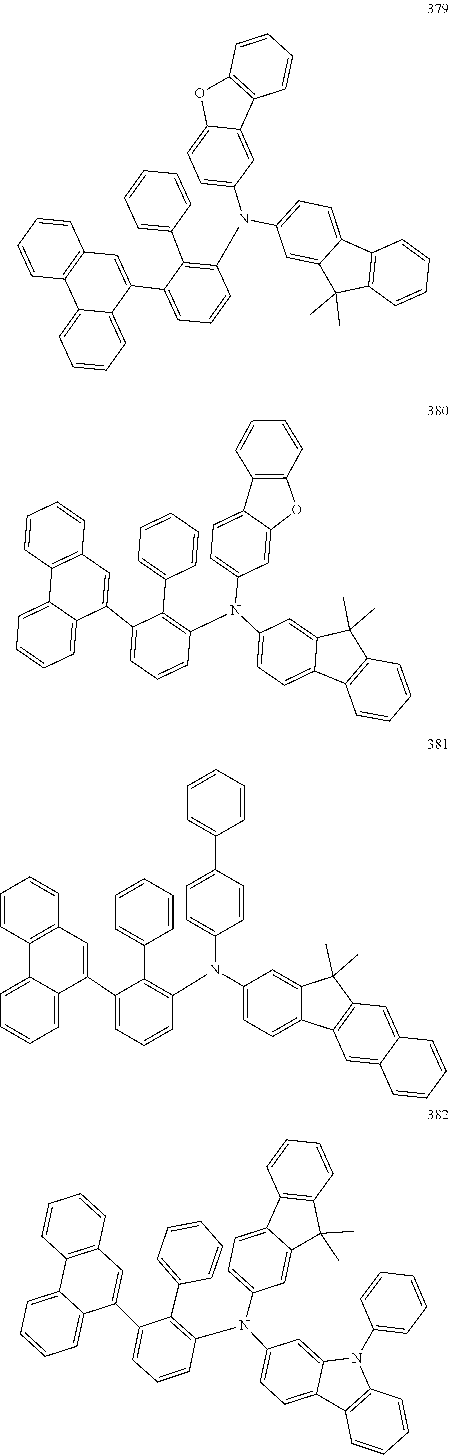 [SMILES: CC1(C)c2ccccc2-c2ccc(N(c3ccc4oc5ccccc5c4c3)c3cccc(-c4cc5ccccc5c5ccccc45)c3-c3ccccc3)cc21 ; CC1(C)c2ccccc2-c2ccc(N(c3ccc4c(c3)oc3ccccc34)c3cccc(-c4cc5ccccc5c5ccccc45)c3-c3ccccc3)cc21 ; CC1(C)c2cc(N(c3ccc(-c4ccccc4)cc3)c3cccc(-c4cc5ccccc5c5ccccc45)c3-c3ccccc3)ccc2-c2cc3ccccc3cc21 ; CC1(C)c2ccccc2-c2ccc(N(c3ccc4c5ccccc5n(-c5ccccc5)c4c3)c3cccc(-c4cc5ccccc5c5ccccc45)c3-c3ccccc3)cc21]

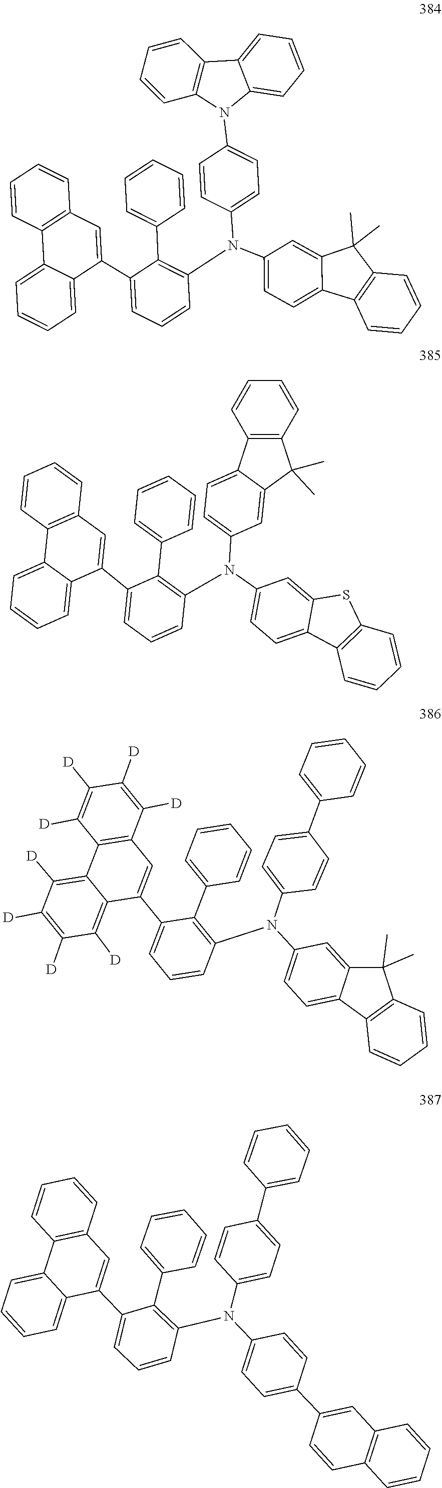 [SMILES: CC1(C)c2ccccc2-c2ccc(N(c3ccc(-n4c5ccccc5c5ccccc54)cc3)c3cccc(-c4cc5ccccc5c5ccccc45)c3-c3ccccc3)cc21 ; CC1(C)c2ccccc2-c2ccc(N(c3ccc4c(c3)sc3ccccc34)c3cccc(-c4cc5ccccc5c5ccccc45)c3-c3ccccc3)cc21 ; [2H]c1c([2H])c([2H])c2c(cc(-c3cccc(N(c4ccc(-c5ccccc5)cc4)c4ccc5c(c4)C(C)(C)c4ccccc4-5)c3-c3ccccc3)c3c([2H])c([2H])c([2H])c([2H])c32)c1[2H] ; c1ccc(-c2ccc(N(c3ccc(-c4ccc5ccccc5c4)cc3)c3cccc(-c4cc5ccccc5c5ccccc45)c3-c3ccccc3)cc2)cc1]

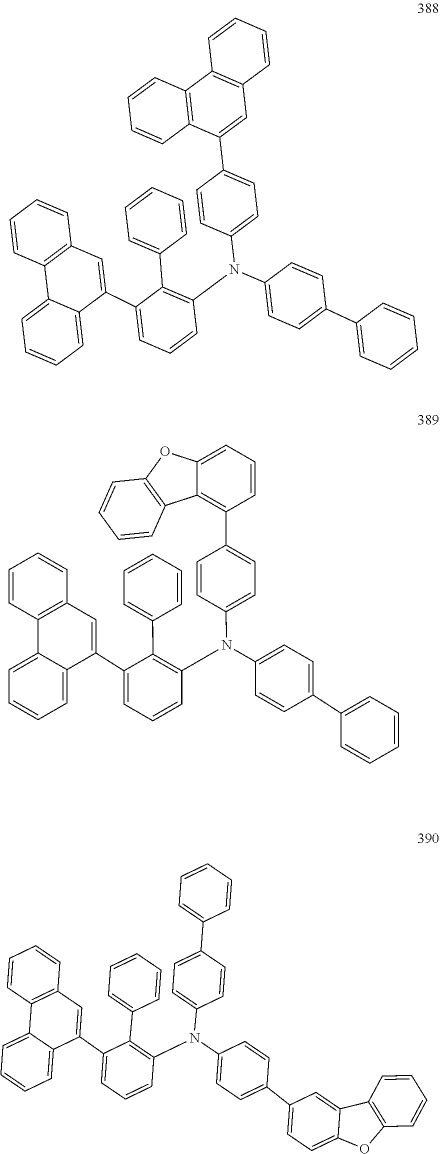 [SMILES: c1ccc(-c2ccc(N(c3ccc(-c4cc5ccccc5c5ccccc45)cc3)c3cccc(-c4cc5ccccc5c5ccccc45)c3-c3ccccc3)cc2)cc1 ; c1ccc(-c2ccc(N(c3ccc(-c4cccc5oc6ccccc6c45)cc3)c3cccc(-c4cc5ccccc5c5ccccc45)c3-c3ccccc3)cc2)cc1 ; c1ccc(-c2ccc(N(c3ccc(-c4ccc5oc6ccccc6c5c4)cc3)c3cccc(-c4cc5ccccc5c5ccccc45)c3-c3ccccc3)cc2)cc1]

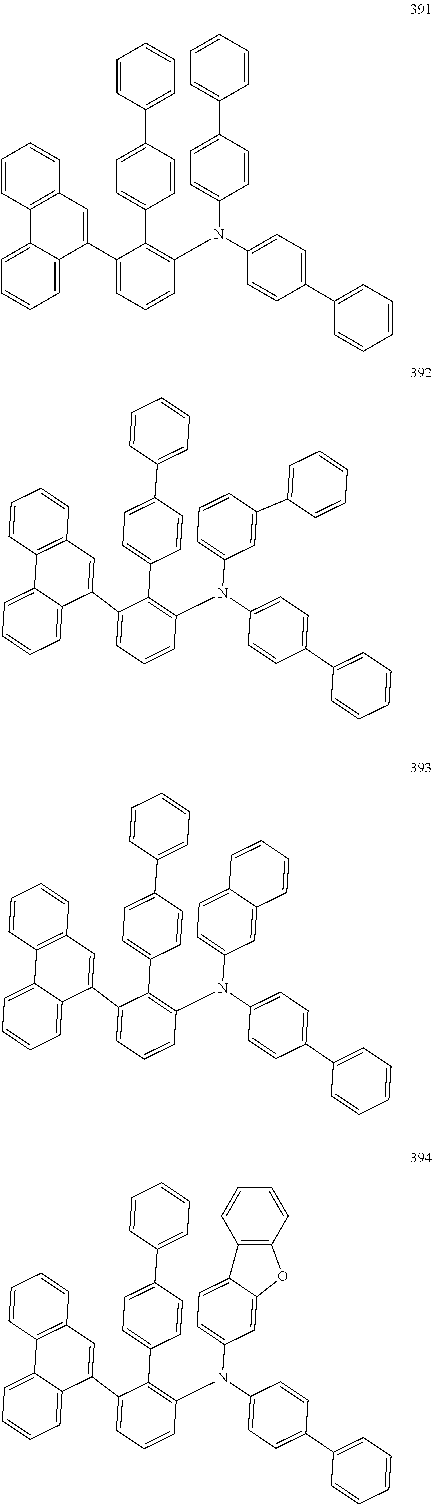 [SMILES: c1ccc(-c2ccc(-c3c(-c4cc5ccccc5c5ccccc45)cccc3N(c3ccc(-c4ccccc4)cc3)c3ccc(-c4ccccc4)cc3)cc2)cc1 ; c1ccc(-c2ccc(-c3c(-c4cc5ccccc5c5ccccc45)cccc3N(c3ccc(-c4ccccc4)cc3)c3cccc(-c4ccccc4)c3)cc2)cc1 ; c1ccc(-c2ccc(-c3c(-c4cc5ccccc5c5ccccc45)cccc3N(c3ccc(-c4ccccc4)cc3)c3ccc4ccccc4c3)cc2)cc1 ; c1ccc(-c2ccc(-c3c(-c4cc5ccccc5c5ccccc45)cccc3N(c3ccc(-c4ccccc4)cc3)c3ccc4c(c3)oc3ccccc34)cc2)cc1]

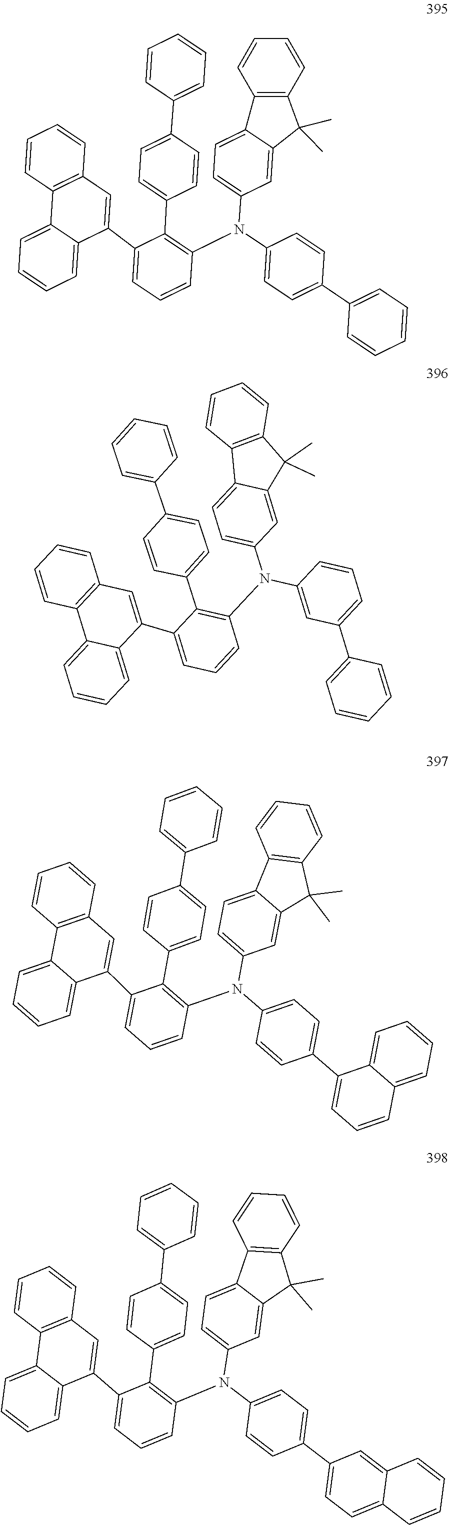 [SMILES: CC1(C)c2ccccc2-c2ccc(N(c3ccc(-c4ccccc4)cc3)c3cccc(-c4cc5ccccc5c5ccccc45)c3-c3ccc(-c4ccccc4)cc3)cc21 ; CC1(C)c2ccccc2-c2ccc(N(c3cccc(-c4ccccc4)c3)c3cccc(-c4cc5ccccc5c5ccccc45)c3-c3ccc(-c4ccccc4)cc3)cc21 ; CC1(C)c2ccccc2-c2ccc(N(c3ccc(-c4cccc5ccccc45)cc3)c3cccc(-c4cc5ccccc5c5ccccc45)c3-c3ccc(-c4ccccc4)cc3)cc21 ; CC1(C)c2ccccc2-c2ccc(N(c3ccc(-c4ccc5ccccc5c4)cc3)c3cccc(-c4cc5ccccc5c5ccccc45)c3-c3ccc(-c4ccccc4)cc3)cc21]

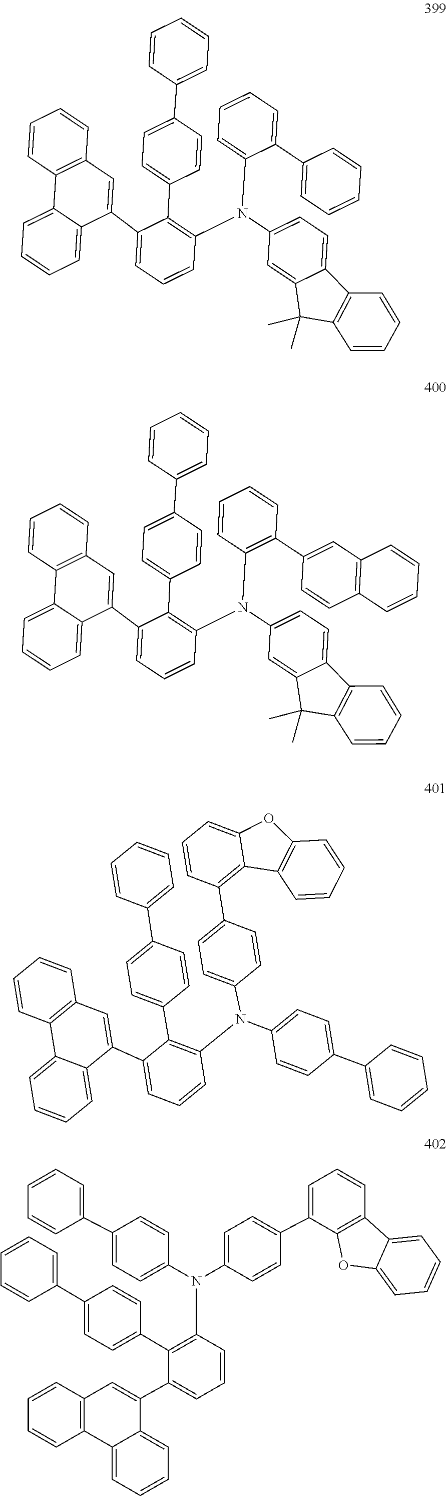 [SMILES: CC1(C)c2ccccc2-c2ccc(N(c3ccccc3-c3ccccc3)c3cccc(-c4cc5ccccc5c5ccccc45)c3-c3ccc(-c4ccccc4)cc3)cc21 ; CC1(C)c2ccccc2-c2ccc(N(c3ccccc3-c3ccc4ccccc4c3)c3cccc(-c4cc5ccccc5c5ccccc45)c3-c3ccc(-c4ccccc4)cc3)cc21 ; c1ccc(-c2ccc(-c3c(-c4cc5ccccc5c5ccccc45)cccc3N(c3ccc(-c4ccccc4)cc3)c3ccc(-c4cccc5oc6ccccc6c45)cc3)cc2)cc1 ; c1ccc(-c2ccc(-c3c(-c4cc5ccccc5c5ccccc45)cccc3N(c3ccc(-c4ccccc4)cc3)c3ccc(-c4cccc5c4oc4ccccc45)cc3)cc2)cc1]

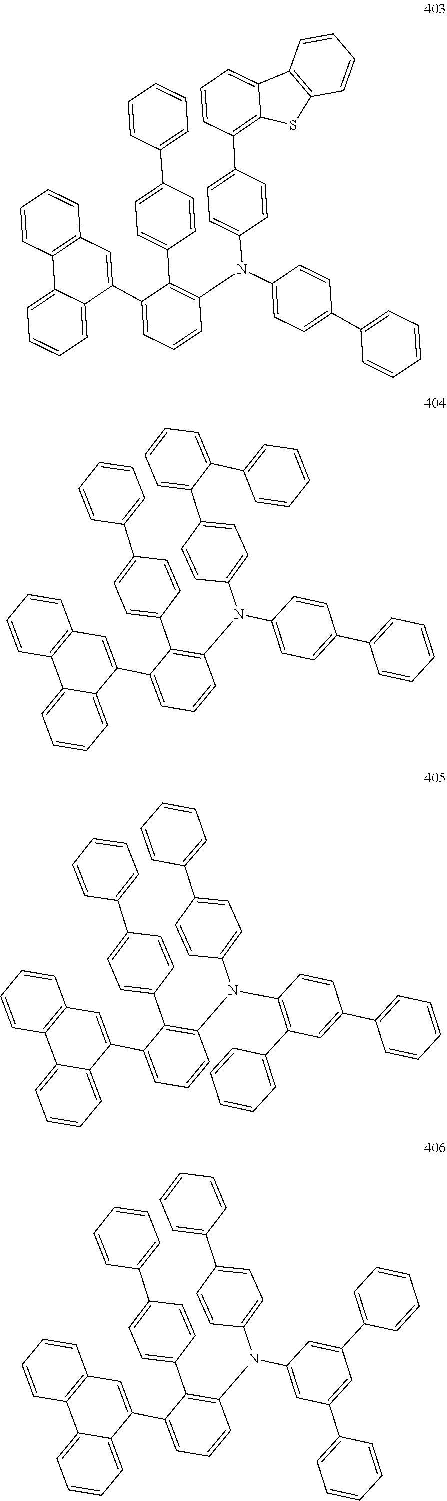 [SMILES: c1ccc(-c2ccc(-c3c(-c4cc5ccccc5c5ccccc45)cccc3N(c3ccc(-c4ccccc4)cc3)c3ccc(-c4cccc5c4sc4ccccc45)cc3)cc2)cc1 ; c1ccc(-c2ccc(-c3c(-c4cc5ccccc5c5ccccc45)cccc3N(c3ccc(-c4ccccc4)cc3)c3ccc(-c4ccccc4-c4ccccc4)cc3)cc2)cc1 ; c1ccc(-c2ccc(-c3c(-c4cc5ccccc5c5ccccc45)cccc3N(c3ccc(-c4ccccc4)cc3)c3ccc(-c4ccccc4)cc3-c3ccccc3)cc2)cc1 ; c1ccc(-c2ccc(-c3c(-c4cc5ccccc5c5ccccc45)cccc3N(c3ccc(-c4ccccc4)cc3)c3cc(-c4ccccc4)cc(-c4ccccc4)c3)cc2)cc1]

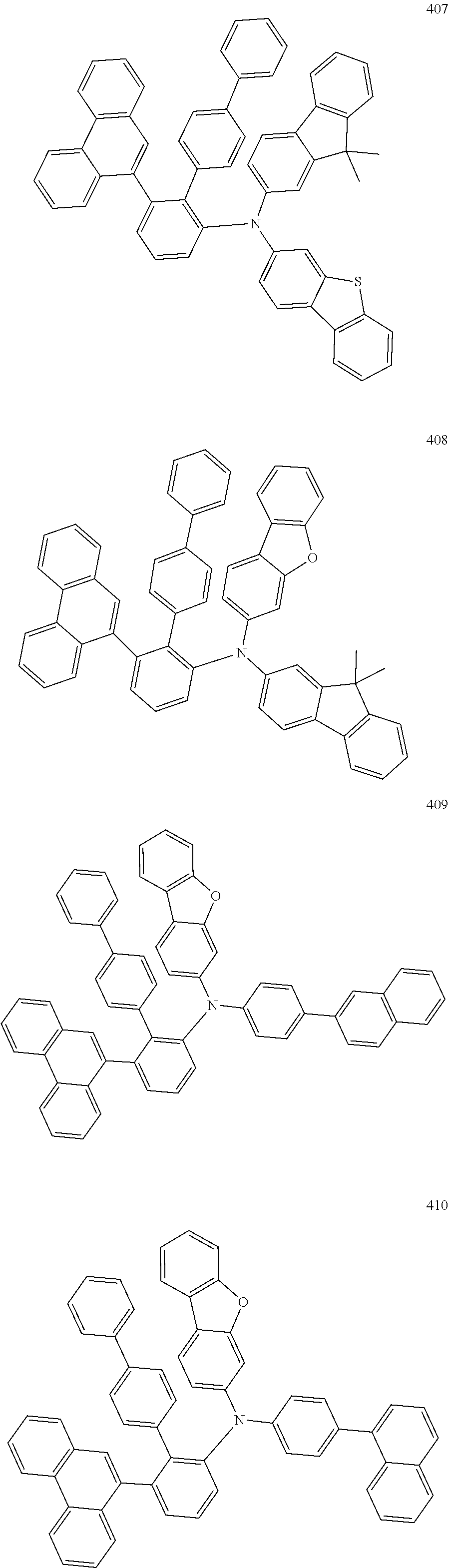 [SMILES: CC1(C)c2ccccc2-c2ccc(N(c3ccc4c(c3)sc3ccccc34)c3cccc(-c4cc5ccccc5c5ccccc45)c3-c3ccc(-c4ccccc4)cc3)cc21 ; CC1(C)c2ccccc2-c2ccc(N(c3ccc4c(c3)oc3ccccc34)c3cccc(-c4cc5ccccc5c5ccccc45)c3-c3ccc(-c4ccccc4)cc3)cc21 ; c1ccc(-c2ccc(-c3c(-c4cc5ccccc5c5ccccc45)cccc3N(c3ccc(-c4ccc5ccccc5c4)cc3)c3ccc4c(c3)oc3ccccc34)cc2)cc1 ; c1ccc(-c2ccc(-c3c(-c4cc5ccccc5c5ccccc45)cccc3N(c3ccc(-c4cccc5ccccc45)cc3)c3ccc4c(c3)oc3ccccc34)cc2)cc1]

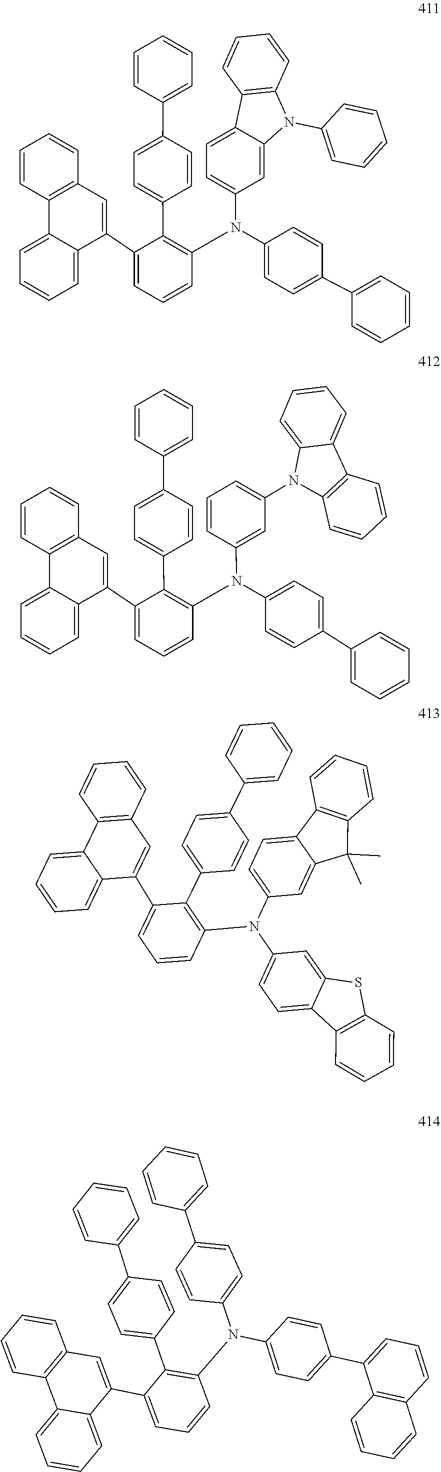 [SMILES: c1ccc(-c2ccc(-c3c(-c4cc5ccccc5c5ccccc45)cccc3N(c3ccc(-c4ccccc4)cc3)c3ccc4c5ccccc5n(-c5ccccc5)c4c3)cc2)cc1 ; c1ccc(-c2ccc(-c3c(-c4cc5ccccc5c5ccccc45)cccc3N(c3ccc(-c4ccccc4)cc3)c3cccc(-n4c5ccccc5c5ccccc54)c3)cc2)cc1 ; CC1(C)c2ccccc2-c2ccc(N(c3ccc4c(c3)sc3ccccc34)c3cccc(-c4cc5ccccc5c5ccccc45)c3-c3ccc(-c4ccccc4)cc3)cc21 ; c1ccc(-c2ccc(-c3c(-c4cc5ccccc5c5ccccc45)cccc3N(c3ccc(-c4ccccc4)cc3)c3ccc(-c4cccc5ccccc45)cc3)cc2)cc1]

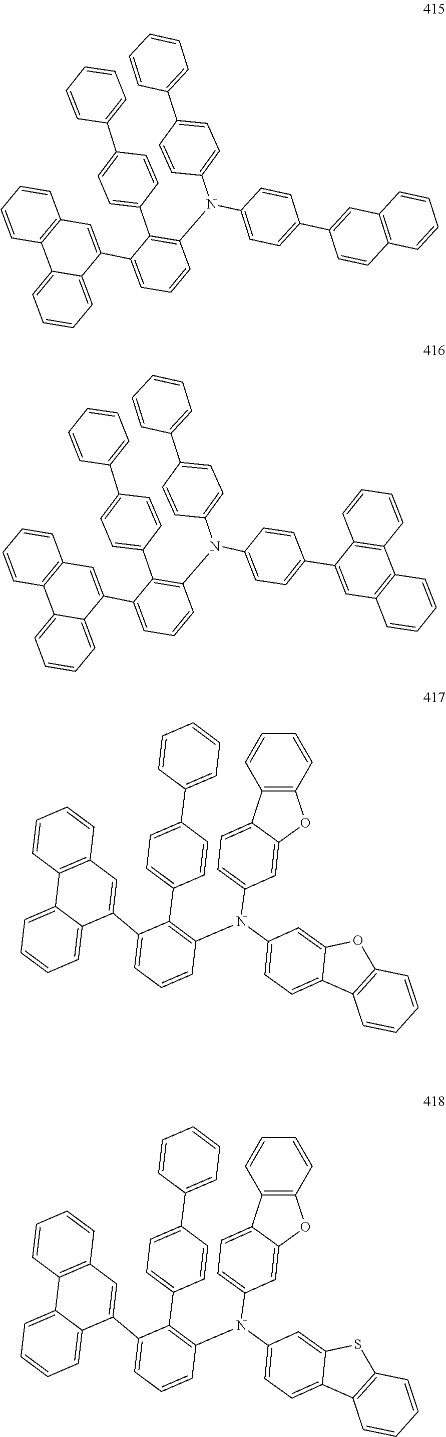 [SMILES: c1ccc(-c2ccc(-c3c(-c4cc5ccccc5c5ccccc45)cccc3N(c3ccc(-c4ccccc4)cc3)c3ccc(-c4ccc5ccccc5c4)cc3)cc2)cc1 ; c1ccc(-c2ccc(-c3c(-c4cc5ccccc5c5ccccc45)cccc3N(c3ccc(-c4ccccc4)cc3)c3ccc(-c4cc5ccccc5c5ccccc45)cc3)cc2)cc1 ; c1ccc(-c2ccc(-c3c(-c4cc5ccccc5c5ccccc45)cccc3N(c3ccc4c(c3)oc3ccccc34)c3ccc4c(c3)oc3ccccc34)cc2)cc1 ; c1ccc(-c2ccc(-c3c(-c4cc5ccccc5c5ccccc45)cccc3N(c3ccc4c(c3)oc3ccccc34)c3ccc4c(c3)sc3ccccc34)cc2)cc1]

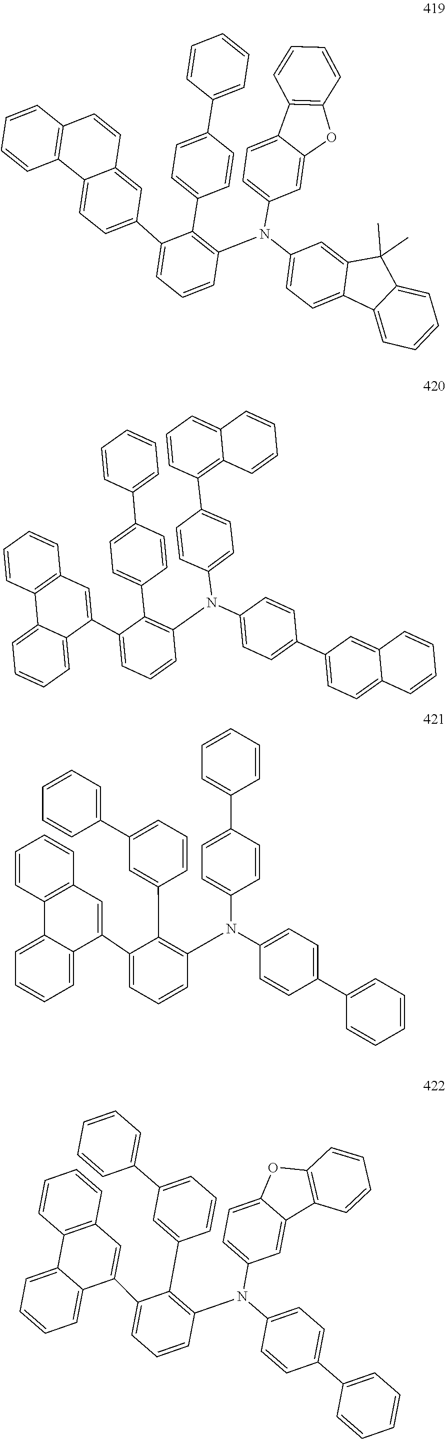 [SMILES: CC1(C)c2ccccc2-c2ccc(N(c3ccc4c(c3)oc3ccccc34)c3cccc(-c4ccc5c(ccc6ccccc65)c4)c3-c3ccc(-c4ccccc4)cc3)cc21 ; c1ccc(-c2ccc(-c3c(-c4cc5ccccc5c5ccccc45)cccc3N(c3ccc(-c4ccc5ccccc5c4)cc3)c3ccc(-c4cccc5ccccc45)cc3)cc2)cc1 ; c1ccc(-c2ccc(N(c3ccc(-c4ccccc4)cc3)c3cccc(-c4cc5ccccc5c5ccccc45)c3-c3cccc(-c4ccccc4)c3)cc2)cc1 ; c1ccc(-c2ccc(N(c3ccc4oc5ccccc5c4c3)c3cccc(-c4cc5ccccc5c5ccccc45)c3-c3cccc(-c4ccccc4)c3)cc2)cc1]

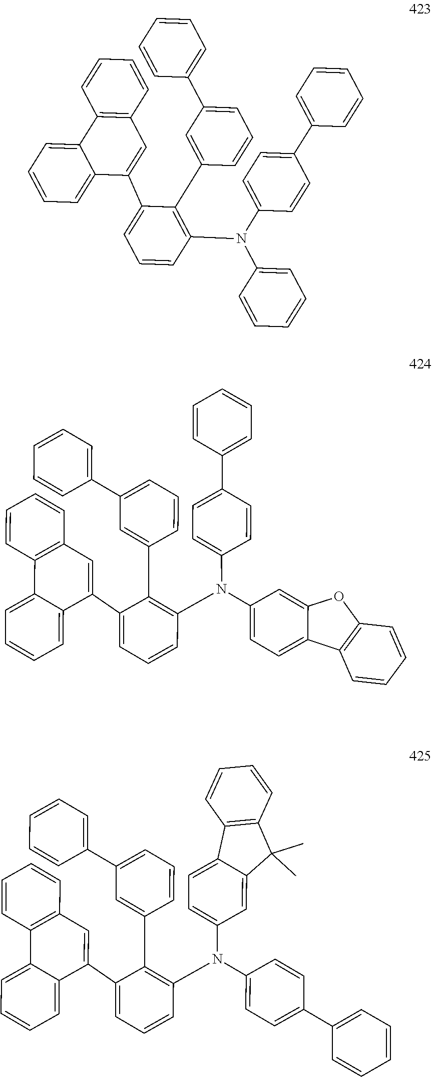 [SMILES: c1ccc(-c2ccc(N(c3ccccc3)c3cccc(-c4cc5ccccc5c5ccccc45)c3-c3cccc(-c4ccccc4)c3)cc2)cc1 ; c1ccc(-c2ccc(N(c3ccc4c(c3)oc3ccccc34)c3cccc(-c4cc5ccccc5c5ccccc45)c3-c3cccc(-c4ccccc4)c3)cc2)cc1 ; CC1(C)c2ccccc2-c2ccc(N(c3ccc(-c4ccccc4)cc3)c3cccc(-c4cc5ccccc5c5ccccc45)c3-c3cccc(-c4ccccc4)c3)cc21]

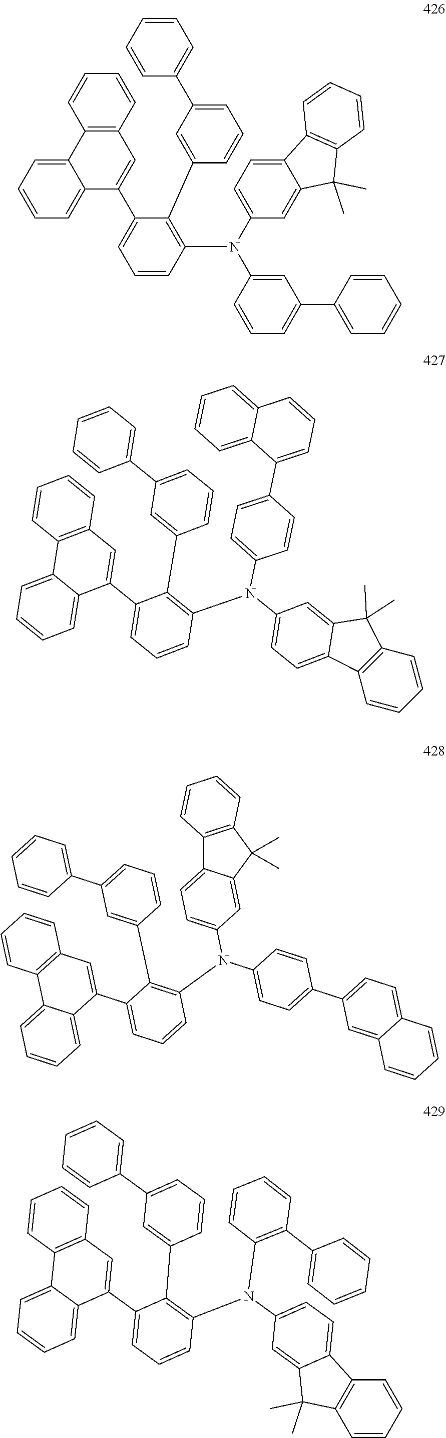 [SMILES: CC1(C)c2ccccc2-c2ccc(N(c3cccc(-c4ccccc4)c3)c3cccc(-c4cc5ccccc5c5ccccc45)c3-c3cccc(-c4ccccc4)c3)cc21 ; CC1(C)c2ccccc2-c2ccc(N(c3ccc(-c4cccc5ccccc45)cc3)c3cccc(-c4cc5ccccc5c5ccccc45)c3-c3cccc(-c4ccccc4)c3)cc21 ; CC1(C)c2ccccc2-c2ccc(N(c3ccc(-c4ccc5ccccc5c4)cc3)c3cccc(-c4cc5ccccc5c5ccccc45)c3-c3cccc(-c4ccccc4)c3)cc21 ; CC1(C)c2ccccc2-c2ccc(N(c3ccccc3-c3ccccc3)c3cccc(-c4cc5ccccc5c5ccccc45)c3-c3cccc(-c4ccccc4)c3)cc21]

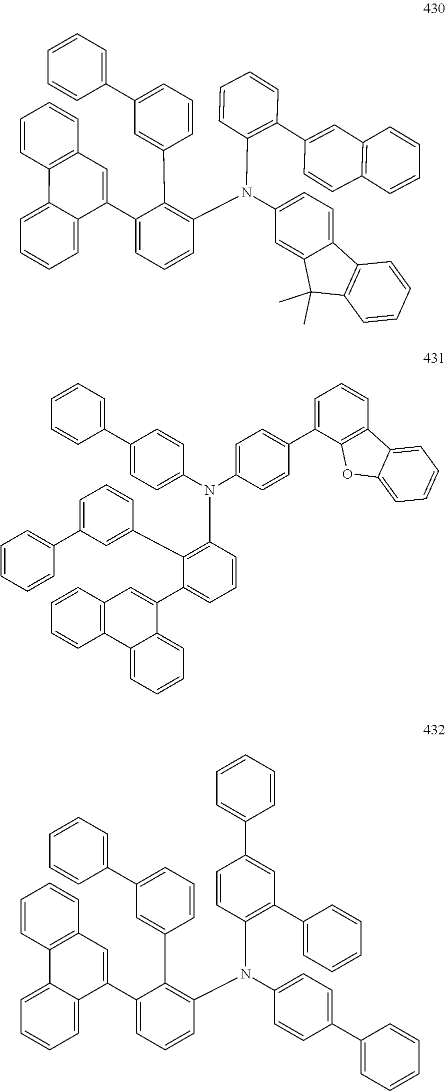 [SMILES: CC1(C)c2ccccc2-c2ccc(N(c3ccccc3-c3ccc4ccccc4c3)c3cccc(-c4cc5ccccc5c5ccccc45)c3-c3cccc(-c4ccccc4)c3)cc21 ; c1ccc(-c2ccc(N(c3ccc(-c4cccc5c4oc4ccccc45)cc3)c3cccc(-c4cc5ccccc5c5ccccc45)c3-c3cccc(-c4ccccc4)c3)cc2)cc1 ; c1ccc(-c2ccc(N(c3ccc(-c4ccccc4)cc3-c3ccccc3)c3cccc(-c4cc5ccccc5c5ccccc45)c3-c3cccc(-c4ccccc4)c3)cc2)cc1]

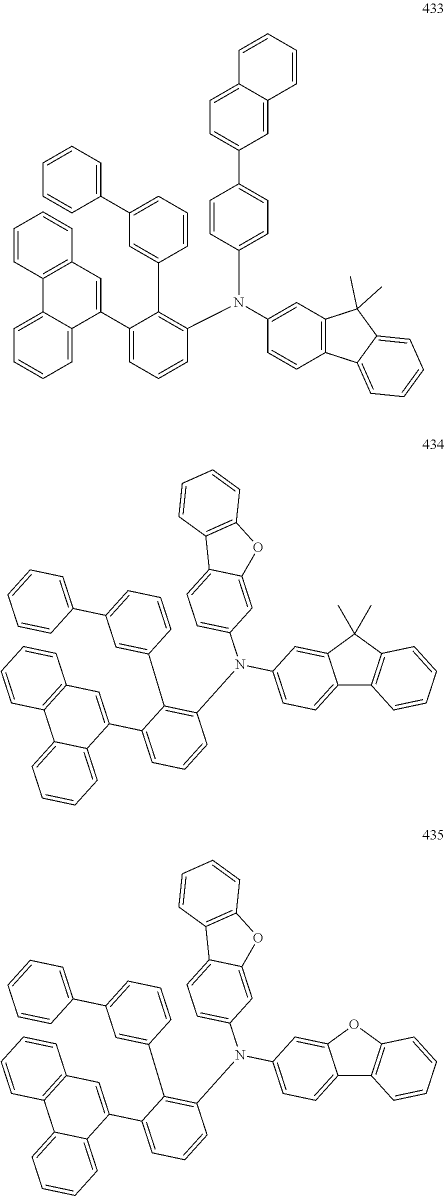 [SMILES: CC1(C)c2ccccc2-c2ccc(N(c3ccc(-c4ccc5ccccc5c4)cc3)c3cccc(-c4cc5ccccc5c5ccccc45)c3-c3cccc(-c4ccccc4)c3)cc21 ; CC1(C)c2ccccc2-c2ccc(N(c3ccc4c(c3)oc3ccccc34)c3cccc(-c4cc5ccccc5c5ccccc45)c3-c3cccc(-c4ccccc4)c3)cc21 ; c1ccc(-c2cccc(-c3c(-c4cc5ccccc5c5ccccc45)cccc3N(c3ccc4c(c3)oc3ccccc34)c3ccc4c(c3)oc3ccccc34)c2)cc1]

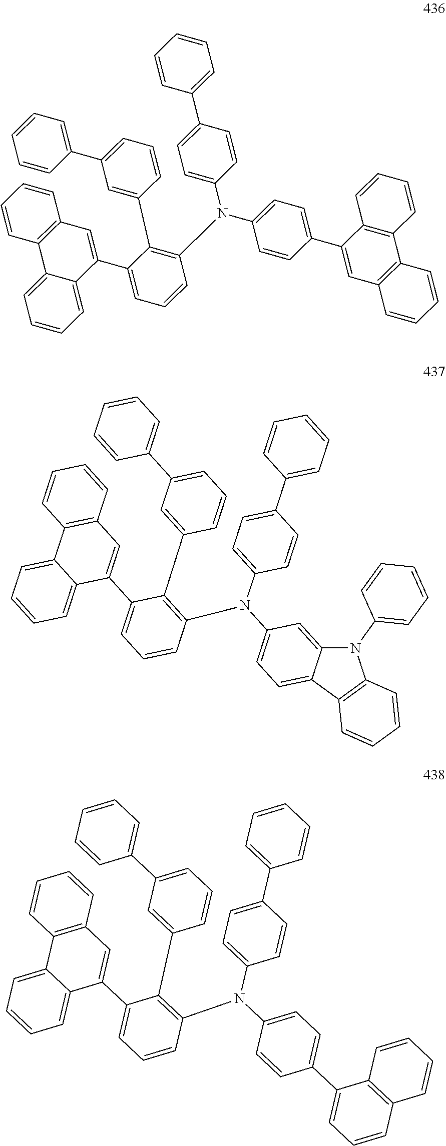 [SMILES: c1ccc(-c2ccc(N(c3ccc(-c4cc5ccccc5c5ccccc45)cc3)c3cccc(-c4cc5ccccc5c5ccccc45)c3-c3cccc(-c4ccccc4)c3)cc2)cc1 ; c1ccc(-c2ccc(N(c3ccc4c5ccccc5n(-c5ccccc5)c4c3)c3cccc(-c4cc5ccccc5c5ccccc45)c3-c3cccc(-c4ccccc4)c3)cc2)cc1 ; c1ccc(-c2ccc(N(c3ccc(-c4cccc5ccccc45)cc3)c3cccc(-c4cc5ccccc5c5ccccc45)c3-c3cccc(-c4ccccc4)c3)cc2)cc1]

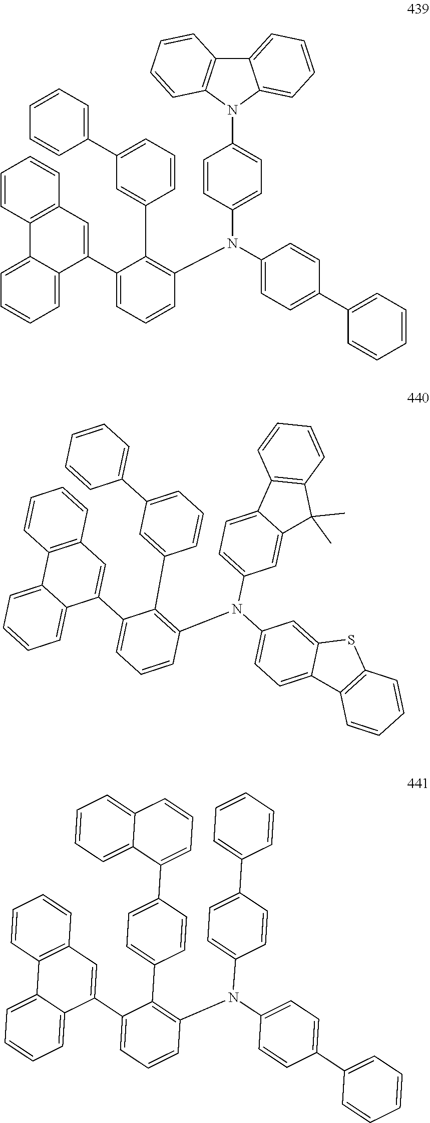 [SMILES: c1ccc(-c2ccc(N(c3ccc(-n4c5ccccc5c5ccccc54)cc3)c3cccc(-c4cc5ccccc5c5ccccc45)c3-c3cccc(-c4ccccc4)c3)cc2)cc1 ; CC1(C)c2ccccc2-c2ccc(N(c3ccc4c(c3)sc3ccccc34)c3cccc(-c4cc5ccccc5c5ccccc45)c3-c3cccc(-c4ccccc4)c3)cc21 ; c1ccc(-c2ccc(N(c3ccc(-c4ccccc4)cc3)c3cccc(-c4cc5ccccc5c5ccccc45)c3-c3ccc(-c4cccc5ccccc45)cc3)cc2)cc1]

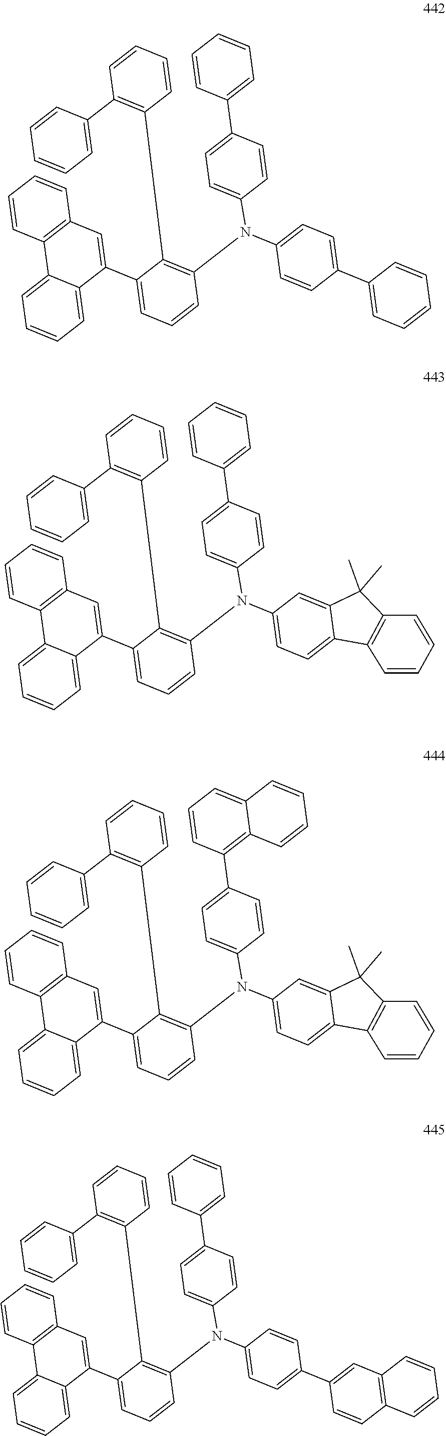 [SMILES: c1ccc(-c2ccc(N(c3ccc(-c4ccccc4)cc3)c3cccc(-c4cc5ccccc5c5ccccc45)c3-c3ccccc3-c3ccccc3)cc2)cc1 ; CC1(C)c2ccccc2-c2ccc(N(c3ccc(-c4ccccc4)cc3)c3cccc(-c4cc5ccccc5c5ccccc45)c3-c3ccccc3-c3ccccc3)cc21 ; CC1(C)c2ccccc2-c2ccc(N(c3ccc(-c4cccc5ccccc45)cc3)c3cccc(-c4cc5ccccc5c5ccccc45)c3-c3ccccc3-c3ccccc3)cc21 ; c1ccc(-c2ccc(N(c3ccc(-c4ccc5ccccc5c4)cc3)c3cccc(-c4cc5ccccc5c5ccccc45)c3-c3ccccc3-c3ccccc3)cc2)cc1]

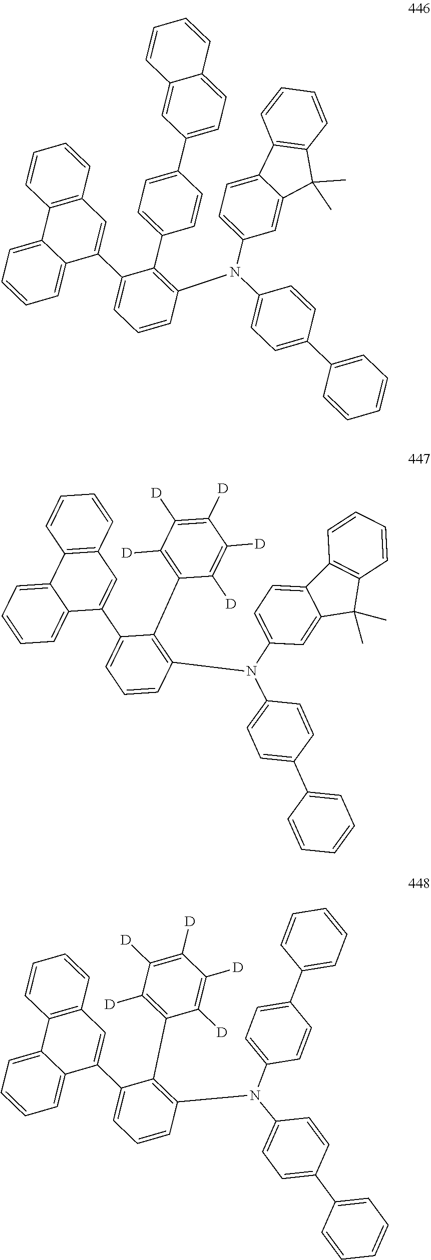 [SMILES: CC1(C)c2ccccc2-c2ccc(N(c3ccc(-c4ccccc4)cc3)c3cccc(-c4cc5ccccc5c5ccccc45)c3-c3ccc(-c4ccc5ccccc5c4)cc3)cc21 ; [2H]c1c([2H])c([2H])c(-c2c(-c3cc4ccccc4c4ccccc34)cccc2N(c2ccc(-c3ccccc3)cc2)c2ccc3c(c2)C(C)(C)c2ccccc2-3)c([2H])c1[2H] ; [2H]c1c([2H])c([2H])c(-c2c(-c3cc4ccccc4c4ccccc34)cccc2N(c2ccc(-c3ccccc3)cc2)c2ccc(-c3ccccc3)cc2)c([2H])c1[2H]]

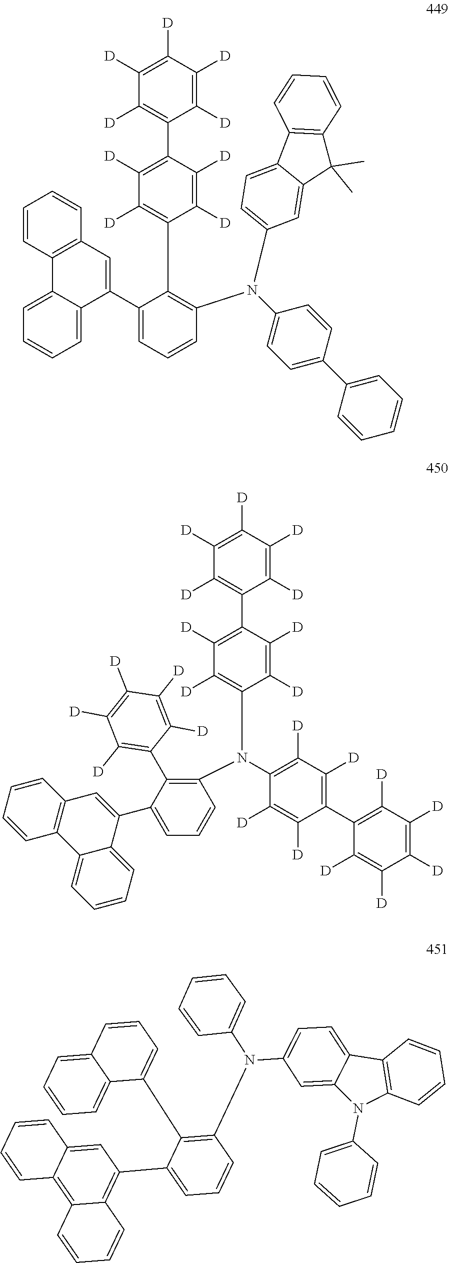 [SMILES: [2H]c1c([2H])c([2H])c(-c2c([2H])c([2H])c(-c3c(-c4cc5ccccc5c5ccccc45)cccc3N(c3ccc(-c4ccccc4)cc3)c3ccc4c(c3)C(C)(C)c3ccccc3-4)c([2H])c2[2H])c([2H])c1[2H] ; [2H]c1c([2H])c([2H])c(-c2c(-c3cc4ccccc4c4ccccc34)cccc2N(c2c([2H])c([2H])c(-c3c([2H])c([2H])c([2H])c([2H])c3[2H])c([2H])c2[2H])c2c([2H])c([2H])c(-c3c([2H])c([2H])c([2H])c([2H])c3[2H])c([2H])c2[2H])c([2H])c1[2H] ; c1ccc(N(c2ccc3c4ccccc4n(-c4ccccc4)c3c2)c2cccc(-c3cc4ccccc4c4ccccc34)c2-c2cccc3ccccc23)cc1]

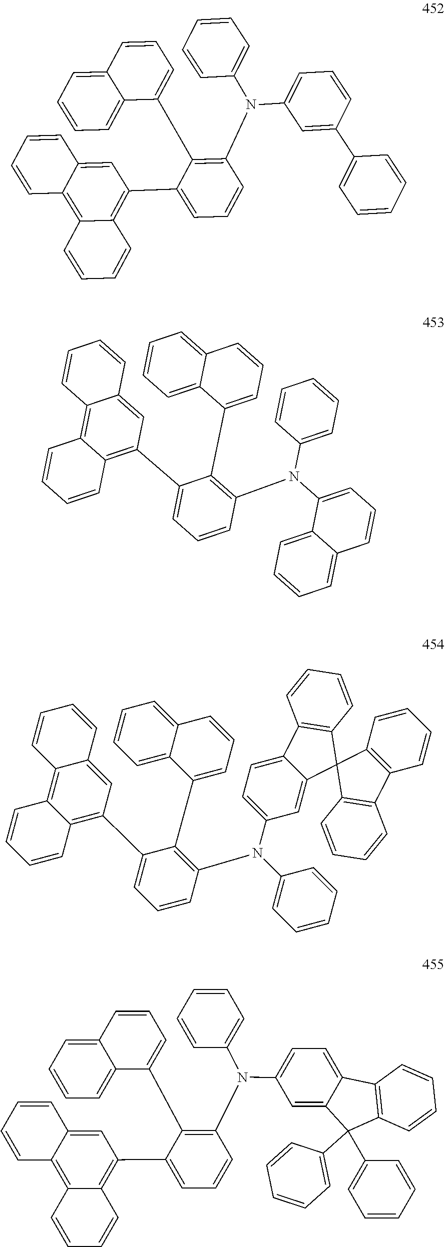 [SMILES: c1ccc(-c2cccc(N(c3ccccc3)c3cccc(-c4cc5ccccc5c5ccccc45)c3-c3cccc4ccccc34)c2)cc1 ; c1ccc(N(c2cccc(-c3cc4ccccc4c4ccccc34)c2-c2cccc3ccccc23)c2cccc3ccccc23)cc1 ; c1ccc(N(c2ccc3c(c2)C2(c4ccccc4-c4ccccc42)c2ccccc2-3)c2cccc(-c3cc4ccccc4c4ccccc34)c2-c2cccc3ccccc23)cc1 ; c1ccc(N(c2ccc3c(c2)C(c2ccccc2)(c2ccccc2)c2ccccc2-3)c2cccc(-c3cc4ccccc4c4ccccc34)c2-c2cccc3ccccc23)cc1]

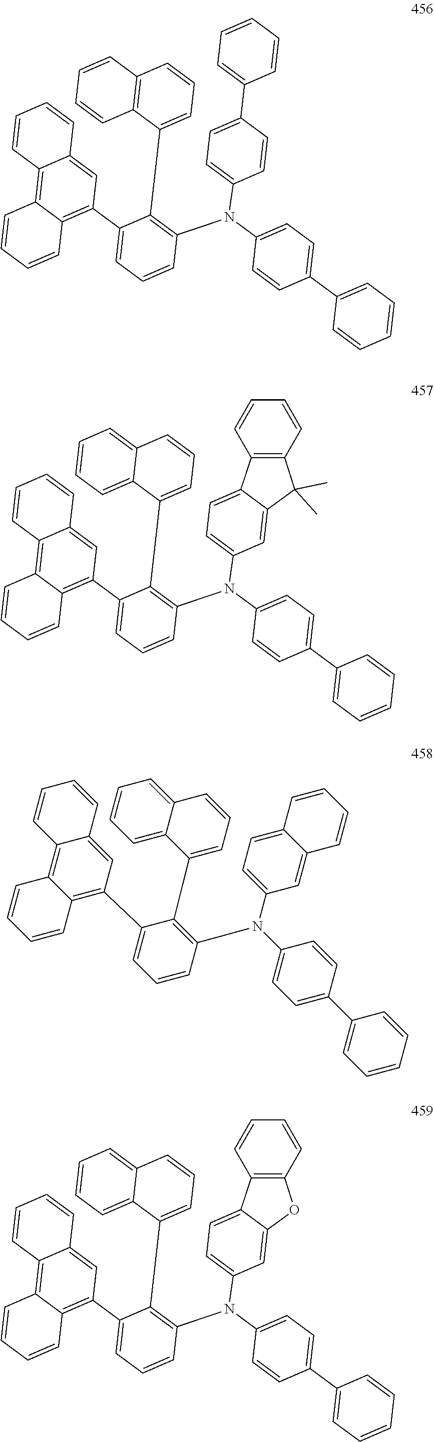 [SMILES: c1ccc(-c2ccc(N(c3ccc(-c4ccccc4)cc3)c3cccc(-c4cc5ccccc5c5ccccc45)c3-c3cccc4ccccc34)cc2)cc1 ; CC1(C)c2ccccc2-c2ccc(N(c3ccc(-c4ccccc4)cc3)c3cccc(-c4cc5ccccc5c5ccccc45)c3-c3cccc4ccccc34)cc21 ; c1ccc(-c2ccc(N(c3ccc4ccccc4c3)c3cccc(-c4cc5ccccc5c5ccccc45)c3-c3cccc4ccccc34)cc2)cc1 ; c1ccc(-c2ccc(N(c3ccc4c(c3)oc3ccccc34)c3cccc(-c4cc5ccccc5c5ccccc45)c3-c3cccc4ccccc34)cc2)cc1]

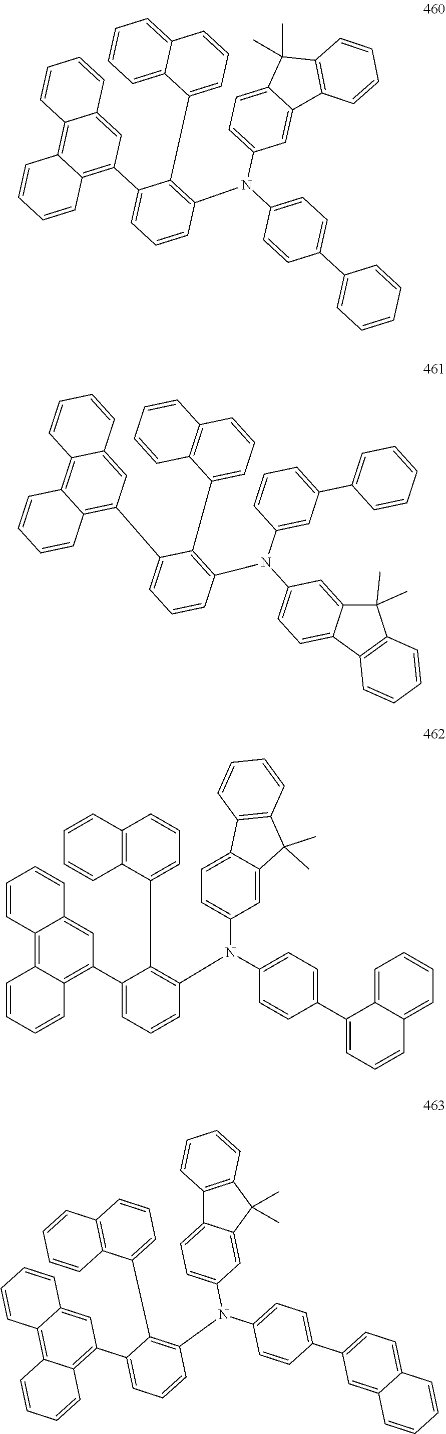 [SMILES: CC1(C)c2ccccc2-c2cc(N(c3ccc(-c4ccccc4)cc3)c3cccc(-c4cc5ccccc5c5ccccc45)c3-c3cccc4ccccc34)ccc21 ; CC1(C)c2ccccc2-c2ccc(N(c3cccc(-c4ccccc4)c3)c3cccc(-c4cc5ccccc5c5ccccc45)c3-c3cccc4ccccc34)cc21 ; CC1(C)c2ccccc2-c2ccc(N(c3ccc(-c4cccc5ccccc45)cc3)c3cccc(-c4cc5ccccc5c5ccccc45)c3-c3cccc4ccccc34)cc21 ; CC1(C)c2ccccc2-c2ccc(N(c3ccc(-c4ccc5ccccc5c4)cc3)c3cccc(-c4cc5ccccc5c5ccccc45)c3-c3cccc4ccccc34)cc21]

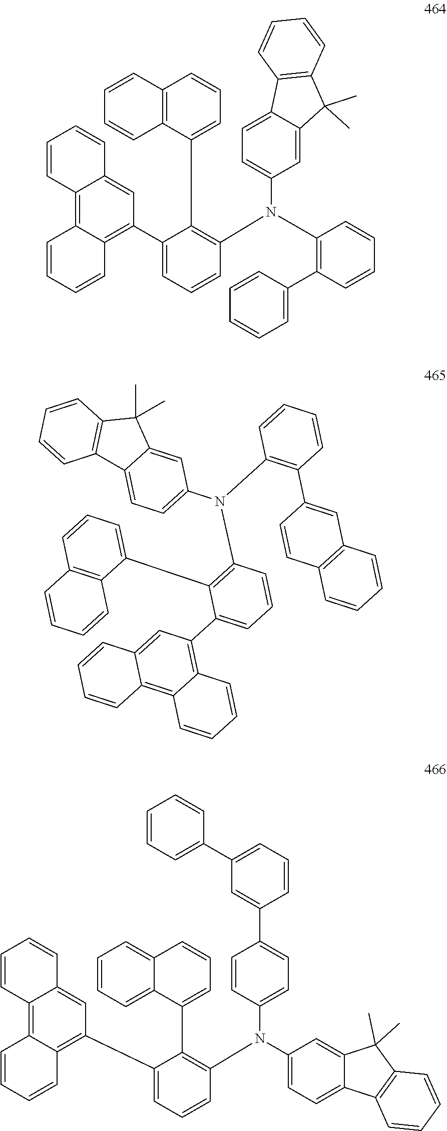 [SMILES: CC1(C)c2ccccc2-c2ccc(N(c3ccccc3-c3ccccc3)c3cccc(-c4cc5ccccc5c5ccccc45)c3-c3cccc4ccccc34)cc21 ; CC1(C)c2ccccc2-c2ccc(N(c3ccccc3-c3ccc4ccccc4c3)c3cccc(-c4cc5ccccc5c5ccccc45)c3-c3cccc4ccccc34)cc21 ; CC1(C)c2ccccc2-c2ccc(N(c3ccc(-c4cccc(-c5ccccc5)c4)cc3)c3cccc(-c4cc5ccccc5c5ccccc45)c3-c3cccc4ccccc34)cc21]

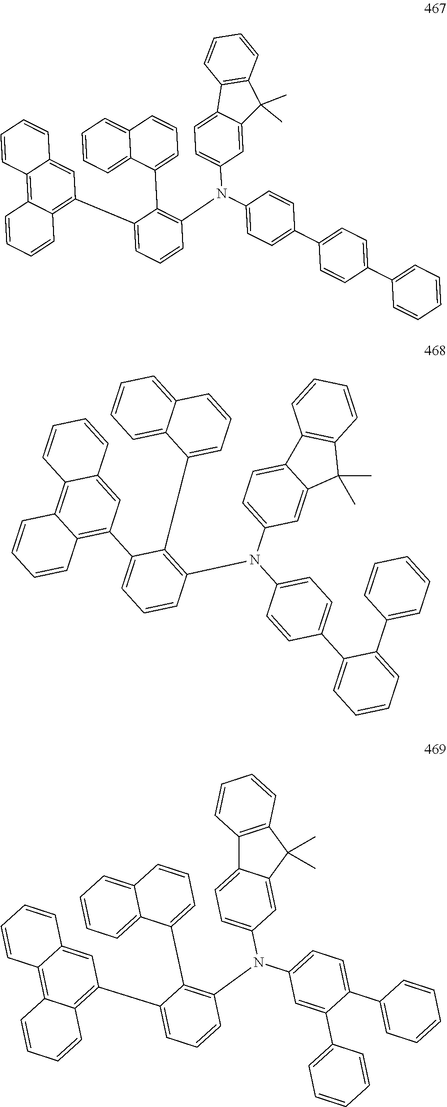 [SMILES: CC1(C)c2ccccc2-c2ccc(N(c3ccc(-c4ccc(-c5ccccc5)cc4)cc3)c3cccc(-c4cc5ccccc5c5ccccc45)c3-c3cccc4ccccc34)cc21 ; CC1(C)c2ccccc2-c2ccc(N(c3ccc(-c4ccccc4-c4ccccc4)cc3)c3cccc(-c4cc5ccccc5c5ccccc45)c3-c3cccc4ccccc34)cc21 ; CC1(C)c2ccccc2-c2ccc(N(c3ccc(-c4ccccc4)c(-c4ccccc4)c3)c3cccc(-c4cc5ccccc5c5ccccc45)c3-c3cccc4ccccc34)cc21]

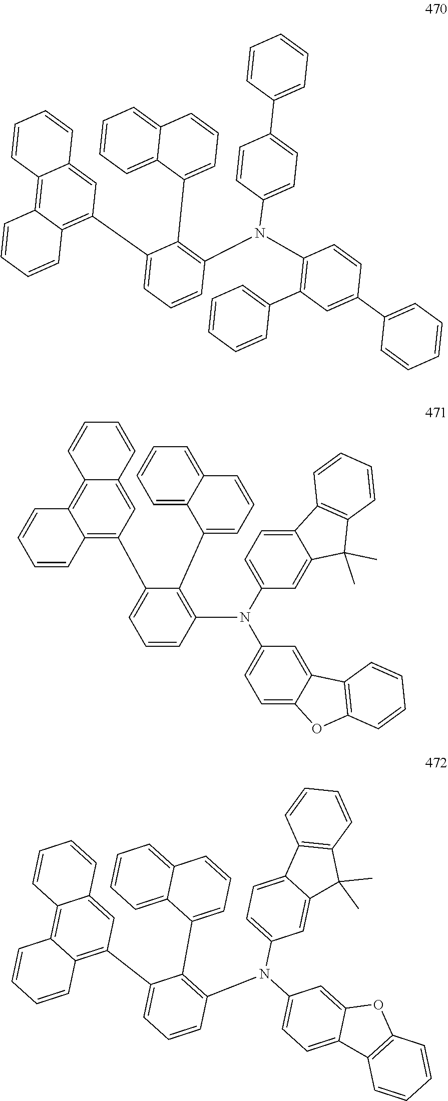 [SMILES: c1ccc(-c2ccc(N(c3ccc(-c4ccccc4)cc3-c3ccccc3)c3cccc(-c4cc5ccccc5c5ccccc45)c3-c3cccc4ccccc34)cc2)cc1 ; CC1(C)c2ccccc2-c2ccc(N(c3ccc4oc5ccccc5c4c3)c3cccc(-c4cc5ccccc5c5ccccc45)c3-c3cccc4ccccc34)cc21 ; CC1(C)c2ccccc2-c2ccc(N(c3ccc4c(c3)oc3ccccc34)c3cccc(-c4cc5ccccc5c5ccccc45)c3-c3cccc4ccccc34)cc21]

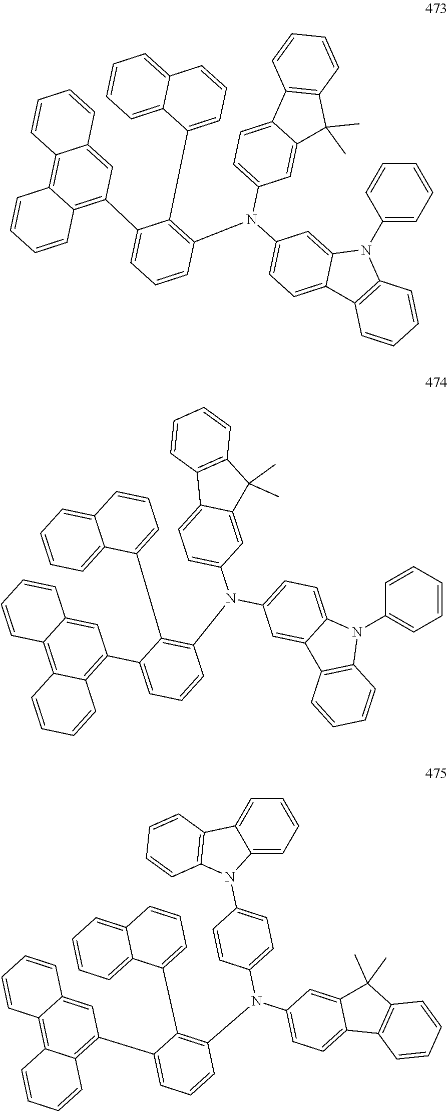 [SMILES: CC1(C)c2ccccc2-c2ccc(N(c3ccc4c5ccccc5n(-c5ccccc5)c4c3)c3cccc(-c4cc5ccccc5c5ccccc45)c3-c3cccc4ccccc34)cc21 ; CC1(C)c2ccccc2-c2ccc(N(c3ccc4c(c3)c3ccccc3n4-c3ccccc3)c3cccc(-c4cc5ccccc5c5ccccc45)c3-c3cccc4ccccc34)cc21 ; CC1(C)c2ccccc2-c2ccc(N(c3ccc(-n4c5ccccc5c5ccccc54)cc3)c3cccc(-c4cc5ccccc5c5ccccc45)c3-c3cccc4ccccc34)cc21]

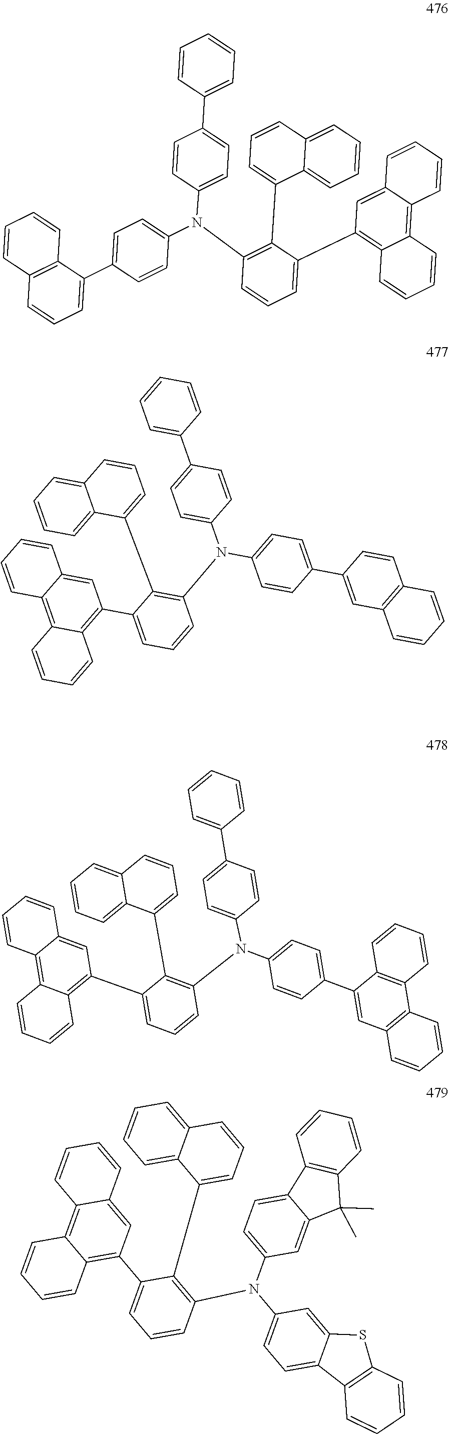 [SMILES: c1ccc(-c2ccc(N(c3ccc(-c4cccc5ccccc45)cc3)c3cccc(-c4cc5ccccc5c5ccccc45)c3-c3cccc4ccccc34)cc2)cc1 ; c1ccc(-c2ccc(N(c3ccc(-c4ccc5ccccc5c4)cc3)c3cccc(-c4cc5ccccc5c5ccccc45)c3-c3cccc4ccccc34)cc2)cc1 ; c1ccc(-c2ccc(N(c3ccc(-c4cc5ccccc5c5ccccc45)cc3)c3cccc(-c4cc5ccccc5c5ccccc45)c3-c3cccc4ccccc34)cc2)cc1 ; CC1(C)c2ccccc2-c2ccc(N(c3ccc4c(c3)sc3ccccc34)c3cccc(-c4cc5ccccc5c5ccccc45)c3-c3cccc4ccccc34)cc21]

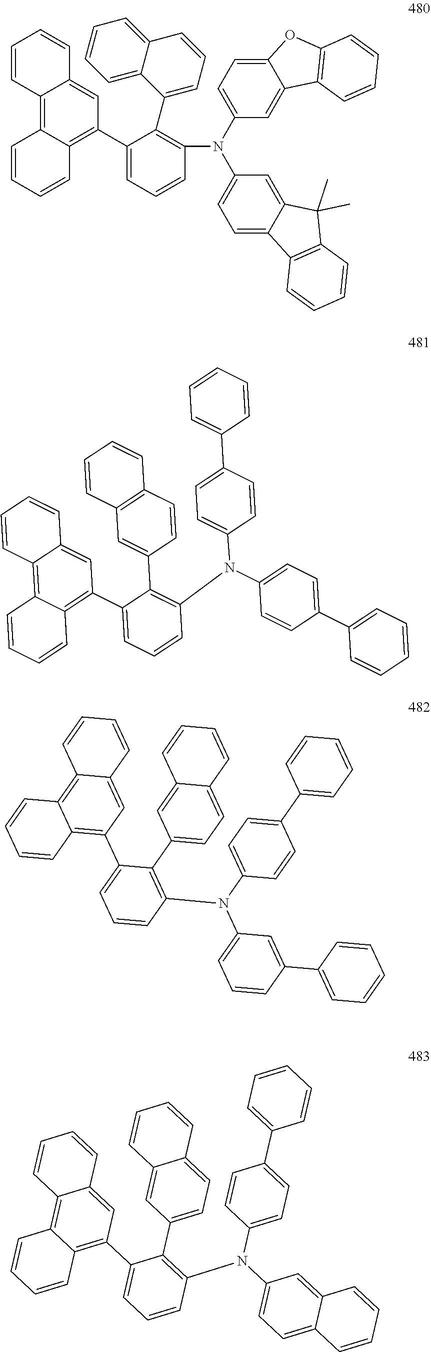 [SMILES: CC1(C)c2ccccc2-c2ccc(N(c3ccc4oc5ccccc5c4c3)c3cccc(-c4cc5ccccc5c5ccccc45)c3-c3cccc4ccccc34)cc21 ; c1ccc(-c2ccc(N(c3ccc(-c4ccccc4)cc3)c3cccc(-c4cc5ccccc5c5ccccc45)c3-c3ccc4ccccc4c3)cc2)cc1 ; c1ccc(-c2ccc(N(c3cccc(-c4ccccc4)c3)c3cccc(-c4cc5ccccc5c5ccccc45)c3-c3ccc4ccccc4c3)cc2)cc1 ; c1ccc(-c2ccc(N(c3ccc4ccccc4c3)c3cccc(-c4cc5ccccc5c5ccccc45)c3-c3ccc4ccccc4c3)cc2)cc1]

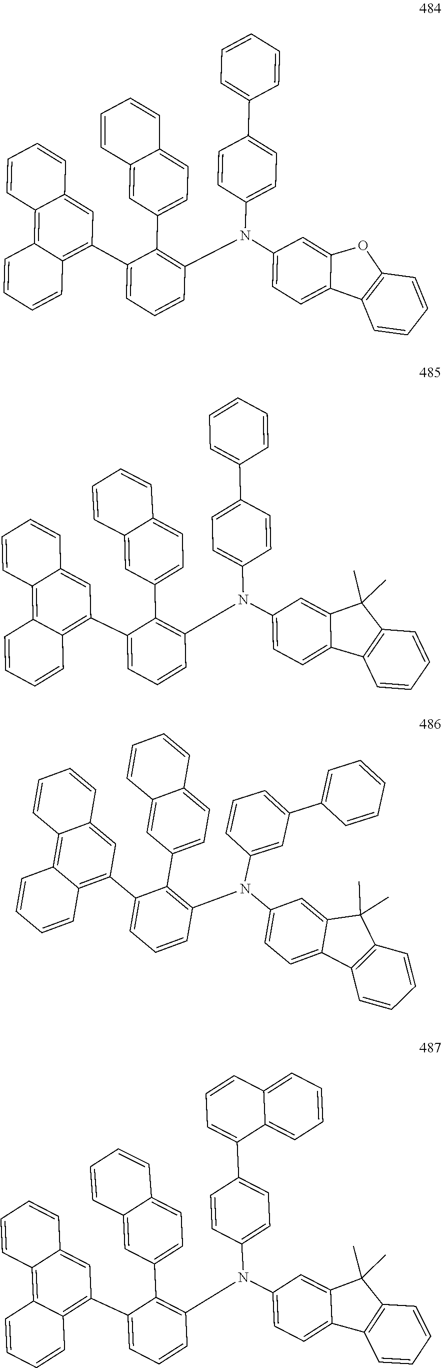 [SMILES: c1ccc(-c2ccc(N(c3ccc4c(c3)oc3ccccc34)c3cccc(-c4cc5ccccc5c5ccccc45)c3-c3ccc4ccccc4c3)cc2)cc1 ; CC1(C)c2ccccc2-c2ccc(N(c3ccc(-c4ccccc4)cc3)c3cccc(-c4cc5ccccc5c5ccccc45)c3-c3ccc4ccccc4c3)cc21 ; CC1(C)c2ccccc2-c2ccc(N(c3cccc(-c4ccccc4)c3)c3cccc(-c4cc5ccccc5c5ccccc45)c3-c3ccc4ccccc4c3)cc21 ; CC1(C)c2ccccc2-c2ccc(N(c3ccc(-c4cccc5ccccc45)cc3)c3cccc(-c4cc5ccccc5c5ccccc45)c3-c3ccc4ccccc4c3)cc21]

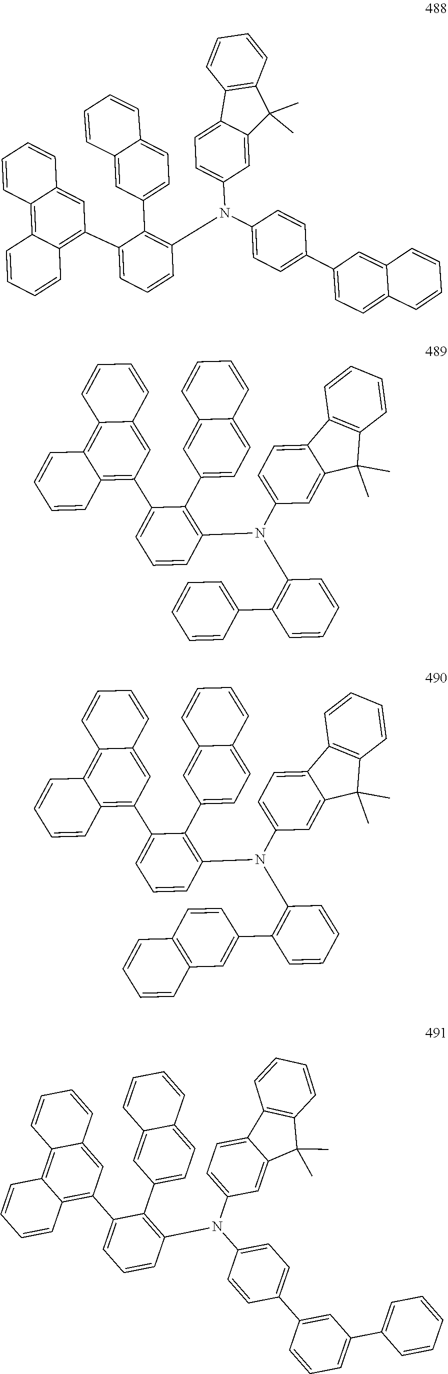 [SMILES: CC1(C)c2ccccc2-c2ccc(N(c3ccc(-c4ccc5ccccc5c4)cc3)c3cccc(-c4cc5ccccc5c5ccccc45)c3-c3ccc4ccccc4c3)cc21 ; CC1(C)c2ccccc2-c2ccc(N(c3ccccc3-c3ccccc3)c3cccc(-c4cc5ccccc5c5ccccc45)c3-c3ccc4ccccc4c3)cc21 ; CC1(C)c2ccccc2-c2ccc(N(c3ccccc3-c3ccc4ccccc4c3)c3cccc(-c4cc5ccccc5c5ccccc45)c3-c3ccc4ccccc4c3)cc21 ; CC1(C)c2ccccc2-c2ccc(N(c3ccc(-c4cccc(-c5ccccc5)c4)cc3)c3cccc(-c4cc5ccccc5c5ccccc45)c3-c3ccc4ccccc4c3)cc21]

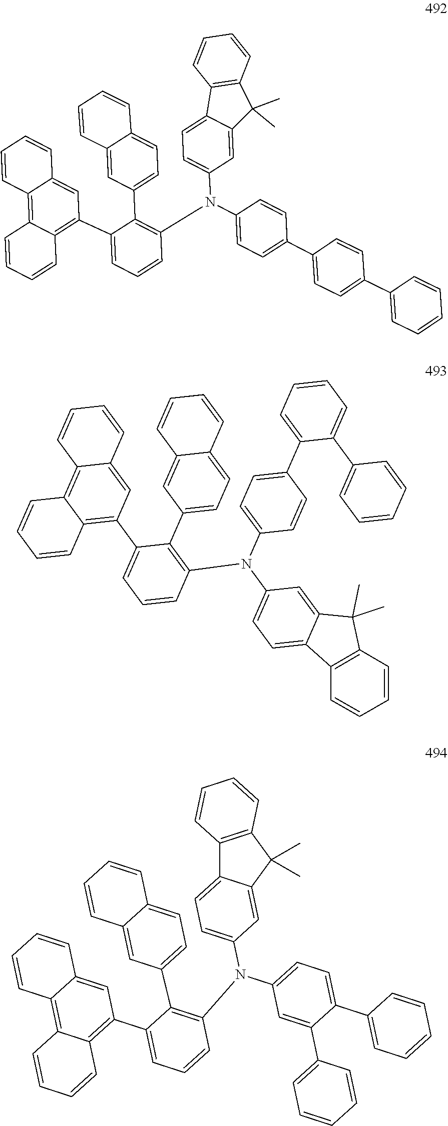 [SMILES: CC1(C)c2ccccc2-c2ccc(N(c3ccc(-c4ccc(-c5ccccc5)cc4)cc3)c3cccc(-c4cc5ccccc5c5ccccc45)c3-c3ccc4ccccc4c3)cc21 ; CC1(C)c2ccccc2-c2ccc(N(c3ccc(-c4ccccc4-c4ccccc4)cc3)c3cccc(-c4cc5ccccc5c5ccccc45)c3-c3ccc4ccccc4c3)cc21 ; CC1(C)c2ccccc2-c2ccc(N(c3ccc(-c4ccccc4)c(-c4ccccc4)c3)c3cccc(-c4cc5ccccc5c5ccccc45)c3-c3ccc4ccccc4c3)cc21]

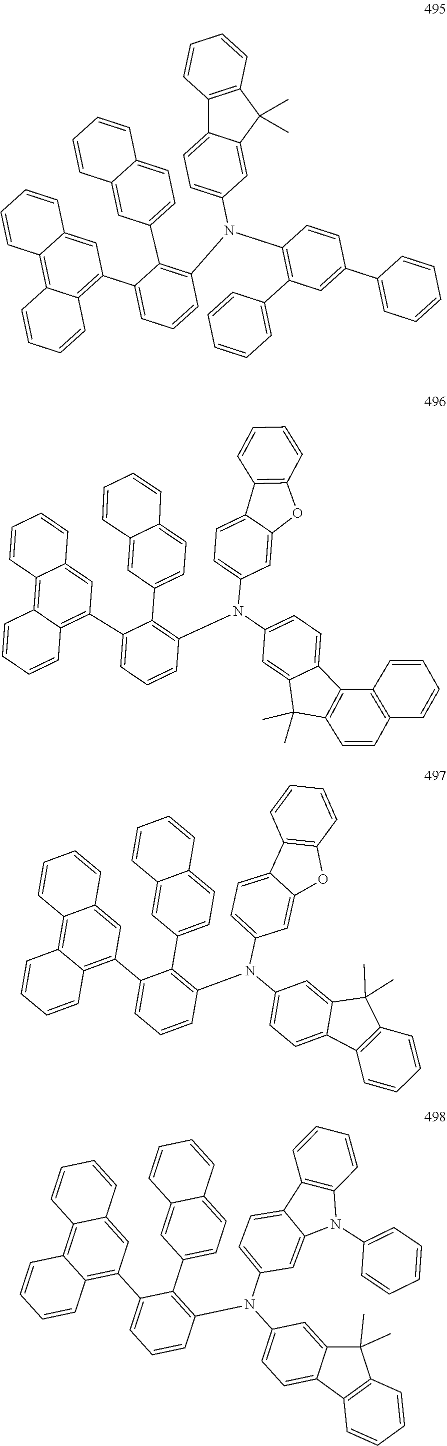

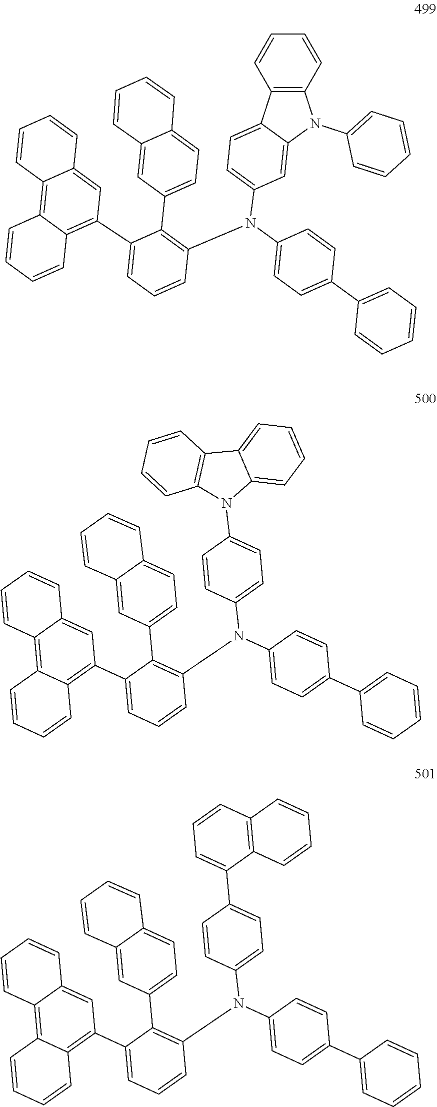 [SMILES: c1ccc(-c2ccc(N(c3ccc4c5ccccc5n(-c5ccccc5)c4c3)c3cccc(-c4cc5ccccc5c5ccccc45)c3-c3ccc4ccccc4c3)cc2)cc1 ; c1ccc(-c2ccc(N(c3ccc(-n4c5ccccc5c5ccccc54)cc3)c3cccc(-c4cc5ccccc5c5ccccc45)c3-c3ccc4ccccc4c3)cc2)cc1 ; c1ccc(-c2ccc(N(c3ccc(-c4cccc5ccccc45)cc3)c3cccc(-c4cc5ccccc5c5ccccc45)c3-c3ccc4ccccc4c3)cc2)cc1]

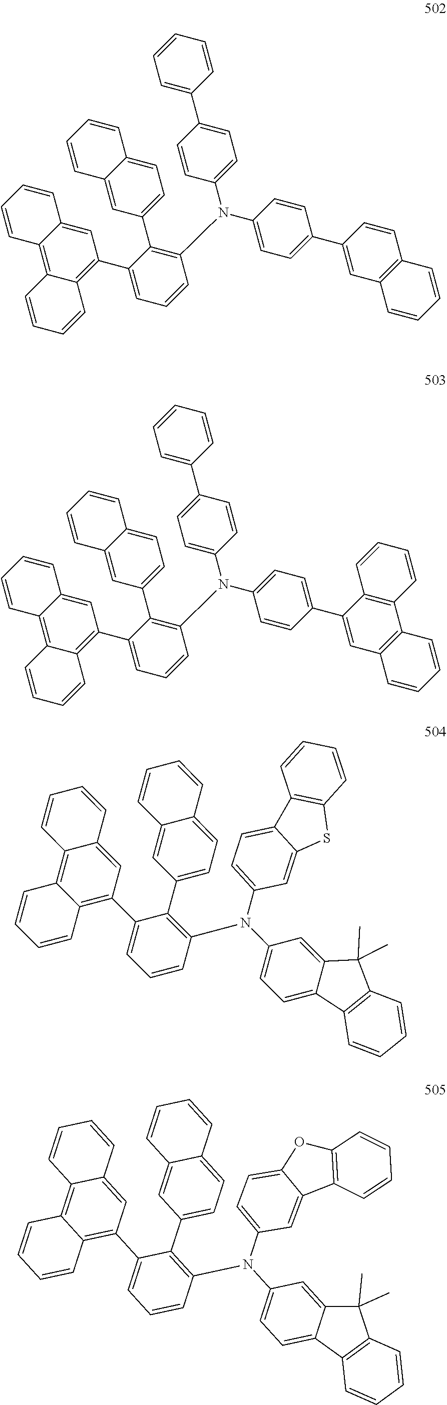 [SMILES: c1ccc(-c2ccc(N(c3ccc(-c4ccc5ccccc5c4)cc3)c3cccc(-c4cc5ccccc5c5ccccc45)c3-c3ccc4ccccc4c3)cc2)cc1 ; c1ccc(-c2ccc(N(c3ccc(-c4cc5ccccc5c5ccccc45)cc3)c3cccc(-c4cc5ccccc5c5ccccc45)c3-c3ccc4ccccc4c3)cc2)cc1 ; CC1(C)c2ccccc2-c2ccc(N(c3ccc4c(c3)sc3ccccc34)c3cccc(-c4cc5ccccc5c5ccccc45)c3-c3ccc4ccccc4c3)cc21 ; CC1(C)c2ccccc2-c2ccc(N(c3ccc4oc5ccccc5c4c3)c3cccc(-c4cc5ccccc5c5ccccc45)c3-c3ccc4ccccc4c3)cc21]

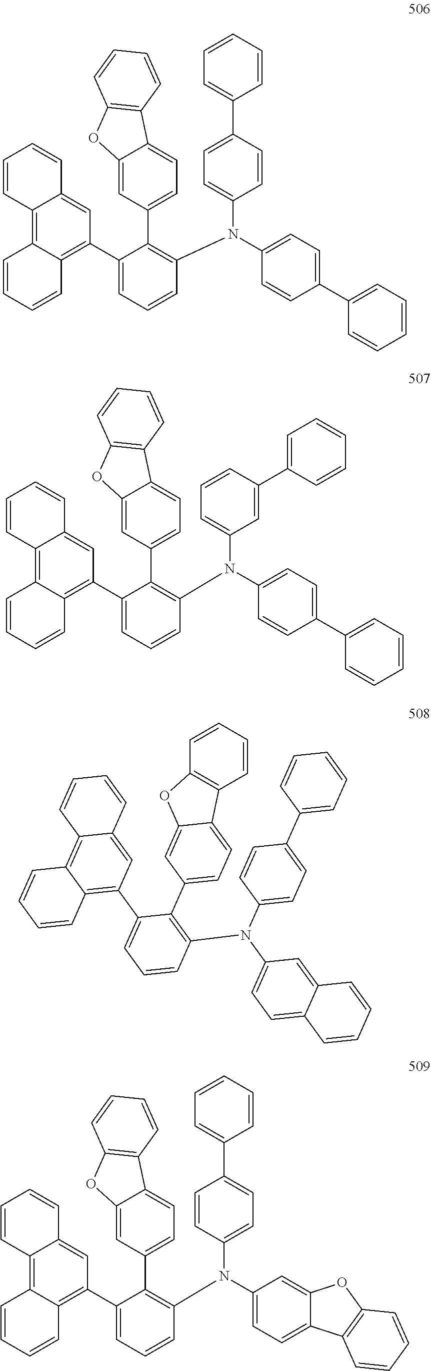 [SMILES: c1ccc(-c2ccc(N(c3ccc(-c4ccccc4)cc3)c3cccc(-c4cc5ccccc5c5ccccc45)c3-c3ccc4c(c3)oc3ccccc34)cc2)cc1 ; c1ccc(-c2ccc(N(c3cccc(-c4ccccc4)c3)c3cccc(-c4cc5ccccc5c5ccccc45)c3-c3ccc4c(c3)oc3ccccc34)cc2)cc1 ; c1ccc(-c2ccc(N(c3ccc4ccccc4c3)c3cccc(-c4cc5ccccc5c5ccccc45)c3-c3ccc4c(c3)oc3ccccc34)cc2)cc1 ; c1ccc(-c2ccc(N(c3ccc4c(c3)oc3ccccc34)c3cccc(-c4cc5ccccc5c5ccccc45)c3-c3ccc4c(c3)oc3ccccc34)cc2)cc1]

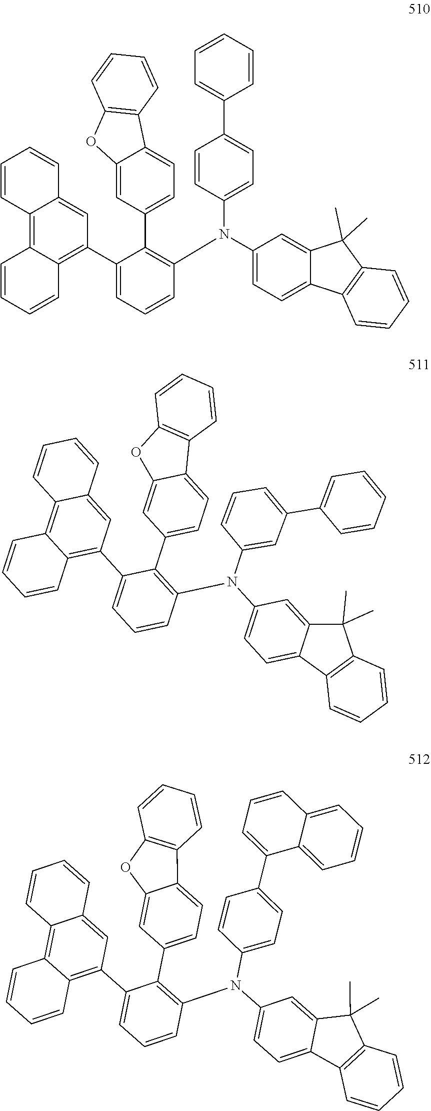 [SMILES: CC1(C)c2ccccc2-c2ccc(N(c3ccc(-c4ccccc4)cc3)c3cccc(-c4cc5ccccc5c5ccccc45)c3-c3ccc4c(c3)oc3ccccc34)cc21 ; CC1(C)c2ccccc2-c2ccc(N(c3cccc(-c4ccccc4)c3)c3cccc(-c4cc5ccccc5c5ccccc45)c3-c3ccc4c(c3)oc3ccccc34)cc21 ; CC1(C)c2ccccc2-c2ccc(N(c3ccc(-c4cccc5ccccc45)cc3)c3cccc(-c4cc5ccccc5c5ccccc45)c3-c3ccc4c(c3)oc3ccccc34)cc21]

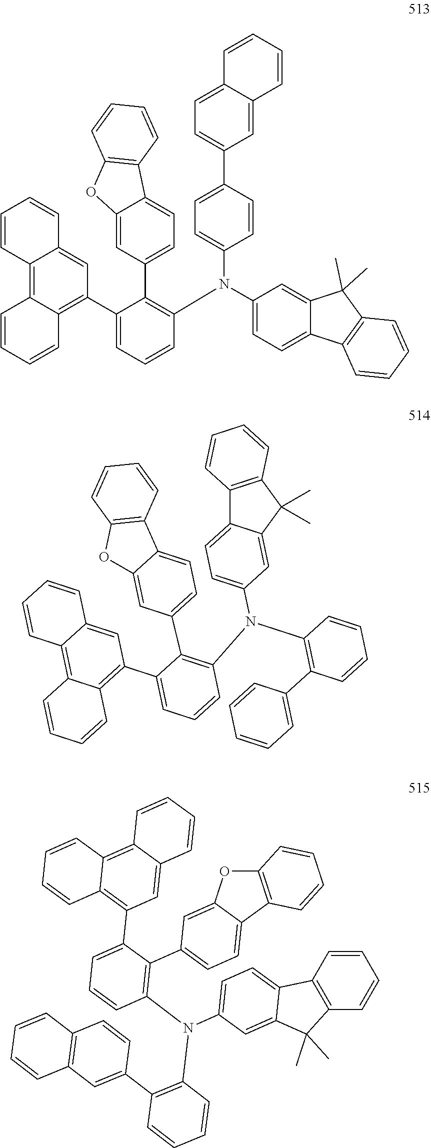 [SMILES: CC1(C)c2ccccc2-c2ccc(N(c3ccc(-c4ccc5ccccc5c4)cc3)c3cccc(-c4cc5ccccc5c5ccccc45)c3-c3ccc4c(c3)oc3ccccc34)cc21 ; CC1(C)c2ccccc2-c2ccc(N(c3ccccc3-c3ccccc3)c3cccc(-c4cc5ccccc5c5ccccc45)c3-c3ccc4c(c3)oc3ccccc34)cc21 ; CC1(C)c2ccccc2-c2ccc(N(c3ccccc3-c3ccc4ccccc4c3)c3cccc(-c4cc5ccccc5c5ccccc45)c3-c3ccc4c(c3)oc3ccccc34)cc21]

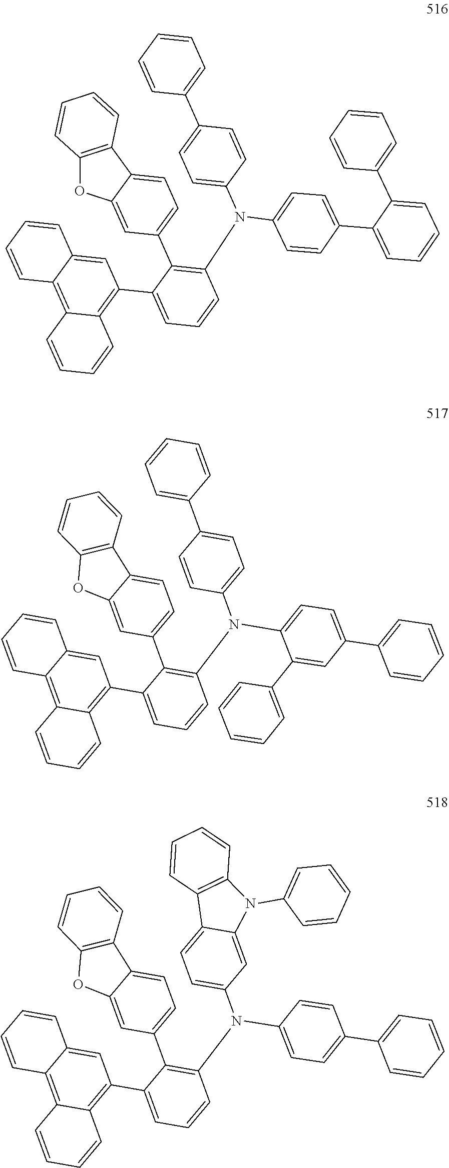 [SMILES: c1ccc(-c2ccc(N(c3ccc(-c4ccccc4-c4ccccc4)cc3)c3cccc(-c4cc5ccccc5c5ccccc45)c3-c3ccc4c(c3)oc3ccccc34)cc2)cc1 ; c1ccc(-c2ccc(N(c3ccc(-c4ccccc4)cc3-c3ccccc3)c3cccc(-c4cc5ccccc5c5ccccc45)c3-c3ccc4c(c3)oc3ccccc34)cc2)cc1 ; c1ccc(-c2ccc(N(c3ccc4c5ccccc5n(-c5ccccc5)c4c3)c3cccc(-c4cc5ccccc5c5ccccc45)c3-c3ccc4c(c3)oc3ccccc34)cc2)cc1]

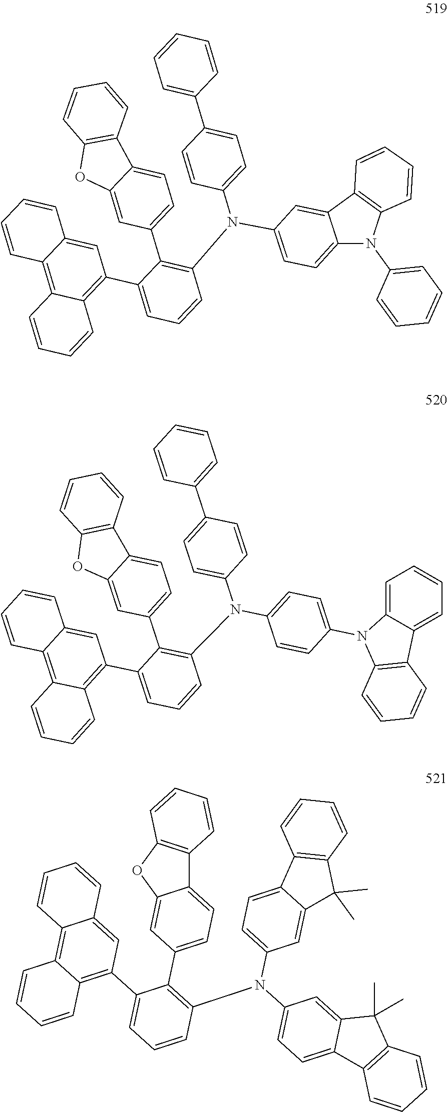 [SMILES: c1ccc(-c2ccc(N(c3ccc4c(c3)c3ccccc3n4-c3ccccc3)c3cccc(-c4cc5ccccc5c5ccccc45)c3-c3ccc4c(c3)oc3ccccc34)cc2)cc1 ; c1ccc(-c2ccc(N(c3ccc(-n4c5ccccc5c5ccccc54)cc3)c3cccc(-c4cc5ccccc5c5ccccc45)c3-c3ccc4c(c3)oc3ccccc34)cc2)cc1 ; CC1(C)c2ccccc2-c2ccc(N(c3ccc4c(c3)C(C)(C)c3ccccc3-4)c3cccc(-c4cc5ccccc5c5ccccc45)c3-c3ccc4c(c3)oc3ccccc34)cc21]

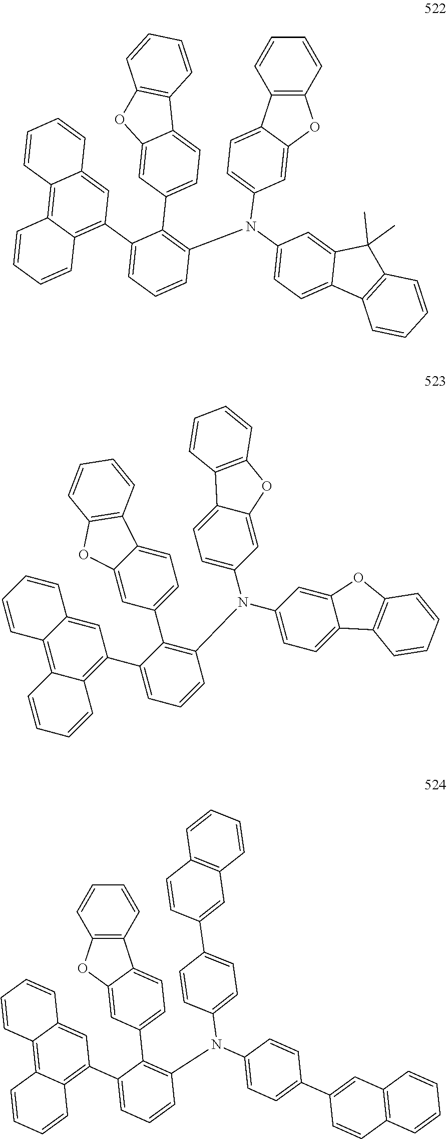 [SMILES: CC1(C)c2ccccc2-c2ccc(N(c3ccc4c(c3)oc3ccccc34)c3cccc(-c4cc5ccccc5c5ccccc45)c3-c3ccc4c(c3)oc3ccccc34)cc21 ; c1cc(-c2cc3ccccc3c3ccccc23)c(-c2ccc3c(c2)oc2ccccc23)c(N(c2ccc3c(c2)oc2ccccc23)c2ccc3c(c2)oc2ccccc23)c1 ; c1cc(-c2cc3ccccc3c3ccccc23)c(-c2ccc3c(c2)oc2ccccc23)c(N(c2ccc(-c3ccc4ccccc4c3)cc2)c2ccc(-c3ccc4ccccc4c3)cc2)c1]

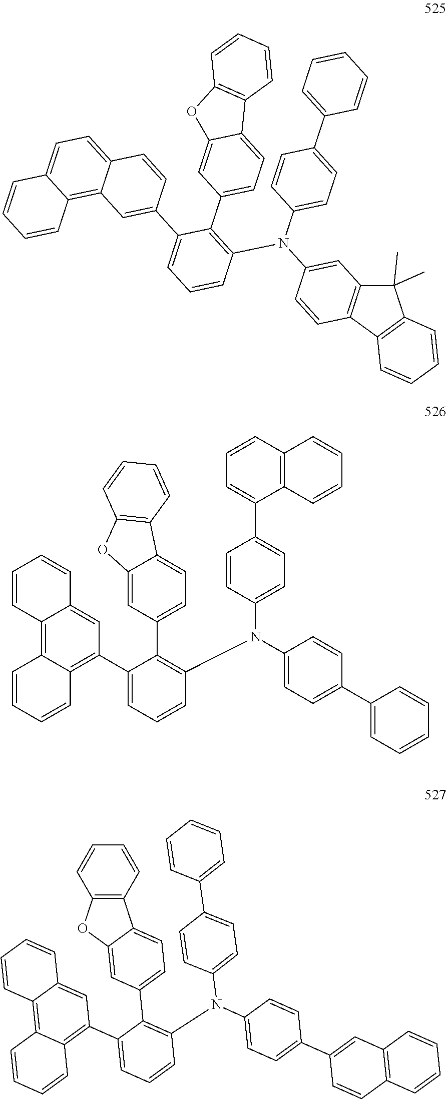 [SMILES: CC1(C)c2ccccc2-c2ccc(N(c3ccc(-c4ccccc4)cc3)c3cccc(-c4ccc5ccc6ccccc6c5c4)c3-c3ccc4c(c3)oc3ccccc34)cc21 ; c1ccc(-c2ccc(N(c3ccc(-c4cccc5ccccc45)cc3)c3cccc(-c4cc5ccccc5c5ccccc45)c3-c3ccc4c(c3)oc3ccccc34)cc2)cc1 ; c1ccc(-c2ccc(N(c3ccc(-c4ccc5ccccc5c4)cc3)c3cccc(-c4cc5ccccc5c5ccccc45)c3-c3ccc4c(c3)oc3ccccc34)cc2)cc1]

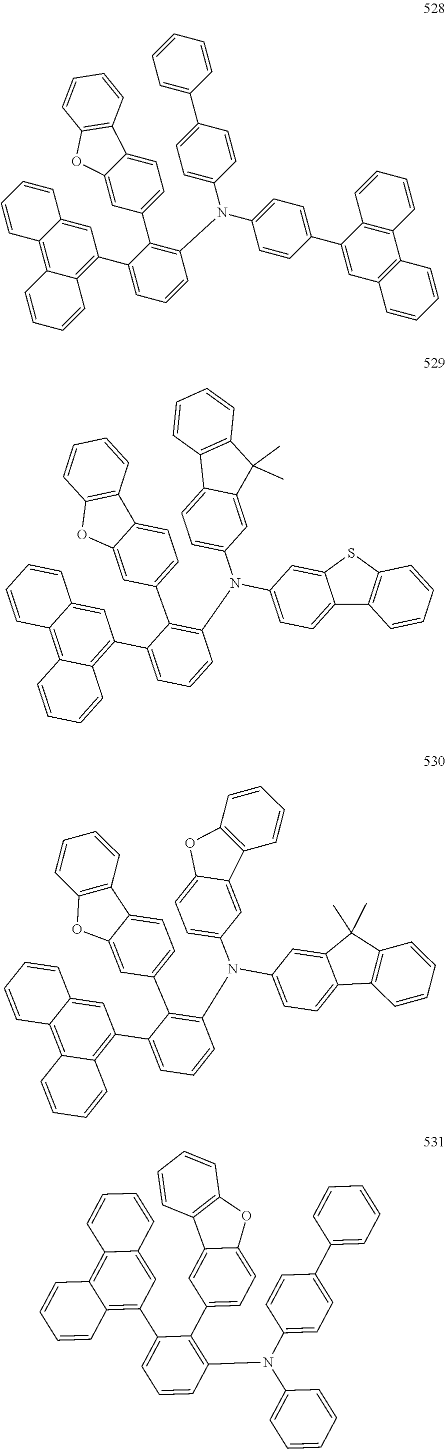 [SMILES: c1ccc(-c2ccc(N(c3ccc(-c4cc5ccccc5c5ccccc45)cc3)c3cccc(-c4cc5ccccc5c5ccccc45)c3-c3ccc4c(c3)oc3ccccc34)cc2)cc1 ; CC1(C)c2ccccc2-c2ccc(N(c3ccc4c(c3)sc3ccccc34)c3cccc(-c4cc5ccccc5c5ccccc45)c3-c3ccc4c(c3)oc3ccccc34)cc21 ; CC1(C)c2ccccc2-c2ccc(N(c3ccc4oc5ccccc5c4c3)c3cccc(-c4cc5ccccc5c5ccccc45)c3-c3ccc4c(c3)oc3ccccc34)cc21 ; c1ccc(-c2ccc(N(c3ccccc3)c3cccc(-c4cc5ccccc5c5ccccc45)c3-c3ccc4oc5ccccc5c4c3)cc2)cc1]

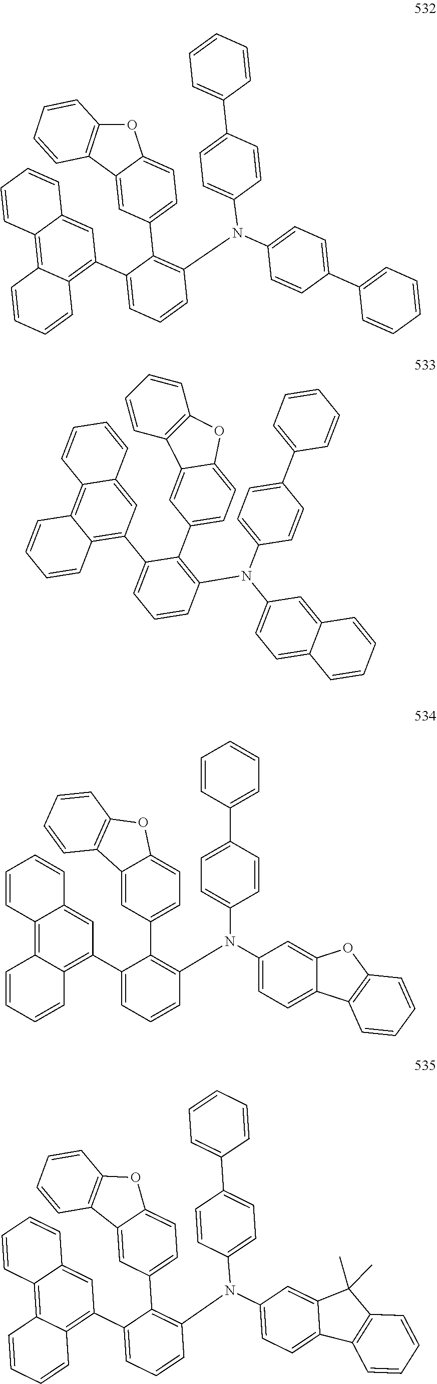 [SMILES: c1ccc(-c2ccc(N(c3ccc(-c4ccccc4)cc3)c3cccc(-c4cc5ccccc5c5ccccc45)c3-c3ccc4oc5ccccc5c4c3)cc2)cc1 ; c1ccc(-c2ccc(N(c3ccc4ccccc4c3)c3cccc(-c4cc5ccccc5c5ccccc45)c3-c3ccc4oc5ccccc5c4c3)cc2)cc1 ; c1ccc(-c2ccc(N(c3ccc4c(c3)oc3ccccc34)c3cccc(-c4cc5ccccc5c5ccccc45)c3-c3ccc4oc5ccccc5c4c3)cc2)cc1 ; CC1(C)c2ccccc2-c2ccc(N(c3ccc(-c4ccccc4)cc3)c3cccc(-c4cc5ccccc5c5ccccc45)c3-c3ccc4oc5ccccc5c4c3)cc21]

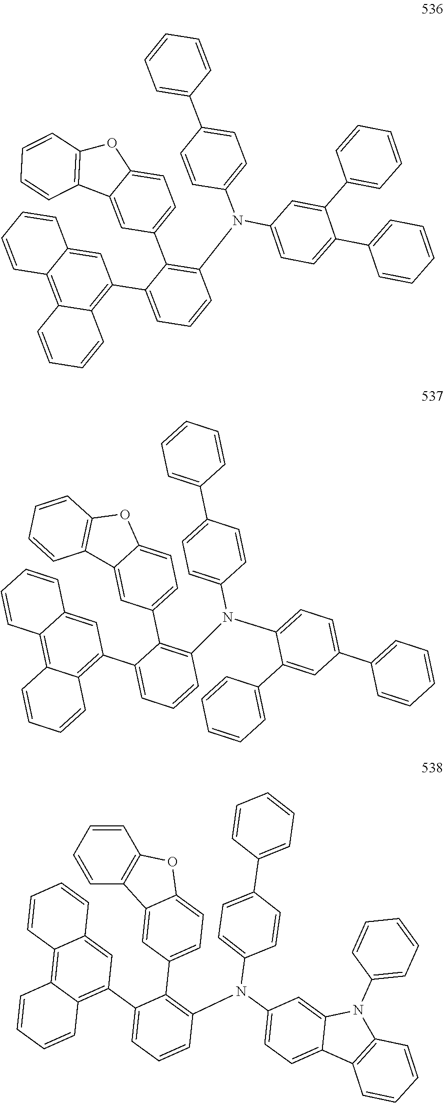 [SMILES: c1ccc(-c2ccc(N(c3ccc(-c4ccccc4)c(-c4ccccc4)c3)c3cccc(-c4cc5ccccc5c5ccccc45)c3-c3ccc4oc5ccccc5c4c3)cc2)cc1 ; c1ccc(-c2ccc(N(c3ccc(-c4ccccc4)cc3-c3ccccc3)c3cccc(-c4cc5ccccc5c5ccccc45)c3-c3ccc4oc5ccccc5c4c3)cc2)cc1 ; c1ccc(-c2ccc(N(c3ccc4c5ccccc5n(-c5ccccc5)c4c3)c3cccc(-c4cc5ccccc5c5ccccc45)c3-c3ccc4oc5ccccc5c4c3)cc2)cc1]

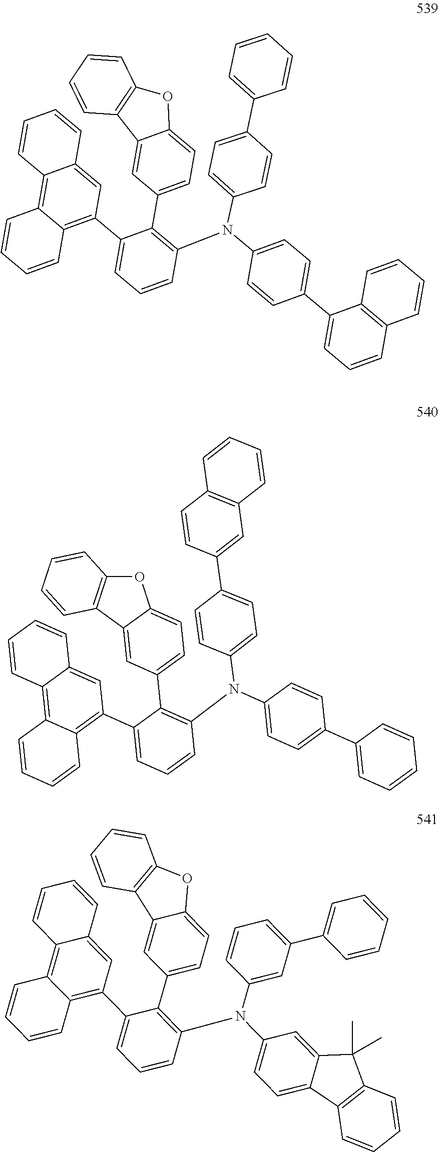 [SMILES: c1ccc(-c2ccc(N(c3ccc(-c4cccc5ccccc45)cc3)c3cccc(-c4cc5ccccc5c5ccccc45)c3-c3ccc4oc5ccccc5c4c3)cc2)cc1 ; c1ccc(-c2ccc(N(c3ccc(-c4ccc5ccccc5c4)cc3)c3cccc(-c4cc5ccccc5c5ccccc45)c3-c3ccc4oc5ccccc5c4c3)cc2)cc1 ; CC1(C)c2ccccc2-c2ccc(N(c3cccc(-c4ccccc4)c3)c3cccc(-c4cc5ccccc5c5ccccc45)c3-c3ccc4oc5ccccc5c4c3)cc21]

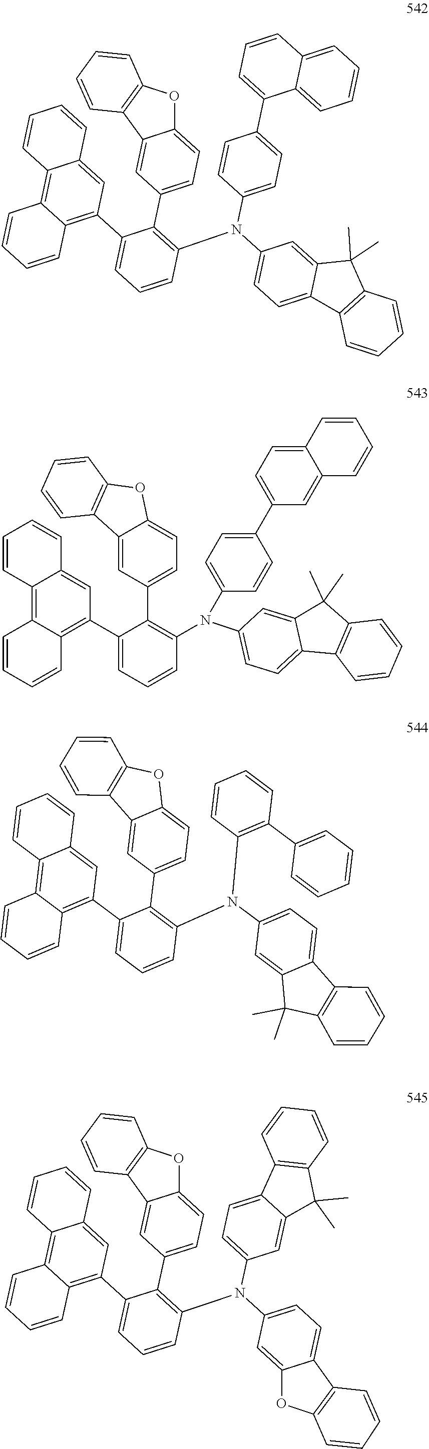 [SMILES: CC1(C)c2ccccc2-c2ccc(N(c3ccc(-c4cccc5ccccc45)cc3)c3cccc(-c4cc5ccccc5c5ccccc45)c3-c3ccc4oc5ccccc5c4c3)cc21 ; CC1(C)c2ccccc2-c2ccc(N(c3ccc(-c4ccc5ccccc5c4)cc3)c3cccc(-c4cc5ccccc5c5ccccc45)c3-c3ccc4oc5ccccc5c4c3)cc21 ; CC1(C)c2ccccc2-c2ccc(N(c3ccccc3-c3ccccc3)c3cccc(-c4cc5ccccc5c5ccccc45)c3-c3ccc4oc5ccccc5c4c3)cc21 ; CC1(C)c2ccccc2-c2ccc(N(c3ccc4c(c3)oc3ccccc34)c3cccc(-c4cc5ccccc5c5ccccc45)c3-c3ccc4oc5ccccc5c4c3)cc21]

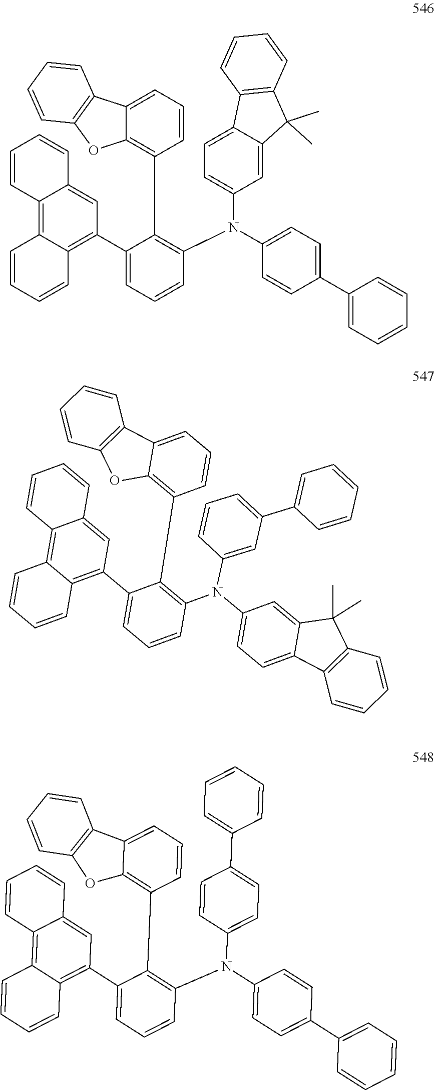 [SMILES: CC1(C)c2ccccc2-c2ccc(N(c3ccc(-c4ccccc4)cc3)c3cccc(-c4cc5ccccc5c5ccccc45)c3-c3cccc4c3oc3ccccc34)cc21 ; CC1(C)c2ccccc2-c2ccc(N(c3cccc(-c4ccccc4)c3)c3cccc(-c4cc5ccccc5c5ccccc45)c3-c3cccc4c3oc3ccccc34)cc21 ; c1ccc(-c2ccc(N(c3ccc(-c4ccccc4)cc3)c3cccc(-c4cc5ccccc5c5ccccc45)c3-c3cccc4c3oc3ccccc34)cc2)cc1]

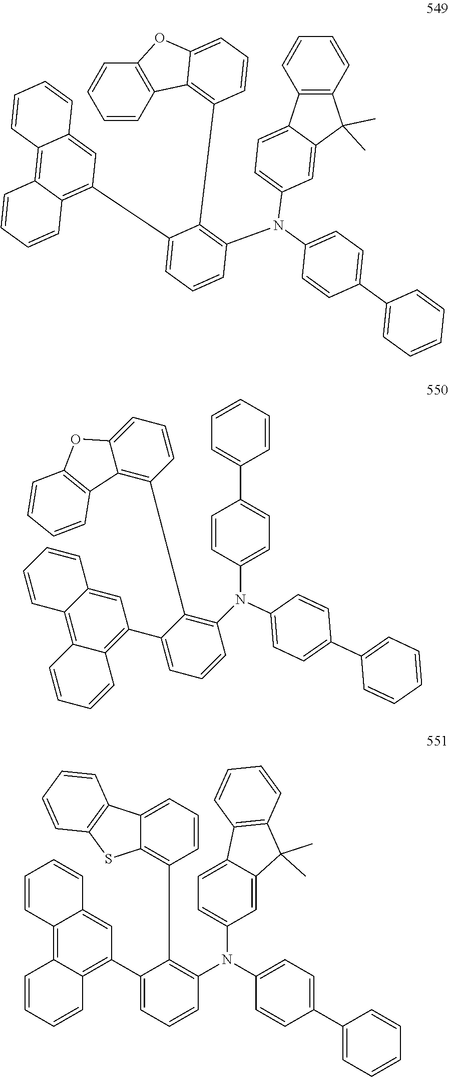 [SMILES: CC1(C)c2ccccc2-c2ccc(N(c3ccc(-c4ccccc4)cc3)c3cccc(-c4cc5ccccc5c5ccccc45)c3-c3cccc4oc5ccccc5c34)cc21 ; c1ccc(-c2ccc(N(c3ccc(-c4ccccc4)cc3)c3cccc(-c4cc5ccccc5c5ccccc45)c3-c3cccc4oc5ccccc5c34)cc2)cc1 ; CC1(C)c2ccccc2-c2ccc(N(c3ccc(-c4ccccc4)cc3)c3cccc(-c4cc5ccccc5c5ccccc45)c3-c3cccc4c3sc3ccccc34)cc21]

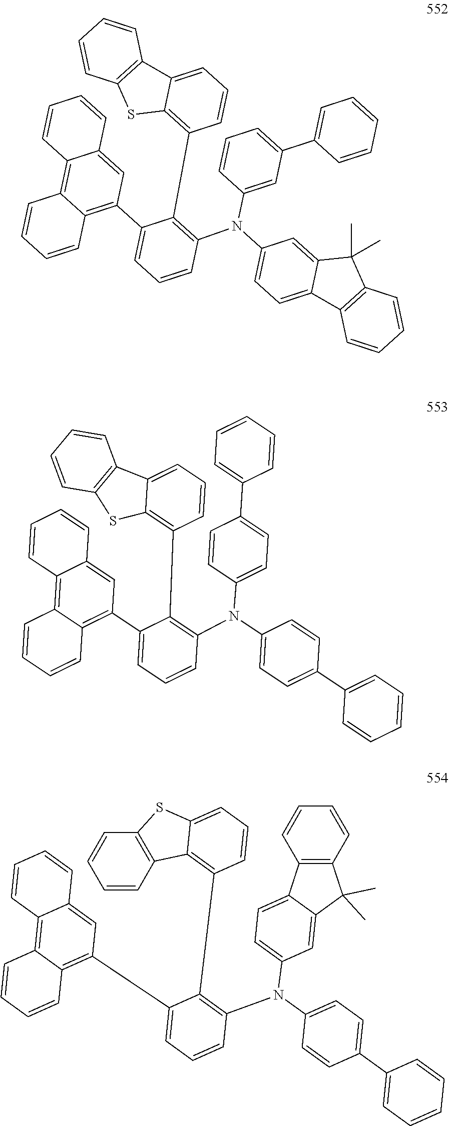 [SMILES: CC1(C)c2ccccc2-c2ccc(N(c3cccc(-c4ccccc4)c3)c3cccc(-c4cc5ccccc5c5ccccc45)c3-c3cccc4c3sc3ccccc34)cc21 ; c1ccc(-c2ccc(N(c3ccc(-c4ccccc4)cc3)c3cccc(-c4cc5ccccc5c5ccccc45)c3-c3cccc4c3sc3ccccc34)cc2)cc1 ; CC1(C)c2ccccc2-c2ccc(N(c3ccc(-c4ccccc4)cc3)c3cccc(-c4cc5ccccc5c5ccccc45)c3-c3cccc4sc5ccccc5c34)cc21]

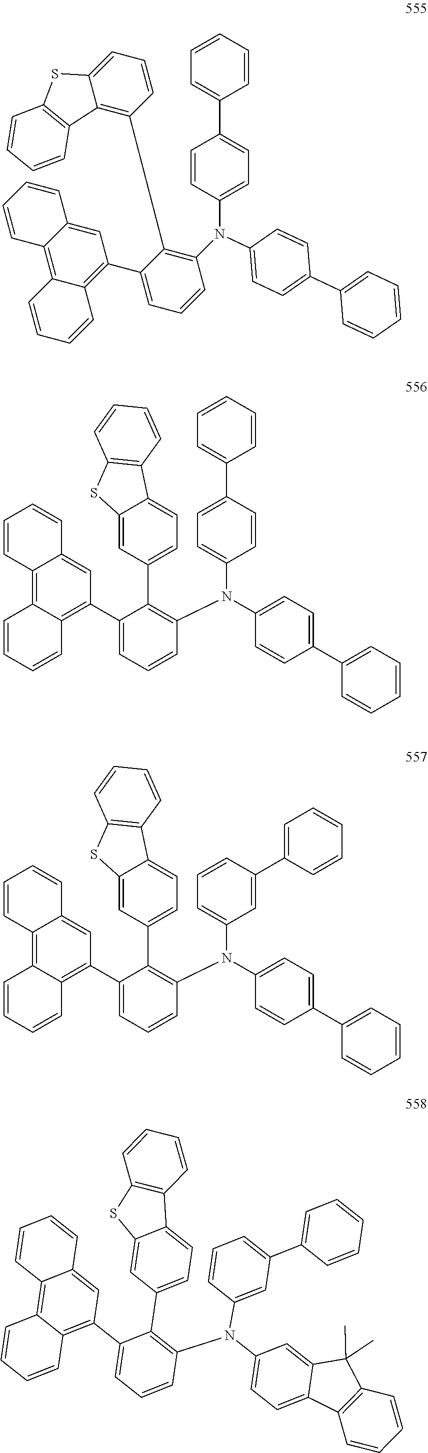 [SMILES: c1ccc(-c2ccc(N(c3ccc(-c4ccccc4)cc3)c3cccc(-c4cc5ccccc5c5ccccc45)c3-c3cccc4sc5ccccc5c34)cc2)cc1 ; c1ccc(-c2ccc(N(c3ccc(-c4ccccc4)cc3)c3cccc(-c4cc5ccccc5c5ccccc45)c3-c3ccc4c(c3)sc3ccccc34)cc2)cc1 ; c1ccc(-c2ccc(N(c3cccc(-c4ccccc4)c3)c3cccc(-c4cc5ccccc5c5ccccc45)c3-c3ccc4c(c3)sc3ccccc34)cc2)cc1 ; CC1(C)c2ccccc2-c2ccc(N(c3cccc(-c4ccccc4)c3)c3cccc(-c4cc5ccccc5c5ccccc45)c3-c3ccc4c(c3)sc3ccccc34)cc21]

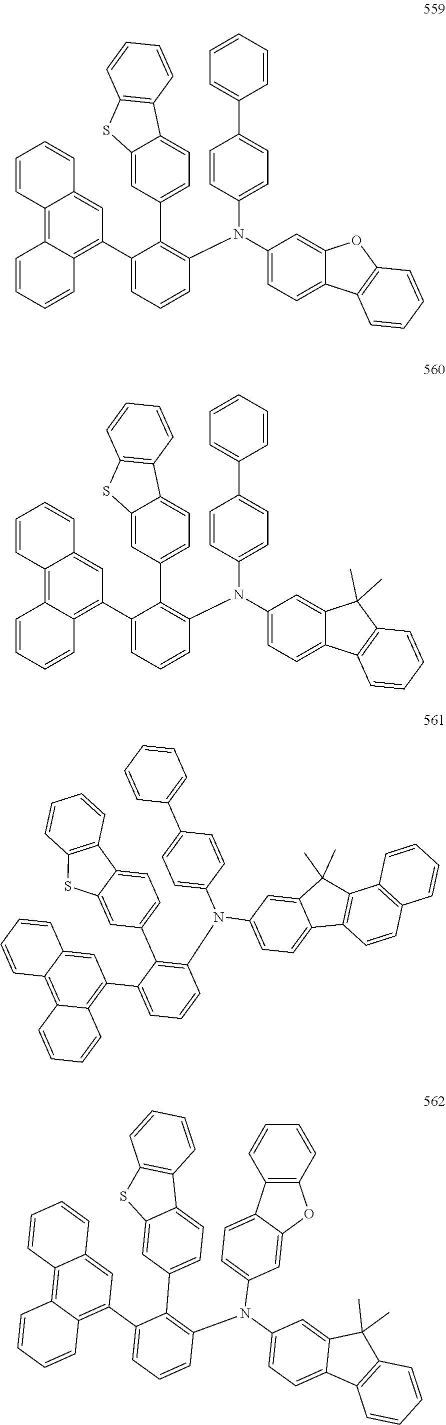 [SMILES: c1ccc(-c2ccc(N(c3ccc4c(c3)oc3ccccc34)c3cccc(-c4cc5ccccc5c5ccccc45)c3-c3ccc4c(c3)sc3ccccc34)cc2)cc1 ; CC1(C)c2ccccc2-c2ccc(N(c3ccc(-c4ccccc4)cc3)c3cccc(-c4cc5ccccc5c5ccccc45)c3-c3ccc4c(c3)sc3ccccc34)cc21 ; CC1(C)c2cc(N(c3ccc(-c4ccccc4)cc3)c3cccc(-c4cc5ccccc5c5ccccc45)c3-c3ccc4c(c3)sc3ccccc34)ccc2-c2ccc3ccccc3c21 ; CC1(C)c2ccccc2-c2ccc(N(c3ccc4c(c3)oc3ccccc34)c3cccc(-c4cc5ccccc5c5ccccc45)c3-c3ccc4c(c3)sc3ccccc34)cc21]

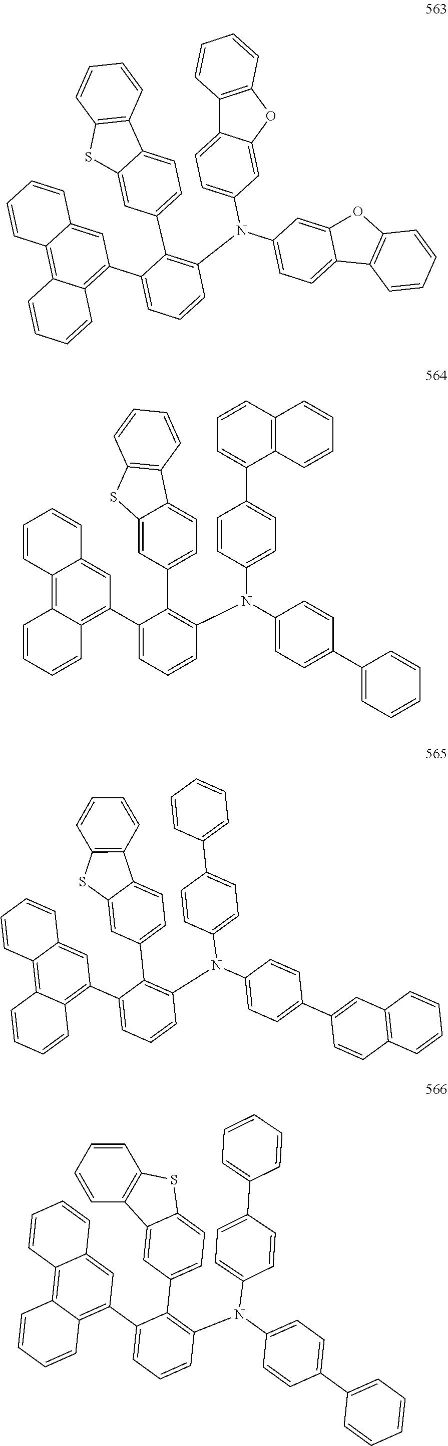 [SMILES: c1cc(-c2cc3ccccc3c3ccccc23)c(-c2ccc3c(c2)sc2ccccc23)c(N(c2ccc3c(c2)oc2ccccc23)c2ccc3c(c2)oc2ccccc23)c1 ; c1ccc(-c2ccc(N(c3ccc(-c4cccc5ccccc45)cc3)c3cccc(-c4cc5ccccc5c5ccccc45)c3-c3ccc4c(c3)sc3ccccc34)cc2)cc1 ; c1ccc(-c2ccc(N(c3ccc(-c4ccc5ccccc5c4)cc3)c3cccc(-c4cc5ccccc5c5ccccc45)c3-c3ccc4c(c3)sc3ccccc34)cc2)cc1 ; c1ccc(-c2ccc(N(c3ccc(-c4ccccc4)cc3)c3cccc(-c4cc5ccccc5c5ccccc45)c3-c3ccc4sc5ccccc5c4c3)cc2)cc1]

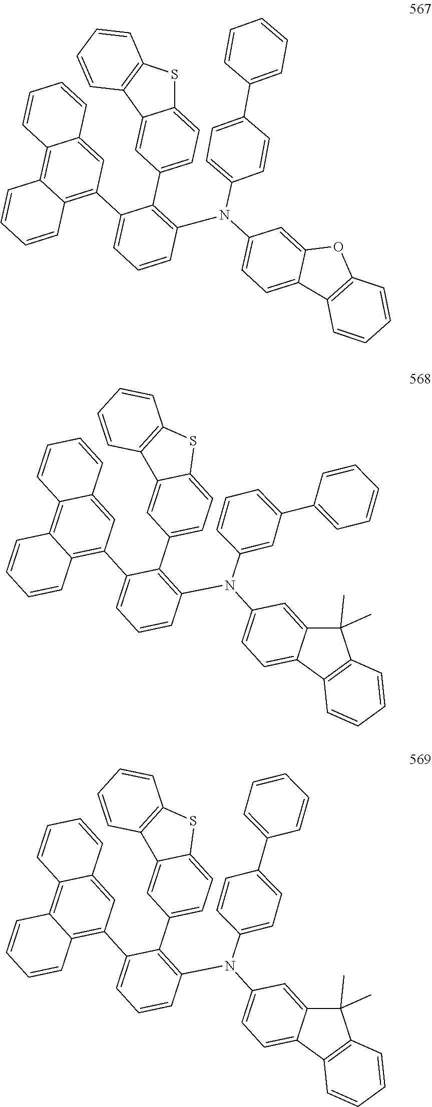 [SMILES: c1ccc(-c2ccc(N(c3ccc4c(c3)oc3ccccc34)c3cccc(-c4cc5ccccc5c5ccccc45)c3-c3ccc4sc5ccccc5c4c3)cc2)cc1 ; CC1(C)c2ccccc2-c2ccc(N(c3cccc(-c4ccccc4)c3)c3cccc(-c4cc5ccccc5c5ccccc45)c3-c3ccc4sc5ccccc5c4c3)cc21 ; CC1(C)c2ccccc2-c2ccc(N(c3ccc(-c4ccccc4)cc3)c3cccc(-c4cc5ccccc5c5ccccc45)c3-c3ccc4sc5ccccc5c4c3)cc21]

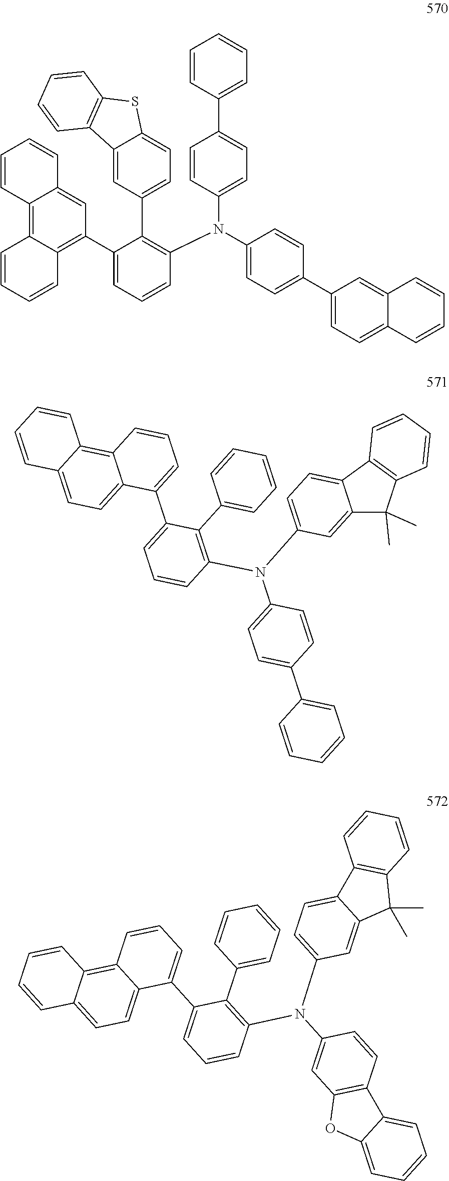 [SMILES: c1ccc(-c2ccc(N(c3ccc(-c4ccc5ccccc5c4)cc3)c3cccc(-c4cc5ccccc5c5ccccc45)c3-c3ccc4sc5ccccc5c4c3)cc2)cc1 ; CC1(C)c2ccccc2-c2ccc(N(c3ccc(-c4ccccc4)cc3)c3cccc(-c4cccc5c4ccc4ccccc45)c3-c3ccccc3)cc21 ; CC1(C)c2ccccc2-c2ccc(N(c3ccc4c(c3)oc3ccccc34)c3cccc(-c4cccc5c4ccc4ccccc45)c3-c3ccccc3)cc21]

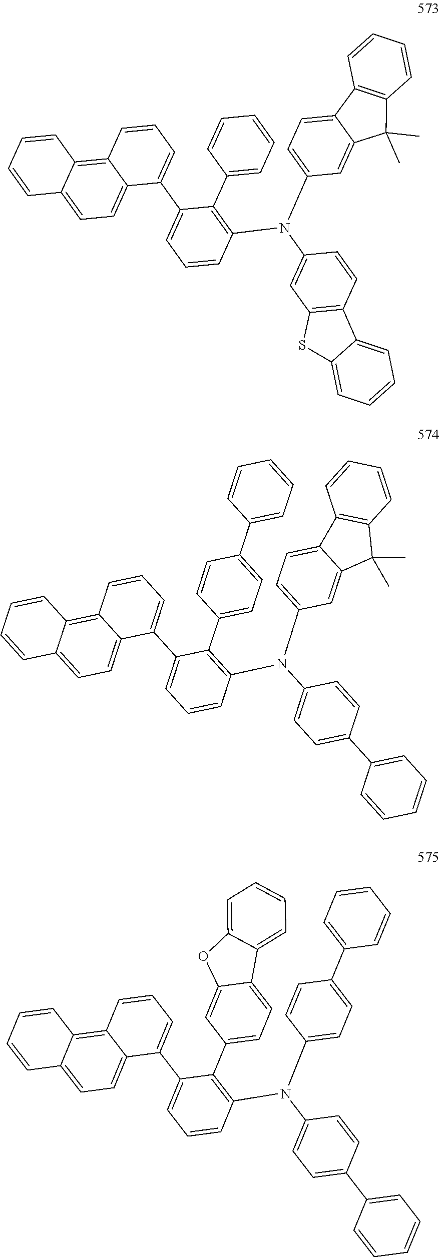 [SMILES: CC1(C)c2ccccc2-c2ccc(N(c3ccc4c(c3)sc3ccccc34)c3cccc(-c4cccc5c4ccc4ccccc45)c3-c3ccccc3)cc21 ; CC1(C)c2ccccc2-c2ccc(N(c3ccc(-c4ccccc4)cc3)c3cccc(-c4cccc5c4ccc4ccccc45)c3-c3ccc(-c4ccccc4)cc3)cc21 ; c1ccc(-c2ccc(N(c3ccc(-c4ccccc4)cc3)c3cccc(-c4cccc5c4ccc4ccccc45)c3-c3ccc4c(c3)oc3ccccc34)cc2)cc1]

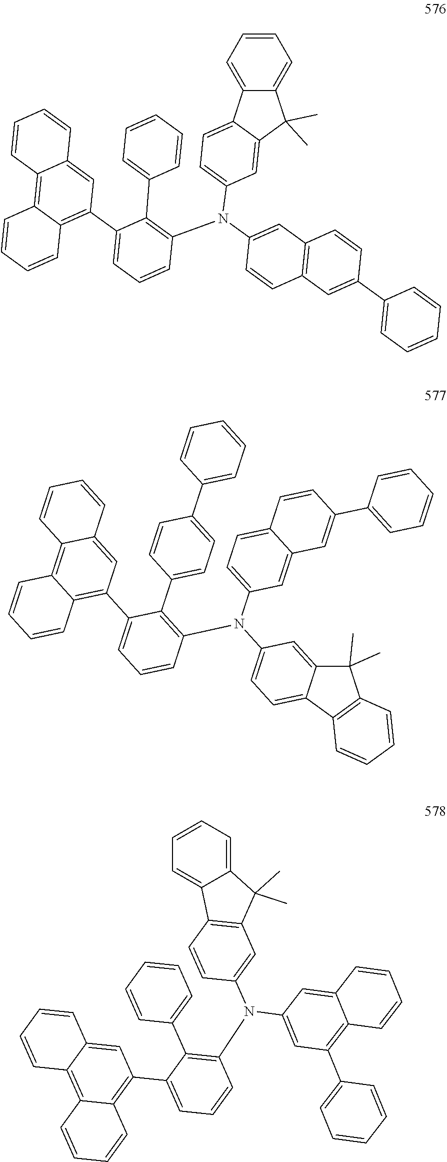 [SMILES: CC1(C)c2ccccc2-c2ccc(N(c3ccc4cc(-c5ccccc5)ccc4c3)c3cccc(-c4cc5ccccc5c5ccccc45)c3-c3ccccc3)cc21 ; CC1(C)c2ccccc2-c2ccc(N(c3ccc4ccc(-c5ccccc5)cc4c3)c3cccc(-c4cc5ccccc5c5ccccc45)c3-c3ccc(-c4ccccc4)cc3)cc21 ; CC1(C)c2ccccc2-c2ccc(N(c3cc(-c4ccccc4)c4ccccc4c3)c3cccc(-c4cc5ccccc5c5ccccc45)c3-c3ccccc3)cc21]

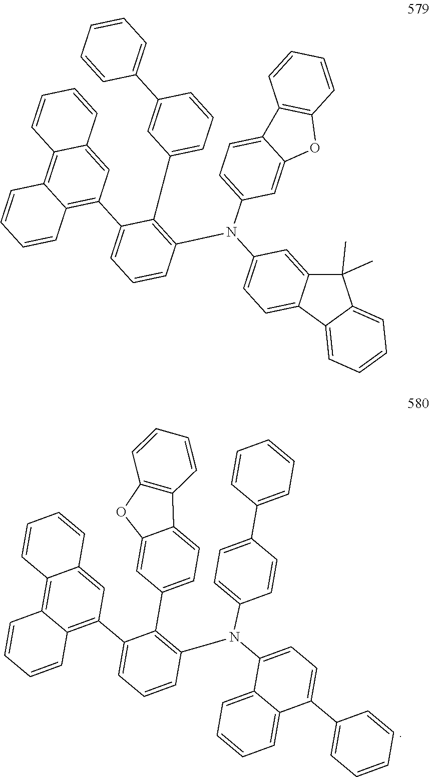 [SMILES: CC1(C)c2ccccc2-c2ccc(N(c3ccc4c(c3)oc3ccccc34)c3cccc(-c4cc5ccccc5c5ccccc45)c3-c3cccc(-c4ccccc4)c3)cc21 ; c1ccc(-c2ccc(N(c3cccc(-c4cc5ccccc5c5ccccc45)c3-c3ccc4c(c3)oc3ccccc34)c3ccc(-c4ccccc4)c4ccccc34)cc2)cc1]

14. An electronic element, comprising an anode and a cathode disposed opposite to each other, and a functional layer disposed between the anode and the cathode, wherein the functional layer comprises the organic compound of claim 1;
optionally, the electronic element is an organic electroluminescent device or a photoelectric conversion device.

15. The electronic element according to claim 14, wherein the functional layer comprises a second hole transport layer, and the second hole transport layer comprising the organic compound.

16. An electronic apparatus, comprising the electronic element according to claim 14.
